(19) United States
(12) Patent Application Publication
(10) Pub. No.: US 2023/0295115 A1
(43) Pub. Date: Sep. 21, 2023

(54) SUBSTITUTED ISOQUINOLINYLMETHYL AMIDES, ANALOGUES THEREOF, AND METHODS USING SAME

(71) Applicant: Arbutus Biopharma Corporation, Burnaby (CA)

(72) Inventors: Andrew G. Cole, Cranbury, NJ (US); Bruce D. Dorsey, Ambler, PA (US); Benjamin J. Dugan, Glen Mills, PA (US); Yi Fan, Doylestown, PA (US); Steven G. Kultgen, Warminster, PA (US); Eugen F. Mesaros, Wallingford, PA (US); Michael J. Sofia, Doylestown, PA (US)

(21) Appl. No.: 18/008,873

(22) PCT Filed: Jun. 7, 2021
(86) PCT No.: PCT/IB21/00382

§ 371 Date: Dec. 7, 2022

Related U.S. Application Data
(60) Provisional application No. 63/036,099, filed on Jun. 8, 2020.

(51) Int. Cl. C07D 401/12; A61K 45/06; C07K 16/28; C07D 405/12; C07D 471/04; C07D 217/24; C07D 495/04

(52) U.S. Cl. CPC C07D 401/12; A61K 45/06; C07K 16/2827; C07D 405/12; C07D 471/04; C07D 217/24; C07D 495/04

(57) ABSTRACT

The present disclosure includes substituted isoquinolinylmethyl amides, or analogues thereof, and compositions comprising the same, that can be used to treat, ameliorate, and/or prevent hepatitis B virus (HBV) and/or hepatitis D virus (HDV) infections in a patient.

### CROSS-REFERENCE TO RELATED APPLICATIONS

This application claims priority under 35 U.S.C. § 119(e) to U.S. Provisional Patent Application No. 63/036,099, filed Jun. 8, 2020, which is incorporated herein by reference in its entirety.

### BACKGROUND

Hepatitis B is one of the world's most prevalent diseases, being listed by National Institute of Allergy and Infectious Diseases (NIAID) as a High Priority Area of Interest. Although most individuals resolve the infection following acute symptoms, approximately 30% of cases become chronic. 350-400 million people worldwide are estimated to have chronic hepatitis B, leading to 0.5-1 million deaths per year, due largely to the development of hepatocellular carcinoma, cirrhosis and/or other complications.

A limited number of drugs are currently approved for the management of chronic hepatitis B, including two formulations of alpha-interferon (standard and pegylated) and five nucleoside/nucleotide analogues (lamivudine, adefovir, entecavir, telbivudine, and tenofovir) that inhibit hepatitis B virus (HBV) DNA polymerase. At present, the first-line treatment choices are entecavir, tenofovir and/or peg-interferon alfa-2a. However, peg-interferon alfa-2a achieves desirable serological milestones in only one third of treated patients, and is frequently associated with severe side effects. Entecavir and tenofovir are potent HBV inhibitors, but require long-term or possibly lifetime administration to continuously suppress HBV replication, and may eventually fail due to emergence of drug-resistant viruses. There is thus a pressing need for the introduction of novel, safe, and effective therapies for chronic hepatitis B.

HBV is a noncytopathic, liver tropic DNA virus belonging to Hepadnaviridae family. Pregenomic (pg) RNA is the template for reverse transcriptional replication of HBV DNA. The encapsidation of pg RNA, together with viral DNA polymerase, into a nucleocapsid is essential for the subsequent viral DNA synthesis. Inhibition of pg RNA encapsidation may block HBV replication and provide a new therapeutic approach to HBV treatment. A capsid inhibitor acts by inhibiting the expression and/or function of a capsid protein either directly or indirectly: for example, it may inhibit capsid assembly, induce formation of non-capsid polymers, promote excess capsid assembly or misdirected capsid assembly, affect capsid stabilization, and/or inhibit RNA encapsidation. A capsid inhibitor may also act by inhibiting capsid function in one or more downstream events within the replication process, such as, but not limited to, viral DNA synthesis, transport of relaxed circular DNA (rcDNA) into the nucleus, covalently closed circular DNA (cccDNA) formation, virus maturation, budding and/or release.

Clinically, inhibition of pg RNA encapsidation, or more generally inhibition of nucleocapsid assembly, may offer certain therapeutic advantages. In one aspect, inhibition of pg RNA encapsidation may complement the current medications by providing an option for a subpopulation of patients that do not tolerate or benefit from the current medications. In another aspect, based on their distinct antiviral mechanism, inhibition of pg RNA encapsidation may be effective against HBV variants resistant to the currently available DNA polymerase inhibitors. In yet another aspect, combination therapy of the pg RNA encapsidation inhibitors with DNA polymerase inhibitors may synergistically suppress HBV replication and prevent drug resistance emergence, thus offering a more effective treatment for chronic hepatitis B infection.

Hepatitis D virus (HDV) is a small circular enveloped RNA virus that can propagate only in the presence of HBV. In particular, HDV requires the HBV surface antigen protein to propagate itself. Infection with both HBV and HDV results in more severe complications compared to infection with HBV alone. These complications include a greater likelihood of experiencing liver failure in acute infections and a rapid progression to liver cirrhosis, with an increased chance of developing liver cancer in chronic infections. In combination with hepatitis B, hepatitis D has the highest mortality rate of all the hepatitis infections. The routes of transmission of HDV are similar to those for HBV. Infection is largely restricted to persons at high risk of HBV infection, particularly injecting drug users and persons receiving clotting factor concentrates.

Currently, there is no effective antiviral therapy available for the treatment of acute or chronic type D hepatitis. Interferon-alfa given weekly for 12 to 18 months is the only licensed treatment for hepatitis D. Response to this therapy is limited, as only about one-quarter of patients is serum HDV RNA undetectable 6 months post therapy.

Clinically, inhibition of pg RNA encapsidation, or more generally inhibition of nucleocapsid assembly, may offer certain therapeutic advantages for treatment of hepatitis B and/or hepatitis D. In one aspect, inhibition of pg RNA encapsidation may complement the current medications by providing an option for a subpopulation of patients that do not tolerate or benefit from the current medications. In another aspect, based on their distinct antiviral mechanism, inhibition of pg RNA encapsidation may be effective against HBV and/or HDV variants resistant to the currently available DNA polymerase inhibitors. In yet another aspect, combination therapy of the pg RNA encapsidation inhibitors with DNA polymerase inhibitors may synergistically suppress HBV and/or HDV replication and prevent drug resistance emergence, thus offering a more effective treatment for chronic hepatitis B and/or hepatis D infection.

There is thus a need in the art for the identification of novel compounds that can be used to treat, ameliorate, and/or prevent HBV and/or HDV infection in a subject. In certain embodiments, the novel compounds inhibit HBV and/or HDV nucleocapsid assembly. In other embodiments, the novel compounds can be used in patients that are HBV and/or HBV-HDV infected, patients who are at risk of becoming HBV and/or HBV-HDV infected, and/or patients that are infected with drug-resistant HBV and/or HDV. The present invention addresses this need.

### BRIEF SUMMARY

The present disclosure provides certain compounds of formula (I), or a salt, solvate, prodrug, stereoisomer, tautomer, or isotopically labeled derivative thereof, or any mixtures thereof, wherein the substituents in (I) are defined elsewhere herein:

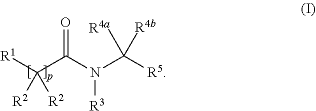

The present disclosure further provides pharmaceutical compositions comprising at least one compound of the present disclosure. In certain embodiments, the pharmaceutical compositions further comprise at least one pharmaceutically acceptable carrier. In other embodiments, the pharmaceutical compositions further comprise at least one additional agent useful for treating, ameliorating, and/or preventing hepatitis infection. In yet other embodiments, the hepatitis virus is hepatitis B virus (HBV). In yet other embodiments, the hepatitis virus is hepatitis D virus (HDV).

The present disclosure further provides methods of treating, ameliorating, and/or preventing hepatitis virus infection in a subject. In certain embodiments, the method comprises administering to the subject a therapeutically effective amount of at least one compound of the present disclosure and/or at least one pharmaceutical composition of the present disclosure. In other embodiments, the subject is infected with HBV. In yet other embodiments, the subject is infected with HBV and HDV. In yet other embodiments, the subject is further administered at least one additional agent useful for treating, ameliorating, and/or preventing the hepatitis virus infection. In yet other embodiments, the subject is in need of the treatment, amelioration, and/or prevention.

### DETAILED DESCRIPTION

The disclosure relates, in certain aspects, to the discovery of certain substituted ureas and amides that are useful to treat, ameliorate, and/or prevent hepatitis B virus (HBV) and/or hepatitis D virus (HDV) infection and related conditions in a subject. In certain embodiments, the compounds of the disclosure are viral capsid inhibitors.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, separation science, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

As used herein, the term “about” will be understood by persons of ordinary skill in the art and will vary to some extent on the context in which it is used. As used herein, “about” when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

As used herein, the term “alkenyl,” employed alone or in combination with other terms, means, unless otherwise stated, a stable monounsaturated or diunsaturated straight chain or branched chain hydrocarbon group having the stated number of carbon atoms. Examples include vinyl, propenyl (or allyl), crotyl, isopentenyl, butadienyl, 1,3-pentadienyl, 1,4-pentadienyl, and the higher homologs and isomers. A functional group representing an alkene is exemplified by —CH₂—CH═CH₂.

As used herein, the term “alkoxy” employed alone or in combination with other terms means, unless otherwise stated, an alkyl group having the designated number of carbon atoms, as defined elsewhere herein, connected to the rest of the molecule via an oxygen atom, such as, for example, methoxy, ethoxy, 1-propoxy, 2-propoxy (or isopropoxy) and the higher homologs and isomers. A specific example is (C₁-C₃)alkoxy, such as, but not limited to, ethoxy and methoxy.

As used herein, the term “alkyl” by itself or as part of another substituent means, unless otherwise stated, a straight or branched chain hydrocarbon having the number of carbon atoms designated (i.e., C₁-C₁₀ means one to ten carbon atoms) and includes straight, branched chain, or cyclic substituent groups. Examples include methyl, ethyl, propyl, isopropyl, butyl, isobutyl, tert-butyl, pentyl, neopentyl, hexyl, and cyclopropylmethyl. A specific embodiment is (C₁-C₆)alkyl, such as, but not limited to, ethyl, methyl, isopropyl, isobutyl, n-pentyl, n-hexyl, and cyclopropylmethyl.

As used herein, the term “alkynyl” employed alone or in combination with other terms means, unless otherwise stated, a stable straight chain or branched chain hydrocarbon group with a triple carbon-carbon bond, having the stated number of carbon atoms. Non-limiting examples include ethynyl and propynyl, and the higher homologs and isomers. The term “propargylic” refers to a group exemplified by —CH₂—C≡CH. The term “homopropargylic” refers to a group exemplified by —CH₂CH₂—C≡CH.

As used herein, the term “aromatic” refers to a carbocycle or heterocycle with one or more polyunsaturated rings and having aromatic character, i.e., having (4n+2) delocalized π (pi) electrons, where ‘n’ is an integer.

As used herein, the term “aryl” employed alone or in combination with other terms means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl and naphthyl. Aryl groups also include, for example, phenyl or naphthyl rings fused with one or more saturated or partially saturated carbon rings (e.g., bicyclo[4.2.0]octa-1,3,5-trienyl, or indanyl), which can be substituted at one or more carbon atoms of the aromatic and/or saturated or partially saturated rings.

As used herein, the term “aryl-(C₁-C₆)alkyl” refers to a functional group wherein a one-to-six carbon alkylene chain is attached to an aryl group, e.g., —CH₂CH₂-phenyl or —CH₂-phenyl (or benzyl). Specific examples are aryl-CH₂— and aryl-CH(CH₃)—. The term “substituted aryl-(C₁-C₆)alkyl” refers to an aryl-(C₁-C₆)alkyl functional group in which the aryl group is substituted. A specific example is substituted aryl(CH₂)—. Similarly, the term “heteroaryl-(C₁-C₆)alkyl” refers to a functional group wherein a one-to-three carbon alkylene chain is attached to a heteroaryl group, e.g., —CH₂CH₂-pyridyl. A specific example is heteroaryl-(CH₂)—. The term “substituted heteroaryl-(C₁-C₆)alkyl” refers to a heteroaryl-(C₁-C₆)alkyl functional group in which the heteroaryl group is substituted. A specific example is substituted heteroaryl-(CH₂)—.

In one aspect, the terms “co-administered” and “co-administration” as relating to a subject refer to administering to the subject a compound and/or composition of the disclosure along with a compound and/or composition that may also treat, ameliorate, and/or prevent a disease or disorder contemplated herein. In certain embodiments, the co-administered compounds and/or compositions are administered separately, or in any kind of combination as part of a single therapeutic approach. The co-administered compound and/or composition may be formulated in any kind of combinations as mixtures of solids and liquids under a variety of solid, gel, and liquid formulations, and as a solution.

As used herein, the term “cycloalkyl” by itself or as part of another substituent refers to, unless otherwise stated, a cyclic chain hydrocarbon having the number of carbon atoms designated (i.e., C₃-C₆ refers to a cyclic group comprising a ring group consisting of three to six carbon atoms) and includes straight, branched chain or cyclic substituent groups. Examples of (C₃-C₆)cycloalkyl groups are cyclopropyl, cyclobutyl, cyclopentyl and cyclohexyl. Cycloalkyl rings can be optionally substituted. Non-limiting examples of cycloalkyl groups include: cyclopropyl, 2-methyl-cyclopropyl, cyclopropenyl, cyclobutyl, 2,3-dihydroxycyclobutyl, cyclobutenyl, cyclopentyl, cyclopentenyl, cyclopentadienyl, cyclohexyl, cyclohexenyl, cycloheptyl, cyclooctanyl, decalinyl, 2,5-dimethylcyclopentyl, 3,5-dichlorocyclohexyl, 4-hydroxycyclohexyl, 3,3,5-trimethylcyclohex-1-yl, octahydropentalenyl, octahydro-1H-indenyl, 3a,4,5,6,7,7a-hexahydro-3H-inden-4-yl, decahydroazulenyl; bicyclo[6.2.0]decanyl, decahydronaphthalenyl, and dodecahydro-1H-fluorenyl. The term “cycloalkyl” also includes bicyclic hydrocarbon rings, non-limiting examples of which include, bicyclo[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

As used herein, a “disease” is a state of health of a subject wherein the subject cannot maintain homeostasis, and wherein if the disease is not ameliorated then the subject's health continues to deteriorate.

As used herein, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

As used herein, the term “halide” refers to a halogen atom bearing a negative charge. The halide anions are fluoride (F⁻), chloride (Cl⁻), bromide (Br⁻), and iodide (I⁻).

As used herein, the term “halo” or “halogen” alone or as part of another substituent refers to, unless otherwise stated, a fluorine, chlorine, bromine, or iodine atom.

As used herein, the term “heteroalkenyl” by itself or in combination with another term refers to, unless otherwise stated, a stable straight or branched chain monounsaturated or diunsaturated hydrocarbon group consisting of the stated number of carbon atoms and one or two heteroatoms selected from the group consisting of O, N, and S, and wherein the nitrogen and sulfur atoms may optionally be oxidized and the nitrogen heteroatom may optionally be quaternized. Up to two heteroatoms may be placed consecutively. Examples include —CH═CH—O—CH₃, —CH═CH—CH₂—OH, —CH₂—CH═N—OCH₃, —CH═CH—N(CH₃)—CH₃, and —CH₂—CH═CH—CH₂—SH.

As used herein, the term “heteroalkyl” by itself or in combination with another term refers to, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —OCH₂CH₂CH₃, —CH₂CH₂CH₂OH, —CH₂CH₂NHCH₃, —CH₂SCH₂CH₃, and —CH₂CH₂S(═O)CH₃. Up to two heteroatoms may be consecutive, such as, for example, —CH₂NH—OCH₃, or —CH₂CH₂SSCH₃.

As used herein, the term “heteroaryl” or “heteroaromatic” refers to a heterocycle having aromatic character. A polycyclic heteroaryl may include one or more rings that are partially saturated. Examples include tetrahydroquinoline and 2,3-dihydrobenzofuryl.

As used herein, the term “heterocycle” or “heterocyclyl” or “heterocyclic” by itself or as part of another substituent refers to, unless otherwise stated, an unsubstituted or substituted, stable, mono- or multi-cyclic heterocyclic ring system that comprises carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In certain embodiments, the heterocycle is a heteroaryl.

Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin, and hexamethyleneoxide.

Examples of heteroaryl groups include pyridyl, pyrazinyl, pyrimidinyl (such as, but not limited to, 2- and 4-pyrimidinyl), pyridazinyl, thienyl, furyl, pyrrolyl, imidazolyl, thiazolyl, oxazolyl, pyrazolyl, isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl, and 1,3,4-oxadiazolyl.

Examples of polycyclic heterocycles include indolyl (such as, but not limited to, 3-, 4-, 5-, 6- and 7-indolyl), indolinyl, quinolyl, tetrahydroquinolyl, isoquinolyl (such as, but not limited to, 1- and 5-isoquinolyl), 1,2,3,4-tetrahydroisoquinolyl, cinnolinyl, quinoxalinyl (such as, but not limited to, 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (such as, but not limited to, 3-, 4-, 5-, 6- and 7-benzofuryl), 2,3-dihydrobenzofuryl, 1,2-benzisoxazolyl, benzothienyl (such as, but not limited to, 3-, 4-, 5-, 6-, and 7-benzothienyl), benzoxazolyl, benzothiazolyl (such as, but not limited to, 2-benzothiazolyl and 5-benzothiazolyl), purinyl, benzimidazolyl, benztriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

As used herein, the term “pharmaceutical composition” or “composition” refers to a mixture of at least one compound useful within the disclosure with a pharmaceutically acceptable carrier. The pharmaceutical composition facilitates administration of the compound to a subject.

As used herein, the term “pharmaceutically acceptable” refers to a material, such as a carrier or diluent, which does not abrogate the biological activity or properties of the compound useful within the disclosure, and is relatively non-toxic, i.e., the material may be administered to a subject without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the disclosure, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the disclosure, and are physiologically acceptable to the subject. Supplementary active compounds may also be incorporated into the compositions. The “pharmaceutically acceptable carrier” may further include a pharmaceutically acceptable salt of the compound useful within the disclosure. Other additional ingredients that may be included in the pharmaceutical compositions used in the practice of the disclosure are known in the art and described, for example in Remington's Pharmaceutical Sciences (Genaro, Ed., Mack Publishing Co., 1985, Easton, PA), which is incorporated herein by reference.

As used herein, the language “pharmaceutically acceptable salt” refers to a salt of the administered compound prepared from pharmaceutically acceptable non-toxic acids and/or bases, including inorganic acids, inorganic bases, organic acids, inorganic bases, solvates (including hydrates) and clathrates thereof.

As used herein, a “pharmaceutically effective amount,” “therapeutically effective amount,” or “effective amount” of a compound is that amount of compound that is sufficient to provide a beneficial effect to the subject to which the compound is administered.

The term “prevent,” “preventing,” or “prevention” as used herein means avoiding or delaying the onset of symptoms associated with a disease or condition in a subject that has not developed such symptoms at the time the administering of an agent or compound commences. Disease, condition and disorder are used interchangeably herein.

By the term “specifically bind” or “specifically binds” as used herein is meant that a first molecule preferentially binds to a second molecule (e.g., a particular receptor or enzyme), but does not necessarily bind only to that second molecule.

As used herein, the terms “subject” and “individual” and “patient” can be used interchangeably and may refer to a human or non-human mammal or a bird. Non-human mammals include, for example, livestock and pets, such as ovine, bovine, porcine, canine, feline and murine mammals. In certain embodiments, the subject is human.

As used herein, the term “substituted” refers to that an atom or group of atoms has replaced hydrogen as the substituent attached to another group.

As used herein, the term “substituted alkyl,” “substituted cycloalkyl,” “substituted alkenyl,” or “substituted alkynyl” refers to alkyl, cycloalkyl, alkenyl, or alkynyl, as defined elsewhere herein, substituted by one, two or three substituents independently selected from the group consisting of halogen, —OH, alkoxy, tetrahydro-2-H-pyranyl, —NH₂, —NH(C₁-C₆ alkyl), —N(C₁-C₆ alkyl)₂, 1-methyl-imidazol-2-yl, pyridin-2-yl, pyridin-3-yl, pyridin-4-yl, —C(═O)OH, —C(═O)O(C₁-C₆)alkyl, trifluoromethyl, —C≡N, —C(═O)NH₂, —C(═O)NH(C₁-C₆)alkyl, —C(═O)N((C₁-C₆)alkyl)₂, —SO₂NH₂, —SO₂NH(C₁-C₆ alkyl), —SO₂N(C₁-C₆ alkyl)₂, —C(═NH)NH₂, and —NO₂, in certain embodiments containing one or two substituents independently selected from halogen, —OH, alkoxy, —NH₂, trifluoromethyl, —N(CH₃)₂, and —C(═O)OH, in certain embodiments independently selected from halogen, alkoxy and —OH. Examples of substituted alkyls include, but are not limited to, 2,2-difluoropropyl, 2-carboxycyclopentyl and 3-chloropropyl.

For aryl, aryl-(C₁-C₃)alkyl and heterocyclyl groups, the term “substituted” as applied to the rings of these groups refers to any level of substitution, namely mono-, di-, tri-, tetra-, or penta-substitution, where such substitution is permitted. The substituents are independently selected, and substitution may be at any chemically accessible position. In certain embodiments, the substituents vary in number between one and four. In other embodiments, the substituents vary in number between one and three. In yet another embodiments, the substituents vary in number between one and two. In yet other embodiments, the substituents are independently selected from the group consisting of C₁-C₆ alkyl, —OH, C₁-C₆ alkoxy, halo, amino, acetamido and nitro. As used herein, where a substituent is an alkyl or alkoxy group, the carbon chain may be branched, straight or cyclic.

Unless otherwise noted, when two substituents are taken together to form a ring having a specified number of ring atoms (e.g., R² and R³ taken together with the nitrogen to which they are attached to form a ring having from 3 to 7 ring members), the ring can have carbon atoms and optionally one or more (e.g., 1 to 3) additional heteroatoms independently selected from nitrogen, oxygen, or sulfur. The ring can be saturated or partially saturated, and can be optionally substituted.

Whenever a term or either of their prefix roots appear in a name of a substituent the name is to be interpreted as including those limitations provided herein. For example, whenever the term “alkyl” or “aryl” or either of their prefix roots appear in a name of a substituent (e.g., arylalkyl, alkylamino) the name is to be interpreted as including those limitations given elsewhere herein for “alkyl” and “aryl” respectively.

In certain embodiments, substituents of compounds are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

The terms “treat,” “treating” and “treatment,” as used herein, means reducing the frequency or severity with which symptoms of a disease or condition are experienced by a subject by virtue of administering an agent or compound to the subject.

Certain abbreviations used herein follow: cccDNA, covalently closed circular DNA; DAD, diode array detector; DCC, N,N′-Dicyclohexylcarbodiimide; DCE, 1,2-dichloroethane; DCM, dichloromethane; DIEA or DIPEA, diisopropylethylamine; DMF, N,N-dimethylformamide; DMSO, dimethylsulfoxide; DPPA, Diphenylphosphoryl azide; EDCI or EDC, 1-Ethyl-3-(3-dimethylaminopropyl)carbodiimide hydrochloride; EtOAc, ethyl acetate; HATU, hexafluorophosphate azabenzotriazole tetramethyl uronium; HBsAg, HBV surface antigen; HBV, hepatitis B virus; HDV, hepatitis D virus; HOBt, Hydroxybenzotriazole; HPLC, high-performance liquid chromatography; IPA, isopropanol (2-propanol); LCMS, liquid chromatography mass spectrometry; LG, leaving group; m-CPBA, meta-chloroperbenzoic acid; NARTI or NRTI, reverse-transcriptase inhibitor; NMR, Nuclear Magnetic Resonance; NtARTI or NtRTI, nucleotide analog reverse-transcriptase inhibitor; pg RNA, pregenomic RNA; rcDNA, relaxed circular DNA; RT, retention time; sAg, surface antigen; SFC, supercritical fluid chromatography; STAB, sodium triacetoxyborohydride; TBAF, tetrabutyl ammonium fluoride; TFA, trifluoroacetic acid; THF, tetrahydrofuran; TLC, thin layer chromatography; TMSOTf, trimethylsilyl trifluoromethylsulfonate; UPLC, Ultra-performance liquid chromatography.

Ranges: throughout this disclosure, various aspects of the present disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise. This applies regardless of the breadth of the range.

### Compounds

The disclosure includes a compound of formula (I), or a salt, solvate, prodrug, isotopically labeled derivative, stereoisomer (such as, in a non-limiting example, an enantiomer or diastereoisomer, and/or any mixtures thereof, such as, in a non-limiting example, mixtures in any proportions of enantiomers and/or diastereoisomers thereof), tautomer and any mixtures thereof, and/or geometric isomer and any mixtures thereof:

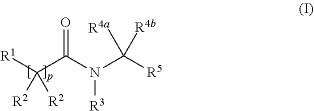

wherein:
R¹ is selected from the group consisting of

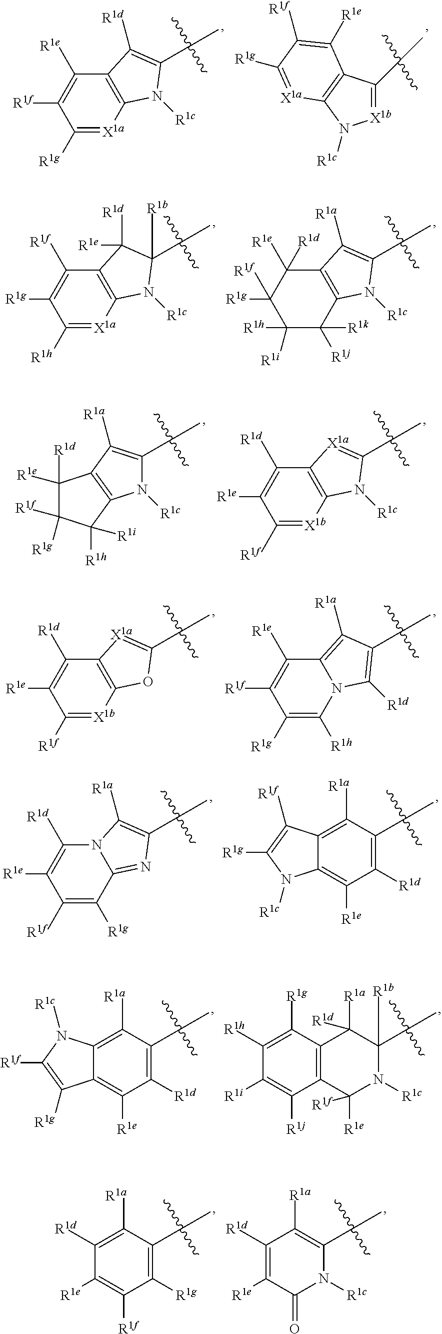

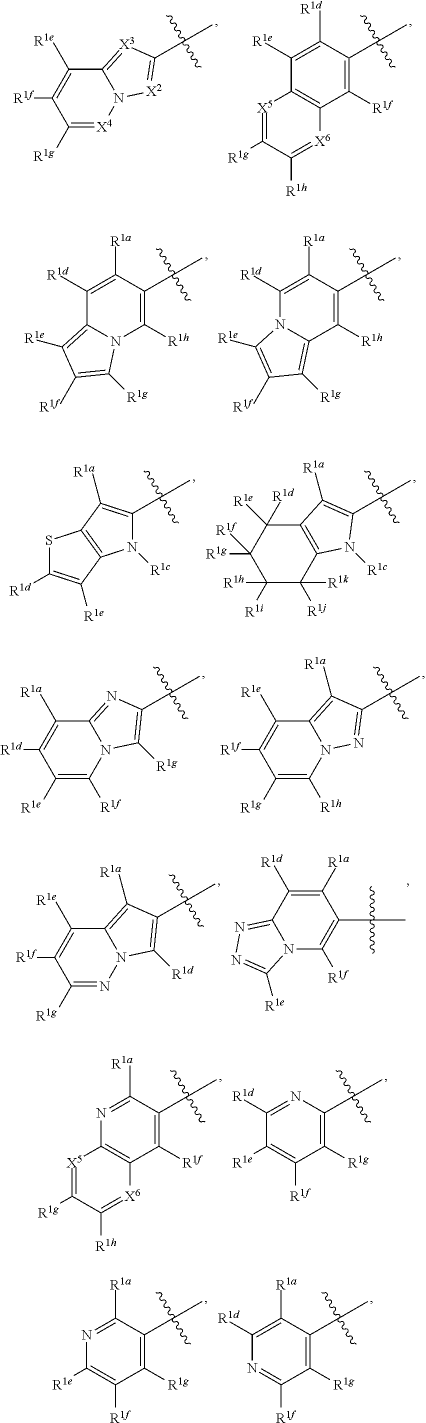

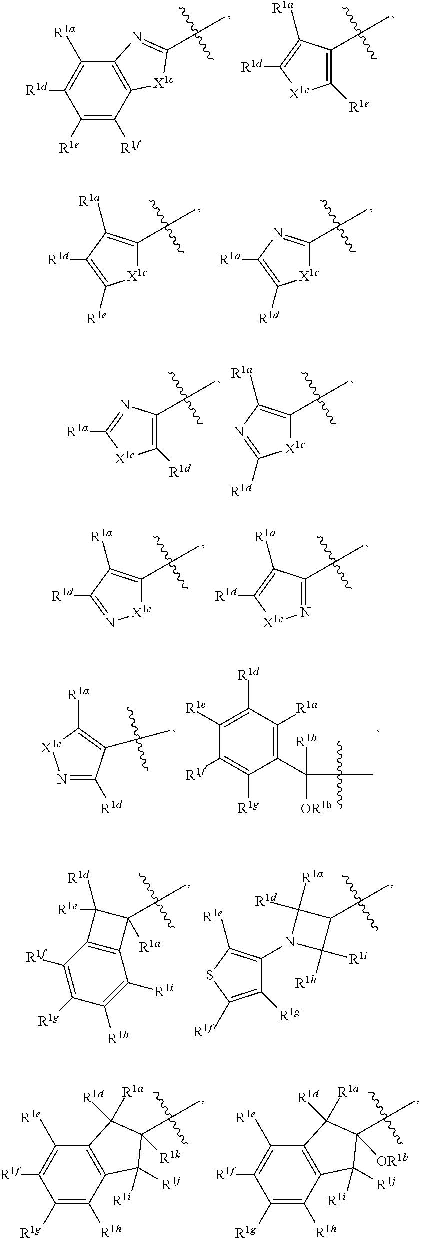

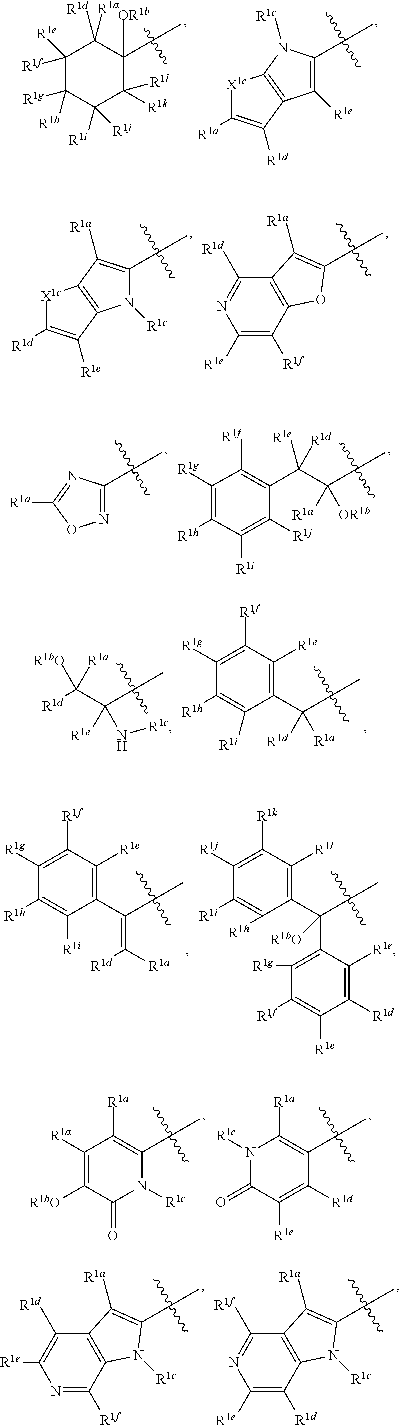

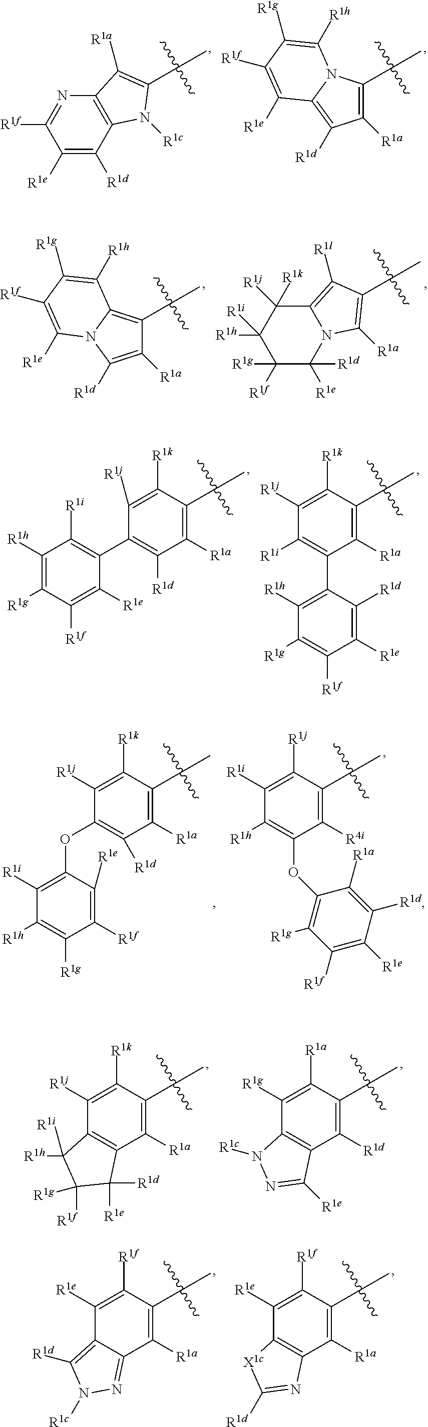

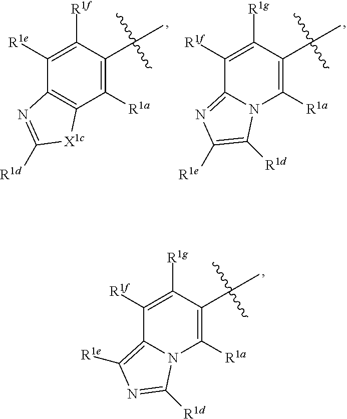

optionally substituted C₃-C₈ cycloalkyl, —NH(optionally substituted C₃-C₈ cycloalkyl), and —NH(optionally substituted phenyl);
each occurrence of R^{1a}, R^{1d}, R^{1e}, R^{1f}, R^{1g}, R^{1h}, R¹ⁱ, R^{1j}, R^{1k} and R^{1l} is independently selected from the group consisting of H, halogen, —CN, optionally substituted C₁-C₆ alkyl, optionally substituted C₃-C₈ cycloalkyl, optionally substituted C₁-C₆ alkoxy, optionally substituted C₃-C₈ cycloalkoxy, heterocyclyl, heteroaryl, —S(optionally substituted C₁-C₆ alkyl), —SO(optionally substituted C₁-C₆ alkyl), —SO₂(optionally substituted C₁-C₆ alkoxy), —C(═O)OH, —C(═O)O(optionally substituted C₁-C₆ alkyl), —C(═O)O(optionally substituted C₃-C₈ cycloalkyl), —O(optionally substituted C₁-C₆ alkyl), —O(optionally substituted C₃-C₈ cycloalkyl), —NH₂, —NH(optionally substituted C₁-C₆ alkyl), —NH(optionally substituted C₃-C₈ cycloalkyl), —N(optionally substituted C₁-C₆ alkyl)(optionally substituted C₁-C₆ alkyl), —N(optionally substituted C₃-C₈ cycloalkyl)(optionally substituted C₃-C₈ cycloalkyl), —N(optionally substituted C₁-C₆ alkyl)(optionally substituted C₃-C₈ cycloalkyl), —C(═O)NH₂, —C(═O)NH(optionally substituted C₁-C₆ alkyl), —C(═O)NH(optionally substituted C₃-C₈ cycloalkyl), —C(═O)N(optionally substituted C₁-C₆ alkyl)(optionally substituted C₁-C₆ alkyl), —C(═O)N(optionally substituted C₃-C₈ cycloalkyl)(optionally substituted C₃-C₈ cycloalkyl), and —C(═O)N(optionally substituted C₁-C₆ alkyl)(optionally substituted C₃-C₈ cycloalkyl;
each occurrence of R^{1b} is independently H, optionally substituted C₁-C₆ alkyl, or optionally substituted C₃-C₈ cycloalkyl;
each occurrence of R^{1c} is independently H, optionally substituted C₁-C₆ alkyl, or optionally substituted C₃-C₈ cycloalkyl;
each occurrence of X^{1a} is independently N or C(R^{1a});
each occurrence of X^{1b} is independently N or C(R^{1d})
each occurrence of X^{1c} is independently N(R^{1b}), O, or S;
one of the following applies:
   (i) X² is N, X³ is C(R^{1a}), and X⁴ is C(R^{1d});
   (ii) X³ is N, X² is C(R^{1a}), and X⁴ is C(R^{1d}); or
   (iii) X⁴ is N, X² is C(R^{1a}), and X³ is C(R^{1d});
one of the following applies:
   (i) X⁵ is N and X⁶ is C(R^{1a}); or
   (ii) X⁶ is N and X⁵ is C(R^{1a});
each occurrence of R² is independently H, optionally substituted C₁-C₆ alkyl, or optionally substituted C₃-C₈ cycloalkyl, or the two R² groups combine to form ═O;
R³ is selected from the group consisting of H, C₁-C₆ alkyl, and C₃-C₈ cycloalkyl, wherein the alkyl or cycloalkyl is optionally substituted with at least one substituent selected from the group consisting of C₁-C₆ alkyl, C₃-C₈ cycloalkyl, halogen, cyano, —OH, C₁-C₆ alkoxy, C₃-C₈ cycloalkoxy, C₁-C₆ haloalkoxy, C₃-C₈ halocycloalkoxy, optionally substituted phenyl, optionally substituted heteroaryl, optionally substituted heterocyclyl, —C(═O)OR⁶, —OC(═O)R⁶, —SR⁶, —S(═O)R⁶, —S(═O)₂R⁶, —S(═O)₂NR⁶R⁶, —N(R⁶)S(═O)₂R⁶, —N(R⁶)C(═O)R⁶, —C(═O)NR⁶R⁶, and —NR⁶R⁶;
R^{4a} is selected from the group consisting of H, C₁-C₆ alkyl, C₃-C₈ cycloalkyl, and phenyl, wherein the alkyl, cycloalkyl, or phenyl is optionally substituted with at least one substituent selected from the group consisting of C₁-C₆ alkyl, C₃-C₈ cycloalkyl, halogen, cyano, —OH, C₁-C₆ alkoxy, C₃-C₈ cycloalkoxy, C₁-C₆ haloalkoxy, C₃-C₈ halocycloalkoxy, —NR⁶R⁶, and optionally substituted phenyl;
R^{4b} selected from the group consisting of H and optionally substituted C₁-C₆ alkyl;
R⁵ is selected from the group consisting of:

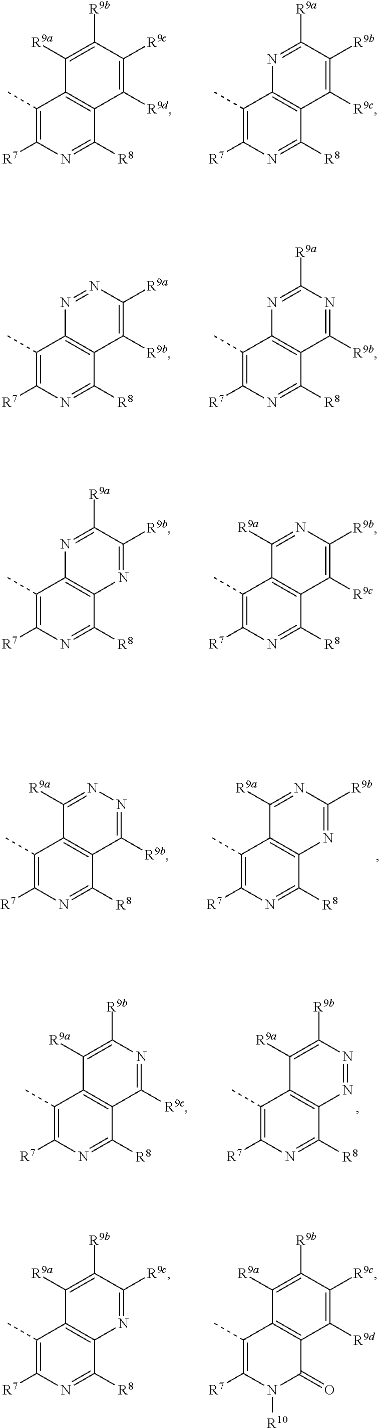

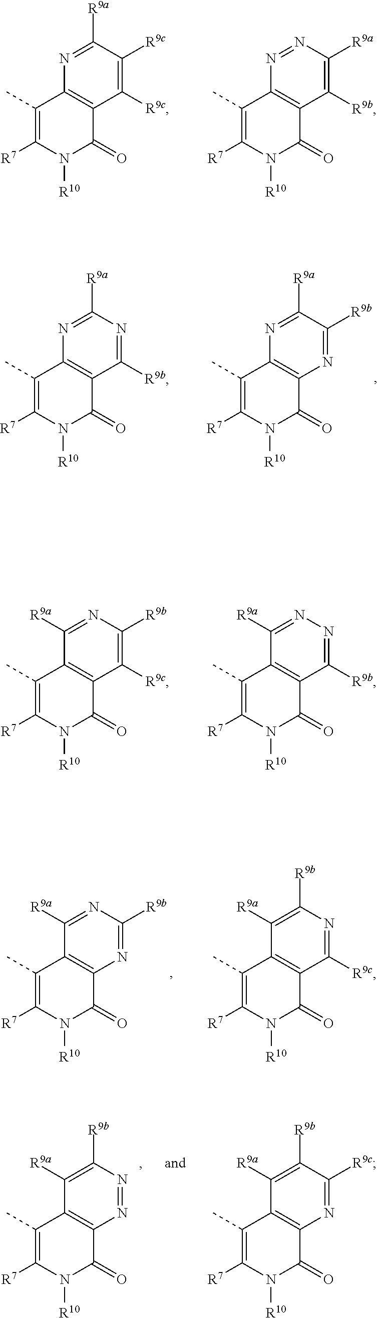

each occurrence of R⁶ is independently selected from the group consisting of H, optionally substituted C₁-C₆ alkyl, optionally substituted C₃-C₈ cycloalkyl, optionally substituted phenyl, and optionally substituted hetereoaryl;
each occurrence of R⁷ is independently selected from the group consisting of H, halogen, optionally substituted C₁-C₆ alkyl, optionally substituted C₃-C₈ cycloalkyl, optionally substituted C₁-C₆ alkoxy, and optionally substituted C₃-C₈ cycloalkoxy;
each occurrence of R⁸ is independently selected from the group consisting of H, halogen, —CN, optionally substituted C₁-C₆ alkyl, optionally substituted C₃-C₈ cycloalkyl, optionally substituted C₁-C₆ alkoxy, optionally substituted C₃-C₈ cycloalkoxy, heterocyclyl, heteroaryl, —S(optionally substituted C₁-C₆ alkyl), —SO(optionally substituted C₁-C₆ alkyl), —SO₂(optionally substituted C₁-C₆ alkyl), —C(═O)OH, —C(═O)O(optionally substituted C₁-C₆ alkyl), —C(═O)O(optionally substituted C₃-C₈ cycloalkyl), —O(optionally substituted C₁-C₆ alkyl), —O(optionally substituted C₃-C₈ cycloalkyl), —NH₂, —NH(optionally substituted C₁-C₆ alkyl), —NH(optionally substituted C₃-C₈ cycloalkyl), —N(optionally substituted C₁-C₆ alkyl)(optionally substituted C₁-C₆ alkyl), —N(optionally substituted C₃-C₈ cycloalkyl)(optionally substituted C₃-C₈ cycloalkyl), —N(optionally substituted C₁-C₆ alkyl)(optionally substituted C₃-C₈ cycloalkyl), —C(═O)NH₂, —C(═O)NH(optionally substituted C₁-C₆ alkyl), —C(═O)NH(optionally substituted C₃-C₈ cycloalkyl), —C(═O)N(optionally substituted C₁-C₆ alkyl)(optionally substituted C₁-C₆ alkyl), —C(═O)N(optionally substituted C₃-C₈ cycloalkyl)(optionally substituted C₃-C₈ cycloalkyl), and —C(═O)N(optionally substituted C₁-C₆ alkyl)(optionally substituted C₃-C₈ cycloalkyl;
each occurrence of R^{9a}, R^{9b}, R^{9c}, and R^{9d} is independently selected from the group consisting of H, halogen, optionally substituted C₁-C₆ alkyl, optionally substituted C₃-C₈ cycloalkyl, optionally substituted C₁-C₆ alkoxy, and optionally substituted C₃-C₈ cycloalkoxy;
each occurrence of R¹⁰ is independently selected from the group consisting of H, optionally substituted C₁-C₆ alkyl, and optionally substituted C₃-C₈ cycloalkyl; and
each occurrence of p is 0 or 1,
   wherein, if p is 0, then R¹ is not —NH(optionally substituted C₃-C₈ cycloalkyl) or —NH(optionally substituted phenyl).

In certain embodiments, the compound of formula (I) is a compound of formula (Ia):

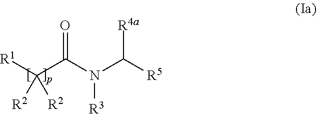

In certain embodiments, the compound of formula (I) is selected from:

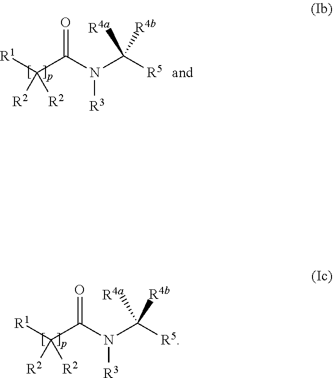

In certain embodiments, the compound of formula (I) is selected from:

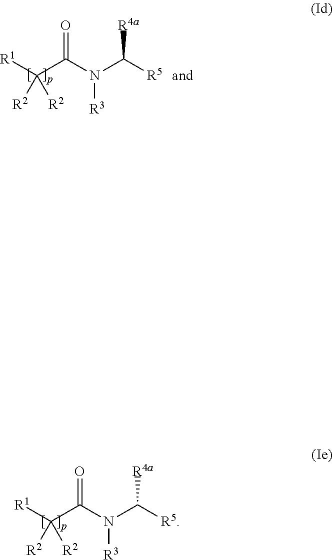

In certain embodiments, the compound of formula (I) is a compound of formula (If):

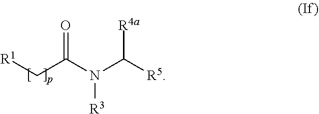

In certain embodiments, the compound of formula (I) is selected from:

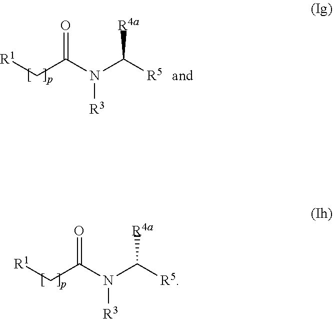

In certain embodiments, each occurrence of alkyl, alkenyl, alkynyl, or cycloalkyl is independently optionally substituted with at least one substituent selected from the group consisting of C₁-C₆ alkyl, C₃-C₈ cycloalkyl, halo, cyano (—CN), —OR^{a}, optionally substituted phenyl (thus yielding, in non-limiting examples, optionally substituted phenyl-(C₁-C₃ alkyl), such as, but not limited to, benzyl or substituted benzyl), optionally substituted heteroaryl, optionally substituted heterocyclyl, —C(═O)OR^{a}, —OC(═O)R^{a}, —SR^{a}, —S(═O)R^{a}, —S(═O)₂R^{a}, —S(═O)₂NR^{a}R^{a}, —N(R^{a})S(═O)₂R^{a}, —N(R^{a})C(═O)R^{a}, —C(═O)NR^{a}R^{a}, and —N(R^{a})(R^{a}), wherein each occurrence of R^{a} is independently H, optionally substituted C₁-C₆ alkyl, optionally substituted C₃-C₈ cycloalkyl, optionally substituted aryl, or optionally substituted heteroaryl, or two R^{a} groups combine with the N to which they are bound to form a heterocycle.

In certain embodiments, each occurrence of aryl or heteroaryl is independently optionally substituted with at least one substituent selected from the group consisting of C₁-C₆ alkyl, C₃-C₈ cycloalkyl, phenyl, C₁-C₆ hydroxyalkyl, (C₁-C₆ alkoxy)-C₁-C₆ alkyl, C₁-C₆ haloalkyl, C₁-C₆ haloalkoxy, halogen, —CN, —OR^{b}, —N(R^{b})(R^{b}), —NO₂, —C(═O)N(R^{b})(R^{b}), —C(═O)OR^{b}, —OC(═O)R^{b}, —SR^{b}, —S(═O)R^{b}, —S(═O)₂R^{b}, —N(R)S(═O)₂R^{b}, —S(═O)₂N(R^{b})(R^{b}), acyl, and C₁-C₆ alkoxycarbonyl, wherein each occurrence of R^{b} is independently H, C₁-C₆ alkyl, or C₃-C₈ cycloalkyl, wherein in R the alkyl or cycloalkyl is optionally substituted with at least one selected from the group consisting of halogen, —OH, C₁-C₆ alkoxy, and heteroaryl; or substituents on two adjacent carbon atoms combine to form —O(CH₂)₁₋₃O—.

In certain embodiments, each occurrence of aryl or heteroaryl is independently optionally substituted with at least one substituent selected from the group consisting of C₁-C₆ alkyl, C₃-C₈ cycloalkyl, phenyl, C₁-C₆ hydroxyalkyl, (C₁-C₆ alkoxy)-C₁-C₆ alkyl, C₁-C₆ haloalkyl, C₁-C₆ haloalkoxy, halogen, —OR^{b}, —C(═O)N(R^{b})(R^{b}), —C(═O)OR^{b}, —OC(═O)R^{b}, —SR, —S(═O)R, —S(═O)₂R^{b}, and —N(R^{b})S(═O)₂R^{b}, wherein each occurrence of R^{b} is independently H, C₁-C₆ alkyl, or C₃-C₈ cycloalkyl, wherein in R^{b} the alkyl or cycloalkyl is optionally substituted with at least one selected from the group consisting of halogen, —OH, C₁-C₆ alkoxy, and heteroaryl; or substituents on two adjacent carbon atoms combine to form —O(CH₂)₁₋₃O—.

In certain embodiments, the alkyl, alkenyl, alkynyl, cycloalkyl, heteroaryl, heterocyclyl, aryl, or benzyl group is optionally independently substituted with at least one group selected from the group consisting of C₁-C₆ alkyl; C₁-C₆ alkoxy; C₁-C₆ haloalkyl; C₁-C₆ haloalkoxy; —NH₂, —NH(C₁-C₆ alkyl), —N(C₁-C₆ alkyl)(C₁-C₆ alkyl), halogen, —OH; —CN; phenoxy, —NHC(═O)H, —NHC(═O)C₁-C₆ alkyl, —C(═O)NH₂, —C(═O)NHCl—C₆ alkyl, —C(═O)N(C₁-C₆ alkyl)(C₁-C₆ alkyl), tetrahydropyranyl, morpholinyl, —C(═O)CH₃, —C(═O)CH₂OH, —C(═O)NHCH₃, —C(═O)CH₂OMe, or an N-oxide thereof.

In certain embodiments, each occurrence of the heteroaryl is independently selected from the group consisting of quinolinyl, imidazo[1,2-a]pyridyl, pyridyl, pyrimidyl, pyrazinyl, imidazolyl, thiazolyl, pyrazolyl, isoxazolyl, oxadiazolyl (including 1,2,3-, 1,2,4-, 1,2,5-, and 1,3,4-oxadiazole), and triazolyl (such as 1,2,3-triazolyl and 1,2,4-triazolyl).

In certain embodiments, each occurrence of the heterocyclyl group is independently selected from the group consisting of tetrahydrofuranyl, tetrahydropyranyl, piperidinyl, piperazinyl, pyrrolidinyl, morpholinyl, thiomorpholinyl, 1-oxido-thiomorpholinyl, 1,1-dioxido-thiomorpholinyl, oxazolidinyl, azetidinyl, and the corresponding oxo analogues (where a methylene ring group is replaced with a carbonyl) thereof.

In certain embodiments, R¹ is

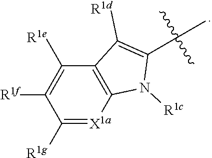

In certain embodiments, R¹ is

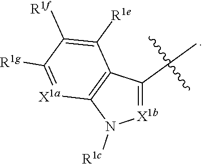

In certain embodiments, R¹ is

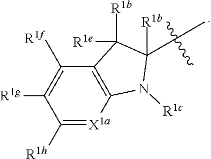

In certain embodiments, R¹ is

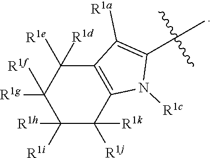

In certain embodiments, R¹ is

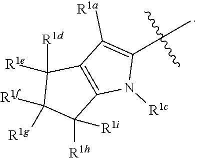

In certain embodiments, R¹ is

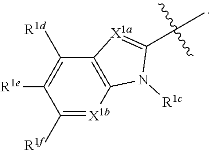

In certain embodiments, R¹ is

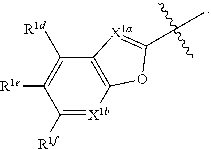

In certain embodiments, R¹ is

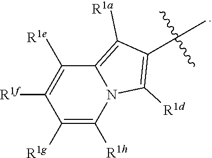

In certain embodiments, R¹ is

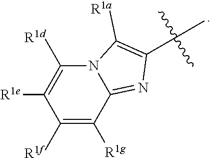

In certain embodiments, R¹ is

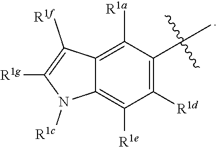

In certain embodiments, R¹ is

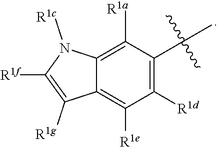

In certain embodiments, R¹ is

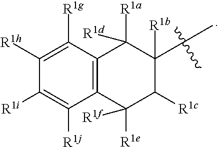

In certain embodiments, R¹ is

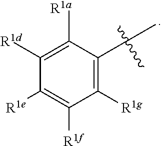

In certain embodiments, R¹ is

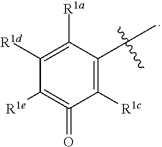

In certain embodiments, R¹ is

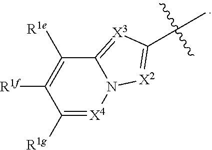

In certain embodiments, R¹ is

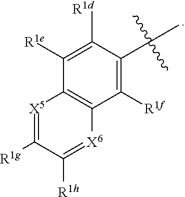

In certain embodiments, R¹ is

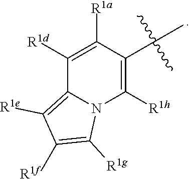

In certain embodiments, R¹ is

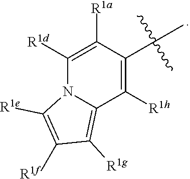

In certain embodiments, R¹ is

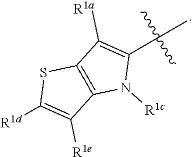

In certain embodiments, R¹ is

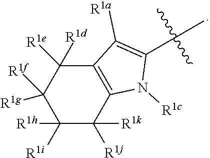

In certain embodiments, R¹ is

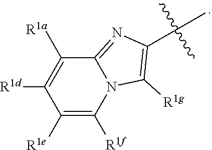

In certain embodiments, R¹ is

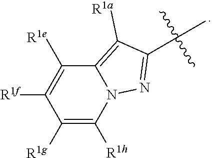

In certain embodiments, R¹ is

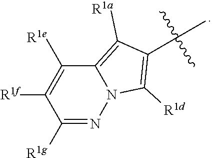

In certain embodiments, R¹ is

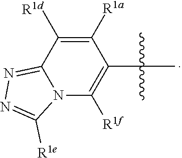

In certain embodiments, R¹ is

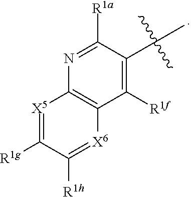

In certain embodiments, R¹ is

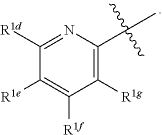

In certain embodiments, R¹ is

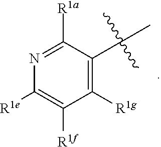

In certain embodiments, R¹ is

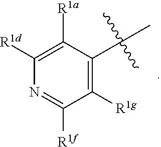

In certain embodiments, R¹ is

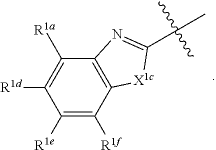

In certain embodiments, R¹ is

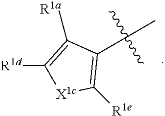

In certain embodiments, R¹ is

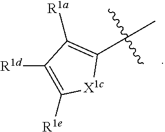

In certain embodiments, R¹ is

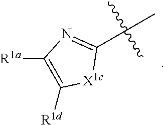

In certain embodiments, R¹ is

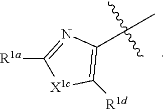

In certain embodiments, R¹ is

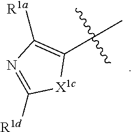

In certain embodiments, R¹ is

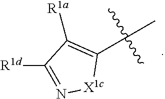

In certain embodiments, R¹

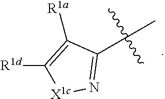

In certain embodiments, R¹ is

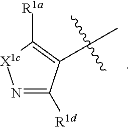

In certain embodiments, R¹ is

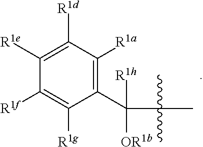

In certain embodiments, R¹ is

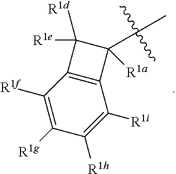

In certain embodiments, R¹ is

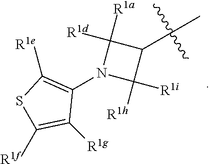

In certain embodiments, R¹ is

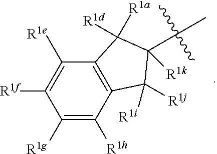

In certain embodiments, R¹ is

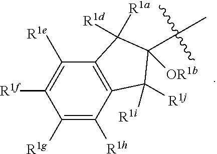

In certain embodiments, R¹ is

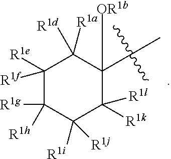

In certain embodiments, R¹ is

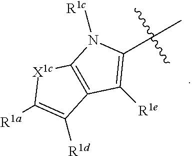

In certain embodiments, R¹ is

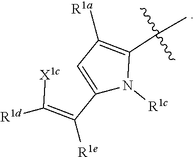

In certain embodiments, R¹ is

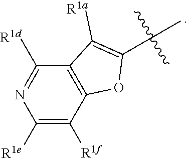

In certain embodiments, R¹ is

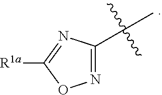

In certain embodiments, R¹ is

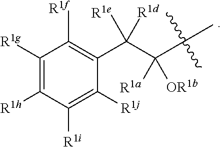

In certain embodiments, R¹ is

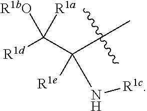

In certain embodiments, R¹ is

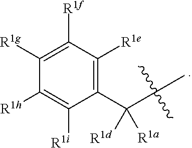

In certain embodiments, R¹ is

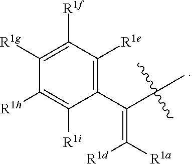

In certain embodiments, R¹ is

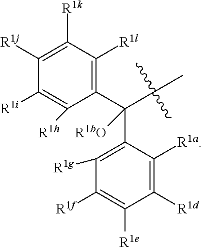

In certain embodiments, R¹ is

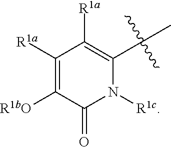

In certain embodiments, R¹ is

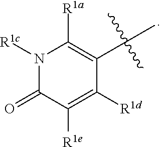

In certain embodiments, R¹ is

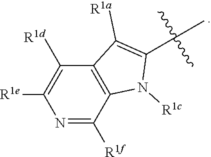

In certain embodiments, R¹ is

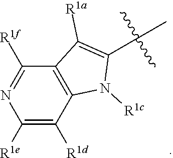

In certain embodiments, R¹ is

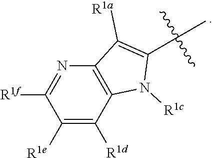

In certain embodiments, R¹ is

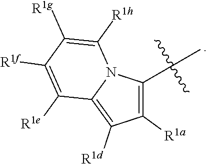

In certain embodiments, R¹ is

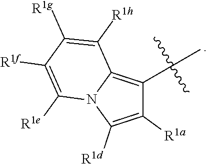

In certain embodiments, R¹ is

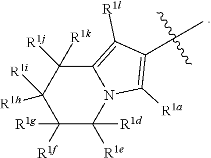

In certain embodiments, R¹ is

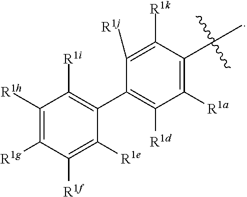

In certain embodiments, R¹ is

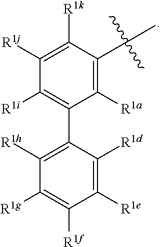

In certain embodiments, R¹ is

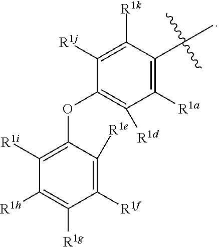

In certain embodiments, R¹ is

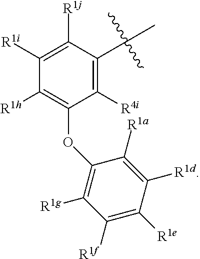

In certain embodiments, R¹ is

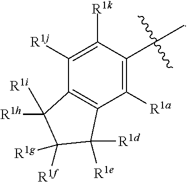

In certain embodiments, R¹ is

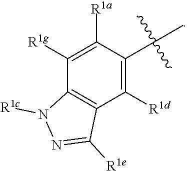

In certain embodiments, R¹ is

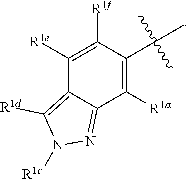

In certain embodiments, R¹ is

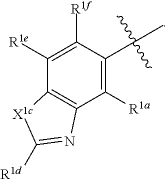

In certain embodiments, R¹ is

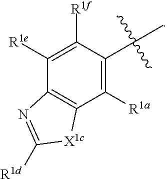

In certain embodiments, R¹ is

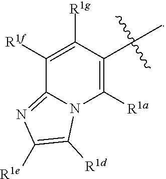

In certain embodiments, R¹ is

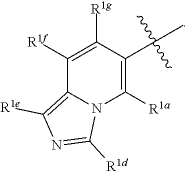

In certain embodiments, R¹ is optionally substituted C₃-C₈ cycloalkyl. In certain embodiments, R¹ is NH(optionally substituted C₃-C₈ cycloalkyl). In certain embodiments, R¹ is —NH(optionally substituted phenyl)

In certain embodiments, R¹ is phenyl optionally substituted with at least one selected from the group consisting of C₁-C₆ alkyl (such as, for example, methyl, ethyl, and isopropyl), halo (such as, for example, F, Cl, Br, and I), C₁-C₃ haloalkyl (such as, for example, monofluoromethyl, difluoromethyl, and trifluoromethyl), and CN.

In certain embodiments, R¹ is selected from the group consisting of: phenyl, 3-chlorophenyl, 3-fluorophenyl, 4-fluorophenyl, 3,5-difluorophenyl, 2,4,5-trifluorophenyl, 3,4-dichlorophenyl, 4-chloro-3-methylphenyl, 3-chloro-4-methylphenyl, 4-fluoro-3-methylphenyl, 3-fluoro-4-methylphenyl, 4-chloro-3-methoxyphenyl, 3-chloro-4-methoxyphenyl, 4-fluoro-3-methoxyphenyl, 3-fluoro-4-methoxyphenyl, 3-trifluoromethylphenyl, 3-trifluoromethyl-4-fluorophenyl, 3-cyanophenyl, 4-cyanophenyl, 3-cyano-4-fluorophenyl, 4-cyano-3-fluorophenyl, 4-difluoromethyl-3-fluorophenyl, benzo[d][1,3]dioxol-5-yl, 2,3-dihydrobenzo[b][1,4]dioxin-6-yl, benzyl, 3-fluorobenzyl, 4-fluorobenzyl, 3-chlorobenzyl, 4-chlorobenzyl, 2-pyridyl, 4-methyl-2-pyridyl, 5-methyl-2-pyridyl, 6-methyl-2-pyridyl, 3-pyridyl, 2-methyl-3-pyridyl, 3-methyl-3-pyridyl, 4-pyridyl, 2-methyl-4-pyridyl, and 6-methyl-4-pyridyl.

In yet other embodiments, R¹ is 4-fluoro-3-methylphenyl. In yet other embodiments, R¹ is 3-cyano-4-fluorophenyl. In yet other embodiments, R¹ is 4-difluoromethyl-3-fluorophenyl. In certain embodiments, R¹ is

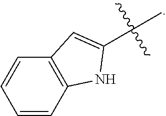 [SMILES: *c1cc2ccccc2[nH]1]

In certain embodiments, R¹ is

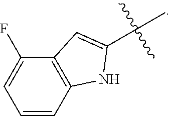 [SMILES: *c1cc2c(F)cccc2[nH]1]

In certain embodiments, R¹

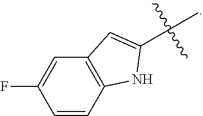 [SMILES: *c1cc2cc(F)ccc2[nH]1]

In certain embodiments, R¹ is

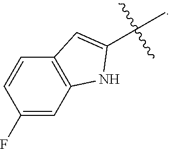 [SMILES: *c1cc2ccc(F)cc2[nH]1]

In certain embodiments, R¹ is

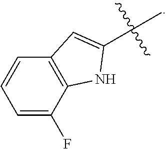 [SMILES: *c1cc2cccc(F)c2[nH]1]

In certain embodiments, R¹ is

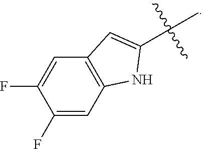 [SMILES: *c1cc2cc(F)c(F)cc2[nH]1]

In certain embodiments, R¹ is

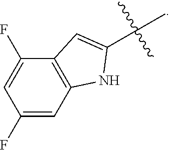 [SMILES: *c1cc2c(F)cc(F)cc2[nH]1]

In certain embodiments, R¹ is

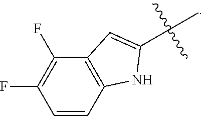 [SMILES: *c1cc2c(F)c(F)ccc2[nH]1]

In certain embodiments, R¹ is

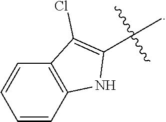 [SMILES: *c1[nH]c2ccccc2c1Cl]

In certain embodiments, R¹ is

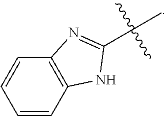 [SMILES: *c1nc2ccccc2[nH]1]

In certain embodiments, R¹ is

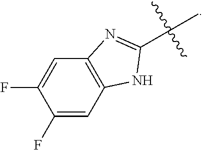 [SMILES: *c1nc2cc(F)c(F)cc2[nH]1]

In certain embodiments, R¹ is

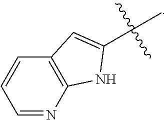 [SMILES: *c1cc2cccnc2[nH]1]

In certain embodiments, R¹ is

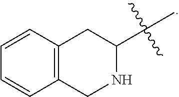 [SMILES: *C1Cc2ccccc2CN1]

In certain embodiments, R¹ is

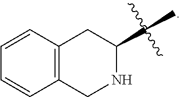 [SMILES: *[C@@H]1Cc2ccccc2CN1]

In certain embodiments, R¹ is

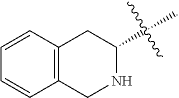 [SMILES: *[C@H]1Cc2ccccc2CN1]

In certain embodiments, R¹ is

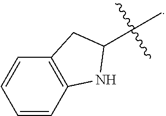 [SMILES: *C1Cc2ccccc2N1]

In certain embodiments, R¹ is

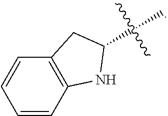 [SMILES: *[C@H]1Cc2ccccc2N1]

In certain embodiments, R¹ is

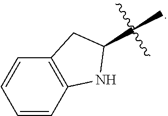 [SMILES: *[C@@H]1Cc2ccccc2N1]

In certain embodiments, R¹ is

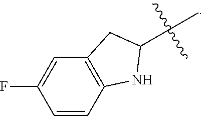 [SMILES: *C1Cc2cc(F)ccc2N1]

In certain embodiments, R¹ is

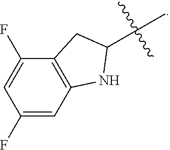 [SMILES: *C1Cc2c(F)cc(F)cc2N1]

In certain embodiments, R¹ is

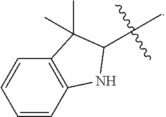 [SMILES: *C1Nc2ccccc2C1(C)C]

In certain embodiments, R¹ is

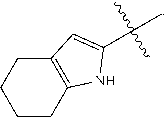 [SMILES: *c1cc2c([nH]1)CCCC2]

In certain embodiments, R¹ is

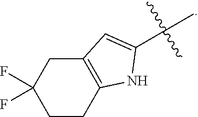 [SMILES: *c1cc2c([nH]1)CCC(F)(F)C2]

In certain embodiments, R¹ is

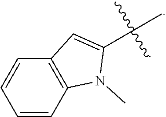 [SMILES: *c1cc2ccccc2n1C]

In certain embodiments, R¹ is

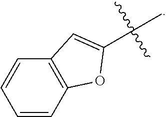 [SMILES: *c1cc2ccccc2o1]

In certain embodiments, R¹ is

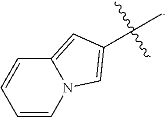 [SMILES: *c1cc2ccccn2c1]

In certain embodiments, R¹ is

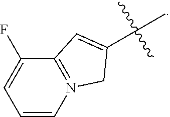

In certain embodiments, R¹ is

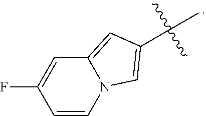 [SMILES: *c1cc2cc(F)ccn2c1]

In certain embodiments, R¹ is

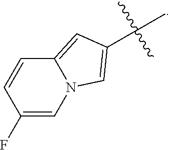 [SMILES: *c1cc2ccc(F)cn2c1]

In certain embodiments, R¹ is

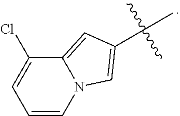 [SMILES: *c1cc2c(Cl)cccn2c1]

In certain embodiments, R¹ is

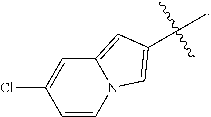 [SMILES: *c1cc2cc(Cl)ccn2c1]

In certain embodiments, R¹ is

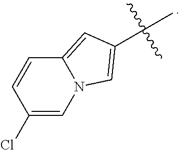 [SMILES: *c1cc2ccc(Cl)cn2c1]

In certain embodiments, R¹ is

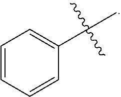 [SMILES: *c1ccccc1]

In certain embodiments, R¹ is

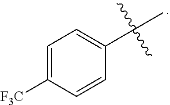 [SMILES: *c1ccc(C(F)(F)F)cc1]

In certain embodiments, R¹ is

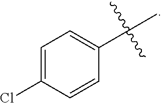 [SMILES: *c1ccc(Cl)cc1]

In certain embodiments, R¹ is

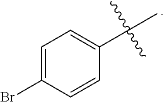 [SMILES: *c1ccc(Br)cc1]

In certain embodiments, R¹ is

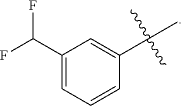 [SMILES: *c1cccc(C(F)F)c1]

In certain embodiments, R¹ is

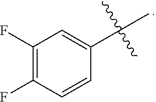 [SMILES: *c1ccc(F)c(F)c1]

In certain embodiments, R¹ is

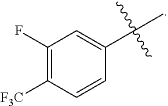 [SMILES: *c1ccc(C(F)(F)F)c(F)c1]

In certain embodiments, R¹ is

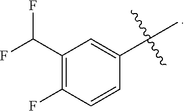 [SMILES: *c1ccc(F)c(C(F)F)c1]

In certain embodiments, R¹ is

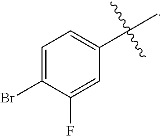 [SMILES: *c1ccc(Br)c(F)c1]

In certain embodiments, R¹ is

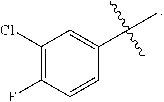 [SMILES: *c1ccc(F)c(Cl)c1]

In certain embodiments, R¹ is

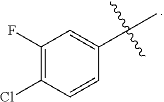 [SMILES: *c1ccc(Cl)c(F)c1]

In certain embodiments, R¹ is

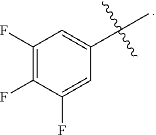 [SMILES: *c1cc(F)c(F)c(F)c1]

In certain embodiments, R¹ is

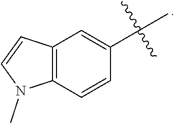 [SMILES: *c1ccc2c(ccn2C)c1]

In certain embodiments, R¹ is

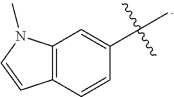 [SMILES: *c1ccc2ccn(C)c2c1]

In certain embodiments, R¹ is

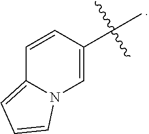 [SMILES: *c1ccc2cccn2c1]

In certain embodiments, R¹ is

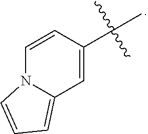 [SMILES: *c1ccn2cccc2c1]

In certain embodiments, R¹ is

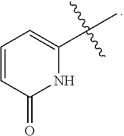 [SMILES: *c1cccc(=O)[nH]1]

In certain embodiments, R¹ is

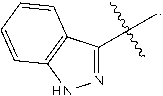 [SMILES: *c1n[nH]c2ccccc12]

In certain embodiments, R¹ is

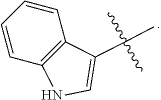 [SMILES: *c1c[nH]c2ccccc12]

In certain embodiments, R¹ is

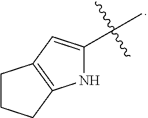 [SMILES: *c1cc2c([nH]1)CCC2]

In certain embodiments, R¹ is

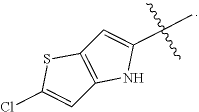 [SMILES: *c1cc2sc(Cl)cc2[nH]1]

In certain embodiments, R¹ is

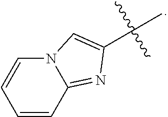 [SMILES: *c1cn2ccccc2n1]

In certain embodiments, R¹ is cyclopropyl. In certain embodiments, R¹ is

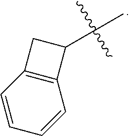 [SMILES: *C1Cc2ccccc21]

In certain embodiments, R¹ is

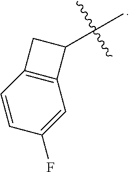 [SMILES: *C1Cc2ccc(F)cc21]

In certain embodiments, R¹ is

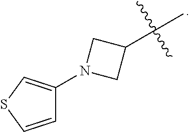 [SMILES: *C1CN(c2ccsc2)C1]

In certain embodiments, R¹ is

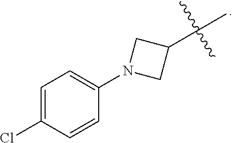 [SMILES: *C1CN(c2ccc(Cl)cc2)C1]

In certain embodiments, R¹ is

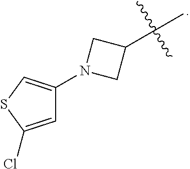 [SMILES: *C1CN(c2csc(Cl)c2)C1]

In certain embodiments, R¹ is

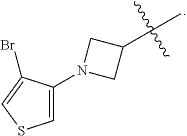 [SMILES: *C1CN(c2cscc2Br)C1]

In certain embodiments, R¹ is

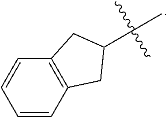 [SMILES: *C1Cc2ccccc2C1]

In certain embodiments, R¹ is

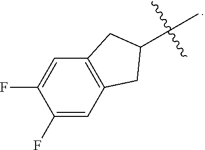 [SMILES: *C1Cc2cc(F)c(F)cc2C1]

In certain embodiments, R¹ is

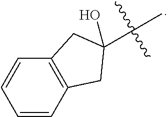 [SMILES: *C1(O)Cc2ccccc2C1]

In certain embodiments, R¹ is

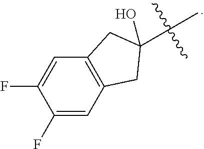 [SMILES: *C1(O)Cc2cc(F)c(F)cc2C1]

In certain embodiments, R¹ is

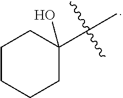 [SMILES: *C1(O)CCCCC1]

In certain embodiments, R¹ is

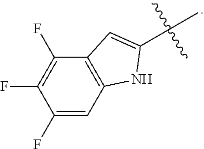 [SMILES: *c1cc2c(F)c(F)c(F)cc2[nH]1]

In certain embodiments, R¹ is

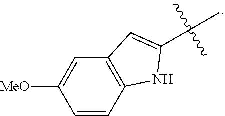 [SMILES: *c1cc2cc(OC)ccc2[nH]1]

In certain embodiments, R¹ is

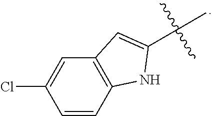 [SMILES: *c1cc2cc(Cl)ccc2[nH]1]

In certain embodiments, R¹ is

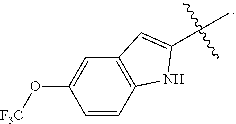 [SMILES: *c1cc2cc(OC(F)(F)F)ccc2[nH]1]

In certain embodiments, R¹ is

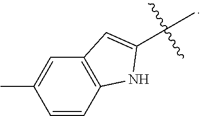 [SMILES: *c1cc2cc(C)ccc2[nH]1]

In certain embodiments, R¹ is

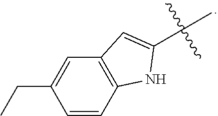 [SMILES: *c1cc2cc(CC)ccc2[nH]1]

In certain embodiments, R¹ is

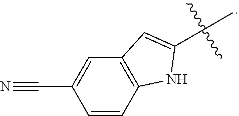 [SMILES: *c1cc2cc(C#N)ccc2[nH]1]

In certain embodiments, R¹ is

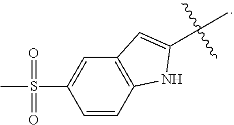 [SMILES: *c1cc2cc(S(C)(=O)=O)ccc2[nH]1]

In certain embodiments, R¹ is

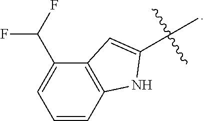 [SMILES: *c1cc2c(C(F)F)cccc2[nH]1]

In certain embodiments, R¹ is

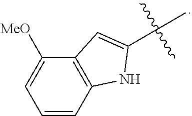 [SMILES: *c1cc2c(OC)cccc2[nH]1]

In certain embodiments, R¹ is

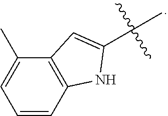 [SMILES: *c1cc2c(C)cccc2[nH]1]

In certain embodiments, R¹ is

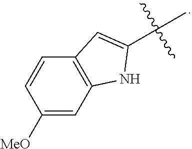 [SMILES: *c1cc2ccc(OC)cc2[nH]1]

In certain embodiments, R¹ is

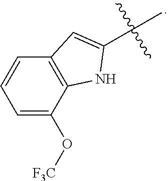 [SMILES: *c1cc2cccc(OC(F)(F)F)c2[nH]1]

In certain embodiments, R¹ is

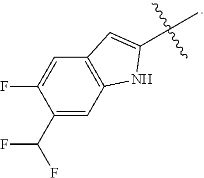 [SMILES: *c1cc2cc(F)c(C(F)F)cc2[nH]1]

In certain embodiments, R¹ is

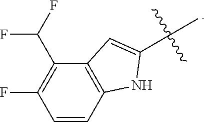 [SMILES: *c1cc2c(C(F)F)c(F)ccc2[nH]1]

In certain embodiments, R¹ is

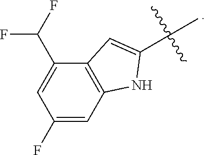 [SMILES: *c1cc2c(C(F)F)cc(F)cc2[nH]1]

In certain embodiments, R¹ is

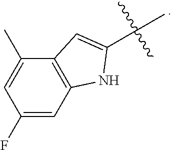 [SMILES: *c1cc2c(C)cc(F)cc2[nH]1]

In certain embodiments, R¹ is

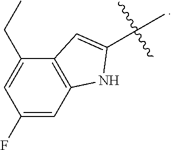 [SMILES: *c1cc2c(CC)cc(F)cc2[nH]1]

In certain embodiments, R¹ is

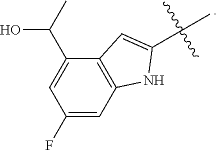 [SMILES: *c1cc2c(C(C)O)cc(F)cc2[nH]1]

In certain embodiments, R¹ is

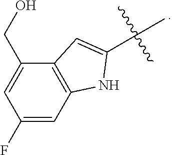 [SMILES: *c1cc2c(CO)cc(F)cc2[nH]1]

In certain embodiments, R¹ is

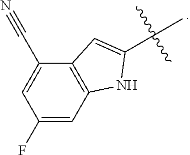 [SMILES: *c1cc2c(C#N)cc(F)cc2[nH]1]

In certain embodiments, R¹ is

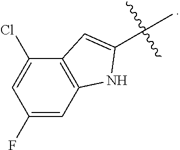 [SMILES: *c1cc2c(Cl)cc(F)cc2[nH]1]

In certain embodiments, R¹ is

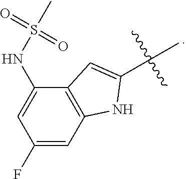 [SMILES: *c1cc2c(NS(C)(=O)=O)cc(F)cc2[nH]1]

In certain embodiments, R¹ is

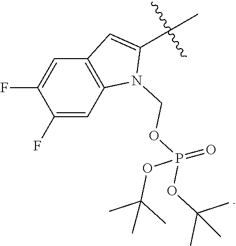 [SMILES: *c1cc2cc(F)c(F)cc2n1COP(=O)(OC(C)(C)C)OC(C)(C)C]

In certain embodiments, R¹ is

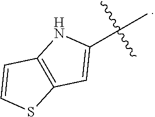 [SMILES: *c1cc2sccc2[nH]1]

In certain embodiments, R¹ is

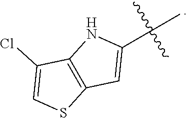 [SMILES: *c1cc2scc(Cl)c2[nH]1]

In certain embodiments, R¹ is

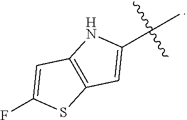 [SMILES: *c1cc2sc(F)cc2[nH]1]

In certain embodiments, R¹ is

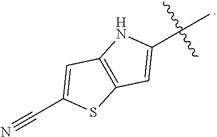 [SMILES: *c1cc2sc(C#N)cc2[nH]1]

In certain embodiments, R¹ is

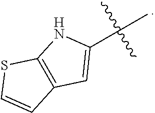 [SMILES: *c1cc2ccsc2[nH]1]

In certain embodiments, R¹ is

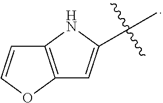 [SMILES: *c1cc2occc2[nH]1]

In certain embodiments, R¹ is

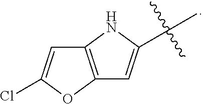 [SMILES: *c1cc2oc(Cl)cc2[nH]1]

In certain embodiments, R¹ is

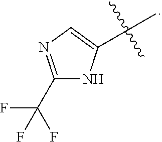 [SMILES: *c1cnc(C(F)(F)F)[nH]1]

In certain embodiments, R¹ is

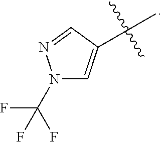 [SMILES: *c1cnn(C(F)(F)F)c1]

In certain embodiments, R¹ is

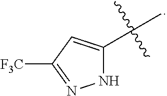 [SMILES: *c1cc(C(F)(F)F)n[nH]1]

In certain embodiments, R¹ is

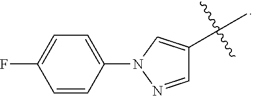 [SMILES: *c1cnn(-c2ccc(F)cc2)c1]

In certain embodiments, R¹ is

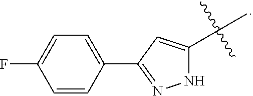 [SMILES: *c1cc(-c2ccc(F)cc2)n[nH]1]

In certain embodiments R is

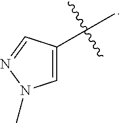 [SMILES: *c1cnn(C)c1]

In certain embodiments, R¹ is

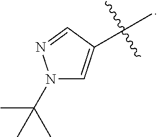 [SMILES: *c1cnn(C(C)(C)C)c1]

In certain embodiments, R¹ is

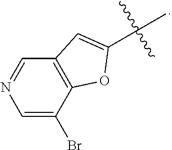 [SMILES: *c1cc2cncc(Br)c2o1]

In certain embodiments, R¹ is

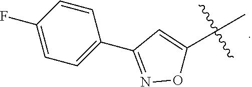 [SMILES: *c1cc(-c2ccc(F)cc2)no1]

In certain embodiments, R¹ is

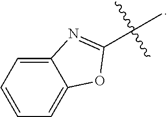 [SMILES: *c1nc2ccccc2o1]

In certain embodiments, R¹ is

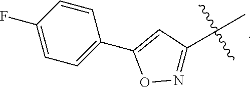 [SMILES: *c1cc(-c2ccc(F)cc2)on1]

In certain embodiments, R¹ is

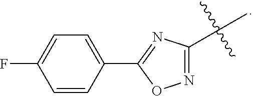 [SMILES: *c1noc(-c2ccc(F)cc2)n1]

In certain embodiments, R¹ is

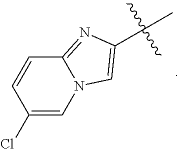 [SMILES: *c1cn2cc(Cl)ccc2n1]

In certain embodiments, R¹ is

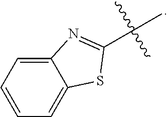 [SMILES: *c1nc2ccccc2s1]

In certain embodiments, R¹ is

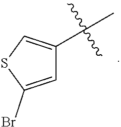 [SMILES: *c1csc(Br)c1]

In certain embodiments, R¹ is

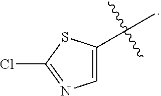 [SMILES: *c1cnc(Cl)s1]

In certain embodiments, R¹ is

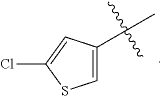 [SMILES: *c1csc(Cl)c1]

In certain embodiments, R¹ is

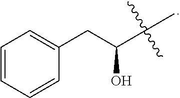 [SMILES: *[C@@H](O)Cc1ccccc1]

In certain embodiments, R¹ is

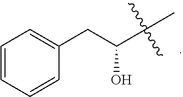 [SMILES: *[C@H](O)Cc1ccccc1]

In certain embodiments, R¹ is

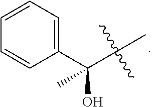 [SMILES: *[C@@](C)(O)c1ccccc1]

In certain embodiments, R¹ is

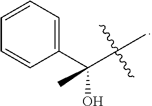 [SMILES: *[C@](C)(O)c1ccccc1]

In certain embodiments, R¹ is

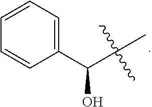 [SMILES: *[C@@H](O)c1ccccc1]

In certain embodiments, R¹ is

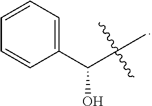 [SMILES: *[C@H](O)c1ccccc1]

In certain embodiments, R¹ is

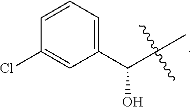 [SMILES: *[C@H](O)c1cccc(Cl)c1]

In certain embodiments, R¹ is

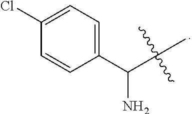 [SMILES: *C(N)c1ccc(Cl)cc1]

In certain embodiments, R¹ is

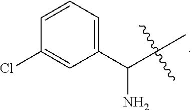 [SMILES: *C(N)c1cccc(Cl)c1]

In certain embodiments, R¹ is

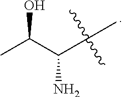 [SMILES: *[C@H](N)[C@@H](C)O]

In certain embodiments, R¹ is

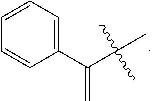 [SMILES: *C(=C)c1ccccc1]

In certain embodiments, R¹ is

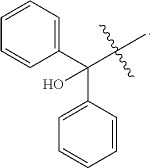 [SMILES: *C(O)(c1ccccc1)c1ccccc1]

In certain embodiments, R¹ is

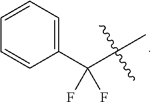 [SMILES: *C(F)(F)c1ccccc1]

In certain embodiments, R¹ is

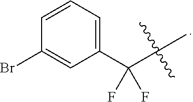 [SMILES: *C(F)(F)c1cccc(Br)c1]

In certain embodiments, R¹ is

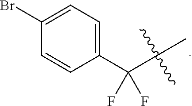 [SMILES: *C(F)(F)c1ccc(Br)cc1]

In certain embodiments, R¹ is

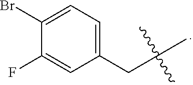 [SMILES: *Cc1ccc(Br)c(F)c1]

In certain embodiments, R¹ is

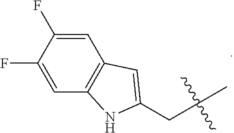 [SMILES: *Cc1cc2cc(F)c(F)cc2[nH]1]

In certain embodiments, R¹ is

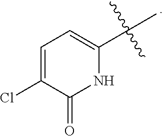 [SMILES: *c1ccc(Cl)c(=O)[nH]1]

In certain embodiments, R¹ is

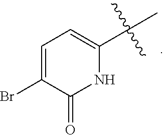 [SMILES: *c1ccc(Br)c(=O)[nH]1]

In certain embodiments, R¹ is

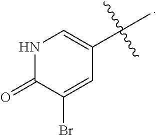 [SMILES: *c1c[nH]c(=O)c(Br)c1]

In certain embodiments, R¹ is

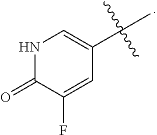 [SMILES: *c1c[nH]c(=O)c(F)c1]

In certain embodiments, R¹ is

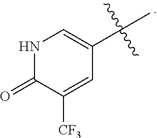 [SMILES: *c1c[nH]c(=O)c(C(F)(F)F)c1]

In certain embodiments, R¹ is

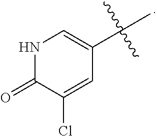 [SMILES: *c1c[nH]c(=O)c(Cl)c1]

In certain embodiments, R¹ is

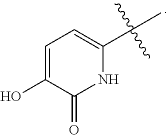 [SMILES: *c1ccc(O)c(=O)[nH]1]

In certain embodiments, R¹ is

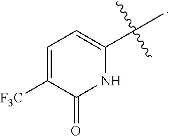 [SMILES: *c1ccc(C(F)(F)F)c(=O)[nH]1]

In certain embodiments, R¹ is

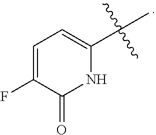 [SMILES: *c1ccc(F)c(=O)[nH]1]

In certain embodiments, R¹ is

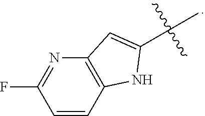 [SMILES: *c1cc2nc(F)ccc2[nH]1]

In certain embodiments, R¹ is

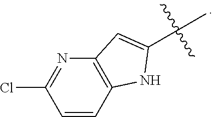 [SMILES: *c1cc2nc(Cl)ccc2[nH]1]

In certain embodiments, R¹ is

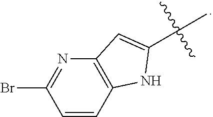 [SMILES: *c1cc2nc(Br)ccc2[nH]1]

In certain embodiments, R¹ is

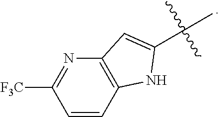 [SMILES: *c1cc2nc(C(F)(F)F)ccc2[nH]1]

In certain embodiments, R¹ is

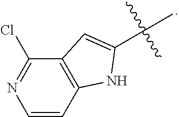 [SMILES: *c1cc2c(Cl)nccc2[nH]1]

In certain embodiments, R¹ is

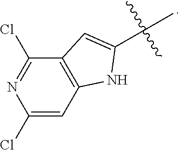 [SMILES: *c1cc2c(Cl)nc(Cl)cc2[nH]1]

In certain embodiments, R¹ is

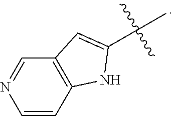 [SMILES: *c1cc2cnccc2[nH]1]

In certain embodiments, R¹ is

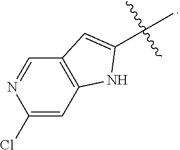 [SMILES: *c1cc2cnc(Cl)cc2[nH]1]

In certain embodiments, R¹ is

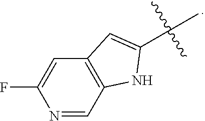 [SMILES: *c1cc2cc(F)ncc2[nH]1]

In certain embodiments, R¹ is

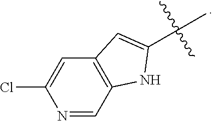 [SMILES: *c1cc2cc(Cl)ncc2[nH]1]

In certain embodiments, R¹ is

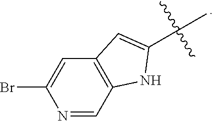 [SMILES: *c1cc2cc(Br)ncc2[nH]1]

In certain embodiments, R¹ is

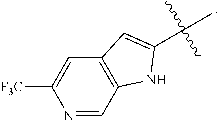 [SMILES: *c1cc2cc(C(F)(F)F)ncc2[nH]1]

In certain embodiments, R¹ is

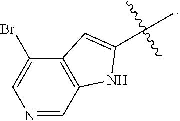 [SMILES: *c1cc2c(Br)cncc2[nH]1]

In certain embodiments, R¹ is

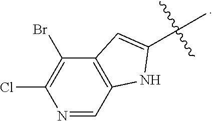 [SMILES: *c1cc2c(Br)c(Cl)ncc2[nH]1]

In certain embodiments, R¹ is

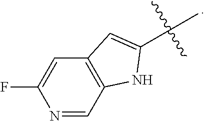 [SMILES: *c1cc2cc(F)ncc2[nH]1]

In certain embodiments, R¹ is

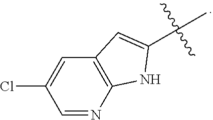 [SMILES: *c1cc2cc(Cl)cnc2[nH]1]

In certain embodiments, R¹ is

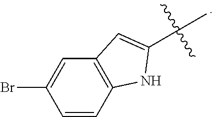 [SMILES: *c1cc2cc(Br)ccc2[nH]1]

In certain embodiments, R¹ is

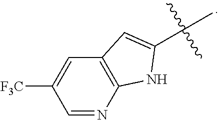 [SMILES: *c1cc2cc(C(F)(F)F)cnc2[nH]1]

In certain embodiments, R¹ is

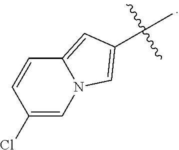 [SMILES: *c1cc2ccc(Cl)cn2c1]

In certain embodiments, R¹ is

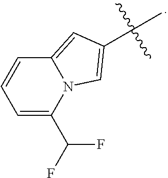 [SMILES: *c1cc2cccc(C(F)F)n2c1]

In certain embodiments, R¹ is

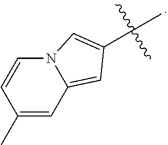 [SMILES: *c1cc2cc(C)ccn2c1]

In certain embodiments, R¹ is

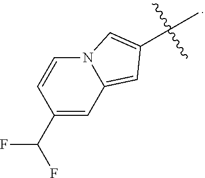 [SMILES: *c1cc2cc(C(F)F)ccn2c1]

In certain embodiments, R¹ is

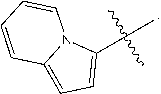 [SMILES: *c1ccc2ccccn12]

In certain embodiments, R¹ is

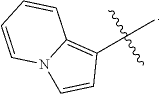 [SMILES: *c1ccn2ccccc12]

In certain embodiments, R¹ is

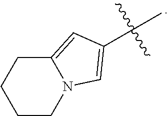 [SMILES: *c1cc2n(c1)CCCC2]

In certain embodiments, R¹ is

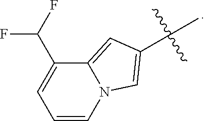 [SMILES: *c1cc2c(C(F)F)cccn2c1]

In certain embodiments, R¹ is

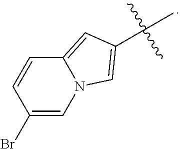 [SMILES: *c1cc2ccc(Br)cn2c1]

In certain embodiments, R¹ is

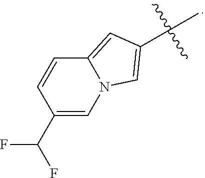 [SMILES: *c1cc2ccc(C(F)F)cn2c1]

In certain embodiments, R¹ is

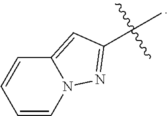 [SMILES: *c1cc2ccccn2n1]

In certain embodiments, R¹ is

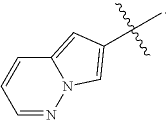 [SMILES: *c1cc2cccnn2c1]

In certain embodiments, R¹ is

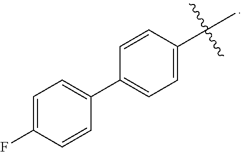 [SMILES: *c1ccc(-c2ccc(F)cc2)cc1]

In certain embodiments, R¹ is

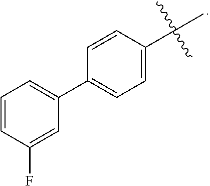 [SMILES: *c1ccc(-c2cccc(F)c2)cc1]

In certain embodiments, R¹ is

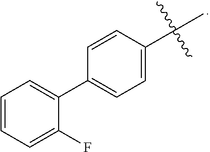 [SMILES: *c1ccc(-c2ccccc2F)cc1]

In certain embodiments, R¹ is

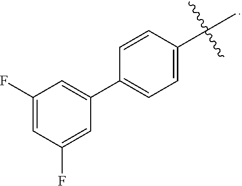 [SMILES: *c1ccc(-c2cc(F)cc(F)c2)cc1]

In certain embodiments, R¹ is

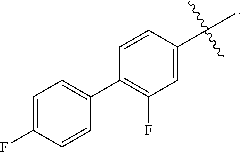 [SMILES: *c1ccc(-c2ccc(F)cc2)c(F)c1]

In certain embodiments, R¹ is

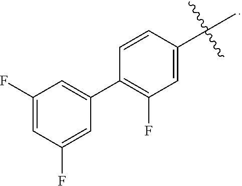 [SMILES: *c1ccc(-c2cc(F)cc(F)c2)c(F)c1]

In certain embodiments, R¹ is

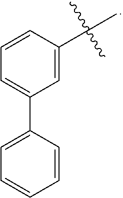 [SMILES: *c1cccc(-c2ccccc2)c1]

In certain embodiments, R¹ is

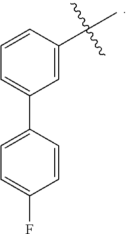 [SMILES: *c1cccc(-c2ccc(F)cc2)c1]

In certain embodiments, R¹ is

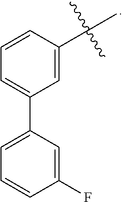 [SMILES: *c1cccc(-c2cccc(F)c2)c1]

In certain embodiments, R¹ is

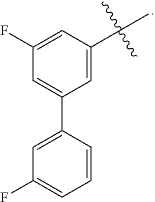 [SMILES: *c1cc(F)cc(-c2cccc(F)c2)c1]

In certain embodiments, R¹ is

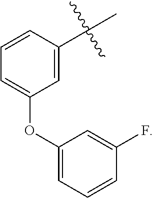 [SMILES: *c1cccc(Oc2cccc(F)c2)c1]

In certain embodiments, R¹ is

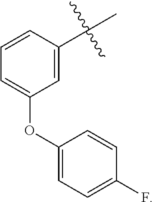 [SMILES: *c1cccc(Oc2ccc(F)cc2)c1]

In certain embodiments, R¹ is

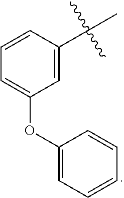 [SMILES: *c1cccc(Oc2ccccc2)c1]

In certain embodiments, R¹ is

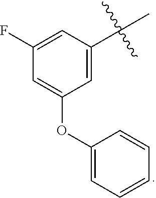 [SMILES: *c1cc(F)cc(Oc2ccccc2)c1]

In certain embodiments, R¹ is

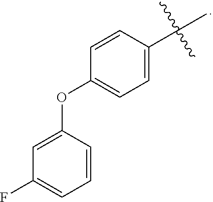 [SMILES: *c1ccc(Oc2cccc(F)c2)cc1]

In certain embodiments, R¹ is

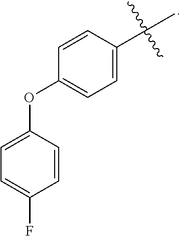 [SMILES: *c1ccc(Oc2ccc(F)cc2)cc1]

In certain embodiments, R¹ is

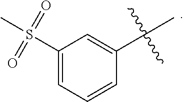 [SMILES: *c1cccc(S(C)(=O)=O)c1]

In certain embodiments, R¹ is

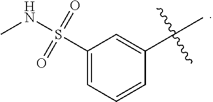 [SMILES: *c1cccc(S(=O)(=O)NC)c1]

In certain embodiments, R¹ is

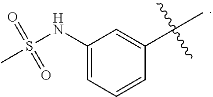 [SMILES: *c1cccc(NS(C)(=O)=O)c1]

In certain embodiments, R¹ is

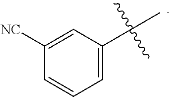 [SMILES: *c1cccc(C#N)c1]

In certain embodiments, R¹ is

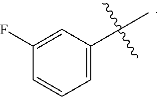 [SMILES: *c1cccc(F)c1]

In certain embodiments, R¹ is

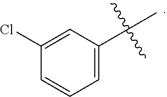 [SMILES: *c1cccc(Cl)c1]

In certain embodiments, R¹ is

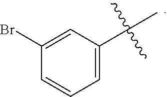 [SMILES: *c1cccc(Br)c1]

In certain embodiments, R¹ is

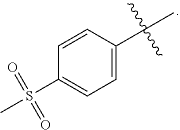 [SMILES: *c1ccc(S(C)(=O)=O)cc1]

In certain embodiments, R¹ is

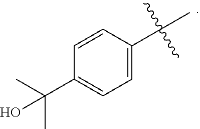 [SMILES: *c1ccc(C(C)(C)O)cc1]

In certain embodiments, R¹ is

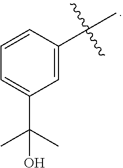 [SMILES: *c1cccc(C(C)(C)O)c1]

In certain embodiments, R¹ is

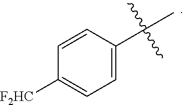 [SMILES: *c1ccc(C(F)F)cc1]

In certain embodiments, R¹ is

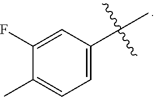 [SMILES: *c1ccc(C)c(F)c1]

In certain embodiments, R¹ is

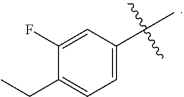 [SMILES: *c1ccc(CC)c(F)c1]

In certain embodiments, R¹ is

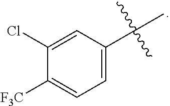 [SMILES: *c1ccc(C(F)(F)F)c(Cl)c1]

In certain embodiments, R¹ is

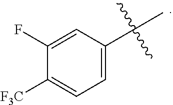 [SMILES: *c1ccc(C(F)(F)F)c(F)c1]

In certain embodiments, R¹ is

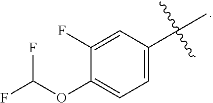 [SMILES: *c1ccc(OC(F)F)c(F)c1]

In certain embodiments, R¹ is

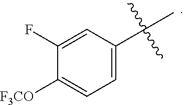 [SMILES: *c1ccc(OC(F)(F)F)c(F)c1]

In certain embodiments, R¹ is

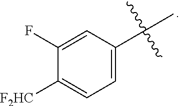 [SMILES: *c1ccc(C(F)F)c(F)c1]

In certain embodiments, R¹ is

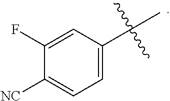 [SMILES: *c1ccc(C#N)c(F)c1]

In certain embodiments, R¹ is

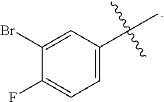 [SMILES: *c1ccc(F)c(Br)c1]

In certain embodiments, R¹ is

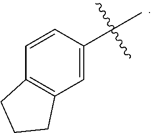 [SMILES: *c1ccc2c(c1)CCC2]

In certain embodiments, R¹ is

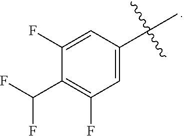 [SMILES: *c1cc(F)c(C(F)F)c(F)c1]

In certain embodiments, R¹ is

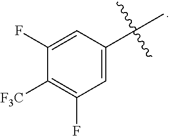 [SMILES: *c1cc(F)c(C(F)(F)F)c(F)c1]

In certain embodiments, R¹ is

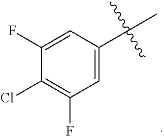 [SMILES: *c1cc(F)c(Cl)c(F)c1]

In certain embodiments, R¹ is

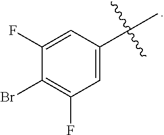 [SMILES: *c1cc(F)c(Br)c(F)c1]

In certain embodiments, R¹ is

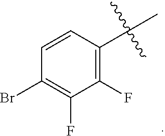 [SMILES: *c1ccc(Br)c(F)c1F]

In certain embodiments, R¹ is

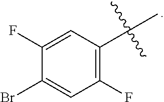 [SMILES: *c1cc(F)c(Br)cc1F]

In certain embodiments, R¹ is

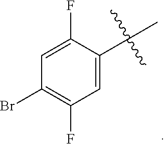 [SMILES: *c1cc(F)c(Br)cc1F]

In certain embodiments, R¹ is

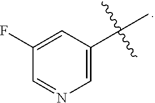 [SMILES: *c1cncc(F)c1]

In certain embodiments, R¹ is

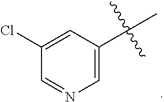 [SMILES: *c1cncc(Cl)c1]

In certain embodiments, R¹ is

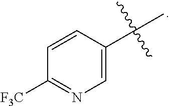 [SMILES: *c1ccc(C(F)(F)F)nc1]

In certain embodiments, R¹ is

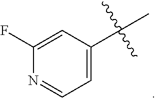 [SMILES: *c1ccnc(F)c1]

In certain embodiments, R¹ is

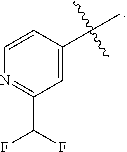 [SMILES: *c1ccnc(C(F)F)c1]

In certain embodiments, R¹ is

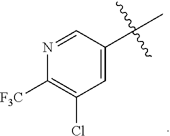 [SMILES: *c1cnc(C(F)(F)F)c(Cl)c1]

In certain embodiments, R¹ is

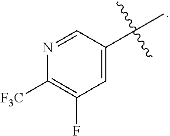 [SMILES: *c1cnc(C(F)(F)F)c(F)c1]

In certain embodiments, R¹ is

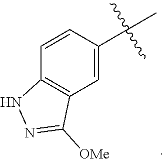 [SMILES: *c1ccc2[nH]nc(OC)c2c1]

In certain embodiments, R¹ is

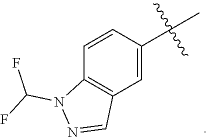 [SMILES: *c1ccc2c(cnn2C(F)F)c1]

In certain embodiments, R¹ is

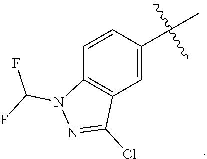 [SMILES: *c1ccc2c(c1)c(Cl)nn2C(F)F]

In certain embodiments, R¹ is

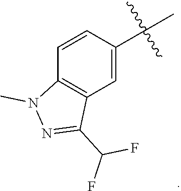 [SMILES: *c1ccc2c(c1)c(C(F)F)nn2C]

In certain embodiments, R¹ is

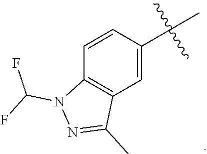 [SMILES: *c1ccc2c(c1)c(C)nn2C(F)F]

In certain embodiments, R¹ is

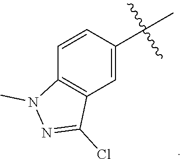 [SMILES: *c1ccc2c(c1)c(Cl)nn2C]

In certain embodiments, R¹ is

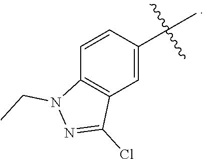 [SMILES: *c1ccc2c(c1)c(Cl)nn2CC]

In certain embodiments, R¹ is

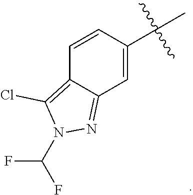 [SMILES: *c1ccc2c(Cl)n(C(F)F)nc2c1]

In certain embodiments, R¹ is

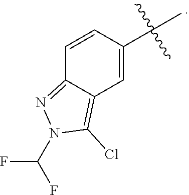 [SMILES: *c1ccc2nn(C(F)F)c(Cl)c2c1]

In certain embodiments, R¹ is

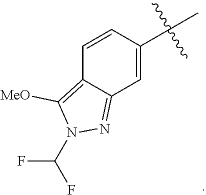 [SMILES: *c1ccc2c(OC)n(C(F)F)nc2c1]

In certain embodiments, R¹ is

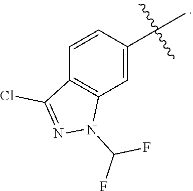 [SMILES: *c1ccc2c(Cl)nn(C(F)F)c2c1]

In certain embodiments, R¹ is

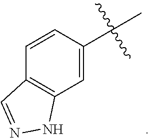 [SMILES: *c1ccc2cn[nH]c2c1]

In certain embodiments, R¹ is

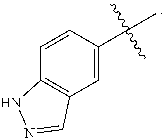 [SMILES: *c1ccc2[nH]ncc2c1]

In certain embodiments, R¹ is

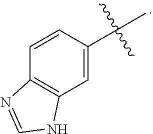 [SMILES: *c1ccc2nc[nH]c2c1]

In certain embodiments, R¹ is

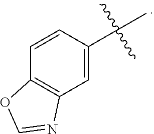 [SMILES: *c1ccc2ocnc2c1]

In certain embodiments, R¹ is

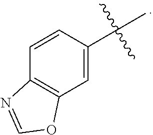 [SMILES: *c1ccc2ncoc2c1]

In certain embodiments, R¹ is

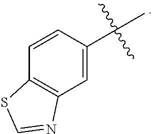 [SMILES: *c1ccc2scnc2c1]

In certain embodiments, R¹ is

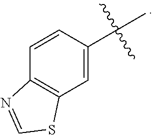 [SMILES: *c1ccc2ncsc2c1]

In certain embodiments, R¹ is

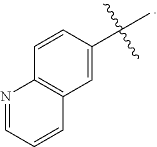 [SMILES: *c1ccc2ncccc2c1]

In certain embodiments, R¹ is

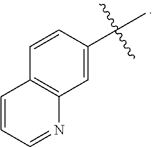 [SMILES: *c1ccc2cccnc2c1]

In certain embodiments, R¹ is

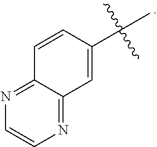 [SMILES: *c1ccc2nccnc2c1]

In certain embodiments, R¹ is

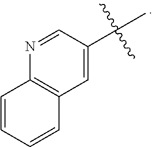 [SMILES: *c1cnc2ccccc2c1]

In certain embodiments, R¹ is

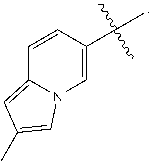 [SMILES: *c1ccc2cc(C)cn2c1]

In certain embodiments, R¹ is

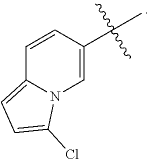 [SMILES: *c1ccc2ccc(Cl)n2c1]

In certain embodiments, R¹ is

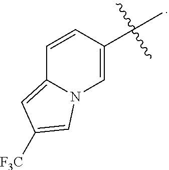 [SMILES: *c1ccc2cc(C(F)(F)F)cn2c1]

In certain embodiments, R¹ is

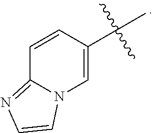 [SMILES: *c1ccc2nccn2c1]

In certain embodiments, R¹ is

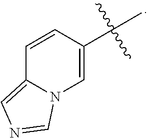 [SMILES: *c1ccc2cncn2c1]

In certain embodiments, R¹ is

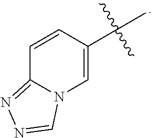 [SMILES: *c1ccc2nncn2c1]

In certain embodiments, R^{1a} is H. In certain embodiments, R^{1a} is methyl. In certain embodiments, R^{1a} is F. In certain embodiments, R^{1a} is Cl. In certain embodiments, R^{1a} is Br. In certain embodiments, R^{1a} is trifluoromethyl. In certain embodiments, R^{1a} is difluoromethyl.

In certain embodiments, R^{1b} is H. In certain embodiments, R^{1b} is methyl.

In certain embodiments, R^{1c} is H. In certain embodiments, R^{1c} is methyl.

In certain embodiments, R^{1d} is H. In certain embodiments, R^{1d} is methyl. In certain embodiments, R^{1d} is F. In certain embodiments, R^{1d} is Cl. In certain embodiments, R^{1d} is Br. In certain embodiments, R^{1d} is trifluoromethyl. In certain embodiments, R^{1d} is difluoromethyl.

In certain embodiments, R^{1e} is H. In certain embodiments, R^{1e} is methyl. In certain embodiments, R^{1e} is F. In certain embodiments, R^{1e} is Cl. In certain embodiments, R^{1e} is Br. In certain embodiments, R^{1e} is trifluoromethyl. In certain embodiments, R^{1e} is difluoromethyl.

In certain embodiments, R^{1f} is H. In certain embodiments, R^{1f} is methyl. In certain embodiments, R^{1f} is F. In certain embodiments, R^{1f} is Cl. In certain embodiments, R^{1f} is Br. In certain embodiments, R^{1f} is trifluoromethyl. In certain embodiments, R^{1f} is difluoromethyl.

In certain embodiments, R^{1g} is H. In certain embodiments, R^{1g} is methyl. In certain embodiments, R^{1g} is F. In certain embodiments, R^{1g} is Cl. In certain embodiments, R^{1g} is Br. In certain embodiments, R^{1g} is trifluoromethyl. In certain embodiments, R^{1g} is difluoromethyl.

In certain embodiments, R^{1h} is H. In certain embodiments, R^{1h} is methyl. In certain embodiments, R^{1h} is F. In certain embodiments, R^{1h} is Cl. In certain embodiments, R^{1h} is Br. In certain embodiments, R^{1h} is trifluoromethyl. In certain embodiments, R^{1h} is difluoromethyl.

In certain embodiments, R¹ⁱ is H. In certain embodiments, R¹ⁱ is methyl. In certain embodiments, R¹ⁱ is F. In certain embodiments, R¹ⁱ is Cl. In certain embodiments, R¹ⁱ is Br. In certain embodiments, R¹ⁱ is trifluoromethyl. In certain embodiments, R¹ⁱ is difluoromethyl.

In certain embodiments, R^{1j} is H. In certain embodiments, R^{1j} is methyl. In certain embodiments, R^{1j} is F. In certain embodiments, R^{1j} is Cl. In certain embodiments, R^{1j} is Br. In certain embodiments, R^{1j} is trifluoromethyl. In certain embodiments, R^{1j} is difluoromethyl.

In certain embodiments, R^{1k} is H. In certain embodiments, R^{1k} is methyl. In certain embodiments, R^{1k} is F. In certain embodiments, R^{1k} is Cl. In certain embodiments, R^{1k} is Br. In certain embodiments, R^{1k} is trifluoromethyl. In certain embodiments, R^{1k} is difluoromethyl.

In certain embodiments, R^{1l} is H. In certain embodiments, R^{1l} is methyl. In certain embodiments, R^{1l} is F. In certain embodiments, R^{1l} is Cl. In certain embodiments, R^{1l} is Br. In certain embodiments, R^{1l} is trifluoromethyl. In certain embodiments, R^{1l} is difluoromethyl.

In certain embodiments, each occurrence of R² is H. In certain embodiments, one occurrence of R² is H and the other occurrence of R² is methyl. In certain embodiments, each occurrence of R² is methyl. In certain embodiments, the two R² groups combine to form ═O.

In certain embodiments, the two R² groups combine to form ═O and R¹ is —NH(optionally substituted C₃-C₈ cycloalkyl) or —NH(optionally substituted phenyl). In certain embodiments, the two R² groups combine to form ═O and R¹ is —NH(cyclopropyl). In certain embodiments, the two R² groups combine to form ═O and R¹ is —NH(4-fluoro-3-chlorophenyl).

In certain embodiments, R³ is selected from the group consisting of H, methyl, ethyl, isopropyl, n-propyl, cyclopropyl, n-butyl, isobutyl, sec-butyl, tert-butyl, cyclobutyl, isopropylmethyl, —(CH₂)₂₋₆OH, —(CH₂)₂₋₆O(C₁-C₆ alkyl), optionally substituted benzyl, and optionally substituted phenyl.

In certain embodiments, R³ is H. In other embodiments, R³ is methyl. In yet other embodiments, R³ is ethyl. In yet other embodiments, R³ is 1-(2,2-difluoroethyl). In yet other embodiments, R³ is 1-(2,2,2-trifluoroethyl). In yet other embodiments, R³ is isopropyl. In yet other embodiments, R³ is cyclopropyl. In yet other embodiments, R³ is 1-propyl. In yet other embodiments, R³ is cyclopropylmethyl. In yet other embodiments, R³ is 1-butyl. In yet other embodiments, R³ is isobutyl. In yet other embodiments, R³ is —CH₂CH₂CH₂OH.

In certain embodiments, R^{4b} is selected from the group consisting of H and methyl. In other embodiments, R^{4b} is H. In other embodiments, R^{4b} is methyl.

In certain embodiments, R⁵ is selected from:

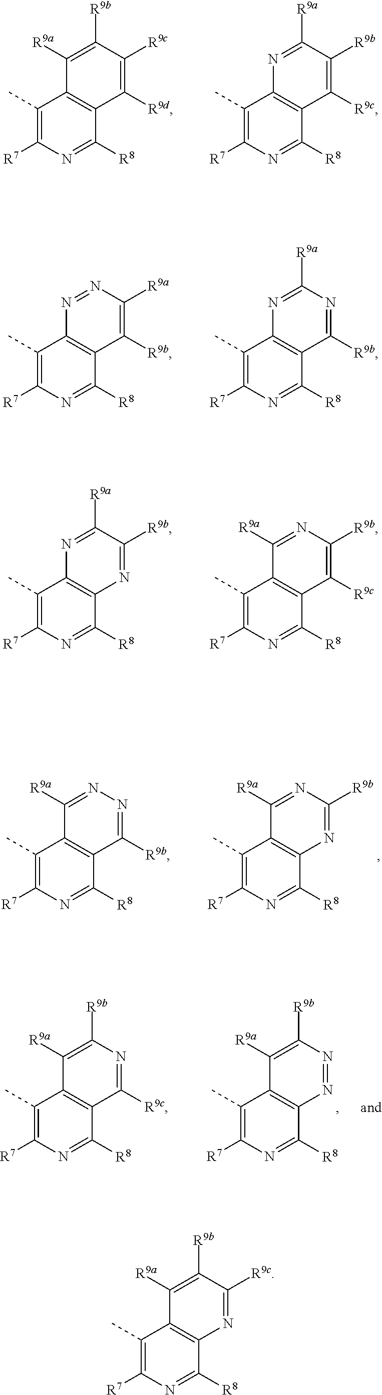

In certain embodiments, R⁵ is selected from:

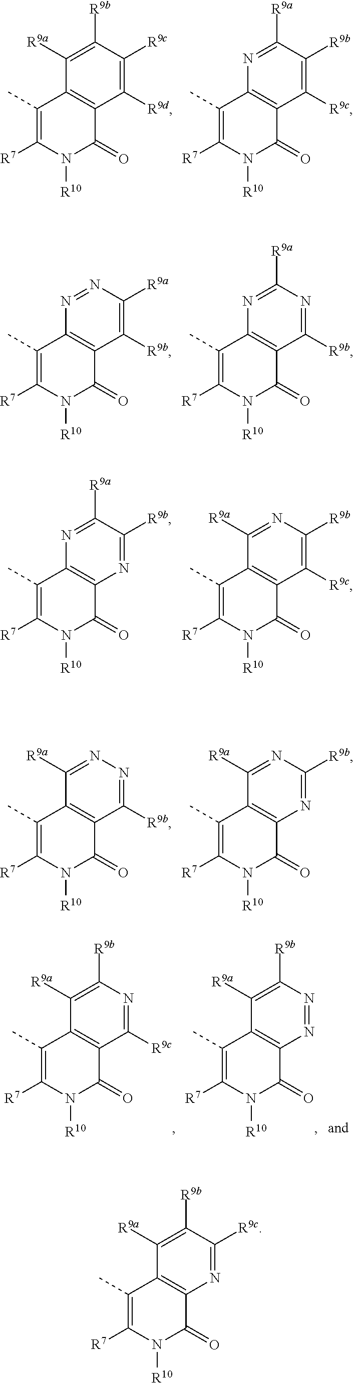

In certain embodiments, R⁵ is

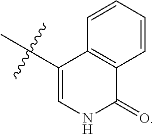 [SMILES: *c1c[nH]c(=O)c2ccccc12]

In certain embodiments, R⁵ is

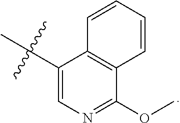 [SMILES: *c1cnc(OC)c2ccccc12]

In certain embodiments, R⁵ is

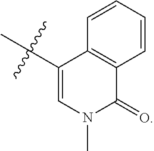 [SMILES: *c1cn(C)c(=O)c2ccccc12]

In certain embodiments, R⁵ is

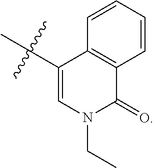 [SMILES: *c1cn(CC)c(=O)c2ccccc12]

In certain embodiments, R⁵ is

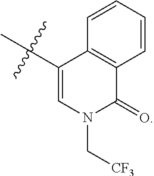 [SMILES: *c1cn(CC(F)(F)F)c(=O)c2ccccc12]

In certain embodiments, R⁵ is

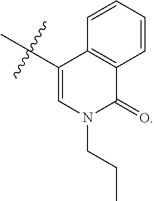 [SMILES: *c1cn(CCC)c(=O)c2ccccc12]

In certain embodiments, R⁵ is

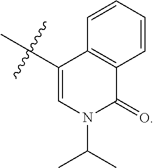 [SMILES: *c1cn(C(C)C)c(=O)c2ccccc12]

In certain embodiments, R⁵ is

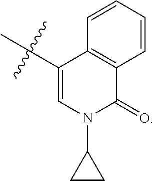 [SMILES: *c1cn(C2CC2)c(=O)c2ccccc12]

In certain embodiments, R⁵ is

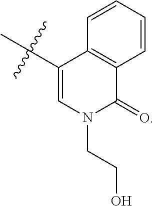 [SMILES: *c1cn(CCO)c(=O)c2ccccc12]

In certain embodiments, R⁵ is

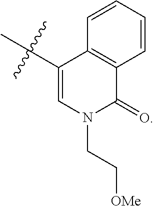 [SMILES: *c1cn(CCOC)c(=O)c2ccccc12]

In certain embodiments, R⁵ is

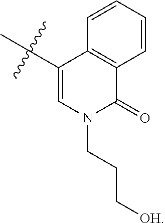 [SMILES: *c1cn(CCCO)c(=O)c2ccccc12]

In certain embodiments, R⁵ is

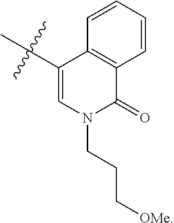 [SMILES: *c1cn(CCCOC)c(=O)c2ccccc12]

In certain embodiments, R⁵ is

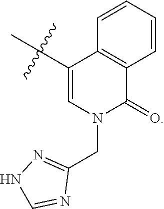 [SMILES: *c1cn(Cc2nc[nH]n2)c(=O)c2ccccc12]

In certain embodiments, R⁵ is

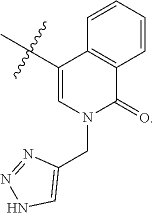 [SMILES: *c1cn(Cc2c[nH]nn2)c(=O)c2ccccc12]

In certain embodiments, R⁵ is

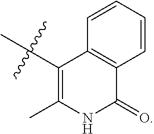 [SMILES: *c1c(C)[nH]c(=O)c2ccccc12]

In certain embodiments, R⁵ is

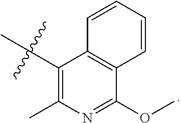

In certain embodiments, R⁵ is

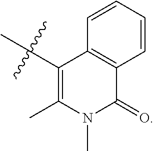 [SMILES: *c1c(C)n(C)c(=O)c2ccccc12]

In certain embodiments, R⁵ is

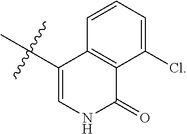 [SMILES: *c1c[nH]c(=O)c2c(Cl)cccc12]

In certain embodiments, R⁵ is. In certain embodiments, R⁵ is

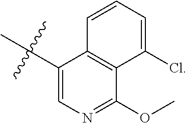 [SMILES: *c1cnc(OC)c2c(Cl)cccc12]

In certain embodiments, R⁵ is

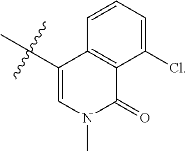 [SMILES: *c1cn(C)c(=O)c2c(Cl)cccc12]

In certain embodiments, R⁵ is

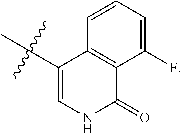 [SMILES: *c1c[nH]c(=O)c2c(F)cccc12]

In certain embodiments, R⁵ is

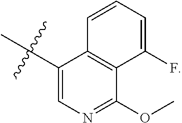 [SMILES: *c1cnc(OC)c2c(F)cccc12]

In certain embodiments, R⁵ is

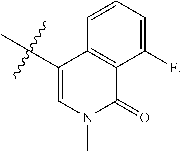 [SMILES: *c1cn(C)c(=O)c2c(F)cccc12]

In certain embodiments, R⁵ is

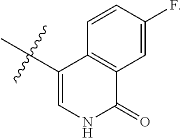 [SMILES: *c1c[nH]c(=O)c2cc(F)ccc12]

In certain embodiments, R⁵ is

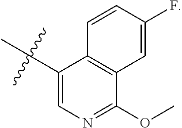 [SMILES: *c1cnc(OC)c2cc(F)ccc12]

In certain embodiments, R⁵ is

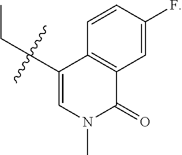

In certain embodiments, R⁵ is

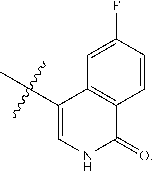 [SMILES: *c1c[nH]c(=O)c2ccc(F)cc12]

In certain embodiments, R⁵ is

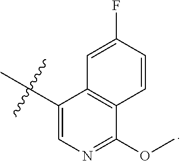 [SMILES: *c1cnc(OC)c2ccc(F)cc12]

In certain embodiments, R⁵ is

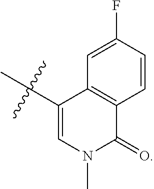 [SMILES: *c1cn(C)c(=O)c2ccc(F)cc12]

In certain embodiments, R⁵ is

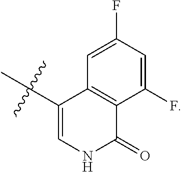 [SMILES: *c1c[nH]c(=O)c2c(F)cc(F)cc12]

In certain embodiments, R⁵ is

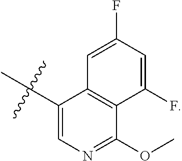 [SMILES: *c1cnc(OC)c2c(F)cc(F)cc12]

In certain embodiments, R⁵ is

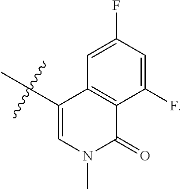 [SMILES: *c1cn(C)c(=O)c2c(F)cc(F)cc12]

In certain embodiments, R⁵ is

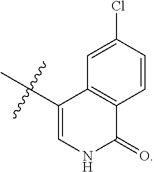 [SMILES: *c1c[nH]c(=O)c2ccc(Cl)cc12]

In certain embodiments, R⁵ is

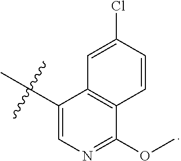 [SMILES: *c1cnc(OC)c2ccc(Cl)cc12]

In certain embodiments, R⁵ is

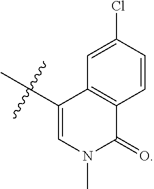 [SMILES: *c1cn(C)c(=O)c2ccc(Cl)cc12]

In certain embodiments, R⁵ is

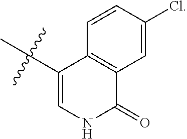 [SMILES: *c1c[nH]c(=O)c2cc(Cl)ccc12]

In certain embodiments, R⁵ is

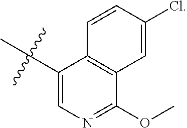 [SMILES: *c1cnc(OC)c2cc(Cl)ccc12]

In certain embodiments, R⁵ is

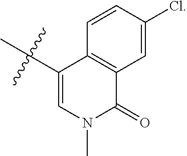 [SMILES: *c1cn(C)c(=O)c2cc(Cl)ccc12]

In certain embodiments, R⁵ is

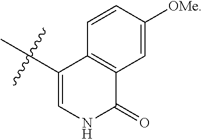 [SMILES: *c1c[nH]c(=O)c2cc(OC)ccc12]

In certain embodiments, R⁵ is

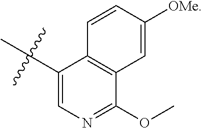 [SMILES: *c1cnc(OC)c2cc(OC)ccc12]

In certain embodiments, R⁵ is

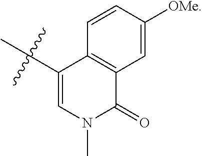 [SMILES: *c1cn(C)c(=O)c2cc(OC)ccc12]

In certain embodiments, R⁵ is

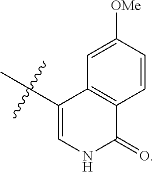 [SMILES: *c1c[nH]c(=O)c2ccc(OC)cc12]

In certain embodiments, R⁵ is

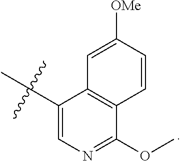 [SMILES: *c1cnc(OC)c2ccc(OC)cc12]

In certain embodiments, R⁵ is

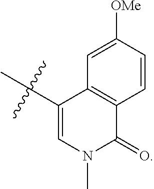 [SMILES: *c1cn(C)c(=O)c2ccc(OC)cc12]

In certain embodiments, R⁵ is

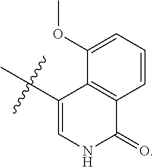 [SMILES: *c1c[nH]c(=O)c2cccc(OC)c12]

In certain embodiments, R⁵ is

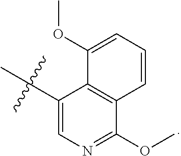 [SMILES: *c1cnc(OC)c2cccc(OC)c12]

In certain embodiments, R⁵ is

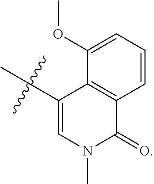 [SMILES: *c1cn(C)c(=O)c2cccc(OC)c12]

In certain embodiments, R⁵ is

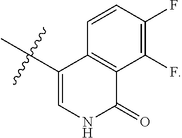 [SMILES: *c1c[nH]c(=O)c2c(F)c(F)ccc12]

In certain embodiments, R⁵ is

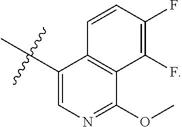 [SMILES: *c1cnc(OC)c2c(F)c(F)ccc12]

In certain embodiments, R⁵ is

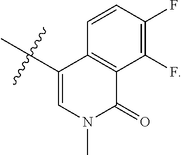 [SMILES: *c1cn(C)c(=O)c2c(F)c(F)ccc12]

In certain embodiments, R⁵ is

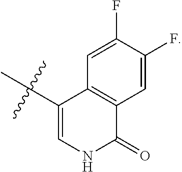 [SMILES: *c1c[nH]c(=O)c2cc(F)c(F)cc12]

In certain embodiments, R⁵ is

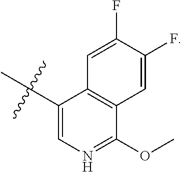 [SMILES: *c1cnc(OC)c2cc(F)c(F)cc12]

In certain embodiments, R⁵ is

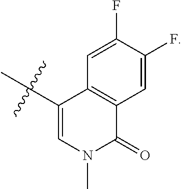 [SMILES: *c1cn(C)c(=O)c2cc(F)c(F)cc12]

In certain embodiments, R⁵ is

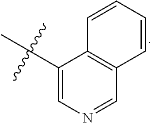 [SMILES: *c1cncc2ccccc12]

In certain embodiments, R⁵ is

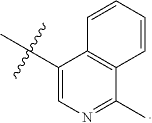 [SMILES: *c1cnc(C)c2ccccc12]

In certain embodiments, R⁵ is

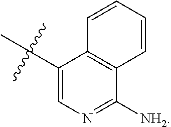 [SMILES: *c1cnc(N)c2ccccc12]

In certain embodiments, R⁵ is

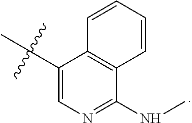 [SMILES: *c1cnc(NC)c2ccccc12]

NH In certain embodiments, R⁵ is

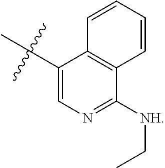 [SMILES: *c1cnc(NCC)c2ccccc12]

In certain embodiments, R⁵ is

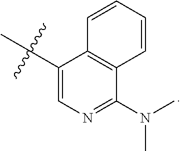 [SMILES: *c1cnc(N(C)C)c2ccccc12]

In certain embodiments, R⁵ is

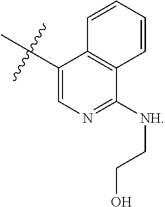 [SMILES: *c1cnc(NCCO)c2ccccc12]

In certain embodiments, R⁵ is

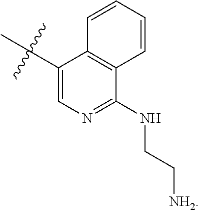 [SMILES: *c1cnc(NCCN)c2ccccc12]

In certain embodiments, R⁵ is

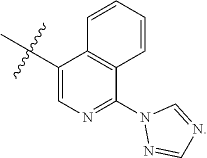 [SMILES: *c1cnc(-n2cncn2)c2ccccc12]

In certain embodiments, R⁵ is

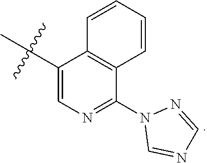 [SMILES: *c1cnc(-n2cncn2)c2ccccc12]

In certain embodiments, R⁵ is

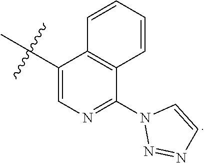 [SMILES: *c1cnc(-n2ccnn2)c2ccccc12]

In certain embodiments, R⁵ is

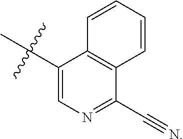 [SMILES: *c1cnc(C#N)c2ccccc12]

In certain embodiments, R⁵ is

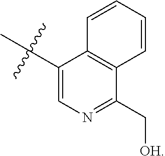 [SMILES: *c1cnc(CO)c2ccccc12]

In certain embodiments, R⁵ is

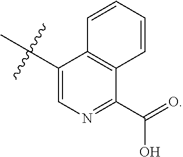 [SMILES: *c1cnc(C(=O)O)c2ccccc12]

In certain embodiments, R⁵ is

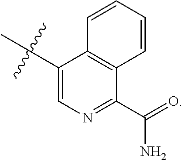 [SMILES: *c1cnc(C(N)=O)c2ccccc12]

In certain embodiments, R⁵ is

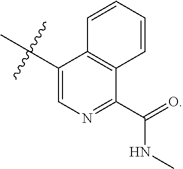 [SMILES: *c1cnc(C(=O)NC)c2ccccc12]

In certain embodiments, R⁵ is

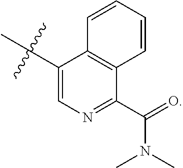 [SMILES: *c1cnc(C(=O)N(C)C)c2ccccc12]

In certain embodiments, R⁵ is

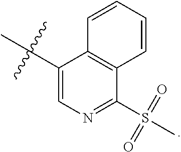 [SMILES: *c1cnc(S(C)(=O)=O)c2ccccc12]

In certain embodiments, R⁵ is

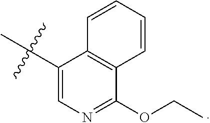 [SMILES: *c1cnc(OCC)c2ccccc12]

In certain embodiments, R⁵ is

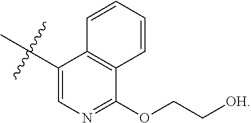 [SMILES: *c1cnc(OCCO)c2ccccc12]

In certain embodiments, R⁵ is

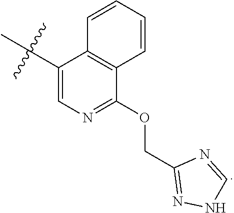 [SMILES: *c1cnc(OCc2nc[nH]n2)c2ccccc12]

In certain embodiments, R⁵ is

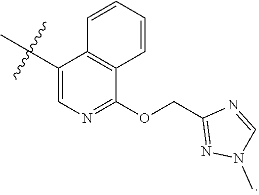 [SMILES: *c1cnc(OCc2ncn(C)n2)c2ccccc12]

In certain embodiments, R⁵ is

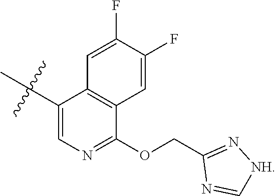 [SMILES: *c1cnc(OCc2nc[nH]n2)c2cc(F)c(F)cc12]

In certain embodiments, R⁵ is

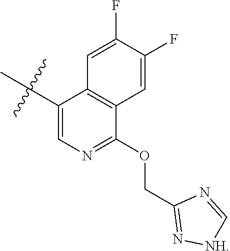 [SMILES: *c1cnc(OCc2nc[nH]n2)c2cc(F)c(F)cc12]

In certain embodiments, R⁵ is

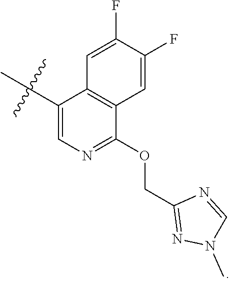 [SMILES: *c1cnc(OCc2ncn(C)n2)c2cc(F)c(F)cc12]

In certain embodiments, R⁵ is

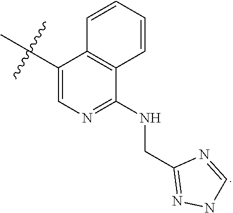 [SMILES: *c1cnc(NCc2nc[nH]n2)c2ccccc12]

In certain embodiments, R⁵ is

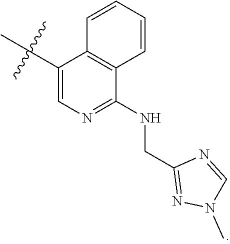 [SMILES: *c1cnc(NCc2ncn(C)n2)c2ccccc12]

In certain embodiments, R⁵ is

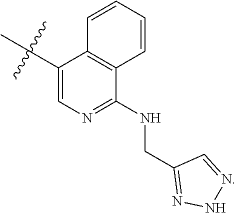 [SMILES: *c1cnc(NCc2cn[nH]n2)c2ccccc12]

In certain embodiments, R⁵ is

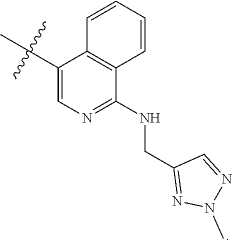 [SMILES: *c1cnc(NCc2cnn(C)n2)c2ccccc12]

In certain embodiments, R⁵ is

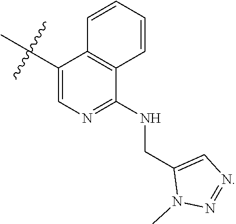 [SMILES: *c1cnc(NCc2cnnn2C)c2ccccc12]

In certain embodiments, R⁵ is

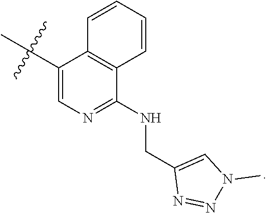 [SMILES: *c1cnc(NCc2cn(C)nn2)c2ccccc12]

In certain embodiments, R⁵ is

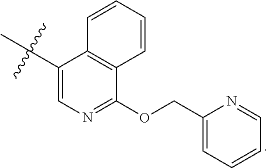 [SMILES: *c1cnc(OCc2ccccn2)c2ccccc12]

In certain embodiments, R⁵ is

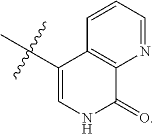 [SMILES: *c1c[nH]c(=O)c2ncccc12]

In certain embodiments, R⁵ is

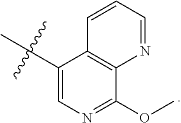 [SMILES: *c1cnc(OC)c2ncccc12]

In certain embodiments, R⁵ is

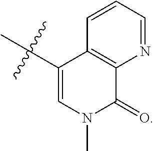 [SMILES: *c1cn(C)c(=O)c2ncccc12]

In certain embodiments, R⁵ is

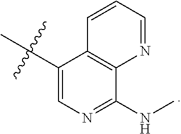 [SMILES: *c1cnc(NC)c2ncccc12]

In certain embodiments, R⁵ is

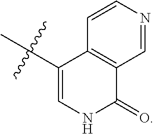 [SMILES: *c1c[nH]c(=O)c2cnccc12]

In certain embodiments, R⁵ is

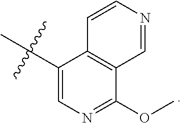 [SMILES: *c1cnc(OC)c2cnccc12]

In certain embodiments, R⁵ is

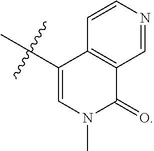 [SMILES: *c1cn(C)c(=O)c2cnccc12]

In certain embodiments, R⁵ is

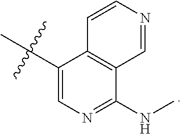 [SMILES: *c1cnc(NC)c2cnccc12]

In certain embodiments, R⁵ is

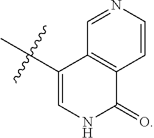 [SMILES: *c1c[nH]c(=O)c2ccncc12]

In certain embodiments, R⁵ is

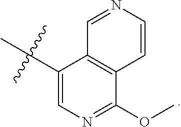 [SMILES: *c1cnc(OC)c2ccncc12]

In certain embodiments, R⁵ is

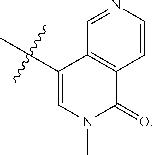 [SMILES: *c1cn(C)c(=O)c2ccncc12]

In certain embodiments, R⁵ is

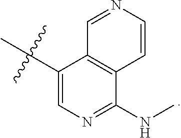 [SMILES: *c1cnc(NC)c2ccncc12]

In certain embodiments, R⁵ is

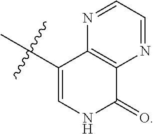 [SMILES: *c1c[nH]c(=O)c2nccnc12]

In certain embodiments, R⁵ is

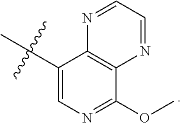 [SMILES: *c1cnc(OC)c2nccnc12]

In certain embodiments, R⁵ is

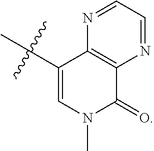 [SMILES: *c1cn(C)c(=O)c2nccnc12]

In certain embodiments, R⁵ is

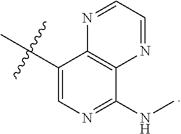 [SMILES: *c1cnc(NC)c2nccnc12]

In certain embodiments, R⁷ is H.

In certain embodiments, R⁸ is OMe.

In certain embodiments, R^{9a} is F. In certain embodiments, R^{9a} is H. In certain embodiments, R^{9b} is F. In certain embodiments, R^{9b} is H. In certain embodiments, R^{9c} is F. In certain embodiments, R^{9c} is H. In certain embodiments, R^{9d} is F. In certain embodiments, R^{9d} is H.

In certain embodiments, R¹⁰ is H. In certain embodiments, R¹⁰ is methyl.

In certain embodiments, the compound of the disclosure is any compound disclosed herein, or a salt, solvate, prodrug, isotopically labeled, stereoisomer, any mixture of stereoisomers, tautomer, and/or any mixture of tautomers thereof.

In certain embodiments, the compound is at least one selected from Table 1, or a salt, solvate, prodrug, isotopically labeled, stereoisomer, any mixture of stereoisomers, tautomer, and/or any mixture of tautomers thereof. In certain embodiments, the compound is at least one of.
N-(1-(6,7-difluoro-1-oxo-1,2-dihydroisoquinolin-4-yl)ethyl)-N-methyl-1H-indole-2-carboxamide;
N-(1-(6,7-difluoro-1-oxo-1,2-dihydroisoquinolin-4-yl)ethyl)-N-isobutyl-1H-indole-2-carboxamide;
N-(1-(6,7-difluoro-1-oxo-1,2-dihydroisoquinolin-4-yl)ethyl)-N-ethyl-1H-indole-2-carboxamide;
N-(1-(6,7-difluoro-1-oxo-1,2-dihydroisoquinolin-4-yl)ethyl)-4-fluoro-N-methyl-1H-indole-2-carboxamide;
N-(1-(6,7-difluoro-1-oxo-1,2-dihydroisoquinolin-4-yl)ethyl)-5-fluoro-N-methyl-1H-indole-2-carboxamide;
N-(1-(6,7-difluoro-1-oxo-1,2-dihydroisoquinolin-4-yl)ethyl)-6-fluoro-N-methyl-1H-indole-2-carboxamide;
N-(1-(6,7-difluoro-1-oxo-1,2-dihydroisoquinolin-4-yl)ethyl)-7-fluoro-N-methyl-1H-indole-2-carboxamide;
N-(1-(6,7-difluoro-1-oxo-1,2-dihydroisoquinolin-4-yl)ethyl)-5,6-difluoro-N-methyl-1H-indole-2-carboxamide;
N-(1-(6,7-difluoro-1-oxo-1,2-dihydroisoquinolin-4-yl)ethyl)-4,6-difluoro-N-methyl-1H-indole-2-carboxamide;
N-(1-(6,7-difluoro-1-oxo-1,2-dihydroisoquinolin-4-yl)ethyl)-N-methyl-1,2,3,4-tetrahydroisoquinoline-3-carboxamide;
N-(1-(6,7-difluoro-1-oxo-1,2-dihydroisoquinolin-4-yl)ethyl)-N-methylindoline-2-carboxamide;
N-(1-(6,7-difluoro-1-oxo-1,2-dihydroisoquinolin-4-yl)ethyl)-N-methyl-1H-benzo[d]imidazole-2-carboxamide;
N-(1-(6,7-difluoro-1-oxo-1,2-dihydroisoquinolin-4-yl)ethyl)-N-methylbenzofuran-2-carboxamide;
N-(1-(6,7-difluoro-1-oxo-1,2-dihydroisoquinolin-4-yl)ethyl)-N-(3-hydroxypropyl)-1H-indole-2-carboxamide;
N-(1-(6,7-difluoro-1-oxo-1,2-dihydroisoquinolin-4-yl)ethyl)-4,5-difluoro-N-methyl-1H-indole-2-carboxamide;
N-(1-(6,7-difluoro-1-oxo-1,2-dihydroisoquinolin-4-yl)ethyl)-N-methylindoline-2-carboxamide;
N-(1-(6,7-difluoro-1-oxo-1,2-dihydroisoquinolin-4-yl)ethyl)-N-methyl-1,2,3,4-tetrahydroisoquinoline-3-carboxamide;
N-(1-(6,7-difluoro-1-oxo-1,2-dihydroisoquinolin-4-yl)ethyl)-N-methyl-4,5,6,7-tetrahydro-1H-indole-2-carboxamide;
N-(1-(6,7-difluoro-1-oxo-1,2-dihydroisoquinolin-4-yl)ethyl)-N,1-dimethyl-1H-indole-2-carboxamide;
N-(1-(6,7-difluoro-1-oxo-1,2-dihydroisoquinolin-4-yl)ethyl)-N-methylimidazo[1,2-a]pyridine-2-carboxamide;
N-(1-(6,7-difluoro-1-oxo-1,2-dihydroisoquinolin-4-yl)ethyl)-N-methyl-1,4,5,6-tetrahydrocyclopenta[b]pyrrole-2-carboxamide;
N-(1-(6,7-difluoro-1-oxo-1,2-dihydroisoquinolin-4-yl)ethyl)-5,6-difluoro-N-methyl-1H-benzo[d]imidazole-2-carboxamide;
N-(1-(6,7-difluoro-1-oxo-1,2-dihydroisoquinolin-4-yl)ethyl)-N-methyl-1H-pyrrolo[2,3-b]pyridine-2-carboxamide;
N-(1-(6,7-difluoro-1-oxo-1,2-dihydroisoquinolin-4-yl)ethyl)-N-methyl-1H-indole-3-carboxamide;
N-(1-(6,7-difluoro-1-oxo-1,2-dihydroisoquinolin-4-yl)ethyl)-N-methyl-1H-indazole-3-carboxamide;
N-(1-(7,8-difluoro-1-oxo-1,2-dihydroisoquinolin-4-yl)ethyl)-N-methyl-1,4,5,6-tetrahydrocyclopenta[b]pyrrole-2-carboxamide;
N-(1-(6,7-difluoro-1-oxo-1,2-dihydroisoquinolin-4-yl)ethyl)-2-(1H-indol-2-yl)-N-methylacetamide;
3-chloro-N-(1-(6,7-difluoro-1-oxo-1,2-dihydroisoquinolin-4-yl)ethyl)-N-methyl-1H-indole-2-carboxamide;
N-(1-(6,7-difluoro-1-oxo-1,2-dihydroisoquinolin-4-yl)ethyl)-5-fluoro-N-methylindoline-2-carboxamide;
N-(1-(7,8-difluoro-1-oxo-1,2-dihydroisoquinolin-4-yl)ethyl)-4,6-difluoro-N-methylindoline-2-carboxamide;
N-(1-(6,7-difluoro-1-oxo-1,2-dihydroisoquinolin-4-yl)ethyl)-N-methylindolizine-2-carboxamide;
N-(1-(7,8-difluoro-1-oxo-1,2-dihydroisoquinolin-4-yl)ethyl)-N-methylindolizine-2-carboxamide;
N-(1-(7,8-difluoro-1-oxo-1,2-dihydroisoquinolin-4-yl)ethyl)-5-fluoro-N-methylindoline-2-carboxamide;
8-chloro-N-(1-(6,7-difluoro-1-oxo-1,2-dihydroisoquinolin-4-yl)ethyl)-N-methylindolizine-2-carboxamide;
N-(1-(6,7-difluoro-1-oxo-1,2-dihydroisoquinolin-4-yl)ethyl)-8-fluoro-N-methylindolizine-2-carboxamide;
N-(1-(7,8-difluoro-1-oxo-1,2-dihydroisoquinolin-4-yl)ethyl)-8-fluoro-N-methylindolizine-2-carboxamide;
N-(1-(6,7-difluoro-1-oxo-1,2-dihydroisoquinolin-4-yl)ethyl)-N-(3-hydroxypropyl)-2-(1H-indol-2-yl)acetamide;
N-(1-(7,8-difluoro-1-oxo-1,2-dihydroisoquinolin-4-yl)ethyl)-N-methyl-6-oxo-1,6-dihydropyridine-2-carboxamide;
N-(1-(6,7-difluoro-1-oxo-1,2-dihydroisoquinolin-4-yl)ethyl)-5,5-difluoro-N-methyl-4,5,6,7-tetrahydro-1H-indole-2-carboxamide;
N-(1-(7,8-difluoro-1-oxo-1,2-dihydroisoquinolin-4-yl)ethyl)-5,5-difluoro-N-methyl-4,5,6,7-tetrahydro-1H-indole-2-carboxamide;
8-chloro-N-(1-(7,8-difluoro-1-oxo-1,2-dihydroisoquinolin-4-yl)ethyl)-N-methylindolizine-2-carboxamide;
3-chloro-N-(1-(6,7-difluoro-1-oxo-1,2-dihydroisoquinolin-4-yl)ethyl)-N-(3-hydroxypropyl)-1H-indole-2-carboxamide;
7-chloro-N-(1-(6,7-difluoro-1-oxo-1,2-dihydroisoquinolin-4-yl)ethyl)-N-methylindolizine-2-carboxamide;
N-(1-(6,7-difluoro-1-oxo-1,2-dihydroisoquinolin-4-yl)ethyl)-4,6-difluoro-N-methylindoline-2-carboxamide;
N-(1-(6,7-difluoro-1-oxo-1,2-dihydroisoquinolin-4-yl)ethyl)-6-fluoro-N-methylindolizine-2-carboxamide;
N-(1-(7,8-difluoro-1-oxo-1,2-dihydroisoquinolin-4-yl)ethyl)-N,3,3-trimethylindoline-2-carboxamide;
N-(1-(6,7-difluoro-1-oxo-1,2-dihydroisoquinolin-4-yl)ethyl)-7-fluoro-N-methylindolizine-2-carboxamide;
6-chloro-N-(1-(6,7-difluoro-1-oxo-1,2-dihydroisoquinolin-4-yl)ethyl)-N-methylindolizine-2-carboxamide;
N-(1-(6,7-difluoro-1-oxo-1,2-dihydroisoquinolin-4-yl)ethyl)-N,3,3-trimethylindoline-2-carboxamide;
N-(1-(6,7-Difluoro-1-oxo-1,2-dihydroisoquinolin-4-yl)ethyl)-N-isobutylindolizine-2-carboxamide;
N-(1-(6,7-difluoro-1-oxo-1,2-dihydroisoquinolin-4-yl)ethyl)-3-fluoro-N-isobutyl-4-(trifluoromethyl)benzamide;
N-(1-(6,7-Difluoro-1-oxo-1,2-dihydroisoquinolin-4-yl)ethyl)-3-fluoro-N-methyl-4-(trifluoromethyl) benzamide;
4-Chloro-N-(1-(6,7-difluoro-1-oxo-1,2-dihydroisoquinolin-4-yl)ethyl)-3-fluoro-N-methylbenzamide;
N-(1-(6,7-Difluoro-1-oxo-1,2-dihydroisoquinolin-4-yl)ethyl)-3,4,5-trifluoro-N-methylbenzamide;
N-(1-(6,7-Difluoro-1-oxo-1,2-dihydroisoquinolin-4-yl)ethyl)-3-(difluoromethyl)-N-methylbenzamide;
N-(1-(6,7-Difluoro-1-oxo-1,2-dihydroisoquinolin-4-yl)ethyl)-N-methylbenzamide; N-(1-(6,7-Difluoro-1-oxo-1,2-dihydroisoquinolin-4-yl)ethyl)-N-methyl-4-(trifluoromethyl) benzamide;
4-Chloro-N-(1-(6,7-difluoro-1-oxo-1,2-dihydroisoquinolin-4-yl)ethyl)-N-methylbenzamide;
N-(1-(6,7-Difluoro-1-oxo-1,2-dihydroisoquinolin-4-yl)ethyl)-3-(difluoromethyl)-4-fluoro-N-methylbenzamide;
3-Chloro-N-(1-(6,7-difluoro-1-oxo-1,2-dihydroisoquinolin-4-yl)ethyl)-4-fluoro-N-methylbenzamide;
N-(1-(6,7-Difluoro-1-oxo-1,2-dihydroisoquinolin-4-yl)ethyl)-3,4-difluoro-N-methylbenzamide;
N-(1-(6,7-Difluoro-1-oxo-1,2-dihydroisoquinolin-4-yl)ethyl)-N,1-dimethyl-1H-indole-5-carboxamide;
N-(1-(6,7-Difluoro-1-oxo-1,2-dihydroisoquinolin-4-yl)ethyl)-N,1-dimethyl-1H-indole-6-carboxamide;
N1-Cyclopropyl-N2-(1-(6,7-difluoro-1-oxo-1,2-dihydroisoquinolin-4-yl)ethyl)-N2-methyloxalamide;
N1-(3-Chloro-4-fluorophenyl)-N2-(1-(6,7-difluoro-1-oxo-1,2-dihydroisoquinolin-4-yl)ethyl)-N2-methyloxalamide;
N-(1-(7,8-difluoro-1-oxo-1,2-dihydroisoquinolin-4-yl)ethyl)-3-fluoro-N-isobutyl-4-(trifluoromethyl)benzamide;
3-fluoro-N-methyl-N-(1-(1-oxo-1,2-dihydroisoquinolin-4-yl)ethyl)-4-(trifluoromethyl)benzamide;
4-bromo-N-methyl-N-(1-(1-oxo-1,2-dihydroisoquinolin-4-yl)ethyl)benzamide; N-methyl-N-(1-(1-oxo-1,2-dihydroisoquinolin-4-yl)ethyl)-4-(trifluoromethyl)benzamide;
4-chloro-3-fluoro-N-methyl-N-(1-(1-oxo-1,2-dihydroisoquinolin-4-yl)ethyl)benzamide;
3-chloro-4-fluoro-N-methyl-N-(1-(1-oxo-1,2-dihydroisoquinolin-4-yl)ethyl)benzamide;
4-bromo-3-fluoro-N-methyl-N-(1-(1-oxo-1,2-dihydroisoquinolin-4-yl)ethyl)benzamide;
2-(4-chlorophenyl)-N-methyl-N-(1-(1-oxo-1,2-dihydroisoquinolin-4-yl)ethyl)acetamide; N-methyl-N-(1-(1-oxo-1,2-dihydroisoquinolin-4-yl)ethyl)indolizine-2-carboxamide;
7-fluoro-N-methyl-N-(1-(1-oxo-1,2-dihydroisoquinolin-4-yl)ethyl)indolizine-2-carboxamide;
8-chloro-N-methyl-N-(1-(1-oxo-1,2-dihydroisoquinolin-4-yl)ethyl)indolizine-2-carboxamide;
N-methyl-N-(1-(1-oxo-1,2-dihydroisoquinolin-4-yl)ethyl)-1H-indole-2-carboxamide;
4-fluoro-N-methyl-N-(1-(1-oxo-1,2-dihydroisoquinolin-4-yl)ethyl)-1H-indole-2-carboxamide;
5-fluoro-N-methyl-N-(1-(1-oxo-1,2-dihydroisoquinolin-4-yl)ethyl)-1H-indole-2-carboxamide;
4,6-difluoro-N-methyl-N-(1-(1-oxo-1,2-dihydroisoquinolin-4-yl)ethyl)-1H-indole-2-carboxamide;
5,6-difluoro-N-methyl-N-(1-(1-oxo-1,2-dihydroisoquinolin-4-yl)ethyl)-1H-indole-2-carboxamide;
N-methyl-N-((R)-1-(1-oxo-1,2-dihydroisoquinolin-4-yl)ethyl)indoline-2-carboxamide;
4-bromo-N-(1-(6,7-difluoro-2-methyl-1-oxo-1,2-dihydroisoquinolin-4-yl)ethyl)-3-fluoro-N-methylbenzamide;
4-bromo-N-(1-(6,7-difluoro-1-methoxyisoquinolin-4-yl)ethyl)-N-methylbenzamide;
N-(1-(6,7-difluoro-2-methyl-1-oxo-1,2-dihydroisoquinolin-4-yl)ethyl)-3-fluoro-N-methyl-4-(trifluoromethyl)benzamide;
4-bromo-N-(1-(6,7-difluoro-2-methyl-1-oxo-1,2-dihydroisoquinolin-4-yl)ethyl)-N-methylbenzamide;
N-(1-(6,7-difluoro-1-methoxyisoquinolin-4-yl)ethyl)-5,6-difluoro-N-methyl-1H-indole-2-carboxamide;
N-(1-(6,7-difluoro-2-methyl-1-oxo-1,2-dihydroisoquinolin-4-yl)ethyl)-8-fluoro-N-methylindolizine-2-carboxamide;
N-(1-(6,7-difluoro-2-methyl-1-oxo-1,2-dihydroisoquinolin-4-yl)ethyl)-5,6-difluoro-N-methyl-1H-indole-2-carboxamide;
N-(1-(6,7-difluoro-1-methoxyisoquinolin-4-yl)ethyl)-3-fluoro-N-methyl-4-(trifluoromethyl)benzamide;
4-bromo-N-(1-(6,7-difluoro-1-methoxyisoquinolin-4-yl)ethyl)-3-fluoro-N-methylbenzamide;
N-(1-(6,7-difluoro-1-methoxyisoquinolin-4-yl)ethyl)-8-fluoro-N-methylindolizine-2-carboxamide;
N-(1-(6,7-difluoro-1-methoxyisoquinolin-4-yl)ethyl)-N-methylindoline-2-carboxamide;
N-(1-(6,7-difluoro-2-methyl-1-oxo-1,2-dihydroisoquinolin-4-yl)ethyl)-N-methylindoline-2-carboxamide;
N-(1-(6,7-difluoro-1-oxo-1,2-dihydroisoquinolin-4-yl)ethyl)-N-methylindolizine-6-carboxamide;
2-chloro-N-(1-(6,7-difluoro-1-oxo-1,2-dihydroisoquinolin-4-yl)ethyl)-N-methyl-4H-thieno[3,2-b]pyrrole-5-carboxamide;
N-(1-(6,7-difluoro-1-oxo-1,2-dihydroisoquinolin-4-yl)ethyl)-N-methylindolizine-7-carboxamide;
   or a salt, solvate, prodrug, isotopically labeled, stereoisomer, any mixture of stereoisomers, tautomer, and/or any mixture of tautomers thereof.

In certain embodiments, the compound is at least one of:
(R)-N-(1-(6,7-difluoro-1-oxo-1,2-dihydroisoquinolin-4-yl)ethyl)-N-methyl-1H-indole-2-carboxamide;
(S)-N-(1-(6,7-difluoro-1-oxo-1,2-dihydroisoquinolin-4-yl)ethyl)-N-methyl-1H-indole-2-carboxamide;
(R)-N-(1-(6,7-difluoro-1-oxo-1,2-dihydroisoquinolin-4-yl)ethyl)-N-isobutyl-1H-indole-2-carboxamide;
(S)-N-(1-(6,7-difluoro-1-oxo-1,2-dihydroisoquinolin-4-yl)ethyl)-N-isobutyl-1H-indole-2-carboxamide;
(R)-N-(1-(6,7-difluoro-1-oxo-1,2-dihydroisoquinolin-4-yl)ethyl)-N-ethyl-1H-indole-2-carboxamide;
(S)-N-(1-(6,7-difluoro-1-oxo-1,2-dihydroisoquinolin-4-yl)ethyl)-N-ethyl-1H-indole-2-carboxamide;
(R)-N-(1-(6,7-difluoro-1-oxo-1,2-dihydroisoquinolin-4-yl)ethyl)-4-fluoro-N-methyl-1H-indole-2-carboxamide;
(S)-N-(1-(6,7-difluoro-1-oxo-1,2-dihydroisoquinolin-4-yl)ethyl)-4-fluoro-N-methyl-1H-indole-2-carboxamide;
(R)-N-(1-(6,7-difluoro-1-oxo-1,2-dihydroisoquinolin-4-yl)ethyl)-5-fluoro-N-methyl-1H-indole-2-carboxamide;
(S)-N-(1-(6,7-difluoro-1-oxo-1,2-dihydroisoquinolin-4-yl)ethyl)-5-fluoro-N-methyl-1H-indole-2-carboxamide;
(R)-N-(1-(6,7-difluoro-1-oxo-1,2-dihydroisoquinolin-4-yl)ethyl)-6-fluoro-N-methyl-1H-indole-2-carboxamide;
(S)-N-(1-(6,7-difluoro-1-oxo-1,2-dihydroisoquinolin-4-yl)ethyl)-6-fluoro-N-methyl-1H-indole-2-carboxamide;
(R)-N-(1-(6,7-difluoro-1-oxo-1,2-dihydroisoquinolin-4-yl)ethyl)-7-fluoro-N-methyl-1H-indole-2-carboxamide;
(S)-N-(1-(6,7-difluoro-1-oxo-1,2-dihydroisoquinolin-4-yl)ethyl)-7-fluoro-N-methyl-1H-indole-2-carboxamide;
(R)-N-(1-(6,7-difluoro-1-oxo-1,2-dihydroisoquinolin-4-yl)ethyl)-5,6-difluoro-N-methyl-1H-indole-2-carboxamide;
(S)-N-(1-(6,7-difluoro-1-oxo-1,2-dihydroisoquinolin-4-yl)ethyl)-5,6-difluoro-N-methyl-1H-indole-2-carboxamide;
(R)-N-(1-(6,7-difluoro-1-oxo-1,2-dihydroisoquinolin-4-yl)ethyl)-4,6-difluoro-N-methyl-1H-indole-2-carboxamide;
(S)-N-(1-(6,7-difluoro-1-oxo-1,2-dihydroisoquinolin-4-yl)ethyl)-4,6-difluoro-N-methyl-1H-indole-2-carboxamide;
(3R)-N-((1R)-(6,7-difluoro-1-oxo-1,2-dihydroisoquinolin-4-yl)ethyl)-N-methyl-1,2,3,4-tetrahydroisoquinoline-3-carboxamide;
(3R)-N-((1S)-(6,7-difluoro-1-oxo-1,2-dihydroisoquinolin-4-yl)ethyl)-N-methyl-1,2,3,4-tetrahydroisoquinoline-3-carboxamide;
(3S)-N-((1R)-(6,7-difluoro-1-oxo-1,2-dihydroisoquinolin-4-yl)ethyl)-N-methyl-1,2,3,4-tetrahydroisoquinoline-3-carboxamide;
(3S)-N-((1S)-(6,7-difluoro-1-oxo-1,2-dihydroisoquinolin-4-yl)ethyl)-N-methyl-1,2,3,4-tetrahydroisoquinoline-3-carboxamide;
(2R)-N-((1R)-(6,7-difluoro-1-oxo-1,2-dihydroisoquinolin-4-yl)ethyl)-N-methylindoline-2-carboxamide;
(2R)-N-((1S)-(6,7-difluoro-1-oxo-1,2-dihydroisoquinolin-4-yl)ethyl)-N-methylindoline-2-carboxamide;
(2S)-N-((1R)-(6,7-difluoro-1-oxo-1,2-dihydroisoquinolin-4-yl)ethyl)-N-methylindoline-2-carboxamide;
(2S)-N-((1S)-(6,7-difluoro-1-oxo-1,2-dihydroisoquinolin-4-yl)ethyl)-N-methylindoline-2-carboxamide;
(R)-N-(1-(6,7-difluoro-1-oxo-1,2-dihydroisoquinolin-4-yl)ethyl)-N-methyl-1H-benzo[d]imidazole-2-carboxamide;
(S)-N-(1-(6,7-difluoro-1-oxo-1,2-dihydroisoquinolin-4-yl)ethyl)-N-methyl-1H-benzo[d]imidazole-2-carboxamide;
(R)-N-(1-(6,7-difluoro-1-oxo-1,2-dihydroisoquinolin-4-yl)ethyl)-N-methylbenzofuran-2-carboxamide;
(S)-N-(1-(6,7-difluoro-1-oxo-1,2-dihydroisoquinolin-4-yl)ethyl)-N-methylbenzofuran-2-carboxamide;
(R)-N-(1-(6,7-difluoro-1-oxo-1,2-dihydroisoquinolin-4-yl)ethyl)-N-(3-hydroxypropyl)-1H-indole-2-carboxamide;
(S)-N-(1-(6,7-difluoro-1-oxo-1,2-dihydroisoquinolin-4-yl)ethyl)-N-(3-hydroxypropyl)-1H-indole-2-carboxamide;
(R)-N-(1-(6,7-difluoro-1-oxo-1,2-dihydroisoquinolin-4-yl)ethyl)-4,5-difluoro-N-methyl-1H-indole-2-carboxamide;
(S)-N-(1-(6,7-difluoro-1-oxo-1,2-dihydroisoquinolin-4-yl)ethyl)-4,5-difluoro-N-methyl-1H-indole-2-carboxamide;
(2R)-N-((1R)-(6,7-difluoro-1-oxo-1,2-dihydroisoquinolin-4-yl)ethyl)-N-methylindoline-2-carboxamide;
(2R)-N-((1S)-(6,7-difluoro-1-oxo-1,2-dihydroisoquinolin-4-yl)ethyl)-N-methylindoline-2-carboxamide;
(2S)-N-((1R)-(6,7-difluoro-1-oxo-1,2-dihydroisoquinolin-4-yl)ethyl)-N-methylindoline-2-carboxamide;
(2S)-N-((1S)-(6,7-difluoro-1-oxo-1,2-dihydroisoquinolin-4-yl)ethyl)-N-methylindoline-2-carboxamide;
(3R)-N-((1R)-(6,7-difluoro-1-oxo-1,2-dihydroisoquinolin-4-yl)ethyl)-N-methyl-1,2,3,4-tetrahydroisoquinoline-3-carboxamide;
(3R)-N-((1S)-(6,7-difluoro-1-oxo-1,2-dihydroisoquinolin-4-yl)ethyl)-N-methyl-1,2,3,4-tetrahydroisoquinoline-3-carboxamide;
(3S)-N-((1R)-(6,7-difluoro-1-oxo-1,2-dihydroisoquinolin-4-yl)ethyl)-N-methyl-1,2,3,4-tetrahydroisoquinoline-3-carboxamide;
(3S)-N-((1S)-(6,7-difluoro-1-oxo-1,2-dihydroisoquinolin-4-yl)ethyl)-N-methyl-1,2,3,4-tetrahydroisoquinoline-3-carboxamide;
(R)-N-(1-(6,7-difluoro-1-oxo-1,2-dihydroisoquinolin-4-yl)ethyl)-N-methyl-4,5,6,7-tetrahydro-1H-indole-2-carboxamide;
(S)-N-(1-(6,7-difluoro-1-oxo-1,2-dihydroisoquinolin-4-yl)ethyl)-N-methyl-4,5,6,7-tetrahydro-1H-indole-2-carboxamide;
(R)-N-(1-(6,7-difluoro-1-oxo-1,2-dihydroisoquinolin-4-yl)ethyl)-N,1-dimethyl-1H-indole-2-carboxamide;
(S)-N-(1-(6,7-difluoro-1-oxo-1,2-dihydroisoquinolin-4-yl)ethyl)-N,1-dimethyl-1H-indole-2-carboxamide;
(R)-N-(1-(6,7-difluoro-1-oxo-1,2-dihydroisoquinolin-4-yl)ethyl)-N-methylimidazo[1,2-a]pyridine-2-carboxamide;
(S)-N-(1-(6,7-difluoro-1-oxo-1,2-dihydroisoquinolin-4-yl)ethyl)-N-methylimidazo[1,2-a]pyridine-2-carboxamide;
(R)-N-(1-(6,7-difluoro-1-oxo-1,2-dihydroisoquinolin-4-yl)ethyl)-N-methyl-1,4,5,6-tetrahydrocyclopenta[b]pyrrole-2-carboxamide;
(S)-N-(1-(6,7-difluoro-1-oxo-1,2-dihydroisoquinolin-4-yl)ethyl)-N-methyl-1,4,5,6-tetrahydrocyclopenta[b]pyrrole-2-carboxamide;
(R)-N-(1-(6,7-difluoro-1-oxo-1,2-dihydroisoquinolin-4-yl)ethyl)-5,6-difluoro-N-methyl-1H-benzo[d]imidazole-2-carboxamide;
(S)-N-(1-(6,7-difluoro-1-oxo-1,2-dihydroisoquinolin-4-yl)ethyl)-5,6-difluoro-N-methyl-1H-benzo[d]imidazole-2-carboxamide;
(R)-N-(1-(6,7-difluoro-1-oxo-1,2-dihydroisoquinolin-4-yl)ethyl)-N-methyl-1H-pyrrolo[2,3-b]pyridine-2-carboxamide;
(S)-N-(1-(6,7-difluoro-1-oxo-1,2-dihydroisoquinolin-4-yl)ethyl)-N-methyl-1H-pyrrolo[2,3-b]pyridine-2-carboxamide;
(R)-N-(1-(6,7-difluoro-1-oxo-1,2-dihydroisoquinolin-4-yl)ethyl)-N-methyl-1H-indole-3-carboxamide;
(S)-N-(1-(6,7-difluoro-1-oxo-1,2-dihydroisoquinolin-4-yl)ethyl)-N-methyl-1H-indole-3-carboxamide;
(R)-N-(1-(6,7-difluoro-1-oxo-1,2-dihydroisoquinolin-4-yl)ethyl)-N-methyl-1H-indazole-3-carboxamide;
(S)-N-(1-(6,7-difluoro-1-oxo-1,2-dihydroisoquinolin-4-yl)ethyl)-N-methyl-1H-indazole-3-carboxamide;
(R)-N-(1-(7,8-difluoro-1-oxo-1,2-dihydroisoquinolin-4-yl)ethyl)-N-methyl-1,4,5,6-tetrahydrocyclopenta[b]pyrrole-2-carboxamide;
(S)-N-(1-(7,8-difluoro-1-oxo-1,2-dihydroisoquinolin-4-yl)ethyl)-N-methyl-1,4,5,6-tetrahydrocyclopenta[b]pyrrole-2-carboxamide;
(R)-N-(1-(6,7-difluoro-1-oxo-1,2-dihydroisoquinolin-4-yl)ethyl)-2-(1H-indol-2-yl)-N-methylacetamide;
(S)-N-(1-(6,7-difluoro-1-oxo-1,2-dihydroisoquinolin-4-yl)ethyl)-2-(1H-indol-2-yl)-N-methylacetamide;
(R)-3-chloro-N-(1-(6,7-difluoro-1-oxo-1,2-dihydroisoquinolin-4-yl)ethyl)-N-methyl-1H-indole-2-carboxamide;
(S)-3-chloro-N-(1-(6,7-difluoro-1-oxo-1,2-dihydroisoquinolin-4-yl)ethyl)-N-methyl-1H-indole-2-carboxamide;
N-((1R)-(6,7-difluoro-1-oxo-1,2-dihydroisoquinolin-4-yl)ethyl)-5-fluoro-N-methylindoline-(2R)-carboxamide;
N-((1R)-(6,7-difluoro-1-oxo-1,2-dihydroisoquinolin-4-yl)ethyl)-5-fluoro-N-methylindoline-(2S)-carboxamide;
N-((1S)-(6,7-difluoro-1-oxo-1,2-dihydroisoquinolin-4-yl)ethyl)-5-fluoro-N-methylindoline-(2R)-carboxamide;
N-((1S)-(6,7-difluoro-1-oxo-1,2-dihydroisoquinolin-4-yl)ethyl)-5-fluoro-N-methylindoline-(2S)-carboxamide;
N-((1R)-(7,8-difluoro-1-oxo-1,2-dihydroisoquinolin-4-yl)ethyl)-4,6-difluoro-N-methylindoline-(2R)-carboxamide;
N-((1R)-(7,8-difluoro-1-oxo-1,2-dihydroisoquinolin-4-yl)ethyl)-4,6-difluoro-N-methylindoline-(2S)-carboxamide;
N-((1S)-(7,8-difluoro-1-oxo-1,2-dihydroisoquinolin-4-yl)ethyl)-4,6-difluoro-N-methylindoline-(2R)-carboxamide;
N-((1S)-(7,8-difluoro-1-oxo-1,2-dihydroisoquinolin-4-yl)ethyl)-4,6-difluoro-N-methylindoline-(2S)-carboxamide;
(R)-N-(1-(6,7-difluoro-1-oxo-1,2-dihydroisoquinolin-4-yl)ethyl)-N-methylindolizine-2-carboxamide;
(S)-N-(1-(6,7-difluoro-1-oxo-1,2-dihydroisoquinolin-4-yl)ethyl)-N-methylindolizine-2-carboxamide;
(R)-N-(1-(7,8-difluoro-1-oxo-1,2-dihydroisoquinolin-4-yl)ethyl)-N-methylindolizine-2-carboxamide;
(S)-N-(1-(7,8-difluoro-1-oxo-1,2-dihydroisoquinolin-4-yl)ethyl)-N-methylindolizine-2-carboxamide;
N-((1R)-(7,8-difluoro-1-oxo-1,2-dihydroisoquinolin-4-yl)ethyl)-5-fluoro-N-methylindoline-(2R)-carboxamide;
N-((1R)-(7,8-difluoro-1-oxo-1,2-dihydroisoquinolin-4-yl)ethyl)-5-fluoro-N-methylindoline-(2S)-carboxamide;
N-((1S)-(7,8-difluoro-1-oxo-1,2-dihydroisoquinolin-4-yl)ethyl)-5-fluoro-N-methylindoline-(2R)-carboxamide;
N-((1S)-(7,8-difluoro-1-oxo-1,2-dihydroisoquinolin-4-yl)ethyl)-5-fluoro-N-methylindoline-(2S)-carboxamide;
(R)-8-chloro-N-(1-(6,7-difluoro-1-oxo-1,2-dihydroisoquinolin-4-yl)ethyl)-N-methylindolizine-2-carboxamide;
(S)-8-chloro-N-(1-(6,7-difluoro-1-oxo-1,2-dihydroisoquinolin-4-yl)ethyl)-N-methylindolizine-2-carboxamide;
(R)-N-(1-(6,7-difluoro-1-oxo-1,2-dihydroisoquinolin-4-yl)ethyl)-8-fluoro-N-methylindolizine-2-carboxamide;
(S)-N-(1-(6,7-difluoro-1-oxo-1,2-dihydroisoquinolin-4-yl)ethyl)-8-fluoro-N-methylindolizine-2-carboxamide;
(R)-N-(1-(7,8-difluoro-1-oxo-1,2-dihydroisoquinolin-4-yl)ethyl)-8-fluoro-N-methylindolizine-2-carboxamide;
(S)-N-(1-(7,8-difluoro-1-oxo-1,2-dihydroisoquinolin-4-yl)ethyl)-8-fluoro-N-methylindolizine-2-carboxamide;
(R)-N-(1-(6,7-difluoro-1-oxo-1,2-dihydroisoquinolin-4-yl)ethyl)-N-(3-hydroxypropyl)-2-(1H-indol-2-yl)acetamide;
(S)-N-(1-(6,7-difluoro-1-oxo-1,2-dihydroisoquinolin-4-yl)ethyl)-N-(3-hydroxypropyl)-2-(1H-indol-2-yl)acetamide;
(R)-N-(1-(7,8-difluoro-1-oxo-1,2-dihydroisoquinolin-4-yl)ethyl)-N-methyl-6-oxo-1,6-dihydropyridine-2-carboxamide;
(S)-N-(1-(7,8-difluoro-1-oxo-1,2-dihydroisoquinolin-4-yl)ethyl)-N-methyl-6-oxo-1,6-dihydropyridine-2-carboxamide;
(R)-N-(1-(6,7-difluoro-1-oxo-1,2-dihydroisoquinolin-4-yl)ethyl)-5,5-difluoro-N-methyl-4,5,6,7-tetrahydro-1H-indole-2-carboxamide;
(S)-N-(1-(6,7-difluoro-1-oxo-1,2-dihydroisoquinolin-4-yl)ethyl)-5,5-difluoro-N-methyl-4,5,6,7-tetrahydro-1H-indole-2-carboxamide;
(R)-N-(1-(7,8-difluoro-1-oxo-1,2-dihydroisoquinolin-4-yl)ethyl)-5,5-difluoro-N-methyl-4,5,6,7-tetrahydro-1H-indole-2-carboxamide;
(S)-N-(1-(7,8-difluoro-1-oxo-1,2-dihydroisoquinolin-4-yl)ethyl)-5,5-difluoro-N-methyl-4,5,6,7-tetrahydro-1H-indole-2-carboxamide;
(R)-8-chloro-N-(1-(7,8-difluoro-1-oxo-1,2-dihydroisoquinolin-4-yl)ethyl)-N-methylindolizine-2-carboxamide;
(S)-8-chloro-N-(1-(7,8-difluoro-1-oxo-1,2-dihydroisoquinolin-4-yl)ethyl)-N-methylindolizine-2-carboxamide;
(R)-3-chloro-N-(1-(6,7-difluoro-1-oxo-1,2-dihydroisoquinolin-4-yl)ethyl)-N-(3-hydroxypropyl)-1H-indole-2-carboxamide;
(S)-3-chloro-N-(1-(6,7-difluoro-1-oxo-1,2-dihydroisoquinolin-4-yl)ethyl)-N-(3-hydroxypropyl)-1H-indole-2-carboxamide;
(R)-7-chloro-N-(1-(6,7-difluoro-1-oxo-1,2-dihydroisoquinolin-4-yl)ethyl)-N-methylindolizine-2-carboxamide;
(S)-7-chloro-N-(1-(6,7-difluoro-1-oxo-1,2-dihydroisoquinolin-4-yl)ethyl)-N-methylindolizine-2-carboxamide;
N-((1R)-(6,7-difluoro-1-oxo-1,2-dihydroisoquinolin-4-yl)ethyl)-4,6-difluoro-N-methylindoline-(2R)-carboxamide;
N-((1R)-(6,7-difluoro-1-oxo-1,2-dihydroisoquinolin-4-yl)ethyl)-4,6-difluoro-N-methylindoline-(2S)-carboxamide;
N-((1S)-(6,7-difluoro-1-oxo-1,2-dihydroisoquinolin-4-yl)ethyl)-4,6-difluoro-N-methylindoline-(2R)-carboxamide;
N-((1S)-(6,7-difluoro-1-oxo-1,2-dihydroisoquinolin-4-yl)ethyl)-4,6-difluoro-N-methylindoline-(2S)-carboxamide;
(R)-N-(1-(6,7-difluoro-1-oxo-1,2-dihydroisoquinolin-4-yl)ethyl)-6-fluoro-N-methylindolizine-2-carboxamide;
(S)-N-(1-(6,7-difluoro-1-oxo-1,2-dihydroisoquinolin-4-yl)ethyl)-6-fluoro-N-methylindolizine-2-carboxamide;
N-((1R)-(7,8-difluoro-1-oxo-1,2-dihydroisoquinolin-4-yl)ethyl)-N,3,3-trimethylindoline-(2R)-carboxamide;
N-((1R)-(7,8-difluoro-1-oxo-1,2-dihydroisoquinolin-4-yl)ethyl)-N,3,3-trimethylindoline-(2S)-carboxamide;
N-((1S)-(7,8-difluoro-1-oxo-1,2-dihydroisoquinolin-4-yl)ethyl)-N,3,3-trimethylindoline-(2R)-carboxamide;
N-((1S)-(7,8-difluoro-1-oxo-1,2-dihydroisoquinolin-4-yl)ethyl)-N,3,3-trimethylindoline-(2S)-carboxamide;
(R)-N-(1-(6,7-difluoro-1-oxo-1,2-dihydroisoquinolin-4-yl)ethyl)-7-fluoro-N-methylindolizine-2-carboxamide;
(S)-N-(1-(6,7-difluoro-1-oxo-1,2-dihydroisoquinolin-4-yl)ethyl)-7-fluoro-N-methylindolizine-2-carboxamide;
(R)-6-chloro-N-(1-(6,7-difluoro-1-oxo-1,2-dihydroisoquinolin-4-yl)ethyl)-N-methylindolizine-2-carboxamide;
(S)-6-chloro-N-(1-(6,7-difluoro-1-oxo-1,2-dihydroisoquinolin-4-yl)ethyl)-N-methylindolizine-2-carboxamide;
N-((1R)-(6,7-difluoro-1-oxo-1,2-dihydroisoquinolin-4-yl)ethyl)-N,3,3-trimethylindoline-(2R)-carboxamide;
N-((1R)-(6,7-difluoro-1-oxo-1,2-dihydroisoquinolin-4-yl)ethyl)-N,3,3-trimethylindoline-(2S)-carboxamide;
N-((1S)-(6,7-difluoro-1-oxo-1,2-dihydroisoquinolin-4-yl)ethyl)-N,3,3-trimethylindoline-(2R)-carboxamide;
N-((1S)-(6,7-difluoro-1-oxo-1,2-dihydroisoquinolin-4-yl)ethyl)-N,3,3-trimethylindoline-(2S)-carboxamide;
(R)-N-(1-(6,7-Difluoro-1-oxo-1,2-dihydroisoquinolin-4-yl)ethyl)-N-isobutylindolizine-2-carboxamide;
(S)-N-(1-(6,7-Difluoro-1-oxo-1,2-dihydroisoquinolin-4-yl)ethyl)-N-isobutylindolizine-2-carboxamide;
(R)-N-(1-(6,7-difluoro-1-oxo-1,2-dihydroisoquinolin-4-yl)ethyl)-3-fluoro-N-isobutyl-4-(trifluoromethyl)benzamide;
(S)-N-(1-(6,7-difluoro-1-oxo-1,2-dihydroisoquinolin-4-yl)ethyl)-3-fluoro-N-isobutyl-4-(trifluoromethyl)benzamide;
(R)-N-(1-(6,7-Difluoro-1-oxo-1,2-dihydroisoquinolin-4-yl)ethyl)-3-fluoro-N-methyl-4-(trifluoromethyl) benzamide;
(S)-N-(1-(6,7-Difluoro-1-oxo-1,2-dihydroisoquinolin-4-yl)ethyl)-3-fluoro-N-methyl-4-(trifluoromethyl) benzamide;
(R)-4-Chloro-N-(1-(6,7-difluoro-1-oxo-1,2-dihydroisoquinolin-4-yl)ethyl)-3-fluoro-N-methylbenzamide;
(S)-4-Chloro-N-(1-(6,7-difluoro-1-oxo-1,2-dihydroisoquinolin-4-yl)ethyl)-3-fluoro-N-methylbenzamide;
(R)-N-(1-(6,7-Difluoro-1-oxo-1,2-dihydroisoquinolin-4-yl)ethyl)-3,4,5-trifluoro-N-methylbenzamide;
(S)-N-(1-(6,7-Difluoro-1-oxo-1,2-dihydroisoquinolin-4-yl)ethyl)-3,4,5-trifluoro-N-methylbenzamide;
(R)-N-(1-(6,7-Difluoro-1-oxo-1,2-dihydroisoquinolin-4-yl)ethyl)-3-(difluoromethyl)-N-methylbenzamide;
(S)-N-(1-(6,7-Difluoro-1-oxo-1,2-dihydroisoquinolin-4-yl)ethyl)-3-(difluoromethyl)-N-methylbenzamide;
(R)-N-(1-(6,7-Difluoro-1-oxo-1,2-dihydroisoquinolin-4-yl)ethyl)-N-methylbenzamide;
(S)-N-(1-(6,7-Difluoro-1-oxo-1,2-dihydroisoquinolin-4-yl)ethyl)-N-methylbenzamide;
(R)-N-(1-(6,7-Difluoro-1-oxo-1,2-dihydroisoquinolin-4-yl)ethyl)-N-methyl-4-(trifluoromethyl) benzamide;
(S)-N-(1-(6,7-Difluoro-1-oxo-1,2-dihydroisoquinolin-4-yl)ethyl)-N-methyl-4-(trifluoromethyl) benzamide;
(R)-4-Chloro-N-(1-(6,7-difluoro-1-oxo-1,2-dihydroisoquinolin-4-yl)ethyl)-N-methylbenzamide;
(S)-4-Chloro-N-(1-(6,7-difluoro-1-oxo-1,2-dihydroisoquinolin-4-yl)ethyl)-N-methylbenzamide;
(R)-N-(1-(6,7-Difluoro-1-oxo-1,2-dihydroisoquinolin-4-yl)ethyl)-3-(difluoromethyl)-4-fluoro-N-methylbenzamide;
(S)-N-(1-(6,7-Difluoro-1-oxo-1,2-dihydroisoquinolin-4-yl)ethyl)-3-(difluoromethyl)-4-fluoro-N-methylbenzamide;
(R)-3-Chloro-N-(1-(6,7-difluoro-1-oxo-1,2-dihydroisoquinolin-4-yl)ethyl)-4-fluoro-N-methylbenzamide;
(S)-3-Chloro-N-(1-(6,7-difluoro-1-oxo-1,2-dihydroisoquinolin-4-yl)ethyl)-4-fluoro-N-methylbenzamide;
(R)-N-(1-(6,7-Difluoro-1-oxo-1,2-dihydroisoquinolin-4-yl)ethyl)-3,4-difluoro-N-methylbenzamide;
(S)-N-(1-(6,7-Difluoro-1-oxo-1,2-dihydroisoquinolin-4-yl)ethyl)-3,4-difluoro-N-methylbenzamide;
(R)-N-(1-(6,7-Difluoro-1-oxo-1,2-dihydroisoquinolin-4-yl)ethyl)-N,1-dimethyl-1H-indole-5-carboxamide;
(S)-N-(1-(6,7-Difluoro-1-oxo-1,2-dihydroisoquinolin-4-yl)ethyl)-N,1-dimethyl-1H-indole-5-carboxamide;
(R)-N-(1-(6,7-Difluoro-1-oxo-1,2-dihydroisoquinolin-4-yl)ethyl)-N,1-dimethyl-1H-indole-6-carboxamide;
(S)-N-(1-(6,7-Difluoro-1-oxo-1,2-dihydroisoquinolin-4-yl)ethyl)-N,1-dimethyl-1H-indole-6-carboxamide;
(R)-N1-Cyclopropyl-N2-(1-(6,7-difluoro-1-oxo-1,2-dihydroisoquinolin-4-yl)ethyl)-N2-methyloxalamide;
(S)-N1-Cyclopropyl-N2-(1-(6,7-difluoro-1-oxo-1,2-dihydroisoquinolin-4-yl)ethyl)-N2-methyloxalamide;
(R)-N1-(3-Chloro-4-fluorophenyl)-N2-(1-(6,7-difluoro-1-oxo-1,2-dihydroisoquinolin-4-yl)ethyl)-N2-methyloxalamide;
(S)-N1-(3-Chloro-4-fluorophenyl)-N2-(1-(6,7-difluoro-1-oxo-1,2-dihydroisoquinolin-4-yl)ethyl)-N2-methyloxalamide;
(R)-N-(1-(7,8-difluoro-1-oxo-1,2-dihydroisoquinolin-4-yl)ethyl)-3-fluoro-N-isobutyl-4-(trifluoromethyl)benzamide;
(S)-N-(1-(7,8-difluoro-1-oxo-1,2-dihydroisoquinolin-4-yl)ethyl)-3-fluoro-N-isobutyl-4-(trifluoromethyl)benzamide;
(R)-3-fluoro-N-methyl-N-(1-(1-oxo-1,2-dihydroisoquinolin-4-yl)ethyl)-4-(trifluoromethyl)benzamide;
(S)-3-fluoro-N-methyl-N-(1-(1-oxo-1,2-dihydroisoquinolin-4-yl)ethyl)-4-(trifluoromethyl)benzamide;
(R)-4-bromo-N-methyl-N-(1-(1-oxo-1,2-dihydroisoquinolin-4-yl)ethyl)benzamide;
(S)-4-bromo-N-methyl-N-(1-(1-oxo-1,2-dihydroisoquinolin-4-yl)ethyl)benzamide;
(R)-N-methyl-N-(1-(1-oxo-1,2-dihydroisoquinolin-4-yl)ethyl)-4-(trifluoromethyl)benzamide;
(S)-N-methyl-N-(1-(1-oxo-1,2-dihydroisoquinolin-4-yl)ethyl)-4-(trifluoromethyl)benzamide;
(R)-4-chloro-3-fluoro-N-methyl-N-(1-(1-oxo-1,2-dihydroisoquinolin-4-yl)ethyl)benzamide;
(S)-4-chloro-3-fluoro-N-methyl-N-(1-(1-oxo-1,2-dihydroisoquinolin-4-yl)ethyl)benzamide;
(R)-3-chloro-4-fluoro-N-methyl-N-(1-(1-oxo-1,2-dihydroisoquinolin-4-yl)ethyl)benzamide;
(S)-3-chloro-4-fluoro-N-methyl-N-(1-(1-oxo-1,2-dihydroisoquinolin-4-yl)ethyl)benzamide;
(R)-4-bromo-3-fluoro-N-methyl-N-(1-(1-oxo-1,2-dihydroisoquinolin-4-yl)ethyl)benzamide;
(S)-4-bromo-3-fluoro-N-methyl-N-(1-(1-oxo-1,2-dihydroisoquinolin-4-yl)ethyl)benzamide;
(R)-2-(4-chlorophenyl)-N-methyl-N-(1-(1-oxo-1,2-dihydroisoquinolin-4-yl)ethyl)acetamide;
(S)-2-(4-chlorophenyl)-N-methyl-N-(1-(1-oxo-1,2-dihydroisoquinolin-4-yl)ethyl)acetamide;
(R)-N-methyl-N-(1-(1-oxo-1,2-dihydroisoquinolin-4-yl)ethyl)indolizine-2-carboxamide;
(S)-N-methyl-N-(1-(1-oxo-1,2-dihydroisoquinolin-4-yl)ethyl)indolizine-2-carboxamide;
(R)-7-fluoro-N-methyl-N-(1-(1-oxo-1,2-dihydroisoquinolin-4-yl)ethyl)indolizine-2-carboxamide;
(S)-7-fluoro-N-methyl-N-(1-(1-oxo-1,2-dihydroisoquinolin-4-yl)ethyl)indolizine-2-carboxamide;
(R)-8-chloro-N-methyl-N-(1-(1-oxo-1,2-dihydroisoquinolin-4-yl)ethyl)indolizine-2-carboxamide;
(S)-8-chloro-N-methyl-N-(1-(1-oxo-1,2-dihydroisoquinolin-4-yl)ethyl)indolizine-2-carboxamide;
(R)-N-methyl-N-(1-(1-oxo-1,2-dihydroisoquinolin-4-yl)ethyl)-1H-indole-2-carboxamide;
(S)-N-methyl-N-(1-(1-oxo-1,2-dihydroisoquinolin-4-yl)ethyl)-1H-indole-2-carboxamide;
(R)-4-fluoro-N-methyl-N-(1-(1-oxo-1,2-dihydroisoquinolin-4-yl)ethyl)-1H-indole-2-carboxamide;
(S)-4-fluoro-N-methyl-N-(1-(1-oxo-1,2-dihydroisoquinolin-4-yl)ethyl)-1H-indole-2-carboxamide;
(R)-5-fluoro-N-methyl-N-(1-(1-oxo-1,2-dihydroisoquinolin-4-yl)ethyl)-1H-indole-2-carboxamide;
(S)-5-fluoro-N-methyl-N-(1-(1-oxo-1,2-dihydroisoquinolin-4-yl)ethyl)-1H-indole-2-carboxamide;
(R)-4,6-difluoro-N-methyl-N-(1-(1-oxo-1,2-dihydroisoquinolin-4-yl)ethyl)-1H-indole-2-carboxamide;
(S)-4,6-difluoro-N-methyl-N-(1-(1-oxo-1,2-dihydroisoquinolin-4-yl)ethyl)-1H-indole-2-carboxamide;
(R)-5,6-difluoro-N-methyl-N-(1-(1-oxo-1,2-dihydroisoquinolin-4-yl)ethyl)-1H-indole-2-carboxamide;
(S)-5,6-difluoro-N-methyl-N-(1-(1-oxo-1,2-dihydroisoquinolin-4-yl)ethyl)-1H-indole-2-carboxamide;
(R)-N-methyl-N-((R)-1-(1-oxo-1,2-dihydroisoquinolin-4-yl)ethyl)indoline-2-carboxamide;
(S)-N-methyl-N-((R)-1-(1-oxo-1,2-dihydroisoquinolin-4-yl)ethyl)indoline-2-carboxamide;
(R)-4-bromo-N-(1-(6,7-difluoro-2-methyl-1-oxo-1,2-dihydroisoquinolin-4-yl)ethyl)-3-fluoro-N-methylbenzamide;
(S)-4-bromo-N-(1-(6,7-difluoro-2-methyl-1-oxo-1,2-dihydroisoquinolin-4-yl)ethyl)-3-fluoro-N-methylbenzamide;
(R)-4-bromo-N-(1-(6,7-difluoro-1-methoxyisoquinolin-4-yl)ethyl)-N-methylbenzamide;
(S)-4-bromo-N-(1-(6,7-difluoro-1-methoxyisoquinolin-4-yl)ethyl)-N-methylbenzamide;
(R)-N-(1-(6,7-difluoro-2-methyl-1-oxo-1,2-dihydroisoquinolin-4-yl)ethyl)-3-fluoro-N-methyl-4-(trifluoromethyl)benzamide;
(S)-N-(1-(6,7-difluoro-2-methyl-1-oxo-1,2-dihydroisoquinolin-4-yl)ethyl)-3-fluoro-N-methyl-4-(trifluoromethyl)benzamide;
(R)-4-bromo-N-(1-(6,7-difluoro-2-methyl-1-oxo-1,2-dihydroisoquinolin-4-yl)ethyl)-N-methylbenzamide;
(S)-4-bromo-N-(1-(6,7-difluoro-2-methyl-1-oxo-1,2-dihydroisoquinolin-4-yl)ethyl)-N-methylbenzamide;
(R)-N-(1-(6,7-difluoro-1-methoxyisoquinolin-4-yl)ethyl)-5,6-difluoro-N-methyl-1H-indole-2-carboxamide;
(S)-N-(1-(6,7-difluoro-1-methoxyisoquinolin-4-yl)ethyl)-5,6-difluoro-N-methyl-1H-indole-2-carboxamide;
(R)-N-(1-(6,7-difluoro-2-methyl-1-oxo-1,2-dihydroisoquinolin-4-yl)ethyl)-8-fluoro-N-methylindolizine-2-carboxamide;
(S)-N-(1-(6,7-difluoro-2-methyl-1-oxo-1,2-dihydroisoquinolin-4-yl)ethyl)-8-fluoro-N-methylindolizine-2-carboxamide;
(R)-N-(1-(6,7-difluoro-2-methyl-1-oxo-1,2-dihydroisoquinolin-4-yl)ethyl)-5,6-difluoro-N-methyl-1H-indole-2-carboxamide;
(S)-N-(1-(6,7-difluoro-2-methyl-1-oxo-1,2-dihydroisoquinolin-4-yl)ethyl)-5,6-difluoro-N-methyl-1H-indole-2-carboxamide;
(R)-N-(1-(6,7-difluoro-1-methoxyisoquinolin-4-yl)ethyl)-3-fluoro-N-methyl-4-(trifluoromethyl)benzamide;
(S)-N-(1-(6,7-difluoro-1-methoxyisoquinolin-4-yl)ethyl)-3-fluoro-N-methyl-4-(trifluoromethyl)benzamide;
(R)-4-bromo-N-(1-(6,7-difluoro-1-methoxyisoquinolin-4-yl)ethyl)-3-fluoro-N-methylbenzamide;
(S)-4-bromo-N-(1-(6,7-difluoro-1-methoxyisoquinolin-4-yl)ethyl)-3-fluoro-N-methylbenzamide;
(R)-N-(1-(6,7-difluoro-1-methoxyisoquinolin-4-yl)ethyl)-8-fluoro-N-methylindolizine-2-carboxamide;
(S)-N-(1-(6,7-difluoro-1-methoxyisoquinolin-4-yl)ethyl)-8-fluoro-N-methylindolizine-2-carboxamide;
(2S)-N-(1-(6,7-difluoro-1-methoxyisoquinolin-4-yl)ethyl)-N-methylindoline-2-carboxamide;
(S)-N-((S)-1-(6,7-difluoro-1-methoxyisoquinolin-4-yl)ethyl)-N-methylindoline-2-carboxamide;
(S)-N-((R)-1-(6,7-difluoro-1-methoxyisoquinolin-4-yl)ethyl)-N-methylindoline-2-carboxamide;
(2R)-N-(1-(6,7-difluoro-1-methoxyisoquinolin-4-yl)ethyl)-N-methylindoline-2-carboxamide;
(R)-N-((S)-1-(6,7-difluoro-1-methoxyisoquinolin-4-yl)ethyl)-N-methylindoline-2-carboxamide;
(R)-N-((R)-1-(6,7-difluoro-1-methoxyisoquinolin-4-yl)ethyl)-N-methylindoline-2-carboxamide;
(2S)-N-(1-(6,7-difluoro-2-methyl-1-oxo-1,2-dihydroisoquinolin-4-yl)ethyl)-N-methylindoline-2-carboxamide;
(S)-N-((S)-1-(6,7-difluoro-2-methyl-1-oxo-1,2-dihydroisoquinolin-4-yl)ethyl)-N-methylindoline-2-carboxamide;
(S)-N-((R)-1-(6,7-difluoro-2-methyl-1-oxo-1,2-dihydroisoquinolin-4-yl)ethyl)-N-methylindoline-2-carboxamide;
(2R)-N-(1-(6,7-difluoro-2-methyl-1-oxo-1,2-dihydroisoquinolin-4-yl)ethyl)-N-methylindoline-2-carboxamide;
(R)-N-((S)-1-(6,7-difluoro-2-methyl-1-oxo-1,2-dihydroisoquinolin-4-yl)ethyl)-N-methylindoline-2-carboxamide;
(R)-N-((R)-1-(6,7-difluoro-2-methyl-1-oxo-1,2-dihydroisoquinolin-4-yl)ethyl)-N-methylindoline-2-carboxamide;
(R)-N-(1-(6,7-difluoro-1-oxo-1,2-dihydroisoquinolin-4-yl)ethyl)-N-methylindolizine-6-carboxamide;
(S)-N-(1-(6,7-difluoro-1-oxo-1,2-dihydroisoquinolin-4-yl)ethyl)-N-methylindolizine-6-carboxamide;
(R)-2-chloro-N-(1-(6,7-difluoro-1-oxo-1,2-dihydroisoquinolin-4-yl)ethyl)-N-methyl-4H-thieno[3,2-b]pyrrole-5-carboxamide;
(S)-2-chloro-N-(1-(6,7-difluoro-1-oxo-1,2-dihydroisoquinolin-4-yl)ethyl)-N-methyl-4H-thieno[3,2-b]pyrrole-5-carboxamide;
(R)-N-(1-(6,7-difluoro-1-oxo-1,2-dihydroisoquinolin-4-yl)ethyl)-N-methylindolizine-7-carboxamide;
(S)-N-(1-(6,7-difluoro-1-oxo-1,2-dihydroisoquinolin-4-yl)ethyl)-N-methylindolizine-7-carboxamide;
   or a salt, solvate, prodrug, isotopically labeled, stereoisomer, any mixture of stereoisomers, tautomer, and/or any mixture of tautomers thereof.

The compounds of the disclosure may possess one or more stereocenters, and each stereocenter may exist independently in either the (R)- or (S)-configuration. In certain embodiments, compounds described herein are present in optically active or racemic forms. The compounds described herein encompass racemic, optically active, regioisomeric and stereoisomeric forms, or combinations thereof that possess the therapeutically useful properties described herein. Preparation of optically active forms is achieved in any suitable manner, including, by way of non-limiting example, by resolution of the racemic form with recrystallization techniques, synthesis from optically active starting materials, chiral synthesis, or chromatographic separation using a chiral stationary phase. A compound illustrated herein by the racemic formula further represents either of the two enantiomers or any mixtures thereof, or in the case where two or more chiral centers are present, all diastereomers or any mixtures thereof.

In certain embodiments, the compounds of the disclosure exist as tautomers. All tautomers are included within the scope of the compounds recited herein.

Compounds described herein also include isotopically labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I ¹²⁵I ¹³N, ¹⁵N, ¹⁵O, ¹⁷O, ¹⁸O, ³²P, and ³⁵S. In certain embodiments, substitution with heavier isotopes such as deuterium affords greater chemical stability. Isotopically labeled compounds are prepared by any suitable method or by processes using an appropriate isotopically labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

In all of the embodiments provided herein, examples of suitable optional substituents are not intended to limit the scope of the claimed disclosure. The compounds of the disclosure may contain any of the substituents, or combinations of substituents, provided herein.

### Salts

The compounds described herein may form salts with acids or bases, and such salts are included in the present disclosure. The term “salts” embraces addition salts of free acids or bases that are useful within the methods of the disclosure. The term “pharmaceutically acceptable salt” refers to salts that possess toxicity profiles within a range that affords utility in pharmaceutical applications. In certain embodiments, the salts are pharmaceutically acceptable salts. Pharmaceutically unacceptable salts may nonetheless possess properties such as high crystallinity, which have utility in the practice of the present disclosure, such as for example utility in process of synthesis, purification or formulation of compounds useful within the methods of the disclosure.

Suitable pharmaceutically acceptable acid addition salts may be prepared from an inorganic acid or from an organic acid. Examples of inorganic acids include sulfate, hydrogen sulfate, hydrochloric, hydrobromic, hydriodic, nitric, carbonic, sulfuric, and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (or pamoic), methanesulfonic, ethanesulfonic, benzenesulfonic, pantothenic, sulfanilic, 2-hydroxyethanesulfonic, trifluoromethanesulfonic, p-toluenesulfonic, cyclohexylaminosulfonic, stearic, alginic, β-hydroxybutyric, salicylic, galactaric, galacturonic acid, glycerophosphonic acids and saccharin (e.g., saccharinate, saccharate). Salts may be comprised of a fraction of one, one or more than one molar equivalent of acid or base with respect to any compound of the disclosure.

Suitable pharmaceutically acceptable base addition salts of compounds of the disclosure include, for example, ammonium salts and metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (or N-methylglucamine) and procaine. All of these salts may be prepared from the corresponding compound by reacting, for example, the appropriate acid or base with the compound.

### Combination Therapies

In one aspect, the compounds of the disclosure are useful within the methods of the disclosure in combination with one or more additional agents useful for treating, ameliorating, and/or preventing HBV and/or HDV infections. These additional agents may comprise compounds or compositions identified herein, or compounds (e.g., commercially available compounds) known to treat, prevent, or reduce the symptoms of HBV and/or HDV infections.

Non-limiting examples of one or more additional agents useful for treating, ameliorating, and/or preventing HBV and/or HDV infections include: (a) reverse transcriptase inhibitors; (b) capsid inhibitors; (c) cccDNA formation inhibitors; (d) RNA destabilizers; (e) oligomeric nucleotides targeted against the HBV genome; (f) immunostimulators, such as checkpoint inhibitors (e.g., PD-L1 inhibitors); (g) GalNAc-siRNA conjugates targeted against an HBV gene transcript; and (h) therapeutic vaccine.

(a) Reverse Transcriptase Inhibitors

In certain embodiments, the reverse transcriptase inhibitor is a reverse-transcriptase inhibitor (NARTI or NRTI). In other embodiments, the reverse transcriptase inhibitor is a nucleotide analog reverse-transcriptase inhibitor (NtARTI or NtRTI).

Reported reverse transcriptase inhibitors include, but are not limited to, entecavir, clevudine, telbivudine, lamivudine, adefovir, and tenofovir, tenofovir disoproxil, tenofovir alafenamide, adefovir dipovoxil, (1R,2R,3R,5R)-3-(6-amino-9H-9-purinyl)-2-fluoro-5-(hydroxymethyl)-4-methylenecyclopentan-1-ol (described in U.S. Pat. No. 8,816,074, incorporated herein in its entirety by reference), emtricitabine, abacavir, elvucitabine, ganciclovir, lobucavir, famciclovir, penciclovir, and amdoxovir.

Reported reverse transcriptase inhibitors further include, but are not limited to, entecavir, lamivudine, and (1R,2R,3R,5R)-3-(6-amino-9H-9-purinyl)-2-fluoro-5-(hydroxymethyl)-4-methylenecyclopentan-1-ol.

Reported reverse transcriptase inhibitors further include, but are not limited to, a covalently bound phosphoramidate or phosphonamidate moiety of the above-mentioned reverse transcriptase inhibitors, or as described in for example U.S. Pat. No. 8,816,074, US Patent Application Publications No. US 2011/0245484 A1, and US 2008/0286230A1, all of which incorporated herein in their entireties by reference.

Reported reverse transcriptase inhibitors further include, but are not limited to, nucleotide analogs that comprise a phosphoramidate moiety, such as, for example, methyl ((((1R,3R,4R,5R)-3-(6-amino-9H-purin-9-yl)-4-fluoro-5-hydroxy-2-methylenecyclopentyl) methoxy)(phenoxy) phosphoryl)-(D or L)-alaninate and methyl ((((1R,2R,3R,4R)-3-fluoro-2-hydroxy-5-methylene-4-(6-oxo-1,6-dihydro-9H-purin-9-yl)cyclopentyl)methoxy)(phenoxy) phosphoryl)-(D or L)-alaninate. Also included are the individual diastereomers thereof, which include, for example, methyl ((R)-(((1R,3R,4R,5R)-3-(6-amino-9H-purin-9-yl)-4-fluoro-5-hydroxy-2-methylenecyclopentyl)methoxy)(phenoxy)phosphoryl)-(D or L)-alaninate and methyl ((S)-(((1R,3R,4R,5R)-3-(6-amino-9H-purin-9-yl)-4-fluoro-5-hydroxy-2-methylenecyclopentyl) methoxy)(phenoxy)phosphoryl)-(D or L)-alaninate.

Reported reverse transcriptase inhibitors further include, but are not limited to, compounds comprising a phosphonamidate moiety, such as, for example, tenofovir alafenamide, as well as those described in U.S. Patent Application Publication No. US 2008/0286230 A1, incorporated herein in its entirety by reference. Methods for preparing stereoselective phosphoramidate or phosphonamidate containing actives are described in, for example, U.S. Pat. No. 8,816,074, as well as U.S. Patent Application Publications No. US 2011/0245484 A1 and US 2008/0286230 A1, all of which incorporated herein in their entireties by reference.

(b) Capsid Inhibitors

As described herein, the term “capsid inhibitor” includes compounds that are capable of inhibiting the expression and/or function of a capsid protein either directly or indirectly. For example, a capsid inhibitor may include, but is not limited to, any compound that inhibits capsid assembly, induces formation of non-capsid polymers, promotes excess capsid assembly or misdirected capsid assembly, affects capsid stabilization, and/or inhibits encapsidation of RNA (pgRNA). Capsid inhibitors also include any compound that inhibits capsid function in a downstream event(s) within the replication process (e.g., viral DNA synthesis, transport of relaxed circular DNA (rcDNA) into the nucleus, covalently closed circular DNA (cccDNA) formation, virus maturation, budding and/or release, and the like). For example, in certain embodiments, the inhibitor detectably inhibits the expression level or biological activity of the capsid protein as measured, e.g., using an assay described herein. In certain embodiments, the inhibitor inhibits the level of rcDNA and downstream products of viral life cycle by at least 5%, at least 10%, at least 20%, at least 50%, at least 75%, or at least 90%.

Reported capsid inhibitors include, but are not limited to, compounds described in International Patent Applications Publication Nos WO 2013006394, WO 2014106019, and WO2014089296, all of which incorporated herein in their entireties by reference.

Reported capsid inhibitors also include, but are not limited to, the following compounds and pharmaceutically acceptable salts and/or solvates thereof: Bay-41-4109 (see Int'l Patent Application Publication No. WO 2013144129), AT-61 (see Int'l Patent Application Publication No. WO 1998033501; and King, et al., 1998, Antimicrob. Agents Chemother. 42(12):3179-3186), DVR-01 and DVR-23 (see Int'l Patent Application Publication No. WO 2013006394; and Campagna, et al., 2013, J. Virol. 87(12):6931, all of which incorporated herein in their entireties by reference.

In addition, reported capsid inhibitors include, but are not limited to, those generally and specifically described in U.S. Patent Application Publication Nos. US 2015/0225355, US 2015/0132258, US 2016/0083383, US 2016/0052921, US 2019/0225593, and Int'l Patent Application Publication Nos. WO 2013096744, WO 2014165128, WO 2014033170, WO 2014033167, WO 2014033176, WO 2014131847, WO 2014161888, WO 2014184350, WO 2014184365, WO 2015059212, WO 2015011281, WO 2015118057, WO 2015109130, WO 2015073774, WO 2015180631, WO 2015138895, WO 2016089990, WO 2017015451, WO 2016183266, WO 2017011552, WO 2017048950, WO2017048954, WO 2017048962, WO 2017064156, WO 2018052967, WO 2018172852, WO 2020023710, WO 2020123674 and are incorporated herein in their entirety by reference.

(c) cccDNA Formation Inhibitors

Covalently closed circular DNA (cccDNA) is generated in the cell nucleus from viral rcDNA and serves as the transcription template for viral mRNAs. As described herein, the term “cccDNA formation inhibitor” includes compounds that are capable of inhibiting the formation and/or stability of cccDNA either directly or indirectly. For example, a cccDNA formation inhibitor may include, but is not limited to, any compound that inhibits capsid disassembly, rcDNA entry into the nucleus, and/or the conversion of rcDNA into cccDNA. For example, in certain embodiments, the inhibitor detectably inhibits the formation and/or stability of the cccDNA as measured, e.g., using an assay described herein. In certain embodiments, the inhibitor inhibits the formation and/or stability of cccDNA by at least 5%, at least 10%, at least 20%, at least 50%, at least 75%, or at least 90%.

Reported cccDNA formation inhibitors include, but are not limited to, compounds described in Int'l Patent Application Publication No. WO 2013130703, and are incorporated herein in their entirety by reference.

In addition, reported cccDNA formation inhibitors include, but are not limited to, those generally and specifically described in U.S. Patent Application Publication No. US 2015/0038515 A1, and are incorporated herein in their entirety by reference.

(d) RNA Destabilizer

As used herein, the term “RNA destabilizer” refers to a molecule, or a salt or solvate thereof, that reduces the total amount of HBV RNA in mammalian cell culture or in a live human subject. In a non-limiting example, an RNA destabilizer reduces the amount of the RNA transcript(s) encoding one or more of the following HBV proteins: surface antigen, core protein, RNA polymerase, and e antigen. In certain embodiments, the RNA destabilizer reduces the total amount of HBV RNA in mammalian cell culture or in a live human subject by at least 5%, at least 10%, at least 20%, at least 50%, at least 75%, or at least 90%.

Reported RNA destabilizers include compounds described in U.S. Pat. No. 8,921,381, as well as compounds described in U.S. Patent Application Publication Nos. US 2015/0087659 and US 2013/0303552, all of which are incorporated herein in their entireties by reference.

In addition, reported RNA destabilizers include, but are not limited to, those generally and specifically described in Int'l Patent Application Publication Nos. WO 2015113990, WO 2015173164, US 2016/0122344, WO 2016107832, WO 2016023877, WO 2016128335, WO 2016177655, WO 2016071215, WO 2017013046, WO 2017016921, WO 2017016960, WO 2017017042, WO 2017017043, WO 2017102648, WO 2017108630, WO 2017114812, WO 2017140821, WO 2018085619, WO 2019177937, WO 2019222238, WO 2020150366, WO 2021025976 and are incorporated herein in their entirety by reference.

(e) Oligomeric Nucleotides Targeted Against the HBV Genome

Reported oligomeric nucleotides targeted against the HBV genome include, but are not limited to, Arrowhead-ARC-520 (see U.S. Pat. No. 8,809,293; and Wooddell et al., 2013, Molecular Therapy 21(5):973-985, all of which incorporated herein in their entireties by reference).

In certain embodiments, the oligomeric nucleotides can be designed to target one or more genes and/or transcripts of the HBV genome. Oligomeric nucleotide targeted to the HBV genome also include, but are not limited to, isolated, double stranded, siRNA molecules, that each include a sense strand and an antisense strand that is hybridized to the sense strand. In certain embodiments, the siRNA target one or more genes and/or transcripts of the HBV genome.

(f) Immunostimulators

Checkpoint Inhibitors

As described herein, the term “checkpoint inhibitor” includes any compound that is capable of inhibiting immune checkpoint molecules that are regulators of the immune system (e.g., stimulate or inhibit immune system activity). For example, some checkpoint inhibitors block inhibitory checkpoint molecules, thereby stimulating immune system function, such as stimulation of T cell activity against cancer cells. A non-limiting example of a checkpoint inhibitor is a PD-L1 inhibitor.

As described herein, the term “PD-L1 inhibitor” includes any compound that is capable of inhibiting the expression and/or function of the protein Programmed Death-Ligand 1 (PD-L1) either directly or indirectly. PD-L1, also known as cluster of differentiation 274 (CD274) or B7 homolog 1 (B7-H1), is a type 1 transmembrane protein that plays a major role in suppressing the adaptive arm of immune system during pregnancy, tissue allograft transplants, autoimmune disease, and hepatitis. PD-L1 binds to its receptor, the inhibitory checkpoint molecule PD-1 (which is found on activated T cells, B cells, and myeloid cells) so as to modulate activation or inhibition of the adaptive arm of immune system. In certain embodiments, the PD-L1 inhibitor inhibits the expression and/or function of PD-L1 by at least 5%, at least 10%, at least 20%, at least 50%, at least 75%, or at least 90%.

Reported PD-L1 inhibitors include, but are not limited to, compounds recited in one of the following patent application publications: US 2018/0057455; US 2018/0057486; WO 2017/106634; WO 2018/026971; WO 2018/045142; WO 2018/118848; WO 2018/119221; WO 2018/119236; WO 2018/119266; WO 2018/119286; WO 2018/121560; WO 2019/076343; WO 2019/087214; and are incorporated herein in their entirety by reference.

(g) GalNAc-siRNA Conjugates Targeted Against an HBV Gene Transcript

“GalNAc” is the abbreviation for N-acetylgalactosamine, and “siRNA” is the abbreviation for small interfering RNA. An siRNA that targets an HBV gene transcript is covalently bonded to GalNAc in a GalNAc-siRNA conjugate useful in the practice of the present disclosure. While not wishing to be bound by theory, it is believed that GalNAc binds to asialoglycoprotein receptors on hepatocytes thereby facilitating the targeting of the siRNA to the hepatocytes that are infected with HBV. The siRNA enter the infected hepatocytes and stimulate destruction of HBV gene transcripts by the phenomenon of RNA interference.

Examples of GalNAc-siRNA conjugates useful in the practice of this aspect of the present disclosure are set forth in published international application PCT/CA2017/050447 (PCT Application Publication number WO/2017/177326, published on Oct. 19, 2017) and PCT/US2018/0226918 (PCT Application Publication number WO/2018/191278, published on Oct. 18, 2018), all of which are hereby incorporated by reference in their entireties.

(h) Therapeutic Vaccines

In certain embodiments, administration of a therapeutic vaccine is useful in the practice of the present disclosure for the treatment of a viral disease in a subject. In certain embodiments, the viral disease is a hepatitis virus. In certain embodiments, the hepatitis virus is at least one selected from the group consisting of hepatitis B virus (HBV) and hepatitis D virus (HDV). In certain embodiments, the subject is a human. A synergistic effect may be calculated, for example, using suitable methods such as, for example, the Sigmoid-Eₘₐₓ equation (Holford & Scheiner, 1981, Clin. Pharmacokinet. 6:429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22:27-55). Each equation referred to elsewhere herein may be applied to experimental data to generate a corresponding graph to aid in assessing the effects of the drug combination. The corresponding graphs associated with the equations referred to elsewhere herein are the concentration-effect curve, isobologram curve and combination index curve, respectively.

### Synthesis

The present disclosure further provides methods of preparing compounds of the present disclosure. Compounds of the present teachings can be prepared in accordance with the procedures outlined herein, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field.

It is appreciated that where typical or preferred process conditions (i.e., reaction temperatures, times, mole ratios of reactants, solvents, pressures, and so forth) are given, other process conditions can also be used unless otherwise stated. Optimum reaction conditions can vary with the particular reactants or solvent used, but such conditions can be determined by one skilled in the art by routine optimization procedures. Those skilled in the art of organic synthesis will recognize that the nature and order of the synthetic steps presented can be varied for the purpose of optimizing the formation of the compounds described herein.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatography such as high-performance liquid chromatograpy (HPLC), gas chromatography (GC), gel-permeation chromatography (GPC), or thin layer chromatography (TLC).

Preparation of the compounds can involve protection and deprotection of various chemical groups. The need for protection and deprotection and the selection of appropriate protecting groups can be readily determined by one skilled in the art. The chemistry of protecting groups can be found, for example, in Greene, et al., Protective Groups in Organic Synthesis, 2d. Ed. (Wiley & Sons, 1991), the entire disclosure of which is incorporated by reference herein for all purposes.

The reactions or the processes described herein can be carried out in suitable solvents that can be readily selected by one skilled in the art of organic synthesis. Suitable solvents typically are substantially nonreactive with the reactants, intermediates, and/or products at the temperatures at which the reactions are carried out, i.e., temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected.

A compound of formula (I) can be prepared from commercially available or previously documented starting materials, for example, according to the synthetic methods outlined in Scheme 1. (Un)substituted isoquinolin-1(2H)-ones (II) can either be commercially acquired or synthesized according to procedures outlined in, for example, Tetrahedron, 2002, 58:5761-5766. Bromination of II using, in non-limiting examples, pyridinium hydrobromide perbromide as described in J. Med Chem., 2014, 57:1299-1322 or N-bromosuccinimide as described in Angew. Chem. Int. Ed., 2011, 50:8416-8419, provides III. Chlorination of III with, in a non-limiting example, phosphorus oxychloride as described in Bioorg. Med. Chem. Lett., 2017, 27:217-222, followed by chloride displacement with, for example, an alcohol in the presence of a base (R⁸=OR′), in a non-limiting example, as exemplified in WO200472033, provides IV. Halogen metal exchange of the heteroaryl bromide of IV, followed by quenching of the resulting heteroaryl anion with a suitable electrophile such as, but not limited to, DMF, carbon dioxide, a dialkyl dicarbonate, an anhydride, an aldehyde, a ketone, and/or a Weinreb amide, or manipulation of the bromine using transition metal catalyzed coupling techniques, provides V. Reductive alkylation utilizing V subsequently provides VI. When V is an aldehyde or a ketone, reductive alkylation can be achieved by reacting that compound with a primary amine to form an imine, which is then reacted with a reducing agent, such as but not limited to sodium borohydride, or a carbon-based nucleophile, such as but not limited to a Grignard reagent or an alkyl/aryl lithium. Alternatively, when V is an aldehyde or a ketone, reductive alkylation can be achieved by reacting that compound with a primary sulfinamide to form a sulfinimine, which is subsequently reacted with a reducing agent, such as but not limited to sodium borohydride, or a carbon-based nucleophile, such as but not limited to a Grignard reagent or an alkyl/aryl lithium. In certain embodiments, the primary sulfinamide can be racemic, scalemic, or enantiopure, and can be used to influence the stereochemical outcome of the sulfinimine reduction. The stereochemical control of such reductions is detailed in WO 2020123674, which is hereby incorporated by reference in its entirety. The resulting secondary sulfinamide can be further functionalized with an electrophile, such as but not limited to an alkyl halide, in the presence of base, such as but not limited to sodium hydride, and the sulfinamido group can be deprotected to provide VI. Under certain conditions, sulfinamido deprotection can be concomitant with R′-dealkylation to provide VIII directly. Alternatively, when V is an aldehyde or a ketone, the compound can be reduced to the corresponding primary or secondary alcohol using a reducing agent, such as but not limited to sodium borohydride. The primary or secondary alcohol can be functionalized with, for example, para-toluene sulfonyl chloride to provide the corresponding tosylate, or converted to the alkyl halide, using for example thionyl chloride, and subsequently reacted with a primary amine to provide VI. Functionalization of VI with a variety of electrophiles, for example an activated carboxylic acid or an acid chloride, provides VII. Alternatively, acid mediated O-dealkylation of VI (R⁸═OR′), using for example hydrochloric or hydrobromic acid, provides VIII, which can be functionalized with a variety of electrophiles, for example an activated carboxylic acid or an acid chloride, to provide IX.

Alternatively, ketone XX can be synthesized from bromoisoquinolinone III via palladium catalyzed coupling with a vinyl stannane followed by hydrolysis of the resulting enol ether. Reductive alkylation utilizing a primary amine can subsequently provide VIII which can be functionalized to afford IX (Scheme 2). The protocols incorporated elsewhere herein exemplify synthesis of representative compounds of the present disclosure. Analogous compounds can be synthesized in a similar fashion to those exemplified using the appropriately substituted intermediates and reagents.

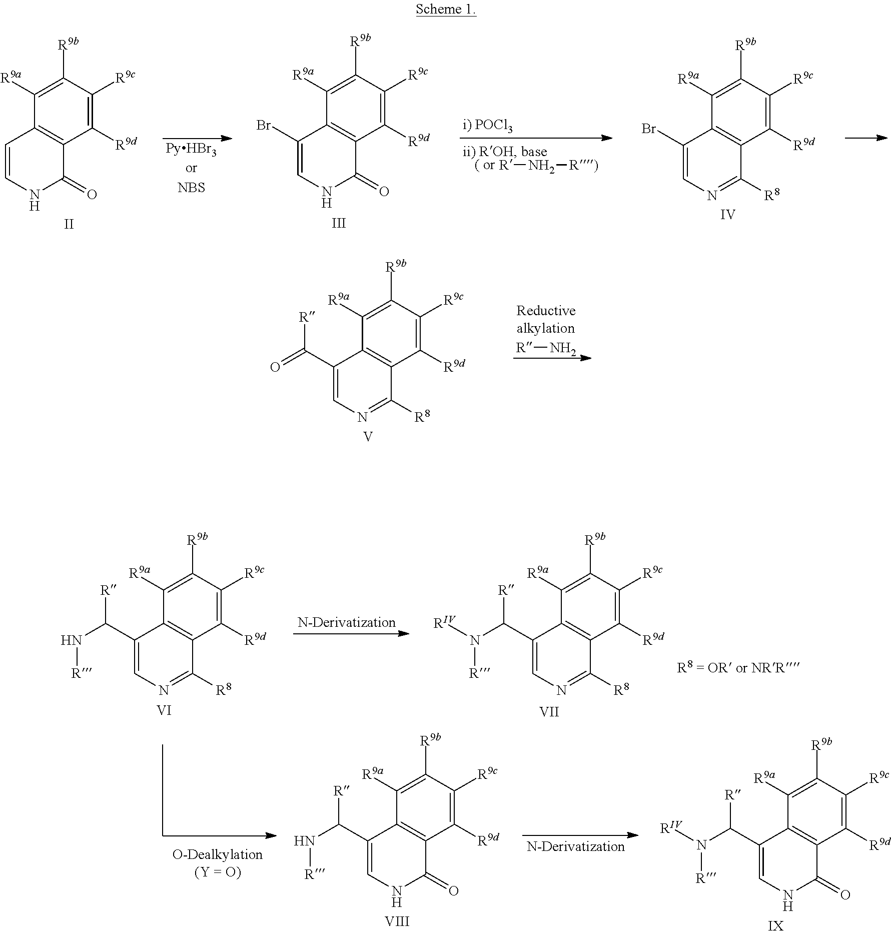

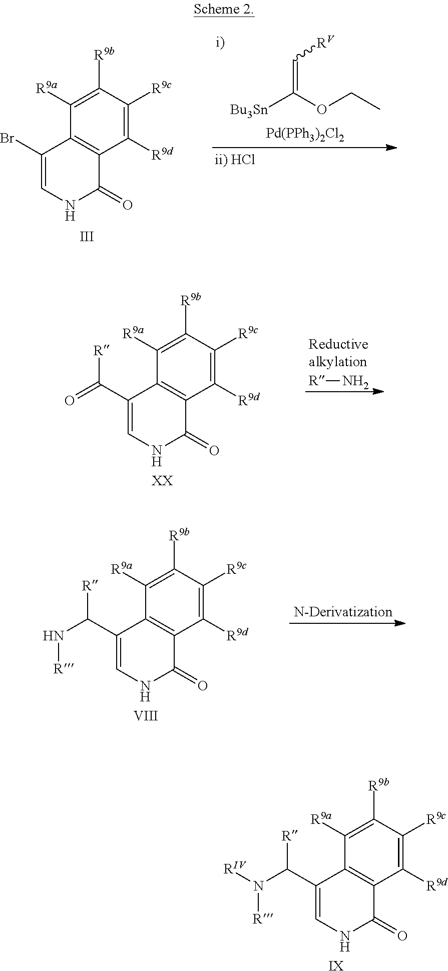

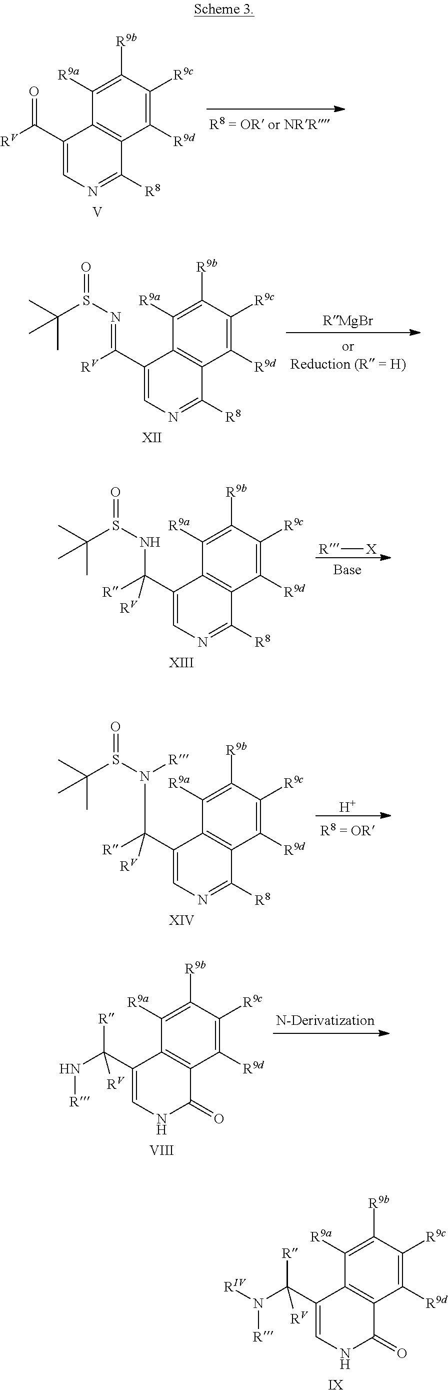

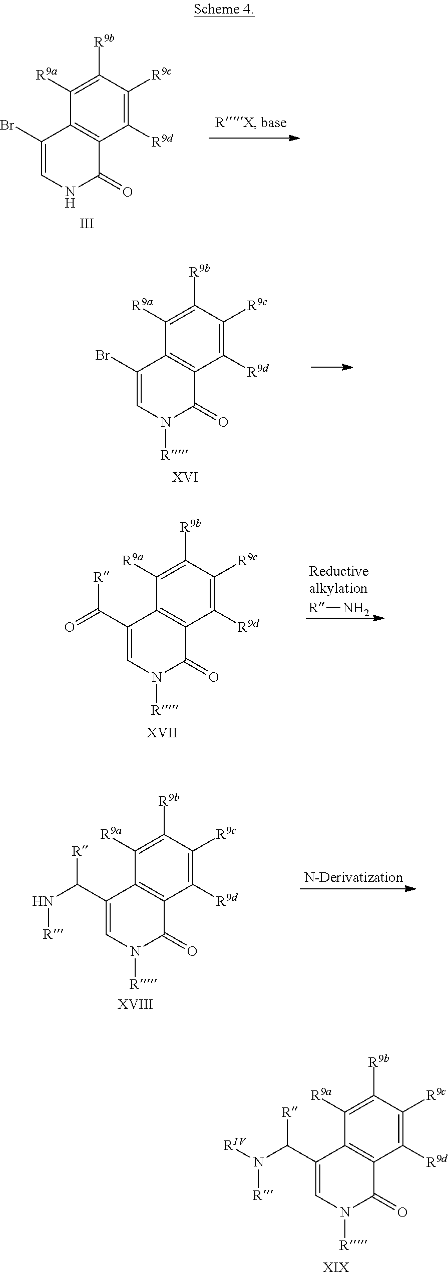

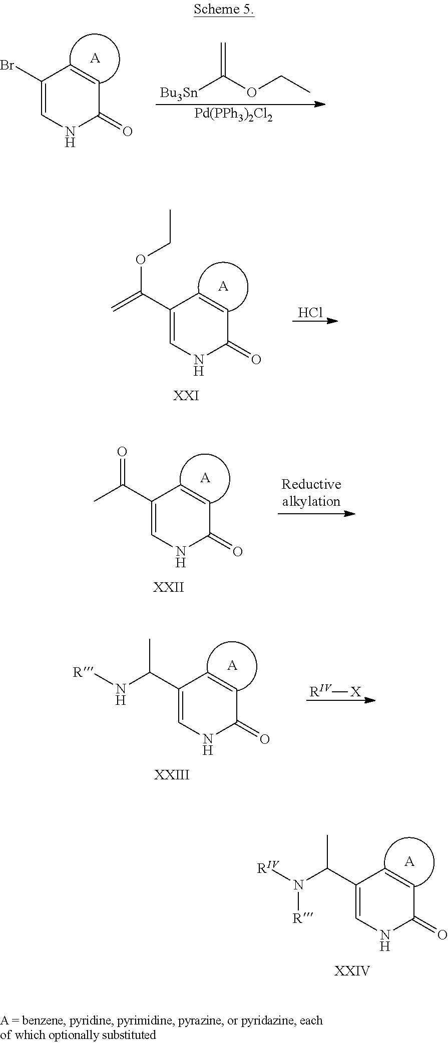

The protocols incorporated elsewhere herein exemplify synthesis of representative compounds of the present disclosure. Analogous compounds can be synthesized in a similar fashion to those exemplified using the appropriately substituted intermediates and reagents. The disclosures of PCT Application No. PCT/US2019/065756 filed Dec. 11, 2019, U.S. Provisional Applications No. 62/896,237 filed Sep. 5, 2019, and U.S. Provisional Application No. 62/778,471 filed Dec. 12, 2018, are incorporated herein by reference in their entireties.

### Methods

The disclosure provides a method of treating, ameliorating, and/or preventing hepatitis virus infection in a subject. In certain embodiments, the infection comprises hepatitis B virus (HBV) infection. In certain embodiments, the infection comprises hepatitis D virus (HDV) infection. In certain embodiments, the infection comprises HBV infection and HDV infection. In other embodiments, the method comprises administering to the subject in need thereof a therapeutically effective amount of at least one compound of the disclosure. In yet other embodiments, the at least one compound of the disclosure is the only antiviral agent administered to the subject. In yet other embodiments, the at least one compound is administered to the subject in a pharmaceutically acceptable composition. In yet other embodiments, the subject is further administered at least one additional agent useful for treating, ameliorating, and/or preventing the hepatitis infection. In yet other embodiments, the at least one additional agent comprises at least one selected from the group consisting of reverse transcriptase inhibitor; capsid inhibitor; cccDNA formation inhibitor; RNA destabilizer; oligomeric nucleotide targeted against the HBV genome; immunostimulator, such as checkpoint inhibitor (e.g., PD-L1 inhibitor); GalNAc-siRNA conjugate targeted against an HBV gene transcript; and therapeutic vaccine. In yet other embodiments, the subject is co-administered the at least one compound and the at least one additional agent. In yet other embodiments, the at least one compound and the at least one additional agent are coformulated.

The disclosure further provides a method of inhibiting expression and/or function of a viral capsid protein either directly or indirectly in a subject. In certain embodiments, the method comprises administering to the subject in need thereof a therapeutically effective amount of at least one compound of the disclosure. In other embodiments, the at least one compound is administered to the subject in a pharmaceutically acceptable composition. In yet other embodiments, the at least one compound of the disclosure is the only antiviral agent administered to the subject. In yet other embodiments, the subject is further administered at least one additional agent useful for treating, ameliorating, and/or preventing HBV infection. In yet other embodiments, the at least one additional agent comprises at least one selected from the group consisting of reverse transcriptase inhibitor; capsid inhibitor; cccDNA formation inhibitor; RNA destabilizer; oligomeric nucleotide targeted against the HBV genome; immunostimulator, such as checkpoint inhibitor (e.g., PD-L1 inhibitor); GalNAc-siRNA conjugate targeted against an HBV gene transcript; and therapeutic vaccine. In yet other embodiments, the subject is co-administered the at least one compound and the at least one additional agent. In yet other embodiments, the at least one compound and the at least one additional agent are coformulated.

In certain embodiments, the subject is a mammal. In other embodiments, the mammal is a human.

### Pharmaceutical Compositions and Formulations

The disclosure provides pharmaceutical compositions comprising at least one compound of the disclosure or a salt or solvate thereof, which are useful to practice methods of the disclosure. Such a pharmaceutical composition may consist of at least one compound of the disclosure or a salt or solvate thereof, in a form suitable for administration to a subject, or the pharmaceutical composition may comprise at least one compound of the disclosure or a salt or solvate thereof, and one or more pharmaceutically acceptable carriers, one or more additional ingredients, or any combinations of these. At least one compound of the disclosure may be present in the pharmaceutical composition in the form of a physiologically acceptable salt, such as in combination with a physiologically acceptable cation or anion, as is well known in the art.

In certain embodiments, the pharmaceutical compositions useful for practicing the method of the disclosure may be administered to deliver a dose of between 1 ng/kg/day and 100 mg/kg/day. In other embodiments, the pharmaceutical compositions useful for practicing the disclosure may be administered to deliver a dose of between 1 ng/kg/day and 1,000 mg/kg/day.

The relative amounts of the active ingredient, the pharmaceutically acceptable carrier, and any additional ingredients in a pharmaceutical composition of the disclosure will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the disclosure may be suitably developed for nasal, inhalational, oral, rectal, vaginal, pleural, peritoneal, parenteral, topical, transdermal, pulmonary, intranasal, buccal, ophthalmic, epidural, intrathecal, intravenous, or another route of administration. A composition useful within the methods of the disclosure may be directly administered to the brain, the brainstem, or any other part of the central nervous system of a mammal or bird. Other contemplated formulations include projected nanoparticles, microspheres, liposomal preparations, coated particles, polymer conjugates, resealed erythrocytes containing the active ingredient, and immunologically-based formulations.

In certain embodiments, the compositions of the disclosure are part of a pharmaceutical matrix, which allows for manipulation of insoluble materials and improvement of the bioavailability thereof, development of controlled or sustained release products, and generation of homogeneous compositions. By way of example, a pharmaceutical matrix may be prepared using hot melt extrusion, solid solutions, solid dispersions, size reduction technologies, molecular complexes (e.g., cyclodextrins, and others), microparticulate, and particle and formulation coating processes. Amorphous or crystalline phases may be used in such processes.

The route(s) of administration will be readily apparent to the skilled artisan and will depend upon any number of factors including the type and severity of the disease being treated, the type and age of the veterinary or human patient being treated, and the like.

The formulations of the pharmaceutical compositions described herein may be prepared by any method known or hereafter developed in the art of pharmacology and pharmaceutics. In general, such preparatory methods include the step of bringing the active ingredient into association with a carrier or one or more other accessory ingredients, and then, if necessary or desirable, shaping or packaging the product into a desired single-dose or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the disclosure is contemplated include, but are not limited to, humans and other primates, mammals including commercially relevant mammals such as cattle, pigs, horses, sheep, cats, and dogs.

In certain embodiments, the compositions of the disclosure are formulated using one or more pharmaceutically acceptable excipients or carriers. In certain embodiments, the pharmaceutical compositions of the disclosure comprise a therapeutically effective amount of at least one compound of the disclosure and a pharmaceutically acceptable carrier. Pharmaceutically acceptable carriers, which are useful, include, but are not limited to, glycerol, water, saline, ethanol, recombinant human albumin (e.g., RECOMBUMIN®), solubilized gelatins (e.g., GELOFUSINE®), and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), recombinant human albumin, solubilized gelatins, suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, isotonic agents, for example, sugars, sodium chloride, or polyalcohols such as mannitol and sorbitol, are included in the composition. Prolonged absorption of the injectable compositions may be brought about by including in the composition an agent that delays absorption, for example, aluminum monostearate or gelatin.

Formulations may be employed in admixtures with conventional excipients, i.e., pharmaceutically acceptable organic or inorganic carrier substances suitable for oral, parenteral, nasal, inhalational, intravenous, subcutaneous, transdermal enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring, and/or fragrance-conferring substances and the like. They may also be combined where desired with other active agents, e.g., other analgesic, anxiolytics or hypnotic agents. As used herein, “additional ingredients” include, but are not limited to, one or more ingredients that may be used as a pharmaceutical carrier.

The composition of the disclosure may comprise a preservative from about 0.005% to 2.0% by total weight of the composition. The preservative is used to prevent spoilage in the case of exposure to contaminants in the environment. Examples of preservatives useful in accordance with the disclosure include but are not limited to those selected from the group consisting of benzyl alcohol, sorbic acid, parabens, imidurea and any combinations thereof. One such preservative is a combination of about 0.5% to 2.0% benzyl alcohol and 0.05-0.5% sorbic acid.

The composition may include an antioxidant and a chelating agent that inhibit the degradation of the compound. Antioxidants for some compounds are BHT, BHA, alpha-tocopherol and ascorbic acid in the exemplary range of about 0.01% to 0.3%, or BHT in the range of 0.03% to 0.1% by weight by total weight of the composition. The chelating agent may be present in an amount of from 0.01% to 0.5% by weight by total weight of the composition. Exemplary chelating agents include edetate salts (e.g. disodium edetate) and citric acid in the weight range of about 0.01% to 0.20%, or in the range of 0.02% to 0.10% by weight by total weight of the composition. The chelating agent is useful for chelating metal ions in the composition that may be detrimental to the shelf life of the formulation. While BHT and disodium edetate are exemplary antioxidant and chelating agent, respectively, for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethyl cellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, acacia, and ionic or non-ionic surfactants. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the disclosure may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the disclosure may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, ionic and non-ionic surfactants, and a preservative. Additional excipients, such as fillers and sweetening, flavoring, or coloring agents, may also be included in these formulations.

A pharmaceutical composition of the disclosure may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying. Methods for mixing components include physical milling, the use of pellets in solid and suspension formulations and mixing in a transdermal patch, as known to those skilled in the art.

### Administration/Dosing

The regimen of administration may affect what constitutes an effective amount. The therapeutic formulations may be administered to the patient either prior to or after the onset of a disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present disclosure to a patient, such as a mammal, such as a human, may be carried out using known procedures, at dosages and for periods of time effective to treat, ameliorate, and/or prevent a disease or disorder contemplated herein. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the disclosure is from about 0.01 mg/kg to 100 mg/kg of body weight/per day. One of ordinary skill in the art would be able to study the relevant factors and make the determination regarding the effective amount of the therapeutic compound without undue experimentation.

The compound may be administered to an animal as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon a number of factors, such as, but not limited to, type and severity of the disease being treated, and type and age of the animal.

Actual dosage levels of the active ingredients in the pharmaceutical compositions of this disclosure may be varied so as to obtain an amount of the active ingredient that is effective to achieve the desired therapeutic response for a particular patient, composition, and mode of administration, without being toxic to the patient.

A medical doctor, e.g., physician or veterinarian, having ordinary skill in the art may readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the disclosure are dictated by and directly dependent on (a) the unique characteristics of the therapeutic compound and the particular therapeutic effect to be achieved, and (b) the limitations inherent in the art of compounding/formulating such a therapeutic compound for the treatment of a disease or disorder in a patient.

In certain embodiments, the compositions of the disclosure are administered to the patient in dosages that range from one to five times per day or more. In other embodiments, the compositions of the disclosure are administered to the patient in range of dosages that include, but are not limited to, once every day, every two days, every three days to once a week, and once every two weeks. It will be readily apparent to one skilled in the art that the frequency of administration of the various combination compositions of the disclosure will vary from subject to subject depending on many factors including, but not limited to, age, disease or disorder to be treated, gender, overall health, and other factors. Thus, the disclosure should not be construed to be limited to any particular dosage regime and the precise dosage and composition to be administered to any patient will be determined by the attending physician taking all other factors about the patient into account.

Compounds of the disclosure for administration may be in the range of from about 1 pg to about 7,500 mg, about 20 pg to about 7,000 mg, about 40 pg to about 6,500 mg, about 80 μg to about 6,000 mg, about 100 μg to about 5,500 mg, about 200 g g to about 5,000 mg, about 400 μg to about 4,000 mg, about 800 μg to about 3,000 mg, about 1 mg to about 2,500 mg, about 2 mg to about 2,000 mg, about 5 mg to about 1,000 mg, about 10 mg to about 750 mg, about 20 mg to about 600 mg, about 30 mg to about 500 mg, about 40 mg to about 400 mg, about 50 mg to about 300 mg, about 60 mg to about 250 mg, about 70 mg to about 200 mg, about 80 mg to about 150 mg, and any and all whole or partial increments there-in-between.

In some embodiments, the dose of a compound of the disclosure is from about 0.5 μg and about 5,000 mg. In some embodiments, a dose of a compound of the disclosure used in compositions described herein is less than about 5,000 mg, or less than about 4,000 mg, or less than about 3,000 mg, or less than about 2,000 mg, or less than about 1,000 mg, or less than about 800 mg, or less than about 600 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present disclosure is directed to a packaged pharmaceutical composition comprising a container holding a therapeutically effective amount of a compound of the disclosure, alone or in combination with a second pharmaceutical agent; and instructions for using the compound to treat, prevent, or reduce one or more symptoms of a disease or disorder in a patient.

The term “container” includes any receptacle for holding the pharmaceutical composition or for managing stability or water uptake. For example, in certain embodiments, the container is the packaging that contains the pharmaceutical composition, such as liquid (solution and suspension), semisolid, lyophilized solid, solution and powder or lyophilized formulation present in dual chambers. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, preventing, or reducing a disease or disorder in a patient.

### Administration

Routes of administration of any of the compositions of the disclosure include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, epidural, intrapleural, intraperitoneal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, emulsions, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

### Oral Administration

For oral application, particularly suitable are tablets, dragees, liquids, drops, capsules, caplets and gelcaps. Other formulations suitable for oral administration include, but are not limited to, a powdered or granular formulation, an aqueous or oily suspension, an aqueous or oily solution, a paste, a gel, toothpaste, a mouthwash, a coating, an oral rinse, or an emulsion. The compositions intended for oral use may be prepared according to any method known in the art and such compositions may contain one or more agents selected from the group consisting of inert, non-toxic, generally recognized as safe (GRAS) pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically controlled release tablets. Tablets may further comprise a sweetening agent, a flavoring agent, a coloring agent, a preservative, or some combination of these in order to provide for pharmaceutically elegant and palatable preparation. Hard capsules comprising the active ingredient may be made using a physiologically degradable composition, such as gelatin. The capsules comprise the active ingredient, and may further comprise additional ingredients including, for example, an inert solid diluent such as calcium carbonate, calcium phosphate, or kaolin.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin from animal-derived collagen or from a hypromellose, a modified form of cellulose, and manufactured using optional mixtures of gelatin, water and plasticizers such as sorbitol or glycerol. Such soft capsules comprise the active ingredient, which may be mixed with water or an oil medium such as peanut oil, liquid paraffin, or olive oil.

For oral administration, the compounds of the disclosure may be in the form of tablets or capsules prepared by conventional means with pharmaceutically acceptable excipients such as binding agents; fillers; lubricants; disintegrates; or wetting agents. If desired, the tablets may be coated using suitable methods and coating materials such as OPADRY® film coating systems available from Colorcon, West Point, Pa. (e.g., OPADRY® OY Type, OYC Type, Organic Enteric OY-P Type, Aqueous Enteric OY-A Type, OY-PM Type and OPADRY® White, 32K18400). It is understood that similar type of film coating or polymeric products from other companies may be used.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface-active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycolate. Known surface-active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e., having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e., drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) will melt.

The present disclosure also includes a multi-layer tablet comprising a layer providing for the delayed release of one or more compounds useful within the methods of the disclosure, and a further layer providing for the immediate release of one or more compounds useful within the methods of the disclosure. Using a wax/pH-sensitive polymer mix, a gastric insoluble composition may be obtained in which the active ingredient is entrapped, ensuring its delayed release.

Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl para-hydroxy benzoates or sorbic acid). Liquid formulations of a pharmaceutical composition of the disclosure which are suitable for oral administration may be prepared, packaged, and sold either in liquid form or in the form of a dry product intended for reconstitution with water or another suitable vehicle prior to use.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Injectable formulations may also be prepared, packaged, or sold in devices such as patient-controlled analgesia (PCA) devices. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3-butanediol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form in a recombinant human albumin, a fluidized gelatin, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### Topical Administration

An obstacle for topical administration of pharmaceuticals is the stratum corneum layer of the epidermis. The stratum corneum is a highly resistant layer comprised of protein, cholesterol, sphingolipids, free fatty acids and various other lipids, and includes cornified and living cells. One of the factors that limit the penetration rate (flux) of a compound through the stratum corneum is the amount of the active substance that can be loaded or applied onto the skin surface. The greater the amount of active substance which is applied per unit of area of the skin, the greater the concentration gradient between the skin surface and the lower layers of the skin, and in turn the greater the diffusion force of the active substance through the skin. Therefore, a formulation containing a greater concentration of the active substance is more likely to result in penetration of the active substance through the skin, and more of it, and at a more consistent rate, than a formulation having a lesser concentration, all other things being equal.

Formulations suitable for topical administration include, but are not limited to, liquid or semi-liquid preparations such as liniments, lotions, oil-in-water or water-in-oil emulsions such as creams, ointments or pastes, and solutions or suspensions. Topically administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

Enhancers of permeation may be used. These materials increase the rate of penetration of drugs across the skin. Typical enhancers in the art include ethanol, glycerol monolaurate, PGML (polyethylene glycol monolaurate), dimethylsulfoxide, and the like. Other enhancers include oleic acid, oleyl alcohol, ethoxydiglycol, laurocapram, alkanecarboxylic acids, dimethylsulfoxide, polar lipids, or N-methyl-2-pyrrolidone.

One acceptable vehicle for topical delivery of some of the compositions of the disclosure may contain liposomes. The composition of the liposomes and their use are known in the art (i.e., U.S. Pat. No. 6,323,219).

In alternative embodiments, the topically active pharmaceutical composition may be optionally combined with other ingredients such as adjuvants, anti-oxidants, chelating agents, surfactants, foaming agents, wetting agents, emulsifying agents, viscosifiers, buffering agents, preservatives, and the like. In other embodiments, a permeation or penetration enhancer is included in the composition and is effective in improving the percutaneous penetration of the active ingredient into and through the stratum corneum with respect to a composition lacking the permeation enhancer. Various permeation enhancers, including oleic acid, oleyl alcohol, ethoxydiglycol, laurocapram, alkanecarboxylic acids, dimethylsulfoxide, polar lipids, or N-methyl-2-pyrrolidone, are known to those of skill in the art. In another aspect, the composition may further comprise a hydrotropic agent, which functions to increase disorder in the structure of the stratum corneum, and thus allows increased transport across the stratum corneum. Various hydrotropic agents such as isopropyl alcohol, propylene glycol, or sodium xylene sulfonate, are known to those of skill in the art.

The topically active pharmaceutical composition should be applied in an amount effective to affect desired changes. As used herein “amount effective” shall mean an amount sufficient to cover the region of skin surface where a change is desired. An active compound should be present in the amount of from about 0.0001% to about 15% by weight volume of the composition. For example, it should be present in an amount from about 0.0005% to about 5% of the composition; for example, it should be present in an amount of from about 0.001% to about 1% of the composition. Such compounds may be synthetically- or naturally derived.

### Buccal Administration

A pharmaceutical composition of the disclosure may be prepared, packaged, or sold in a formulation suitable for buccal administration. Such formulations may, for example, be in the form of tablets or lozenges made using conventional methods, and may contain, for example, 0.1 to 20% (w/w) of the active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, may have an average particle or droplet size in the range from about 0.1 to about 200 nanometers, and may further comprise one or more of the additional ingredients described herein. The examples of formulations described herein are not exhaustive and it is understood that the disclosure includes additional modifications of these and other formulations not described herein, but which are known to those of skill in the art.

### Rectal Administration

A pharmaceutical composition of the disclosure may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants, and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants, and preservatives.

### Additional Administration Forms

Additional dosage forms of this disclosure include dosage forms as described in U.S. Pat. Nos. 6,340,475, 6,488,962, 6,451,808, 5,972,389, 5,582,837, and 5,007,790. Additional dosage forms of this disclosure also include dosage forms as described in U.S. Patent Applications Nos. 20030147952, 20030104062, 20030104053, 20030044466, 20030039688, and 20020051820. Additional dosage forms of this disclosure also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Controlled Release Formulations and Drug Delivery Systems:

In certain embodiments, the compositions and/or formulations of the present disclosure may be, but are not limited to, short-term, rapid-onset and/or rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the disclosure may be administered in the form of microparticles, for example, by injection or in the form of wafers or discs by implantation.

In certain embodiments of the disclosure, the compounds useful within the disclosure are administered to a subject, alone or in combination with another pharmaceutical agent, using a sustained release formulation.

The term delayed release is used herein in its conventional sense to refer to a drug formulation that provides for an initial release of the drug after some delay following drug administration and that may, although not necessarily, include a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Those skilled in the art will recognize or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that, wherever values and ranges are provided herein, the description in range format is merely for convenience and brevity and should not be construed as an inflexible limitation on the scope of the disclosure. Accordingly, all values and ranges encompassed by these values and ranges are meant to be encompassed within the scope of the present disclosure. Moreover, all values that fall within these ranges, as well as the upper or lower limits of a range of values, are also contemplated by the present application. The description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The following examples further illustrate aspects of the present disclosure. However, they are in no way a limitation of the teachings or disclosure of the present disclosure as set forth herein.

### EXAMPLES

The disclosure is now described with reference to the following Examples. These Examples are provided for the purpose of illustration only, and the disclosure is not limited to these Examples, but rather encompasses all variations that are evident as a result of the teachings provided herein.

### Materials & Methods

The following procedures can be utilized in evaluating and selecting compounds that inhibit hepatitis B virus infection.

HepDE19 assay with bDNA quantitation of HBV rcDNA:

HepDE19 cell culture system is a HepG2 (human hepatocarcinoma) derived cell line that supports HBV DNA replication and cccDNA formation in a tetracycline (Tet)-regulated manner and produces HBV rcDNA and a detectable reporter molecule dependent on the production and maintenance of cccDNA (Guo, et al., 2007, J. Virol. 81:12472-12484).

HepDE19 (50,000 cells/well) were plated in 96-well collagen-coated tissue-culture treated microtiter plates in DMEM/F12 medium supplemented with 10% fetal bovine serum, 1% penicillin-streptomycin and 1 μg/mL tetracycline and incubated in a humidified incubator at 37° C. and 5% CO₂ overnight. Next day, the cells were switched to fresh medium without tetracycline and incubated for 4 hours at 37° C. and 5% CO₂. The cells were treated with fresh Tet-free medium with compounds at concentrations starting at 25 μM and a serial, ½ log, 8-point, titration series in duplicate. The final DMSO concentration in the assay was 0.5%. The plates were incubated for 7 days in a humidified incubator at 37° C. and 5% CO₂. Following a 7 day-incubation, the level of rcDNA present in the inhibitor-treated wells was measured using a Quantigene 2.0 bDNA assay kit (Affymetrix, Santa Clara, CA) with HBV specific custom probe set and manufacturers instructions. Concurrently, the effect of compounds on cell viability was assessed using replicate plates, plated at a density of 5,000 cells/well and incubated for 4 days, to determine the ATP content as a measure of cell viability using the cell-titer glo reagent (CTG; Promega Corporation, Madison, WI) as per manufacturer's instructions. The plates were read using a Victor luminescence plate reader (PerkinElmer Model 1420 Multilabel counter) and the relative luminescence units (RLU) data generated from each well was calculated as % inhibition of the untreated control wells and analyzed using XL-Fit module in Microsoft Excel to determine EC₅₀ and EC₉₀ (bDNA) and CC₅₀ (CTG) values using a 4-parameter curve fitting algorithm.

### LCMS Methods:

LCMS Method A: Waters Acquity UPLC system employing a Waters Acquity UPLC BEH C18, 1.7 μm), 50×2.1 mm column with an aqueous acetonitrile based solvent gradient of 2-98% CH₃CN/H₂O (0.05% TFA) over 9.5 mins. Flow rate=0.8 mL/min.
LCMS Method B: Shimadzu UFLC system employing an ACE UltraCore Super PhenylHexyl, 2.5 μm), 50×2.1 mm column with an aqueous acetonitrile based solvent gradient of 5-100% CH₃CN/H₂O (0.05% Formic acid) over 4.0 mins followed by maintenance of 100% CH₃CN (0.05% Formic acid) for a further 1 min. Flow rate=1.0 mL/min.
LCMS Method C: Waters Acquity UPLC system employing a Waters Acquity C18 1.7 μm, 2.1×50 mm column, Mobile phase-A: 0.1% formic acid in H₂O. Mobile phase-B: CH₃CN, Solvent gradient: 0.0-0.3 min—isocratic 5% B, 0.3-2.5 min—linear gradient of 5-95% B, 2.5-3.7 min—isocratic 95% B, 3.7-4.0 min—linear gradient of 95-5% B, 4.0-4.6 min—isocratic 5% B. Flow rate=0.5 mL/min, Temp: 40° C.;
LCMS Method D: Waters Acquity UPLC system employing a Waters Acquity UPLC BEH C_{18 1.7} μm, 2.1×50 mm, Mobile phase-A: 10 mM Ammonium Bicarbonate in water, Mobile phase-B: CH₃CN, Solvent gradient: 0.0-0.3 min—isocratic 5% B, 0.3-2.5 min—linear gradient of 5-95% B, 2.5-3.7 min—isocratic 95% B, 3.7-4.0 min—linear gradient of 95-5% B, 4.0-4.6 min—isocratic 5% B., Flow rate=0.5 mL/min, Temp: 40° C.
LCMS Method E: Waters Acquity UPLC system employing a Waters Acquity UPLC BEH C18 1.7 μm, 2.1×50 mm, Mobile phase-A: 0.1% formic acid in H₂O. Mobile phase-B: MeCN, Solvent gradient: % B: 0.0-0.3 min—isocratic 5% B, 0.3-2.5 min—linear gradient of 5-95% B, 2.5-3.7 min—isocratic 95% B, 3.7-4.0 min—linear gradient of 95-5% B, 4.0-4.6 min—isocratic 5% B, Flow rate=0.6 mL/min, Temp: 40° C.

As described herein, “Enantiomer I” or “Diastereomer I” or “Stereoisomer I” refers to the first enantiomer or diastereomer or stereoisomer eluded from the chiral column under the specific chiral analytical conditions detailed for examples provided elsewhere herein; and “Enantiomer II” or “Diastereomer II” or “Stereoisomer II” refers to the second enantiomer or diastereomer or stereoisomer eluded from the chiral column under the specific chiral analytical conditions detailed for examples provided elsewhere herein. Such nomenclature does not imply or impart any particular relative and/or absolute configuration for these compounds.

### Synthesis of 6,7-difluoroisoquinolin-1(2H)-one (IIa)

### (E)-3-(3,4-Difluorophenyl)acryloyl azide

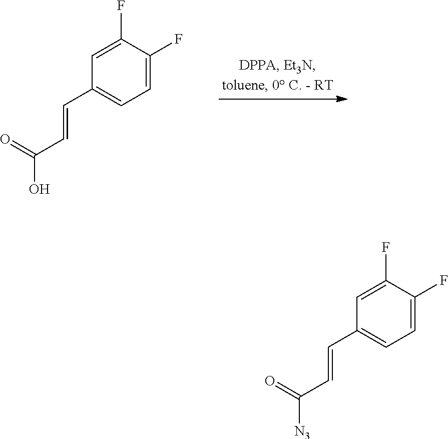 [SMILES: O=C(O)/C=C/c1ccc(F)c(F)c1 ; [N-]=[N+]=NC(=O)/C=C/c1ccc(F)c(F)c1]

To a solution of 20.0 g (108.1 mmol, 1.0 eq.) of (E)-3-(3,4-difluorophenyl)acrylic acid in 100 mL of toluene under a nitrogen atmosphere at 0° C. was added 45 mL (324.1 mmol, 3.0 eq.) of triethylamine followed by 26.8 g (97.8 mmol, 0.9 eq.) of diphenylphosphoryl azide. The mixture was allowed to warm to room temperature and stirred for 2 h. The solvent was removed in vacuo and the product was isolated by MPLC (REVELERIS® silica column; eluting with a linear gradient of 10-20% ethyl acetate/petroleum ether) to provide 10.0 g (47.84 mmol, 44% yield) of (E)-3-(3,4-difluorophenyl)acryloyl azide. ¹H NMR (400 MHz, CDCl₃): δ 7.65 (d, 1H), 7.33-7.39 (m, 1H), 7.25-7.30 (m, 1H), 7.19-7.23 (m, 1H), 6.34 (d, 1H).

### 6,7-Difluoroisoquinolin-1(2H)-one (IIa)

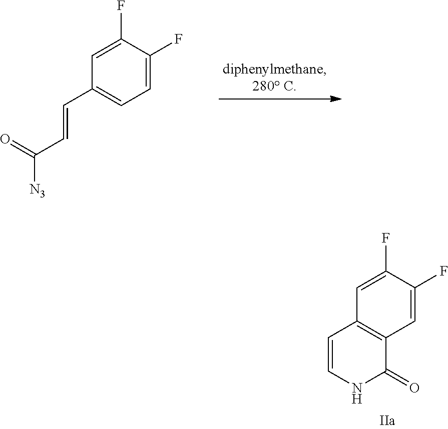 [SMILES: [N-]=[N+]=NC(=O)/C=C/c1ccc(F)c(F)c1 ; O=c1[nH]ccc2cc(F)c(F)cc12]

A stirred solution of 10.0 g (47.8 mmol, 1.0 eq.) of (E)-3-(3,4-difluorophenyl)acryloyl azide in 50 mL of diphenylmethane was heated to 100° C. for 30 min. The temperature was subsequently increased to 280° C. and stirring was continued for 3 h. The mixture was allowed to cool to room temperature and diluted with 200 mL of n-heptane and stirred for a further 30 min. The solids were collected by filtration and triturated with 100 mL of n-heptane and dried under vacuum to provide 6.0 g (33.1 mmol, 69% yield) of 6,7-difluoroisoquinolin-1(2H)-one (IIa). LCMS: m/z found 182.4 [M+H]⁺, RT=1.45 min; ¹H NMR (400 MHz, CDCl₃): δ 10.11 (bs, 1H), 8.15-8.21 (m, 1H), 7.30-7.35 (m, 1H), 7.11-7.14 (m, 1H), 6.48 (d, 1H).

### 4-Bromo-6,7-difluoroisoquinolin-1(2H)-one (IIIa)

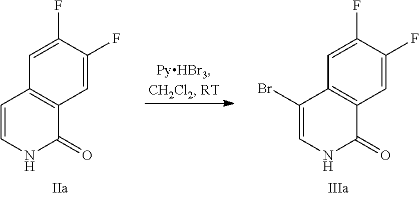 [SMILES: O=c1[nH]ccc2cc(F)c(F)cc12 ; O=c1[nH]cc(Br)c2cc(F)c(F)cc12]

To a solution of 3.0 g (16.6 mmol, 1.0 eq.) of 6,7-difluoroisoquinolin-1(2H)-one (IIa) in 30 mL of methylene chloride was added 5.3 g (16.6 mmol, 1.0 eq.) of pyridinium hydrobromide perbromide and the mixture was stirred at room temperature for 4 h. The reaction was quenched with 50 mL of saturated sodium bicarbonate solution and the solvent was removed in vacuo. The residue was suspended in 80 mL of water and the solids were collected by filtration, washed with 50 mL of petroleum ether and dried under vacuum to provide 3.5 g (13.5 mmol, 81% yield) of 4-bromo-6,7-difluoroisoquinolin-1(2H)-one (IIIa). ¹H NMR (300 MHz, DMSO-d₆): δ 11.81 (bs, 1H), 8.11-8.18 (m, 1H), 7.68-7.75 (m, 1H), 7.64 (s, 1H).

### 4-Acetyl-6,7-difluoroisoquinolin-1(2H)-one (XXa)

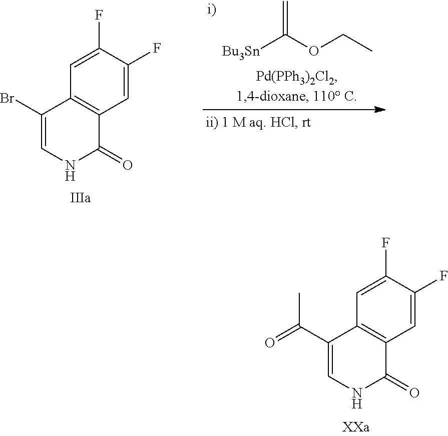

To a stirred solution of 3.5 g (13.5 mmol, 1.0 eq.) of 4-bromo-6,7-difluoroisoquinolin-1(2H)-one (IIIa) in 35 mL of 1,4-dioxane was added 12.2 g (33.8 mmol, 2.5 eq.) of tributyl(1-ethoxyvinyl)stannane. The mixture was purged with nitrogen gas for 5 min and 0.95 g (1.35 mmol, 0.1 eq.) of Pd(PPh₃)₂Cl₂ was added, and then heated to 110° C. for 16 h. The reaction mixture was allowed to cool to room temperature and 60 mL of 1 M aqueous HCl was added and stirring was continued for an additional 1 h. The reaction mixture was then basified with 50 mL of saturated sodium bicarbonate solution and extracted with ethyl acetate (3×200 mL). The combined organic extracts were washed with 100 mL of water, 100 mL of brine, dried (Na₂SO₄), filtered and the solvent was removed in vacuo. The residue was purified by MPLC (REVELERIS® silica column, eluting with a linear gradient of 30-50% ethyl acetate/petroleum ether) to provide 1.7 g (7.6 mmol, 56% yield) of 4-acetyl-6,7-difluoroisoquinolin-1(2H)-one (XXa). LCMS: m/z found 224.0 [M+H]⁺, ¹H NMR (300 MHz, DMSO-d₆): δ 12.20 (bs, 1H), 8.87-8.95 (m, 1H), 8.27 (s, 1H), 8.09-8.16 (m, 1H), 2.53 (s, 3H).

The above reaction sequence was performed on multiple batches with consistent results.

### General Procedure I, Reductive Alkylation.

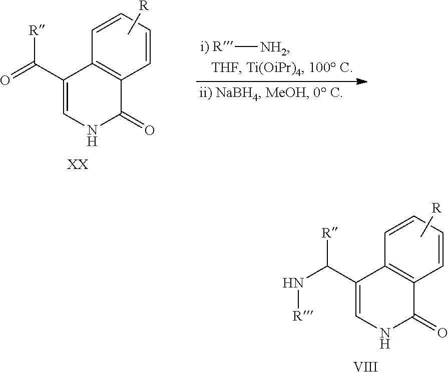

To a solution of 1.0 eq. of ketone XX in anhydrous THE was added 4.4 eq. of a primary amine followed by 7.0 eq. of titanium isopropoxide and the mixture was heated in a sealed tube at 100° C. for 16 h. The mixture was allowed to cool to room temperature and further cooled to 0° C. Following dilution with methanol, 3.0 eq. of sodium borohydride was added portion-wise over approximately 10 min and stirring was continued for 4 h. The reaction mixture was diluted with water and extracted four times with ethyl acetate. The combined organic extracts were washed with water, brine, dried (Na₂SO₄), filtered and the solvent was removed in vacuo to provide secondary amine VIII.

### 6,7-Difluoro-4-(1-(methylamino)ethyl)isoquinolin-1(2H)-one (VIIIa)

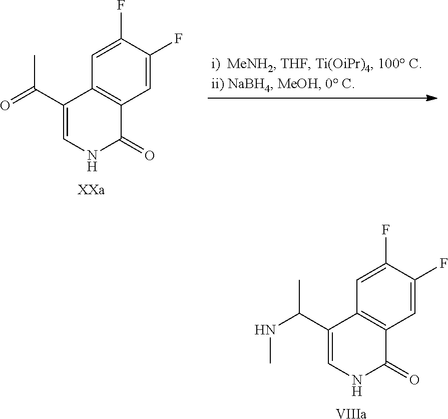

6,7-Difluoro-4-(1-(methylamino)ethyl)isoquinolin-1(2H)-one (VIIIa) was synthesized according to General Procedure I from 4-acetyl-6,7-difluoroisoquinolin-1(2H)-one (XXa) and a 2.0 M solution of methyl amine in THF. LCMS: m/z found 239.0 [M+H]⁺.

### 6,7-Difluoro-4-(1-(ethylamino)ethyl)isoquinolin-1(2H)-one (VIIIb)

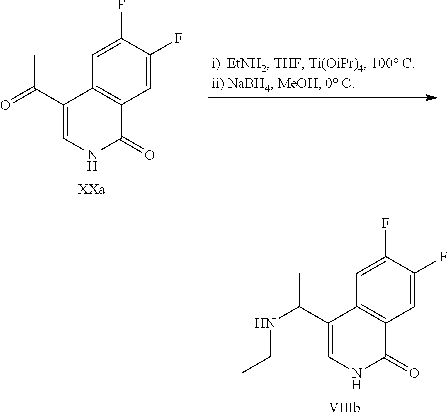

6,7-Difluoro-4-(1-(ethylamino)ethyl)isoquinolin-1(21)-one (VIIIb) was synthesized according to General Procedure I from 4-acetyl-6,7-difluoroisoquinolin-1(2H)-one (XXa) and a 2.0 M solution of ethyl amine in THF. LCMS: m/z found 253.0 [M+H]⁺.

### 6,7-Difluoro-4-(1-(isobutylamino)ethyl)isoquinolin-1(2H)-one (VIIIc)

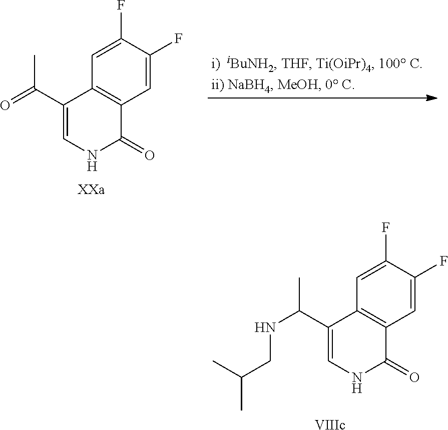

6,7-Difluoro-4-(1-(isobutylamino)ethyl)isoquinolin-1(2H)-one (VIIIc) was synthesized according to General Procedure I from 4-acetyl-6,7-difluoroisoquinolin-1(2H)-one (XXa) and isobutylamine. LCMS: m/z found 281.1 [M+H]⁺.

### 6,7-Difluoro-4-(1-((3-hydroxypropyl)amino)ethyl)isoquinolin-1(2H)-one (VIIId)

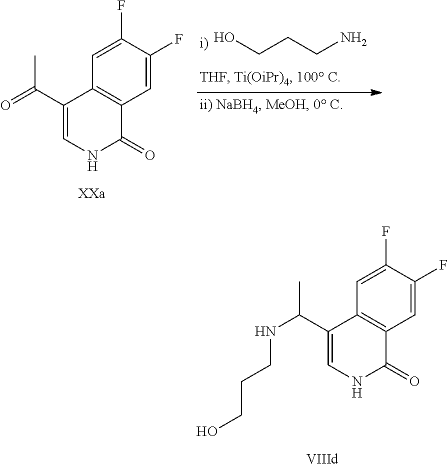 [SMILES: CC(=O)c1c[nH]c(=O)c2cc(F)c(F)cc12 ; NCCCO ; CC(NCCCO)c1c[nH]c(=O)c2cc(F)c(F)cc12]

6,7-Difluoro-4-(1-(isobutylamino)ethyl)isoquinolin-1(2H)-one (VIIId) was synthesized according to General Procedure I from 4-acetyl-6,7-difluoroisoquinolin-1(2H)-one (XXa) and 3-hydroxypropylamine. LCMS: m/z found 283.1 [M+H]⁺.

### 4-(1-((3-((tert-Butyldimethylsilyl)oxy)propyl)amino)ethyl)-6,7-difluoroisoquinolin-1(2H)-one (VIIIe)

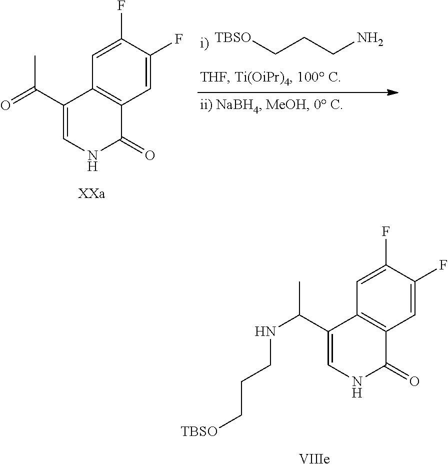 [SMILES: CC(=O)c1c[nH]c(=O)c2cc(F)c(F)cc12 ; CC(C)(C)[Si](C)(C)OCCCN ; CC(NCCCO[Si](C)(C)C(C)(C)C)c1c[nH]c(=O)c2cc(F)c(F)cc12]

4-(1-((3-((tert-Butyldimethylsilyl)oxy)propyl)amino)ethyl)-6,7-difluoroisoquinolin-1(2H)-one (VIIIe) was synthesized according to General Procedure I from 4-acetyl-6,7-difluoroisoquinolin-1(2H)-one (XXa) and 3-((tert-butyldimethylsilyl)oxy)propan-1-amine. LCMS: m/z found 397.4 [M+H]⁺. ¹H NMR (400 MHz, CDCl₃): δ 10.24 (bs, 1H), 8.22 (dd, 1H) 7.90 (dd, 1H), 7.24 (s, 1H), 3.97 (q, 1H), 3.71-3.65 (m, 2H), 2.71-2.60 (m, 2H), 1.68 (q, 2H), 1.41 (d, 3H), 0.85 (s, 9H), 0.02 (s, 6H).

### N-(1-(6,7-Difluoro-1-oxo-1,2-dihydroisoquinolin-4-yl)ethyl)-N-methyl-1H-indole-2-carboxamide (Compounds 1 & 2)

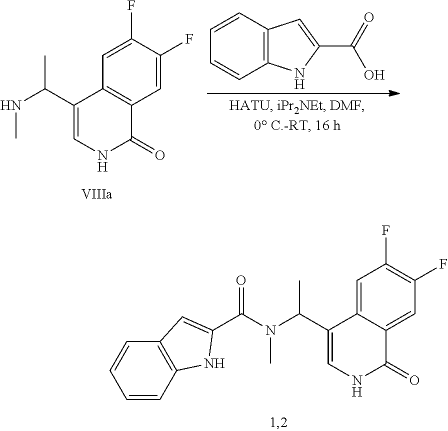

To a stirred solution of 50 mg (0.31 mmol, 1.0 eq.) of 1H-indole-2-carboxylic acid in 1 mL of DMF was added 0.13 mL (0.77 mmol, 2.5 eq.) of N,N-diisopropylethylamine followed by 0.14 g (0.37 mmol, 1.2 eq.) of HATU at 0° C. and the mixture was stirred for 20 min. A solution of 89 mg (0.37 mmol, 1.2 eq.) of 1H-6,7-difluoro-4-(1-(methylamino)ethyl)isoquinolin-1(2H)-one (VIIIa) in 1 mL of DMF was added at 0° C. and the mixture was stirred at room temperature for 16 h. The mixture was then poured into 20 mL of ice-water and stirred for 10 min. The precipitated solid was collected by filtration and dried under vacuum to provide 70 mg (0.18 mmol, 59% yield) of racemic N-(1-(6,7-difluoro-1-oxo-1,2-dihydroisoquinolin-4-yl)ethyl)-N-methyl-1H-indole-2-carboxamide. LCMS: m/z found 382.2 [M+H]⁺. The enantiomers were subsequently separated by chiral SFC, Column: Chiralpak IC (250×30 mm, 5 μm) 60% CO₂/MeOH, Flow rate 100 g/min.

N-(1-(6,7-Difluoro-1-oxo-1,2-dihydroisoquinolin-4-yl)ethyl)-N-methyl-1H-indole-2-carboxamide—Enantiomer I (Compound 1), LCMS: m/z found 382.3 [M+H]⁺, RT=3.96 min (Method A); ¹H NMR (400 MHz, DMSO-d₆): δ 11.68 (bs, 2H), 8.13-8.08 (m, 1H), 7.61-7.57 (m, 2H), 7.46 (d, 1H), 7.36 (s, 1H), 7.22-7.18 (m, 1H), 7.05-7.01 (m, 1H), 6.88 (s, 1H), 6.14-6.08 (m, 1H), 2.93 (s, 3H), 1.54 (d, 3H); Chiral analytical SFC: RT=3.06 min, Column: Chiralpak IC, (4.6×150 mm, 3 μm), 60% CO₂/MeOH, Flow=3.0 g/min.

N-(1-(6,7-Difluoro-1-oxo-1,2-dihydroisoquinolin-4-yl)ethyl)-N-methyl-1H-indole-2-carboxamide—Enantiomer II (Compound 2), LCMS: m/z found 382.3 [M+H]⁺, RT=3.96 min (Method A); ¹H NMR (400 MHz, DMSO-d₆): δ 11.68 (bs, 2H), 8.13-8.08 (m, 1H), 7.61-7.57 (m, 2H), 7.46 (d, 1H), 7.36 (s, 1H), 7.22-7.18 (m, 1H), 7.05-7.01 (m, 1H), 6.88 (s, 1H), 6.14-6.08 (m, 1H), 2.93 (s, 3H), 1.54 (d, 3H); Chiral analytical SFC: RT=4.94 min, Column: Chiralpak IC, (4.6×150 mm, 3 μm), 60% CO₂/MeOH, Flow=3.0 g/min.

### N-(1-(6,7-Difluoro-1-oxo-1,2-dihydroisoquinolin-4-yl)ethyl)-N-isobutyl-1H-indole-2-carboxamide (Compounds 3 & 4)

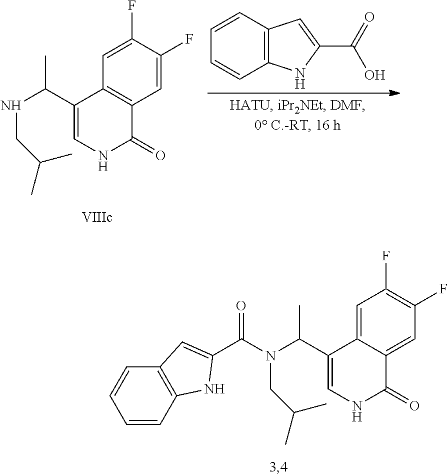

Racemic N-(1-(6,7-difluoro-1-oxo-1,2-dihydroisoquinolin-4-yl)ethyl)-N-isobutyl-1H-indole-2-carboxamide was synthesized in a similar manner as described above from 6,7-difluoro-4-(1-(isobutylamino)ethyl)isoquinolin-1(2H)-one (VIIIc) and 1H-indole-2-carboxylic acid. The enantiomers were subsequently separated by chiral SFC, Column: Lux Cellulose-2 (250×30 mm, 5 μm) 50% CO₂/MeOH, Flow rate 70 g/min.

N-(1-(6,7-Difluoro-1-oxo-1,2-dihydroisoquinolin-4-yl)ethyl)-N-isobutyl-1H-indole-2-carboxamide—Enantiomer I (Compound 3), LCMS: m/z found 424.3 [M+H]⁺, RT=4.62 min (Method A); ¹H NMR (400 MHz, DMSO-d₆): δ 11.71 (bs, 2H), 8.14-8.10 (m, 1H), 7.63-7.59 (m, 2H), 7.46-7.42 (m, 2H), 7.21-7.17 (m, 1H), 7.06-7.02 (m, 1H), 6.86-6.81 (m, 1H), 6.09-6.04 (m, 1H), 3.25-3.19 (m, 2H), 1.64 (d, 3H), 1.51-1.48 (m, 1H), 0.58 (d, 3H), 0.43 (d, 3H); Chiral analytical SFC: RT=1.40 min, Column: Chiralcel OZ-3, (4.6×150 mm, 3 m), 60% CO₂/MeOH, Flow=3.0 g/min.

N-(1-(6,7-Difluoro-1-oxo-1,2-dihydroisoquinolin-4-yl)ethyl)-N-isobutyl-1H-indole-2-carboxamide—Enantiomer II (Compound 4), LCMS: m/z found 424.3 [M+H]⁺, RT=4.62 min (Method A); ¹H NMR (400 MHz, DMSO-d₆): δ 11.71 (bs, 2H), 8.14-8.10 (m, 1H), 7.63-7.59 (m, 2H), 7.46-7.42 (m, 2H), 7.21-7.17 (m, 1H), 7.06-7.02 (m, 1H), 6.86-6.81 (m, 1H), 6.09-6.04 (m, 1H), 3.25-3.19 (m, 2H), 1.64 (d, 3H), 1.51-1.48 (m, 1H), 0.58 (d, 3H), 0.43 (d, 3H); Chiral analytical SFC: RT=2.33 min, Column: Chiralcel OZ-3, (4.6×150 mm, 3 μm), 60% CO₂/MeOH, Flow=3.0 g/min.

### N-(1-(6,7-Difluoro-1-oxo-1,2-dihydroisoquinolin-4-yl)ethyl)-N-ethyl-1H-indole-2-carboxamide (Compounds 5 & 6)

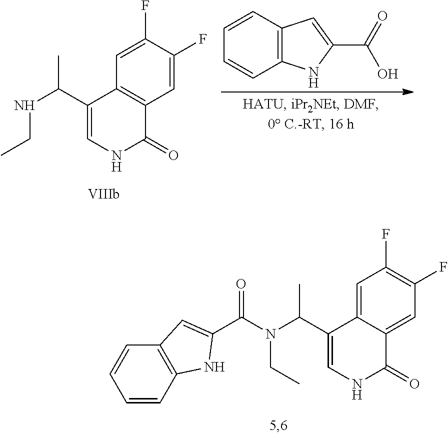

Racemic N-(1-(6,7-difluoro-1-oxo-1,2-dihydroisoquinolin-4-yl)ethyl)-N-ethyl-1H-indole-2-carboxamide was synthesized in a similar manner as described above from 6,7-difluoro-4-(1-(ethylamino)ethyl)isoquinolin-1(2H)-one (VIIIb) and 1H-indole-2-carboxylic acid. The enantiomers were subsequently separated by chiral SFC, Column: Chiralpak IC (250×30 mm, 5 μm) 65% CO₂/MeOH, Flow rate 100 g/min.

N-(1-(6,7-Difluoro-1-oxo-1,2-dihydroisoquinolin-4-yl)ethyl)-N-ethyl-1H-indole-2-carboxamide—Enantiomer I (Compound 5), LCMS: m/z found 396.3 [M+H]⁺, RT=4.18 min (Method A); ¹H NMR (400 MHz, DMSO-d₆): δ 11.71 (bs, 2H), 8.15-8.10 (m, 1H), 7.65-7.46 (m, 3H), 7.42 (s, 1H), 7.22-7.18 (m, 1H), 7.06-7.02 (m, 1H), 6.88 (s, 1H), 6.17-6.13 (m, 1H), 3.55-3.46 (m, 2H), 1.57 (bd, 3H), 0.76 (t, 3H); Chiral analytical SFC: RT=3.37 min, Column: Chiralpak IC, (4.6×150 mm, 3 μm), 60% CO₂/MeOH, Flow=4.0 g/min.

N-(1-(6,7-Difluoro-1-oxo-1,2-dihydroisoquinolin-4-yl)ethyl)-N-ethyl-1H-indole-2-carboxamide—Enantiomer II (Compound 6), LCMS: m/z found 396.3 [M+H]⁺, RT=4.18 min (Method A); ¹H NMR (400 MHz, DMSO-d₆): δ 11.71 (bs, 2H), 8.15-8.10 (m, 1H), 7.65-7.46 (m, 3H), 7.42 (s, 1H), 7.22-7.18 (m, 1H), 7.06-7.02 (m, 1H), 6.88 (s, 1H), 6.17-6.13 (m, 1H), 3.55-3.46 (m, 2H), 1.57 (bd, 3H), 0.76 (t, 3H); Chiral analytical SFC: RT=6.56 min, Column: Chiralpak IC, (4.6×150 mm, 3 μm), 60% CO₂/MeOH, Flow=4.0 g/min.

### N-(1-(6,7-Difluoro-1-oxo-1,2-dihydroisoquinolin-4-yl)ethyl)-4-fluoro-N-methyl-1H-indole-2-carboxamide (Compounds 7 & 8)

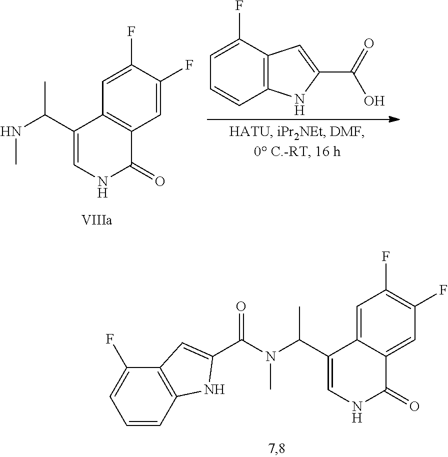

Racemic N-(1-(6,7-difluoro-1-oxo-1,2-dihydroisoquinolin-4-yl)ethyl)-4-fluoro-N-methyl-1H-indole-2-carboxamide was synthesized in a similar manner as described above from 1H-6,7-difluoro-4-(1-(methylamino)ethyl)isoquinolin-1(2H)-one (VIIIa) and 4-fluoro-1H-indole-2-carboxylic acid. The enantiomers were subsequently separated by chiral SFC, Column: Chiralpak IC (250×30 mm, 5 μm) 60% CO₂/MeOH, Flow rate 90 g/min.

N-(1-(6,7-Difluoro-1-oxo-1,2-dihydroisoquinolin-4-yl)ethyl)-4-fluoro-N-methyl-1H-indole-2-carboxamide—Enantiomer I (Compound 7), LCMS: m/z found 400.3 [M+H]⁺, RT=4.14 min (Method A); ¹H NMR (400 MHz, DMSO-d₆): δ 11.89 (bs, 2H), 8.15-8.10 (m, 1H), 7.63-7.60 (m, 1H), 7.35-7.29 (m, 2H), 7.21-7.15 (m, 1H), 6.92 (s, 1H), 6.83-6.78 (m, 1H), 6.13-6.10 (m, 1H), 2.95 (s, 3H), 1.54 (d, 3H); Chiral analytical SFC: RT=3.16 min, Column: Chiralpak IC, (4.6×150 mm, 3 μm), 70% CO₂/MeOH, Flow=3.0 g/min.

N-(1-(6,7-Difluoro-1-oxo-1,2-dihydroisoquinolin-4-yl)ethyl)-4-fluoro-N-methyl-1H-indole-2-carboxamide—Enantiomer II (Compound 8), LCMS: m/z found 400.3 [M+H]⁺, RT=4.14 min (Method A); ¹H NMR (400 MHz, DMSO-d₆): δ 11.89 (bs, 2H), 8.15-8.10 (m, 1H), 7.63-7.60 (m, 1H), 7.35-7.29 (m, 2H), 7.21-7.15 (m, 1H), 6.92 (s, 1H), 6.83-6.78 (m, 1H), 6.13-6.10 (m, 1H), 2.95 (s, 3H), 1.54 (d, 3H); Chiral analytical SFC: RT=4.55 min, Column: Chiralpak IC, (4.6×150 mm, 3 μm), 70% CO₂/MeOH, Flow=3.0 g/min.

### N-(1-(6,7-Difluoro-1-oxo-1,2-dihydroisoquinolin-4-yl)ethyl)-5-fluoro-N-methyl-1H-indole-2-carboxamide (Compounds 9 & 10)

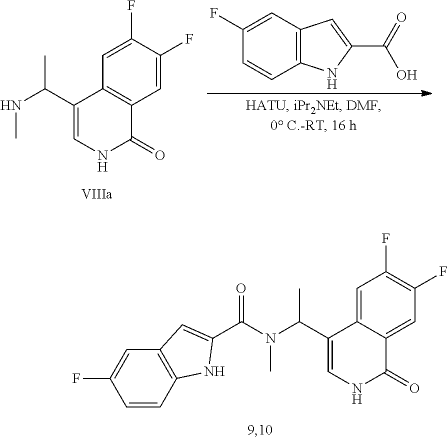

Racemic N-(1-(6,7-difluoro-1-oxo-1,2-dihydroisoquinolin-4-yl)ethyl)-5-fluoro-N-methyl-1H-indole-2-carboxamide was synthesized in a similar manner as described above from 1H-6,7-difluoro-4-(1-(methylamino)ethyl)isoquinolin-1(2H)-one (VIIIa) and 5-fluoro-1H-indole-2-carboxylic acid. The enantiomers were subsequently separated by chiral SFC, Column: Chiralpak IC (250×30 mm, 5 μm) 80% CO₂/MeOH, Flow rate 100 g/min.

N-(1-(6,7-Difluoro-1-oxo-1,2-dihydroisoquinolin-4-yl)ethyl)-5-fluoro-N-methyl-1H-indole-2-carboxamide—Enantiomer I (Compound 9), LCMS: m/z found 400.2 [M+H]⁺, RT=4.08 min (Method A); ¹H NMR (400 MHz, DMSO-d₆): δ 11.81 (bs, 1H), 11.71 (bs, 1H), 8.15-8.10 (m, 1H), 7.64-7.59 (m, 1H), 7.47-7.44 (m, 1H), 7.37-7.29 (m, 2H), 7.09-7.04 (m, 1H), 6.86 (s, 1H), 6.14-6.08 (m, 1H), 2.92 (s, 3H), 1.54 (d, 3H); Chiral analytical SFC: RT=3.45 min, Column: Chiralpak IC-3, (4.6×150 mm, 3 μm), 70% CO₂/MeOH, Flow=3.0 g/min.

N-(1-(6,7-Difluoro-1-oxo-1,2-dihydroisoquinolin-4-yl)ethyl)-5-fluoro-N-methyl-1H-indole-2-carboxamide—Enantiomer II (Compound 10), LCMS: m/z found 400.2 [M+H]⁺, RT=4.08 min (Method A); ¹H NMR (400 MHz, DMSO-d₆): δ 11.81 (bs, 1H), 11.71 (bs, 1H), 8.15-8.10 (m, 1H), 7.64-7.59 (m, 1H), 7.47-7.44 (m, 1H), 7.37-7.29 (m, 2H), 7.09-7.04 (m, 1H), 6.86 (s, 1H), 6.14-6.08 (m, 1H), 2.92 (s, 3H), 1.54 (d, 3H); Chiral analytical SFC: RT=5.26 min, Column: Chiralpak IC-3, (4.6×150 mm, 3 μm), 70% CO₂/MeOH, Flow=3.0 g/min.

### N-(1-(6,7-Difluoro-1-oxo-1,2-dihydroisoquinolin-4-yl)ethyl)-6-fluoro-N-methyl-1H-indole-2-carboxamide (Compounds 11 & 12)

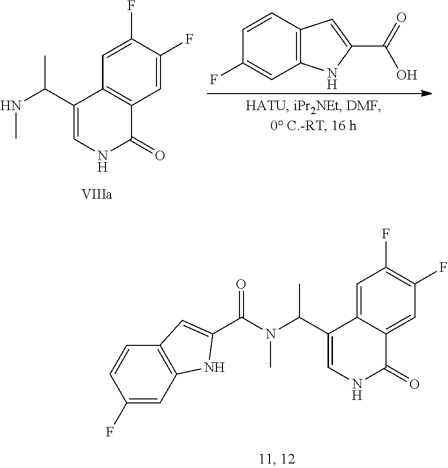 [SMILES: CNC(C)c1c[nH]c(=O)c2cc(F)c(F)cc12 ; O=C(O)c1cc2ccc(F)cc2[nH]1 ; CC(c1c[nH]c(=O)c2cc(F)c(F)cc12)N(C)C(=O)c1cc2ccc(F)cc2[nH]1]

Racemic N-(1-(6,7-difluoro-1-oxo-1,2-dihydroisoquinolin-4-yl)ethyl)-6-fluoro-N-methyl-1H-indole-2-carboxamide was synthesized in a similar manner as described above from 1H-6,7-difluoro-4-(1-(methylamino)ethyl)isoquinolin-1(2H)-one (VIIIa) and 6-fluoro-1H-indole-2-carboxylic acid. The enantiomers were subsequently separated by chiral SFC, Column: Chiralpak IC (250×30 mm, 5 μm) 65% CO₂/MeOH, Flow rate 100 g/min.

N-(1-(6,7-Difluoro-1-oxo-1,2-dihydroisoquinolin-4-yl)ethyl)-6-fluoro-N-methyl-1H-indole-2-carboxamide—Enantiomer I (Compound 11), LCMS: m/z found 400.3 [M+H]⁺, RT=4.12 min (Method A); ¹H NMR (400 MHz, DMSO-d₆): δ 11.75 (bs, 2H), 8.14-8.09 (m, 1H), 7.63-7.59 (m, 2H), 7.34 (s, 1H), 7.19-7.15 (m, 1H), 6.94-6.88 (m, 2H), 6.13-6.09 (m, 1H), 2.93 (s, 3H), 1.53 (d, 3H); Chiral analytical SFC: RT=2.21 min, Column: Chiralpak IC-3, (4.6×150 mm, 3 μm), 60% CO₂/MeOH, Flow=3.0 g/min.

N-(1-(6,7-Difluoro-1-oxo-1,2-dihydroisoquinolin-4-yl)ethyl)-6-fluoro-N-methyl-1H-indole-2-carboxamide—Enantiomer II (Compound 12), LCMS: m/z found 400.3, [M+H]⁺, RT=4.12 min (Method A); ¹H NMR (400 MHz, DMSO-d₆): δ 11.75 (bs, 2H), 8.14-8.09 (m, 1H), 7.63-7.59 (m, 2H), 7.34 (s, 1H), 7.19-7.15 (m, 1H), 6.94-6.88 (m, 2H), 6.13-6.09 (m, 1H), 2.93 (s, 3H), 1.53 (d, 3H); Chiral analytical SFC: RT=3.18 min, Column: Chiralpak IC-3, (4.6×150 mm, 3 μm), 60% CO₂/MeOH, Flow=3.0 g/min.

### N-(1-(6,7-Difluoro-1-oxo-1,2-dihydroisoquinolin-4-yl)ethyl)-7-fluoro-N-methyl-1H-indole-2-carboxamide (Compounds 13 & 14)

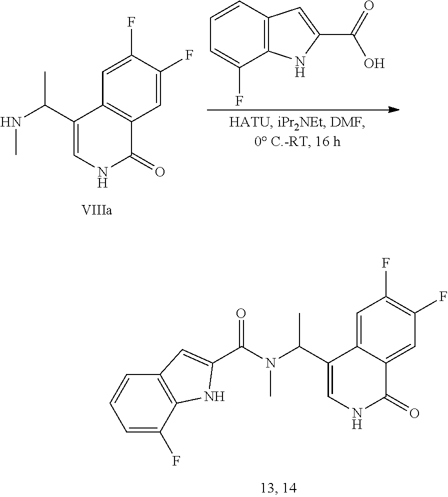

Racemic N-(1-(6,7-difluoro-1-oxo-1,2-dihydroisoquinolin-4-yl)ethyl)-7-fluoro-N-methyl-1H-indole-2-carboxamide was synthesized in a similar manner as described above from 1H-6,7-difluoro-4-(1-(methylamino)ethyl)isoquinolin-1(2H)-one (VIIIa) and 7-fluoro-1H-indole-2-carboxylic acid. The enantiomers were subsequently separated by chiral SFC, Column: Chiralpak IC (250×30 mm, 5 μm) 55% CO₂/MeOH, Flow rate 100 g/min.

N-(1-(6,7-Difluoro-1-oxo-1,2-dihydroisoquinolin-4-yl)ethyl)-7-fluoro-N-methyl-1H-indole-2-carboxamide—Enantiomer I (Compound 13), LCMS: m/z found 400.3 [M+H]⁺, RT=4.10 min (Method A); ¹H NMR (400 MHz, DMSO-d₆): δ 12.11 (bs, 1H), 11.72 (bs, 1H), 8.16-8.11 (m, 1H), 7.62-7.56 (m, 1H), 7.42-7.38 (m, 1H), 7.32 (s, 1H), 7.05-6.99 (m, 2H), 6.91-6.90 (m, 1H), 6.12-6.06 (m, 1H), 2.87 (s, 3H), 1.55 (d, 3H); Chiral analytical SFC: RT=2.48 min, Column: Chiralpak IC-3, (4.6×150 mm, 3 μm), 60% CO₂/MeOH, Flow=3.0 g/min.

N-(1-(6,7-Difluoro-1-oxo-1,2-dihydroisoquinolin-4-yl)ethyl)-7-fluoro-N-methyl-1H-indole-2-carboxamide—Enantiomer II (Compound 14), LCMS: m/z found 400.3 [M+H]⁺, RT=4.10 min (Method A); ¹H NMR (400 MHz, DMSO-d₆): δ 12.11 (bs, 1H), 11.72 (bs, 1H), 8.16-8.11 (m, 1H), 7.62-7.56 (m, 1H), 7.42-7.38 (m, 1H), 7.32 (s, 1H), 7.05-6.99 (m, 2H), 6.91-6.90 (m, 1H), 6.12-6.06 (m, 1H), 2.87 (s, 3H), 1.55 (d, 3H); Chiral analytical SFC: RT=4.37 min, Column: Chiralpak IC-3, (4.6×150 mm, 3 μm), 60% CO₂/MeOH, Flow=3.0 g/min.

### N-(1-(6,7-Difluoro-1-oxo-1,2-dihydroisoquinolin-4-yl)ethyl)-5,6-difluoro-N-methyl-1H-indole-2-carboxamide (Compounds 15 & 16)

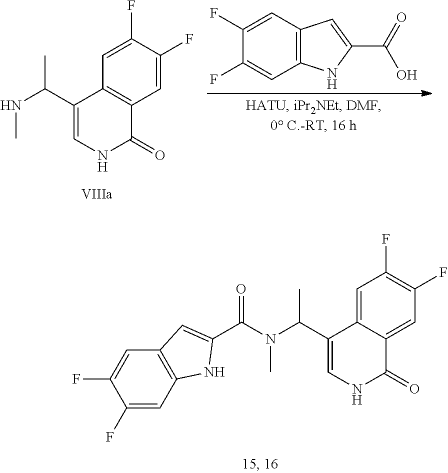

Racemic N-(1-(6,7-difluoro-1-oxo-1,2-dihydroisoquinolin-4-yl)ethyl)-5,6-difluoro-N-methyl-1H-indole-2-carboxamide was synthesized in a similar manner as described above from 1H-6,7-difluoro-4-(1-(methylamino)ethyl)isoquinolin-1(2H)-one (VIIIa) and 5,6-difluoro-1H-indole-2-carboxylic acid. The enantiomers were subsequently separated by chiral SFC, Column: Chiralpak IC (250×30 mm, 5 μm) 75% CO₂/MeOH, Flow rate 100 g/min.

N-(1-(6,7-Difluoro-1-oxo-1,2-dihydroisoquinolin-4-yl)ethyl)-5,6-difluoro-N-methyl-1H-indole-2-carboxamide—Enantiomer I (Compound 15), LCMS: m/z found 418.2 [M+H]⁺, RT=4.27 min (Method A); ¹H NMR (400 MHz, DMSO-d₆): δ 11.89 (bs, 1H), 11.70 (bs, 1H), 8.15-8.10 (m, 1H), 7.61-7.56 (m, 2H), 7.39-7.33 (m, 2H), 6.90 (s, 1H), 6.12-6.08 (m, 1H), 2.92 (s, 3H), 1.53 (d, 3H); Chiral analytical SFC: RT=2.28 min, Column: Chiralpak IC-3, (4.6×150 mm, 3 μm), 65% CO₂/MeOH, Flow=3.0 g/min.

N-(1-(6,7-Difluoro-1-oxo-1,2-dihydroisoquinolin-4-yl)ethyl)-5,6-difluoro-N-methyl-1H-indole-2-carboxamide—Enantiomer II (Compound 16), LCMS: m/z found 418.2 [M+H]⁺, RT=4.27 min (Method A); ¹H NMR (400 MHz, DMSO-d₆): δ 11.89 (bs, 1H), 11.70 (bs, 1H), 8.15-8.10 (m, 1H), 7.61-7.56 (m, 2H), 7.39-7.33 (m, 2H), 6.90 (s, 1H), 6.12-6.08 (m, 1H), 2.92 (s, 3H), 1.53 (d, 3H); Chiral analytical SFC: RT=2.98 min, Column: Chiralpak IC-3, (4.6×150 mm, 3 μm), 65% CO₂/MeOH, Flow=3.0 g/min.

### N-(1-(6,7-Difluoro-1-oxo-1,2-dihydroisoquinolin-4-yl)ethyl)-4,6-difluoro-N-methyl-1H-indole-2-carboxamide (Compounds 17 & 18)

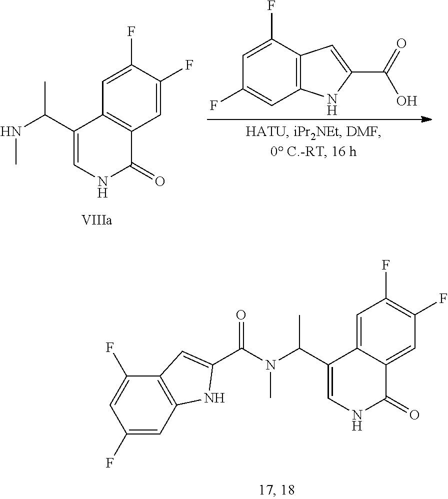 [SMILES: CNC(C)c1c[nH]c(=O)c2cc(F)c(F)cc12 ; O=C(O)c1cc2c(F)cc(F)cc2[nH]1 ; CC(c1c[nH]c(=O)c2cc(F)c(F)cc12)N(C)C(=O)c1cc2c(F)cc(F)cc2[nH]1]

Racemic N-(1-(6,7-difluoro-1-oxo-1,2-dihydroisoquinolin-4-yl)ethyl)-4,6-difluoro-N-methyl-1H-indole-2-carboxamide was synthesized in a similar manner as described above from 1H-6,7-difluoro-4-(1-(methylamino)ethyl)isoquinolin-1(2H)-one (VIIIa) and 4,6-difluoro-1H-indole-2-carboxylic acid. The enantiomers were subsequently separated by chiral SFC, Column: Chiralpak IC (250×30 mm, 5 μm) 75% CO₂/MeOH, Flow rate 90 g/min.

N-(1-(6,7-Difluoro-1-oxo-1,2-dihydroisoquinolin-4-yl)ethyl)-4,6-difluoro-N-methyl-1H-indole-2-carboxamide—Enantiomer I (Compound 17), LCMS: m/z found 418.2 [M+H]⁺, RT=4.38 min (Method A); ¹H NMR (400 MHz, DMSO-d₆): δ 11.92 (bs, 2H), 8.15-8.10 (m, 1H), 7.63-7.61 (m, 1H), 7.34 (s, 1H), 7.07-7.04 (m, 1H), 6.95 (s, 1H), 6.91-6.85 (m, 1H), 6.12-6.09 (m, 1H), 2.95 (s, 3H), 1.53 (d, 3H); Chiral analytical SFC: RT=2.05 min, Column: Chiralpak IC-3, (4.6×150 mm, 3 μm), 75% CO₂/MeOH, Flow=3.0 g/min.

N-(1-(6,7-Difluoro-1-oxo-1,2-dihydroisoquinolin-4-yl)ethyl)-4,6-difluoro-N-methyl-1H-indole-2-carboxamide—Enantiomer II (Compound 18), LCMS: m/z found 418.2 [M+H]⁺, RT=4.38 min (Method A); ¹H NMR (400 MHz, DMSO-d₆): δ 11.92 (bs, 2H), 8.15-8.10 (m, 1H), 7.63-7.61 (m, 1H), 7.34 (s, 1H), 7.07-7.04 (m, 1H), 6.95 (s, 1H), 6.91-6.85 (m, 1H), 6.12-6.09 (m, 1H), 2.95 (s, 3H), 1.53 (d, 3H); Chiral analytical SFC: RT=2.58 min, Column: Chiralpak IC-3, (4.6×150 mm, 3 μm), 75% CO₂/MeOH, Flow=3.0 g/min.

### N-(1-(6,7-Difluoro-1-oxo-1,2-dihydroisoquinolin-4-yl)ethyl)-N-(3-hydroxypropyl)-1H-indole-2-carboxamide (Compounds 27 & 28)

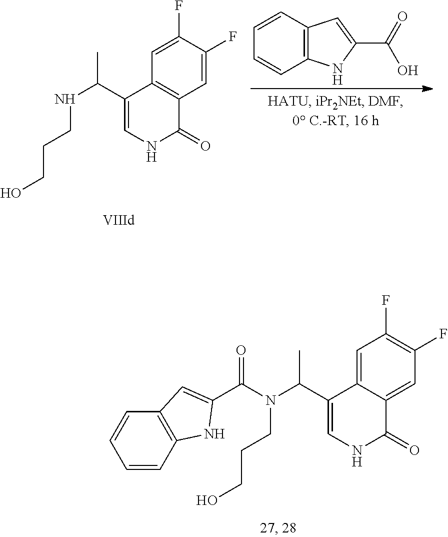

Racemic N-(1-(6,7-difluoro-1-oxo-1,2-dihydroisoquinolin-4-yl)ethyl)-N-(3-hydroxypropyl)-1H-indole-2-carboxamide was synthesized in a similar manner as described above from 6,7-difluoro-4-(1-((3-hydroxypropyl)amino)ethyl)isoquinolin-1(2H)-one (VIIId) and 1H-indole-2-carboxylic acid. The enantiomers were subsequently separated by chiral SFC, Column: Chiralpak IC (250×30 mm, 5 m) 60% CO₂/MeOH, Flow rate 90 g/min.

N-(1-(6,7-Difluoro-1-oxo-1,2-dihydroisoquinolin-4-yl)ethyl)-N-(3-hydroxypropyl)-1H-indole-2-carboxamide—Enantiomer I (Compound 27), LCMS: m/z found 426.3 [M+H]⁺, RT=4.68 min (Method A); ¹H NMR (400 MHz, DMSO-d₆): δ 11.71 (bs, 2H), 8.14-8.09 (m, 1H), 7.64-7.57 (m, 2H), 7.47 (d, 1H), 7.39 (s, 1H), 7.22-7.18 (m, 1H), 7.05-7.01 (m, 1H), 6.99 (s, 1H), 6.18-6.14 (m, 1H), 4.47 (bs, 1H), 3.53-3.31 (m, 2H), 3.24-3.18 (m, 2H), 1.60-1.45 (m, 4H), 1.06-1.01 (m, 1H); Chiral analytical SFC: RT=2.23 min, Column: Chiralpak IC, (4.6×150 mm, 3 μm), 60% CO₂/MeOH, Flow=4.0 g/min.

N-(1-(6,7-Difluoro-1-oxo-1,2-dihydroisoquinolin-4-yl)ethyl)-N-(3-hydroxypropyl)-1H-indole-2-carboxamide—Enantiomer II (Compound 28), LCMS: m/z found 426.3 [M+H]⁺, RT=4.68 min (Method A); ¹H NMR (400 MHz, DMSO-d₆): δ 11.71 (bs, 2H), 8.14-8.09 (m, 1H), 7.64-7.57 (m, 2H), 7.47 (d, 1H), 7.39 (s, 1H), 7.22-7.18 (m, 1H), 7.05-7.01 (m, 1H), 6.99 (s, 1H), 6.18-6.14 (m, 1H), 4.47 (bs, 1H), 3.53-3.31 (m, 2H), 3.24-3.18 (m, 2H), 1.60-1.45 (m, 4H), 1.06-1.01 (m, 1H); Chiral analytical SFC: RT=4.46 min, Column: Chiralpak IC, (4.6×150 mm, 3 μm), 60% CO₂/MeOH, Flow=4.0 g/min.

### N-(1-(6,7-Difluoro-1-oxo-1,2-dihydroisoquinolin-4-yl)ethyl)-4,5-difluoro-N-methyl-1H-indole-2-carboxamide (Compound 29)

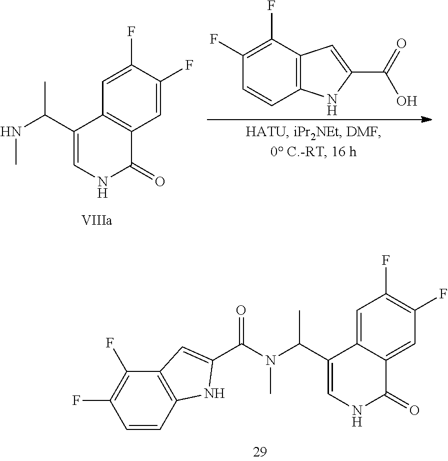

Racemic N-(1-(6,7-difluoro-1-oxo-1,2-dihydroisoquinolin-4-yl)ethyl)-4,5-difluoro-N-methyl-1H-indole-2-carboxamide (Compound 29) was synthesized in a similar manner as described above from 1H-6,7-difluoro-4-(1-(methylamino)ethyl)isoquinolin-1(2H)-one (VIIIa) and 4,5-difluoro-1H-indole-2-carboxylic acid. LCMS: m/z found 418.2 [M+H]⁺, RT=4.38 min (Method A); ¹H NMR (400 MHz, DMSO-d₆): δ 12.11 (bs, 1H), 11.71 (bs, 1H), 8.16-8.11 (m, 1H), 7.63-7.59 (m, 1H), 7.34-7.21 (m, 3H), 6.99 (s, 1H), 6.14-6.09 (m, 1H), 2.94 (s, 3H), 1.54 (d, 3H).

### N-(1-(6,7-Difluoro-1-oxo-1,2-dihydroisoquinolin-4-yl)ethyl)-N,1-dimethyl-1H-indole-2-carboxamide (Compounds 43 & 44)

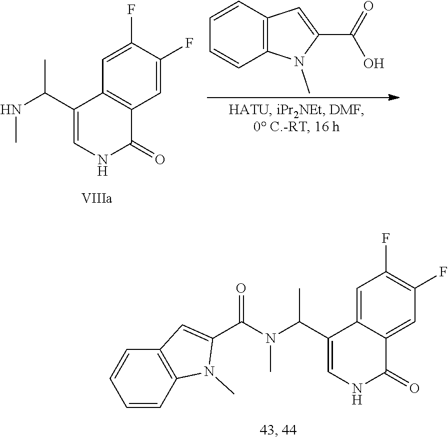

Racemic N-(1-(6,7-difluoro-1-oxo-1,2-dihydroisoquinolin-4-yl)ethyl)-N,1-dimethyl-1H-indole-2-carboxamide was synthesized in a similar manner as described above from 1H-6,7-difluoro-4-(1-(methylamino)ethyl)isoquinolin-1(2H)-one (VIIIa) and 1-methyl-1H-indole-2-carboxylic acid. The enantiomers were subsequently separated by chiral SFC, Column: Chiralpak IC (250×30 mm, 5 μm) 60% CO₂/MeOH, Flow rate 100 g/min.

N-(1-(6,7-Difluoro-1-oxo-1,2-dihydroisoquinolin-4-yl)ethyl)-N,1-dimethyl-1H-indole-2-carboxamide—Enantiomer I (Compound 43), LCMS: m/z found 396.3 [M+H]⁺, RT=4.26 min (Method A); ¹H NMR (400 MHz, DMSO-d₆): δ 11.41 (bs, 1H), 8.13-8.07 (m, 1H), 7.58-7.53 (m, 2H), 7.47 (d, 1H), 7.30 (s, 1H), 7.25-7.21 (m, 1H), 7.09-7.05 (m, 1H), 6.60 (s, 1H), 6.08-6.03 (m, 1H), 3.76 (s, 3H), 2.70 (s, 3H), 1.60 (d, 3H); Chiral analytical SFC: RT=3.86 min, Column: Chiralpak IC-3, (4.6×150 mm, 3 μm), 60% CO₂/MeOH, Flow=3.0 g/min.

N-(1-(6,7-Difluoro-1-oxo-1,2-dihydroisoquinolin-4-yl)ethyl)-N,1-dimethyl-1H-indole-2-carboxamide—Enantiomer II (Compound 44), LCMS: m/z found 396.3 [M+H]⁺, RT=4.26 min (Method A); ¹H NMR (400 MHz, DMSO-d₆): δ 11.41 (bs, 1H), 8.13-8.07 (m, 1H), 7.58-7.53 (m, 2H), 7.47 (d, 1H), 7.30 (s, 1H), 7.25-7.21 (m, 1H), 7.09-7.05 (m, 1H), 6.60 (s, 1H), 6.08-6.03 (m, 1H), 3.76 (s, 3H), 2.70 (s, 3H), 1.60 (d, 3H); Chiral analytical SFC: RT=6.66 min, Column: Chiralpak IC-3, (4.6×150 mm, 3 μm), 60% CO₂/MeOH, Flow=3.0 g/min.

### 3-Chloro-N-(1-(6,7-difluoro-1-oxo-1,2-dihydroisoquinolin-4-yl)ethyl)-N-methyl-1H-indole-2-carboxamide (Compound 60)

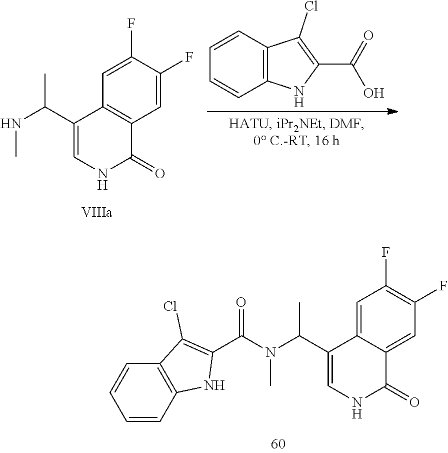

Racemic 3-chloro-N-(1-(6,7-difluoro-1-oxo-1,2-dihydroisoquinolin-4-yl)ethyl)-N-methyl-1H-indole-2-carboxamide (Compound 60) was synthesized in a similar manner as described above from 1H-6,7-difluoro-4-(1-(methylamino)ethyl)isoquinolin-1(2H)-one (VIIIa) and 3-chloro-1H-indole-2-carboxylic acid. LCMS: m/z found 416.2/418.2 [M+H]⁺, RT=4.28 min (Method A); ¹H NMR (400 MHz, DMSO-d₆): δ 11.97 (bs, 1H), 11.70 (bs, 1H), 8.18-8.13 (m, 1H), 7.63-7.60 (m, 1H), 7.49 (d, 1H), 7.43 (d, 1H), 7.32 (s, 1H), 7.28-7.24 (m, 1H), 7.17-7.13 (m, 1H), 6.03-5.99 (m, 1H), 2.64 (s, 3H), 1.58 (d, 3H).

### N-(1-(6,7-Difluoro-1-oxo-1,2-dihydroisoquinolin-4-yl)ethyl)-N-methyl-1H-pyrrolo[2,3-b]pyridine-2-carboxamide (Compounds 51 & 52)

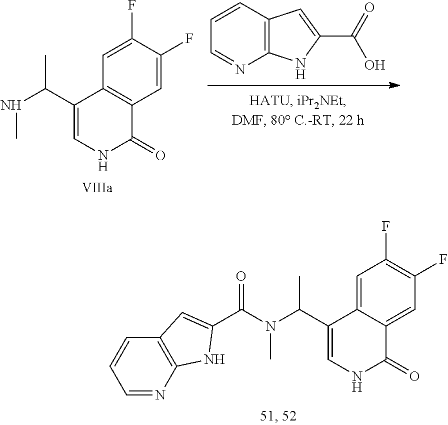

Racemic N-(1-(6,7-difluoro-1-oxo-1,2-dihydroisoquinolin-4-yl)ethyl)-N-methyl-1H-pyrrolo[2,3-b]pyridine-2-carboxamide was synthesized in a similar manner as described above from 1H-6,7-difluoro-4-(1-(methylamino)ethyl)isoquinolin-1(2H)-one (VIIIa) and 1H-pyrrolo[2,3-b]pyridine-2-carboxylic acid. The enantiomers were subsequently separated by chiral SFC, Column: Chiralpak IA (250×30 mm, 5 μm) 50% CO₂/MeOH, Flow rate 75 g/min.

N-(1-(6,7-Difluoro-1-oxo-1,2-dihydroisoquinolin-4-yl)ethyl)-N-methyl-1H-pyrrolo[2,3-b]pyridine-2-carboxamide—Enantiomer I (Compound 51), LCMS: m/z found 383.2 [M+H]⁺, RT=2.41 min (Method A); ¹H NMR (400 MHz, DMSO-d₆): δ 12.23 (bs, 1H), 11.71 (bs, 1H), 8.34 (bs, 1H), 8.16-8.11 (m, 1H), 8.03 (d, 1H), 7.57 (bs, 1H), 7.32 (d, 1H), 7.14-7.11 (m, 1H), 6.82 (s, 1H), 6.08-6.02 (m, 1H), 2.86 (s, 3H), 1.54 (d, 3H); Chiral analytical SFC: RT=3.55 min, Column: Chiralpak IA-3, (4.6×150 mm, 3 μm), 60% CO₂/MeOH, Flow=3.0 g/min.

N-(1-(6,7-Difluoro-1-oxo-1,2-dihydroisoquinolin-4-yl)ethyl)-N-methyl-1H-pyrrolo[2,3-b]pyridine-2-carboxamide—Enantiomer II (Compound 52), LCMS: m/z found 383.2 [M+H]⁺, RT=2.41 min (Method A); ¹H NMR (400 MHz, DMSO-d₆): δ 12.23 (bs, 1H), 11.71 (bs, 1H), 8.34 (bs, 1H), 8.16-8.11 (m, 1H), 8.03 (d, 1H), 7.57 (bs, 1H), 7.32 (d, 1H), 7.14-7.11 (m, 1H), 6.82 (s, 1H), 6.08-6.02 (m, 1H), 2.86 (s, 3H), 1.54 (d, 3H); Chiral analytical SFC: RT=9.32 min, Column: Chiralpak IA-3, (4.6×150 mm, 3 μm), 60% CO₂/MeOH, Flow=3.0 g/min.

### 3-Chloro-N-(1-(6,7-difluoro-1-oxo-1,2-dihydroisoquinolin-4-yl)ethyl)-N-(3-hydroxypropyl)-1H-indole-2-carboxamide (Compounds 94 & 95)

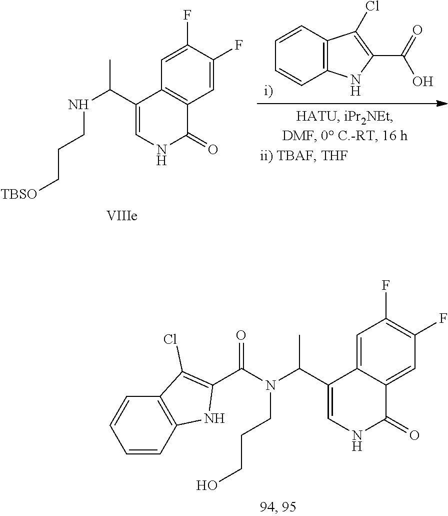

Racemic N-(3-((tert-butyldimethylsilyl)oxy)propyl)-3-chloro-N-(1-(6,7-difluoro-1-oxo-1,2-dihydroisoquinolin-4-yl)ethyl)-1H-indole-2-carboxamide was synthesized in a similar manner as described above from 4-(1-((3-((tert-butyldimethylsilyl)oxy)propyl)amino)ethyl)-6,7-difluoroisoquinolin-1(2H)-one (VIIIe) and 3-chloro-1H-indole-2-carboxylic acid. To a stirred solution of 0.14 g (0.24 mmol. 1.0 eq.) of the racemic N-(3-((tert-butyldimethylsilyl)oxy)propyl)-3-chloro-N-(1-(6,7-difluoro-1-oxo-1,2-dihydroisoquinolin-4-yl)ethyl)-1H-indole-2-carboxamide in 3 mL of THF at 0° C. was added 0.48 mL (0.48 mmol, 2.0 eq.) of a 1.0 M solution of tetrabutyl ammonium fluoride in THE and the mixture was stirred at room temperature for 2 h. The reaction mixture was then diluted with 1 mL of methanol and the solvent was removed in vacuo. The residue was triturated with water and the enantiomers were subsequently separated by chiral SFC, Column: Chiralpak IC (250×30 mm, 5 μm) 60% CO₂/MeOH, Flow rate 100 g/min.

3-Chloro-N-(1-(6,7-difluoro-1-oxo-1,2-dihydroisoquinolin-4-yl)ethyl)-N-(3-hydroxypropyl)-1H-indole-2-carboxamide—Enantiomer I (Compound 94), LCMS: m/z found 460.2/462.2 [M+H]⁺, RT=3.90 min (Method A); ¹H NMR (400 MHz, DMSO-d₆): δ 11.72 (bs 2H), 8.17-8.12 (m, 1H), 7.73-7.68 (m, 1H), 7.48 (d, 1H), 7.42 (d, 1H), 7.37 (s, 1H), 7.22 (t, 1H) 7.12 (t, 1H), 6.05 (s, 1H), 4.45 (bs, 1H), 3.32-3.11 (m, 2H), 2.91-2.87 (m, 2H), 1.61 (d, 3H), 1.33-1.28 (m, 1H), 0.94-0.92 (m, 1H); Chiral analytical SFC: RT=1.57 min, Column: Chiralpak IC-3, (4.6×150 mm, 3 m), 60% CO₂/MeOH, Flow=3.0 g/min.

3-Chloro-N-(1-(6,7-difluoro-1-oxo-1,2-dihydroisoquinolin-4-yl)ethyl)-N-(3-hydroxypropyl)-1H-indole-2-carboxamide—Enantiomer II (Compound 95), LCMS: m/z found 460.2/462.2 [M+H]⁺, RT=3.90 min (Method A); ¹H NMR (400 MHz, DMSO-d₆): δ 11.72 (bs 2H), 8.17-8.12 (m, 1H), 7.73-7.68 (m, 1H), 7.48 (d, 1H), 7.42 (d, 1H), 7.37 (s, 1H), 7.22 (t, 1H) 7.12 (t, 1H), 6.05 (s, 1H), 4.45 (bs, 1H), 3.32-3.11 (m, 2H), 2.91-2.87 (m, 2H), 1.61 (d, 3H), 1.33-1.28 (m, 1H), 0.94-0.92 (m, 1H); Chiral analytical SFC: RT=2.47 min, Column: Chiralpak IC-3, (4.6×150 mm, 3 μm), 60% CO₂/MeOH, Flow=3.0 g/min.

### (3S)-N-(1-(6,7-Difluoro-1-oxo-1,2-dihydroisoquinolin-4-yl)ethyl)-N-methyl-1,2,3,4-tetrahydro isoquinoline-3-carboxamide (Compounds 19 & 20)

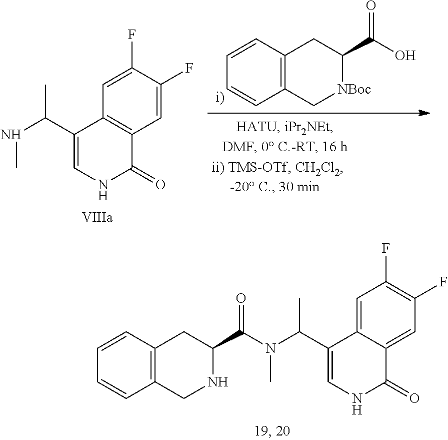

tert-Butyl (3S)-3-((1-(6,7-difluoro-1-oxo-1,2-dihydroisoquinolin-4-yl)ethyl)(methyl) carbamoyl)-3,4-dihydroisoquinoline-2(1H)-carboxylate as a mixture of two diastereoisomers was synthesized in a similar manner as described above from 1H-6,7-difluoro-4-(1-(methylamino)ethyl)isoquinolin-1(2H)-one (VIIIa) and (S)-2-(tert-butoxycarbonyl)-1,2,3,4-tetrahydroisoquinoline-3-carboxylic acid. To a stirred solution of 0.14 g (0.28 mmol, 1.0 eq.) of diastereomeric tert-butyl (3S)-3-((1-(6,7-difluoro-1-oxo-1,2-dihydroisoquinolin-4-yl)ethyl)(methyl)carbamoyl)-3,4-dihydroisoquinoline-2(1H)-carboxylate in 3 mL of methylene chloride at −20° C. under a nitrogen atmosphere was added 0.1 mL (0.56 mmol, 2.0 eq.) of trimethylsilyl trifluoromethansulfonate (TMSOTf) and the mixture was stirred at for 30 min. The reaction mixture was then basified with 30 mL of saturated sodium bicarbonate solution and extracted with ethyl acetate (3×50 mL). The combined organic extracts were washed with brine (50 mL), dried (Na₂SO₄) and the solvent was removed in vacuo. The residue was triturated with diethyl ether (6 mL) at room temperature and the solid was collected by filtration and dried under vacuum to provide 100 mg (0.25 mmol, 90% yield) of (3S)-N-(1-(6,7-difluoro-1-oxo-1,2-dihydroisoquinolin-4-yl)ethyl)-N-methyl-1,2,3,4-tetrahydroisoquinoline-3-carboxamide. The diastereoisomers were subsequently separated by chiral SFC, Column: Chiralpak IG (250×30 mm, 5 μm) 60% CO₂/(15 mM ammonia in Methanol), Flow rate 90 g/min.

(3S)-N-(1-(6,7-Difluoro-1-oxo-1,2-dihydroisoquinolin-4-yl)ethyl)-N-methyl-1,2,3,4-tetrahydro isoquinoline-3-carboxamide—Diastereoisomer I (Compound 19), LCMS: m/z found 398.3 [M+H]⁺, RT=1.98 min (Method A); ¹H NMR (400 MHz, DMSO-d₆): δ 11.64 (bs, 1H), 8.13-8.08 (m, 1H), 7.61-7.56 (m, 1H), 7.24 (s, 1H), 7.11-7.08 (m, 3H), 7.01-6.99 (m, 1H), 5.99-5.94 (m, 1H), 3.90-3.79 (m, 3H), 2.88-2.74 (m, 2H), 2.72-2.67 (m, 3H), 2.31-2.27 (bs, 1H), 1.41 (d, 3H); Chiral analytical SFC: RT=3.28 min, Column: Chiralpak IG-3, (4.6×150 mm, 3 m), 60% CO₂/(0.5% DEA in Methanol), Flow=3.0 g/min.

(3S)-N-(1-(6,7-Difluoro-1-oxo-1,2-dihydroisoquinolin-4-yl)ethyl)-N-methyl-1,2,3,4-tetrahydro isoquinoline-3-carboxamide—Diastereoisomer II (Compound 20), LCMS: m/z found 398.3 [M+H]⁺, RT=2.38 min (Method A); ¹H NMR (400 MHz, DMSO-d₆): δ 11.64 (bs, 1H), 8.15-8.10 (m, 1H), 7.49-7.44 (m, 1H), 7.24 (s, 1H), 7.13-7.01 (m, 4H), 5.97-5.93 (m, 1H), 3.93-3.84 (m, 3H), 2.77-2.71 (m, 4H), 2.61-2.54 (m, 1H), 2.42 (bs, 1H), 1.41 (d, 3H); Chiral analytical SFC: RT=4.49 min, Column: Chiralpak IG-3, (4.6×150 mm, 3 m), 60% CO₂/(0.5% DEA in Methanol), Flow=3.0 g/min.

### (3R)-N-(1-(6,7-Difluoro-1-oxo-1,2-dihydroisoquinolin-4-yl)ethyl)-N-methyl-1,2,3,4-tetrahydro-isoquinoline-3-carboxamide (Compounds 32 & 33)

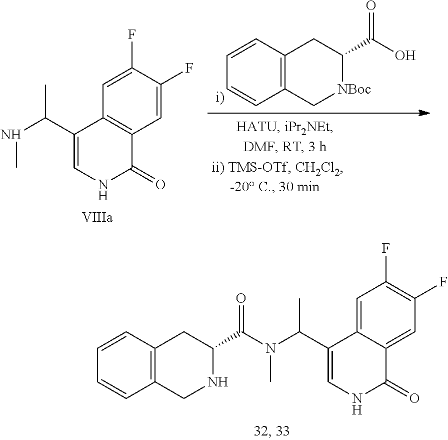

(3R)-N-(1-(6,7-Difluoro-1-oxo-1,2-dihydroisoquinolin-4-yl)ethyl)-N-methyl-1,2,3,4-tetrahydro isoquinoline-3-carboxamide as a mixture of two diastereoisomers was synthesized in a similar manner as described above from 1H-6,7-difluoro-4-(1-(methylamino)ethyl)isoquinolin-1(2H)-one (VIIIa) and tert-butyl (3R)-3-((1-(6,7-difluoro-1-oxo-1,2-dihydroisoquinolin-4-yl)ethyl)(methyl)carbamoyl)-3,4-dihydroisoquinoline-2(1H)-carboxylate. The diastereoisomers were separated by preparative HPLC (Column: Luna (25×150 mm, 10 μm), Mobile phase A: 100% water, Mobile phase B: methanol; Method T/% B=0/60, 11/85, 13/90, 13.1/100, 15/100, 15.1/60; Flow rate: 19 mL/min). Pure fractions were concentrated under reduced pressure and lyophilized, then each diastereoisomer was further purified by chiral SFC, Column: Chiralcel OD-H (250×21 mm, 5 μm) 65% CO₂/(30 mM methanolic ammonia in ethanol), Flow rate 90 g/min, and 60 g/min, respectively.

(3R)-N-(1-(6,7-Difluoro-1-oxo-1,2-dihydroisoquinolin-4-yl)ethyl)-N-methyl-1,2,3,4-tetrahydro isoquinoline-3-carboxamide—Diastereoisomer I (Compound 32), LCMS: m/z found 398.3 [M+H]⁺, RT=1.99 min (Method A); ¹H NMR (400 MHz, DMSO-d₆): 11.61 (bs, 1H), 8.14-8.08 (m, 1H), 7.61-7.56 (m, 1H), 7.24 (s, 1H), 7.12-7.07 (m, 3H), 7.01-6.98 (m, 1H), 5.98-5.94 (m, 1H), 3.91-3.79 (m, 3H), 2.88-2.68 (m, 5H), 2.56-2.51 (m, 1H), 1.41 (d, 3H); Chiral analytical SFC: RT=2.02 min, Column: Chiralcel OD-3, (4.6×150 mm, 3 m), 70% CO₂/(0.5% DEA in Ethanol), Flow=3.0 g/min.

(3R)-N-(1-(6,7-Difluoro-1-oxo-1,2-dihydroisoquinolin-4-yl)ethyl)-N-methyl-1,2,3,4-tetrahydro isoquinoline-3-carboxamide—Diastereoisomer II (Compound 33), LCMS: m/z found 398.3 [M+H]⁺, RT=2.37 min (Method A); ¹H NMR (400 MHz, DMSO-d₆): δ 11.61 (bs, 1H), 8.15-8.10 (m, 1H), 7.50-7.44 (m, 1H), 7.25-7.23 (m, 1H), 7.14-7.01 (m, 4H), 5.98-5.93 (m, 1H), 3.98-3.83 (m, 3H), 2.77-2.71 (m, 1H), 2.68 (s, 3H), 2.62-2.57 (m, 1H), 2.48 (br s, 1H), 1.41 (d, 3H); Chiral analytical SFC: RT=2.55 min, Column: Chiralcel OD-3, (4.6×150 mm, 3 m), 70% CO₂/(0.5% DEA in Ethanol), Flow=3.0 g/min.

### (2R)-N-(1-(6,7-Difluoro-1-oxo-1,2-dihydroisoquinolin-4-yl)ethyl)-N-methylindoline-2-carboxamide (Compounds 21 & 22)

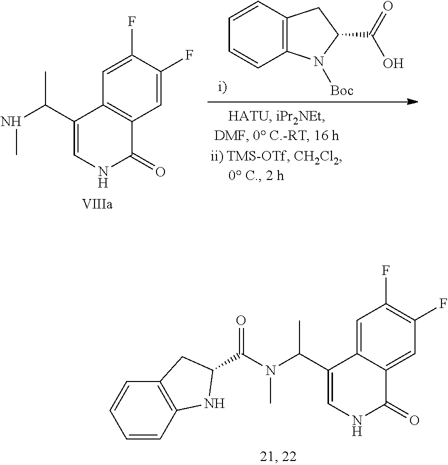

Diastereomeric (2R)-N-(1-(6,7-difluoro-1-oxo-1,2-dihydroisoquinolin-4-yl)ethyl)-N-methylindoline-2-carboxamide (mixture of two diastereoisomers) was synthesized in a similar manner as described above from 1H-6,7-difluoro-4-(1-(methylamino)ethyl)isoquinolin-1(2H)-one (VIIIa) and (R)-1-(tert-butoxycarbonyl)indoline-2-carboxylic acid. The diastereoisomers were subsequently separated by MPLC (Silica gel column-24 g, eluting with 0-3.5% gradient of methanol in methylene chloride).

(2R)-N-(1-(6,7-Difluoro-1-oxo-1,2-dihydroisoquinolin-4-yl)ethyl)-N-methylindoline-2-carboxamide—Diastereoisomer I (Compound 21), LCMS: m/z found 384.3 [M+H]⁺, RT=2.77 min (Method A); ¹H NMR (400 MHz, DMSO-d₆): δ 11.64 (bs, 1H), 8.14-8.09 (m, 1H), 7.28-7.22 (m, 2H), 6.96-6.92 (m, 2H), 6.60-6.53 (m, 2H), 5.89-5.83 (m, 1H), 5.75 (s, 1H), 4.66-4.61 (m, 1H), 3.27-3.20 (m, 1H), 2.80-2.74 (m, 1H), 2.61 (s, 3H), 1.41 (d, 3H); Chiral analytical SFC: RT=2.74 min, Column: Chiralpak AD-3, (4.6×150 mm, 3 m), 60% CO₂/(0.5% DEA in Ethanol), Flow=3.0 g/min.

(2R)-N-(1-(6,7-Difluoro-1-oxo-1,2-dihydroisoquinolin-4-yl)ethyl)-N-methylindoline-2-carboxamide—Diastereoisomer II (Compound 22), LCMS: m/z found 384.3 [M+H]⁺, RT=2.71 min (Method A); ¹H NMR (400 MHz, DMSO-d₆): δ 11.64 (bs, 1H), 8.14-8.09 (m, 1H), 7.56-7.51 (m, 1H), 7.23 (s, 1H), 7.01-6.99 (m, 1H), 6.94-6.91 (m, 1H), 6.57-6.53 (m, 2H), 5.93-5.90 (m, 1H), 5.75-5.74 (m, 1H), 4.64-4.59 (m, 1H), 3.35-3.28 (m, 1H), 3.17-3.08 (m, 1H), 2.65 (s, 3H), 1.38 (d, 3H); Chiral analytical SFC: RT=4.30 min, Column: Chiralpak AD-3, (4.6×150 mm, 3 m), 60% CO₂/(0.5% DEA in Ethanol), Flow=3.0 g/min.

### (2S)-N-(1-(6,7-Difluoro-1-oxo-1,2-dihydroisoquinolin-4-yl)ethyl)-N-methylindoline-2-carboxamide (Compounds 30 & 31)

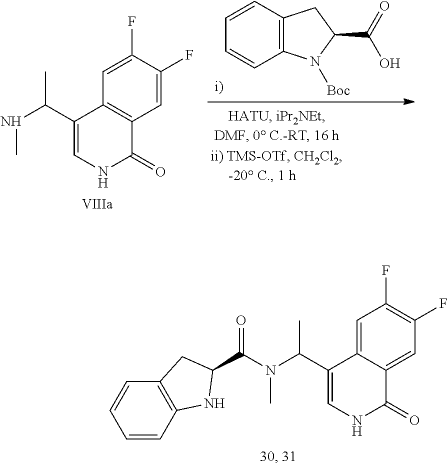

Diastereomeric (2S)-N-(1-(6,7-difluoro-1-oxo-1,2-dihydroisoquinolin-4-yl)ethyl)-N-methylindoline-2-carboxamide was synthesized in a similar manner as described above from 1H-6,7-difluoro-4-(1-(methylamino)ethyl)isoquinolin-1(2H)-one (VIIIa) and (S)-1-(tert-butoxycarbonyl)indoline-2-carboxylic acid. The diastereoisomers were subsequently separated by MPLC (Silica gel column 24 g, eluting with 0-5% gradient of methanol in dichloromethane). Diastereoisomer II was further purified by preparative HPLC (Column: Luna (25×150 mm, 10 μm), Mobile phase A: 10 mM ammonium bicarbonate in water, Mobile phase B: Acetonitrile; Method T/% B=0/40, 11/60, 11.1/100, 13/100, 13.1/50; Flow rate: 19 mL/min).

(2S)-N-(1-(6,7-Difluoro-1-oxo-1,2-dihydroisoquinolin-4-yl)ethyl)-N-methylindoline-2-carboxamide—Diastereoisomer I (Compound 30), LCMS: m/z found 384.3 [M+H]⁺, RT=2.71 min (Method A); ¹H NMR (400 MHz, DMSO-d₆): δ 11.65 (bs, 1H), 8.14-8.09 (m, 1H), 7.56-7.51 (m, 1H), 7.24-7.23 (m, 1H), 7.01-6.99 (m, 1H), 6.94-6.91 (m, 1H), 6.57-6.53 (m, 2H), 5.93-5.85 (m, 1H), 5.74 (d, 1H), 4.64-4.59 (m, 1H), 3.35-3.28 (m, 1H), 3.13-3.08 (m, 1H), 2.65 (s, 3H), 1.39 (d, 3H); Chiral analytical SFC: RT=3.04 min, Column: Chiralpak AD-3, (4.6×150 mm, 3 m), 70% CO₂/(0.5% DEA in Ethanol), Flow=3.0 g/min.

(2S)-N-(1-(6,7-Difluoro-1-oxo-1,2-dihydroisoquinolin-4-yl)ethyl)-N-methylindoline-2-carboxamide—Diastereoisomer II (Compound 31), LCMS: m/z found 384.3 [M+H]⁺, RT=2.77 min (Method A); ¹H NMR (400 MHz, DMSO-d₆): δ 11.65 (bs, 1H), 8.14-8.09 (m, 1H), 7.28-7.22 (m, 2H), 6.96-6.92 (m, 2H), 6.60-6.53 (m, 2H), 5.89-5.83 (m, 1H), 5.75 (s, 1H), 4.65-4.61 (m, 1H), 3.28-3.20 (m, 1H), 2.79-2.74 (m, 1H), 2.61 (s, 3H), 1.41 (d, 3H); Chiral analytical SFC: RT=4.72 min, Column: Chiralpak AD-3, (4.6×150 mm, 3 μm), 70% CO₂/(0.5% DEA in Ethanol), Flow=3.0 g/min.

### N-(1-(6,7-Difluoro-1-oxo-1,2-dihydroisoquinolin-4-yl)ethyl)-5-fluoro-N-methylindoline-2-carboxamide (Compounds 61, 62, 63, & 64)

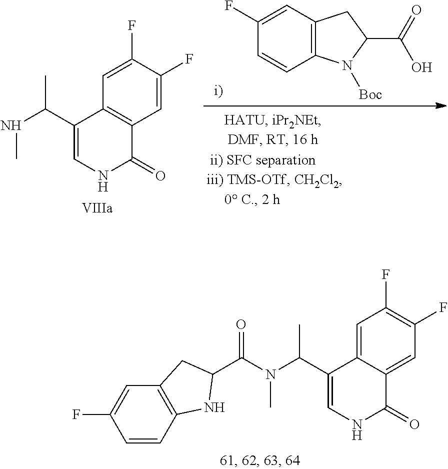 [SMILES: CNC(C)c1c[nH]c(=O)c2cc(F)c(F)cc12 ; CC(C)(C)OC(=O)N1c2ccc(F)cc2CC1C(=O)O ; CC(c1c[nH]c(=O)c2cc(F)c(F)cc12)N(C)C(=O)C1Cc2cc(F)ccc2N1]

tert-Butyl 2-((1-(6,7-difluoro-1-oxo-1,2-dihydroisoquinolin-4-yl)ethyl)(methyl)carbamoyl)-5-fluoroindoline-1-carboxylate as a mixture of four stereoisomers was synthesized in a similar manner as described above from 1H-6,7-difluoro-4-(1-(methylamino)ethyl)isoquinolin-1(2H)-one (VIIIa) and racemic 1-(tert-butoxycarbonyl)-5-fluoroindoline-2-carboxylic acid. The stereoisomers were subsequently separated by chiral SFC, Column: Chiralcel OD (250×30 mm, 5 μm), 80% CO₂/Methanol, Flow rate 110 g/min to provide pure first (RT=2.79 min) and last (RT=4.82 min) eluting stereoisomers. The remaining two stereoisomers (RT=3.44 min and 3.89 min) were separated by a second chiral SFC method: Column: Chiralpak IG (250×30 mm, 5 μm), 80% CO₂/Isopropanol, Flow rate 100 g/min. Each isolated stereoisomer of tert-butyl 2-((1-(6,7-difluoro-1-oxo-1,2-dihydroisoquinolin-4-yl)ethyl)(methyl)carbamoyl)-5-fluoroindoline-1-carboxylate was converted to a single stereoisomer of N-(1-(6,7-difluoro-1-oxo-1,2-dihydroisoquinolin-4-yl)ethyl)-5-fluoro-N-methylindoline-2-carboxamide by treatment with TMSOTf in a similar manner as described above.

N-(1-(6,7-Difluoro-1-oxo-1,2-dihydroisoquinolin-4-yl)ethyl)-5-fluoro-N-methylindoline-2-carboxamide—Stereoisomer IA (Compound 61), LCMS: m/z found 402.2 [M+H]⁺, RT=2.91 min (Method A); ¹H NMR (400 MHz, DMSO-d₆): δ 11.71 (bs, 1H), 8.14-8.09 (m, 1H), 7.27-7.22 (m, 2H), 6.81-6.73 (m, 2H), 6.57-6.53 (m, 1H), 5.88-5.83 (m, 1H), 5.65 (s, 1H), 4.67-4.63 (m, 1H), 3.31-3.20 (m, 1H), 2.77 (dd, 1H), 2.60 (s, 3H), 1.40 (d, 3H); Chiral analytical SFC: RT=3.40 min, Column: Chiralpak IC-3, (4.6×150 mm, 3 μm), 70% CO₂/(0.5% DEA in Methanol), Flow=3.0 g/min.

N-(1-(6,7-Difluoro-1-oxo-1,2-dihydroisoquinolin-4-yl)ethyl)-5-fluoro-N-methylindoline-2-carboxamide—Stereoisomer IIA (Compound 62, enantiomer of Compound 61), LCMS: m/z found 402.2 [M+H]⁺, RT=2.91 min (Method A); ¹H NMR (400 MHz, DMSO-d₆): δ 11.71 (bs, 1H), 8.14-8.09 (m, 1H), 7.27-7.22 (m, 2H), 6.81-6.73 (m, 2H), 6.57-6.53 (m, 1H), 5.88-5.83 (m, 1H), 5.65 (s, 1H), 4.67-4.63 (m, 1H), 3.31-3.20 (m, 1H), 2.77 (dd, 1H), 2.60 (s, 3H), 1.40 (d, 3H); Chiral analytical SFC: RT=3.92 min, Column: Chiralpak IC-3, (4.6×150 mm, 3 μm), 70% CO₂/(0.5% DEA in Methanol), Flow=3.0 g/min.

N-(1-(6,7-Difluoro-1-oxo-1,2-dihydroisoquinolin-4-yl)ethyl)-5-fluoro-N-methylindoline-2-carboxamide—Stereoisomer IB (Compound 63), LCMS: m/z found 402.2 [M+H]⁺, RT=2.91 min (Method A); ¹H NMR (400 MHz, DMSO-d₆): δ 11.71 (bs, 1H), 8.13-8.09 (m, 1H), 7.54-7.49 (m, 1H), 7.23 (s, 1H), 6.87 (dd, 1H), 6.76-6.71 (m, 1H), 6.53-6.49 (m, 1H), 5.88-5.83 (m, 1H), 5.63 (s, 1H), 4.65-4.64 (m, 1H), 3.34-3.29 (m, 1H), 3.28-3.15 (m, 1H), 2.64 (s, 3H), 1.38 (d, 3H); Chiral analytical SFC: RT=2.56 min, Column: Chiralcel OD-3, (4.6×150 mm, 3 μm), 70% CO₂/(0.5% DEA in Methanol), Flow=3.0 g/min.

N-(1-(6,7-Difluoro-1-oxo-1,2-dihydroisoquinolin-4-yl)ethyl)-5-fluoro-N-methylindoline-2-carboxamide—Stereoisomer IIB (Compound 64, enantiomer of Compound 63), LCMS: m/z found 402.2 [M+H]⁺, RT=2.91 min (Method A); ¹H NMR (400 MHz, DMSO-d₆): δ 11.71 (br s, 1H), 8.13-8.09 (m, 1H), 7.54-7.49 (m, 1H), 7.23 (s, 1H), 6.87 (dd, 1H), 6.76-6.71 (m, 1H), 6.53-6.49 (m, 1H), 5.88-5.83 (m, 1H), 5.63 (s, 1H), 4.65-4.64 (m, 1H), 3.34-3.28 (m, 1H), 3.17-3.11 (m, 1H), 2.64 (s, 3H), 1.38 (d, 3H); Chiral analytical SFC: RT=3.48 min, Column: Chiralcel OD-3, (4.6×150 mm, 3 μm), 70% CO₂/(0.5% DEA in Methanol), Flow=3.0 g/min.

### N-(1-(6,7-Difluoro-1-oxo-1,2-dihydroisoquinolin-4-yl)ethyl)-4,6-difluoro-N-methylindoline-2-carboxamide (Compounds 98, 99, 100, & 101)

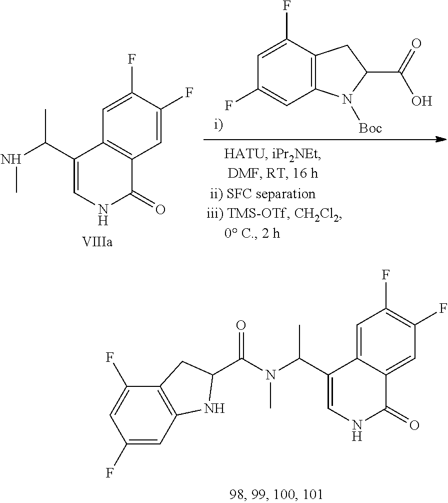

tert-Butyl 2-((1-(6,7-difluoro-1-oxo-1,2-dihydroisoquinolin-4-yl)ethyl)(methyl)carbamoyl)-4,6-difluoroindoline-1-carboxylate as a mixture of four stereoisomers was synthesized in a similar manner as described above from 1H-6,7-difluoro-4-(1-(methylamino)ethyl)isoquinolin-1(2H)-one (VIIIa) and racemic 1-(tert-butoxycarbonyl)-4,6-difluoroindoline-2-carboxylic acid. The stereoisomers were subsequently separated by chiral SFC, Column: Chiralpak IC (250×30 mm, 5 μm), 80% CO₂/Methanol, Flow rate 100 g/min to isolate pure last (RT=3.96 min) eluting stereoisomer. The remaining three stereoisomers were separated by a second chiral SFC method: Column: Chiralcel OD-H (250×30 mm, 5 μm), 80% CO₂/Methanol, Flow rate 120 g/min (RT=1.81, 2.11, and 2.82 min). Each isolated stereoisomer of tert-butyl 2-((1-(6,7-difluoro-1-oxo-1,2-dihydroisoquinolin-4-yl)ethyl)(methyl)carbamoyl)-4,6-difluoroindoline-1-carboxylate was converted to a single stereoisomer of N-(1-(6,7-difluoro-1-oxo-1,2-dihydroisoquinolin-4-yl)ethyl)-4,6-difluoro-N-methylindoline-2-carboxamide by treatment with TMSOTf in a similar manner as described above.

N-(1-(6,7-Difluoro-1-oxo-1,2-dihydroisoquinolin-4-yl)ethyl)-4,6-difluoro-N-methylindoline-2-carboxamide—Stereoisomer IA (Compound 98), LCMS: m/z found 420.2 [M+H]⁺, RT=3.88 (Method A); ¹H NMR (400 MHz, DMSO-d₆): 11.64 (bs, 1H), 8.12 (t, 1H), 7.25-7.20 (m, 2H), 6.50 (s, 1H), 6.25-6.23 (m, 2H), 5.88-5.82 (m, 1H), 4.80-4.76 (m, 1H), 3.24-3.17 (m, 1H), 2.67-2.61 (m, 1H), 2.60 (s, 3H), 1.41 (d, 3H); Chiral analytical SFC: RT=3.53 min, Column: Chiralcel OD-3, (4.6×150 mm, 3 μm), 75% CO₂/Methanol, Flow=3.0 g/min.

N-(1-(6,7-Difluoro-1-oxo-1,2-dihydroisoquinolin-4-yl)ethyl)-4,6-difluoro-N-methylindoline-2-carboxamide—Stereoisomer IIA (Compound 99, enantiomer of Compound 98), LCMS: m/z found 420.2 [M+H]⁺, RT=3.88 min (Method A); ¹H NMR (400 MHz, DMSO-d₆): δ 11.64 (bs, 1H), 8.12 (t, 1H), 7.25-7.20 (m, 2H), 6.50 (s, 1H), 6.25-6.23 (m, 2H), 5.88-5.82 (m, 1H), 4.80-4.76 (m, 1H), 3.24-3.17 (m, 1H), 2.67-2.61 (m, 1H), 2.60 (s, 3H), 1.41 (d, 3H); Chiral analytical SFC: RT=4.17 min, Column: Chiralcel OD-3, (4.6×150 mm, 3 μm), 75% CO₂/Methanol, Flow=3.0 g/min.

N-(1-(6,7-Difluoro-1-oxo-1,2-dihydroisoquinolin-4-yl)ethyl)-4,6-difluoro-N-methylindoline-2-carboxamide—Stereoisomer IB (Compound 100), LCMS: m/z found 420.2 [M+H]⁺, RT=4.05 min (Method A); ¹H NMR (400 MHz, DMSO-d₆): δ 11.65 (bs, 1H), 8.11 (t, 1H), 7.52-7.47 (m, 1H), 7.24 (bs, 1H), 6.50 (s, 1H), 6.24-6.15 (m, 2H), 5.91-5.86 (m, 1H), 4.80-4.77 (m, 1H), 3.33-3.29 (m, 1H), 3.10-3.05 (m, 1H), 2.64 (s, 3H), 1.39 (d, 3H); Chiral analytical SFC: RT=1.48 min, Column: Chiralcel OD-3, (4.6×150 mm, 3 μm), 60% CO₂/Methanol, Flow=3.0 g/min.

N-(1-(6,7-Difluoro-1-oxo-1,2-dihydroisoquinolin-4-yl)ethyl)-4,6-difluoro-N-methylindoline-2-carboxamide—Stereoisomer IIB (Compound 101, enantiomer of Compound 100), LCMS: m/z found 420.2 [M+H]⁺, RT=4.05 min (Method A); ¹H NMR (400 MHz, DMSO-d₆): δ 11.65 (bs, 1H), 8.11 (t, 1H), 7.52-7.47 (m, 1H), 7.24 (bs, 1H), 6.50 (s, 1H), 6.24-6.15 (m, 2H), 5.91-5.86 (m, 1H), 4.80-4.77 (m, 1H), 3.33-3.29 (m, 1H), 3.10-3.05 (m, 1H), 2.64 (s, 3H), 1.39 (d, 3H); Chiral analytical SFC: RT=2.81 min, Column Chiralcel OD-3, (4.6×150 mm, 3 μm), 60% CO₂/Methanol, Flow=3.0 g/min.

### N-(1-(6,7-Difluoro-1-oxo-1,2-dihydroisoquinolin-4-yl)ethyl)-N-methyl-1H-benzo[d]imidazole-2-carboxamide (Compounds 23 & 24)

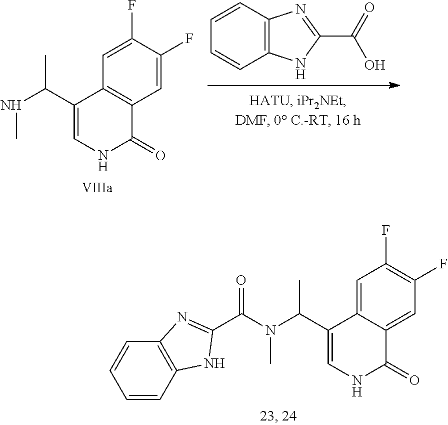

Racemic N-(1-(6,7-difluoro-1-oxo-1,2-dihydroisoquinolin-4-yl)ethyl)-N-methyl-1H-benzo[d]imidazole-2-carboxamide was synthesized in a similar manner as described above from 1H-6,7-difluoro-4-(1-(methylamino)ethyl)isoquinolin-1(2H)-one (VIIIa) and 1H-benzo[d]imidazole-2-carboxylic acid. The enantiomers were subsequently separated by chiral SFC, Column: Chiralcel OX-H (250×30 mm, 5 μm) 50% CO₂/MeOH, Flow rate 70 g/min.

N-(1-(6,7-Difluoro-1-oxo-1,2-dihydroisoquinolin-4-yl)ethyl)-N-methyl-1H-benzo[d]imidazole-2-carboxamide—Enantiomer I (Compound 23), LCMS: m/z found 383.3 [M+H]⁺, RT=3.30 min (Method A); ¹H NMR (400 MHz, DMSO-d₆, 90° C.); δ 13.27 (bs, 1H), 11.72 (bs, 1H), 8.15-8.11 (m, 1H), 7.70-7.59 (m, 3H), 7.35-7.25 (m, 3H), 6.18-6.13 (m, 1H), 3.20 (s, 3H), 1.57 (d, 3H); Chiral analytical SFC: RT=2.53 min, Column: Chiralpak IG-3, (4.6×150 mm, 3 m), 60% CO₂/MeOH, Flow=3.0 g/min.

N-(1-(6,7-Difluoro-1-oxo-1,2-dihydroisoquinolin-4-yl)ethyl)-N-methyl-1H-benzo[d]imidazole-2-carboxamide—Enantiomer II (Compound 24), LCMS: m/z found 383.3 [M+H]⁺, RT=3.30 min (Method A); ¹H NMR (400 MHz, DMSO-d₆, 90° C.); δ 13.27 (bs, 1H), 11.72 (bs, 1H), 8.15-8.11 (m, 1H), 7.70-7.59 (m, 3H), 7.35-7.25 (m, 3H), 6.18-6.13 (m, 1H), 3.20 (s, 3H), 1.57 (d, 3H); Chiral analytical SFC: RT=4.21 min, Column: Chiralpak IG-3, (4.6×150 mm, 3 μm), 60% CO₂/MeOH, Flow=3.0 g/min.

### N-(1-(6,7-Difluoro-1-oxo-1,2-dihydroisoquinolin-4-yl)ethyl)-N-methylbenzofuran-2-carboxamide (Compounds 25 & 26)

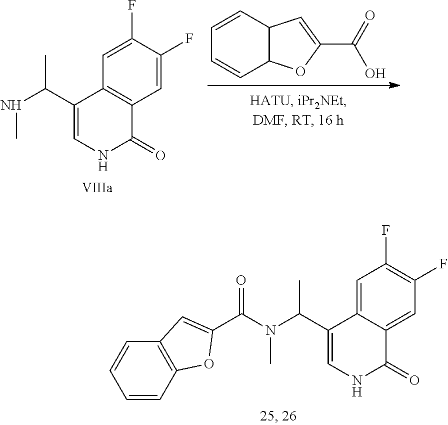

Racemic N-(1-(6,7-difluoro-1-oxo-1,2-dihydroisoquinolin-4-yl)ethyl)-N-methylbenzofuran-2-carboxamide was synthesized in a similar manner as described above from 1H-6,7-difluoro-4-(1-(methylamino)ethyl)isoquinolin-1(2H)-one (VIIIa) and benzofuran-2-carboxylic acid. The enantiomers were subsequently separated by chiral SFC, Column: Chiralpak IC (250×30 mm, 5 μm) 65% CO₂/MeOH, Flow rate 100 g/min.

N-(1-(6,7-Difluoro-1-oxo-1,2-dihydroisoquinolin-4-yl)ethyl)-N-methylbenzofuran-2-carboxamide—Enantiomer I (Compound 25), LCMS: m/z found 383.3 [M+H]⁺, RT=4.02 min (Method A); ¹H NMR (400 MHz, DMSO-d₆); δ 11.71 (bs, 1H), 8.16-8.11 (m, 1H), 7.75-7.73 (m, 1H), 7.67-7.65 (m, 1H), 7.59-7.53 (m, 1H), 7.49 (s, 1H), 7.47-7.43 (m, 1H), 7.35-7.31 (m, 2H), 6.06-6.02 (m, 1H), 2.88 (s, 3H), 1.55 (d, 3H); Chiral analytical SFC: RT=3.44 min, Column: Chiralpak IC-3, (4.6×150 mm, 3 μm), 65% CO₂/MeOH, Flow=3.0 g/min.

N-(1-(6,7-Difluoro-1-oxo-1,2-dihydroisoquinolin-4-yl)ethyl)-N-methylbenzofuran-2-carboxamide—Enantiomer II (Compound 26), LCMS: m/z found 383.3 [M+H]⁺, RT=4.02 min (Method A); ¹H NMR (400 MHz, DMSO-d₆); δ 11.71 (bs, 1H), 8.16-8.11 (m, 1H), 7.75-7.73 (m, 1H), 7.67-7.65 (m, 1H), 7.59-7.53 (m, 1H), 7.49 (s, 1H), 7.47-7.43 (m, 1H), 7.35-7.31 (m, 2H), 6.06-6.02 (m, 1H), 2.88 (s, 3H), 1.55 (d, 3H); Chiral analytical SFC: RT=4.26 min, Column: Chiralpak IC-3, (4.6×150 mm, 3 μm), 65% CO₂/MeOH, Flow=3.0 g/min.

### N-(1-(6,7-Difluoro-1-oxo-1,2-dihydroisoquinolin-4-yl)ethyl)-N-methylimidazo[1,2-a]pyridine-2-carboxamide (Compounds 45 & 46)

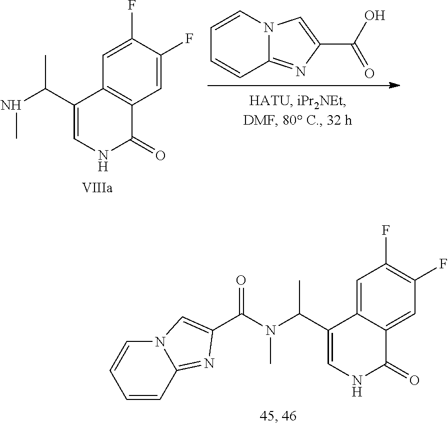

Racemic N-(1-(6,7-difluoro-1-oxo-1,2-dihydroisoquinolin-4-yl)ethyl)-N-methylimidazo[1,2-a]pyridine-2-carboxamide was synthesized in a similar manner as described above, except at 80° C. instead of room temperature, from 1H-6,7-difluoro-4-(1-(methylamino)ethyl)isoquinolin-1(2H)-one (VIIIa) and imidazo[1,2-a]pyridine-2-carboxylic acid. The enantiomers were subsequently separated by chiral SFC, Column: Chiralpak AD-H (250×30 mm, 5 μm) 65% CO₂/MeOH, Flow rate 100 g/min.

N-(1-(6,7-Difluoro-1-oxo-1,2-dihydroisoquinolin-4-yl)ethyl)-N-methylimidazo[1,2-a]pyridine-2-carboxamide—Enantiomer I (Compound 45), LCMS: m/z found 383.2 [M+H]⁺, RT=1.89 min (Method A); ¹H NMR (400 MHz, DMSO-d₆, 90° C.): δ 11.36 (bs, 1H), 8.55 (d, 1H), 8.35 (s, 1H), 8.11-8.06 (m, 1H), 7.80 (bs, 1H), 7.55 (d, 1H), 7.31-7.27 (m, 1H), 7.22 (s, 1H), 6.96-6.92 (m, 1H), 6.36-6.31 (m, 1H), 2.89 (s, 3H), 1.52 (d, 3H); Chiral analytical SFC: RT=1.55 min, Column: Chiralpak AD-3, (4.6×150 mm, 3 μm), 60% CO₂/(0.5% Isopropyl amine in iso-propanol), Flow=3.0 g/min.

N-(1-(6,7-Difluoro-1-oxo-1,2-dihydroisoquinolin-4-yl)ethyl)-N-methylimidazo[1,2-a]pyridine-2-carboxamide—Enantiomer II (Compound 46), LCMS: m/z found 383.2 [M+H]⁺, RT=1.89 min (Method A); ¹H NMR (400 MHz, DMSO-d₆, 90° C.): δ 11.36 (bs, 1H), 8.55 (d, 1H), 8.35 (s, 1H), 8.11-8.06 (m, 1H), 7.80 (bs, 1H), 7.55 (d, 1H), 7.31-7.27 (m, 1H), 7.22 (s, 1H), 6.96-6.92 (m, 1H), 6.36-6.31 (m, 1H), 2.89 (s, 3H), 1.52 (d, 3H), 1.55 (d, 3H); Chiral analytical SFC: RT=2.27 min, Column: Chiralpak AD-3, (4.6×150 mm, 3 μm), 60% CO₂/(0.5% Isopropyl amine in iso-propanol), Flow=3.0 g/min.

### N-(1-(6,7-Difluoro-1-oxo-1,2-dihydroisoquinolin-4-yl)ethyl)-N-methyl-1,4,5,6-tetrahydro cyclopenta[b]pyrrole-2-carboxamide (Compounds 47 & 48)

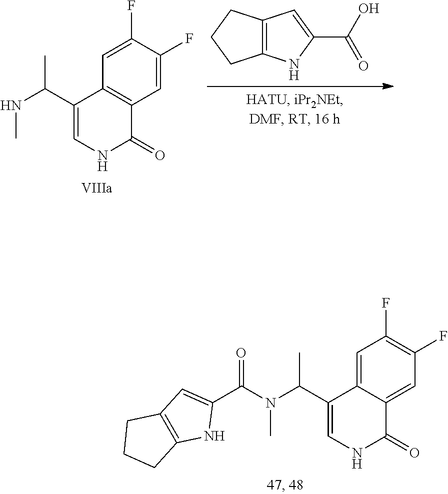

Racemic N-(1-(6,7-difluoro-1-oxo-1,2-dihydroisoquinolin-4-yl)ethyl)-N-methyl-1,4,5,6-tetrahydro cyclopenta[b]pyrrole-2-carboxamide was synthesized in a similar manner as described above, from 1H-6,7-difluoro-4-(1-(methylamino)ethyl)isoquinolin-1(2H)-one (VIIIa) and 1,4,5,6-tetrahydrocyclopenta[b]pyrrole-2-carboxylic acid. The enantiomers were subsequently separated by chiral SFC, Column: Chiralpak IC (250×30 mm, 5 m), 70% CO₂/(30 mM methanolic ammonia in methanol), Flow rate 90 g/min.

N-(1-(6,7-Difluoro-1-oxo-1,2-dihydroisoquinolin-4-yl)ethyl)-N-methyl-1,4,5,6-tetrahydro cyclopenta[b]pyrrole-2-carboxamide—Enantiomer I (Compound 47), LCMS: m/z found 372.2 [M+H]⁺, RT=3.90 min (Method A); ¹H NMR (400 MHz, DMSO-d₆): δ 11.64 (bs, 1H), 11.23 (bs, 1H), 8.13-8.08 (m, 1H), 7.63-7.61 (m, 1H), 7.25 (s, 1H), 6.29 (s, 1H), 6.06-6.03 (m, 1H), 2.79 (s, 3H), 2.67-2.60 (m, 2H), 2.54-2.49 (m, 2H), 2.34-2.29 (m, 2H), 1.45 (d, 3H); Chiral analytical SFC: RT=6.12 min, Column: Chiralpak IC-3, (4.6×150 mm, 3 μm), 75% CO₂/Methanol, Flow=3.0 g/min.

N-(1-(6,7-Difluoro-1-oxo-1,2-dihydroisoquinolin-4-yl)ethyl)-N-methyl-1,4,5,6-tetrahydro cyclopenta[b]pyrrole-2-carboxamide—Enantiomer II (Compound 48), LCMS: m/z found 372.2 [M+H]⁺, RT=3.90 min (Method A); ¹H NMR (400 MHz, DMSO-d₆): δ 11.64 (bs, 1H), 11.23 (bs, 1H), 8.13-8.08 (m, 1H), 7.63-7.61 (m, 1H), 7.25 (s, 1H), 6.29 (s, 1H), 6.06-6.03 (m, 1H), 2.79 (s, 3H), 2.67-2.60 (m, 2H), 2.54-2.49 (m, 2H), 2.34-2.29 (m, 2H), 1.45 (d, 3H); Chiral analytical SFC: RT=7.36 min, Column: Chiralpak IC-3, (4.6×150 mm, 3 μm), 75% CO₂/Methanol, Flow=3.0 g/min.

### N-(1-(6,7-Difluoro-1-oxo-1,2-dihydroisoquinolin-4-yl)ethyl)-5,6-difluoro-N-methyl-1H-benzo[d]imidazole-2-carboxamide (Compounds 49 & 50)

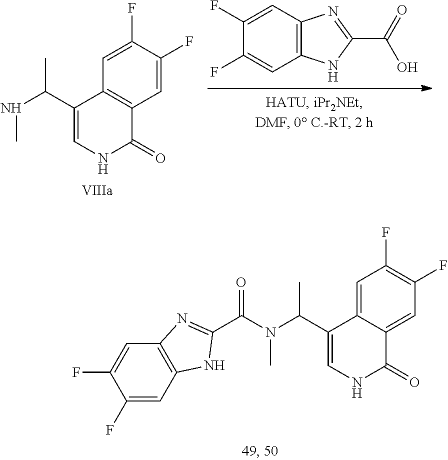

Racemic N-(1-(6,7-difluoro-1-oxo-1,2-dihydroisoquinolin-4-yl)ethyl)-5,6-difluoro-N-methyl-1H-benzo[d]imidazole-2-carboxamide was synthesized in a similar manner as described above, from 1H-6,7-difluoro-4-(1-(methylamino)ethyl)isoquinolin-1(2H)-one (VIIIa) and 5,6-difluoro-1H-benzo[d]imidazole-2-carboxylic acid. The enantiomers were subsequently separated by chiral SFC, Column: DAICEL DCPAK (250×30 mm, 5 μm), 75% CO₂/(0.4% (7 M methanolic ammonia) in isopropanol), Flow rate 90 g/min.

N-(1-(6,7-Difluoro-1-oxo-1,2-dihydroisoquinolin-4-yl)ethyl)-5,6-difluoro-N-methyl-1H-benzo[d]imidazole-2-carboxamide—Enantiomer I (Compound 49), LCMS: m/z found 419.2 [M+H]⁺, RT=3.92 min (Method A); ¹H NMR (400 MHz, DMSO-d₆, 90° C.): δ 13.20 (bs, 1H), 11.40 (bs, 1H), 8.12-8.07 (m, 1H), 7.72-7.53 (m, 3H), 7.27 (s, 1H), 6.15-6.10 (m, 1H), 3.13 (s, 3H), 1.56 (d, 3H); Chiral analytical SFC: RT=2.75 min, Column: Chiralpak IC-3, (4.6×150 mm, 3 μm), 80% CO₂/(0.5% isopropylamine in isopropanol), Flow=3.0 g/min.

N-(1-(6,7-Difluoro-1-oxo-1,2-dihydroisoquinolin-4-yl)ethyl)-5,6-difluoro-N-methyl-1H-benzo[d]imidazole-2-carboxamide—Enantiomer II (Compound 50), LCMS: m/z found 419.2 [M+H]⁺, RT=3.92 min (Method A); ¹H NMR (400 MHz, DMSO-d₆, 90° C.): δ 13.20 (bs, 1H), 11.40 (bs, 1H), 8.12-8.07 (m, 1H), 7.72-7.53 (m, 3H), 7.27 (s, 1H), 6.15-6.10 (m, 1H), 3.13 (s, 3H), 1.56 (d, 3H); Chiral analytical SFC: RT=3.43 min, Column: Chiralpak IC-3, (4.6×150 mm, 3 μm), 80% CO₂/(0.5% isopropylamine in isopropanol), Flow=3.0 g/min.

### N-(1-(6,7-Difluoro-1-oxo-1,2-dihydroisoquinolin-4-yl)ethyl)-N-methylindolizine-2-carboxamide (Compounds 69 & 70)

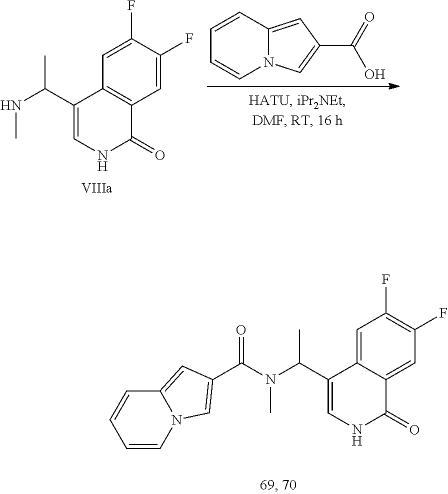

Racemic N-(1-(6,7-difluoro-1-oxo-1,2-dihydroisoquinolin-4-yl)ethyl)-N-methylindolizine-2-carboxamide was synthesized in a similar manner as described above, from 1H-6,7-difluoro-4-(1-(methylamino)ethyl)isoquinolin-1(2H)-one (VIIIa) and indolizine-2-carboxylic acid. The enantiomers were subsequently separated by chiral SFC, Column: Chiralpak IC (250×30 mm, 5 μm), 75% CO₂/Methanol, Flow rate 120 g/min.

N-(1-(6,7-Difluoro-1-oxo-1,2-dihydroisoquinolin-4-yl)ethyl)-N-methylindolizine-2-carboxamide—Enantiomer I (Compound 69), LCMS: m/z found 382.2 [M+H]⁺, RT=3.55 min (Method A); ¹H NMR (400 MHz, DMSO-d₆): δ 11.65 (bs, 1H), 8.25-8.21 (m, 1H) 8.12 (t, 1H), 7.87 (s, 1H), 7.69-7.58 (m, 1H), 7.41 (d, 1H), 7.28 (s, 1H), 6.73 (t, 1H), 6.65-6.54 (m, 2H), 6.18-6.07 (m, 1H), 2.78 (s, 3H), 1.51 (d, 3H); Chiral analytical SFC: RT=4.00 min, Column: Chiralpak IC-3, (4.6×150 mm, 3 μm), 60% CO₂/Methanol, Flow=3.0 g/min.

N-(1-(6,7-Difluoro-1-oxo-1,2-dihydroisoquinolin-4-yl)ethyl)-N-methylindolizine-2-carboxamide—Enantiomer II (Compound 70), LCMS: m/z found 382.2 [M+H]⁺, RT=3.55 min (Method A); ¹H NMR (400 MHz, DMSO-d₆): δ 11.67 (bs, 1H), 8.25-8.21 (m, 1H) 8.12 (t, 1H), 7.88 (s, 1H), 7.69-7.58 (m, 1H), 7.41 (d, 1H), 7.28 (s, 1H), 6.73 (t, 1H), 6.65-6.54 (m, 2H), 6.18-6.07 (m, 1H), 2.79 (s, 3H), 1.51 (d, 3H); Chiral analytical SFC: RT=5.25 min, Column: Chiralpak IC-3, (4.6×150 mm, 3 μm), 60% CO₂/Methanol, Flow=3.0 g/min.

### N-(1-(6,7-Difluoro-1-oxo-1,2-dihydroisoquinolin-4-yl)ethyl)-N-isobutylindolizine-2-carboxamide (Compound 116)

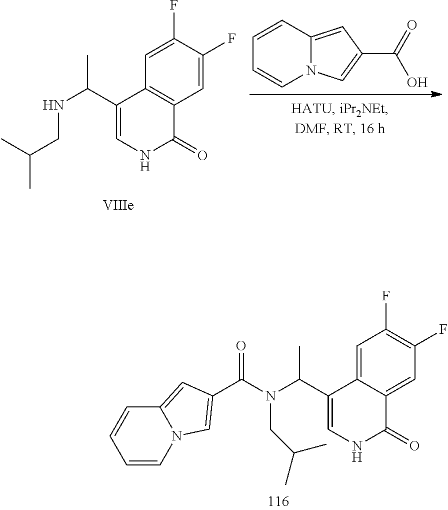

Racemic N-(1-(6,7-difluoro-1-oxo-1,2-dihydroisoquinolin-4-yl)ethyl)-N-isobutylindolizine-2-carboxamide was synthesized in a similar manner as described above, from 6,7-difluoro-4-(1-(isobutylamino)ethyl)isoquinolin-1(2H)-one (VIIIc) and indolizine-2-carboxylic acid. LCMS: m/z found 424.3 [M+H]⁺, RT=6.69 min (Method A); ¹H NMR (400 MHz, DMSO-d₆): δ 11.67 (br s, 1H), 8.27-7.35 (m, 6H), 6.75-6.61 (m, 3H), 6.005 (m, 1H), 3.09 (m, 2H), 1.63 (br s, 4H), 0.49 (br s, 6H).

### 8-Chloro-N-(1-(6,7-difluoro-1-oxo-1,2-dihydroisoquinolin-4-yl)ethyl)-N-methylindolizine-2-carboxamide (Compounds 77 & 78)

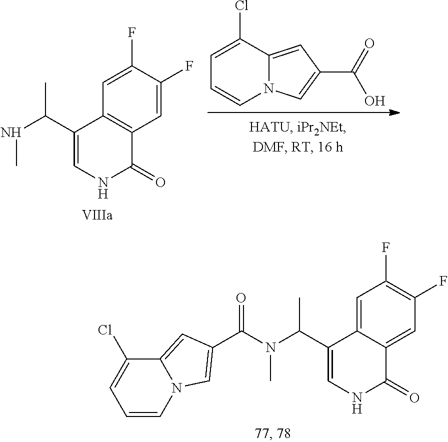

Racemic 8-chloro-N-(1-(6,7-difluoro-1-oxo-1,2-dihydroisoquinolin-4-yl)ethyl)-N-methylindolizine-2-carboxamide was synthesized in a similar manner as described above, from 1H-6,7-difluoro-4-(1-(methylamino)ethyl)isoquinolin-1(2H)-one (VIIIa) and 8-chloroindolizine-2-carboxylic acid. The enantiomers were subsequently separated by chiral SFC, Column: Chiralpak IC (250×30 mm, 5 μm), 60% CO₂/Methanol, Flow rate 100 g/min.

8-Chloro-N-(1-(6,7-difluoro-1-oxo-1,2-dihydroisoquinolin-4-yl)ethyl)-N-methylindolizine-2-carboxamide—Enantiomer I (Compound 77), LCMS: m/z found 416.2/418.2 [M+H]⁺, RT=4.08 min (Method A); ¹H NMR (400 MHz, DMSO-d₆): δ 11.68 (bs, 1H), 8.25 (d, 1H), 8.13 (t, 1H), 8.03 (s, 1H), 7.63 (bs, 1H), 7.29 (s, 1H), 6.95 (d, 1H), 6.69-6.62 (m, 2H), 6.07 (s, 1H), 2.79 (s, 3H), 1.52 (s, 3H); Chiral analytical SFC: RT=4.19 min, Column: Chiralpak IC-3, (4.6×150 mm, 3 μm), 60% CO₂/Methanol, Flow=3.0 g/min. 8-Chloro-N-(1-(6,7-difluoro-1-oxo-1,2-dihydroisoquinolin-4-yl)ethyl)-N-methylindolizine-2-carboxamide—Enantiomer II (Compound 78), LCMS: m/z found 416.2/418.2 [M+H]⁺, RT=4.08 min (Method A); ¹H NMR (400 MHz, DMSO-d₆): δ 11.68 (bs, 1H), 8.25 (d, 1H), 8.13 (t, 1H), 8.03 (s, 1H), 7.63 (bs, 1H), 7.29 (s, 1H), 6.95 (d, 1H), 6.69-6.62 (m, 2H), 6.07 (s, 1H), 2.79 (s, 3H), 1.52 (s, 3H); Chiral analytical SFC: RT=5.28 min, Column: Chiralpak IC-3, (4.6×150 mm, 3 μm), 60% CO₂/Methanol, Flow=3.0 g/min.

### N-(1-(6,7-Difluoro-1-oxo-1,2-dihydroisoquinolin-4-yl)ethyl)-8-fluoro-N-methylindolizine-2-carboxamide (Compounds 79 & 80)

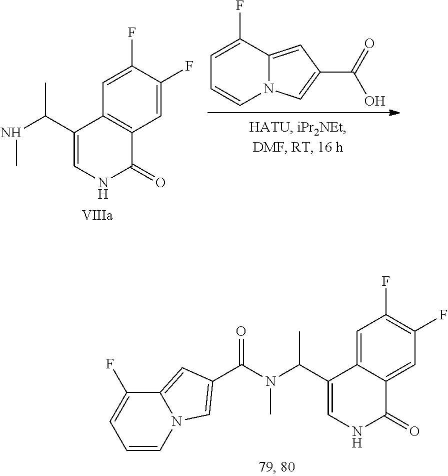

Racemic N-(1-(6,7-difluoro-1-oxo-1,2-dihydroisoquinolin-4-yl)ethyl)-8-fluoro-N-methylindolizine-2-carboxamide was synthesized in a similar manner as described above, from 1H-6,7-difluoro-4-(1-(methylamino)ethyl)isoquinolin-1(2H)-one (VIIIa) and 8-fluoroindolizine-2-carboxylic acid. The enantiomers were subsequently separated by chiral SFC, Column: Chiralpak IC (250×30 mm, 5 μm), 60% CO₂/Methanol, Flow rate 100 g/min.

N-(1-(6,7-Difluoro-1-oxo-1,2-dihydroisoquinolin-4-yl)ethyl)-8-fluoro-N-methylindolizine-2-carboxamide—Enantiomer I (Compound 79), LCMS: m/z found 400.2 [M+H]⁺, RT=3.76 min (Method A); ¹H NMR (400 MHz, DMSO-d₆): δ 11.66 (bs, 1H), 8.15-8.04 (m, 3H), 7.62 (bs, 1H), 7.28 (s, 1H), 6.73 (bs, 1H), 6.63-6.61 (d, 2H), 6.07 (s, 1H), 2.79 (s, 3H), 1.52 (s, 3H); Chiral analytical SFC: RT=3.05 min, Column: Chiralpak IC-3, (4.6×150 mm, 3 μm), 60% CO₂/Methanol, Flow=3.0 g/min.

N-(1-(6,7-Difluoro-1-oxo-1,2-dihydroisoquinolin-4-yl)ethyl)-8-fluoro-N-methylindolizine-2-carboxamide—Enantiomer II (Compound 80), LCMS: m/z found 400.2 [M+H]⁺, RT=3.76 min (Method A); ¹H NMR (400 MHz, DMSO-d₆): δ 11.66 (bs, 1H), 8.15-8.04 (m, 3H), 7.62 (bs, 1H), 7.28 (s, 1H), 6.73 (bs, 1H), 6.63-6.61 (d, 2H), 6.07 (s, 1H), 2.79 (s, 3H), 1.52 (s, 3H); Chiral analytical SFC: RT=3.82 min, Column: Chiralpak IC-3, (4.6×150 mm, 3 μm), 60% CO₂/Methanol, Flow=3.0 g/min.

### 7-Chloro-N-(1-(6,7-difluoro-1-oxo-1,2-dihydroisoquinolin-4-yl)ethyl)-N-methylindolizine-2-carboxamide (Compounds 96 & 97)

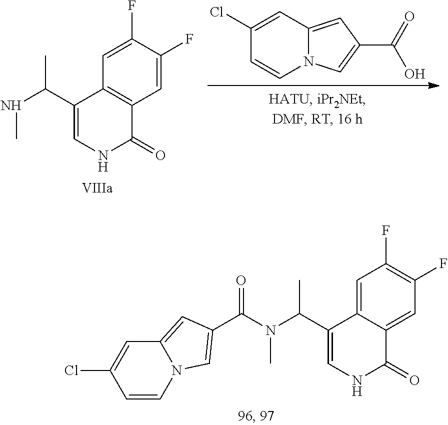

Racemic 7-chloro-N-(1-(6,7-difluoro-1-oxo-1,2-dihydroisoquinolin-4-yl)ethyl)-N-methylindolizine-2-carboxamide was synthesized in a similar manner as described above from 1H-6,7-difluoro-4-(1-(methylamino)ethyl)isoquinolin-1(2H)-one (VIIIa) and 7-chloroindolizine-2-carboxylic acid. The enantiomers were subsequently separated by chiral SFC, Column: Chiralpak IC (250×30 mm, 5 μm), 60% CO₂/Methanol, Flow rate 110 g/min.

7-Chloro-N-(1-(6,7-difluoro-1-oxo-1,2-dihydroisoquinolin-4-yl)ethyl)-N-methylindolizine-2-carboxamide—Enantiomer I (Compound 96), LCMS: m/z found 416.2/418.2 [M+H]⁺, RT=4.16 min (Method A); ¹H NMR (400 MHz, DMSO-d₆): δ 11.55 (bs, 1H), 8.26 (d, 1H), 8.13 (t, 1H), 7.91 (s, 1H), 7.68-7.56 (m, 2H), 7.28 (s, 1H), 6.66-6.60 (m, 2H), 6.06 (s, 1H), 2.76 (s, 3H), 1.51 (s, 3H); Chiral analytical SFC: RT=4.16 min, Column: Chiralpak IC-3, (4.6×150 mm, 3 m), 60% CO₂/Methanol, Flow=3.0 g/min.

7-Chloro-N-(1-(6,7-difluoro-1-oxo-1,2-dihydroisoquinolin-4-yl)ethyl)-N-methylindolizine-2-carboxamide—Enantiomer II (Compound 97), LCMS: m/z found 416.2/418.2 [M+H]⁺, RT=4.16 min (Method A); ¹H NMR (400 MHz, DMSO-d₆): δ 11.55 (bs, 1H), 8.26 (d, 1H), 8.13 (t, 1H), 7.91 (s, 1H), 7.68-7.56 (m, 2H), 7.28 (s, 1H), 6.66-6.60 (m, 2H), 6.06 (s, 1H), 2.76 (s, 3H), 1.51 (s, 3H); Chiral analytical SFC: RT=5.47 min, Column: Chiralpak IC-3, (4.6×150 mm, 3 μm), 60% CO₂/Methanol, Flow=3.0 g/min.

### N-(1-(6,7-Difluoro-1-oxo-1,2-dihydroisoquinolin-4-yl)ethyl)-6-fluoro-N-methylindolizine-2-carboxamide (Compounds 102 & 103)

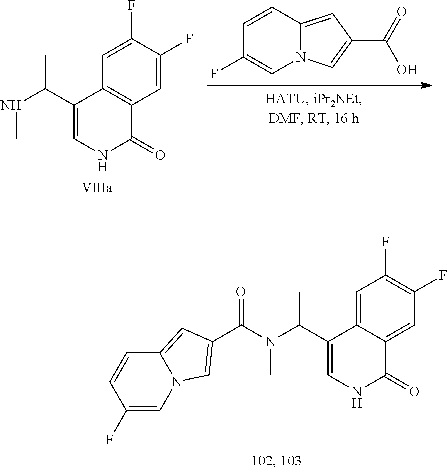

Racemic N-(1-(6,7-difluoro-1-oxo-1,2-dihydroisoquinolin-4-yl)ethyl)-6-fluoro-N-methylindolizine-2-carboxamide was synthesized in a similar manner as described above from 1H-6,7-difluoro-4-(1-(methylamino)ethyl)isoquinolin-1(2H)-one (VIIIa) and 6-fluoroindolizine-2-carboxylic acid. The enantiomers were subsequently separated by chiral SFC, Column: Chiralpak IC (250×30 mm, 5 μm), 60% CO₂/Methanol, Flow rate 110 g/min.

N-(1-(6,7-Difluoro-1-oxo-1,2-dihydroisoquinolin-4-yl)ethyl)-6-fluoro-N-methylindolizine-2-carboxamide—Enantiomer I (Compound 102), LCMS: m/z found 400.1 [M+H]⁺, RT=3.74 min (Method A); ¹H NMR (400 MHz, DMSO-d₆): δ 11.66 (bs, 1H), 8.42 (bs, 1H), 8.12 (t, 1H), 7.89 (s, 1H), 7.60-7.50 (m, 2H), 7.28 (s, 1H), 6.83 (t, 1H), 6.70 (s, 1H), 6.06 (s, 1H), 2.77 (s, 3H), 1.51 (s, 3H); Chiral analytical SFC: RT=3.10 min, Column: Chiralpak IC-3, (4.6×150 mm, 3 μm), 60% CO₂/Methanol, Flow=3.0 g/min.

N-(1-(6,7-Difluoro-1-oxo-1,2-dihydroisoquinolin-4-yl)ethyl)-6-fluoro-N-methylindolizine-2-carboxamide—Enantiomer II (Compound 103), LCMS: m/z found 400.1 [M+H]⁺, RT=3.74 min (Method A); ¹H NMR (400 MHz, DMSO-d₆): δ 11.66 (bs, 1H), 8.42 (bs, 1H), 8.12 (t, 1H), 7.89 (s, 1H), 7.60-7.50 (m, 2H), 7.28 (s, 1H), 6.83 (t, 1H), 6.70 (s, 1H), 6.06 (s, 1H), 2.77 (s, 3H), 1.51 (s, 3H); Chiral analytical SFC: RT=4.08 min, Column: Chiralpak IC-3, (4.6×150 mm, 3 μm), 60% CO₂/Methanol, Flow=3.0 g/min.

### N-(1-(6,7-Difluoro-1-oxo-1,2-dihydroisoquinolin-4-yl)ethyl)-7-fluoro-N-methylindolizine-2-carboxamide (Compounds 108 & 109)

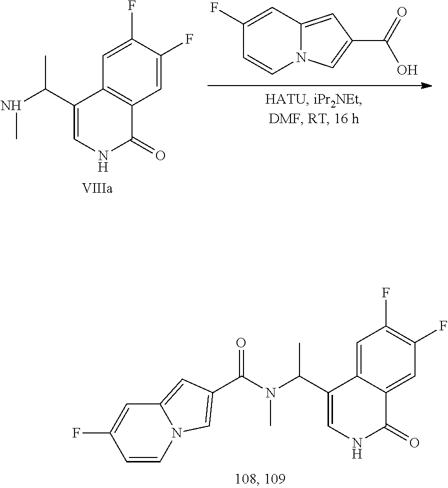

Racemic N-(1-(6,7-difluoro-1-oxo-1,2-dihydroisoquinolin-4-yl)ethyl)-7-fluoro-N-methylindolizine-2-carboxamide was synthesized in a similar manner as described above from 1H-6,7-difluoro-4-(1-(methylamino)ethyl)isoquinolin-1(2H)-one (VIIIa) and 7-fluoroindolizine-2-carboxylic acid. The enantiomers were subsequently separated by chiral SFC, Column: Chiralpak AS-H (250×30 mm, 5 μm), 60% CO₂/Methanol, Flow rate 105 g/min.

N-(1-(6,7-Difluoro-1-oxo-1,2-dihydroisoquinolin-4-yl)ethyl)-7-fluoro-N-methylindolizine-2-carboxamide—Enantiomer I (Compound 108), LCMS: m/z found 400.2 [M+H]⁺, RT=3.73 min (Method A); ¹H NMR (400 MHz, DMSO-d₆): δ 11.28 (bs, 1H), 8.29 (bs, 1H), 8.12 (t, 1H), 7.85 (s, 1H), 7.59 (s, 1H), 7.28-7.20 (m, 2H), 6.67 (t, 1H), 6.54 (s, 1H), 6.06 (s, 1H), 2.76 (s, 3H), 1.50 (s, 3H); Chiral analytical SFC: RT=1.44 min, Column: Chiralpak AS-3, (4.6×150 mm, 3 μm), 60% CO₂/(0.2% (7 M Methanolic Ammonia) in Acetonitrile:Methanol 1:1 v/v), Flow=3.0 g/min.

N-(1-(6,7-Difluoro-1-oxo-1,2-dihydroisoquinolin-4-yl)ethyl)-7-fluoro-N-methylindolizine-2-carboxamide—Enantiomer II (Compound 109), LCMS: m/z found 400.2 [M+H]⁺, RT=3.73 min (Method A); ¹H NMR (400 MHz, DMSO-d₆): δ 11.28 (bs, 1H), 8.29 (bs, 1H), 8.12 (t, 1H), 7.85 (s, 1H), 7.59 (s, 1H), 7.28-7.20 (m, 2H), 6.67 (t, 1H), 6.54 (s, 1H), 6.06 (s, 1H), 2.76 (s, 3H), 1.50 (s, 3H); Chiral analytical SFC: RT=2.77 min, Column: Chiralpak AS-3, (4.6×150 mm, 3 μm), 60% CO₂/(0.2% (7 M Methanolic Ammonia) in Acetonitrile:Methanol 1:1 v/v), Flow=3.0 g/min.

### 6-Chloro-N-(1-(6,7-difluoro-1-oxo-1,2-dihydroisoquinolin-4-yl)ethyl)-N-methylindolizine-2-carboxamide (Compounds 110 & 111)

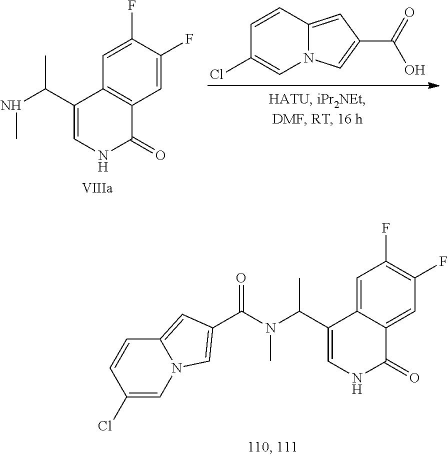 [SMILES: CNC(C)c1c[nH]c(=O)c2cc(F)c(F)cc12 ; O=C(O)c1cc2ccc(Cl)cn2c1 ; CC(c1c[nH]c(=O)c2cc(F)c(F)cc12)N(C)C(=O)c1cc2ccc(Cl)cn2c1]

Racemic 6-chloro-N-(1-(6,7-difluoro-1-oxo-1,2-dihydroisoquinolin-4-yl)ethyl)-N-methylindolizine-2-carboxamide was synthesized in a similar manner as described above from 1H-6,7-difluoro-4-(1-(methylamino)ethyl)isoquinolin-1(2H)-one (VIIIa) and 6-chloroindolizine-2-carboxylic acid. The enantiomers were subsequently separated by chiral SFC, Column: Chiralcel AS-H (250×30 mm, 5 μm), 60% CO₂/Methanol, Flow rate 100 g/min.

6-Chloro-N-(1-(6,7-difluoro-1-oxo-1,2-dihydroisoquinolin-4-yl)ethyl)-N-methylindolizine-2-carboxamide—Enantiomer I (Compound 110), LCMS: m/z found 416.2/418.1 [M+H]⁺, RT=4.20 min (Method A); ¹H NMR (400 MHz, DMSO-d₆): δ 11.7 (s, 1H), 8.5 (s, 1H), 8.14-8.09 (m, 1H), 7.80 (s, 1H), 7.50 (s, 1H), 7.49 (d, 1H), 7.20 (d, 1H), 6.77-6.70 (t, 2H), 6.06 (s, 1H), 2.76 (s, 3H), 1.52 (s, 3H); Chiral analytical SFC: RT=2.12 min, Column: Chiralcel AS-3, (4.6×150 mm, 3 μm), 60% CO₂/Methanol, Flow=3.0 g/min.

6-Chloro-N-(1-(6,7-difluoro-1-oxo-1,2-dihydroisoquinolin-4-yl)ethyl)-N-methylindolizine-2-carboxamide—Enantiomer II (Compound 111), LCMS: m/z found 416.2/418.1 [M+H]⁺, RT=4.20 min (Method A); ¹H NMR (400 MHz, DMSO-d₆): δ 11.7 (s, 1H), 8.5 (s, 1H), 8.14-8.09 (m, 1H), 7.80 (s, 1H), 7.50 (s, 1H), 7.49 (d, 1H), 7.20 (d, 1H), 6.77-6.70 (t, 2H), 6.06 (s, 1H), 2.76 (s, 3H), 1.52 (s, 3H); Chiral analytical SFC: RT=3.62 min, Column: Chiralcel AS-3, (4.6×150 mm, 3 μm), 60% CO₂/Methanol, Flow=3.0 g/min.

### N-(1-(6,7-Difluoro-1-oxo-1,2-dihydroisoquinolin-4-yl)ethyl)-N-(3-hydroxypropyl)-2-(1H-indol-2-yl)acetamide (Compounds 83 & 84)

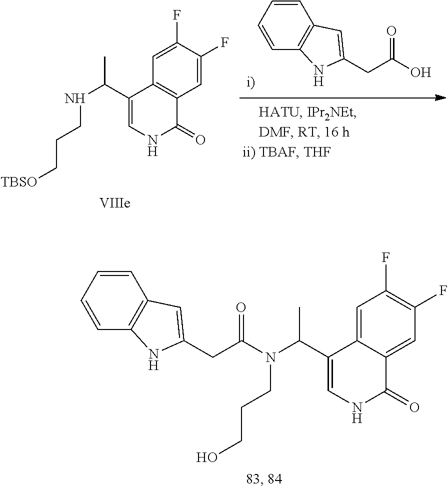

Racemic N-(1-(6,7-difluoro-1-oxo-1,2-dihydroisoquinolin-4-yl)ethyl)-N-(3-hydroxypropyl)-2-(1H-indol-2-yl)acetamide was synthesized in a similar manner as described above from 4-(1-((3-((tert-butyldimethylsilyl)oxy)propyl)amino)ethyl)-6,7-difluoroisoquinolin-1(2H)-one (VIIIe) and 2-(1H-indol-2-yl)acetic acid. The enantiomers were subsequently separated by chiral SFC, Column: Chiralcel OD-H (250×30 mm, 5 μm) 65% CO₂/MeOH, Flow rate 100 g/min.

N-(1-(6,7-Difluoro-1-oxo-1,2-dihydroisoquinolin-4-yl)ethyl)-N-(3-hydroxypropyl)-2-(1H-indol-2-yl)acetamide—Enantiomer I (Compound 83), LCMS: m/z found 440.2 [M+H]⁺, RT=3.55 min (Method A); ¹H NMR (400 MHz, DMSO-d₆): δ 11.79 (bs 1H), 11.01 (s, 1H), 8.12-8.07 (m, 1H), 7.51-7.47 (m, 1H), 7.39 (d, 1H), 7.33 (d, 1H), 7.28 (s, 1H), 7.01 (t, 1H) 6.93 (t, 1H), 6.18 (s, 1H), 5.98-5.89 (m, 1H), 4.45 (s, 1H), 3.87, (d, 2H), 3.32-3.12 (m, 4H), 1.52 (d, 3H), 1.43-1.35 (m, 1H), 0.98-0.94 (m, 1H); Chiral analytical SFC: RT=2.34 min, Column: Chiralcel OD-3, (4.6×150 mm, 3 μm), 60% CO₂/MeOH, Flow=3.0 g/min.

N-(1-(6,7-Difluoro-1-oxo-1,2-dihydroisoquinolin-4-yl)ethyl)-N-(3-hydroxypropyl)-2-(1H-indol-2-yl)acetamide—Enantiomer II (Compound 84), LCMS: m/z found 440.2 [M+H]⁺, RT=3.55 min (Method A); ¹H NMR (400 MHz, DMSO-d₆): δ 11.79 (bs 1H), 11.01 (s, 1H), 8.12-8.07 (m, 1H), 7.51-7.47 (m, 1H), 7.39 (d, 1H), 7.33 (d, 1H), 7.28 (s, 1H), 7.01 (t, 1H) 6.93 (t, 1H), 6.18 (s, 1H), 5.98-5.89 (m, 1H), 4.45 (s, 1H), 3.87, (d, 2H), 3.32-3.12 (m, 4H), 1.52 (d, 3H), 1.43-1.35 (m, 1H), 0.98-0.94 (m, 1H); Chiral analytical SFC: RT=3.47 min, Column: Chiralcel OD-3, (4.6×150 mm, 3 μm), 60% CO₂/MeOH, Flow=3.0 g/min.

### N-(1-(6,7-Difluoro-1-oxo-1,2-dihydroisoquinolin-4-yl)ethyl)-5,5-difluoro-N-methyl-4,5,6,7-tetrahydro-1H-indole-2-carboxamide (Compounds 88 & 89)

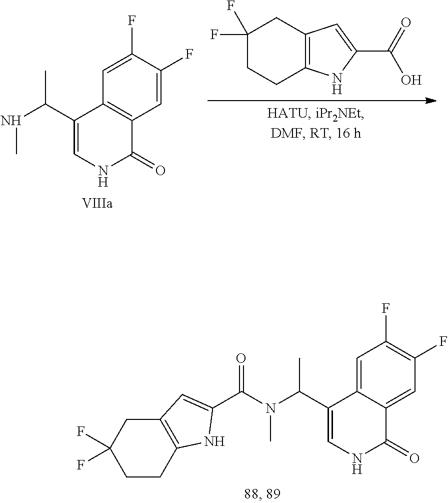

Racemic N-(1-(6,7-difluoro-1-oxo-1,2-dihydroisoquinolin-4-yl)ethyl)-5,5-difluoro-N-methyl-4,5,6,7-tetrahydro-1H-indole-2-carboxamide was synthesized in a similar manner as described above from 1H-6,7-difluoro-4-(1-(methylamino)ethyl)isoquinolin-1(2H)-one (VIIIa) and 5,5-difluoro-4,5,6,7-tetrahydro-1H-indole-2-carboxylic acid. The enantiomers were subsequently separated by chiral SFC, Column: Chiralcel-OX-H (250×30 mm, 5 m), 60% CO₂/Methanol, Flow rate 100 g/min.

N-(1-(6,7-Difluoro-1-oxo-1,2-dihydroisoquinolin-4-yl)ethyl)-5,5-difluoro-N-methyl-4,5,6,7-tetrahydro-1H-indole-2-carboxamide—Enantiomer I (Compound 88), LCMS: m/z found 422.2 [M+H]⁺, RT=3.86 min (Method A); ¹H NMR (400 MHz, DMSO-d₆): δ 11.37 (s, 2H), 8.12-8.08 (m, 1H), 7.61-7.59 (m, 1H), 7.27 (s, 1H), 6.34 (s, 1H), 6.06-6.04 (m, 1H), 2.96 (t, 2H), 2.80-2.74 (m, 5H), 2.26-2.15 (m, 2H), 1.45 (d, 3H); Chiral analytical SFC: RT=1.56 min, Column: Chiralcel-OX-3, (4.6×150 mm, 3 μm), 60% CO₂/Methanol, Flow=3.0 g/min.

N-(1-(6,7-Difluoro-1-oxo-1,2-dihydroisoquinolin-4-yl)ethyl)-5,5-difluoro-N-methyl-4,5,6,7-tetrahydro-1H-indole-2-carboxamide—Enantiomer II (Compound 89), LCMS: m/z found 422.2 [M+H]⁺, RT=3.86 min (Method A); ¹H NMR (400 MHz, DMSO-d₆): δ 11.37 (s, 2H), 8.12-8.08 (m, 1H), 7.61-7.59 (m, 1H), 7.27 (s, 1H), 6.34 (s, 1H), 6.06-6.04 (m, 1H), 2.96 (t, 2H), 2.80-2.74 (m, 5H), 2.26-2.15 (m, 2H), 1.45 (d, 3H); Chiral analytical SFC: RT=1.96 min, Column: Chiralcel-OX-3, (4.6×150 mm, 3 μm), 60% CO₂/Methanol, Flow=3.0 g/min.

### N-(1-(6,7-Difluoro-1-oxo-1,2-dihydroisoquinolin-4-yl)ethyl)-N-methyl-1H-indole-3-carboxamide (Compounds 53 & 54)

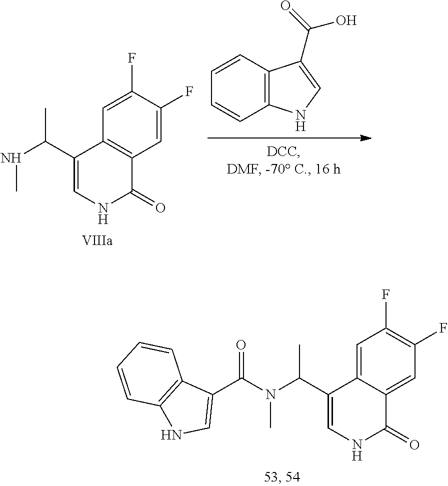

To a stirred solution of 0.2 g (1.24 mmol, 1.0 eq.) of 1H-indole-3-carboxylic acid in 3 mL of DMF under a nitrogen atmosphere at room temperature was added 0.28 g (1.36 mmol, 1.1 eq.) of dicyclohexyl carbodiimide. The mixture was stirred for 15 min and 0.28 g (1.24 mmol, 1.0 eq.) of 6,7-difluoro-4-(1-(methylamino)ethyl)isoquinolin-1(2H)-one (VIIIa) was added. The reaction mixture was then heated at 70° C. for 16 h. The mixture was allowed to cool to room temperature and diluted with water (30 mL). The resulting precipitate was collected by filtration, washed with pentane (10 mL) and dried under high vacuum. The residue was purified by preparative HPLC (Column: XSELECT PHENYL-HEXYL (150×19 mm, 5 μm), Mobile phase A: 10 mM Ammonium Bicarbonate in water, Mobile phase B: 100% Acetonitrile; Method T/% B=0/60, 1/60, 11/60, 11.1/100, 13/100, 13.1/60, 15/60; Flow rate: 19 mL/min) to provide 70 mg (0.18 mmol, 15% yield) of racemic N-(1-(6,7-difluoro-1-oxo-1,2-dihydroisoquinolin-4-yl)ethyl)-N-methyl-1H-indole-3-carboxamide. The enantiomers were subsequently separated by chiral SFC, Column: Chiralpak IC (250×30 mm, 5 μm), 65% CO₂/(30 mM ammonia in methanol), Flow rate=90 g/min.

N-(1-(6,7-Difluoro-1-oxo-1,2-dihydroisoquinolin-4-yl)ethyl)-N-methyl-1H-indole-3-carboxamide—Enantiomer I (Compound 53), LCMS: m/z found 382.2 [M+H]⁺, RT=3.19 min (Method A); ¹H NMR (400 MHz, DMSO-d₆): δ 11.59 (bs, 2H), 8.14-8.09 (m, 1H), 7.77-7.71 (m, 3H), 7.44-7.41 (m, 1H), 7.29 (s, 1H), 7.15-7.06 (m, 2H), 6.13-6.06 (m, 1H), 2.76 (s, 3H), 1.54 (d, 3H); Chiral analytical SFC: RT=3.75 min, Column: Chiralpak IC-3, (4.6×150 mm, 3 μm), 65% CO₂/Methanol, Flow=3.0 g/min.

N-(1-(6,7-Difluoro-1-oxo-1,2-dihydroisoquinolin-4-yl)ethyl)-N-methyl-1H-indole-3-carboxamide—Enantiomer II (Compound 54), LCMS: m/z found 382.2 [M+H]⁺, RT=3.19 min (Method A); ¹H NMR (400 MHz, DMSO-d₆): δ 11.59 (bs, 2H), 8.14-8.09 (m, 1H), 7.77-7.71 (m, 3H), 7.44-7.41 (m, 1H), 7.29 (s, 1H), 7.15-7.06 (m, 2H), 6.13-6.06 (m, 1H), 2.76 (s, 3H), 1.54 (d, 3H); Chiral analytical SFC: RT=4.64 min, Column: Chiralpak IC-3, (4.6×150 mm, 3 μm), 65% CO₂/Methanol, Flow=3.0 g/min.

### N-(1-(6,7-Difluoro-1-oxo-1,2-dihydroisoquinolin-4-yl)ethyl)-N-methyl-2H-indazole-3-carboxamide (Compounds 55 & 56)

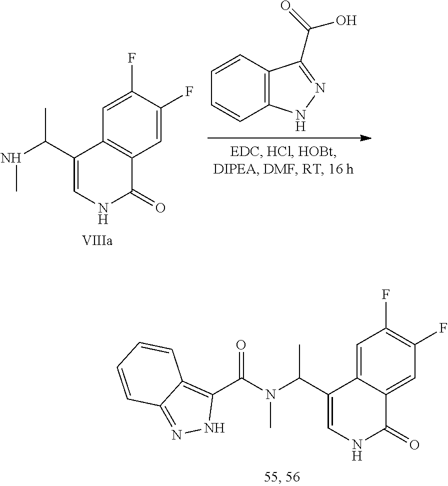

To a stirred solution of 136 mg (0.84 mmol, 1.0 eq.) of 1H-indazole-3-carboxylic acid in 3 mL of DMF at 0° C. was added 0.13 mL (1.01 mmol, 1.25 eq.) of N,N-diisopropylethylamine followed by 176 mg (0.92 mmol, 1.1 eq.) of N-Ethyl-N′-(3-dimethylaminopropyl)carbodiimide hydrochloride and 113 mg (0.84 mmol, 1.0 eq.) of HOBt monohydrate. The mixture was stirred for 15 min and 0.2 g (0.84 mmol, 1.0 eq.) of 6,7-difluoro-4-(1-(methylamino)ethyl)isoquinolin-1(2H)-one (VIIIa) was added and the reaction mixture was stirred at room temperature for 16 h. The mixture was diluted with water (30 mL) and stirred for 15 min. The resulting precipitate was collected by filtration, washed with n-pentane (10 mL), dried under high vacuum and then purified by preparative HPLC (Column: XSELECT KROMOSIL (150×25 mm, 10 μm), Mobile phase A: 10 mM Ammonium Bicarbonate in water, Mobile phase B: 100% Acetonitrile; Method T/% B=0/30, 1/30, 11/50, 11.1/100, 13/100, 13.1/30, 15/30; Flow rate: 19 mL/min) to provide 110 mg (0.28 mmol, 34% yield) of N-(1-(6,7-difluoro-1-oxo-1,2-dihydroisoquinolin-4-yl)ethyl)-N-methyl-2H-indazole-3-carboxamide. The enantiomers were subsequently separated by chiral SFC, Column: Chiralpak AD-H (250×30 mm, 5 μm), 75% CO₂/(30 mM methanolic ammonia in isopropanol), Flow rate 70 g/min.

N-(1-(6,7-Difluoro-1-oxo-1,2-dihydroisoquinolin-4-yl)ethyl)-N-methyl-2H-indazole-3-carboxamide—Enantiomer I (Compound 55), LCMS: m/z found 383.2 [M+H]⁺, RT=3.26 min (Method A); ¹H NMR (400 MHz, DMSO-d₆, 90° C.): δ 13.25 (bs, 1H), 11.36 (bs, 1H), 8.12-8.07 (m, 1H), 7.99 (d, 1H), 7.74-7.69 (m, 1H), 7.59 (d, 1H), 7.43-7.39 (m, 1H), 7.25-7.21 (m, 2H), 6.26-6.21 (m, 1H), 2.85 (s, 3H), 1.57 (d, 3H); Chiral analytical SFC: RT=5.60 min, Column: Chiralpak AD-3, (4.6×150 mm, 3 μm), 80% CO₂/(0.5% Isopropylamine in Isopropanol), Flow=3.0 g/min.

N-(1-(6,7-Difluoro-1-oxo-1,2-dihydroisoquinolin-4-yl)ethyl)-N-methyl-2H-indazole-3-carboxamide—Enantiomer II (Compound 56), LCMS: m/z found 383.2 [M+H]⁺, RT=3.26 min (Method A); ¹H NMR (400 MHz, DMSO-d₆, 90° C.): δ 13.25 (bs, 1H), 11.36 (bs, 1H), 8.12-8.07 (m, 1H), 7.99 (d, 1H), 7.74-7.69 (m, 1H), 7.59 (d, 1H), 7.43-7.39 (m, 1H), 7.25-7.21 (m, 2H), 6.26-6.21 (m, 1H), 2.85 (s, 3H), 1.57 (d, 3H); Chiral analytical SFC: RT=7.44 min, Column: Chiralpak AD-3, (4.6×150 mm, 3 μm), 80% CO₂/(0.5% Isopropylamine in Isopropanol), Flow=3.0 g/min.

### N-(1-(6,7-Difluoro-1-oxo-1,2-dihydroisoquinolin-4-yl)ethyl)-2-(1H-indol-2-yl)-N-methylacetamide (Compound 59)

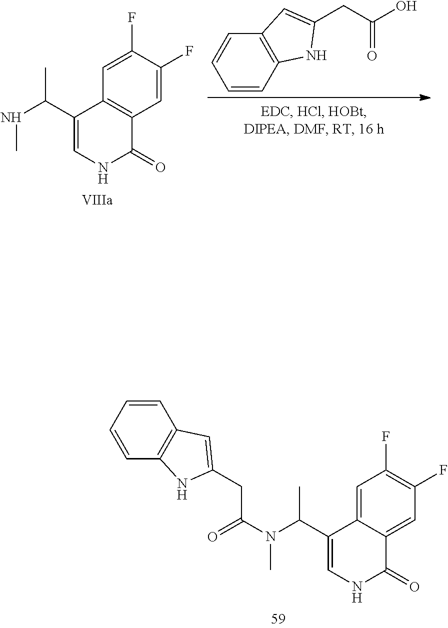

Racemic N-(1-(6,7-difluoro-1-oxo-1,2-dihydroisoquinolin-4-yl)ethyl)-2-(1H-indol-2-yl)-N-methylacetamide was synthesized in a similar manner as described above from 1H-6,7-difluoro-4-(1-(methylamino)ethyl)isoquinolin-1(2H)-one (VIIIa) and 2-(1H-indol-2-yl)acetic acid. LCMS: m/z found 396.3 [M+H]⁺, RT=3.72 min (Method A); ¹H NMR (400 MHz, DMSO-d₆): δ 11.61 (bs, 1H), 10.97 (bs, 1H), 8.11-8.06 (m, 1H), 7.57-7.52 (m, 1H), 7.37-7.30 (m, 2H), 7.21 (s, 1H), 7.01-6.90 (m, 2H), 6.13 (s, 1H), 5.98-5.93 (m, 1H), 3.90-3.81 (m, 2H), 2.64 (s, 3H), 1.40 (d, 3H).

### N-(1-(6,7-Difluoro-1-oxo-1,2-dihydroisoquinolin-4-yl)ethyl)-N,3,3-trimethylindoline-2-carboxamide (Compounds 112, 113, 114, & 115)

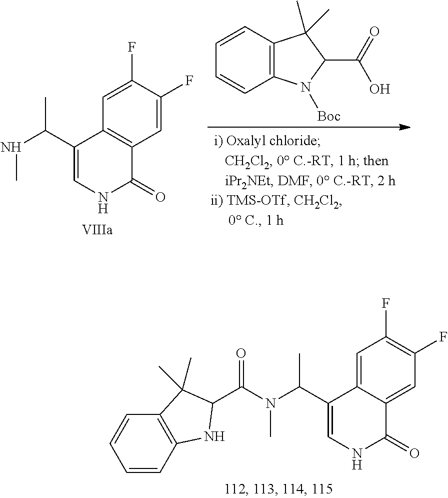

The enantiomers of known intermediate 1-(tert-butoxycarbonyl)-3,3-dimethylindoline-2-carboxylic acid were separated by chiral SFC, Column: Chiralpak IC (250×30 mm, 5 μm), 80% CO₂/(Acetonitrile:IPA 1:1 v/v), Flow rate 100 g/min and carried each individually in the next steps. To a stirred solution of 100 mg (0.34 mmol, 1.0 eq.) of 1-(tert-butoxycarbonyl)-3,3-dimethylindoline-2-carboxylic acid (first eluting enantiomer) in 2 mL of methylene chloride at 0° C. was added 65 μL (0.69 mmol, 2.0 eq.) of oxalyl chloride and the mixture was stirred at room temperature for 1 h. The reaction mixture was concentrated under reduced pressure, and azeotropically dried with toluene (2×5 mL). The residue was diluted with dry methylene chloride (2.0 mL) and added into a stirred solution of 90 mg (0.34 mmol, 1.0 eq.) of 7,8-difluoro-4-(1-(methylamino)ethyl)isoquinolin-1(2H)-one (VIIIa) in 1 mL of DMF at 0° C. and stirring was continued at room temperature for 2 h. The volatiles were removed under reduce pressure and the reaction mixture was poured into ice-cold water (20 mL). The precipitated solid was filtered and washed with water (20 ml). The residue was triturated with n-pentane (10 mL) and filtered to provide 140 mg (0.27 mmol, 79% yield) of tert-butyl 2-((1-(7,8-difluoro-1-oxo-1,2-dihydroisoquinolin-4-yl)ethyl)(methyl)carbamoyl)-3,3-dimethylindoline-1-carboxylate as an off-white solid mixture of two diastereoisomers.

tert-Butyl 2-((1-(7,8-difluoro-1-oxo-1,2-dihydroisoquinolin-4-yl)ethyl)(methyl)carbamoyl)-3,3-dimethylindoline-1-carboxylate (mixture of two diastereoisomers) was converted to N-(1-(6,7-difluoro-1-oxo-1,2-dihydroisoquinolin-4-yl)ethyl)-N-3,3-trimethylindoline-2-carboxamide by treatment with TMSOTf in a similar manner as described above. The two diastereoisomers were subsequently separated by chiral SFC, Column: DCPAK P4CP (250×21 mm, 5 μm) 70% CO₂/Methanol, Flow rate 70 g/min.

The other two stereoisomers of N-(1-(6,7-difluoro-1-oxo-1,2-dihydroisoquinolin-4-yl)ethyl)-N,3,3-trimethylindoline-2-carboxamide were synthesized in a similar manner as described above from 7,8-difluoro-4-(1-(methylamino)ethyl)isoquinolin-1(2H)-one (VIIIa) and 1-(tert-butoxycarbonyl)-3,3-dimethylindoline-2-carboxylic acid (second eluting enantiomer), and were subsequently separated by chiral SFC under the same conditions as above.

N-(1-(6,7-Difluoro-1-oxo-1,2-dihydroisoquinolin-4-yl)ethyl)-N,3,3-trimethylindoline-2-carboxamide—Stereoisomer IIA (Compound 112), LCMS: m/z found 412.2 [M+H]⁺, RT=3.50 min (Method A); ¹H NMR (400 MHz, DMSO-d₆): δ 11.66 (s, 1H), 8.13-8.08 (m, 1H), 7.58-53 (m, 1H), 7.28 (s, 1H), 6.95-6.89 (m, 2H), 6.57-6.52 (m, 2H), 6.01-5.95 (m, 1H), 5.71 (s, 1H), 4.38 (s, 1H), 2.71 (s, 3H), 1.42 (d, 3H), 1.24 (s, 3H), 1.01 (s, 3H); Chiral analytical SFC: RT=3.28 min, Column: Chiralpak AS-3, (4.6×150 mm, 3 μm), 70% CO₂/(0.5% DEA in Methanol), Flow=3.0 g/min.

N-(1-(6,7-Difluoro-1-oxo-1,2-dihydroisoquinolin-4-yl)ethyl)-N,3,3-trimethylindoline-2-carboxamide—Stereoisomer IA (Compound 113, enantiomer of Compound 112), LCMS: m/z found 412.2 [M+H]⁺, RT=3.50 min (Method A); ¹H NMR (400 MHz, DMSO-d₆): δ 11.66 (s, 1H), 8.13-8.08 (m, 1H), 7.58-53 (m, 1H), 7.28 (s, 1H), 6.95-6.89 (m, 2H), 6.57-6.52 (m, 2H), 6.01-5.95 (m, 1H), 5.71 (s, 1H), 4.38 (s, 1H), 2.71 (s, 3H), 1.42 (d, 3H), 1.24 (s, 3H), 1.01 (s, 3H); Chiral analytical SFC: RT=1.87 min, Column: Chiralpak AS-3, (4.6×150 mm, 3 m), 70% CO₂/(0.5% DEA in Methanol), Flow=3.0 g/min.

N-(1-(6,7-Difluoro-1-oxo-1,2-dihydroisoquinolin-4-yl)ethyl)-N,3,3-trimethylindoline-2-carboxamide—Stereoisomer IB (Compound 114), LCMS: m/z found 412.2 [M+H]⁺, RT=3.33 min (Method A); ¹H NMR (400 MHz, DMSO-d₆): δ 11.28 (s, 1H), 8.14-8.09 (m, 1H), 7.59-7.53 (m, 1H), 7.24 (m, 1H), 6.95-6.91 (m, 2H), 6.59-6.53 (m, 2H), 5.97-5.91 (m, 1H), 5.63 (s, 1H), 4.39 (s, 1H), 2.69 (s, 3H), 1.38 (d, 3H), 1.35 (s, 3H), 1.21 (s, 3H); Chiral analytical SFC: RT=2.00 min, Column: Chiralpak IA-3, (4.6×150 mm, 3 μm), 60% CO₂/Methanol, Flow=3.0 g/min.
N-(1-(6,7-Difluoro-1-oxo-1,2-dihydroisoquinolin-4-yl)ethyl)-N,3,3-trimethylindoline-2-carboxamide—Stereoisomer IIB (Compound 115, enantiomer of Compound 114), LCMS: m/z found 412.2 [M+H]⁺, RT=3.33 min (Method A); ¹H NMR (400 MHz, DMSO-d₆): δ 11.28 (s, 1H), 8.14-8.09 (m, 1H), 7.59-7.53 (m, 1H), 7.24 (m, 1H), 6.95-6.91 (m, 2H), 6.59-6.53 (m, 2H), 5.97-5.91 (m, 1H), 5.63 (s, 1H), 4.39 (s, 1H), 2.69 (s, 3H), 1.38 (d, 3H), 1.35 (s, 3H), 1.21 (s, 3H); Chiral analytical SFC: RT=2.95 min, Column: Chiralpak IA-3, (4.6×150 mm, 3 μm), 60% CO₂/Methanol, Flow=3.0 g/min.

### N-(1-(6,7-Difluoro-1-oxo-1,2-dihydroisoquinolin-4-yl)ethyl)-3-fluoro-N-isobutyl-4-(trifluoromethyl)benzamide (Compound 117)

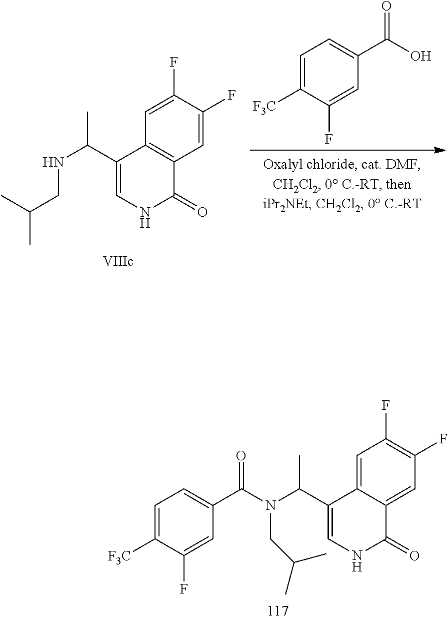

### Synthesis of 3-fluoro-4-(trifluoromethyl)benzoyl chloride

To a stirred solution of 50 mg (0.24 mmol, 1.0 eq.) of 3-fluoro-4-(trifluoromethyl)benzoic acid in 5 mL of methylene chloride at 0° C. under a nitrogen atmosphere were added 0.04 mL (0.48 mmol, 2.0 eq.) of oxalyl chloride and catalytic DMF, and the reaction mixture was allowed to stir at room temperature for 1 h. The mixture was concentrated under vacuum and azeotropically dried twice with 5 mL of toluene to obtain 55 mg of 3-fluoro-4-(trifluoromethyl)benzoyl chloride.

### Synthesis of N-(1-(6,7-difluoro-1-oxo-1,2-dihydroisoquinolin-4-yl)ethyl)-3-fluoro-N-isobutyl-4-(trifluoromethyl)benzamide (Compound 117)

To a stirred solution of 30 mg (0.11 mmol, 1.0 eq.) of 6,7-difluoro-4-(1-(isobutylamino)ethyl)isoquinolin-1(2H)-one (VIIIc) and 41.5 mg (0.32 mmol, 3.0 eq.) of N,N-diisopropylethylamine in 3 mL of methylene chloride at 0° C. was added a solution of 48.5 mg (0.21 mmol, 2.0 eq.) of 3-fluoro-4-(trifluoromethyl)benzoyl chloride in 1 mL of methylene chloride and the mixture was stirred at room temperature for 1 h. The solvent was removed in vacuo and the residue was diluted with water (5 mL) and stirred for 10 min. The resulting suspension was filtered, and 40 mg of crude solid was collected. The procedure was repeated at the same scale and the product was purified by reverse phase preparative HPLC (Column: X select C8 (19×250 mm, 5 μm), Mobile phase A: 10 mM ammonium bicarbonate in water, Mobile phase B: Acetonitrile; Gradient (Time/% B): 0/40,10/60,14/60,15/95,17/95,18/40,22/40; Flow: 17 mL/min) to provide 8 mg (0.017 mmol, 7.3% yield) of racemic N-(1-(6,7-difluoro-1-oxo-1,2-dihydroisoquinolin-4-yl)ethyl)-3-fluoro-N-isobutyl-4-(trifluoromethyl)benzamide. LCMS: m/z found 471.2 [M+H]⁺, RT=7.20 min (Method A); ¹H NMR (400 MHz, DMSO-d₆): δ 11.66 (br s, 1H), 8.13 (t, 1H), 7.86 (t, 1H), 7.59-7.56 (m, 2H), 7.45 (s, 1H), 7.33-7.31 (d, 1H), 6.04 (m, 1H), 2.89-2.81 (m, 2H), 1.62 (d, 3H), 1.23 (m, 1H), 0.38 (m, 6H).

### General Procedures for the Syntheses of Compounds 119-144

General Procedure II

To a stirred solution of the carboxylic acid (1.0 eq.) in DMF (10 volumes) at room temperature were added N,N-diisopropylethyl amine (3.0 eq.) and HATU (2.0 eq.) and the reaction mixture was stirred at room temperature for 30 minutes. 6,7-Difluoro-4-(1-(methylamino)ethyl)isoquinolin-1(2H)-one (VIIIa, 1.0 eq.) was added and the resulting reaction mixture was stirred at room temperature for 2 h. The reaction mixture was poured onto ice-water and the precipitated solid was collected by filtration, washed with water and triturated with diethyl ether and n-pentane to provide the corresponding amide.

General Procedure III

To a stirred solution of the carboxylic acid (1.0 eq.) in THF (10 volumes) at room temperature were added N,N-diisopropylethyl amine (3.0 eq.), 1-ethyl-3-(3-dimethylaminopropyl)carbodiimide hydrochloride (1.5 eq.) and hydroxybenzotriazole monohydrate (1.5 eq.) and the reaction mixture was stirred at room temperature for 15 minutes. 6,7-Difluoro-4-(1-(methylamino)ethyl)isoquinolin-1(2H)-one (VIIIa, 1.0 eq.) was added and the resulting reaction mixture was stirred at room temperature for 2 to 16 h. After completion of the reaction, the mixture was poured onto ice-water and the precipitated solid was collected by filtration, washed with water and triturated with diethyl ether and n-pentane to provide the corresponding amide.

General Procedure IV

To a stirred solution of 6,7-difluoro-4-(1-(methylamino)ethyl)isoquinolin-1(2H)-one (VIIIa, 1.0 eq.) in THF (20 volumes) was added of triethylamine (1.5 eq.), followed by the acyl chloride at 0° C. and the reaction mixture was stirred at 0° C. to room temperature for 1 h. After completion of reaction, the organic volatiles were removed under reduced pressure and the obtained residue was stirred with a saturated NaHCO₃ solution. The precipitate was collected by filtration and dried under vacuum and triturated with acetone to provide the corresponding amide.

### N-(1-(6,7-Difluoro-1-oxo-1,2-dihydroisoquinolin-4-yl)ethyl)-3-fluoro-N-methyl-4-(trifluoromethyl) benzamide (Compounds 119 & 120)

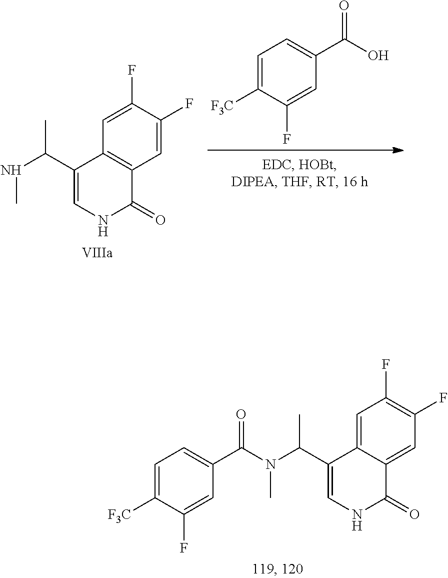

Racemic N-(1-(6,7-difluoro-1-oxo-1,2-dihydroisoquinolin-4-yl)ethyl)-3-fluoro-N-methyl-4-(trifluoromethyl) benzamide was prepared according to General procedure III from 3-fluoro-4-(trifluoromethyl)benzoic acid. The enantiomers were subsequently separated by chiral SFC, Column: Chiralpak IC (250×30 mm, 5 μm), 75% CO₂/Methanol, Flow rate 100 g/min.

N-(1-(6,7-Difluoro-1-oxo-1,2-dihydroisoquinolin-4-yl)ethyl)-3-fluoro-N-methyl-4-(trifluoromethyl) benzamide—Enantiomer I (Compound 119), LCMS: m/z found 429.3 [M+H]⁺, RT=1.95 min (Method C); ¹H NMR (400 MHz, DMSO-d₆): δ 11.69 (s, 1H), 8.15 (t, 1H), 7.85 (d, 1H), 7.49-7.44 (m, 1H), 7.64-7.61 (m, 1H), 7.56 (d, 1H), 7.32-7.30 (m, 1H), 6.03 (q, 1H), 2.45 (s, 3H), 1.54 (d, 3H); Chiral analytical SFC: RT=2.88 min, Column: Chiralpak IC-3, (4.6×150 mm, 3 m), 80% CO₂/Methanol, Flow=3.0 g/min.

N-(1-(6,7-Difluoro-1-oxo-1,2-dihydroisoquinolin-4-yl)ethyl)-3-fluoro-N-methyl-4-(trifluoromethyl) benzamide—Enantiomer II (Compound 120), LCMS: m/z found 429.3 [M+H]⁺, RT=1.95 min (Method C); ¹H NMR (400 MHz, DMSO-d₆): δ 11.69 (s, 1H), 8.15 (t, 1H), 7.85 (d, 1H), 7.49-7.44 (m, 1H), 7.64-7.61 (m, 1H), 7.56 (d, 1H), 7.32-7.30 (m, 1H), 6.03 (q, 1H), 2.45 (s, 3H), 1.54 (d, 3H); Chiral analytical SFC: RT=3.27 min, Column: Chiralpak IC-3, (4.6×150 mm, 3 μm), 80% CO₂/Methanol, Flow=3.0 g/min.

### 4-Chloro-N-(1-(6,7-difluoro-1-oxo-1,2-dihydroisoquinolin-4-yl)ethyl)-3-fluoro-N-methylbenzamide (Compounds 121 & 122)

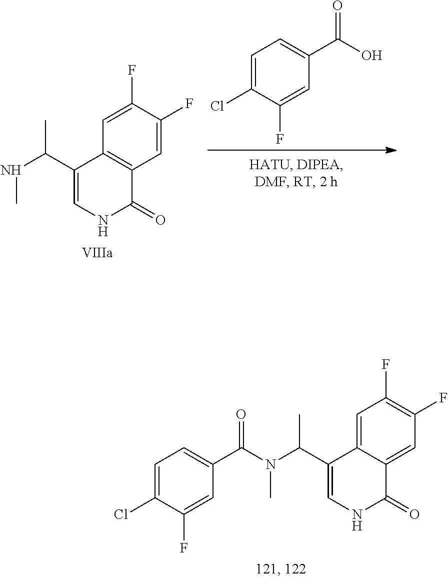

Racemic 4-chloro-N-(1-(6,7-difluoro-1-oxo-1,2-dihydroisoquinolin-4-yl)ethyl)-3-fluoro-N-methylbenzamide was prepared according to General procedure II from 4-chloro-3-fluorobenzoic acid. The enantiomers were subsequently separated by chiral SFC, Column: Chiralpak IC (250×30 mm, 5 μm), 70% CO₂/Methanol, Flow rate 100 g/min.

4-Chloro-N-(1-(6,7-difluoro-1-oxo-1,2-dihydroisoquinolin-4-yl)ethyl)-3-fluoro-N-methylbenzamide—Enantiomer I (Compound 121), LCMS: m/z found 395.2 [M+H]⁺, RT=1.64 min (Method D); ¹H NMR (400 MHz, DMSO-d₆): δ 11.67 (s, 1H), 8.14 (t, 1H), 7.67-7.57 (m, 2H), 7.46-7.44 (d, 1H), 7.28 (s, 1H), 7.16 (d, 1H), 6.00 (q, 1H), 2.49 (s, 3H), 1.53 (d, 3H); Chiral analytical SFC: RT=3.82 min, Column: Chiralpak IC-3, (4.6×150 mm, 3 μm), 70% CO₂/Methanol, Flow=3.0 g/min.

4-Chloro-N-(1-(6,7-difluoro-1-oxo-1,2-dihydroisoquinolin-4-yl)ethyl)-3-fluoro-N-methylbenzamide—Enantiomer II (Compound 122), LCMS: m/z found 395.2 [M+H]⁺, RT=1.64 min (Method D); ¹H NMR (400 MHz, DMSO-d₆): δ 11.67 (s, 1H), 8.14 (t, 1H), 7.67-7.57 (m, 2H), 7.46-7.44 (d, 1H), 7.28 (s, 1H), 7.16 (d, 1H), 6.00 (q, 1H), 2.49 (s, 3H), 1.53 (d, 3H); Chiral analytical SFC: RT=4.44 min, Column: Chiralpak IC-3, (4.6×150 mm, 3 μm), 70% CO₂/Methanol, Flow=3.0 g/min.

### N-(1-(6,7-Difluoro-1-oxo-1,2-dihydroisoquinolin-4-yl)ethyl)-3,4,5-trifluoro-N-methylbenzamide (Compounds 123 & 124)

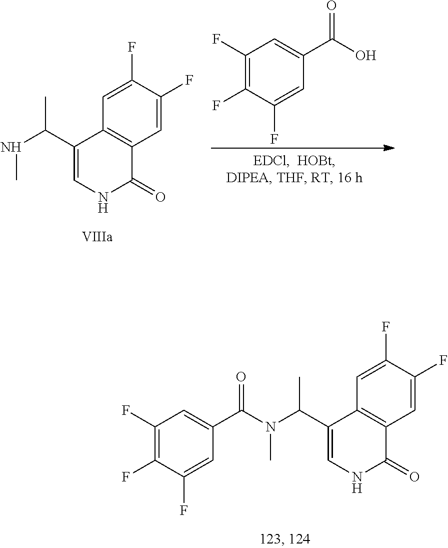

Racemic N-(1-(6,7-difluoro-1-oxo-1,2-dihydroisoquinolin-4-yl)ethyl)-3,4,5-trifluoro-N-methylbenzamide was prepared according to General procedure III from 3,4,5-trifluorobenzoic acid. The enantiomers were subsequently separated by chiral SFC, Column: Chiralpak IC (250×30 mm, 5 μm), 75% CO₂/Methanol, Flow rate 100 g/min.

N-(1-(6,7-Difluoro-1-oxo-1,2-dihydroisoquinolin-4-yl)ethyl)-3,4,5-trifluoro-N-methylbenzamide—Enantiomer I (Compound 123), LCMS: m/z found 397.3 [M+H]⁺, RT=1.85 min (Method C); ¹H NMR (400 MHz, DMSO-d₆): δ 11.67 (s, 1H), 8.13 (t, 1H), 7.60-7.55 (m, 1H), 7.36 (m, 2H), 7.29 (s, 1H), 5.99 (q, 1H), 2.49 (s, 3H), 1.52 (d, 3H); Chiral analytical SFC: RT=3.53 min, Column: Chiralpak IC-3, (4.6×150 mm, 3 μm), 80% CO₂/Methanol, Flow=3.0 g/min.

N-(1-(6,7-Difluoro-1-oxo-1,2-dihydroisoquinolin-4-yl)ethyl)-3,4,5-trifluoro-N-methylbenzamide—Enantiomer II (Compound 124), LCMS: m/z found 397.3 [M+H]⁺, RT=1.85 min (Method C); ¹H NMR (400 MHz, DMSO-d₆): δ 11.67 (s, 1H), 8.13 (t, 1H), 7.60-7.55 (m, 1H), 7.36 (m, 2H), 7.29 (s, 1H), 5.99 (q, 1H), 2.49 (s, 3H), 1.52 (d, 3H); Chiral analytical SFC: RT=4.17 min, Column: Chiralpak IC-3, (4.6×150 mm, 3 m), 80% CO₂/Methanol, Flow=3.0 g/min.

### N-(1-(6,7-Difluoro-1-oxo-1,2-dihydroisoquinolin-4-yl)ethyl)-3-(difluoromethyl)-N-methylbenzamide (Compounds 125 & 126)

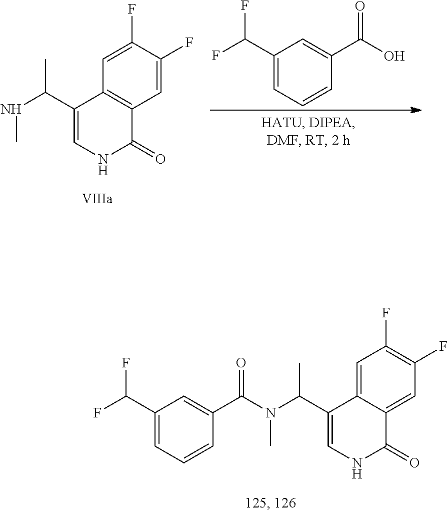

Racemic N-(1-(6,7-difluoro-1-oxo-1,2-dihydroisoquinolin-4-yl)ethyl)-3-(difluoromethyl)-N-methylbenzamide was prepared according to General procedure II from 3-(difluoromethyl)benzoic acid. The enantiomers were subsequently separated by chiral SFC, Column: Chiralpak IC (250×30 mm, 5 μm), 75% CO₂/Methanol, Flow rate 100 g/min.

N-(1-(6,7-Difluoro-1-oxo-1,2-dihydroisoquinolin-4-yl)ethyl)-3-(difluoromethyl)-N-methylbenzamide—Enantiomer I (Compound 125), LCMS: m/z found 393.2 [M+H]⁺, RT=1.52 min (Method D); ¹H NMR (400 MHz, DMSO-d₆): δ 11.67 (s, 1H), 8.15 (t, 1H), 7.66-7.46 (m, 5H), 7.28 (s, 1H), 7.06 (t, 1H), 6.05 (q, 1H), 2.47 (s, 3H), 1.55 (d, 3H); Chiral analytical SFC: RT=5.42 min, Column: Chiralpak IC-3, (4.6×150 mm, 3 μm), 80% CO₂/Methanol, Flow=3.0 g/min.

N-(1-(6,7-Difluoro-1-oxo-1,2-dihydroisoquinolin-4-yl)ethyl)-3-(difluoromethyl)-N-methylbenzamide—Enantiomer II (Compound 126), LCMS: m/z found 393.2 [M+H]⁺, RT=1.52 min (Method D); ¹H NMR (400 MHz, DMSO-d₆): δ 11.67 (s, 1H), 8.15 (t, 1H), 7.66-7.46 (m, 5H), 7.28 (s, 1H), 7.06 (t, 1H), 6.05 (q, 1H), 2.47 (s, 3H), 1.55 (d, 3H); Chiral analytical SFC: RT=5.96 min, Column: Chiralpak IC-3, (4.6×150 mm, 3 μm), 80% CO₂/Methanol, Flow=3.0 g/min.

### N-(1-(6,7-Difluoro-1-oxo-1,2-dihydroisoquinolin-4-yl)ethyl)-N-methylbenzamide (Compounds 127 & 128)

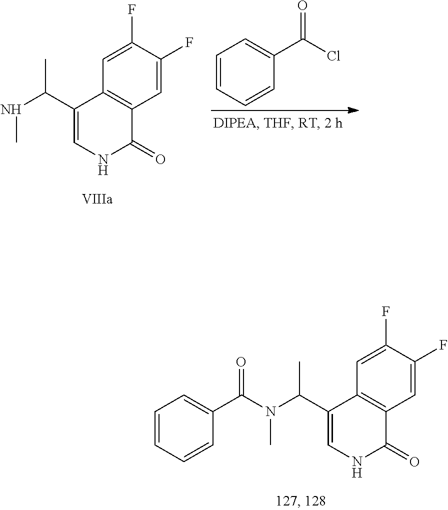

Racemic N-(1-(6,7-difluoro-1-oxo-1,2-dihydroisoquinolin-4-yl)ethyl)-N-methylbenzamide was prepared according to General procedure IV from benzoyl chloride. The enantiomers were subsequently separated by chiral SFC, Column: Chiralpak AS-H (250×30 mm, 5 μm), 80% CO₂/Methanol, Flow rate 100 g/min.

N-(1-(6,7-Difluoro-1-oxo-1,2-dihydroisoquinolin-4-yl)ethyl)-N-methylbenzamide—Enantiomer I (Compound 127), LCMS: m/z found 343.3 [M+H]⁺, RT=1.64 min (Method C); ¹H NMR (400 MHz, DMSO-d₆): δ 11.65 (s, 1H), 8.14 (t, 1H), 7.63 (t, 1H), 7.43 (br s, 3H), 7.30-7.27 (m, 3H), 6.04 (q, 1H), 2.46 (s, 3H), 1.53 (d, 3H); Chiral analytical SFC: RT=2.38 min, Column: Chiralpak AS-3, (4.6×150 mm, 3 μm), 80% CO₂/Methanol, Flow=3.0 g/min.

N-(1-(6,7-Difluoro-1-oxo-1,2-dihydroisoquinolin-4-yl)ethyl)-N-methylbenzamide—Enantiomer II (Compound 128), LCMS: m/z found 343.3 [M+H]⁺, RT=1.64 min (Method C); ¹H NMR (400 MHz, DMSO-d₆): δ 11.65 (s, 1H), 8.14 (t, 1H), 7.63 (t, 1H), 7.43 (br s, 3H), 7.30-7.27 (m, 3H), 6.04 (q, 1H), 2.46 (s, 3H), 1.53 (d, 3H); Chiral analytical SFC: RT=3.28 min, Column: Chiralpak AS-3, (4.6×150 mm, 3 μm), 80% CO₂/Methanol, Flow=3.0 g/min.

### N-(1-(6,7-Difluoro-1-oxo-1,2-dihydroisoquinolin-4-yl)ethyl)-N-methyl-4-(trifluoromethyl) benzamide (Compounds 129 & 130)

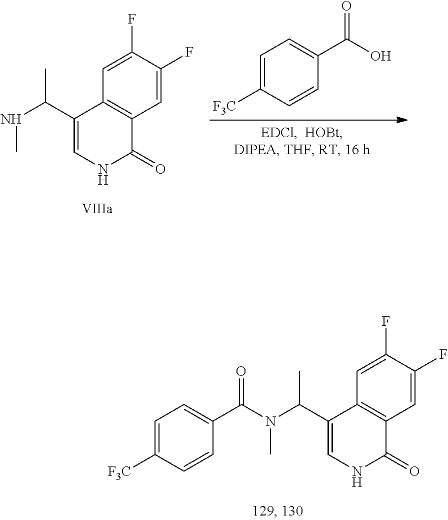 [SMILES: CNC(C)c1c[nH]c(=O)c2cc(F)c(F)cc12 ; O=C(O)c1ccc(C(F)(F)F)cc1 ; CC(c1c[nH]c(=O)c2cc(F)c(F)cc12)N(C)C(=O)c1ccc(C(F)(F)F)cc1]

Racemic N-(1-(6,7-difluoro-1-oxo-1,2-dihydroisoquinolin-4-yl)ethyl)-N-methyl-4-(trifluoromethyl) benzamide was prepared according to General procedure III from 4-(trifluoromethyl)benzoic acid. The enantiomers were subsequently separated by chiral SFC, Column: Chiralpak IC (250×30 mm, 5 μm), 80% CO₂/Methanol, Flow rate 100 g/min.

N-(1-(6,7-Difluoro-1-oxo-1,2-dihydroisoquinolin-4-yl)ethyl)-N-methyl-4-(trifluoromethyl) benzamide—Enantiomer I (Compound 129), LCMS: m/z found 411.3 [M+H]⁺, RT=1.91 min (Method C); ¹H NMR (400 MHz, DMSO-d₆): δ 11.68 (s, 1H), 8.17 (t, 1H), 7.80 (d, 2H), 7.66-7.61 (m, 1H), 7.53 (d, 2H), 7.29 (s, 1H), 6.05 (q, 1H), 2.45 (s, 3H), 1.53 (d, 3H); Chiral analytical SFC: RT=3.52 min, Column: Chiralpak IC-3, (4.6×150 mm, 3 μm), 80% CO₂/Methanol, Flow=3.0 g/min.

N-(1-(6,7-Difluoro-1-oxo-1,2-dihydroisoquinolin-4-yl)ethyl)-N-methyl-4-(trifluoromethyl) benzamide—Enantiomer II (Compound 130), LCMS: m/z found 411.3 [M+H]⁺, RT=1.91 min (Method C); ¹H NMR (400 MHz, DMSO-d₆): δ 11.68 (s, 1H), 8.17 (t, 1H), 7.80 (d, 2H), 7.66-7.61 (m, 1H), 7.53 (d, 2H), 7.29 (s, 1H), 6.05 (q, 1H), 2.45 (s, 3H), 1.53 (d, 3H); Chiral analytical SFC: RT=4.17 min, Column: Chiralpak IC-3, (4.6×150 mm, 3 μm), 80% CO₂/Methanol, Flow=3.0 g/min.

### 4-Chloro-N-(1-(6,7-difluoro-1-oxo-1,2-dihydroisoquinolin-4-yl)ethyl)-N-methylbenzamide (Compounds 131 & 132)

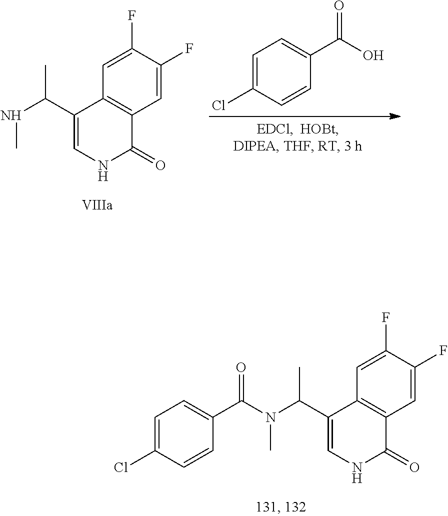 [SMILES: CNC(C)c1c[nH]c(=O)c2cc(F)c(F)cc12 ; O=C(O)c1ccc(Cl)cc1 ; CC(c1c[nH]c(=O)c2cc(F)c(F)cc12)N(C)C(=O)c1ccc(Cl)cc1]

Racemic 4-chloro-N-(1-(6,7-difluoro-1-oxo-1,2-dihydroisoquinolin-4-yl)ethyl)-N-methylbenzamide was prepared according to General procedure III from 4-chlorobenzoic acid. The enantiomers were subsequently separated by chiral SFC, Column: Chiralpak IC (250×30 mm, 5 μm), 70% CO₂/Methanol, Flow rate 100 g/min.

4-Chloro-N-(1-(6,7-difluoro-1-oxo-1,2-dihydroisoquinolin-4-yl)ethyl)-N-methylbenzamide—Enantiomer I (Compound 131), LCMS: m/z found 377.3 [M+H]⁺, RT=1.83 min (Method C); ¹H NMR (400 MHz, DMSO-d₆): δ 11.67 (s, 1H), 8.14 (t, 1H), 7.61 (t, 1H), 7.49 (d, 2H), 7.34 (d, 2H), 7.27 (s, 1H), 6.01 (q, 1H), 2.46 (s, 3H), 1.53 (d, 3H); Chiral analytical SFC: RT=2.38 min, Column: Chiralpak IC-3, (4.6×150 mm, 3 μm), 70% CO₂/Methanol, Flow=3.0 g/min.

4-Chloro-N-(1-(6,7-difluoro-1-oxo-1,2-dihydroisoquinolin-4-yl)ethyl)-N-methylbenzamide—Enantiomer II (Compound 132), LCMS: m/z found 377.3 [M+H]⁺, RT=1.83 min (Method C); ¹H NMR (400 MHz, DMSO-d₆): δ 11.67 (s, 1H), 8.14 (t, 1H), 7.61 (t, 1H), 7.49 (d, 2H), 7.34 (d, 2H), 7.27 (s, 1H), 6.01 (q, 1H), 2.46 (s, 3H), 1.53 (d, 3H); Chiral analytical SFC: RT=5.84 min, Column: Chiralpak IC-3, (4.6×150 mm, 3 μm), 70% CO₂/Methanol, Flow=3.0 g/min.

### N-(1-(6,7-Difluoro-1-oxo-1,2-dihydroisoquinolin-4-yl)ethyl)-3-(difluoromethyl)-4-fluoro-N-methylbenzamide (Compounds 133 & 134)

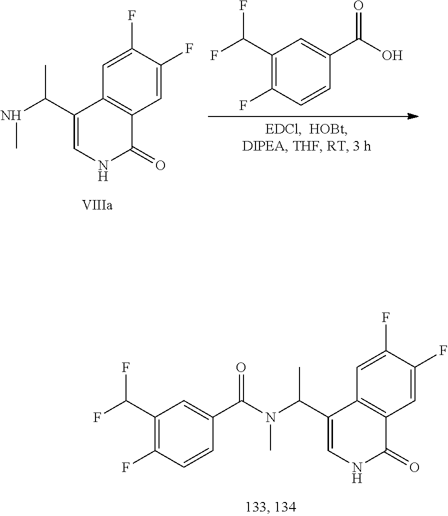

Racemic N-(1-(6,7-difluoro-1-oxo-1,2-dihydroisoquinolin-4-yl)ethyl)-3-(difluoromethyl)-4-fluoro-N-methylbenzamide was prepared according to General procedure III from 3-(difluoromethyl)-4-fluorobenzoic acid. The enantiomers were subsequently separated by chiral SFC, Column: Chiralpak IC (250×30 mm, 5 μm), 80% CO₂/Methanol, Flow rate 100 g/min.

N-(1-(6,7-Difluoro-1-oxo-1,2-dihydroisoquinolin-4-yl)ethyl)-3-(difluoromethyl)-4-fluoro-N-methylbenzamide—Enantiomer I (Compound 133), LCMS: m/z found 411.3 [M+H]⁺, RT=1.77 min (Method C); ¹H NMR (400 MHz, DMSO-d₆): δ 11.67 (s, 1H), 8.14 (t, 1H), 7.61-7.58 (m, 3H), 7.43 (t, 1H), 7.29 (s, 1H), 7.21 (t, 1H), 6.02 (m, 1H), 2.49 (s, 3H), 1.54 (d, 3H); Chiral analytical SFC: RT=3.78 min, Column: Chiralpak IC-3, (4.6×150 mm, 3 μm), 80% CO₂/Methanol, Flow=3.0 g/min.

N-(1-(6,7-Difluoro-1-oxo-1,2-dihydroisoquinolin-4-yl)ethyl)-3-(difluoromethyl)-4-fluoro-N-methylbenzamide—Enantiomer II (Compound 134), LCMS: m/z found 411.3 [M+H]⁺, RT=1.77 min (Method C); ¹H NMR (400 MHz, DMSO-d₆): δ 11.67 (s, 1H), 8.14 (t, 1H), 7.61-7.58 (m, 3H), 7.43 (t, 1H), 7.29 (s, 1H), 7.21 (t, 1H), 6.02 (m, 1H), 2.49 (s, 3H), 1.54 (d, 3H); Chiral analytical SFC: RT=4.47 min, Column: Chiralpak IC-3, (4.6×150 mm, 3 μm), 80% CO₂/Methanol, Flow=3.0 g/min.

### 3-Chloro-N-(1-(6,7-difluoro-1-oxo-1,2-dihydroisoquinolin-4-yl)ethyl)-4-fluoro-N-methylbenzamide (Compounds 135 & 136)

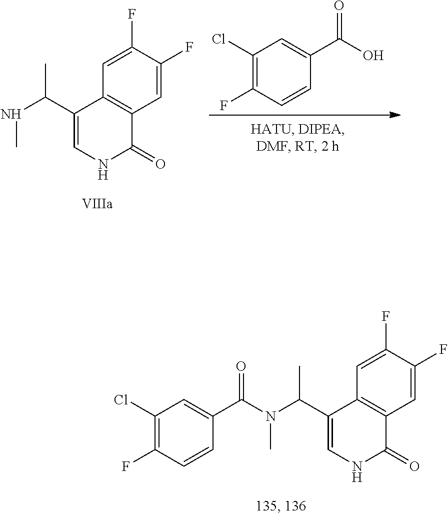

Racemic 3-chloro-N-(1-(6,7-difluoro-1-oxo-1,2-dihydroisoquinolin-4-yl)ethyl)-4-fluoro-N-methylbenzamide was prepared according to General procedure II from 3-chloro-4-fluorobenzoic acid. The enantiomers were subsequently separated by chiral SFC, Column: Chiralpak IC (250×30 mm, 5 μm), 70% CO₂/Methanol, Flow rate 100 g/min.

3-Chloro-N-(1-(6,7-difluoro-1-oxo-1,2-dihydroisoquinolin-4-yl)ethyl)-4-fluoro-N-methylbenzamide—Enantiomer I (Compound 135), LCMS: m/z found 395.2 [M+H]⁺, RT=1.62 min (Method D); ¹H NMR (400 MHz, DMSO-d₆): δ 11.66 (s, 1H), 8.14 (t, 1H), 7.62-7.60 (m, 2H), 7.47 (t, 1H), 7.33 (m, 1H), 7.27 (d, 1H), 6.01 (q, 1H), 2.49 (s, 3H), 1.53 (d, 3H); Chiral analytical SFC: RT=3.62 min, Column: Chiralpak IC-3, (4.6×150 mm, 3 μm), 70% CO₂/Methanol, Flow=3.0 g/min.

3-Chloro-N-(1-(6,7-difluoro-1-oxo-1,2-dihydroisoquinolin-4-yl)ethyl)-4-fluoro-N-methylbenzamide—Enantiomer II (Compound 136), LCMS: m/z found 395.2 [M+H]⁺, RT=1.62 min (Method D); ¹H NMR (400 MHz, DMSO-d₆): δ 11.66 (s, 1H), 8.14 (t, 1H), 7.62-7.60 (m, 2H), 7.47 (t, 1H), 7.33 (m, 1H), 7.27 (d, 1H), 6.01 (q, 1H), 2.49 (s, 3H), 1.53 (d, 3H); Chiral analytical SFC: RT=4.34 min, Column: Chiralpak IC-3, (4.6×150 mm, 3 μm), 70% CO₂/Methanol, Flow=3.0 g/min.

### N-(1-(6,7-Difluoro-1-oxo-1,2-dihydroisoquinolin-4-yl)ethyl)-3,4-difluoro-N-methylbenzamide (Compounds 137 & 138)

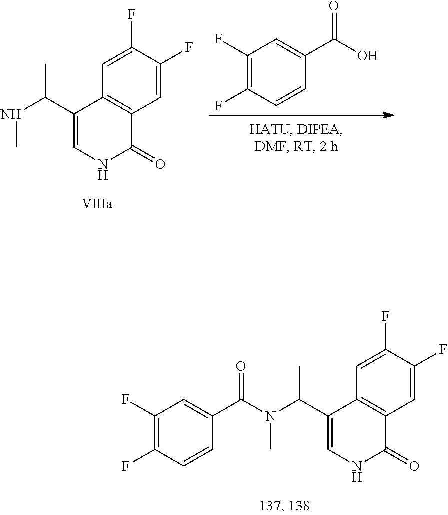

Racemic N-(1-(6,7-difluoro-1-oxo-1,2-dihydroisoquinolin-4-yl)ethyl)-3,4-difluoro-N-methylbenzamide was prepared according to General procedure II from 3,4-difluorobenzoic acid. The enantiomers were subsequently separated by chiral SFC, Column: Chiralpak IC (250×30 mm, 5 μm), 75% CO₂/Methanol, Flow rate 100 g/min.

N-(1-(6,7-Difluoro-1-oxo-1,2-dihydroisoquinolin-4-yl)ethyl)-3,4-difluoro-N-methylbenzamide—Enantiomer I (Compound 137), LCMS: m/z found 379.3 [M+H]⁺, RT=1.55 min (Method D); ¹H NMR (400 MHz, DMSO-d₆): δ 11.66 (s, 1H), 8.14 (t, 1H), 7.61-7.48 (m, 3H), 7.27 (d, 1H), 7.17 (br s, 1H), 6.00 (q, 1H), 2.49 (s, 3H), 1.53 (d, 3H); Chiral analytical SFC: RT=3.58 min, Column: Chiralpak IC-3, (4.6×150 mm, 3 μm), 75% CO₂/Methanol, Flow=3.0 g/min.

N-(1-(6,7-Difluoro-1-oxo-1,2-dihydroisoquinolin-4-yl)ethyl)-3,4-difluoro-N-methylbenzamide—Enantiomer II (Compound 138), LCMS: m/z found 379.3 [M+H]⁺, RT=1.55 min (Method D); ¹H NMR (400 MHz, DMSO-d₆): δ 11.66 (s, 1H), 8.14 (t, 1H), 7.61-7.48 (m, 3H), 7.27 (d, 1H), 7.17 (br s, 1H), 6.00 (q, 1H), 2.49 (s, 3H), 1.53 (d, 3H); Chiral analytical SFC: RT=4.20 min, Column: Chiralpak IC-3, (4.6×150 mm, 3 μm), 75% CO₂/Methanol, Flow=3.0 g/min.

### N-(1-(6,7-Difluoro-1-oxo-1,2-dihydroisoquinolin-4-yl)ethyl)-N,1-dimethyl-1H-indole-5-carboxamide (Compounds 139 & 140)

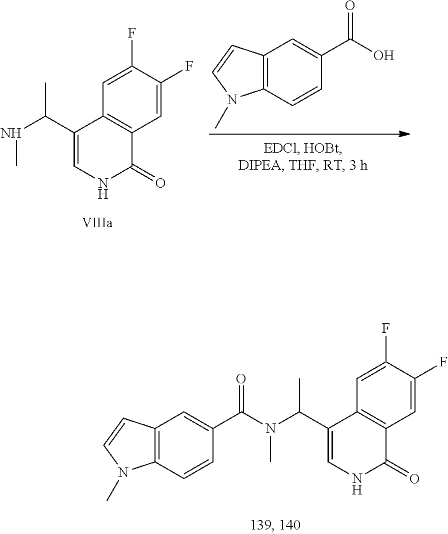

Racemic N-(1-(6,7-difluoro-1-oxo-1,2-dihydroisoquinolin-4-yl)ethyl)-N,1-dimethyl-1H-indole-5-carboxamide was prepared according to General procedure III from 1-methyl-1H-indole-5-carboxylic acid. The enantiomers were subsequently separated by chiral SFC, Column: Chiralpak IC (250×30 mm, 5 μm), 50% CO₂/Methanol, Flow rate 120 g/min.

N-(1-(6,7-difluoro-1-oxo-1,2-dihydroisoquinolin-4-yl)ethyl)-N,1-dimethyl-1H-indole-5-carboxamide—Enantiomer I (Compound 139), LCMS: m/z found 396.3 [M+H]⁺, RT=1.76 min (Method D); ¹H NMR (400 MHz, DMSO-d₆): δ 11.62 (s, 1H), 8.14 (t, 1H), 7.67 (bs, 1H), 7.54 (bs, 1H), 7.47 (d, 1H), 7.39 (d, 1H), 7.27 (s, 1H), 7.12 (s, 1H), 6.47 (d, 1H), (s, 1H), 3.80 (s, 3H), 2.52 (s, 3H), 1.55 (d, 3H); Chiral analytical SFC: RT=3.23 min, Column: Chiralpak IC-3, (4.6×150 mm, 3 m), 50% CO₂/Methanol, Flow=3.0 g/min.

N-(1-(6,7-difluoro-1-oxo-1,2-dihydroisoquinolin-4-yl)ethyl)-N,1-dimethyl-1H-indole-5-carboxamide—Enantiomer II (Compound 140), LCMS: m/z found 396.3 [M+H]⁺, RT=1.76 min (Method D); ¹H NMR (400 MHz, DMSO-d₆): δ 11.62 (s, 1H), 8.14 (t, 1H), 7.67 (bs, 1H), 7.54 (bs, 1H), 7.47 (d, 1H), 7.39 (d, 1H), 7.27 (s, 1H), 7.12 (s, 1H), 6.47 (d, 1H), (s, 1H), 3.80 (s, 3H), 2.52 (s, 3H), 1.55 (d, 3H); Chiral analytical SFC: RT=6.38 min, Column: Chiralpak IC-3, (4.6×150 mm, 3 μm), 50% CO₂/Methanol, Flow=3.0 g/min.

### N-(1-(6,7-Difluoro-1-oxo-1,2-dihydroisoquinolin-4-yl)ethyl)-N,1-dimethyl-1H-indole-6-carboxamide (Compounds 141 & 142)

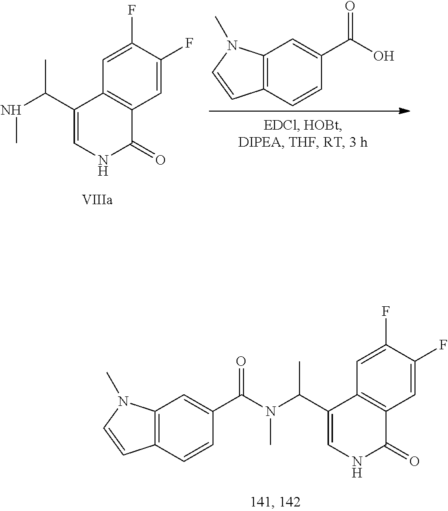

Racemic N-(1-(6,7-difluoro-1-oxo-1,2-dihydroisoquinolin-4-yl)ethyl)-N,1-dimethyl-1H-indole-6-carboxamide was prepared according to General procedure III from 1-methyl-1H-indole-6-carboxylic acid. The enantiomers were subsequently separated by chiral SFC, Column: Chiralpak IC (250×30 mm, 5 μm), 70% CO₂/Methanol, Flow rate 100 g/min.

N-(1-(6,7-Difluoro-1-oxo-1,2-dihydroisoquinolin-4-yl)ethyl)-N,1-dimethyl-1H-indole-6-carboxamide—Enantiomer I (Compound 141), LCMS: m/z found 396.3 [M+H]⁺, RT=1.55 min (Method D); ¹H NMR (400 MHz, DMSO-d₆): δ 11.66 (s, 1H), 8.15 (t, 1H), 7.69 (m, 1H), 7.56 (d, 1H), 7.46-7.43 (m, 2H), 7.27 (s, 1H), 6.93 (s, 1H), 6.44 (s, 1H), 6.06 (s, 1H), 3.80 (s, 3H), 2.54 (s, 3H), 1.17 (d, 3H); Chiral analytical SFC: RT=4.04 min, Column: Chiralpak IC-3, (4.6×150 mm, 3 μm), 75% CO₂/Methanol, Flow=3.0 g/min.

N-(1-(6,7-Difluoro-1-oxo-1,2-dihydroisoquinolin-4-yl)ethyl)-N,1-dimethyl-1H-indole-6-carboxamide—Enantiomer II (Compound 142), LCMS: m/z found 396.3 [M+H]⁺, RT=1.55 min (Method D); ¹H NMR (400 MHz, DMSO-d₆): δ 11.66 (s, 1H), 8.15 (t, 1H), 7.69 (m, 1H), 7.56 (d, 1H), 7.46-7.43 (m, 2H), 7.27 (s, 1H), 6.93 (bs, 1H), 6.44 (s, 1H), 6.06 (bs, 1H), 3.80 (s, 3H), 2.54 (s, 3H), 1.17 (d, 3H); Chiral analytical SFC: RT=5.40 min, Column: Chiralpak IC-3, (4.6×150 mm, 3 μm), 75% CO₂/Methanol, Flow=3.0 g/min.

### N1-Cyclopropyl-N2-(1-(6,7-difluoro-1-oxo-1,2-dihydroisoquinolin-4-yl)ethyl)-N2-methyloxalamide (Compound 143)

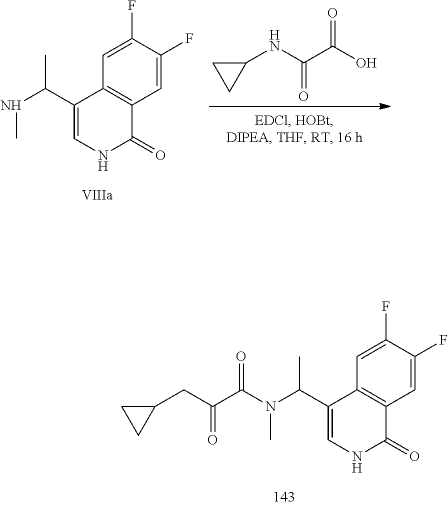

Racemic N1-cyclopropyl-N2-(1-(6,7-difluoro-1-oxo-1,2-dihydroisoquinolin-4-yl)ethyl)-N2-methyloxalamide was prepared according to General procedure III from 2-(cyclopropylamino)-2-oxoacetic acid. The product was purified by flash chromatography (silica gel, 0-3% MeOH in DCM gradient). LCMS: m/z found 350.3 [M+H]⁺, RT=1.45 min (Method D); ¹H NMR (400 MHz, DMSO-d₆): δ 11.68 (s, 1H), 8.78 (d, 1H), 8.14-8.10 (m, 1H), 7.50-7.45 (m, 1H), 7.27-7.22 (m, 1H), 5.79 (q, 1H), 2.71-2.69 (m, 1H), 2.54 (s, 3H), 1.44 (d, 3H), 0.71 (d, 2H), 0.65 (d, 2H).

### N1-(3-Chloro-4-fluorophenyl)-N2-(1-(6,7-difluoro-1-oxo-1,2-dihydroisoquinolin-4-yl)ethyl)-N2-methyloxalamide (Compound 144)

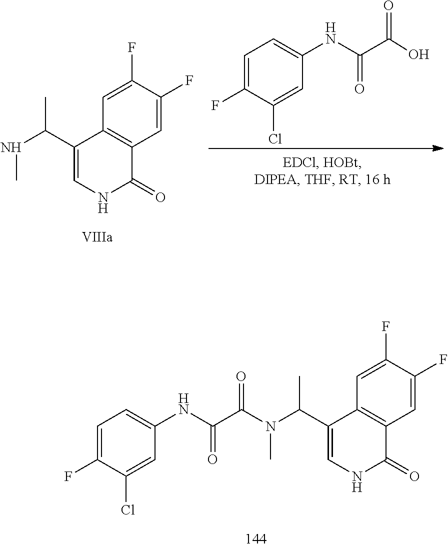

Racemic N1-(3-chloro-4-fluorophenyl)-N2-(1-(6,7-difluoro-1-oxo-1,2-dihydroisoquinolin-4-yl)ethyl)-N2-methyloxalamide was prepared according to General procedure III from 2-((3-chloro-4-fluorophenyl)amino)-2-oxoacetic acid. The product was purified by flash chromatography (silica gel, 0-3% MeOH in DCM gradient). LCMS: m/z found 438.3 [M+H]⁺, RT=1.99 min (Method D); ¹H NMR (400 MHz, DMSO-d₆): δ 11.72-11.68 (m, 1H), 11.27-11.04 (m, 1H), 8.16-8.09 (m, 1H), 8.00-7.93 (dd, 1H), 7.62-7.39 (m, 3H), 7.31 (d, 1H), 5.85 (d, 1H), 2.66 (s, 3H), 1.51 (d, 3H).

### 1-(6,7-Difluoro-1-methoxyisoquinolin-4-yl)ethan-1-one (Va)

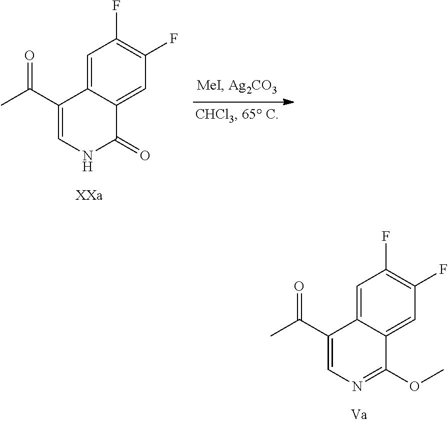 [SMILES: CC(=O)c1c[nH]c(=O)c2cc(F)c(F)cc12 ; COc1ncc(C(C)=O)c2cc(F)c(F)cc12]

A mixture of 2.5 mL (40.3 mmol) of iodomethane, 7.4 g (26.9 mmol) of silver carbonate and 3.0 g (13.4 mmol) of 4-acetyl-6,7-difluoro-2H-isoquinolin-1-one (XXa) in 70 mL of chloroform in a sealed tube was heated at 65° C. for 24 h. The mixture was allowed to cool to room temperature and diluted with 32 mL of ethyl acetate and 8 mL of acetonitrile. The mixture was then filtered through a pad of CELITE© and the pad was washed with 100 mL of ethyl acetate. The combined filtrate was evaporated in vacuo and the residue was purified by flash chromatography (SiO₂, eluting with a gradient of 0-50% ethyl acetate/hexanes) to provide 2.1 g (8.7 mmol, 65% yield) of 1-(6,7-difluoro-1-methoxyisoquinolin-4-yl)ethan-1-one (Va). ¹H NMR (400 MHz, CDCl₃) δ 9.01 (m, 1H), 8.74 (s, 1H), 8.02 (m, 1H), 4.19 (s, 3H), 2.70 (s, 3H).

### (S)-N-((R)-1-(6,7-Difluoro-1-methoxyisoquinolin-4-yl)ethyl)-2-methylpropane-2-sulfinamide (XIIIa)

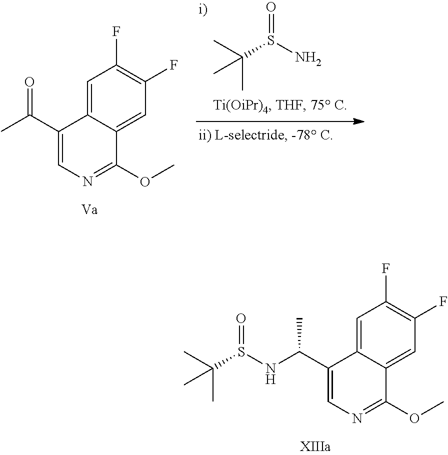

To a mixture of 1.83 g (7.72 mmol, 1.0 eq.) of 1-(6,7-difluoro-1-methoxy-4-isoquinolyl)ethanone (Va) and 1.22 g (10.03 mmol, 1.3 eq.) of (S)-2-methylpropane-2-sulfinamide in 2.2 mL of anhydrous THF in a sealed tube was added 4.57 mL (15.44 mmol, 2.0 eq.) of tetraisopropoxytitanium and the mixture was heated at 80° C. for 26 h. The mixture was allowed to cool to room temperature and diluted with 35 mL of THF. The mixture was further cooled to −78° C. under a nitrogen atmosphere and 7.73 mL (7.73 mmol, 1.0 eq.) of a 1 M solution of L-selectride in THF was added and the mixture was stirred at −78° C. for 90 minutes. An additional portion of 0.3 mL (0.3 mmol) of a 1 M solution of L-selectride in THF was added and stirring was continued for a further 45 min. The mixture then was diluted with 10 mL of methanol and the cooling bath was removed. Upon warming to room temperature, the mixture was diluted with 70 mL of 1:6 v/v acetonitrile/ethyl acetate and added to 10 mL of a rapidly stirred brine solution. After stirring for 10 minutes, the mixture was filtered through CELITE® and the pad was washed with 20 mL of ethyl acetate. The combined filtrate was evaporated in vacuo and the major diastereoisomer was isolated by flash chromatography (SiO₂, eluting with a linear gradient of 0-100% of ethyl acetate/methylene chloride) to provide 1.70 g (4.96 mmol, 64% yield) of diastereomerically pure (S)-N-((R)-1-(6,7-difluoro-1-methoxyisoquinolin-4-yl)ethyl)-2-methylpropane-2-sulfinamide (XIIIa). ¹H NMR (400 MHz, CDCl₃) δ 7.98-8.08 (m, 2H), 7.83 (m, 1H), 4.91-5.02 (m, 1H), 4.11 (s, 3H), 3.34 (d, 1H), 1.73 (d, 3H), 1.21 (s, 9H).

The absolute configuration of the α-methyl substituent of XIIIa has been inferred from previous X-ray crystallographic studies, wherein 1-(1-methoxy-4-isoquinolyl)ethanone was used as the ketone substrate in place of Va, under identical conditions as those described herein, as detailed in WO 2020123674.

### (S)-N-((R)-1-(6,7-Difluoro-1-methoxyisoquinolin-4-yl)ethyl)-N,2-dimethylpropane-2-sulfinamide (XIVa)

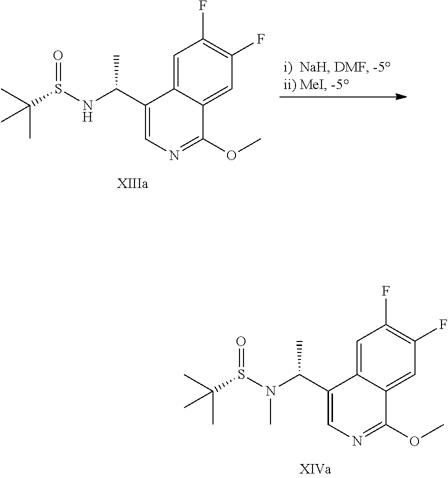 [SMILES: COc1ncc([C@@H](C)N[S@@](=O)C(C)(C)C)c2cc(F)c(F)cc12 ; COc1ncc([C@@H](C)N(C)[S@@](=O)C(C)(C)C)c2cc(F)c(F)cc12]

To a solution of 1.47 g (4.29 mmol, 1.0 eq.) of diastereomerically pure (S)-N-[(R)-1-(6,7-difluoro-1-methoxy-4-isoquinolyl)ethyl]-2-methyl-propane-2-sulfinamide (XIIIa) in 22 mL of anhydrous DMF under a nitrogen atmosphere at −5° C. was added 0.31 g (7.73 mmol, 1.8 eq.) of a 60% dispersion of sodium hydride in mineral oil. The mixture was stirred at −5° C. for 25 minutes and 0.53 mL (8.59 mmol, 2.0 eq.) of iodomethane was added. The mixture was stirred at −5° C. for a further 20 min and then quenched by the addition of 30 mL water. The mixture was then extracted with 2×50 mL of ethyl acetate and the combined organic extracts were washed with 2×30 mL of water followed by 30 mL of brine, dried (Na₂SO₄), filtered and the solvent was removed in vacuo. The residue was purified by flash chromatography (SiO₂, eluting with a gradient of 0% to 5% MeOH/DCM) to provide 1.39 (3.90 mmol, 90% yield) of (S)-N-[(R)-1-(6,7-difluoro-1-methoxy-4-isoquinolyl)ethyl]-N,2-dimethyl-propane-2-sulfinamide (XIVa). LCMS: m/z found 357.2 [M+H]⁺, RT=4.73 min (Method A). ¹H NMR (400 MHz, CDCl₃) δ 7.97-8.07 (m, 2H), 7.76 (m, 1H), 5.00-5.11 (m, 1H), 4.12 (s, 3H), 2.37 (s, 3H), 1.75 (d, 3H), 1.24 (s, 9H).

### (R)-6,7-Difluoro-4-(1-(methylamino)ethyl)isoquinolin-1(2H)-one hydrochloride salt ((R)-VIIIa)

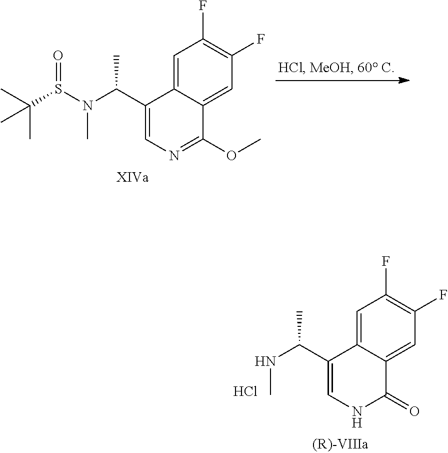

To a solution of 1.39 g (3.90 mmol, 1.0 eq.) of (S)-N-[1-(6,7-difluoro-1-methoxy-4-isoquinolyl)ethyl]-N,2-dimethyl-propane-2-sulfinamide (XIVa) in 14 mL of methanol in a sealed tube was added 13 mL (39.0 mmol, 10 eq.) of a 3 M solution of HCl in methanol and the mixture was heated at 60° C. for 15 h. The mixture was allowed to cool to room temperature and the volatiles were removed in vacuo. The residue was subsequently triturated with diethyl ether (2×10 mL) to provide 1.09 g of (R)-6,7-difluoro-4-(1-(methylamino)ethyl)isoquinolin-1(2H)-one hydrochloride salt ((R)-VIIIa). ¹H NMR (400 MHz, Methanol-d₄) δ 8.22 (m, 1H), 7.94 (m, 1H), 7.51 (s, 1H), 4.81-4.92 (m, 1H), 2.72 (s, 3H), 1.73 (d, 3H).

### (R)-N-(1-(6,7-Difluoro-1-oxo-1,2-dihydroisoquinolin-4-yl)ethyl)-N-methyl-4,5,6,7-tetrahydro-1H-indole-2-carboxamide (Compound 34)

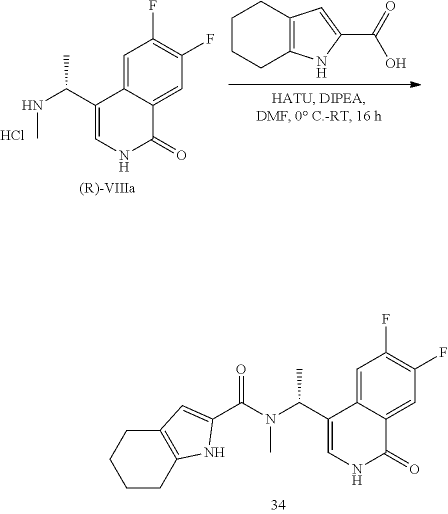

HATU (43 mg, 0.11 mmol) and N,N-diisopropylethylamine (44 μL, 0.25 mmol) were added to a solution of 4,5,6,7-tetrahydro-1H-indole-2-carboxylic acid (17 mg, 0.10 mmol) in 0.75 mL of DMF at 0° C. After 25 minutes, a solution of (R)-6,7-difluoro-4-(1-(methylamino)ethyl)isoquinolin-1(2H)-one hydrochloride salt ((R)-VIIIa, 28 mg, 0.10 mmol) in 0.75 mL of DMF was added. The mixture was allowed to warm to room temperature and stirred for 16 h. The reaction mixture was then diluted with 30 mL of ethyl acetate and washed with 0.1 M HCl (2×8 mL) followed by saturated sodium bicarbonate solution (8 mL). The organics were dried (Na₂SO₄), filtered and the solvent was removed in vacuo. The residue was purified by flash chromatography (silica gel, eluting with a linear gradient of 15-100% ethyl acetate/hexanes) to provide (R)-N-(1-(6,7-difluoro-1-oxo-1,2-dihydroisoquinolin-4-yl)ethyl)-N-methyl-4,5,6,7-tetrahydro-1H-indole-2-carboxamide. LCMS: m/z found 386.3 [M+H]⁺, RT=4.23 min (Method A); ¹H NMR (400 MHz, CDCl₃) δ 10.66 (s, 1H), 9.27 (s, 1H), 8.21 (dd, 1H), 7.71 (s, 1H), 7.22-7.15 (m, 1H), 6.34-6.26 (m, 2H), 2.90 (s, 3H), 2.66 (t, 2H), 2.49 (t, 2H), 1.89-1.69 (m, 3H), 1.53 (d, 3H).

### (R)-N-(1-(6,7-Difluoro-1-oxo-1,2-dihydroisoquinolin-4-yl)ethyl)-N-methylindolizine-6-carboxamide (Compound 185)

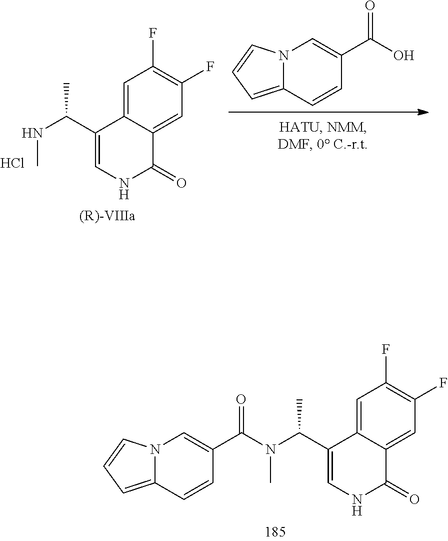

Enantiomerically pure (R)-N-(1-(6,7-difluoro-1-oxo-1,2-dihydroisoquinolin-4-yl)ethyl)-N-methylindolizine-6-carboxamide was synthesized in a similar manner as described above (General procedure II, except employing N-methyl morpholine as the base), starting from (R)-6,7-difluoro-4-(1-(methylamino)ethyl)isoquinolin-1(2H)-one hydrochloride salt ((R)-VIIIa) and indolizine-6-carboxylic acid. LCMS: m/z found 382.1 [M+H]⁺, RT=3.85 min (Method A); ¹H NMR (400 MHz, DMSO-d₆): δ 11.70 (d, 1H), 8.48 (s, 1H), 8.15 (dd, 1H), 7.60 (s, 2H), 7.43 (d, 1H), 7.29 (d, 1H), 6.80 (dd, 1H), 6.61 (d, 1H), 6.44 (d, 1H), 5.99 (s, 1H), 2.63 (s, 3H), 1.54 (d, 3H).

### (R)-2-Chloro-N-(1-(6,7-difluoro-1-oxo-1,2-dihydroisoquinolin-4-yl)ethyl)-N-methyl-4H-thieno[3,2-b]pyrrole-5-carboxamide (Compound 186)

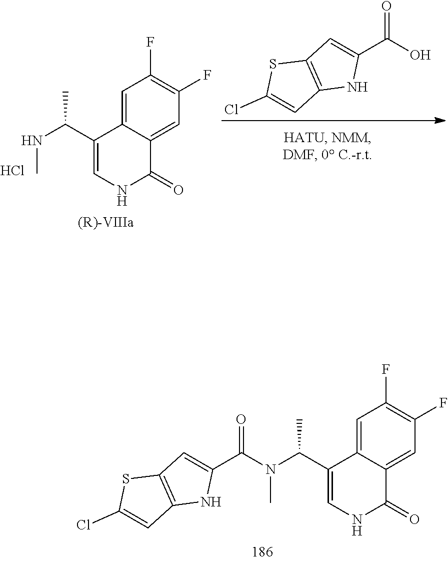

Enantiomerically pure (R)-2-chloro-N-(1-(6,7-difluoro-1-oxo-1,2-dihydroisoquinolin-4-yl)ethyl)-N-methyl-4H-thieno[3,2-b]pyrrole-5-carboxamide was synthesized in a similar manner as described above, starting from (R)-6,7-difluoro-4-(1-(methylamino)ethyl)isoquinolin-1(2H)-one hydrochloride salt ((R)-VIIIa) and 2-chloro-4H-thieno[3,2-b]pyrrole-5-carboxylic acid. LCMS: m/z found 422.0 [M+H]⁺, RT=4.55 min (Method A); ¹H NMR (400 MHz, DMSO-d₆): δ 11.98 (s, 1H), 11.71 (d, 1H), 8.12 (dd, 1H), 7.62 (s, 1H), 7.31 (d, 1H), 7.08 (s, 1H), 6.87 (d, 1H), 6.07 (d, 1H), 2.88 (s, 3H), 1.50 (d, 3H).

### (R)-N-(1-(6,7-Difluoro-1-oxo-1,2-dihydroisoquinolin-4-yl)ethyl)-N-methylindolizine-7-carboxamide (Compound 187)

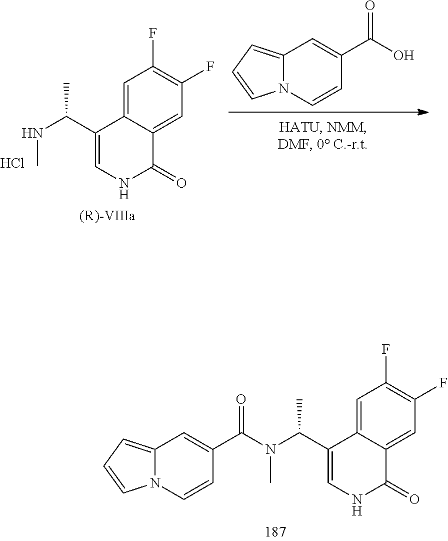

Enantiomerically pure (R)-N-(1-(6,7-difluoro-1-oxo-1,2-dihydroisoquinolin-4-yl)ethyl)-N-methylindolizine-7-carboxamide was synthesized in a similar manner as described above, starting from (R)-6,7-difluoro-4-(1-(methylamino)ethyl)isoquinolin-1(2H)-one hydrochloride salt ((R)-VIIIa) and indolizine-7-carboxylic acid. LCMS: m/z found 382.1 [M+H]⁺, RT=3.67 min (Method A); ¹H NMR (400 MHz, DMSO-d₆): δ 11.69 (d, 1H), 8.28 (d, 1H), 8.14 (dd, 1H), 7.63-7.59 (m, 2H), 7.48 (s, 1H), 7.28 (d, 1H), 6.80 (dd, 1H), 6.51 (dd, 2H), 5.99 (s, 1H), 2.61 (s, 3H), 1.54 (d, 3H).

### Synthesis of 7,8-difluoroisoquinolin-1(2H)-one (IIb)

### 1-(2,3-Difluorophenyl)-N-(2,2-dimethoxyethyl)methanamine

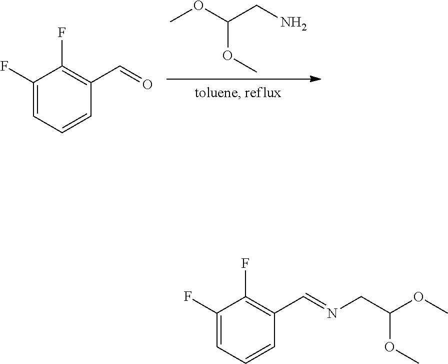

To a solution of 25.0 g (176.1 mmol, 1.0 eq.) of 2,3-difluorobenzaldehyde in 200 mL of toluene in an apparatus equipped with a Dean-Stark trap was added 18.5 g (176.1 mmol, 1.0 eq.) of 2,2-dimethoxyethanamine and the mixture was heated to azeotropic reflux for 16 h. The mixture was allowed to cool to room temperature and the solvent was removed in vacuo to provide 35 g of crude (E)-1-(2,3-difluorophenyl)-N-(2,2-dimethoxyethyl)methanimine which was used without further purification. ¹H NMR (400 MHz, CDCl₃): δ 8.57 (s, 1H), 7.72-7.77 (m, 1H), 7.18-7.25 (m, 1H), 7.07-7.13 (m, 1H), 4.69 (t, 1H), 3.81 (d, 2H), 3.43 (s, 6H).

### 7,8-Difluoroisoquinoline

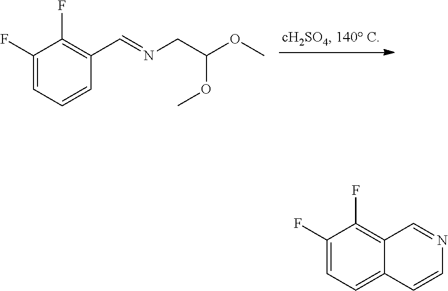 [SMILES: COC(C/N=C/c1cccc(F)c1F)OC ; Fc1ccc2ccncc2c1F]

A mixture of 35 g of (E)-1-(2,3-difluorophenyl)-N-(2,2-dimethoxyethyl)methanimine and 250 mL of chilled conc. H₂SO₄ was stirred at 140° C. for 30 min. The mixture was allowed to cool to room temperature and slowly poured into 600 mL of ice-cold water. The mixture was then filtered through CELITE® and the pad was washed with 100 mL of water. The filtrate was washed with methylene chloride (2×500 mL) and the aqueous layer was basified with 500 mL of 10 M aqueous sodium hydroxide solution and extracted with methylene chloride (3×500 mL). The combined organic extracts were dried (Na₂SO₄), filtered and the solvent was removed in vacuo to provide 5.0 g (30.30 mmol, 17% yield from 2,3-difluorobenzaldehyde) of 7,8-difluoroisoquinoline. LCMS: m/z found 166.0 [M+H]⁺, ¹H NMR (400 MHz, DMSO-d₆): δ 9.51 (s, 1H), 8.63 (d, 1H), 7.89-7.97 (m, 3H).

### 7,8-Difluoroisoquinoline 2-oxide

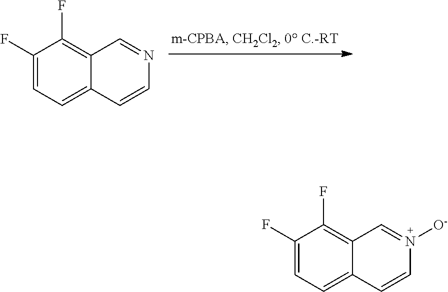

To a stirred solution of 5.0 g (30.3 mmol, 1.0 eq.) of 7,8-difluoroisoquinoline in 100 mL of methylene chloride at 0° C. was added 10.4 g (60.6 mmol, 2.0 eq.) of m-chloroperoxybenzoic acid portion-wise over approximately 15 min. The mixture was allowed to warm to room temperature and stirred for 7 h. The reaction mixture was re-cooled to 0° C. and quenched by the addition of 200 mL of saturated sodium bicarbonate solution and then extracted with 10% methanol in methylene chloride (3×200 mL). The combined organic extracts were washed with 50 mL of brine, dried (Na₂SO₄), filtered and the solvent was removed in vacuo. The residue was triturated with n-pentane (2×100 mL), filtered, and dried under high vacuum to provide 4.5 g (24.8 mmol, 90% yield) of 7,8-difluoroisoquinoline 2-oxide. LCMS: m/z found 181.9 [M+H]⁺, H NMR (400 MHz, DMSO-d₆): δ 8.89 (s, 1H), 8.21-8.24 (m, 1H), 8.03-8.06 (m, 1H), 7.87-7.90 (m, 1H), 7.68-7.76 (m, 1H).

### 7,8-Difluoroisoquinolin-1(2H)-one (IIb)

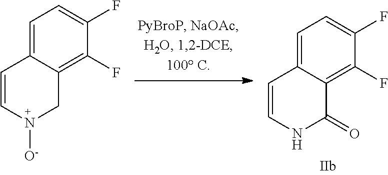 [SMILES: [O-]N1C=Cc2ccc(F)c(F)c2C1 ; O=c1[nH]ccc2ccc(F)c(F)c12]

To a suspension of 2.5 g (13.8 mmol, 1.0 eq.) of 7,8-difluoroisoquinoline 2-oxide in 25 mL of 1,2-dichloroethane was added 3.4 g (41.4 mmol, 3.0 eq.) of sodium acetate followed by 12.9 g (27.6 mmol, 2.0 eq.) of bromotripyrrolidinophosphonium hexafluorophosphate (PyBroP) and 3.7 mL (207.2 mmol, 15 eq.) of water and the mixture was heated at 100° C. for 12 h. The mixture was allowed to cool to room temperature and diluted with 30 mL of methylene chloride and 50 mL of water. The resulting suspension was filtered and the solid was dissolved in 200 mL of 50% methanol in methylene chloride, dried (Na₂SO₄), filtered and the solvent was removed in vacuo to provide 1.3 g (7.2 mmol, 52% yield) of 7,8-difluoroisoquinolin-1(2H)-one (IIb). LCMS: m/z found 182.1 [M+H]⁺, ¹H NMR (400 MHz, DMSO-d₆): δ 11.34 (bs, 1H), 7.74-7.82 (m, 1H), 7.49-7.53 (m, 1H), 7.14-7.17 (m, 1H), 6.53-6.54 (m, 1H).

The above detailed reaction sequence was performed on multiple batches with consistent results.

### 4-Bromo-7,8-difluoroisoquinolin-1(2H)-one (IIIb)

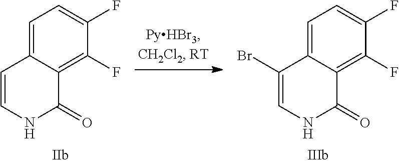 [SMILES: O=c1[nH]ccc2ccc(F)c(F)c12 ; O=c1[nH]cc(Br)c2ccc(F)c(F)c12]

To a suspension of 3.8 g (21.0 mmol, 1.0 eq.) of 7,8-difluoroisoquinolin-1(2H)-one (IIb) in 10 mL of methylene chloride was added 5.26 g (16.6 mmol, 0.8 eq.) of pyridinium bromide perbromide and the mixture was stirred at room temperature for 3 h. The reaction was then quenched by the addition of 100 mL of saturated sodium bicarbonate solution and the solvent was removed in vacuo. The residue was suspended in 150 mL of water, filtered and the solids were washed with 60 mL of n-pentane and dried under high vacuum to provide 4.0 g (15.38 mmol, 93% yield) of 4-bromo-7,8-difluoroisoquinolin-1(2H)-one (IIb). LCMS: m/z found 260.0/262.0 [M+H]⁺, ¹H NMR (400 MHz, DMSO-d₆): δ 11.67 (bs, 1H), 7.90-7.97 (m, 1H), 7.57-7.63 (m, 2H).

### 4-Acetyl-7,8-difluoroisoquinolin-1(2H)-one (XXb)

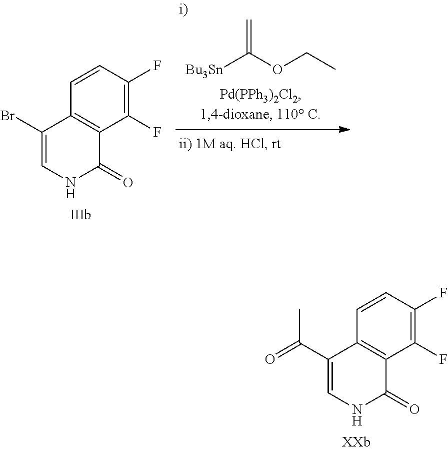

To a solution of 3.0 g (11.58 mmol, 1.0 eq.) of 4-bromo-7,8-difluoroisoquinolin-1(2H)-one (IIIb) in 30 mL of 1,4-dioxane was added 10.48 g (28.95 mmol, 2.5 eq.) of tributyl(1-ethoxyvinyl)stannane. The mixture was purged with nitrogen gas for 5 min and 0.81 g (1.15 mmol, 0.1 eq.) of Pd(PPh₃)₂Cl₂ was added, and then heated to 110° C. for 16 h. The reaction mixture was allowed to cool to room temperature and 30 mL of 1 M aqueous HCl was added and stirring was continued for an additional 3 h. The reaction mixture was then basified with 50 mL of saturated sodium bicarbonate solution and extracted with ethyl acetate (3×200 mL). The combined organic extracts were washed with 100 mL of water, 100 mL of brine, dried (Na₂SO₄), filtered and the solvent was removed in vacuo. The residue was triturated with 60 mL of n-pentane, filtered the solids were dried under high vacuum to provide 2.3 g of 4-acetyl-7,8-difluoroisoquinolin-1(2H)-one (XXb). LCMS: m/z found 224.0 [M+H]⁺.

The above reaction sequence was performed on multiple batches with consistent results.

### 7,8-Difluoro-4-(1-(methylamino)ethyl)isoquinolin-1(2H)-one (VIIIf)

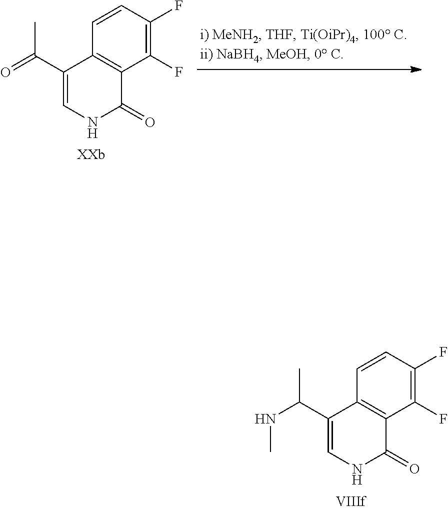 [SMILES: CC(=O)c1c[nH]c(=O)c2c(F)c(F)ccc12 ; CNC(C)c1c[nH]c(=O)c2c(F)c(F)ccc12]

7,8-Difluoro-4-(1-(methylamino)ethyl)isoquinolin-1(2H)-one (VIIIf) was synthesized according to General Procedure I from 4-acetyl-7,8-difluoroisoquinolin-1(2H)-one (XXb) and a 2.0 M solution of methyl amine in THF. LCMS: m/z found 239.1 [M+H]⁺.

### N-(1-(7,8-Difluoro-1-oxo-1,2-dihydroisoquinolin-4-yl)ethyl)-N-methyl-1,4,5,6-tetrahydrocyclopenta[b]pyrrole-2-carboxamide (Compounds 57 & 58)

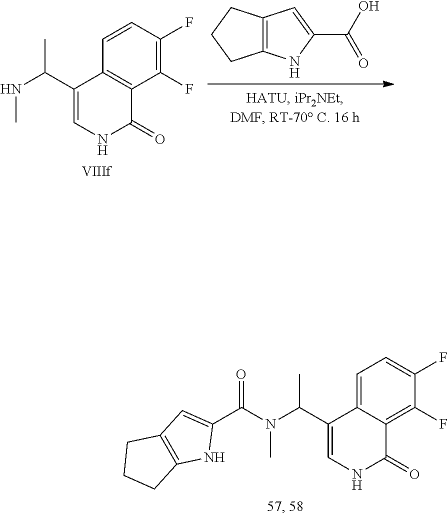

Racemic N-(1-(7,8-difluoro-1-oxo-1,2-dihydroisoquinolin-4-yl)ethyl)-N-methyl-1,4,5,6-tetrahydrocyclopenta[b]pyrrole-2-carboxamide was synthesized in a similar manner as described above from 7,8-difluoro-4-(1-(methylamino)ethyl)isoquinolin-1(2H)-one (VIIIf) and 1,4,5,6-tetrahydrocyclopenta[b]pyrrole-2-carboxylic acid. The enantiomers were subsequently separated by chiral SFC, Column: Chiralpak IC (250×30 mm, 5 m), 70% CO₂/(30 mM ammonia in methanol), Flow rate 90 g/min. Enantiomer I was further purified by preparative HPLC (Column: Kromasil (150×25 mm, 10 μm), Mobile phase A: 10 mM ammonium bicarbonate in water, Mobile phase B: Acetonitrile; Method T/% B: 1/50, 11/60, 11.1/100, 13/100, 13.1/50, 15/50).

N-(1-(7,8-Difluoro-1-oxo-1,2-dihydroisoquinolin-4-yl)ethyl)-N-methyl-1,4,5,6-tetrahydrocyclopenta[b]pyrrole-2-carboxamide—Enantiomer I (Compound 57), LCMS: m/z found 372.2 [M+H]⁺, RT=3.63 min (Method A); ¹H NMR (400 MHz, DMSO-d₆): δ 11.51 (bs, 1H), 11.16 (bs, 1H), 7.78-7.71 (m, 1H), 7.38-7.36 (m, 1H), 7.20 (s, 1H), 6.24 (s, 1H), 6.02-5.98 (m, 1H), 2.76 (s, 3H), 2.63-2.59 (m, 2H), 2.49-2.46 (m, 2H), 2.34-2.27 (m, 2H), 1.44 (d, 3H); Chiral analytical SFC: RT=7.37 min, Column: Chiralpak IC-3, (4.6×150 mm, 3 μm), 70% CO₂/Methanol, Flow=3.0 g/min.

N-(1-(7,8-Difluoro-1-oxo-1,2-dihydroisoquinolin-4-yl)ethyl)-N-methyl-1,4,5,6-tetrahydrocyclopenta[b]pyrrole-2-carboxamide—Enantiomer II (Compound 58), LCMS: m/z found 372.2 [M+H]⁺, RT=3.63 min (Method A); ¹H NMR (400 MHz, DMSO-d₆): δ 11.51 (br s, 1H), 11.16 (br s, 1H), 7.78-7.71 (m, 1H), 7.38-7.36 (m, 1H), 7.20 (s, 1H), 6.24 (s, 1H), 6.02-5.98 (m, 1H), 2.76 (s, 3H), 2.63-2.59 (m, 2H), 2.49-2.46 (m, 2H), 2.34-2.27 (m, 2H), 1.44 (d, 3H); Chiral analytical SFC: RT=8.78 min, Column: Chiralpak IC-3, (4.6×150 mm, 3 μm), 70% CO₂/Methanol, Flow=3.0 g/min.

### N-(1-(7,8-Difluoro-1-oxo-1,2-dihydroisoquinolin-4-yl)ethyl)-N-methylindolizine-2-carboxamide (Compounds 71 & 72)

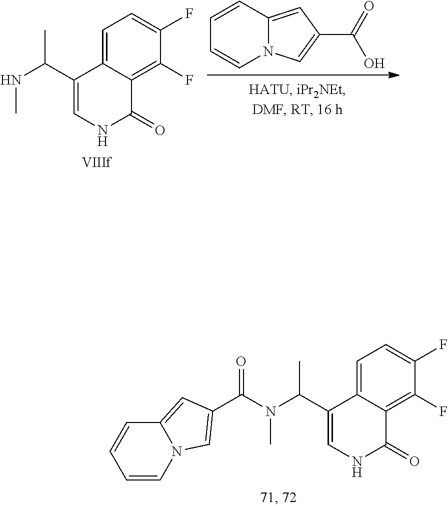

Racemic N-(1-(7,8-difluoro-1-oxo-1,2-dihydroisoquinolin-4-yl)ethyl)-N-methylindolizine-2-carboxamide was synthesized in a similar manner as described above, from 7,8-difluoro-4-(1-(methylamino)ethyl)isoquinolin-1(2H)-one (VIIIf) and indolizine-2-carboxylic acid. The enantiomers were subsequently separated by chiral SFC, Column: Chiralpak IC (250×30 mm, 5 μm), 60% CO₂/Methanol, Flow rate 120 g/min.

N-(1-(7,8-Difluoro-1-oxo-1,2-dihydroisoquinolin-4-yl)ethyl)-N-methylindolizine-2-carboxamide—Enantiomer I (Compound 71), LCMS: m/z found 382.2 [M+H]⁺, RT=3.31 min (Method A); ¹H NMR (400 MHz, DMSO-d₆): δ 11.50 (bs, 1H), 8.20 (d, 1H), 7.83 (s, 2H), 7.38 (d, 2H), 7.21 (s, 1H), 6.74-6.70 (t, 1H), 6.60-6.57 (t, 2H), 6.04 (bs, 1H), 2.75 (s, 3H), 1.49 (s, 3H); Chiral analytical SFC: RT=5.89 min, Column: Chiralpak IC-3, (4.6×150 mm, 3 μm), 60% CO₂/(0.2% DEA in methanol), Flow=3.0 g/min.

N-(1-(7,8-Difluoro-1-oxo-1,2-dihydroisoquinolin-4-yl)ethyl)-N-methylindolizine-2-carboxamide—Enantiomer II (Compound 72), LCMS: m/z found 382.2 [M+H]⁺, RT=3.31 min (Method A); ¹H NMR (400 MHz, DMSO-d₆): δ 11.50 (bs, 1H), 8.20 (d, 1H), 7.83 (s, 2H), 7.38 (d, 2H), 7.21 (s, 1H), 6.74-6.70 (t, 1H), 6.60-6.57 (t, 2H), 6.04 (bs, 1H), 2.75 (s, 3H), 1.49 (s, 3H); Chiral analytical SFC: RT=8.81 min, Column: Chiralpak IC-3, (4.6×150 mm, 3 μm), 60% CO₂/(0.2% DEA in methanol), Flow=3.0 g/min.

### N-(1-(7,8-Difluoro-1-oxo-1,2-dihydroisoquinolin-4-yl)ethyl)-8-fluoro-N-methylindolizine-2-carboxamide (Compounds 81 & 82)

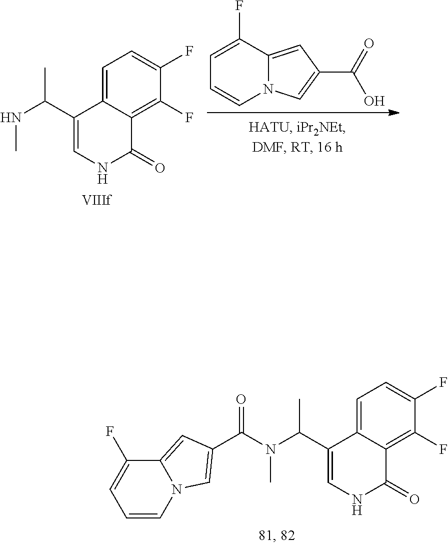

Racemic N-(1-(7,8-difluoro-1-oxo-1,2-dihydroisoquinolin-4-yl)ethyl)-8-fluoro-N-methylindolizine-2-carboxamide was synthesized in a similar manner as described above, from 7,8-difluoro-4-(1-(methylamino)ethyl)isoquinolin-1(2H)-one (VIIIf) and 8-fluoroindolizine-2-carboxylic acid. The enantiomers were subsequently separated by chiral SFC, Column: Chiralpak IC (250×30 mm, 5 μm), 60% CO₂/Methanol, Flow rate 120 g/min.

N-(1-(7,8-Difluoro-1-oxo-1,2-dihydroisoquinolin-4-yl)ethyl)-8-fluoro-N-methylindolizine-2-carboxamide—Enantiomer I (Compound 81), LCMS: m/z found 400.2 [M+H]⁺, RT=3.52 min (Method A); ¹H NMR (400 MHz, DMSO-d₆): δ 11.54 (bs, 1H), 8.11 (bs, 1H), 7.99 (s, 1H), 7.88-7.82 (m, 1H), 7.41 (s, 1H), 7.21 (s, 1H), 6.69-6.60 (m, 3H), 6.04 (s, 1H), 2.75 (s, 3H), 1.50 (s, 3H); Chiral analytical SFC: RT=4.73 min, Column: Chiralpak IC-3, (4.6×150 mm, 3 μm), 60% CO₂/Methanol, Flow=3.0 g/min.

N-(1-(7,8-Difluoro-1-oxo-1,2-dihydroisoquinolin-4-yl)ethyl)-8-fluoro-N-methylindolizine-2-carboxamide—Enantiomer II (Compound 82), LCMS: m/z found 400.2 [M+H]⁺, RT=3.52 min (Method A); ¹H NMR (400 MHz, DMSO-d₆): δ 11.54 (bs, 1H), 8.11 (bs, 1H), 7.99 (s, 1H), 7.88-7.82 (m, 1H), 7.41 (s, 1H), 7.21 (s, 1H), 6.69-6.60 (m, 3H), 6.04 (s, 1H), 2.75 (s, 3H), 1.50 (s, 3H); Chiral analytical SFC: RT=6.52 min, Column: Chiralpak IC-3, (4.6×150 mm, 3 μm), 60% CO₂/Methanol, Flow=3.0 g/min.

### N-(1-(7,8-Difluoro-1-oxo-1, 2-dihydroisoquinolin-4-yl)ethyl)-5,5-difluoro-N-methyl-4,5,6,7-tetrahydro-1H-indole-2-carboxamide (Compounds 90 & 91)

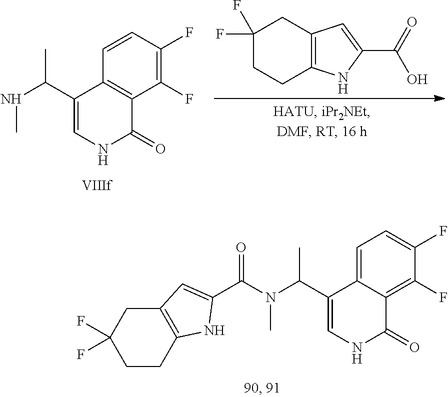

Racemic N-(1-(7,8-difluoro-1-oxo-1, 2-dihydroisoquinolin-4-yl) ethyl)-5,5-difluoro-N-methyl-4,5,6,7-tetrahydro-1H-indole-2-carboxamide was synthesized in a similar manner as described above from 7,8-difluoro-4-(1-(methylamino)ethyl)isoquinolin-1(2H)-one (VIIIf) and 5,5-difluoro-4,5,6,7-tetrahydro-1H-indole-2-carboxylic acid. The enantiomers were subsequently separated by chiral SFC, Column: Chiralpak AS-H (250×30 mm, 5 μm), 60% CO₂/Methanol, Flow rate 110 g/min.

N-(1-(7,8-Difluoro-1-oxo-1, 2-dihydroisoquinolin-4-yl)ethyl)-5,5-difluoro-N-methyl-4,5,6,7-tetrahydro-1H-indole-2-carboxamide—Enantiomer I (Compound 90), LCMS: m/z found 422.2 [M+H]⁺, RT=3.61 min (Method A); ¹H NMR (400 MHz, DMSO-d₆): δ 11.54 (bs, 1H), 11.32 (s, 1H), 7.80-7.73 (m, 1H), 7.35 (m, 1H), 7.20 (s, 1H), 6.29 (s, 1H), 6.01-5.99 (d, 1H), 2.96 (t, 2H), 2.80-2.73 (m, 5H), 2.26-2.15 (m, 2H), 1.45 (d, 3H); Chiral analytical SFC: RT=2.43 min, Column: Chiralpak AS-3, (4.6×150 mm, 3 m), 60% CO₂/Methanol, Flow=3.0 g/min.

N-(1-(7,8-Difluoro-1-oxo-1, 2-dihydroisoquinolin-4-yl)ethyl)-5,5-difluoro-N-methyl-4,5,6,7-tetrahydro-1H-indole-2-carboxamide—Enantiomer II (Compound 91), LCMS: m/z found 422.2 [M+H]⁺, RT=3.61 min (Method A); ¹H NMR (400 MHz, DMSO-d₆): δ 11.54 (bs, 1H), 11.32 (s, 1H), 7.80-7.73 (m, 1H), 7.35 (m, 1H), 7.20 (s, 1H), 6.29 (s, 1H), 6.01-5.99 (d, 1H), 2.96 (t, 2H), 2.80-2.73 (m, 5H), 2.26-2.15 (m, 2H), 1.45 (d, 3H); Chiral analytical SFC: RT=5.15 min, Column: Chiralpak AS-3, (4.6×150 mm, 3 m), 60% CO₂/Methanol, Flow=3.0 g/min.

### 8-Chloro-N-(1-(7,8-difluoro-1-oxo-1,2-dihydroisoquinolin-4-yl)ethyl)-N-methylindolizine-2-carboxamide (Compounds 92 & 93)

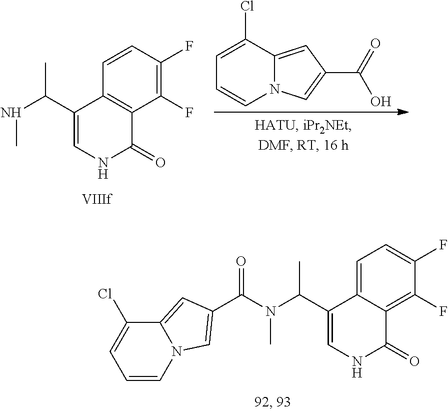

Racemic 8-chloro-N-(1-(7,8-difluoro-1-oxo-1,2-dihydroisoquinolin-4-yl)ethyl)-N-methylindolizine-2-carboxamide was synthesized in a similar manner as described above, from 7,8-difluoro-4-(1-(methylamino)ethyl)isoquinolin-1(2H)-one (VIIIf) and 8-chloroindolizine-2-carboxylic acid. The enantiomers were subsequently separated by chiral SFC, Column: Chiralpak IC (250×30 mm, 5 μm), 55% CO₂/Methanol, Flow rate 110 g/min.

8-Chloro-N-(1-(7,8-difluoro-1-oxo-1,2-dihydroisoquinolin-4-yl)ethyl)-N-methylindolizine-2-carboxamide—Enantiomer I (Compound 92), LCMS: m/z found 416.2/418.2 [M+H]⁺, RT=3.83 min (Method A); ¹H NMR (400 MHz, DMSO-d₆): δ 11.5 (bs, 1H), 8.24 (d, 1H), 7.98 (s, 1H), 7.85 (d, 1H), 7.43 (s, 1H), 7.20 (d, 1H), 6.94 (d, 1H), 6.66-6.59 (m, 2H), 6.05 (s, 1H), 2.75 (s, 3H), 1.50 (s, 3H); Chiral analytical SFC: RT=6.42 min, Column: Chiralpak IC-3, (4.6×150 mm, 3 μm), 60% CO₂/Methanol, Flow=3.0 g/min.

8-Chloro-N-(1-(7,8-difluoro-1-oxo-1,2-dihydroisoquinolin-4-yl)ethyl)-N-methylindolizine-2-carboxamide—Enantiomer II (Compound 93), LCMS: m/z found 416.2/418.2 [M+H]⁺, RT=3.83 min (Method A); ¹H NMR (400 MHz, DMSO-d₆): δ 11.5 (bs, 1H), 8.24 (d, 1H), 7.98 (s, 1H), 7.85 (d, 1H), 7.43 (s, 1H), 7.20 (d, 1H), 6.94 (d, 1H), 6.66-6.59 (m, 2H), 6.05 (s, 1H), 2.75 (s, 3H), 1.50 (s, 3H); Chiral analytical SFC: RT=9.20 min, Column: Chiralpak IC-3, (4.6×150 mm, 3 μm), 60% CO₂/Methanol, Flow=3.0 g/min.

### N-(1-(7,8-Difluoro-1-oxo-1,2-dihydroisoquinolin-4-yl)ethyl)-4,6-difluoro-N-methylindoline-2-carboxamide (Compounds 65, 66, 67, & 68)

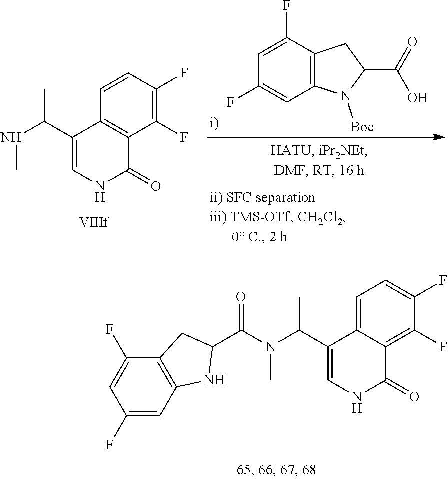 [SMILES: CNC(C)c1c[nH]c(=O)c2c(F)c(F)ccc12 ; CC(C)(C)OC(=O)N1c2cc(F)cc(F)c2CC1C(=O)O ; CC(c1c[nH]c(=O)c2c(F)c(F)ccc12)N(C)C(=O)C1Cc2c(F)cc(F)cc2N1]

tert-Butyl 2-((1-(7,8-difluoro-1-oxo-1,2-dihydroisoquinolin-4-yl)ethyl)(methyl)carbamoyl)-4,6-difluoroindoline-1-carboxylate (mixture of four stereoisomers) was synthesized in a similar manner as described above for Compounds 61-64 from 7,8-difluoro-4-(1-(methylamino)ethyl)isoquinolin-1(2H)-one (VIIIf) and racemic 1-(tert-butoxycarbonyl)-4,6-difluoroindoline-2-carboxylic acid. The stereoisomers were subsequently separated by chiral SFC, Column: Chiralcel OD (250×30 mm, 5 μm), 85% CO₂/Methanol, Flow rate 100 g/min to afford pure first (RT=4.16 min) and second (RT=5.23 min) eluting stereoisomers. The remaining two stereoisomers (overlapping RT=6.80 min) were separated by a second chiral SFC method: Column: DCPAK P4VP (250×30 mm, 5 μm), 80% CO₂/Methanol, Flow rate 100 g/min. Each isolated stereoisomer of tert-butyl 2-((1-(7,8-difluoro-1-oxo-1,2-dihydroisoquinolin-4-yl)ethyl)(methyl)carbamoyl)-4,6-difluoroindoline-1-carboxylate was converted to a single stereoisomer of N-(1-(7,8-difluoro-1-oxo-1,2-dihydroisoquinolin-4-yl)ethyl)-4,6-difluoro-N-methylindoline-2-carboxamide by treatment with TMSOTf in a similar manner as described above.

N-(1-(7,8-Difluoro-1-oxo-1,2-dihydroisoquinolin-4-yl)ethyl)-4,6-difluoro-N-methylindoline-2-carboxamide—Stereoisomer IIA (Compound 65) LCMS: m/z found 420.2 [M+H]⁺, RT=3.73 min (Method A); ¹H NMR (400 MHz, DMSO-d₆): δ 11.54 (bs, 1H), 7.70-7.63 (m, 1H), 7.17-7.12 (m, 2H), 6.59-6.48 (m, 1H), 6.26-6.20 (m, 2H), 5.88-5.82 (m, 1H), 4.80-4.76 (m, 1H), 3.23-3.16 (m, 1H), 2.70-2.65 (m, 1H), 2.58 (s, 3H), 1.39 (d, 3H); Chiral analytical SFC: RT=3.74 min, Column: Chiralpak AS-H, (4.6×150 mm, 3 μm), 60% CO₂/(0.2% DEA in Methanol), Flow=3.0 g/min.

N-(1-(7,8-Difluoro-1-oxo-1,2-dihydroisoquinolin-4-yl)ethyl)-4,6-difluoro-N-methylindoline-2-carboxamide—Stereoisomer IA (Compound 66, enantiomer of Compound 65), LCMS: m/z found 420.2 [M+H]⁺, RT=3.73 min (Method A); ¹H NMR (400 MHz, DMSO-d₆): δ 11.54 (bs, 1H), 7.70-7.63 (m, 1H), 7.17-7.12 (m, 2H), 6.59-6.48 (m, 1H), 6.26-6.20 (m, 2H), 5.88-5.82 (m, 1H), 4.80-4.76 (m, 1H), 3.23-3.16 (m, 1H), 2.70-2.65 (m, 1H), 2.58 (s, 3H), 1.39 (d, 3H); Chiral analytical SFC: RT=1.94 min, Column: Chiralpak AS-H, (4.6×150 mm, 3 μm), 60% CO₂/(0.2% DEA in Methanol), Flow=3.0 g/min.
N-(1-(7,8-Difluoro-1-oxo-1,2-dihydroisoquinolin-4-yl)ethyl)-4,6-difluoro-N-methylindoline-2-carboxamide—Stereoisomer IIB (Compound 67), LCMS: m/z found 420.2 [M+H]⁺, RT=3.76 min (Method A); ¹H NMR (400 MHz, DMSO-d₆): δ 11.54 (bs, 1H), 7.76-7.69 (m, 1H), 7.36-7.33 (m, 1H), 7.17 (d, 1H), 6.46 (1H, s), 6.25-6.15 (m, 2H), 5.92-5.87 (m, 1H), 4.77-4.72 (m, 1H), 3.34-3.28 (m, 1H), 3.06-3.01 (m, 1H), 2.62 (s, 3H), 1.37 (d, 3H); Chiral analytical SFC: RT=2.91 min, Column: Chiralpak AS-H, (4.6×150 mm, 3 μm), 60% CO₂/(0.2% DEA in Methanol), Flow=3.0 g/min.

N-(1-(7,8-Difluoro-1-oxo-1,2-dihydroisoquinolin-4-yl)ethyl)-4,6-difluoro-N-methylindoline-2-carboxamide—Stereoisomer IB (Compound 68, enantiomer of Compound 67), LCMS: m/z found 420.2 [M+H]⁺, RT=3.76 min (Method A); ¹H NMR (400 MHz, DMSO-d₆): δ 11.54 (br s, 1H), 7.76-7.69 (m, 1H), 7.36-7.33 (m, 1H), 7.17 (d, 1H), 6.46 (1H, s), 6.25-6.15 (m, 2H), 5.92-5.87 (m, 1H), 4.77-4.72 (m, 1H), 3.34-3.28 (m, 1H), 3.06-3.01 (m, 1H), 2.62 (s, 3H), 1.37 (d, 3H); Chiral analytical SFC: RT=1.71 min, Column: Chiralpak AS-H, (4.6×150 mm, 3 m), 60% CO₂/(0.2% DEA in Methanol), Flow=3.0 g/min.

### N-(1-(7,8-Difluoro-1-oxo-1,2-dihydroisoquinolin-4-yl)ethyl)-5-fluoro-N-methylindoline-2-carboxamide (Compounds 73, 74, 75, & 76)

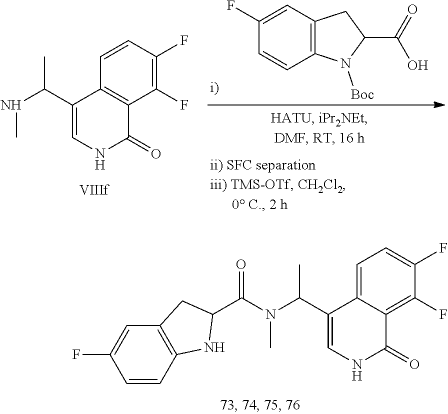 [SMILES: CNC(C)c1c[nH]c(=O)c2c(F)c(F)ccc12 ; CC(C)(C)OC(=O)N1c2ccc(F)cc2CC1C(=O)O ; CC(c1c[nH]c(=O)c2c(F)c(F)ccc12)N(C)C(=O)C1Cc2cc(F)ccc2N1]

tert-Butyl2-((1-(7,8-difluoro-1-oxo-1,2-dihydroisoquinolin-4-yl)ethyl)(methyl)carbamoyl)-5-fluoroindoline-1-carboxylate (mixture of four stereoisomers) was synthesized in a similar manner as described above from 7,8-difluoro-4-(1-(methylamino)ethyl)isoquinolin-1(2H)-one (VIIIf) and racemic 1-(tert-butoxycarbonyl)-5-fluoroindoline-2-carboxylic acid. The stereoisomers were subsequently separated by chiral SFC, Column: Chiralpak IG (250×30 mm, 5 m), 75% CO₂/Methanol, Flow rate 110 g/min to afford pure last two eluting stereoisomers. The remaining two stereoisomers were separated by a second chiral SFC method: Column: Chiralcel OD (250×30 mm, 5 μm), 80% CO₂/Methanol, Flow rate 110 g/min. Each isolated stereoisomer of tert-butyl 2-((1-(7,8-difluoro-1-oxo-1,2-dihydroisoquinolin-4-yl)ethyl)(methyl)carbamoyl)-4,6-difluoroindoline-1-carboxylate was converted to a single stereoisomer of N-(1-(7,8-difluoro-1-oxo-1,2-dihydroisoquinolin-4-yl)ethyl)-5-fluoro-N-methylindoline-2-carboxamide by treatment with TMSOTf in a similar manner as described above.

N-(1-(7,8-Difluoro-1-oxo-1,2-dihydroisoquinolin-4-yl)ethyl)-5-fluoro-N-methylindoline-2-carboxamide—Stereoisomer IIA (Compound 73), LCMS: m/z found 402.2 [M+H]⁺, RT=2.59 min (Method A); ¹H NMR (400 MHz, DMSO-d₆): δ 11.53 (bs, 1H), 7.78-7.71 (m, 1H), 7.37-7.34 (m, 1H), 7.16 (s, 1H), 6.88-6.85 (m, 1H), 6.77-6.72 (m, 1H), 6.33-6.49 (m, 1H), 5.93-5.88 (m, 1H), 5.57 (d, 1H), 4.62-4.58 (m, 1H), 3.32-3.28 (m, 1H), 3.13-3.07 (m, 1H), 2.62 (s, 3H), 1.36 (d, 3H); Chiral analytical SFC: RT=3.50 min, Column: Chiralpak AS-H, (4.6×150 mm, 5 μm), 60% CO₂/Methanol, Flow=3.0 g/min.

N-(1-(7,8-Difluoro-1-oxo-1,2-dihydroisoquinolin-4-yl)ethyl)-5-fluoro-N-methylindoline-2-carboxamide—Stereoisomer IA (Compound 74, enantiomer of Compound 73), LCMS: m/z found 402.2 [M+H]⁺, RT=2.59 min (Method A); ¹H NMR (400 MHz, DMSO-d₆): δ 11.53 (bs, 1H), 7.78-7.71 (m, 1H), 7.37-7.34 (m, 1H), 7.16 (s, 1H), 6.88-6.85 (m, 1H), 6.77-6.72 (m, 1H), 6.33-6.49 (m, 1H), 5.93-5.88 (m, 1H), 5.57 (d, 1H), 4.62-4.58 (m, 1H), 3.32-3.28 (m, 1H), 3.13-3.07 (m, 1H), 2.62 (s, 3H), 1.36 (d, 3H); Chiral analytical SFC: RT=1.85 min, Column: Chiralpak AS-H, (4.6×150 mm, 5 μm), 60% CO₂/Methanol, Flow=3.0 g/min.

N-(1-(7,8-Difluoro-1-oxo-1,2-dihydroisoquinolin-4-yl)ethyl)-5-fluoro-N-methylindoline-2-carboxamide—Stereoisomer IIB (Compound 75), LCMS: m/z found 402.3 [M+H]⁺, RT=2.69 min (Method A); ¹H NMR (400 MHz, DMSO-d₆): δ 11.54 (bs, 1H), 7.74-7.67 (m, 1H), 7.16-7.13 (m, 2H), 6.84-6.82 (d, 1H), 6.78-6.73 (m, 1H), 6.56-6.52 (m, 1H), 5.89-5.84 (m, 1H), 5.63 (s, 1H), 4.66-4.63 (m, 1H), 3.33-3.18 (m, 1H), 2.82-2.76 (m, 1H), 2.58 (s, 3H), 1.38 (d, 3H); Chiral analytical SFC: RT=3.40 min, Column: Chiralpak IC-3, (4.6×150 mm, 3 μm), 60% CO₂/(0.2% DEA in Methanol), Flow=3.0 g/min.

N-(1-(7,8-Difluoro-1-oxo-1,2-dihydroisoquinolin-4-yl)ethyl)-5-fluoro-N-methylindoline-2-carboxamide—Stereoisomer IB (Compound 76, enantiomer of Compound 75), LCMS: m/z found 402.2 [M+H]⁺, RT=2.69 min (Method A); ¹H NMR (400 MHz, DMSO-d₆): δ 11.54 (bs, 1H), 7.74-7.67 (m, 1H), 7.16-7.13 (m, 2H), 6.84-6.82 (d, 1H), 6.78-6.73 (m, 1H), 6.56-6.52 (m, 1H), 5.89-5.84 (m, 1H), 5.63 (s, 1H), 4.66-4.63 (m, 1H), 3.33-3.18 (m, 1H), 2.82-2.76 (m, 1H), 2.58 (s, 3H), 1.38 (d, 3H); Chiral analytical SFC: RT=2.93 min, Column: Chiralpak IC-3, (4.6×150 mm, 3 μm), 60% CO₂/(0.2% DEA in Methanol), Flow=3.0 g/min.

### N-(1-(7,8-Difluoro-1-oxo-1,2-dihydroisoquinolin-4-yl)ethyl)-N,3,3-trimethylindoline-2-carboxamide (Compounds 104, 105, 106, & 107)

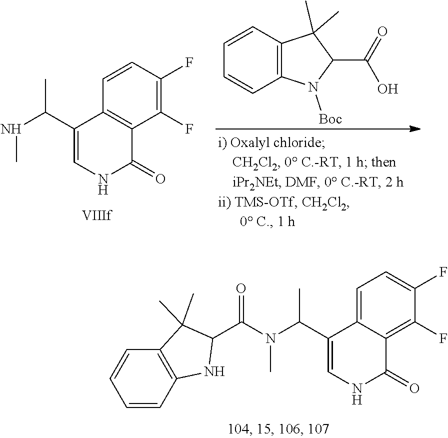

N-(1-(7,8-Difluoro-1-oxo-1,2-dihydroisoquinolin-4-yl)ethyl)-N,3,3-trimethylindoline-2-carboxamide (mixture of four stereoisomers) was synthesized in a similar manner as described above for Compounds 112-115 from 7,8-difluoro-4-(1-(methylamino)ethyl)isoquinolin-1(2H)-one (VIIIf) and racemic 1-(tert-butoxycarbonyl)-3,3-dimethylindoline-2-carboxylic acid. The mixture of stereoisomers was fractionated by chiral SFC, Column: DCPAK P4CP (250×21 mm, 5 μm) 75% CO₂/Methanol, Flow rate 70 g/min into two diastereomeric racemates. The enantiomers were subsequently separated by chiral SFC, under the same conditions for each of the racemates: Column: Chiralpak-AS-H (250×30 mm, 5 μm) 60% CO₂/Methanol, Flow rate 100 g/min.

N-(1-(7,8-Difluoro-1-oxo-1,2-dihydroisoquinolin-4-yl)ethyl)-N,3,3-trimethylindoline-2-carboxamide—Stereoisomer IA (Compound 104), LCMS: m/z found 412.2 [M+H]⁺, RT=2.94 min (Method A); ¹H NMR (400 MHz, DMSO-d₆): δ 11.53 (bs, 1H), 7.79-7.71 (m, 1H), 7.37-7.33 (m, 1H), 7.15 (s, 1H), 6.96-6.88 (m, 2H), 6.64-6.60 (m, 2H), 5.95-5.90 (m, 1H), 5.67 (s, 1H), 4.31 (s, 1H), 2.62 (s, 3H), 1.36 (d, 3H), 1.32 (s, 3H), 1.22 (s, 3H); Chiral analytical SFC: RT=1.80 min, Column: Chiralcel OX-3, (4.6×150 mm, 3 μm), 60% CO₂/Ethanol, Flow=3.0 g/min.

N-(1-(7,8-Difluoro-1-oxo-1,2-dihydroisoquinolin-4-yl)ethyl)-N,3,3-trimethylindoline-2-carboxamide—Stereoisomer IIA (Compound 105, enantiomer of Compound 104), LCMS: m/z found 412.2 [M+H]⁺, RT=2.94 min (Method A); ¹H NMR (400 MHz, DMSO-d₆): δ 11.53 (bs, 1H), 7.79-7.71 (m, 1H), 7.37-7.33 (m, 1H), 7.15 (s, 1H), 6.96-6.88 (m, 2H), 6.64-6.60 (m, 2H), 5.95-5.90 (m, 1H), 5.67 (s, 1H), 4.31 (s, 1H), 2.62 (s, 3H), 1.36 (d, 3H), 1.32 (s, 3H), 1.22 (s, 3H); Chiral analytical SFC: RT=2.42 min, Column: Chiralcel OX-3, (4.6×150 mm, 3 μm), 60% CO₂/Ethanol, Flow=3.0 g/min.

N-(1-(7,8-Difluoro-1-oxo-1,2-dihydroisoquinolin-4-yl)ethyl)-N,3,3-trimethylindoline-2-carboxamide—Stereoisomer IB (Compound 106), LCMS: m/z found 412.3 [M+H]⁺, RT=3.04 min (Method A); ¹H NMR (400 MHz, DMSO-d₆): δ 11.54 (bs, 1H), 7.82-7.75 (m, 1H), 7.44-7.41 (m, 1H), 7.21 (s, 1H), 6.94-6.88 (m, 2H), 6.56-6.51 (m, 2H), 6.03-5.98 (m, 1H), 5.68 (s, 1H), 4.37 (s, 1H), 2.69 (s, 3H), 1.40 (d, 3H), 1.23 (s, 3H), 0.99 (s, 3H); Chiral analytical SFC: RT=1.14 min, Column: Chiralpak AS-3, (4.6×150 mm, 3 μm), 60% CO₂/(0.2% 7M Methanolic Ammonia in 1:1 v/v Acetonitrile-Methanol), Flow=3.0 g/min.

N-(1-(7,8-Difluoro-1-oxo-1,2-dihydroisoquinolin-4-yl)ethyl)-N,3,3-trimethylindoline-2-carboxamide—Stereoisomer IIB (Compound 107, enantiomer of Compound 106), LCMS: m/z found 412.3 [M+H]⁺, RT=3.04 min (Method A); ¹H NMR (400 MHz, DMSO-d₆): δ 11.54 (bs, 1H), 7.82-7.75 (m, 1H), 7.44-7.41 (m, 1H), 7.21 (s, 1H), 6.94-6.88 (m, 2H), 6.56-6.51 (m, 2H), 6.03-5.98 (m, 1H), 5.68 (s, 1H), 4.37 (s, 1H), 2.69 (s, 3H), 1.40 (d, 3H), 1.23 (s, 3H), 0.99 (s, 3H); Chiral analytical SFC: RT=3.29 min, Column: Chiralpak AS-3, (4.6×150 mm, 3 μm), 60% CO₂/(0.2% 7M Methanolic Ammonia in 1:1 v/v Acetonitrile-Methanol), Flow=3.0 g/min.

### N-(1-(7,8-Difluoro-1-oxo-1,2-dihydroisoquinolin-4-yl)ethyl)-3-fluoro-N-isobutyl-4-(trifluoromethyl)benzamide (Compound 118)

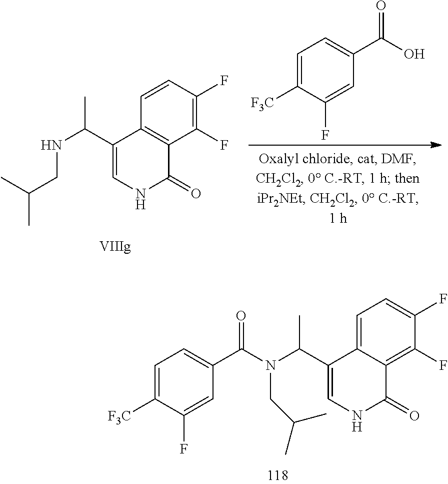 [SMILES: CC(C)CNC(C)c1c[nH]c(=O)c2c(F)c(F)ccc12 ; O=C(O)c1ccc(C(F)(F)F)c(F)c1 ; CC(C)CN(C(=O)c1ccc(C(F)(F)F)c(F)c1)C(C)c1c[nH]c(=O)c2c(F)c(F)ccc12]

Racemic N-(1-(7,8-Difluoro-1-oxo-1,2-dihydroisoquinolin-4-yl)ethyl)-3-fluoro-N-isobutyl-4-(trifluoromethyl)benzamide was synthesized in a similar manner as described above for Compound 117, from 7,8-difluoro-4-(1-(isobutylamino)ethyl)isoquinolin-1(2H)-one (VIIIg, derived from ketone XXb in a similar manner as described above) and 3-fluoro-4-(trifluoromethyl)benzoic acid. LCMS: m/z found 471.4 [M+H]⁺, RT=1.89 min (Method D); ¹H NMR (400 MHz, DMSO-d₆): δ 11.57 (bs, 1H), 8.01-7.94 (m, 1H), 7.85 (t, 1H), 7.55 (d, 1H), 7.47 (m, 1H), 7.33 (d, 2H), 6.00 (m, 1H), 2.85-2.66 (m, 2H), 1.60 (d, 3H), 1.33-1.15 (m, 1H), 0.40 (dd, 6H).

### (R)-7,8-Difluoro-4-(1-(methylamino)ethyl)isoquinolin-1(2H)-one hydrochloride salt ((R)-VIIIf)

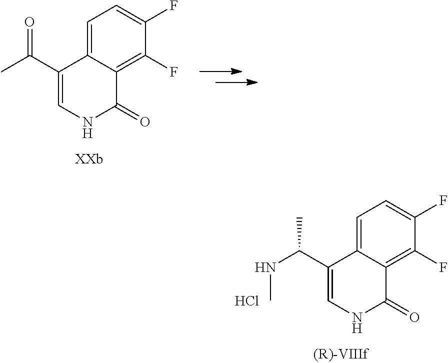 [SMILES: CC(=O)c1c[nH]c(=O)c2c(F)c(F)ccc12 ; Cl ; CN[C@H](C)c1c[nH]c(=O)c2c(F)c(F)ccc12]

(R)-7,8-Difluoro-4-(1-(methylamino)ethyl)isoquinolin-1(2H)-one hydrochloride salt ((R)-VIIIf) was synthesized from 4-acetyl-7,8-difluoroisoquinolin-1(2H)-one (XXb) in a similar manner as described above for (R)-6,7-difluoro-4-(1-(methylamino)ethyl)isoquinolin-1(2H)-one hydrochloride salt ((R)-VIIIa).

The absolute configuration of the α-methyl substituent of (R)-XIIIf has been inferred from previous X-ray crystallographic studies, wherein 1-(1-methoxy-4-isoquinolyl)ethanone was used as the ketone substrate in place of XXb, under identical conditions as those described herein, as detailed in WO 2020123674.

### (R)-N-(1-(7,8-Difluoro-1-oxo-1,2-dihydroisoquinolin-4-yl)ethyl)-N-methyl-6-oxo-1,6-dihydropyridine-2-carboxamide (Compound 87)

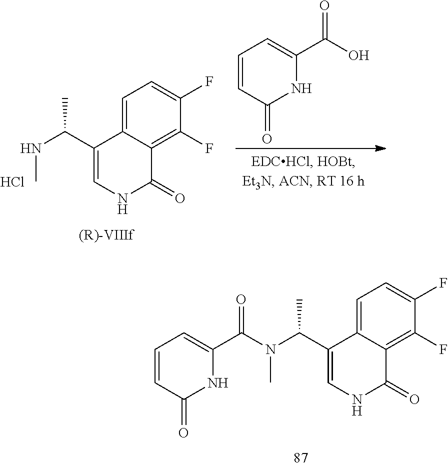

N-Ethyl-N′-(3-dimethylaminopropyl)carbodiimide hydrochloride (24.4 mg, 0.13 mmol) and triethylamine (24 μL, 0.17 mmol) were added to a mixture of (R)-7,8-difluoro-4-(1-(methylamino)ethyl)isoquinolin-1(2H)-one hydrochloride salt ((R)-VIIIf, 31.8 mg, 0.12 mmol), HOBt hydrate (17.7 mg, 0.12 mmol), and 6-oxo-1,6-dihydropyridine-2-carboxylic acid (16 mg, 0.12 mmol) in 1 mL of acetonitrile and the reaction mixture was allowed to stir at room temperature for 16 h. The reaction mixture was concentrated in under reduced pressure and the residue was suspended in 1 mL of 1:1 v/v water/acetonitrile and 2.5 mL DMSO, and the suspension was filtered through a syringe filter. The material was purified by reverse phase HPLC (C18 column, 5-65% acetonitrile/water gradient, 0.01% TFA as a modifier) to provide (R)-N-(1-(7,8-difluoro-1-oxo-1,2-dihydroisoquinolin-4-yl)ethyl)-N-methyl-6-oxo-1,6-dihydropyridine-2-carboxamide (18.3 mg, 44% yield). LCMS: m/z found 360.2 [M+H]f, RT=1.84 min (Method A); ¹H NMR (400 MHz, CD3OD): δ 7.74 (td, 1H), 7.59 (t, 1H), 7.51 (dd, 1H), 7.29 (s, 1H), 6.62 (d, 1H), 6.42 (s, 1H), 6.13 (d, 1H), 2.66 (s, 3H), 1.62 (d, 3H).

### (S,E)-N-((1-Methoxyisoquinolin-4-yl)methylene)-2-methylpropane-2-sulfinamide (XIIb)

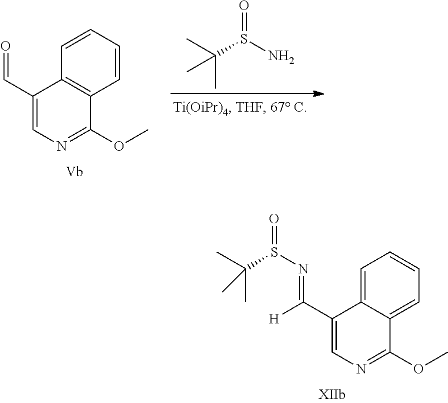

To a solution of 5.0 g (26.7 mmol, 1.0 eq.) of 1-methoxyisoquinoline-4-carbaldehyde (Vb) in 90 mL of anhydrous THE at room temperature under a nitrogen atmosphere was added 15.8 mL (53.4 mmol, 2.0 eq.) of titanium (IV) isopropoxide followed by 3.56 g (29.4 mmol, 1.1 eq.) of (S)-2-methylpropane-2-sulfinamide, and the mixture was heated at 67° C. for 16 h. The mixture was allowed to cool to room temperature and poured into 100 mL of a rapidly stirred brine solution. The mixture was stirred for 10 minutes and then filtered through CELITE®. The filter cake was washed with 500 mL of ethyl acetate, and the filtrate was transferred to a separating funnel where the layers were separated. The organic phase was washed with brine (100 mL) and the combined aqueous washings were extracted with ethyl acetate (100 ml). The combined organic extracts were dried (over Na₂SO₄), filtered, and evaporated, and dried under high vacuum to provide 7.3 g of crude (S,E)—N-((1-methoxyisoquinolin-4-yl)methylene)-2-methylpropane-2-sulfinamide (XIIb), which was used without further purification. LCMS m/z found 291.13 [M+H]⁺, RT=2.16 min (Method E), ¹H NMR (400 MHz, DMSO-d₆): δ 9.18 (d, 1H), 8.78 (s, 1H), 8.65 (s, 1H), 8.33-8.31 (dd, 1H), 7.96 (t, 1H), 7.76 (t, 1H), 4.16 (s, 3H), 1.24 (s, 9H).

### (S)-N-((R)-1-(1-Methoxyisoquinolin-4-yl)ethyl)-2-methylpropane-2-sulfinamide (XIIIb)

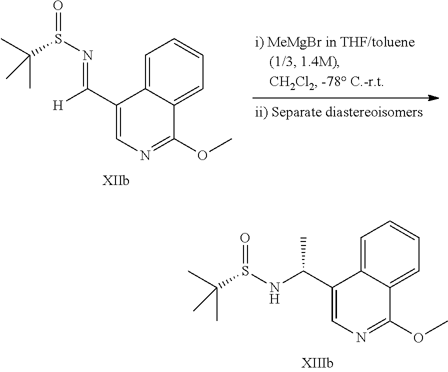 [SMILES: COc1ncc(/C=N/[S@@](=O)C(C)(C)C)c2ccccc12 ; COc1ncc([C@@H](C)N[S@@](=O)C(C)(C)C)c2ccccc12]

A solution of 7.3 g of crude (S,E)-N-((1-methoxyisoquinolin-4-yl)methylene)-2-methylpropane-2-sulfinamide (XIIb) in 150 mL of anhydrous methylene chloride under a nitrogen atmosphere was cooled to −78° C. and 28.6 mL (62.9 mmol) of a 1.4 M solution of methylmagnesium bromide in a THF:toluene mixture, 1:3 (v/v) was added slowly. The mixture was allowed to warm to room temperature and stirred for 16 h. The reaction mixture was then slowly added to a mixture of 80 mL of saturated aqueous ammonium chloride and ice. The resulting mixture was diluted with 200 mL of ethyl acetate and the layers were separated. The aqueous phase was extracted with 2×1000 mL of ethyl acetate and the combined organic extracts were washed with 50 mL of saturated sodium bicarbonate solution, dried (Na₂SO₄), filtered, and the solvent was evaporated in vacuo. The residue was purified by flash chromatography (SiO₂, eluting with 40%-100% ethyl acetate/hexanes) to obtain 3.5 g (11.42 mmol, 45% from Vb) of the major diastereomer (S)-N-((R)-1-(1-methoxyisoquinolin-4-yl)ethyl)-2-methylpropane-2-sulfinamide. LCMS m/z found 307.36 [M+H]^{m}, RT=1.80 min (Method E), ¹H NMR (400 MHz, DMSO-d₆): δ 8.21 (d, 1H), 8.15 (d, 1H), 8.04 (s, 1H), 7.78 (t, 1H), 7.62 (t, 1H), 5.44 (d, 1H), 4.95 (t, 1H), 4.05 (s, 3H), 1.65 (d, 3H), 1.07 (s, 9H).

The absolute configuration of the α-methyl substituent of XIIIb has been confirmed by comparative analysis in view of previous X-ray crystallographic studies, as detailed in WO 2020123674.

### (S)-N-((R)-1-(1-Methoxyisoquinolin-4-yl)ethyl)-N,2-dimethylpropane-2-sulfinamide (XIVb)

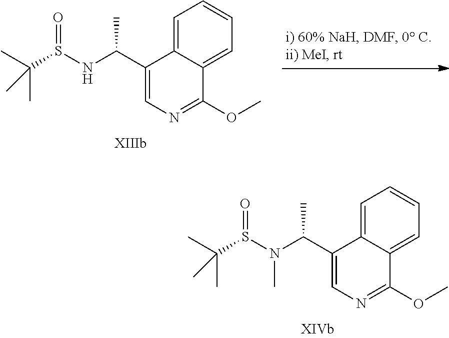 [SMILES: COc1ncc([C@@H](C)N[S@@](=O)C(C)(C)C)c2ccccc12 ; COc1ncc([C@@H](C)N(C)[S@@](=O)C(C)(C)C)c2ccccc12]

To a solution of 2.2 g (7.2 mmol, 1.0 eq.) of (S)-N-((R)-1-(1-methoxyisoquinolin-4-yl)ethyl)-2-methylpropane-2-sulfinamide (XIIIb) in 66 mL of anhydrous DMF under a nitrogen atmosphere at 0° C. was added 0.57 g (14.4 mmol, 2.0 eq.) of a 60% dispersion of sodium hydride in mineral oil. The mixture was stirred at 0° C. for 20 min and 0.89 mL (14.4 mmol, 2 eq.) of iodomethane was added. The mixture was stirred at 0° C. for an additional 2 h and quenched by the slow addition of 100 mL of water. The mixture was extracted with 3×50 mL of ethyl acetate. The combined organic extracts were washed with 3×25 mL of water, 25 mL of brine, dried (Na₂SO₄), filtered, and the solvent was evaporated in vacuo. The residue was purified by flash chromatography (SiO₂, eluting with 25-100% ethyl acetate/hexanes) to obtain 1.8 g (5.62 mmol, 78% yield) of (S)-N-((R)-1-(1-methoxyisoquinolin-4-yl)ethyl)-N,2-dimethylpropane-2-sulfinamide (XIVb). LCMS m/z found 321.48 [M+H]+, RT=1.96 min (Method E), ¹H NMR (400 MHz, CDCl₃): δ 8.30 (d, 1H), 8.03-7.98 (m, 2H), 7.70 (m, 1H), 7.56 (m, 1H), 5.25-5.19 (m, 1H), 4.13 (s, 3H), 2.41 (s, 3H), 1.76 (d, 3H), 1.21 (s, 9H).

### (R)-4-(1-(Methylamino)ethyl)isoquinolin-1(2H)-one ((R)-VIIIh)

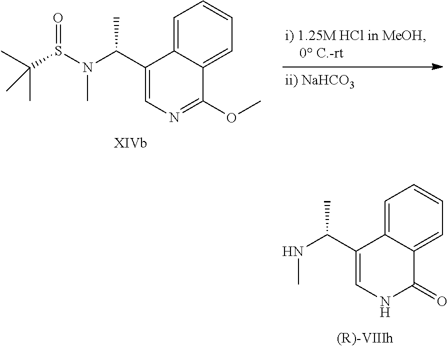 [SMILES: COc1ncc([C@@H](C)N(C)[S@@](=O)C(C)(C)C)c2ccccc12 ; CN[C@H](C)c1c[nH]c(=O)c2ccccc12]

A solution of 3 g (9.4 mmol, 1.0 eq.) of diastereomerically pure (S)-N-((R)-1-(1-methoxy isoquinolin-4-yl)ethyl)-N,2-dimethylpropane-2-sulfinamide (XIVb) in 113 mL (234.4 mmol, 25 eq.) of a 1.25 M solution of HCl in methanol in a sealed tube was stirred at room temperature for 16 h. The volatiles were evaporated in vacuo to provide a white solid which was suspended in 40 mL of 2-methyl THF and 80 mL of diethyl ether. The mixture was cooled in an ice bath and the resulting white precipitate was collected by vacuum filtration and dried under high vacuum to provide 1.78 g of (R)-4-(1-(methylamino)ethyl)isoquinolin-1(2H)-one hydrochloride. The obtained HCl salt was taken in EtOAc (60 mL) and basified using a saturated Na₂CO₃ solution. The organic layer was separated, dried (Na₂SO₄), and evaporated to dryness to obtain 1.1 g (5.44 mmol, 75% yield) of (R)-4-(1-(methylamino)ethyl)isoquinolin-1(2H)-one ((R)-VIIIh) as a single enantiomer. LCMS m/z found 203.22 [M+H]⁺, RT=0.67 min (Method E), ¹H NMR (400 MHz, DMSO-d₆) δ 11.62 (d, 1H), 9.64 (s, 1H), 9.13 (s, 1H), 8.26 (m, 1H), 7.94 (d, 1H), 7.78 (m, 1H), 7.51-7.61 (m, 2H), 4.80 (q, 1H), 2.52 (m, 3H, overlapping with DMSO-d₆), 1.58 (d, 3H).

### (R)-3-Fluoro-N-methyl-N-(1-(1-oxo-1,2-dihydroisoquinolin-4-yl)ethyl)-4-(trifluoromethyl)benzamide (Compound 145)

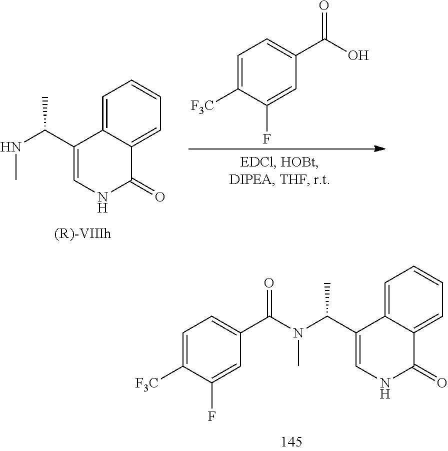 [SMILES: CN[C@H](C)c1c[nH]c(=O)c2ccccc12 ; O=C(O)c1ccc(C(F)(F)F)c(F)c1 ; C[C@H](c1c[nH]c(=O)c2ccccc12)N(C)C(=O)c1ccc(C(F)(F)F)c(F)c1]

Enantiomerically pure (R)-3-fluoro-N-methyl-N-(1-(1-oxo-1,2-dihydroisoquinolin-4-yl)ethyl)-4-(trifluoromethyl)benzamide was synthesized in a similar manner as described above (General procedure III), starting from (R)-4-(1-(methylamino)ethyl)isoquinolin-1(2H)-one ((R)-VIIIh) and 3-fluoro-4-(trifluoromethyl)benzoic acid. LCMS: m/z found 393.3 [M+H]⁺, RT=5.55 min (Method A); ¹H NMR (400 MHz, DMSO-d₆): δ 11.44 (s, 1H), 8.28 (d, 1H), 7.84-7.82 (m, 2H), 7.67 (d, 1H), 7.56-7.50 (m, 2H), 7.29 (d, 1H), 7.22 (d, 1H), 6.07 (q, 1H), 2.43 (s, 3H), 1.54 (d, 3H).

### (R)-4-Bromo-N-methyl-N-(1-(1-oxo-1,2-dihydroisoquinolin-4-yl)ethyl)benzamide (Compound 146)

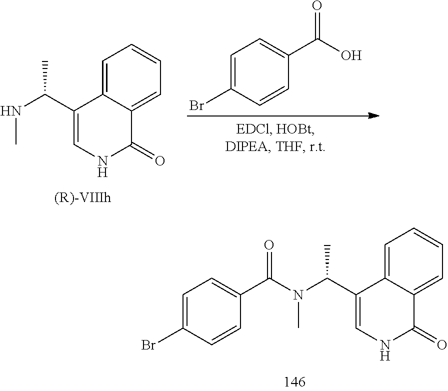

Enantiomerically pure (R)-4-bromo-N-methyl-N-(1-(1-oxo-1,2-dihydroisoquinolin-4-yl)ethyl)benzamide was synthesized in a similar manner as described above (General procedure III), starting from (R)-4-(1-(methylamino)ethyl)isoquinolin-1(2H)-one ((R)-VIIIh) and 4-bromobenzoic acid. LCMS: m/z found 387.2 [M+H]⁺, RT=5.22 min (Method A); ¹H NMR (400 MHz, DMSO-d₆): δ 11.41 (s, 1H), 8.27 (d, 1H), 7.79 (t, 1H), 7.65-7.61 (m, 3H), 7.55 (t, 1H), 7.24 (d, 2H), 7.20 (s, 1H), 6.05 (m, 1H), 2.42 (s, 3H), 1.52 (d, 3H).

### (R)-N-Methyl-N-(1-(1-oxo-1,2-dihydroisoquinolin-4-yl)ethyl)-4-(trifluoromethyl)benzamide (Compound 147)

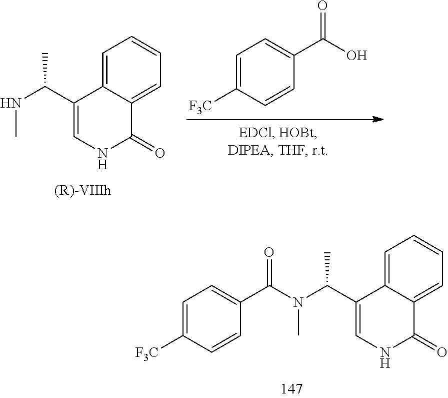 [SMILES: CN[C@H](C)c1c[nH]c(=O)c2ccccc12 ; O=C(O)c1ccc(C(F)(F)F)cc1 ; C[C@H](c1c[nH]c(=O)c2ccccc12)N(C)C(=O)c1ccc(C(F)(F)F)cc1]

Enantiomerically pure (R)-N-methyl-N-(1-(1-oxo-1,2-dihydroisoquinolin-4-yl)ethyl)-4-(trifluoromethyl)benzamide was synthesized in a similar manner as described above (General procedure III), starting from (R)-4-(1-(methylamino)ethyl)isoquinolin-1(2H)-one ((R)-VIIIh) and 4-(trifluoromethyl)benzoic acid. LCMS: m/z found 375.3 [M+H]⁺, RT=5.38 min (Method A); ¹H NMR (400 MHz, DMSO-d₆): δ 11.44 (s, 1H), 8.28 (d, 1H), 7.85-7.83 (m, 3H), 7.68 (d, 1H), 7.58-7.49 (m, 3H), 7.22 (d, 1H), 6.12-6.07 (m, 1H), 2.42 (s, 3H), 1.54 (d, 3H).

### (R)-4-Chloro-3-fluoro-N-methyl-N-(1-(1-oxo-1,2-dihydroisoquinolin-4-yl)ethyl)benzamide (Compound 148)

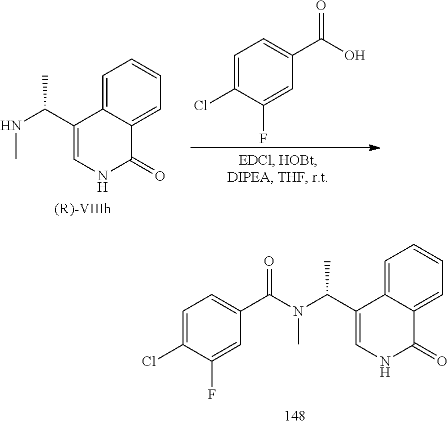

Enantiomerically pure (R)-4-chloro-3-fluoro-N-methyl-N-(1-(1-oxo-1,2-dihydroisoquinolin-4-yl)ethyl)benzamide was synthesized in a similar manner as described above (General procedure III), starting from (R)-4-(1-(methylamino)ethyl)isoquinolin-1(2H)-one ((R)-VIIIh) and 4-chloro-3-fluorobenzoic acid. LCMS: m/z found 359.2 [M+H]⁺, RT=7.87 min (Method A); ¹H NMR (400 MHz, DMSO-d₆): δ 11.43 (s, 1H), 8.27 (d, 1H), 7.80 (t, 1H), 7.65-7.62 (m, 2H), 7.55 (t, 1H), 7.40 (d, 1H), 7.21 (d, 1H), 7.13 (d, 1H), 6.05 (m, 1H), 2.44 (s, 3H), 1.53 (d, 3H).

### (R)-3-Chloro-4-fluoro-N-methyl-N-(1-(1-oxo-1,2-dihydroisoquinolin-4-yl)ethyl)benzamide (Compound 149)

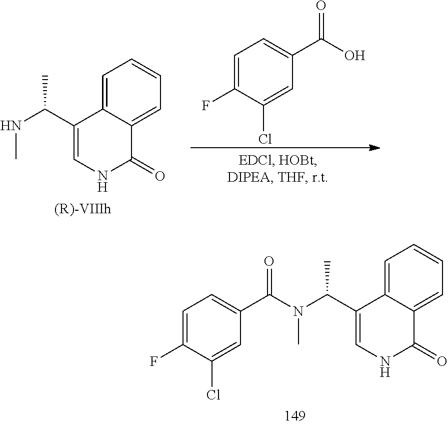

Enantiomerically pure (R)-3-chloro-4-fluoro-N-methyl-N-(1-(1-oxo-1,2-dihydroisoquinolin-4-yl)ethyl)benzamide was synthesized in a similar manner as described above (General procedure III), starting from (R)-4-(1-(methylamino)ethyl)isoquinolin-1(2H)-one ((R)-VIIIh) and 3-chloro-4-fluorobenzoic acid. LCMS: m/z found 359.2 [M+H]⁺, RT=7.77 min (Method A); ¹H NMR (400 MHz, DMSO-d₆): δ 11.38 (s, 1H), 8.27 (d, 1H), 7.80 (t, 1H), 7.64 (t, 1H), 7.57-7.53 (m, 2H), 7.44 (d, 1H), 7.30 (br s, 1H), 7.20 (d, 1H), 6.05 (q, 1H), 2.47 (s, 3H), 1.53 (d, 3H).

### (R)-4-Bromo-3-fluoro-N-methyl-N-(1-(1-oxo-1,2-dihydroisoquinolin-4-yl)ethyl)benzamide (Compound 150)

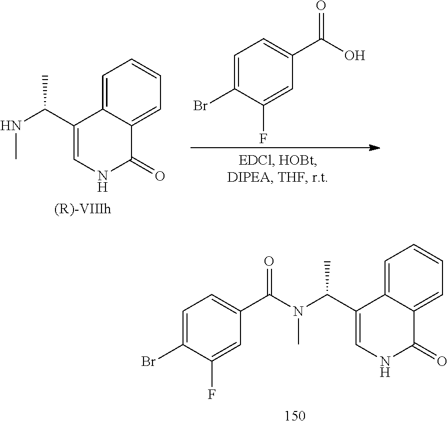

Enantiomerically pure (R)-4-bromo-3-fluoro-N-methyl-N-(1-(1-oxo-1,2-dihydroisoquinolin-4-yl)ethyl)benzamide was synthesized in a similar manner as described above (General procedure III), starting from (R)-4-(1-(methylamino)ethyl)isoquinolin-1(2H)-one ((R)-VIIIh) and 4-bromo-3-fluorobenzoic acid. LCMS: m/z found 405.2 [M+H]⁺, RT=4.33 min (Method A); ¹H NMR (400 MHz, DMSO-d₆): δ 11.42 (s, 1H), 8.27 (d, 1H), 7.82-7.74 (m, 2H), 7.64 (d, 1H), 7.55 (t, 1H), 7.35 (d, 1H), 7.20 (d, 1H), 7.06 (d, 1H), 6.05 (q, 1H), 2.44 (s, 3H), 1.53 (d, 3H).

### (R)-2-(4-Chlorophenyl)-N-methyl-N-(1-(1-oxo-1,2-dihydroisoquinolin-4-yl)ethyl)acetamide (Compound 151)

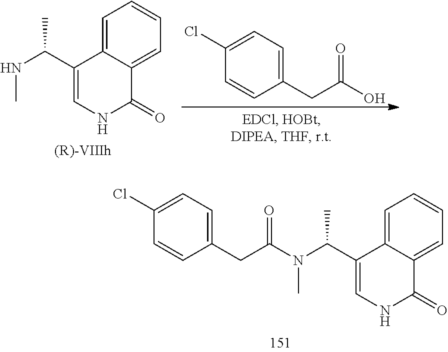

Enantiomerically pure (R)-2-(4-chlorophenyl)-N-methyl-N-(1-(1-oxo-1,2-dihydroisoquinolin-4-yl)ethyl)acetamide was synthesized in a similar manner as described above (General procedure III), starting from (R)-4-(1-(methylamino)ethyl)isoquinolin-1(2H)-one ((R)-VIIIh) and 2-(4-chlorophenyl)acetic acid. LCMS: m/z found 355.2 [M+H]⁺, RT=4.14 min (Method A); ¹H NMR (400 MHz, DMSO-d₆): δ 11.38 (s, 1H), 8.21 (d, 1H), 7.65 (t, 1H), 7.57-7.47 (m, 2H), 7.35 (d, 2H), 7.26 (d, 2H), 7.12 (d, 1H), 5.98 (q, 1H), 3.74 (d, 2H), 2.55 (s, 3H), 1.37 (d, 3H).

### (R)-N-Methyl-N-(1-(1-oxo-1,2-dihydroisoquinolin-4-yl)ethyl)indolizine-2-carboxamide (Compound 152)

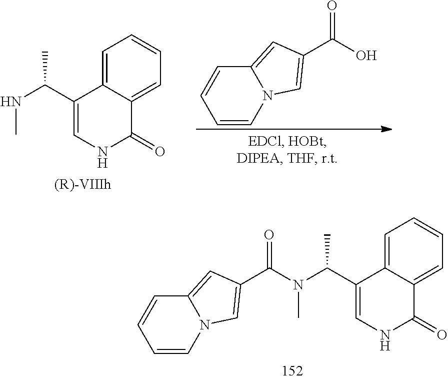

Enantiomerically pure (R)-N-methyl-N-(1-(1-oxo-1,2-dihydroisoquinolin-4-yl)ethyl)indolizine-2-carboxamide was synthesized in a similar manner as described above (General procedure III), starting from (R)-4-(1-(methylamino)ethyl)isoquinolin-1(2H)-one ((R)-VIIIh) and indolizine-2-carboxylic acid. LCMS: m/z found 346.3 [M+H]⁺, RT=3.81 min (Method A); ¹H NMR (400 MHz, DMSO-d₆): δ 11.42 (s, 1H), 8.26 (br s, 1H), 8.22 (br s, 1H), 7.83 (br s, 1H), 7.70 (br s, 1H), 7.68 (br s, 1H), 7.51 (t, 1H), 7.39 (d, 1H), 7.20 (d, 1H), 6.72 (t, 1H), 6.60-6.57 (m, 2H), 6.11 (br s, 1H), 2.75 (br s, 3H), 1.50 (br s, 3H).

### (R)-7-Fluoro-N-methyl-N-(1-(1-oxo-1,2-dihydroisoquinolin-4-yl)ethyl)indolizine-2-carboxamide (Compound 153)

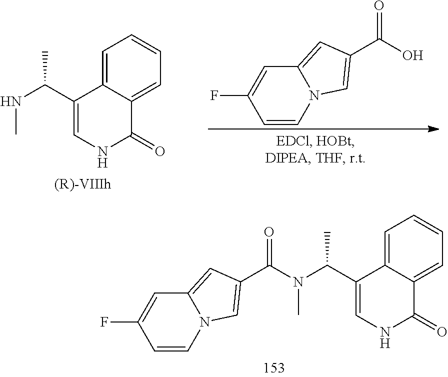 [SMILES: CN[C@H](C)c1c[nH]c(=O)c2ccccc12 ; O=C(O)c1cc2cc(F)ccn2c1 ; C[C@H](c1c[nH]c(=O)c2ccccc12)N(C)C(=O)c1cc2cc(F)ccn2c1]

Enantiomerically pure (R)-7-fluoro-N-methyl-N-(1-(1-oxo-1,2-dihydroisoquinolin-4-yl)ethyl)indolizine-2-carboxamide was synthesized in a similar manner as described above (General procedure III), starting from (R)-4-(1-(methylamino)ethyl)isoquinolin-1(2H)-one ((R)-VIIIh) and 7-fluoroindolizine-2-carboxylic acid. LCMS: m/z found 364.3 [M+H]⁺, RT=4.03 min (Method A); ¹H NMR (400 MHz, DMSO-d₆): δ 11.42 (s, 1H), 8.26 (d, 2H), 7.81 (br s, 1H), 7.70-7.49 (m, 3H), 7.20 (d, 2H), 6.66 (br s, 1H), 6.5 (br s, 1H), 6.1 (br s, 1H), 2.73 (s, 3H), 1.50 (s, 3H).

### (R)-8-Chloro-N-methyl-N-(1-(1-oxo-1,2-dihydroisoquinolin-4-yl)ethyl)indolizine-2-carboxamide (Compound 154)

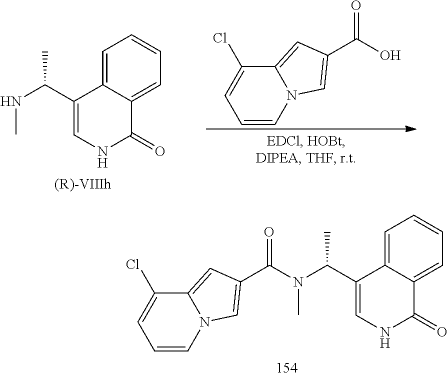 [SMILES: CN[C@H](C)c1c[nH]c(=O)c2ccccc12 ; O=C(O)c1cc2c(Cl)cccn2c1 ; C[C@H](c1c[nH]c(=O)c2ccccc12)N(C)C(=O)c1cc2c(Cl)cccn2c1]

Enantiomerically pure (R)-8-chloro-N-methyl-N-(1-(1-oxo-1,2-dihydroisoquinolin-4-yl)ethyl)indolizine-2-carboxamide was synthesized in a similar manner as described above (General procedure III), starting from (R)-4-(1-(methylamino)ethyl)isoquinolin-1(2H)-one ((R)-VIIIh) and 8-chloroindolizine-2-carboxylic acid. LCMS: m/z found 380.2 [M+H]⁺, RT=4.21 min (Method A); ¹H NMR (400 MHz, DMSO-d₆): δ 11.38 (s, 1H), 8.26 (d, 2H), 7.98 (br s, 1H), 7.71-7.64 (m, 2H), 7.51 (t, 1H), 7.21 (br s, 1H), 6.95 (br s, 1H), 6.64 (br s, 2H), 6.12 (br s, 1H), 2.76 (s, 3H), 1.51 (s, 3H).

### (R)-N-Methyl-N-(1-(1-oxo-1,2-dihydroisoquinolin-4-yl)ethyl)-1H-indole-2-carboxamide (Compound 155)

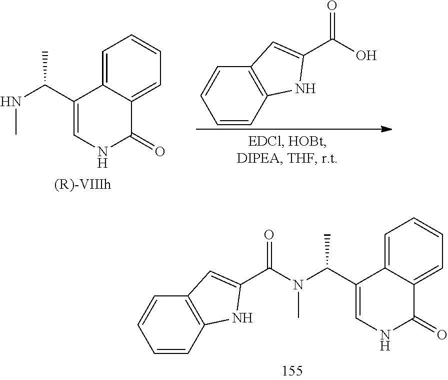

Enantiomerically pure (R)-N-methyl-N-(1-(1-oxo-1,2-dihydroisoquinolin-4-yl)ethyl)-1H-indole-2-carboxamide was synthesized in a similar manner as described above (General procedure III), starting from (R)-4-(1-(methylamino)ethyl)isoquinolin-1(2H)-one ((R)-VIIIh) and 1H-indole-2-carboxylic acid. LCMS: m/z found 346.3 [M+H]⁺, RT=4.05 min (Method A); H NMR (400 MHz, DMSO-d₆): δ 11.68 (s, 1H), 11.46 (s, 1H), 8.26 (d, 1H), 7.66-7.46 (m, 5H), 7.25 (d, 1H), 7.19 (t, 1H), 7.02 (t, 1H), 6.81 (br s, 1H), 6.15 (br s, 1H), 2.91 (s, 3H), 1.55 (d, 3H).

### (R)-4-Fluoro-N-methyl-N-(1-(1-oxo-1,2-dihydroisoquinolin-4-yl)ethyl)-1H-indole-2-carboxamide (Compound 156)

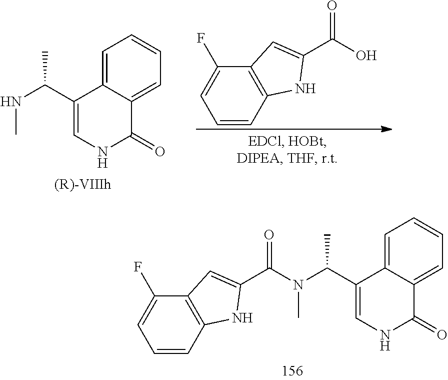

Enantiomerically pure (R)-4-fluoro-N-methyl-N-(1-(1-oxo-1,2-dihydroisoquinolin-4-yl)ethyl)-1H-indole-2-carboxamide was synthesized in a similar manner as described above (General procedure III), starting from (R)-4-(1-(methylamino)ethyl)isoquinolin-1(2H)-one ((R)-VIIIh) and 4-fluoro-1H-indole-2-carboxylic acid. LCMS: m/z found 364.3 [M+H]⁺, RT=4.20 min (Method A); ¹H NMR (400 MHz, DMSO-d₆): δ 12.03 (s, 1H), 11.47 (s, 1H), 8.26 (d, 1H), 7.68-7.63 (m, 2H), 7.50 (t, 1H), 7.30-7.25 (m, 2H), 7.20-7.15 (m, 1H), 6.87-6.78 (m, 2H), 6.15 (br s, 1H), 2.92 (s, 3H), 1.53 (s, 3H).

### (R)-5-Fluoro-N-methyl-N-(1-(1-oxo-1,2-dihydroisoquinolin-4-yl)ethyl)-1H-indole-2-carboxamide (Compound 157)

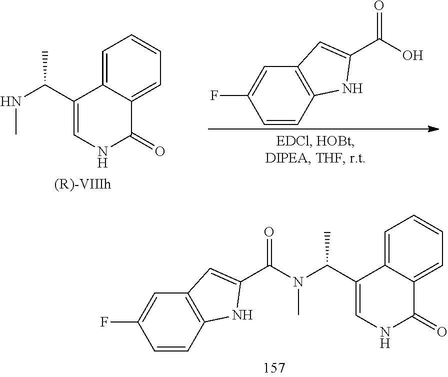 [SMILES: CN[C@H](C)c1c[nH]c(=O)c2ccccc12 ; O=C(O)c1cc2cc(F)ccc2[nH]1 ; C[C@H](c1c[nH]c(=O)c2ccccc12)N(C)C(=O)c1cc2cc(F)ccc2[nH]1]

Enantiomerically pure (R)-5-fluoro-N-methyl-N-(1-(1-oxo-1,2-dihydroisoquinolin-4-yl)ethyl)-1H-indole-2-carboxamide was synthesized in a similar manner as described above (General procedure III), starting from (R)-4-(1-(methylamino)ethyl)isoquinolin-1(2H)-one ((R)-VIIIh) and 5-fluoro-1H-indole-2-carboxylic acid. LCMS: m/z found 364.3 [M+H]⁺, RT=4.26 min (Method A); ¹H NMR (400 MHz, DMSO-d₆): δ 12.79 (s, 1H), 11.46 (s, 1H), 8.26 (d, 1H), 7.67-7.62 (m, 2H), 7.52-7.43 (m, 2H), 7.32 (d, 1H), 7.25 (d, 1H), 7.05 (t, 1H), 6.79 (br s, 1H), 6.14 (br s, 1H), 2.89 (s, 3H), 1.54 (s, 3H).

### (R)-4,6-Difluoro-N-methyl-N-(1-(1-oxo-1,2-dihydroisoquinolin-4-yl)ethyl)-1H-indole-2-carboxamide (Compound 158)

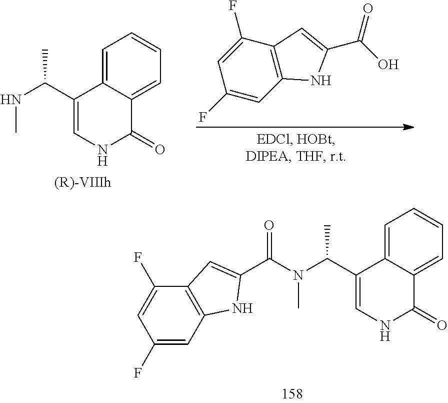 [SMILES: CN[C@H](C)c1c[nH]c(=O)c2ccccc12 ; O=C(O)c1cc2c(F)cc(F)cc2[nH]1 ; C[C@H](c1c[nH]c(=O)c2ccccc12)N(C)C(=O)c1cc2c(F)cc(F)cc2[nH]1]

Enantiomerically pure (R)-4,6-difluoro-N-methyl-N-(1-(1-oxo-1,2-dihydroisoquinolin-4-yl)ethyl)-1H-indole-2-carboxamide was synthesized in a similar manner as described above (General procedure III), starting from (R)-4-(1-(methylamino)ethyl)isoquinolin-1(2H)-one ((R)-VIIIh) and 4,6-difluoro-1H-indole-2-carboxylic acid. LCMS: m/z found 382.3 [M+H]^{m}, RT=4.51 min (Method A); ¹H NMR (400 MHz, DMSO-d₆): δ 12.11 (s, 1H), 11.46 (s, 1H), 8.26 (d, 1H), 7.67-7.62 (m, 2H), 7.50 (t, 1H), 7.26 (d, 1H), 7.05 (d, 1H), 6.90-6.85 (m, 2H), 6.14 (br s, 1H), 2.91 (s, 3H), 1.54 (s, 3H).

### (R)-5,6-Difluoro-N-methyl-N-(1-(1-oxo-1,2-dihydroisoquinolin-4-yl)ethyl)-1H-indole-2-carboxamide (Compound 159)

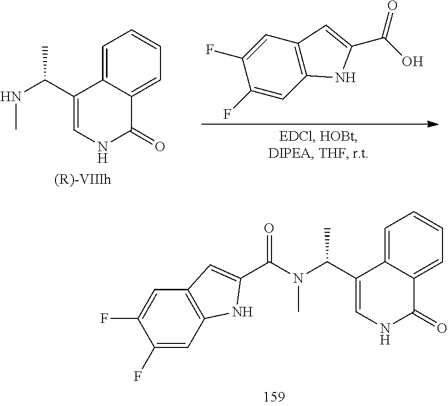 [SMILES: CN[C@H](C)c1c[nH]c(=O)c2ccccc12 ; O=C(O)c1cc2cc(F)c(F)cc2[nH]1 ; C[C@H](c1c[nH]c(=O)c2ccccc12)N(C)C(=O)c1cc2cc(F)c(F)cc2[nH]1]

Enantiomerically pure (R)-5,6-difluoro-N-methyl-N-(1-(1-oxo-1,2-dihydroisoquinolin-4-yl)ethyl)-1H-indole-2-carboxamide was synthesized in a similar manner as described above (General procedure III), starting from (R)-4-(1-(methylamino)ethyl)isoquinolin-1(2H)-one ((R)-VIIIh) and 5,6-difluoro-1H-indole-2-carboxylic acid. LCMS: m/z found 382.2 [M+H]⁺, RT=4.36 min (Method A); ¹H NMR (400 MHz, DMSO-d₆): δ 11.87 (s, 1H), 11.46 (s, 1H), 8.26 (d, 1H), 7.67-7.48 (m, 4H), 7.38-7.34 (m, 1H), 7.26 (d, 1H), 6.83 (s, 1H), 6.13 (s, 1H), 2.89 (s, 3H), 1.54 (s, 3H).

### (S)-N-Methyl-N-((R)-1-(1-oxo-1,2-dihydroisoquinolin-4-yl)ethyl)indoline-2-carboxamide (Compound 160)

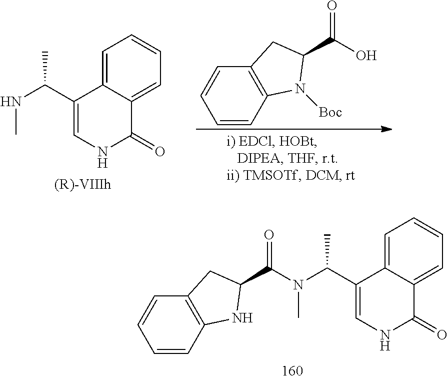

Step i. Enantiomerically pure tert-butyl (S)-2-(methyl((R)-1-(1-oxo-1,2-dihydroisoquinolin-4-yl)ethyl)carbamoyl)indoline-1-carboxylate was synthesized in a similar manner as described above (General procedure III), starting from (R)-4-(1-(methylamino)ethyl)isoquinolin-1(2H)-one ((R)-VIIIh) and (S)-1-(tert-butoxycarbonyl)indoline-2-carboxylic acid. Step ii. To a stirred solution of 85 mg (0.175 mmol, 1 eq.) of tert-butyl (S)-2-(methyl((R)-1-(1-oxo-1,2-dihydroisoquinolin-4-yl)ethyl)carbamoyl)indoline-1-carboxylate (obtained in Step i) in 5 mL of DCM was added 0.06 mL (0.35 mmol, 2 eq.) of trimethylsilyl trifluoromethanesulfonate at 0° C., and the reaction mixture was stirred for 2 h, while allowing it to warm to room temperature. After completion of the reaction the volatiles were evaporated under reduced pressure and the residue was diluted with a saturated NaHCO₃ solution (10 mL). The precipitated solid was collected by filtration and washed with water (10 mL) followed by n-pentane (10 mL) to afford 40 mg of crude product, which was purified by flash chromatography (SiO₂, eluting with 4% MeOH in dichloromethane) to afford 4.6 mg (8% yield) of (S)-N-methyl-N-((R)-1-(1-oxo-1,2-dihydroisoquinolin-4-yl)ethyl)indoline-2-carboxamide, as an off-white solid. LCMS: m/z found 348.3 [M+H]⁺, RT=2.92 min (Method A); ¹H NMR (400 MHz, DMSO-d₆): δ 11.40 (s, 1H), 8.24 (d, 1H), 7.69-7.60 (m, 1H), 7.58 (d, 1H), 7.53-7.50 (m, 1H), 7.17 (d, 1H), 6.99 (d, 1H), 6.57-6.52 (m, 1H), 6.02-5.96 (m, 1H), 5.73 (s, 1H), 4.60-4.54 (m, 1H), 3.49-3.46 (m, 1H), 3.42-3.40 (m, 1H), 3.38-3.31 (m, 1H), 3.09-3.04 (m, 1H), 2.63 (s, 3H), 1.37 (d, 3H).

### 1-(6,7-Difluoro-1-methoxyisoquinolin-4-yl)-N-methylethan-1-amine (VIa)

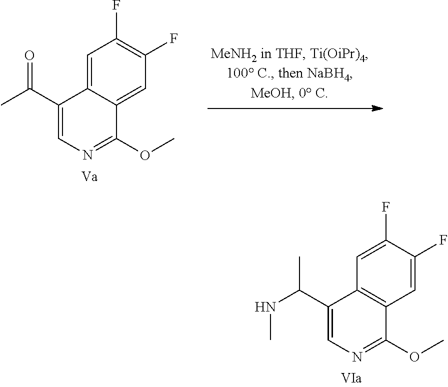 [SMILES: COc1ncc(C(C)=O)c2cc(F)c(F)cc12 ; CNC(C)c1cnc(OC)c2cc(F)c(F)cc12]

To a stirred solution of 500 mg (2.1 mmol, 1.0 eq.) of 1-(6,7-difluoro-1-methoxyisoquinolin-4-yl)ethan-1-one (Va) in 5 mL of THE was added 1.15 mL of a 2 M methylamine solution in THE (2.3 mmol, 1.5 eq.) followed by 5 mL (5 vol) of titanium isopropoxide at room temperature under a nitrogen atmosphere. The reaction mixture was stirred at 100° C. for 16 h. After imine formation (checked by LCMS after the treatment of an aliquot with NaBH₄ in methanol at room temperature for 30 min), the reaction mixture was cooled to 0° C. and diluted with methanol (5 mL) and NaBH₄ (489 mg, 12.9 mmol, 3 eq.) was added portionwise at 0° C. Stirring was continued at 0° C. for 4 h. The mixture was then diluted with water (30 mL) and extracted with ethyl acetate (3×100 mL). The combined organic extracts were dried (Na₂SO₄) and concentrated under reduced pressure to provide 350 mg of 1-(6,7-difluoro-1-methoxyisoquinolin-4-yl)-N-methylethan-1-amine (VIa), which was carried as such to the next step. LCMS m/z found 253.21 [M+H]⁺.

### 4-Bromo-N-(1-(6,7-difluoro-1-methoxyisoquinolin-4-yl)ethyl)-N-methylbenzamide (Compounds 163 and 164)

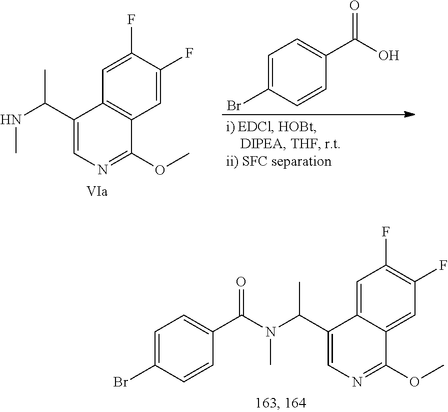 [SMILES: CNC(C)c1cnc(OC)c2cc(F)c(F)cc12 ; O=C(O)c1ccc(Br)cc1 ; COc1ncc(C(C)N(C)C(=O)c2ccc(Br)cc2)c2cc(F)c(F)cc12]

Racemic 4-bromo-N-(1-(6,7-difluoro-1-methoxyisoquinolin-4-yl)ethyl)-N-methylbenzamide was synthesized in a similar manner as described above (General procedure III), starting from 1-(6,7-difluoro-1-methoxyisoquinolin-4-yl)-N-methylethan-1-amine (VIa) and 4-bromobenzoic acid. The enantiomers were subsequently separated by chiral SFC, Column: Chiralpak-AS-H (30×250 mm), 5 μm, 75% CO₂/MeOH, Flow rate 100 g/min.

4-Bromo-N-(1-(6,7-difluoro-1-methoxyisoquinolin-4-yl)ethyl)-N-methylbenzamide—Enantiomer I (Compound 163), LCMS: m/z found 435.2 [M+H]⁺, RT=7.44 min (Method A); ¹H NMR (400 MHz, DMSO-d₆): δ 8.19 (t, 2H), 7.87 (t, 1H), 7.62 (d, 2H), 7.78 (d, 2H), 6.29 (m, 1H), 4.08 (s, 3H), 2.43 (s, 3H), 1.66 (d, 3H); Chiral analytical SFC: RT=0.91 min, Column: Chiralpak AS-3 (4.6×150 mm), 3 m, 70% CO₂/(Methanol), Flow=3.0 g/min.

4-Bromo-N-(1-(6,7-difluoro-1-methoxyisoquinolin-4-yl)ethyl)-N-methylbenzamide—Enantiomer II (Compound 164), LCMS: m/z found 435.2 [M+H]⁺, RT=7.44 min (Method A); ¹H NMR (400 MHz, DMSO-d₆): δ 8.19 (t, 2H), 7.87 (t, 1H), 7.62 (d, 2H), 7.78 (d, 2H), 6.29 (m, 1H), 4.08 (s, 3H), 2.43 (s, 3H), 1.66 (d, 3H); Chiral analytical SFC: RT=1.71 min, Column: Chiralpak AS-3 (4.6×150 mm), 3 μm, 70% CO₂/(Methanol), Flow=3.0 g/min.

### N-(1-(6,7-Difluoro-1-methoxyisoquinolin-4-yl)ethyl)-5,6-difluoro-N-methyl-1H-indole-2-carboxamide Compounds 169 and 170)

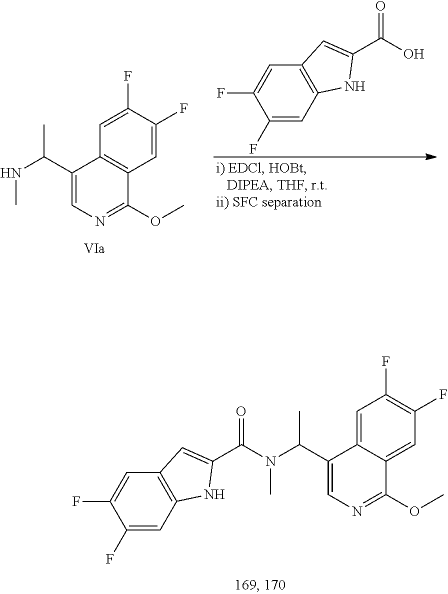

Racemic N-(1-(6,7-difluoro-1-methoxyisoquinolin-4-yl)ethyl)-5,6-difluoro-N-methyl-1H-indole-2-carboxamide was synthesized in a similar manner as described above (General procedure III), starting from 1-(6,7-difluoro-1-methoxyisoquinolin-4-yl)-N-methylethan-1-amine (VIa) and 5,6-difluoro-1H-indole-2-carboxylic acid. The enantiomers were subsequently separated by chiral SFC, Column: Chiralpak-OX-H (30×250 mm), 5 m, 70% CO₂/MeOH, Flow rate 100 g/min.

N-(1-(6,7-Difluoro-1-methoxyisoquinolin-4-yl)ethyl)-5,6-difluoro-N-methyl-1H-indole-2-carboxamide—Enantiomer I (Compound 169), LCMS: m/z found 432.3 [M+H]⁺, RT=8.13 min (Method A); ¹H NMR (400 MHz, DMSO-d₆): δ 11.90 (s, 1H), 8.24 (s, 1H), 8.14 (t, 1H), 7.89 (br s, 1H), 7.57 (t, 1H), 7.36 (t, 1H), 6.89 (s, 1H), 6.37 (m, 1H), 4.09 (s, 3H), 2.90 (s, 3H), 1.68 (s, 3H); Chiral analytical SFC: RT=1.37 min, Column: Chiralcel OX-3 (4.6×150 mm), 3 μm, 60% CO₂/(Methanol), Flow=3.0 g/min.

N-(1-(6,7-Difluoro-1-methoxyisoquinolin-4-yl)ethyl)-5,6-difluoro-N-methyl-1H-indole-2-carboxamide—Enantiomer II (Compound 170), LCMS: m/z found 432.3 [M+H]⁺, RT=8.13 min (Method A); ¹H NMR (400 MHz, DMSO-d₆): δ 11.90 (s, 1H), 8.24 (s, 1H), 8.14 (t, 1H), 7.89 (br s, 1H), 7.57 (t, 1H), 7.36 (t, 1H), 6.89 (s, 1H), 6.37 (m, 1H), 4.09 (s, 3H), 2.90 (s, 3H), 1.68 (s, 3H); Chiral analytical SFC: RT=1.66 min, Column: Chiralcel OX-3 (4.6×150 mm), 3 μm, 60% CO₂/(Methanol), Flow=3.0 g/min.

### N-(1-(6,7-Difluoro-1-methoxyisoquinolin-4-yl)ethyl)-3-fluoro-N-methyl-4-(trifluoromethyl)benzamide (Compounds 175 and 176)

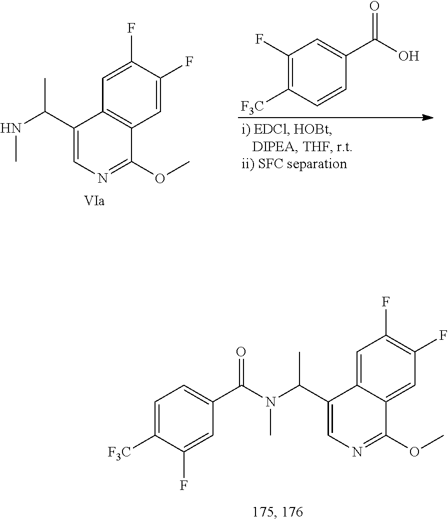 [SMILES: CNC(C)c1cnc(OC)c2cc(F)c(F)cc12 ; O=C(O)c1ccc(C(F)(F)F)c(F)c1 ; COc1ncc(C(C)N(C)C(=O)c2ccc(C(F)(F)F)c(F)c2)c2cc(F)c(F)cc12]

Racemic N-(1-(6,7-difluoro-1-methoxyisoquinolin-4-yl)ethyl)-3-fluoro-N-methyl-4-(trifluoromethyl)benzamide was synthesized in a similar manner as described above (General procedure III), starting from 1-(6,7-difluoro-1-methoxyisoquinolin-4-yl)-N-methylethan-1-amine (VIa) and 3-fluoro-4-(trifluoromethyl)benzoic acid. The enantiomers were subsequently separated by chiral SFC, Column: Lux cellulose-2 (4.6×150 mm), 3 μm, 90% CO₂/MeOH, Flow rate 90 g/min.

N-(1-(6,7-Difluoro-1-methoxyisoquinolin-4-yl)ethyl)-3-fluoro-N-methyl-4-(trifluoromethyl)benzamide—Enantiomer I (Compound 175), LCMS: m/z found 443.3 [M+H]⁺, RT=8.29 min (Method A); ¹H NMR (400 MHz, DMSO-d₆): δ 8.21-8.15 (m, 2H), 7.92-7.82 (m, 2H), 7.57 (d, 1H), 7.33 (d, 1H), 6.30 (m, 1H), 4.08 (s, 3H), 2.45 (s, 3H), 1.69 (d, 3H); Chiral analytical SFC: RT=1.32 min, Column: Lux cellulose-2 (4.6×150 mm), 3 μm, 90% CO₂/MeOH, Flow=3.0 g/min.

N-(1-(6,7-Difluoro-1-methoxyisoquinolin-4-yl)ethyl)-3-fluoro-N-methyl-4-(trifluoromethyl)benzamide—Enantiomer II (Compound 176), LCMS: m/z found 443.3 [M+H]⁺, RT=8.29 min (Method A); ¹H NMR (400 MHz, DMSO-d₆): δ 8.21-8.15 (m, 2H), 7.92-7.82 (m, 2H), 7.57 (d, 1H), 7.33 (d, 1H), 6.30 (m, 1H), 4.08 (s, 3H), 2.45 (s, 3H), 1.69 (d, 3H); Chiral analytical SFC: RT=1.58 min, Column: Lux cellulose-2 (4.6×150 mm), 3 μm, 90% CO₂/MeOH, Flow=3.0 g/min.

### 4-Bromo-N-(1-(6,7-difluoro-1-methoxyisoquinolin-4-yl)ethyl)-3-fluoro-N-methylbenzamide (Compounds 177 and 178)

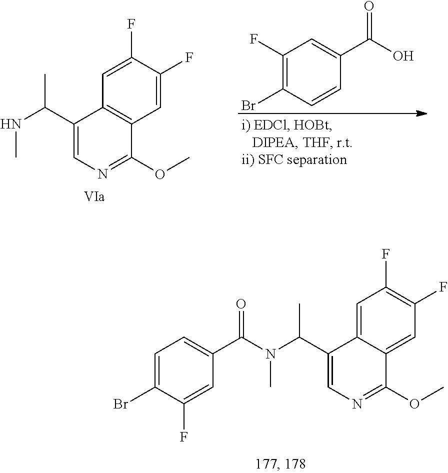 [SMILES: CNC(C)c1cnc(OC)c2cc(F)c(F)cc12 ; O=C(O)c1ccc(Br)c(F)c1 ; COc1ncc(C(C)N(C)C(=O)c2ccc(Br)c(F)c2)c2cc(F)c(F)cc12]

Racemic 4-bromo-N-(1-(6,7-difluoro-1-methoxyisoquinolin-4-yl)ethyl)-3-fluoro-N-methylbenzamide was synthesized in a similar manner as described above (General procedure III), starting from 1-(6,7-difluoro-1-methoxyisoquinolin-4-yl)-N-methylethan-1-amine (VIa) and 4-bromo-3-fluorobenzoic acid. The enantiomers were subsequently separated by chiral SFC, Column: Lux cellulose-2 (4.6×150 mm), 3 μm, 85% CO₂/MeOH, Flow rate 90 g/min.

4-Bromo-N-(1-(6,7-difluoro-1-methoxyisoquinolin-4-yl)ethyl)-3-fluoro-N-methylbenzamide—Enantiomer I (Compound 177), LCMS: m/z found 455.2 [M+H]⁺, RT=8.24 min (Method A); ¹H NMR (400 MHz, DMSO-d₆): δ 8.19-8.13 (m, 2H), 7.89-7.84 (m, 1H), 7.76 (t, 1H), 7.42 (d, 1H), 7.10 (d, 1H), 6.28 (m, 1H), 4.08 (s, 3H), 2.45 (s, 3H), 1.67 (d, 3H); Chiral analytical SFC: RT=2.47 min, Column: Lux cellulose-2 (4.6×150 mm), 3 m, 85% CO₂/MeOH, Flow=3.0 g/min.

4-Bromo-N-(1-(6,7-difluoro-1-methoxyisoquinolin-4-yl)ethyl)-3-fluoro-N-methylbenzamide—Enantiomer II (Compound 178), LCMS: m/z found 455.2 [M+H]⁺, RT=8.24 min (Method A); ¹H NMR (400 MHz, DMSO-d₆): δ 8.19-8.13 (m, 2H), 7.89-7.84 (m, 1H), 7.76 (t, 1H), 7.42 (d, 1H), 7.10 (d, 1H), 6.28 (m, 1H), 4.08 (s, 3H), 2.45 (s, 3H), 1.67 (d, 3H); Chiral analytical SFC: RT=3.06 min, Column: Lux cellulose-2 (4.6×150 mm), 3 μm, 85% CO₂/MeOH, Flow=3.0 g/min.

### N-(1-(6,7-Difluoro-1-methoxyisoquinolin-4-yl)ethyl)-8-fluoro-N-methylindolizine-2-carboxamide (Compounds 179 and 180)

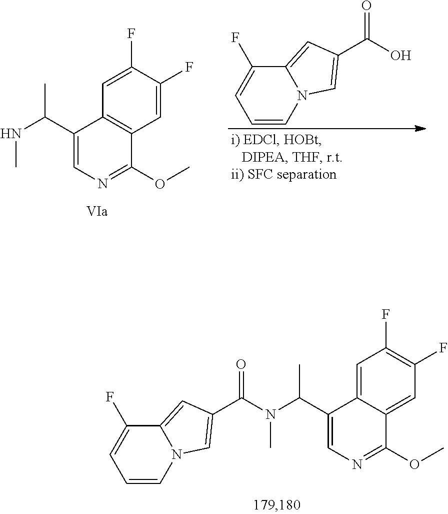 [SMILES: CNC(C)c1cnc(OC)c2cc(F)c(F)cc12 ; O=C(O)c1cc2c(F)cccn2c1 ; COc1ncc(C(C)N(C)C(=O)c2cc3c(F)cccn3c2)c2cc(F)c(F)cc12]

Racemic N-(1-(6,7-difluoro-1-methoxyisoquinolin-4-yl)ethyl)-8-fluoro-N-methylindolizine-2-carboxamide was synthesized in a similar manner as described above (General procedure III), starting from 1-(6,7-difluoro-1-methoxyisoquinolin-4-yl)-N-methylethan-1-amine (VIa) and 8-fluoroindolizine-2-carboxylic acid. The enantiomers were subsequently separated by chiral SFC, Column: Lux cellulose-2 (4.6×150 mm), 3 μm, 85% CO₂/MeOH, Flow rate 90 g/min.

N-(1-(6,7-Difluoro-1-methoxyisoquinolin-4-yl)ethyl)-8-fluoro-N-methylindolizine-2-carboxamide—Enantiomer I (Compound 179), LCMS: m/z found 414.3 [M+H]⁺, RT=7.98 min (Method A); ¹H NMR (400 MHz, DMSO-d₆): δ 8.21 (s, 1H), 8.17-8.12 (m, 2H), 8.05 (s, 1H), 7.89 (br s, 1H), 6.73 (s, 1H), 76.63-6.61 (m, 2H), 6.35 (br s, 1H), 4.09 (s, 3H), 2.76 (s, 3H), 1.67 (d, 3H); Chiral analytical SFC: RT=5.13 min, Column: Lux cellulose-2 (4.6×150 mm), 3 μm 85% CO₂/MeOH, Flow=3.0 g/min.

N-(1-(6,7-Difluoro-1-methoxyisoquinolin-4-yl)ethyl)-8-fluoro-N-methylindolizine-2-carboxamide—Enantiomer II (Compound 180), LCMS: m/z found 414.3 [M+H]⁺, RT=7.98 min (Method A); ¹H NMR (400 MHz, DMSO-d₆): δ 8.21 (s, 1H), 8.17-8.12 (m, 2H), 8.05 (s, 1H), 7.89 (br s, 1H), 6.73 (s, 1H), 76.63-6.61 (m, 2H), 6.35 (br s, 1H), 4.09 (s, 3H), 2.76 (s, 3H), 1.67 (d, 3H); Chiral analytical SFC: RT=6.35 min, Column: Lux cellulose-2 (4.6×150 mm), 3 μm, 85% CO₂/MeOH, Flow=3.0 g/min.

### (2S)-N-(1-(6,7-Difluoro-1-methoxyisoquinolin-4-yl)ethyl)-N-methylindoline-2-carboxamide (Compounds 181 and 182)

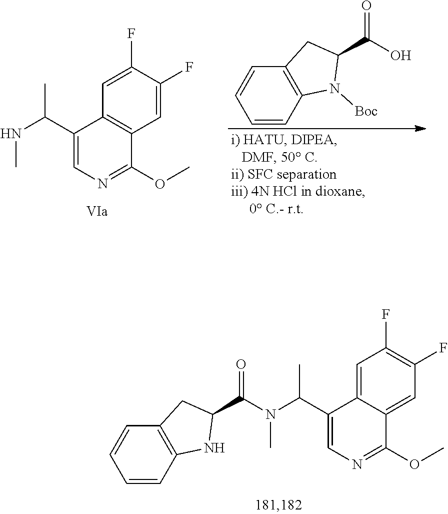 [SMILES: CNC(C)c1cnc(OC)c2cc(F)c(F)cc12 ; CC(C)(C)OC(=O)N1c2ccccc2C[C@H]1C(=O)O ; COc1ncc(C(C)N(C)C(=O)[C@@H]2Cc3ccccc3N2)c2cc(F)c(F)cc12]

Step i. Diastereomeric mixture of tert-butyl (2S)-2-((1-(6,7-difluoro-1-methoxyisoquinolin-4-yl)ethyl)(methyl)carbamoyl)indoline-1-carboxylate was synthesized in a similar manner as described above (General procedure II, except conducted at 50° C.), starting from 1-(6,7-difluoro-1-methoxyisoquinolin-4-yl)-N-methylethan-1-amine (VIa) and (S)-1-(tert-butoxycarbonyl)indoline-2-carboxylic acid.

Step ii. The diastereoisomers were separated by chiral SFC, Column: Chiralpak IC-3 (30×250 mm), 5 μm, 70% CO₂/MeOH, Flow rate 100 g/min. Step iii. Each isolated diastereoisomer of tert-butyl (2S)-2-((1-(6,7-difluoro-1-methoxyisoquinolin-4-yl)ethyl)(methyl)carbamoyl)indoline-1-carboxylate, dissolved in dioxane, was treated with 4 M HCl in dioxane (5 vol) at 0° C. and the resulting reaction mixtures were stirred at room temperature for 10 h. After completion, the volatiles were removed under reduced pressure. The resulting residues were each taken in saturated NaHCO₃ solution and stirred for 10 min. The precipitated solids were collected by filtration. The obtained crude materials were triturated with diethyl ether and filtered. The solids were dried under vacuum and lyophilized to obtain the individual diastereoisomers of the final product.

(2S)-N-(1-(6,7-Difluoro-1-methoxyisoquinolin-4-yl)ethyl)-N-methylindoline-2-carboxamide—Diastereoisomer I (Compound 181), LCMS: m/z found 398.3 [M+H]⁺, RT=4.83 min (Method A); ¹H NMR (400 MHz, DMSO-d₆): δ 8.16-8.11 (m, 2H), 7.83-7.78 (m, 1H), 7.09-6.91 (m, 2H), 6.57-6.53 (t, 2H), 6.20-6.15 (m, 1H), 5.74 (bs, 1H), 4.62-4.59 (m, 1H) 4.11 (s, 3H), 3.31-3.29 (m, 1H), 3.15-3.10 (m, 1H), 2.64 (s, 3H), 1.54 (d, 3H); Chiral analytical SFC: RT=1.53 min, Column: Chiralpak IA-3 (4.6×150 mm), 3 μm, 80% CO₂/(0.5% of DEA in Methanol), Flow=3.0 g/min.

(2S)-N-(1-(6,7-Difluoro-1-methoxyisoquinolin-4-yl)ethyl)-N-methylindoline-2-carboxamide—Diastereoisomer II (Compound 182), LCMS: m/z found 398.3 [M+H]⁺, RT=4.85 min (Method A); ¹H NMR (400 MHz, DMSO-d₆): δ 8.55-8.11 (t, 2H), 7.55-7.50 (m, 1H), 6.96-6.92 (m, 2H), 6.60-6.53 (m, 2H), 6.16-6.11 (m, 1H), 5.77 (bs, 1H), 4.66-4.62 (m, 1H), 4.08 (s, 3H), 3.31-3.18 (m, 1H), 2.78-2.73 (m, 1H), 2.58 (s, 3H), 1.55 (d, 3H); Chiral analytical SFC: RT=1.95 min, Column: Chiralpak IA-3 (4.6×150 mm), 3 μm, 80% CO₂/(0.5% of DEA in Methanol), Flow=3.0 g/min.

### Synthesis of Intermediate XVIIIa

### 4-Bromo-6,7-difluoro-2-methylisoquinolin-1(2H)-one (XVIa)

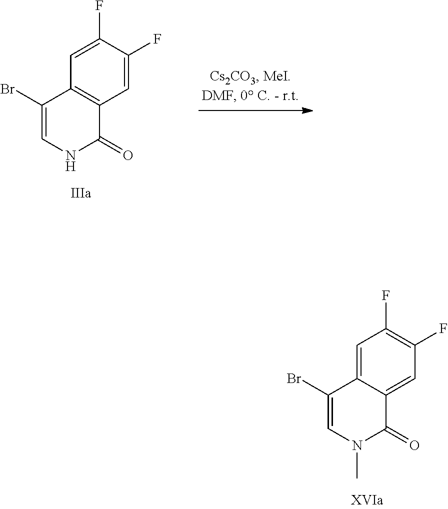

To a stirred solution of 3 g (11.6 mmol, 1 eq.) of 4-bromo-6,7-difluoroisoquinolin-1(2H)-one (IIa) in 30 mL of DMF at 0° C. were added 11.3 g (34.7 mmol, 3 eq.) of cesium carbonate and 1.2 mL (17.4 mmol, 1.5 eq.) of iodomethane under a nitrogen atmosphere. The reaction mixture was stirred at room temperature for 16 h. After completion of reaction, the mixture was diluted with 200 mL of ice-cold water and stirred for a further 15 min. The precipitated solid was collected by filtration and washed with water (10 mL), then dried under vacuum. The crude solid was purified by silica gel column chromatography (eluting with 50% ethyl acetate in petroleum ether) to provide 0.65 g (20% yield) of 4-bromo-6,7-difluoro-2-methylisoquinolin-1(2H)-one (XVIa). LCMS m/z found 273.93 [M+H]⁺, RT=1.84 min, (Method E); ¹H NMR (400 MHz, DMSO-d₆): δ 8.21-8.16 (m, 1H), 8.05 (s, 1H), 7.75-7.70 (m, 1H), 3.51 (s, 3H).

### 4-Acetyl-6,7-difluoro-2-methylisoquinolin-1(2H)-one (XVIIa)

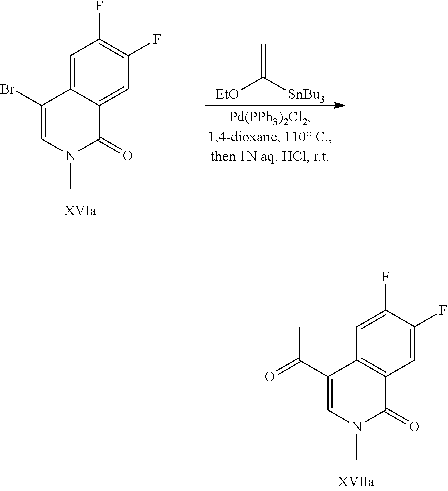

To a stirred solution of 0.65 g (2.4 mmol, 1.0 eq.) of 4-bromo-6,7-difluoro-2-methylisoquinolin-1(2H)-one (XVIa) in 10 mL of 1,4-dioxane was added 2.15 g (5.95 mmol, 2.5 eq.) of tributyl(1-ethoxyvinyl)stannane at room temperature and the system was purged with nitrogen gas for 5 min. To this mixture was added 0.167 g (0.23 mmol, 0.1 eq.) of Pd(PPh₃)₂Cl₂ at room temperature and the mixture was heated to 110° C. for 16 h. After completion of reaction, the mixture was cooled to room temperature, treated with 3 mL of 1 M aqueous HCl solution and stirred at room temperature for a further 2 h. The reaction mixture was filtered through a CELITE® and the pad was washed with 1,4-dioxane (10 mL). The filtrate was concentrated under reduced pressure to afford the crude product, which was purified by trituration with n-pentane (20 mL), filtered and dried under vacuum to afford 0.45 g (79% yield) of 4-acetyl-6,7-difluoro-2-methylisoquinolin-1(2H)-one (XVIIa), as an off-white solid. LCMS m/z found 238.15 [M+H]⁺, RT=2.09 min (Method E); ¹H NMR (400 MHz, DMSO-d₆): δ 8.92-8.86 (m, 1H), 8.67 (s, 1H), 8.18-8.12 (m, 1H), 3.63 (s, 3H), 2.55 (s, 3H).

### 6,7-Difluoro-2-methyl-4-(1-(methylamino)ethyl)isoquinolin-1(2H)-one (XVIIIa)

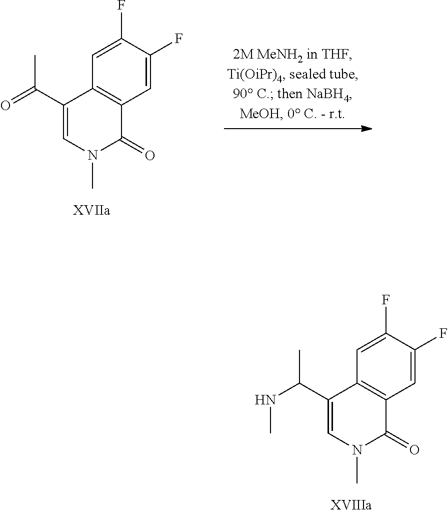 [SMILES: CC(=O)c1cn(C)c(=O)c2cc(F)c(F)cc12 ; CNC(C)c1cn(C)c(=O)c2cc(F)c(F)cc12]

To a stirred solution of 250 mg (1.1 mmol, 1.0 eq.) of 4-acetyl-6,7-difluoro-2-methylisoquinolin-1(2H)-one (XVIIa) in 2 mL of THF under a nitrogen atmosphere was added 0.8 mL (1.6 mmol, 1.5 eq.) of a 2 M methylamine solution in THF followed by 2 mL of titanium isopropoxide at room temperature. The reaction mixture was heated to 90° C. for 2 h. After imine formation (checked by LCMS after the treatment of an aliquot with NaBH₄ in methanol at room temperature for 30 min), the mixture was cooled to 0° C. and diluted with 2 mL of methanol. To this mixture, 80 mg (2.1 mmol, 2 eq.) of NaBH₄ was added portionwise at 0° C. and the reaction was stirred at room temperature for a further 2 h. After completion of reaction, the mixture was diluted with 1 mL of brine and the resultant heterogeneous mixture was slurried with 30 mL of methanol and stirred for 1 h. The mixture was filtered through a CELITE® and the pad was washed with 10 mL of methanol. The combined filtrate was concentrated under reduced pressure to afford 240 mg of 6,7-difluoro-2-methyl-4-(1-(methylamino)ethyl)isoquinolin-1(2H)-one (XVIIIa) as an off-white solid, which was taken as such for next step. LCMS m/z found 253.09 [M+H]⁺, RT=1.11 min (Method D), ¹H NMR (300 MHz, DMSO-d₆): δ 8.21-8.15 (m, 1H), 8.14-8.08 (m, 1H), 7.47 (s, 1H), 3.89-3.82 (m, 1H), 3.51 (s, 3H), 2.21 (s, 3H), 1.32 (d, 3H).

### N-(1-(6,7-Difluoro-2-methyl-1-oxo-1,2-dihydroisoquinolin-4-yl)ethyl)-3-fluoro-N-methyl-4-(trifluoromethyl)benzamide (Compounds 165 and 166)

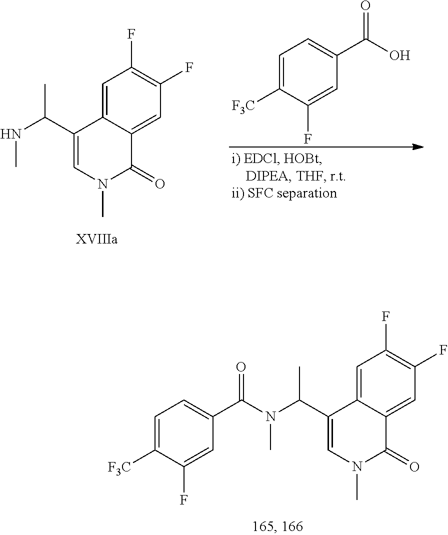 [SMILES: CNC(C)c1cn(C)c(=O)c2cc(F)c(F)cc12 ; O=C(O)c1ccc(C(F)(F)F)c(F)c1 ; CC(c1cn(C)c(=O)c2cc(F)c(F)cc12)N(C)C(=O)c1ccc(C(F)(F)F)c(F)c1]

Racemic N-(1-(6,7-difluoro-2-methyl-1-oxo-1,2-dihydroisoquinolin-4-yl)ethyl)-3-fluoro-N-methyl-4-(trifluoromethyl)benzamide was synthesized in a similar manner as described above (General procedure III), starting from 6,7-difluoro-2-methyl-4-(1-(methylamino)ethyl)isoquinolin-1(2H)-one (XVIIIa) and 3-fluoro-4-(trifluoromethyl)benzoic acid. The enantiomers were subsequently separated by chiral SFC, Column: Lux Cellulose-2 (21×250 mm), 5 μm 70% CO₂/MeOH, Flow rate 100 g/min.

N-(1-(6,7-Difluoro-2-methyl-1-oxo-1,2-dihydroisoquinolin-4-yl)ethyl)-3-fluoro-N-methyl-4-(trifluoromethyl)benzamide—Enantiomer I (Compound 165), LCMS: m/z found 443.3 [M+H]⁺, RT=6.75 min (Method A); ¹H NMR (400 MHz, DMSO-d₆): δ 8.18 (t, 1H), 7.85 (s, 1H), 7.67-7.55 (m, 3H), 7.32 (d, 1H), 6.07 (m, 1H), 3.57 (s, 3H), 2.49 (s, 3H), 1.56 (d, 3H); Chiral analytical SFC: RT=1.42 min, Column: Chiralcel OX-3 (4.6×150 mm), 3 m, 80% CO₂/(0.5% DEA in Methanol), Flow=3.0 g/min.

N-(1-(6,7-Difluoro-2-methyl-1-oxo-1,2-dihydroisoquinolin-4-yl)ethyl)-3-fluoro-N-methyl-4-(trifluoromethyl)benzamide—Enantiomer II (Compound 166), LCMS: m/z found 443.3 [M+H]⁺, RT=6.75 min (Method A); ¹H NMR (400 MHz, DMSO-d₆): δ 8.18 (t, 1H), 7.85 (s, 1H), 7.67-7.55 (m, 3H), 7.32 (d, 1H), 6.07 (m, 1H), 3.57 (s, 3H), 2.49 (s, 3H), 1.56 (d, 3H); Chiral analytical SFC: RT=4.37 min, Column: Chiralcel OX-3 (4.6×150 mm), 3 m, 80% CO₂/(0.5% DEA in Methanol), Flow=3.0 g/min.

### 4-Bromo-N-(1-(6,7-difluoro-2-methyl-1-oxo-1,2-dihydroisoquinolin-4-yl)ethyl)-3-fluoro-N-methylbenzamide (Compound 161 and 162)

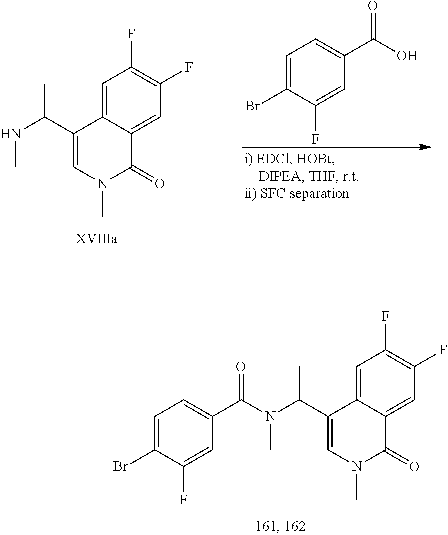 [SMILES: CNC(C)c1cn(C)c(=O)c2cc(F)c(F)cc12 ; O=C(O)c1ccc(Br)c(F)c1 ; CC(c1cn(C)c(=O)c2cc(F)c(F)cc12)N(C)C(=O)c1ccc(Br)c(F)c1]

Racemic 4-bromo-N-(1-(6,7-difluoro-2-methyl-1-oxo-1,2-dihydroisoquinolin-4-yl)ethyl)-3-fluoro-N-methylbenzamide was synthesized in a similar manner as described above (General procedure III), starting from 6,7-difluoro-2-methyl-4-(1-(methylamino)ethyl)isoquinolin-1(2H)-one (XVIIIa) and 4-bromo-3-fluorobenzoic acid acid. The enantiomers were subsequently separated by chiral SFC, Column: Lux Cellulose-2 (21×250 mm), 5 m, 70% CO₂/MeOH, Flow rate 100 g/min.

4-Bromo-N-(1-(6,7-difluoro-2-methyl-1-oxo-1,2-dihydroisoquinolin-4-yl)ethyl)-3-fluoro-N-methylbenzamide—Enantiomer I (Compound 161), LCMS: m/z found 455.2 [M+H]⁺, RT=6.64 min (Method A); ¹H NMR (400 MHz, DMSO-d₆): δ 8.17 (t, 1H), 7.78 (t, 1H), 7.66-7.58 (m, 2H), 7.41 (d, 1H), 7.09 (d, 1H), 6.04 (m, 1H), 3.58 (s, 3H), 2.50 (s, 3H), 1.55 (d, 3H); Chiral analytical SFC: RT=2.23 min, Column: Chiralcel OX-3 (4.6×150 mm), 3 μm, 70% CO₂/(Methanol), Flow=3.0 g/min.

4-Bromo-N-(1-(6,7-difluoro-2-methyl-1-oxo-1,2-dihydroisoquinolin-4-yl)ethyl)-3-fluoro-N-methylbenzamide—Enantiomer II (Compound 162), LCMS: m/z found 455.2 [M+H]⁺, RT=6.64 min (Method A); ¹H NMR (400 MHz, DMSO-d₆): δ 8.17 (t, 1H), 7.78 (t, 1H), 7.66-7.58 (m, 2H), 7.41 (d, 1H), 7.09 (d, 1H), 6.04 (m, 1H), 3.58 (s, 3H), 2.50 (s, 3H), 1.55 (d, 3H); Chiral analytical SFC: RT=2.61 min, Column: Chiralcel OX-3 (4.6×150 mm), 3 μm, 70% CO₂/(Methanol), Flow=3.0 g/min.

### 4-Bromo-N-(1-(6,7-difluoro-2-methyl-1-oxo-1,2-dihydroisoquinolin-4-yl)ethyl)-N-methylbenzamide (Compounds 167 and 168)

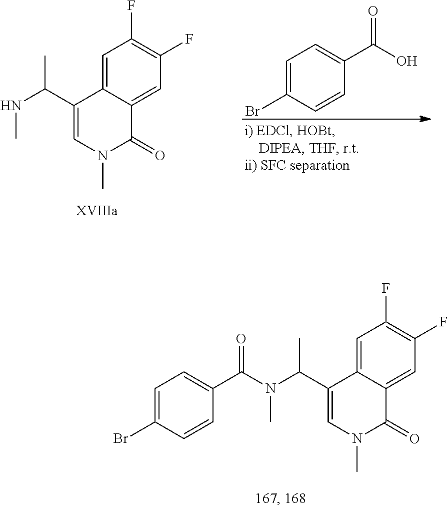 [SMILES: CNC(C)c1cn(C)c(=O)c2cc(F)c(F)cc12 ; O=C(O)c1ccc(Br)cc1 ; CC(c1cn(C)c(=O)c2cc(F)c(F)cc12)N(C)C(=O)c1ccc(Br)cc1]

Racemic 4-bromo-N-(1-(6,7-difluoro-2-methyl-1-oxo-1,2-dihydroisoquinolin-4-yl)ethyl)-N-methylbenzamide was synthesized in a similar manner as described above (General procedure III), starting from 6,7-difluoro-2-methyl-4-(1-(methylamino)ethyl)isoquinolin-1(2H)-one (XVIIIa) and 4-bromobenzoic acid. The enantiomers were subsequently separated by chiral SFC, Column: Lux Cellulose-2 (21×250 mm), 5 m, 70% CO₂/MeOH, Flow rate 100 g/min.

4-Bromo-N-(1-(6,7-difluoro-2-methyl-1-oxo-1,2-dihydroisoquinolin-4-yl)ethyl)-N-methylbenzamide—Enantiomer I (Compound 167), LCMS: m/z found 437.2 [M+H]⁺, RT=6.53 min (Method A); ¹H NMR (400 MHz, DMSO-d₆): δ 8.17 (t, 1H), 7.64-7.59 (m, 4H), 7.28-7.26 (m, 2H), 6.05 (m, 1H), 3.57 (s, 3H), 2.47 (s, 3H), 1.53 (d, 3H); Chiral analytical SFC: RT=4.90 min, Column: Chiralcel OX-3 (4.6×150 mm), 3 μm, 80% CO₂/(0.5% DEA in Methanol), Flow=3.0 g/min.

4-Bromo-N-(1-(6,7-difluoro-2-methyl-1-oxo-1,2-dihydroisoquinolin-4-yl)ethyl)-N-methylbenzamide—Enantiomer II (Compound 168), LCMS: m/z found 437.2 [M+H]⁺, RT=6.53 min (Method A); ¹H NMR (400 MHz, DMSO-d₆): δ 8.17 (t, 1H), 7.64-7.59 (m, 4H), 7.28-7.26 (m, 2H), 6.05 (m, 1H), 3.57 (s, 3H), 2.47 (s, 3H), 1.53 (d, 3H); Chiral analytical SFC: RT=5.81 min, Column: Chiralcel OX-3 (4.6×150 mm), 3 μm, 80% CO₂/(0.5% DEA in Methanol), Flow=3.0 g/min.

### N-(1-(6,7-Difluoro-2-methyl-1-oxo-1,2-dihydroisoquinolin-4-yl)ethyl)-8-fluoro-N-methylindolizine-2-carboxamide (Compounds 171 and 172)

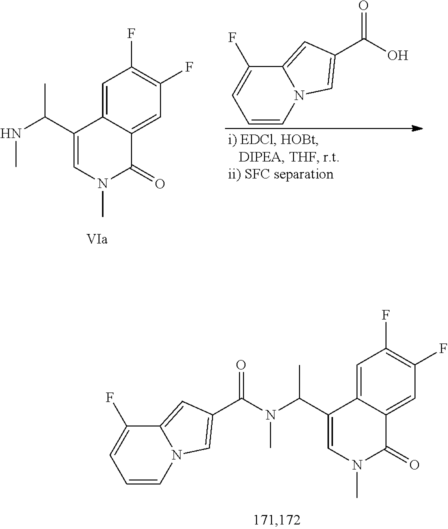 [SMILES: CNC(C)c1cn(C)c(=O)c2cc(F)c(F)cc12 ; O=C(O)c1cc2c(F)cccn2c1 ; CC(c1cn(C)c(=O)c2cc(F)c(F)cc12)N(C)C(=O)c1cc2c(F)cccn2c1]

Racemic N-(1-(6,7-difluoro-2-methyl-1-oxo-1,2-dihydroisoquinolin-4-yl)ethyl)-8-fluoro-N-methylindolizine-2-carboxamide was synthesized in a similar manner as described above (General procedure III), starting from 6,7-difluoro-2-methyl-4-(1-(methylamino)ethyl)isoquinolin-1(2H)-one (XVIIIa) and 8-fluoroindolizine-2-carboxylic acid. The enantiomers were subsequently separated by chiral SFC, Column: Chiralpak-IC-3 (30×250 mm), 5 m, 60% CO₂/MeOH, Flow rate 100 g/min.

N-(1-(6,7-Difluoro-2-methyl-1-oxo-1,2-dihydroisoquinolin-4-yl)ethyl)-8-fluoro-N-methylindolizine-2-carboxamide—Enantiomer I (Compound 171), LCMS: m/z found 414.3 [M+H]⁺, RT=7.31 min (Method A); ¹H NMR (400 MHz, DMSO-d₆): δ 8.18-8.04 (m, 3H), 7.66 (br s, 2H), 6.74 (s, 1H), 6.63-6.61 (m, 2H), 6.11 (br s, 1H), 3.59 (s, 3H), 2.80 (s, 3H), 1.54 (br s, 3H); Chiral analytical SFC: RT=4.49 min, Column: Chiralpak IC-3 (4.6×150 mm), 3 μm, 60% CO₂/(Methanol), Flow=3.0 g/min.

N-(1-(6,7-Difluoro-2-methyl-1-oxo-1,2-dihydroisoquinolin-4-yl)ethyl)-8-fluoro-N-methylindolizine-2-carboxamide—Enantiomer II (Compound 172), LCMS: m/z found 414.3 [M+H]⁺, RT=7.31 min (Method A); ¹H NMR (400 MHz, DMSO-d₆): δ 8.18-8.04 (m, 3H), 7.66 (br s, 2H), 6.74 (s, 1H), 6.63-6.61 (m, 2H), 6.11 (br s, 1H), 3.59 (s, 3H), 2.80 (s, 3H), 1.54 (br s, 3H); Chiral analytical SFC: RT=5.95 min, Column: Chiralpak IC-3 (4.6×150 mm), 3 μm, 60% CO₂/(Methanol), Flow=3.0 g/min.

### N-(1-(6,7-Difluoro-2-methyl-1-oxo-1,2-dihydroisoquinolin-4-yl)ethyl)-5,6-difluoro-N-methyl-1H-indole-2-carboxamide (Compounds 173 and 174)

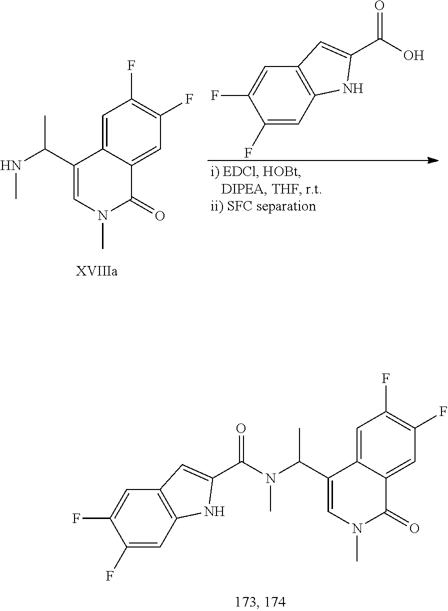

Racemic N-(1-(6,7-difluoro-2-methyl-1-oxo-1,2-dihydroisoquinolin-4-yl)ethyl)-5,6-difluoro-N-methyl-1H-indole-2-carboxamide was synthesized in a similar manner as described above (General procedure III), starting from 6,7-difluoro-2-methyl-4-(1-(methylamino)ethyl)isoquinolin-1(2H)-one (XVIIIa) and 5,6-difluoro-1H-indole-2-carboxylic acid. The enantiomers were subsequently separated by chiral SFC, Column: Chiralpak-IC-3 (30×250 mm), 5 m, 60% CO₂/MeOH, Flow rate 110 g/min.

N-(1-(6,7-Difluoro-2-methyl-1-oxo-1,2-dihydroisoquinolin-4-yl)ethyl)-5,6-difluoro-N-methyl-1H-indole-2-carboxamide—Enantiomer I (Compound 173), LCMS: m/z found 432.3 [M+H]⁺, RT=7.52 min (Method A); ¹H NMR (400 MHz, DMSO-d₆): δ 11.89 (s, 1H), 8.15 (t, 1H), 7.70 (s, 1H), 7.64-7.57 (m, 2H), 7.39-7.35 (m, 1H), 6.91 (s, 1H), 6.14 (m, 1H), 3.60 (s, 3H), 2.94 (s, 3H), 1.55 (d, 3H); Chiral analytical SFC: RT=2.35 min, Column: Chiralpak IC-3 (4.6×150 mm), 3 m, 60% CO₂/(Methanol), Flow=3.0 g/min.

N-(1-(6,7-Difluoro-2-methyl-1-oxo-1,2-dihydroisoquinolin-4-yl)ethyl)-5,6-difluoro-N-methyl-1H-indole-2-carboxamide—Enantiomer II (Compound 174), LCMS: m/z found 432.3 [M+H]⁺, RT=7.52 min (Method A); ¹H NMR (400 MHz, DMSO-d₆): δ 11.89 (s, 1H), 8.15 (t, 1H), 7.70 (s, 1H), 7.64-7.57 (m, 2H), 7.39-7.35 (m, 1H), 6.91 (s, 1H), 6.14 (m, 1H), 3.60 (s, 3H), 2.94 (s, 3H), 1.55 (d, 3H); Chiral analytical SFC: RT=3.72 min, Column: Chiralpak IC-3 (4.6×150 mm), 3 μm, 60% CO₂/(Methanol), Flow=3.0 g/min.

### (2S)-N-(1-(6,7-Difluoro-2-methyl-1-oxo-1,2-dihydroisoquinolin-4-yl)ethyl)-N-methylindoline-2-carboxamide (Compounds 183 and 184)

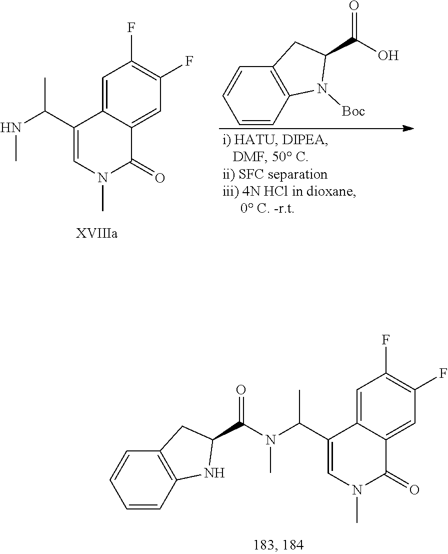 [SMILES: CNC(C)c1cn(C)c(=O)c2cc(F)c(F)cc12 ; CC(C)(C)OC(=O)N1c2ccccc2C[C@H]1C(=O)O ; CC(c1cn(C)c(=O)c2cc(F)c(F)cc12)N(C)C(=O)[C@@H]1Cc2ccccc2N1]

Step i. Diastereomeric mixture of tert-butyl (2S)-2-((1-(6,7-difluoro-2-methyl-1-oxo-1,2-dihydroisoquinolin-4-yl)ethyl)(methyl)carbamoyl)indoline-1-carboxylate was synthesized in a similar manner as described above (General procedure II, except conducted at 50° C.), starting from 6,7-difluoro-2-methyl-4-(1-(methylamino)ethyl)isoquinolin-1(2H)-one (XVIIIa) and (S)-1-(tert-butoxycarbonyl)indoline-2-carboxylic acid. Step ii. The diastereoisomers were separated by chiral SFC, Column: DCPAKP4VP (30×250) mm, 5μ, 85% CO₂/MeOH, Flow rate 65 g/min. Step iii. Each isolated diastereoisomer of tert-butyl (2S)-2-((1-(6,7-difluoro-2-methyl-1-oxo-1,2-dihydroisoquinolin-4-yl)ethyl)(methyl)carbamoyl)indoline-1-carboxylate dissolved in dioxane was treated with 4 M HCl in dioxane (5 vol) at 0° C. and the resulting reaction mixtures were stirred at room temperature for 10 h. After completion, the volatiles were removed under reduced pressure. The resulting residues were each taken in saturated NaHCO₃ solution (10 mL) and stirred for 10 min. The precipitated solids were collected by filtration. The obtained crude materials were triturated with diethyl ether and filtered. The solids were dried under vacuum and lyophilized to obtain the individual diastereoisomers of the final product.

(2S)-N-(1-(6,7-Difluoro-2-methyl-1-oxo-1,2-dihydroisoquinolin-4-yl)ethyl)-N-methylindoline-2-carboxamide—Diastereoisomer I (Compound 183), LCMS: m/z found 398.3 [M+H]⁺, RT=4.20 min (Method A); ¹H NMR (400 MHz, DMSO-d₆): δ 8.15 (m, 1H), 7.61-7.52 (m, 2H), 7.01-6.91 (m, 2H), 6.55 (m, 2H), 5.94 (m, 1H), 5.75 (br s, 1H), 4.63-4.60 (m, 1H), 3.58 (s, 3H), 3.31-3.29 (m, 1H), 3.11 (m, 1H), 2.68 (s, 3H), 1.41 (d, 3H); Chiral analytical SFC: RT=1.48 min, Column: Chiralpak OX-3 (4.6×150 mm), 3 m, 80% CO₂/(Methanol), Flow=3.0 g/min.

(2S)-N-(1-(6,7-Difluoro-2-methyl-1-oxo-1,2-dihydroisoquinolin-4-yl)ethyl)-N-methylindoline-2-carboxamide—Diastereoisomer II (Compound 184), LCMS: m/z found 398.3 [M+H]⁺, RT=5.87 min (Method A); ¹H NMR (400 MHz, DMSO-d₆): δ 8.15 (m, 1H), 7.60 (s, 1H), 7.26 (m, 1H), 6.94 (m, 2H), 6.61-6.52 (m, 2H), 5.90 (m, 1H), 5.76 (br s, 1H), 4.63 (m, 1H), 3.58 (s, 3H), 3.25 (in, 11-1), 2.78 (in, 1H1), 2.64 (s, 311), 1.43 (d, 3H); Chiral analytical SFC: RT=2.18 min, Column: Chiralpak OX-3 (4.6×150 mm), 3 μm, 8000 CO₂/(Methanol), Flow=3.0 g/min.

### Example 2: Biological Results

Representative compounds of the disclosure were tested for their abilities to inhibit formation of relaxed circular DNA (rcDNA) in a HepDET9 assay, as described elsewhere herein. Results are illustrated in Table 1.

TABLE 1

| | | DE-19bDNA |
|---|---|---|
| No. | Nomenclature | EC₅₀ (μM) |
| 1 | 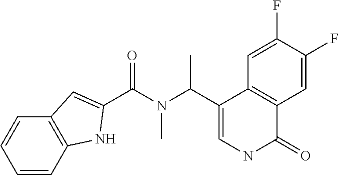 | 2.5 |
| | N-(1-(6,7-difluoro-1-oxo-1,2-dihydroisoquinolin-4-yl) | |
| | ethyl)-N-methyl-1H-indole-2-carboxamide | |
| | enantiomer I | |
| 2 | 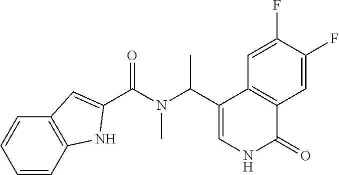 [SMILES: CC(c1c[nH]c(=O)c2cc(F)c(F)cc12)N(C)C(=O)c1cc2ccccc2[nH]1] | 0.02 |
| | N-(1-(6,7-difluoro-1-oxo-1,2-dihydroisoquinolin-4-yl) | |
| | ethyl)-N-methyl-1H-indole-2-carboxamide | |
| | enantiomer II | |
| 3 | 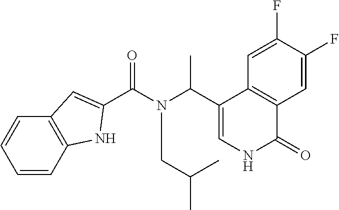 [SMILES: CC(C)CN(C(=O)c1cc2ccccc2[nH]1)C(C)c1c[nH]c(=O)c2cc(F)c(F)cc12] | 0.67 |
| | N-(1-(6,7-difluoro-1-oxo-1,2-dihydroisoquinolin-4-yl)ethyl)- | |
| | N-isobutyl-1H-indole-2-carboxamide | |
| | enantiomer I | |
| 4 | 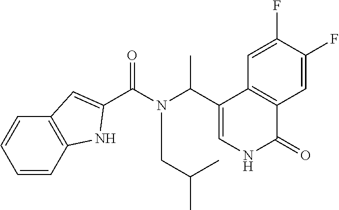 [SMILES: CC(C)CN(C(=O)c1cc2ccccc2[nH]1)C(C)c1c[nH]c(=O)c2cc(F)c(F)cc12] | 0.01 |
| | N-(1-(6,7-difluoro-1-oxo-1,2-dihydroisoquinolin-4-yl)ethyl)- | |
| | N-isobutyl-1H-indole-2-carboxamide | |
| | enantiomer II | |
| 5 | 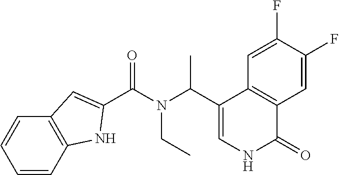 [SMILES: CCN(C(=O)c1cc2ccccc2[nH]1)C(C)c1c[nH]c(=O)c2cc(F)c(F)cc12] | 1.7 |
| | N-(1-(6,7-difluoro-1-oxo-1,2-dihydroisoquinolin-4-yl)ethyl)- | |
| | N-ethyl-1H-indole-2-carboxamide | |
| | enantiomer I | |
| 6 | 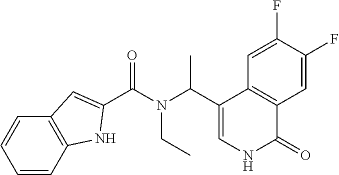 [SMILES: CCN(C(=O)c1cc2ccccc2[nH]1)C(C)c1c[nH]c(=O)c2cc(F)c(F)cc12] | 0.01 |
| | N-(1-(6,7-difluoro-1-oxo-1,2-dihydroisoquinolin-4-yl)ethyl)- | |
| | N-ethyl-1H-indole-2-carboxamide | |
| | enantiomer II | |
| 7 | 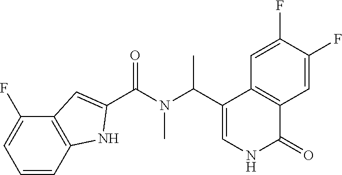 [SMILES: CC(c1c[nH]c(=O)c2cc(F)c(F)cc12)N(C)C(=O)c1cc2c(F)cccc2[nH]1] | 1.7 |
| | N-(1-(6,7-difluoro-1-oxo-1,2-dihydroisoquinolin-4-yl)ethyl)- | |
| | 4-fluoro-N-methyl-1H-indole-2-carboxamide | |
| | enantiomer I | |
| 8 | 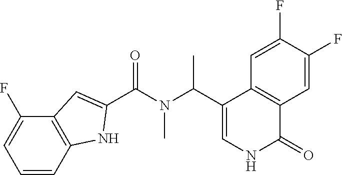 [SMILES: CC(c1c[nH]c(=O)c2cc(F)c(F)cc12)N(C)C(=O)c1cc2c(F)cccc2[nH]1] | 0.02 |
| | N-(1-(6,7-difluoro-1-oxo-1,2-dihydroisoquinolin-4-yl)ethyl)- | |
| | 4-fluoro-N-methyl-1H-indole-2-carboxamide | |
| | enantiomer II | |
| 9 | 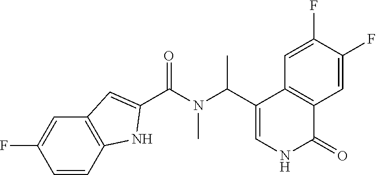 [SMILES: CC(c1c[nH]c(=O)c2cc(F)c(F)cc12)N(C)C(=O)c1cc2cc(F)ccc2[nH]1] | 1.6 |
| | N-(1-(6,7-difluoro-1-oxo-1,2-dihydroisoquinolin-4-yl)ethyl)- | |
| | 5-fluoro-N-methyl-1H-indole-2-carboxamide | |
| | enantiomer I | |
| 10 | 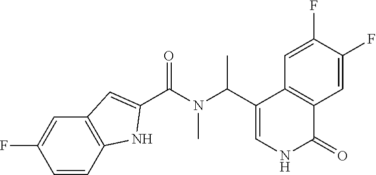 [SMILES: CC(c1c[nH]c(=O)c2cc(F)c(F)cc12)N(C)C(=O)c1cc2cc(F)ccc2[nH]1] | 0.02 |
| | N-(1-(6,7-difluoro-1-oxo-1,2-dihydroisoquinolin-4-yl)ethyl)- | |
| | 5-fluoro-N-methyl-1H-indole-2-carboxamide | |
| | enantiomer II | |
| 11 | 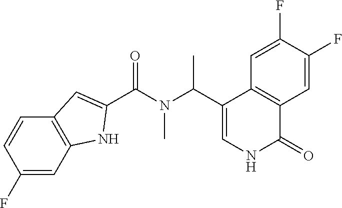 [SMILES: CC(c1c[nH]c(=O)c2cc(F)c(F)cc12)N(C)C(=O)c1cc2ccc(F)cc2[nH]1] | 1.2 |
| | N-(1-(6,7-difluoro-1-oxo-1,2-dihydroisoquinolin-4-yl)ethyl)- | |
| | 6-fluoro-N-methyl-1H-indole-2-carboxamide | |
| | enantiomer I | |
| 12 | 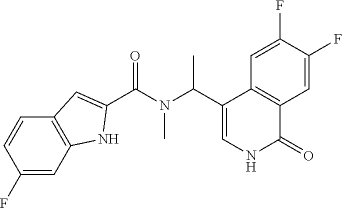 [SMILES: CC(c1c[nH]c(=O)c2cc(F)c(F)cc12)N(C)C(=O)c1cc2ccc(F)cc2[nH]1] | 0.02 |
| | N-(1-(6,7-difluoro-1-oxo-1,2-dihydroisoquinolin-4-yl)ethyl)- | |
| | 6-fluoro-N-methyl-1H-indole-2-carboxamide | |
| | enantiomer II | |
| 13 | 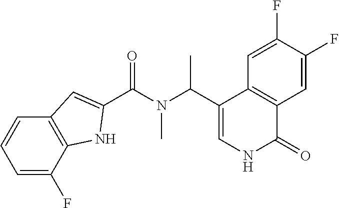 [SMILES: CC(c1c[nH]c(=O)c2cc(F)c(F)cc12)N(C)C(=O)c1cc2cccc(F)c2[nH]1] | 3.4 |
| | N-(1-(6,7-difluoro-1-oxo-1,2-dihydroisoquinolin-4-yl)ethyl)- | |
| | 7-fluoro-N-methyl-1H-indole-2-carboxamide | |
| | enantiomer I | |
| 14 | 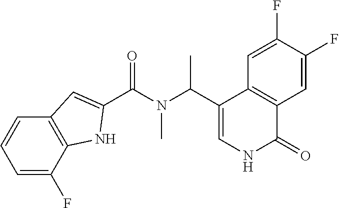 [SMILES: CC(c1c[nH]c(=O)c2cc(F)c(F)cc12)N(C)C(=O)c1cc2cccc(F)c2[nH]1] | 0.02 |
| | N-(1-(6,7-difluoro-1-oxo-1,2-dihydroisoquinolin-4-yl)ethyl)- | |
| | 7-fluoro-N-methyl-1H-indole-2-carboxamide | |
| | enantiomer II | |
| 15 | 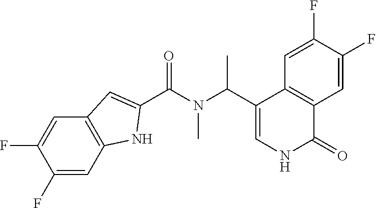 [SMILES: CC(c1c[nH]c(=O)c2cc(F)c(F)cc12)N(C)C(=O)c1cc2cc(F)c(F)cc2[nH]1] | 1.6 |
| | N-(1-(6,7-difluoro-1-oxo-1,2-dihydroisoquinolin-4-yl)ethyl)- | |
| | 5,6-fluoro-N-methyl-1H-indole-2-carboxamide | |
| | enantiomer I | |
| 16 | 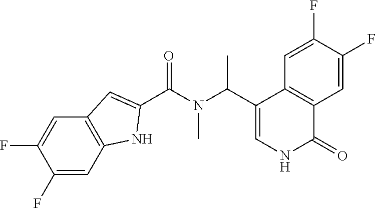 [SMILES: CC(c1c[nH]c(=O)c2cc(F)c(F)cc12)N(C)C(=O)c1cc2cc(F)c(F)cc2[nH]1] | 0.01 |
| | N-(1-(6,7-difluoro-1-oxo-1,2-dihydroisoquinolin-4-yl)ethyl)- | |
| | 5,6-fluoro-N-methyl-1H-indole-2-carboxamide | |
| | enantiomer II | |
| 17 | 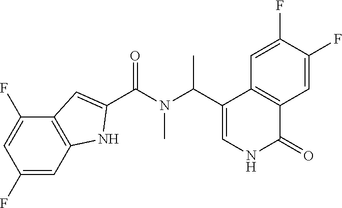 [SMILES: CC(c1c[nH]c(=O)c2cc(F)c(F)cc12)N(C)C(=O)c1cc2c(F)cc(F)cc2[nH]1] | 2.5 |
| | N-(1-(6,7-difluoro-1-oxo-1,2-dihydroisoquinolin-4-yl)ethyl)- | |
| | 4,6-difluoro-N-methyl-1H-indole-2-carboxamide | |
| | enantiomer I | |
| 18 | 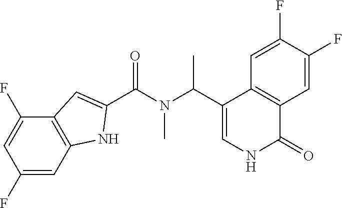 [SMILES: CC(c1c[nH]c(=O)c2cc(F)c(F)cc12)N(C)C(=O)c1cc2c(F)cc(F)cc2[nH]1] | 0.02 |
| | N-(1-(6,7-difluoro-1-oxo-1,2-dihydroisoquinolin-4-yl)ethyl)- | |
| | 4,6-difluoro-N-methyl-1H-indole-2-carboxamide | |
| | enantiomer II | |
| 19 | 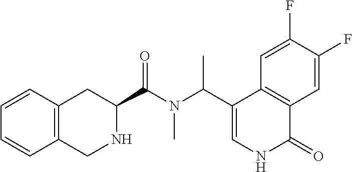 [SMILES: CC(c1c[nH]c(=O)c2cc(F)c(F)cc12)N(C)C(=O)[C@@H]1Cc2ccccc2CN1] | 0.82 |
| | (3S)-N-(1-(6,7-difluoro-1-oxo-1,2-dihydroisoquinolin-4-yl)ethyl)-N-methyl- | |
| | 1,2,3,4-tetrahydroisoquinoline-3-carboxamide | |
| | diastereoisomer I | |
| 20 | 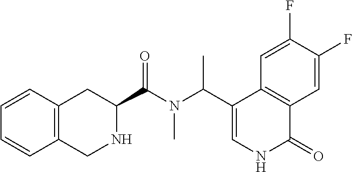 [SMILES: CC(c1c[nH]c(=O)c2cc(F)c(F)cc12)N(C)C(=O)[C@@H]1Cc2ccccc2CN1] | 9.2 |
| | (3S)-N-(1-(6,7-difluoro-1-oxo-1,2-dihydroisoquinolin-4-yl)ethyl)-N-methyl- | |
| | 1,2,3,4-tetrahydroisoquinoline-3-carboxamide | |
| | diastereoisomer II | |
| 21 | 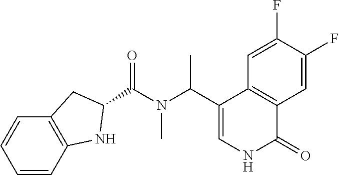 [SMILES: CC(c1c[nH]c(=O)c2cc(F)c(F)cc12)N(C)C(=O)[C@H]1Cc2ccccc2N1] | 0.30 |
| | (2R)-N-(1-(6,7-difluoro-1-oxo-1,2-dihydroisoquinolin-4-yl)ethyl)-N- | |
| | methylindoline-2-carboxamide | |
| | diastereoisomer I | |
| 22 | 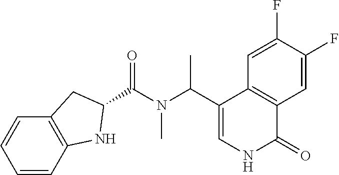 [SMILES: CC(c1c[nH]c(=O)c2cc(F)c(F)cc12)N(C)C(=O)[C@H]1Cc2ccccc2N1] | 3.7 |
| | (2R)-N-(1-(6,7-difluoro-1-oxo-1,2-dihydroisoquinolin-4-yl)ethyl)-N- | |
| | methylindoline-2-carboxamide | |
| | diastereoisomer II | |
| 23 | 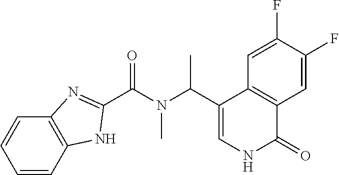 [SMILES: CC(c1c[nH]c(=O)c2cc(F)c(F)cc12)N(C)C(=O)c1nc2ccccc2[nH]1] | 25 |
| | N-(1-(6,7-difluoro-1-oxo-1,2-dihydroisoquinolin-4-yl)ethyl)-N-methyl-1H- | |
| | benzo[d]imidazole-2-carboxamide | |
| | enantiomer I | |
| 24 | 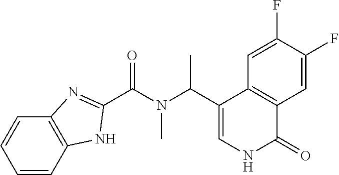 [SMILES: CC(c1c[nH]c(=O)c2cc(F)c(F)cc12)N(C)C(=O)c1nc2ccccc2[nH]1] | 0.76 |
| | N-(1-(6,7-difluoro-1-oxo-1,2-dihydroisoquinolin-4-yl)ethyl)-N-methyl-1H- | |
| | benzo[d]imidazole-2-carboxamide | |
| | enantiomer II | |
| 25 | 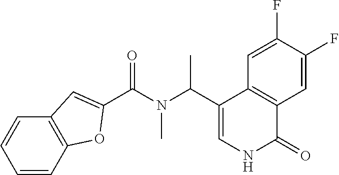 [SMILES: CC(c1c[nH]c(=O)c2cc(F)c(F)cc12)N(C)C(=O)c1cc2ccccc2o1] | 17 |
| | N-(1-(6,7-difluoro-1-oxo-1,2-dihydroisoquinolin-4-yl)ethyl)-N- | |
| | methylbenzofuran-2-carboxamide | |
| | enantiomer I | |
| 26 | 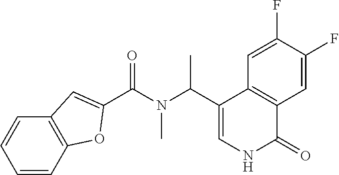 [SMILES: CC(c1c[nH]c(=O)c2cc(F)c(F)cc12)N(C)C(=O)c1cc2ccccc2o1] | 0.35 |
| | N-(1-(6,7-difluoro-1-oxo-1,2-dihydroisoquinolin-4-yl)ethyl)-N- | |
| | methylbenzofuran-2-carboxamide | |
| | enantiomer II | |
| 27 | 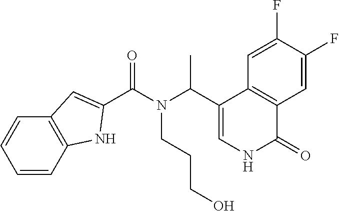 [SMILES: CC(c1c[nH]c(=O)c2cc(F)c(F)cc12)N(CCCO)C(=O)c1cc2ccccc2[nH]1] | 2.9 |
| | N-(1-(6,7-difluoro-1-oxo-1,2-dihydroisoquinolin-4-yl)ethyl)-N-(3- | |
| | hydroxypropyl)-1H-indole-2-carboxamide | |
| | enantiomer I | |
| 28 | 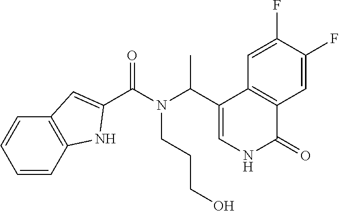 [SMILES: CC(c1c[nH]c(=O)c2cc(F)c(F)cc12)N(CCCO)C(=O)c1cc2ccccc2[nH]1] | 0.04 |
| | N-(1-(6,7-difluoro-1-oxo-1,2-dihydroisoquinolin-4-yl)ethyl)-N-(3- | |
| | hydroxypropyl)-1H-indole-2-carboxamide | |
| | enantiomer II | |
| 29 | 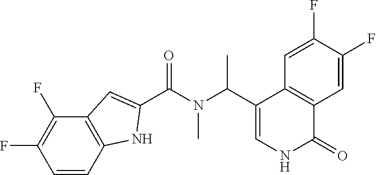 [SMILES: CC(c1c[nH]c(=O)c2cc(F)c(F)cc12)N(C)C(=O)c1cc2c(F)c(F)ccc2[nH]1] | 0.04 |
| | N-(1-(6,7-difluoro-1-oxo-1,2-dihydroisoquinolin-4-yl)ethyl)-4,5-difluoro-N- | |
| | methyl-1H-indole-2-carboxamide | |
| 30 | 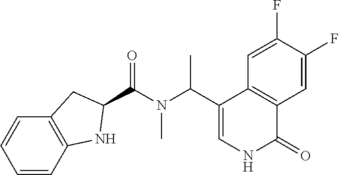 [SMILES: CC(c1c[nH]c(=O)c2cc(F)c(F)cc12)N(C)C(=O)[C@@H]1Cc2ccccc2N1] | 0.02 |
| | (2S)-N-(1-(6,7-difluoro-1-oxo-1,2-dihydroisoquinolin-4-yl)ethyl)-N- | |
| | methylindoline-2-carboxamide | |
| | diastereoisomer I | |
| 31 | 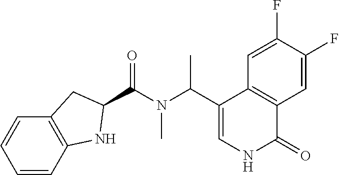 [SMILES: CC(c1c[nH]c(=O)c2cc(F)c(F)cc12)N(C)C(=O)[C@@H]1Cc2ccccc2N1] | 20 |
| | (2S)-N-(1-(6,7-difluoro-1-oxo-1,2-dihydroisoquinolin-4-yl)ethyl)-N- | |
| | methylindoline-2-carboxamide | |
| | diastereoisomer II | |
| 32 | 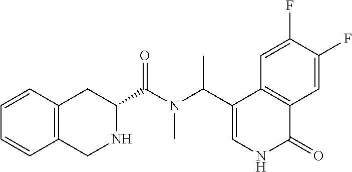 [SMILES: CC(c1c[nH]c(=O)c2cc(F)c(F)cc12)N(C)C(=O)[C@H]1Cc2ccccc2CN1] | 25 |
| | (3R)-N-(1-(6,7-difluoro-1-oxo-1,2-dihydroisoquinolin-4-yl)ethyl)-N-methyl- | |
| | 1,2,3,4-tetrahydroisoquinoline-3-carboxamide | |
| | diastereoisomer I | |
| 33 | 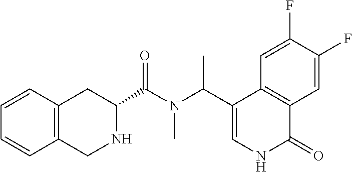 [SMILES: CC(c1c[nH]c(=O)c2cc(F)c(F)cc12)N(C)C(=O)[C@H]1Cc2ccccc2CN1] | 6.6 |
| | (3R)-N-(1-(6,7-difluoro-1-oxo-1,2-dihydroisoquinolin-4-yl)ethyl)-N-methyl- | |
| | 1,2,3,4-tetrahydroisoquinoline-3-carboxamide | |
| | diastereoisomer II | |
| 34 | 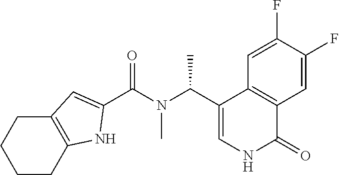 [SMILES: C[C@H](c1c[nH]c(=O)c2cc(F)c(F)cc12)N(C)C(=O)c1cc2c([nH]1)CCCC2] | 0.05 |
| | (R)-N-(1-(6,7-difluoro-1-oxo-1,2-dihydroisoquinolin-4-yl)ethyl)-N-methyl- | |
| | 4,5,6,7-tetrahydro-1H-indole-2-carboxamide | |
| | single enantiomer | |
| 43 | 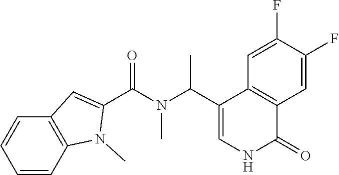 [SMILES: CC(c1c[nH]c(=O)c2cc(F)c(F)cc12)N(C)C(=O)c1cc2ccccc2n1C] | 4.3 |
| | N-(1-(6,7-difluoro-1-oxo-1,2-dihydroisoquinolin-4-yl)ethyl)-N,1-dimethyl- | |
| | 1H-indole-2-carboxamide | |
| | enantiomer I | |
| 44 | 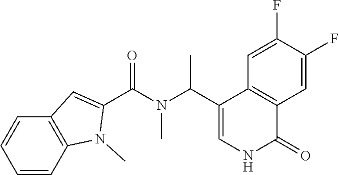 [SMILES: CC(c1c[nH]c(=O)c2cc(F)c(F)cc12)N(C)C(=O)c1cc2ccccc2n1C] | 0.30 |
| | N-(1-(6,7-difluoro-1-oxo-1,2-dihydroisoquinolin-4-yl)ethyl)-N,1-dimethyl- | |
| | 1H-indole-2-carboxamide | |
| | enantiomer II | |
| 45 | 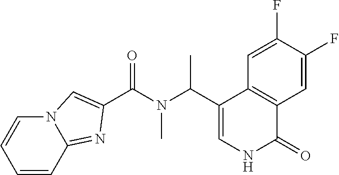 [SMILES: CC(c1c[nH]c(=O)c2cc(F)c(F)cc12)N(C)C(=O)c1cn2ccccc2n1] | 2.5 |
| | N-(1-(6,7-difluoro-1-oxo-1,2-dihydroisoquinolin-4-yl)ethyl)-N- | |
| | methylimidazo[1,2-a]pyridine-2-carboxamide | |
| | enantiomer I | |
| 46 | 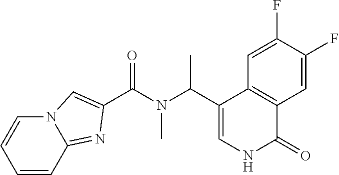 [SMILES: CC(c1c[nH]c(=O)c2cc(F)c(F)cc12)N(C)C(=O)c1cn2ccccc2n1] | 21 |
| | N-(1-(6,7-difluoro-1-oxo-1,2-dihydroisoquinolin-4-yl)ethyl)-N- | |
| | methylimidazo[1,2-a]pyridine-2-carboxamide | |
| | enantiomer II | |
| 47 | 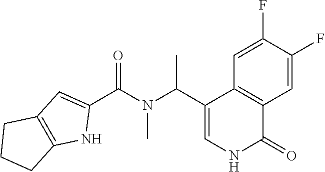 [SMILES: CC(c1c[nH]c(=O)c2cc(F)c(F)cc12)N(C)C(=O)c1cc2c([nH]1)CCC2] | 25 |
| | N-(1-(6,7-difluoro-1-oxo-1,2-dihydroisoquinolin-4-yl)ethyl)-N-methyl- | |
| | 1,4,5,6-tetrahydrocyclopenta[b]pyrrole-2-carboxamide | |
| | enantiomer I | |
| 48 | 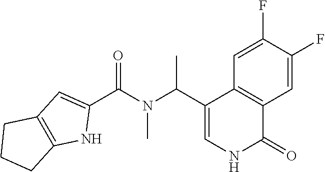 [SMILES: CC(c1c[nH]c(=O)c2cc(F)c(F)cc12)N(C)C(=O)c1cc2c([nH]1)CCC2] | 0.15 |
| | N-(1-(6,7-difluoro-1-oxo-1,2-dihydroisoquinolin-4-yl)ethyl)-N-methyl- | |
| | 1,4,5,6-tetrahydrocyclopenta[b]pyrrole-2-carboxamide | |
| | enantiomer II | |
| 49 | 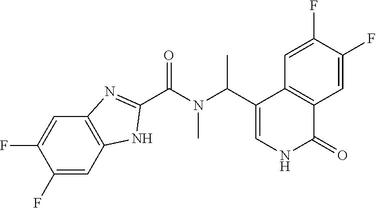 [SMILES: CC(c1c[nH]c(=O)c2cc(F)c(F)cc12)N(C)C(=O)c1nc2cc(F)c(F)cc2[nH]1] | 3.1 |
| | N-(1-(6,7-difluoro-1-oxo-1,2-dihydroisoquinolin-4-yl)ethyl)-5,6-difluoro-N- | |
| | methyl-1H-benzo[d]imidazole-2-carboxamide | |
| | enantiomer I | |
| 50 | 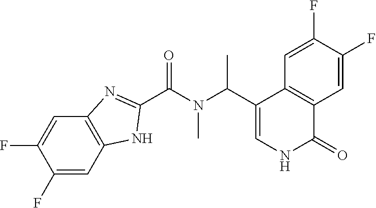 [SMILES: CC(c1c[nH]c(=O)c2cc(F)c(F)cc12)N(C)C(=O)c1nc2cc(F)c(F)cc2[nH]1] | 0.29 |
| | N-(1-(6,7-difluoro-1-oxo-1,2-dihydroisoquinolin-4-yl)ethyl)-5,6-difluoro-N- | |
| | methyl-1H-benzo[d]imidazole-2-carboxamide | |
| | enantiomer II | |
| 51 | 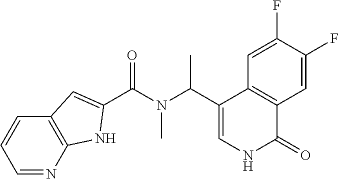 [SMILES: CC(c1c[nH]c(=O)c2cc(F)c(F)cc12)N(C)C(=O)c1cc2cccnc2[nH]1] | 0.79 |
| | N-(1-(6,7-difluoro-1-oxo-1,2-dihydroisoquinolin-4-yl)ethyl)-N-methyl-1H- | |
| | pyrrolo[2,3-b]pyridine-2-carboxamide | |
| | enantiomer I | |
| 52 | 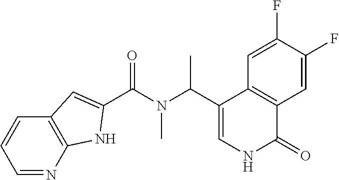 [SMILES: CC(c1c[nH]c(=O)c2cc(F)c(F)cc12)N(C)C(=O)c1cc2cccnc2[nH]1] | 25 |
| | N-(1-(6,7-difluoro-1-oxo-1,2-dihydroisoquinolin-4-yl)ethyl)-N-methyl-1H- | |
| | pyrrolo[2,3-b]pyridine-2-carboxamide | |
| | enantiomer II | |
| 53 | 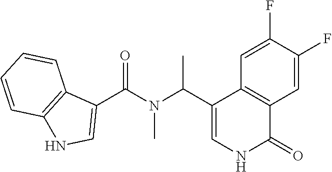 [SMILES: CC(c1c[nH]c(=O)c2cc(F)c(F)cc12)N(C)C(=O)c1c[nH]c2ccccc12] | 25 |
| | N-(1-(6,7-difluoro-1-oxo-1,2-dihydroisoquinolin-4-yl)ethyl)-N-methyl-1H- | |
| | indole-3-carboxamide | |
| | enantiomer I | |
| 54 | 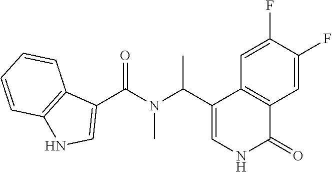 [SMILES: CC(c1c[nH]c(=O)c2cc(F)c(F)cc12)N(C)C(=O)c1c[nH]c2ccccc12] | 5.1 |
| | N-(1-(6,7-difluoro-1-oxo-1,2-dihydroisoquinolin-4-yl)ethyl)-N-methyl-1H- | |
| | indole-3-carboxamide | |
| | enantiomer II | |
| 55 | 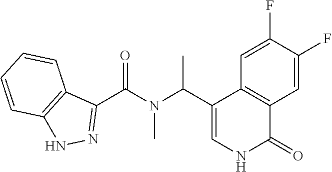 [SMILES: CC(c1c[nH]c(=O)c2cc(F)c(F)cc12)N(C)C(=O)c1n[nH]c2ccccc12] | 16 |
| | N-(1-(6,7-difluoro-1-oxo-1,2-dihydroisoquinolin-4-yl)ethyl)-N-methyl-1H- | |
| | indazole-3-carboxamide | |
| | enantiomer I | |
| 56 | 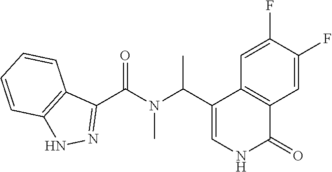 [SMILES: CC(c1c[nH]c(=O)c2cc(F)c(F)cc12)N(C)C(=O)c1n[nH]c2ccccc12] | 25 |
| | N-(1-(6,7-difluoro-1-oxo-1,2-dihydroisoquinolin-4-yl)ethyl)-N-methyl-1H- | |
| | indazole-3-carboxamide | |
| | enantiomer II | |
| 57 | 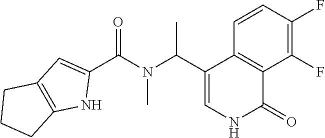 [SMILES: CC(c1c[nH]c(=O)c2c(F)c(F)ccc12)N(C)C(=O)c1cc2c([nH]1)CCC2] | 12 |
| | N-(1-(7,8-difluoro-1-oxo-1,2-dihydroisoquinolin-4-yl)ethyl)-N-methyl- | |
| | 1,4,5,6-tetrahydrocyclopenta[b]pyrrole-2-carboxamide | |
| | enantiomer I | |
| 58 | 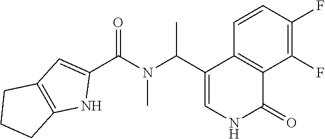 [SMILES: CC(c1c[nH]c(=O)c2c(F)c(F)ccc12)N(C)C(=O)c1cc2c([nH]1)CCC2] | 1.8 |
| | N-(1-(7,8-difluoro-1-oxo-1,2-dihydroisoquinolin-4-yl)ethyl)-N-methyl- | |
| | 1,4,5,6-tetrahydrocyclopenta[b]pyrrole-2-carboxamide | |
| | enantiomer II | |
| 59 | 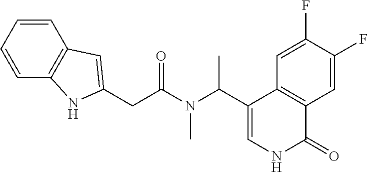 [SMILES: CC(c1c[nH]c(=O)c2cc(F)c(F)cc12)N(C)C(=O)Cc1cc2ccccc2[nH]1] | 0.91 |
| | N-(1-(6,7-difluoro-1-oxo-1,2-dihydroisoquinolin-4-yl)ethyl)-2-(1H-indol-2- | |
| | yl)-N-methylacetamide | |
| 60 | 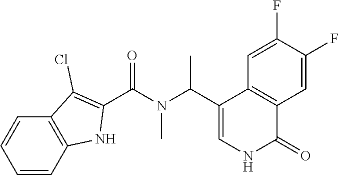 [SMILES: CC(c1c[nH]c(=O)c2cc(F)c(F)cc12)N(C)C(=O)c1[nH]c2ccccc2c1Cl] | 0.80 |
| | 3-chloro-N-(1-(6,7-difluoro-1-oxo-1,2-dihydroisoquinolin-4-yl)ethyl)-N- | |
| | methyl-1H-indole-2-carboxamide | |
| 61 | 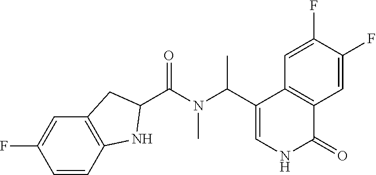 [SMILES: CC(c1c[nH]c(=O)c2cc(F)c(F)cc12)N(C)C(=O)C1Cc2cc(F)ccc2N1] | 6.7 |
| | N-(1-(6,7-difluoro-1-oxo-1,2-dihydroisoquinolin-4-yl)ethyl)-5-fluoro-N- | |
| | methylindoline-2-carboxamide | |
| | stereoisomer IA | |
| 62 | 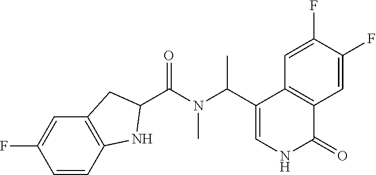 [SMILES: CC(c1c[nH]c(=O)c2cc(F)c(F)cc12)N(C)C(=O)C1Cc2cc(F)ccc2N1] | 0.19 |
| | N-(1-(6,7-difluoro-1-oxo-1,2-dihydroisoquinolin-4-yl)ethyl)-5-fluoro-N- | |
| | methylindoline-2-carboxamide | |
| | stereoisomer IIA | |
| 63 | 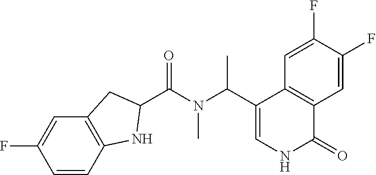 [SMILES: CC(c1c[nH]c(=O)c2cc(F)c(F)cc12)N(C)C(=O)C1Cc2cc(F)ccc2N1] | 5.2 |
| | N-(1-(6,7-difluoro-1-oxo-1,2-dihydroisoquinolin-4-yl)ethyl)-5-fluoro-N- | |
| | methylindoline-2-carboxamide | |
| | stereoisomer IB | |
| 64 | 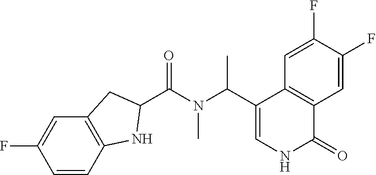 [SMILES: CC(c1c[nH]c(=O)c2cc(F)c(F)cc12)N(C)C(=O)C1Cc2cc(F)ccc2N1] | 0.02 |
| | N-(1-(6,7-difluoro-1-oxo-1,2-dihydroisoquinolin-4-yl)ethyl)-5-fluoro-N- | |
| | methylindoline-2-carboxamide | |
| | stereoisomer IIB | |
| 65 | 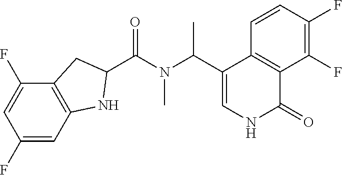 [SMILES: CC(c1c[nH]c(=O)c2c(F)c(F)ccc12)N(C)C(=O)C1Cc2c(F)cc(F)cc2N1] | 11 |
| | N-(1-(7,8-difluoro-1-oxo-1,2-dihydroisoquinolin-4-yl)ethyl)-4,6-difluoro-N- | |
| | methylindoline-2-carboxamide | |
| | stereoisomer IIA | |
| 66 | 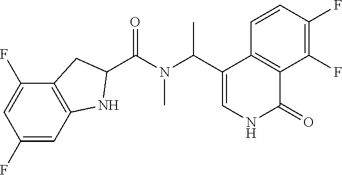 [SMILES: CC(c1c[nH]c(=O)c2c(F)c(F)ccc12)N(C)C(=O)C1Cc2c(F)cc(F)cc2N1] | 0.21 |
| | N-(1-(7,8-difluoro-1-oxo-1,2-dihydroisoquinolin-4-yl)ethyl)-4,6-difluoro-N- | |
| | methylindoline-2-carboxamide | |
| | stereoisomer IA | |
| 67 | 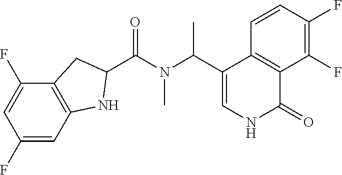 [SMILES: CC(c1c[nH]c(=O)c2c(F)c(F)ccc12)N(C)C(=O)C1Cc2c(F)cc(F)cc2N1] | 3.1 |
| | N-(1-(7,8-difluoro-1-oxo-1,2-dihydroisoquinolin-4-yl)ethyl)-4,6-difluoro-N- | |
| | methylindoline-2-carboxamide | |
| | stereoisomer IIB | |
| 68 | 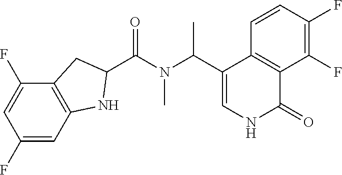 [SMILES: CC(c1c[nH]c(=O)c2c(F)c(F)ccc12)N(C)C(=O)C1Cc2c(F)cc(F)cc2N1] | 0.20 |
| | N-(1-(7,8-difluoro-1-oxo-1,2-dihydroisoquinolin-4-yl)ethyl)-4,6-difluoro-N- | |
| | methylindoline-2-carboxamide | |
| | stereoisomer IB | |
| 69 | 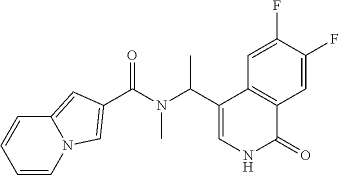 [SMILES: CC(c1c[nH]c(=O)c2cc(F)c(F)cc12)N(C)C(=O)c1cc2ccccn2c1] | 5.7 |
| | N-(1-(6,7-difluoro-1-oxo-1,2-dihydroisoquinolin-4-yl)ethyl)-N- | |
| | methylindolizine-2-carboxamide | |
| | enantiomer I | |
| 70 | 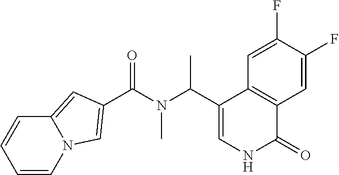 [SMILES: CC(c1c[nH]c(=O)c2cc(F)c(F)cc12)N(C)C(=O)c1cc2ccccn2c1] | 0.02 |
| | N-(1-(6,7-difluoro-1-oxo-1,2-dihydroisoquinolin-4-yl)ethyl)-N- | |
| | methylindolizine-2-carboxamide | |
| | enantiomer II | |
| 71 | 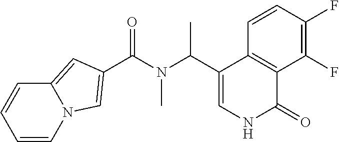 [SMILES: CC(c1c[nH]c(=O)c2c(F)c(F)ccc12)N(C)C(=O)c1cc2ccccn2c1] | 6.5 |
| | N-(1-(7,8-difluoro-1-oxo-1,2-dihydroisoquinolin-4-yl)ethyl)-N- | |
| | methylindolizine-2-carboxamide | |
| | enantiomer I | |
| 72 | 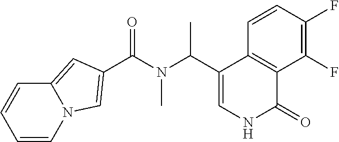 [SMILES: CC(c1c[nH]c(=O)c2c(F)c(F)ccc12)N(C)C(=O)c1cc2ccccn2c1] | 0.14 |
| | N-(1-(7,8-difluoro-1-oxo-1,2-dihydroisoquinolin-4-yl)ethyl)-N- | |
| | methylindolizine-2-carboxamide | |
| | enantiomer II | |
| 73 | 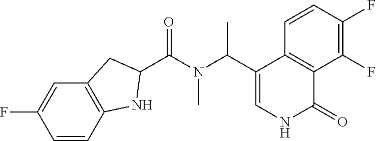 [SMILES: CC(c1c[nH]c(=O)c2c(F)c(F)ccc12)N(C)C(=O)C1Cc2cc(F)ccc2N1] | 10 |
| | N-(1-(7,8-difluoro-1-oxo-1,2-dihydroisoquinolin-4-yl)ethyl)-5-fluoro-N- | |
| | methylindoline-2-carboxamide | |
| | stereoisomer IIA | |
| 74 | 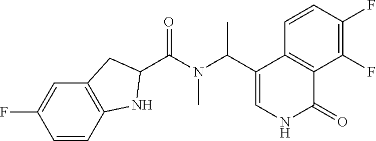 [SMILES: CC(c1c[nH]c(=O)c2c(F)c(F)ccc12)N(C)C(=O)C1Cc2cc(F)ccc2N1] | 0.26 |
| | N-(1-(7,8-difluoro-1-oxo-1,2-dihydroisoquinolin-4-yl)ethyl)-5-fluoro-N- | |
| | methylindoline-2-carboxamide | |
| | stereoisomer IA | |
| 75 | 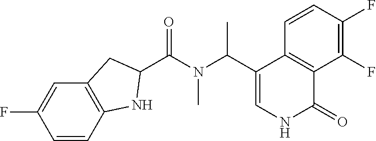 [SMILES: CC(c1c[nH]c(=O)c2c(F)c(F)ccc12)N(C)C(=O)C1Cc2cc(F)ccc2N1] | 0.83 |
| | N-(1-(7,8-difluoro-1-oxo-1,2-dihydroisoquinolin-4-yl)ethyl)-5-fluoro-N- | |
| | methylindoline-2-carboxamide | |
| | stereoisomer IIB | |
| 76 | 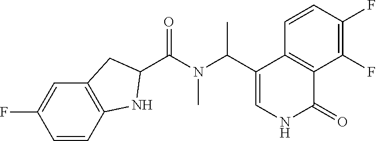 [SMILES: CC(c1c[nH]c(=O)c2c(F)c(F)ccc12)N(C)C(=O)C1Cc2cc(F)ccc2N1] | 4.4 |
| | N-(1-(7,8-difluoro-1-oxo-1,2-dihydroisoquinolin-4-yl)ethyl)-5-fluoro-N- | |
| | methylindoline-2-carboxamide | |
| | stereoisomer IB | |
| 77 | 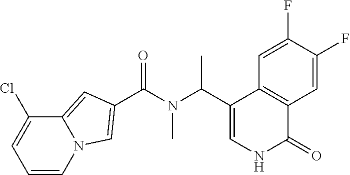 [SMILES: CC(c1c[nH]c(=O)c2cc(F)c(F)cc12)N(C)C(=O)c1cc2c(Cl)cccn2c1] | 2.0 |
| | 8-chloro-N-(1-(6,7-difluoro-1-oxo-1,2-dihydroisoquinolin-4-yl)ethyl)-N- | |
| | methylindolizine-2-carboxamide | |
| | enantiomer I | |
| 78 | 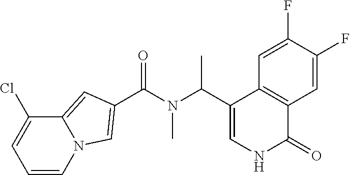 [SMILES: CC(c1c[nH]c(=O)c2cc(F)c(F)cc12)N(C)C(=O)c1cc2c(Cl)cccn2c1] | 0.01 |
| | 8-chloro-N-(1-(6,7-difluoro-1-oxo-1,2-dihydroisoquinolin-4-yl)ethyl)-N- | |
| | methylindolizine-2-carboxamide | |
| | enantiomer II | |
| 79 | 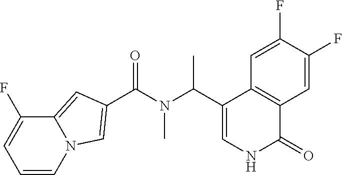 [SMILES: CC(c1c[nH]c(=O)c2cc(F)c(F)cc12)N(C)C(=O)c1cc2c(F)cccn2c1] | 3.3 |
| | N-(1-(6,7-difluoro-1-oxo-1,2-dihydroisoquinolin-4-yl)ethyl)-8-fluoro-N- | |
| | methylindolizine-2-carboxamide | |
| | enantiomer I | |
| 80 | 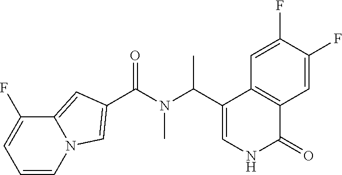 [SMILES: CC(c1c[nH]c(=O)c2cc(F)c(F)cc12)N(C)C(=O)c1cc2c(F)cccn2c1] | 0.02 |
| | N-(1-(6,7-difluoro-1-oxo-1,2-dihydroisoquinolin-4-yl)ethyl)-8-fluoro-N- | |
| | methylindolizine-2-carboxamide | |
| | enantiomer II | |
| 81 | 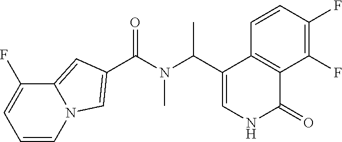 [SMILES: CC(c1c[nH]c(=O)c2c(F)c(F)ccc12)N(C)C(=O)c1cc2c(F)cccn2c1] | 2.1 |
| | N-(1-(7,8-difluoro-1-oxo-1,2-dihydroisoquinolin-4-yl)ethyl)-8-fluoro-N- | |
| | methylindolizine-2-carboxamide | |
| | enantiomer I | |
| 82 | 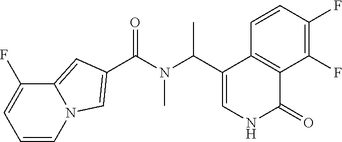 [SMILES: CC(c1c[nH]c(=O)c2c(F)c(F)ccc12)N(C)C(=O)c1cc2c(F)cccn2c1] | 0.07 |
| | N-(1-(7,8-difluoro-1-oxo-1,2-dihydroisoquinolin-4-yl)ethyl)-8-fluoro-N- | |
| | methylindolizine-2-carboxamide | |
| | enantiomer II | |
| 83 | 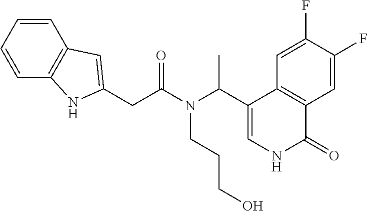 [SMILES: CC(c1c[nH]c(=O)c2cc(F)c(F)cc12)N(CCCO)C(=O)Cc1cc2ccccc2[nH]1] | 25 |
| | N-(1-(6,7-difluoro-1-oxo-1,2-dihydroisoquinolin-4-yl)ethyl)-N-(3- | |
| | hydroxypropyl)-2-(1H-indol-2-yl)acetamide | |
| | enantiomer I | |
| 84 | 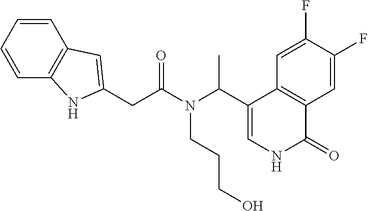 [SMILES: CC(c1c[nH]c(=O)c2cc(F)c(F)cc12)N(CCCO)C(=O)Cc1cc2ccccc2[nH]1] | 0.42 |
| | N-(1-(6,7-difluoro-1-oxo-1,2-dihydroisoquinolin-4-yl)ethyl)-N-(3- | |
| | hydroxypropyl)-2-(1H-indol-2-yl)acetamide | |
| | enantiomer II | |
| 87 | 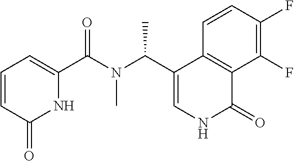 [SMILES: C[C@H](c1c[nH]c(=O)c2c(F)c(F)ccc12)N(C)C(=O)c1cccc(=O)[nH]1] | 18 |
| | (R)-N-(1-(7,8-difluoro-1-oxo-1,2-dihydroisoquinolin-4-yl)ethyl)- | |
| | N-methyl-6-oxo-1,6-dihydropyridine-2-carboxamide | |
| 88 | 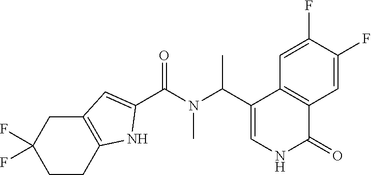 [SMILES: CC(c1c[nH]c(=O)c2cc(F)c(F)cc12)N(C)C(=O)c1cc2c([nH]1)CCC(F)(F)C2] | 0.03 |
| | N-(1-(6,7-difluoro-1-oxo-1,2-dihydroisoquinolin-4-yl)ethyl)-5,5- | |
| | difluoro-N-methyl-4,5,6,7-tetrahydro-1H-indole-2-carboxamide | |
| | enantiomer I | |
| 89 | 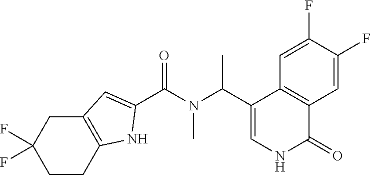 [SMILES: CC(c1c[nH]c(=O)c2cc(F)c(F)cc12)N(C)C(=O)c1cc2c([nH]1)CCC(F)(F)C2] | 1.8 |
| | N-(1-(6,7-difluoro-1-oxo-1,2-dihydroisoquinolin-4-yl)ethyl)-5,5-difluoro-N- | |
| | methyl-4,5,6,7-tetrahydro-1H-indole-2-carboxamide | |
| | enantiomer II | |
| 90 | 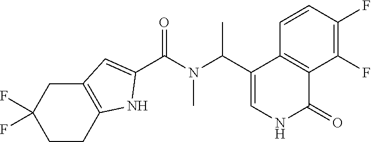 [SMILES: CC(c1c[nH]c(=O)c2c(F)c(F)ccc12)N(C)C(=O)c1cc2c([nH]1)CCC(F)(F)C2] | 0.37 |
| | N-(1-(7,8-difluoro-1-oxo-1,2-dihydroisoquinolin-4-yl)ethyl)-5,5-difluoro-N- | |
| | methyl-4,5,6,7-tetrahydro-1H-indole-2-carboxamide | |
| | enantiomer I | |
| 91 | 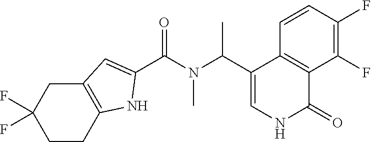 [SMILES: CC(c1c[nH]c(=O)c2c(F)c(F)ccc12)N(C)C(=O)c1cc2c([nH]1)CCC(F)(F)C2] | 7.3 |
| | N-(1-(7,8-difluoro-1-oxo-1,2-dihydroisoquinolin-4-yl)ethyl)-5,5-difluoro-N- | |
| | methyl-4,5,6,7-tetrahydro-1H-indole-2-carboxamide | |
| | enantiomer II | |
| 92 | 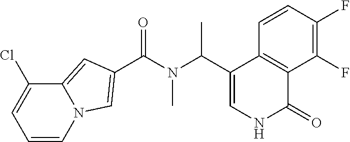 [SMILES: CC(c1c[nH]c(=O)c2c(F)c(F)ccc12)N(C)C(=O)c1cc2c(Cl)cccn2c1] | 2.7 |
| | 8-chloro-N-(1-(7,8-difluoro-1-oxo-1,2-dihydroisoquinolin-4-yl)ethyl)-N- | |
| | methylindolizine-2-carboxamide | |
| | enantiomer I | |
| 93 | 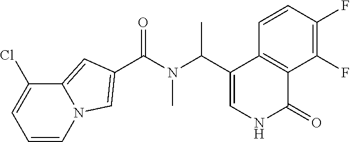 [SMILES: CC(c1c[nH]c(=O)c2c(F)c(F)ccc12)N(C)C(=O)c1cc2c(Cl)cccn2c1] | 0.04 |
| | 8-chloro-N-(1-(7,8-difluoro-1-oxo-1,2-dihydroisoquinolin-4-yl)ethyl)-N- | |
| | methylindolizine-2-carboxamide | |
| | enantiomer II | |
| 94 | 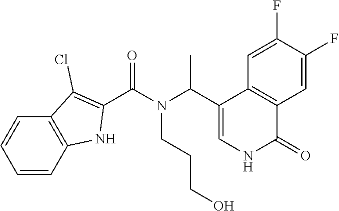 [SMILES: CC(c1c[nH]c(=O)c2cc(F)c(F)cc12)N(CCCO)C(=O)c1[nH]c2ccccc2c1Cl] | 19 |
| | 3-chloro-N-(1-(6,7-difluoro-1-oxo-1,2-dihydroisoquinolin-4-yl)ethyl)-N-(3- | |
| | hydroxypropyl)-1H-indole-2-carboxamide | |
| | enantiomer I | |
| 95 | 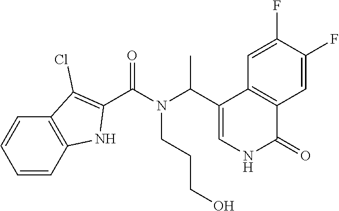 [SMILES: CC(c1c[nH]c(=O)c2cc(F)c(F)cc12)N(CCCO)C(=O)c1[nH]c2ccccc2c1Cl] | 0.40 |
| | 3-chloro-N-(1-(6,7-difluoro-1-oxo-1,2-dihydroisoquinolin-4-yl)ethyl)-N-(3- | |
| | hydroxypropyl)-1H-indole-2-carboxamide | |
| | enantiomer II | |
| 96 | 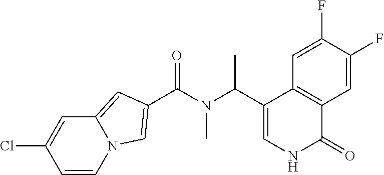 [SMILES: CC(c1c[nH]c(=O)c2cc(F)c(F)cc12)N(C)C(=O)c1cc2cc(Cl)ccn2c1] | 4.6 |
| | 7-chloro-N-(1-(6,7-difluoro-1-oxo-1,2-dihydroisoquinolin-4-yl)ethyl)-N- | |
| | methylindolizine-2-carboxamide | |
| | enantiomer I | |
| 97 | 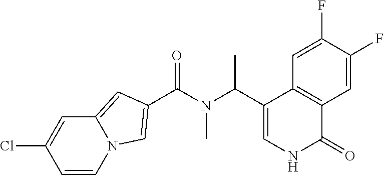 [SMILES: CC(c1c[nH]c(=O)c2cc(F)c(F)cc12)N(C)C(=O)c1cc2cc(Cl)ccn2c1] | 0.09 |
| | 7-chloro-N-(1-(6,7-difluoro-1-oxo-1,2-dihydroisoquinolin-4-yl)ethyl)-N- | |
| | methylindolizine-2-carboxamide | |
| | enantiomer II | |
| 98 | 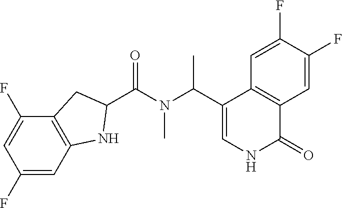 [SMILES: CC(c1c[nH]c(=O)c2cc(F)c(F)cc12)N(C)C(=O)C1Cc2c(F)cc(F)cc2N1] | 5.9 |
| | N-(1-(6,7-difluoro-1-oxo-1,2-dihydroisoquinolin-4-yl)ethy1)-4,6-difluoro-N- | |
| | methylindoline-2-carboxamide | |
| | stereoisomer IA | |
| 99 | 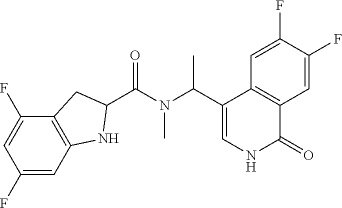 [SMILES: CC(c1c[nH]c(=O)c2cc(F)c(F)cc12)N(C)C(=O)C1Cc2c(F)cc(F)cc2N1] | 0.07 |
| | N-(1-(6,7-difluoro-1-oxo-1,2-dihydroisoquinolin-4-yl)ethyl)-4,6-difluoro-N- | |
| | methylindoline-2-carboxamide | |
| | stereoisomer IIA | |
| 100 | 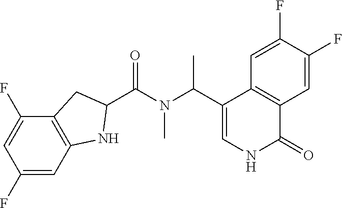 [SMILES: CC(c1c[nH]c(=O)c2cc(F)c(F)cc12)N(C)C(=O)C1Cc2c(F)cc(F)cc2N1] | 0.80 |
| | N-(1-(6,7-difluoro-1-oxo-1,2-dihydroisoquinolin-4-yl)ethyl)-4,6-difluoro-N- | |
| | methylindoline-2-carboxamide | |
| | stereoisomer IB | |
| 101 | 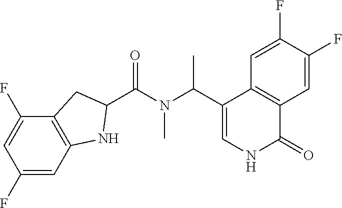 [SMILES: CC(c1c[nH]c(=O)c2cc(F)c(F)cc12)N(C)C(=O)C1Cc2c(F)cc(F)cc2N1] | 0.03 |
| | N-(1-(6,7-difluoro-1-oxo-1,2-dihydroisoquinolin-4-yl)ethyl)-4,6-difluoro-N- | |
| | methylindoline-2-carboxamide | |
| | stereoisomer IIB | |
| 102 | 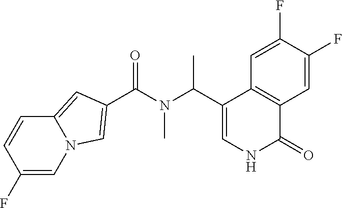 [SMILES: CC(c1c[nH]c(=O)c2cc(F)c(F)cc12)N(C)C(=O)c1cc2ccc(F)cn2c1] | 12 |
| | N-(1-(6,7-difluoro-1-oxo-1,2-dihydroisoquinolin-4-yl)ethyl)-6-fluoro-N- | |
| | methylindolizine-2-carboxamide | |
| | enantiomer I | |
| 103 | 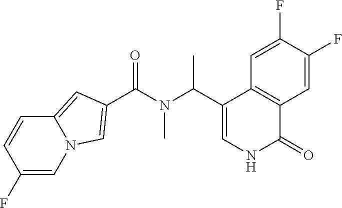 [SMILES: CC(c1c[nH]c(=O)c2cc(F)c(F)cc12)N(C)C(=O)c1cc2ccc(F)cn2c1] | 0.03 |
| | N-(1-(6,7-difluoro-1-oxo-1,2-dihydroisoquinolin-4-yl)ethyl)-6-fluoro-N- | |
| | methylindolizine-2-carboxamide | |
| | enantiomer II | |
| 104 | 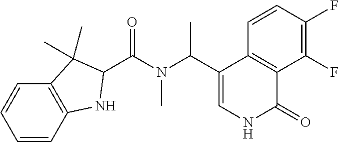 [SMILES: CC(c1c[nH]c(=O)c2c(F)c(F)ccc12)N(C)C(=O)C1Nc2ccccc2C1(C)C] | 25 |
| | N-(1-(7,8-difluoro-1-oxo-1,2-dihydroisoquinolin-4-yl)ethyl)-N,3,3- | |
| | trimethylindoline-2-carboxamide | |
| | stereoisomer IA | |
| 105 | 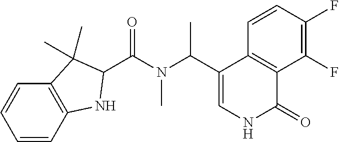 [SMILES: CC(c1c[nH]c(=O)c2c(F)c(F)ccc12)N(C)C(=O)C1Nc2ccccc2C1(C)C] | 25 |
| | N-(1-(7,8-difluoro-1-oxo-1,2-dihydroisoquinolin-4-yl)ethyl)-N,3,3- | |
| | trimethylindoline-2-carboxamide | |
| | stereoisomer IIA | |
| 106 | 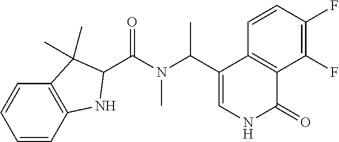 [SMILES: CC(c1c[nH]c(=O)c2c(F)c(F)ccc12)N(C)C(=O)C1Nc2ccccc2C1(C)C] | 25 |
| | N-(1-(7,8-difluoro-1-oxo-1,2-dihydroisoquinolin-4-yl)ethyl)-N,3,3- | |
| | trimethylindoline-2-carboxamide | |
| | stereoisomer IB | |
| 107 | 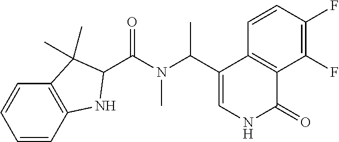 [SMILES: CC(c1c[nH]c(=O)c2c(F)c(F)ccc12)N(C)C(=O)C1Nc2ccccc2C1(C)C] | 16 |
| | N-(1-(7,8-difluoro-1-oxo-1,2-dihydroisoquinolin-4-yl)ethyl)-N,3,3- | |
| | trimethylindoline-2-carboxamide | |
| | stereoisomer IIB | |
| 108 | 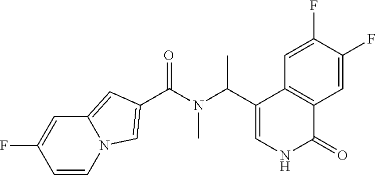 [SMILES: CC(c1c[nH]c(=O)c2cc(F)c(F)cc12)N(C)C(=O)c1cc2cc(F)ccn2c1] | 0.02 |
| | N-(1-(6,7-difluoro-1-oxo-1,2-dihydroisoquinolin-4-yl)ethyl)-7-fluoro-N- | |
| | methylindolizine-2-carboxamide | |
| | enantiomer I | |
| 109 | 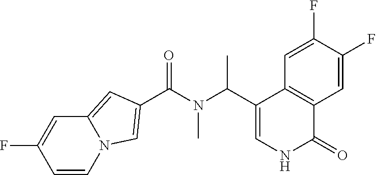 [SMILES: CC(c1c[nH]c(=O)c2cc(F)c(F)cc12)N(C)C(=O)c1cc2cc(F)ccn2c1] | 3.2 |
| | N-(1-(6,7-difluoro-1-oxo-1,2-dihydroisoquinolin-4-yl)ethyl)-7-fluoro-N- | |
| | methylindolizine-2-carboxamide | |
| | enantiomer II | |
| 110 | 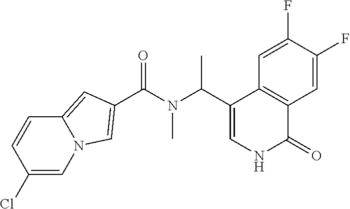 [SMILES: CC(c1c[nH]c(=O)c2cc(F)c(F)cc12)N(C)C(=O)c1cc2ccc(Cl)cn2c1] | 0.04 |
| | 6-chloro-N-(1-(6,7-difluoro-1-oxo-1,2-dihydroisoquinolin-4-yl)ethyl)-N- | |
| | methylindolizine-2-carboxamide | |
| | enantiomer I | |
| 111 | 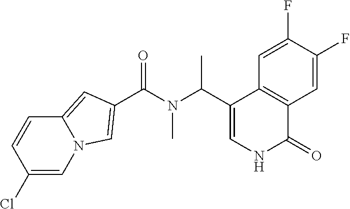 [SMILES: CC(c1c[nH]c(=O)c2cc(F)c(F)cc12)N(C)C(=O)c1cc2ccc(Cl)cn2c1] | 1.4 |
| | 6-chloro-N-(1-(6,7-difluoro-1-oxo-1,2-dihydroisoquinolin-4-yl)ethyl)-N- | |
| | methylindolizine-2-carboxamide | |
| | enantiomer II | |
| 112 | 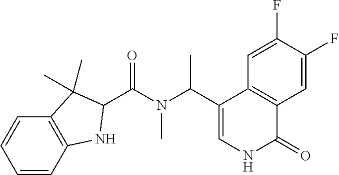 [SMILES: CC(c1c[nH]c(=O)c2cc(F)c(F)cc12)N(C)C(=O)C1Nc2ccccc2C1(C)C] | 21 |
| | N-(1-(6,7-difluoro-1-oxo-1,2-dihydroisoquinolin-4-yl)ethyl)-N,3,3- | |
| | trimethylindoline-2-carboxamide | |
| | stereoisomer IIA | |
| 113 | 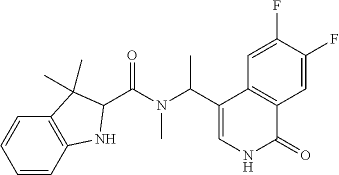 [SMILES: CC(c1c[nH]c(=O)c2cc(F)c(F)cc12)N(C)C(=O)C1Nc2ccccc2C1(C)C] | 25 |
| | N-(1-(6,7-difluoro-1-oxo-1,2-dihydroisoquinolin-4-yl)ethyl)-N,3,3- | |
| | trimethylindoline-2-carboxamide | |
| | stereoisomer IA | |
| 114 | 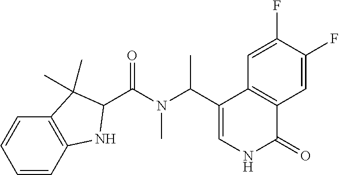 [SMILES: CC(c1c[nH]c(=O)c2cc(F)c(F)cc12)N(C)C(=O)C1Nc2ccccc2C1(C)C] | 16 |
| | N-(1-(6,7-difluoro-1-oxo-1,2-dihydroisoquinolin-4-yl)ethyl)-N,3,3- | |
| | trimethylindoline-2-carboxamide | |
| | stereoisomer IB | |
| 115 | 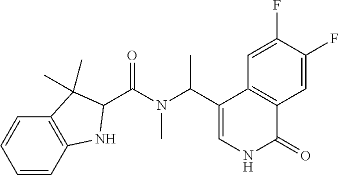 [SMILES: CC(c1c[nH]c(=O)c2cc(F)c(F)cc12)N(C)C(=O)C1Nc2ccccc2C1(C)C] | 19 |
| | N-(1-(6,7-difluoro-1-oxo-1,2-dihydroisoquinolin-4-yl)ethyl)-N,3,3- | |
| | trimethylindoline-2-carboxamide | |
| | stereoisomer IIB | |
| 116 | 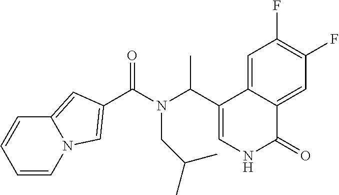 [SMILES: CC(C)CN(C(=O)c1cc2ccccn2c1)C(C)c1c[nH]c(=O)c2cc(F)c(F)cc12] | 0.08 |
| | N-(1-(6,7-Difluoro-1-oxo-1,2-dihydroisoquinolin-4-yl)ethyl)-N- | |
| | isobutylindolizine-2-carboxamide | |
| 117 | 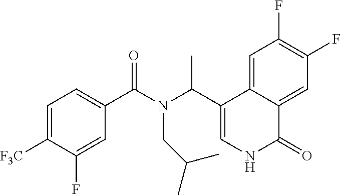 [SMILES: CC(C)CN(C(=O)c1ccc(C(F)(F)F)c(F)c1)C(C)c1c[nH]c(=O)c2cc(F)c(F)cc12] | 0.24 |
| | N-(1-(6,7-difluoro-1-oxo-1,2-dihydroisoquinolin-4-yl)ethyl)-3-fluoro-N- | |
| | isobutyl-4-(trifluoromethyl)benzamide | |
| 118 | 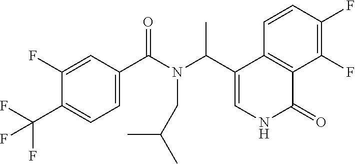 [SMILES: CC(C)CN(C(=O)c1ccc(C(F)(F)F)c(F)c1)C(C)c1c[nH]c(=O)c2c(F)c(F)ccc12] | 2.23 |
| | N-(1-(7,8-difluoro-1-oxo-1,2-dihydroisoquinolin-4-yl)ethyl)-3-fluoro-N- | |
| | isobutyl-4-(trifluoromethyl)benzamide | |
| 119 | 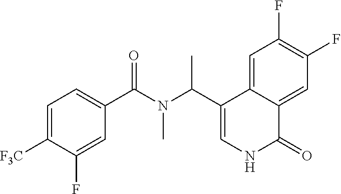 [SMILES: CC(c1c[nH]c(=O)c2cc(F)c(F)cc12)N(C)C(=O)c1ccc(C(F)(F)F)c(F)c1] | 4.02 |
| | N-(1-(6,7-Difluoro-1-oxo-1,2-dihydroisoquinolin-4-yl)ethyl)-3-fluoro-N- | |
| | methyl-4-(trifluoromethyl)benzamide | |
| | enantiomer I | |
| 120 | 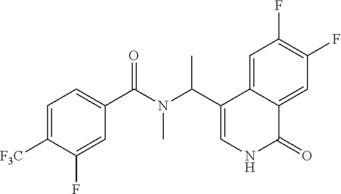 [SMILES: CC(c1c[nH]c(=O)c2cc(F)c(F)cc12)N(C)C(=O)c1ccc(C(F)(F)F)c(F)c1] | 0.02 |
| | N-(1-(6,7-Difluoro-1-oxo-1,2-dihydroisoquinolin-4-yl)ethyl)-3-fluoro-N- | |
| | methyl-4-(trifluoromethyl)benzamide | |
| | enantiomer II | |
| 121 | 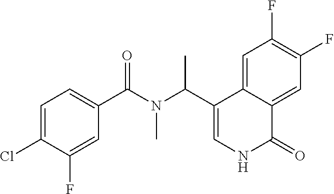 [SMILES: CC(c1c[nH]c(=O)c2cc(F)c(F)cc12)N(C)C(=O)c1ccc(Cl)c(F)c1] | 5.68 |
| | 4-Chloro-N-(1-(6,7-difluoro-1-oxo-1,2-dihydroisoquinolin-4-yl)ethyl)-3- | |
| | fluoro-N-methylbenzamide | |
| | enantiomer I | |
| 122 | 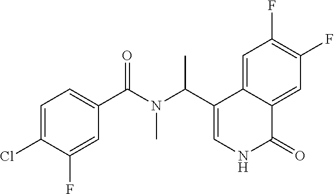 [SMILES: CC(c1c[nH]c(=O)c2cc(F)c(F)cc12)N(C)C(=O)c1ccc(Cl)c(F)c1] | 0.04 |
| | 4-Chloro-N-(1-(6,7-difluoro-1-oxo-1,2-dihydroisoquinolin-4-yl)ethyl)-3- | |
| | fluoro-N-methylbenzamide | |
| | enantiomer II | |
| 123 | 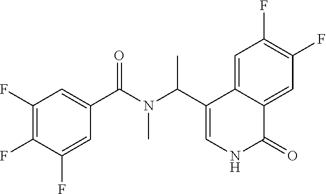 [SMILES: CC(c1c[nH]c(=O)c2cc(F)c(F)cc12)N(C)C(=O)c1cc(F)c(F)c(F)c1] | 22.41 |
| | N-(1-(6,7-Difluoro-1-oxo-1,2-dihydroisoquinolin-4-yl)ethyl)-3,4,5-trifluoro- | |
| | N-methylbenzamide | |
| | enantiomer I | |
| 124 | 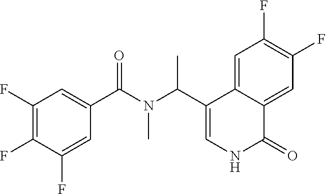 [SMILES: CC(c1c[nH]c(=O)c2cc(F)c(F)cc12)N(C)C(=O)c1cc(F)c(F)c(F)c1] | 0.19 |
| | N-(1-(6,7-Difluoro-1-oxo-1,2-dihydroisoquinolin-4-yl)ethyl)-3,4,5-trifluoro- | |
| | N-methylbenzamide | |
| | enantiomer II | |
| 125 | 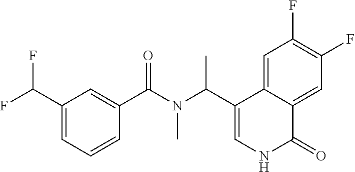 [SMILES: CC(c1c[nH]c(=O)c2cc(F)c(F)cc12)N(C)C(=O)c1cccc(C(F)F)c1] | 24.80 |
| | N-(1-(6,7-Difluoro-1-oxo-1,2-dihydroisoquinolin-4-yl)ethyl)-3- | |
| | (difluoromethyl)-N-methylbenzamide | |
| | enantiomer I | |
| 126 | 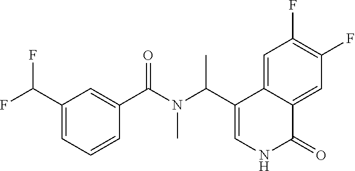 [SMILES: CC(c1c[nH]c(=O)c2cc(F)c(F)cc12)N(C)C(=O)c1cccc(C(F)F)c1] | 0.10 |
| | N-(1-(6,7-Difluoro-1-oxo-1,2-dihydroisoquinolin-4-yl)ethyl)-3- | |
| | (difluoromethyl)-N-methylbenzamide | |
| | enantiomer II | |
| 127 | 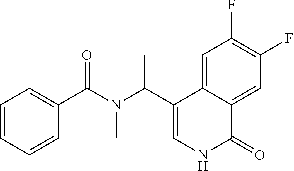 [SMILES: CC(c1c[nH]c(=O)c2cc(F)c(F)cc12)N(C)C(=O)c1ccccc1] | 3.53 |
| | N-(1-(6,7-Difluoro-1-oxo-1,2-dihydroisoquinolin-4-yl)ethyl)-N- | |
| | methylbenzamide | |
| | enantiomer I | |
| 128 | 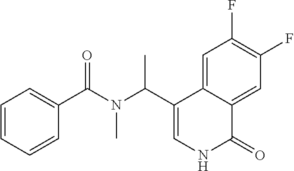 [SMILES: CC(c1c[nH]c(=O)c2cc(F)c(F)cc12)N(C)C(=O)c1ccccc1] | >25 |
| | N-(1-(6,7-Difluoro-1-oxo-1,2-dihydroisoquinolin-4-yl)ethyl)-N- | |
| | methylbenzamide | |
| | enantiomer II | |
| 129 | 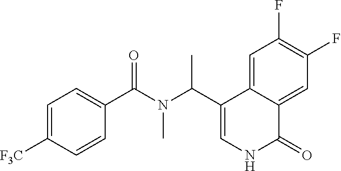 [SMILES: CC(c1c[nH]c(=O)c2cc(F)c(F)cc12)N(C)C(=O)c1ccc(C(F)(F)F)cc1] | 6.46 |
| | N-(1-(6,7-Difluoro-1-oxo-1,2-dihydroisoquinolin-4-yl)ethyl)-N-methyl-4- | |
| | (trifluoromethyl)benzamide | |
| | enantiomer I | |
| 130 | 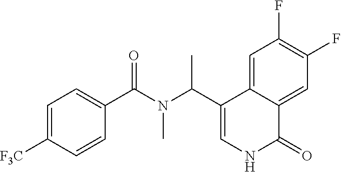 [SMILES: CC(c1c[nH]c(=O)c2cc(F)c(F)cc12)N(C)C(=O)c1ccc(C(F)(F)F)cc1] | 0.02 |
| | N-(1-(6,7-Difluoro-1-oxo-1,2-dihydroisoquinolin-4-yl)ethyl)-N-methyl-4- | |
| | (trifluoromethyl)benzamide | |
| | enantiomer II | |
| 131 | 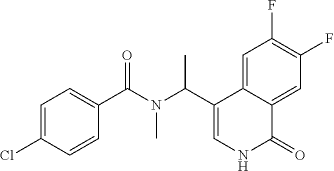 [SMILES: CC(c1c[nH]c(=O)c2cc(F)c(F)cc12)N(C)C(=O)c1ccc(Cl)cc1] | 14.68 |
| | 4-Chloro-N-(1-(6,7-difluoro-1-oxo-1,2-dihydroisoquinolin-4-yl)ethyl)-N- | |
| | methylbenzamide | |
| | enantiomer I | |
| 132 | 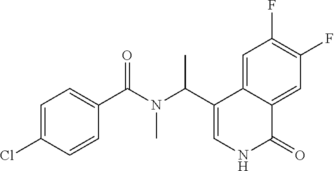 [SMILES: CC(c1c[nH]c(=O)c2cc(F)c(F)cc12)N(C)C(=O)c1ccc(Cl)cc1] | 0.06 |
| | 4-Chloro-N-(1-(6,7-difluoro-1-oxo-1,2-dihydroisoquinolin-4-yl)ethyl)-N- | |
| | methylbenzamide | |
| | enantiomer II | |
| 133 | 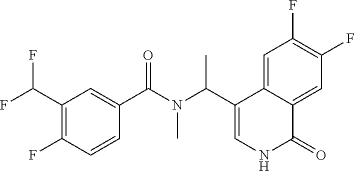 [SMILES: CC(c1c[nH]c(=O)c2cc(F)c(F)cc12)N(C)C(=O)c1ccc(F)c(C(F)F)c1] | 14.65 |
| | N-(1-(6,7-Difluoro-1-oxo-1,2-dihydroisoquinolin-4-yl)ethyl)-3- | |
| | (difluoromethyl)-4-fluoro-N-methylbenzamide | |
| | enantiomer I | |
| 134 | 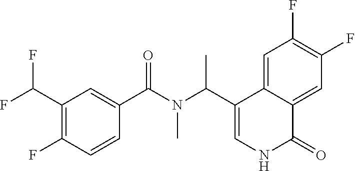 [SMILES: CC(c1c[nH]c(=O)c2cc(F)c(F)cc12)N(C)C(=O)c1ccc(F)c(C(F)F)c1] | 0.06 |
| | N-(1-(6,7-Difluoro-1-oxo-1,2-dihydroisoquinolin-4-yl)ethyl)-3- | |
| | (difluoromethyl)-4-fluoro-N-methylbenzamide | |
| | enantiomer II | |
| 135 | 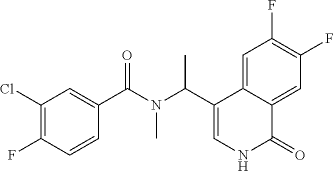 [SMILES: CC(c1c[nH]c(=O)c2cc(F)c(F)cc12)N(C)C(=O)c1ccc(F)c(Cl)c1] | 9.61 |
| | 3-Chloro-N-(1-(6,7-difluoro-1-oxo-1,2-dihydroisoquinolin-4-yl)ethyl)-4- | |
| | fluoro-N-methylbenzamide | |
| | enantiomer I | |
| 136 | 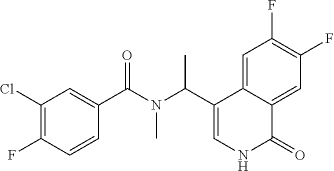 [SMILES: CC(c1c[nH]c(=O)c2cc(F)c(F)cc12)N(C)C(=O)c1ccc(F)c(Cl)c1] | 0.17 |
| | 3-Chloro-N-(1-(6,7-difluoro-1-oxo-1,2-dihydroisoquinolin-4-yl)ethyl)-4- | |
| | fluoro-N-methylbenzamide | |
| | enantiomer II | |
| 137 | 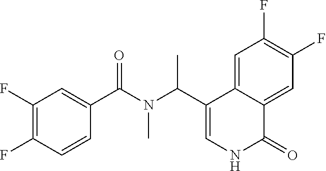 [SMILES: CC(c1c[nH]c(=O)c2cc(F)c(F)cc12)N(C)C(=O)c1ccc(F)c(F)c1] | 22.36 |
| | N-(1-(6,7-Difluoro-1-oxo-1,2-dihydroisoquinolin-4-yl)ethyl)-3,4-difluoro-N- | |
| | methylbenzamide | |
| | enantiomer I | |
| 138 | 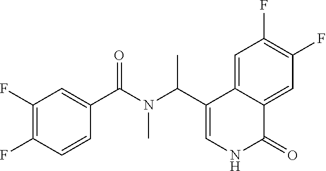 [SMILES: CC(c1c[nH]c(=O)c2cc(F)c(F)cc12)N(C)C(=O)c1ccc(F)c(F)c1] | 0.45 |
| | N-(1-(6,7-Difluoro-1-oxo-1,2-dihydroisoquinolin-4-yl)ethyl)-3,4-difluoro-N- | |
| | methylbenzamide | |
| | enantiomer II | |
| 139 | 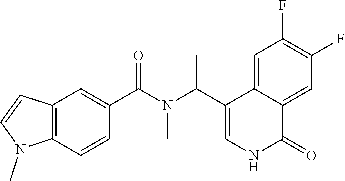 [SMILES: CC(c1c[nH]c(=O)c2cc(F)c(F)cc12)N(C)C(=O)c1ccc2c(ccn2C)c1] | >25 |
| | N-(1-(6,7-Difluoro-1-oxo-1,2-dihydroisoquinolin-4-yl)ethyl)-N,1-dimethyl- | |
| | 1H-indole-5-carboxamide | |
| | enantiomer I | |
| 140 | 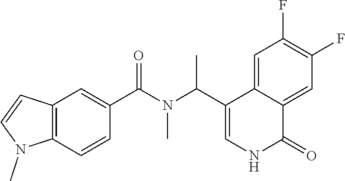 [SMILES: CC(c1c[nH]c(=O)c2cc(F)c(F)cc12)N(C)C(=O)c1ccc2c(ccn2C)c1] | 0.12 |
| | N-(1-(6,7-Difluoro-1-oxo-1,2-dihydroisoquinolin-4-yl)ethyl)-N,1-dimethyl- | |
| | 1H-indole-5-carboxamide | |
| | enantiomer II | |
| 141 | 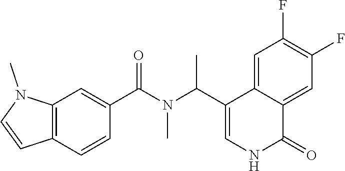 [SMILES: CC(c1c[nH]c(=O)c2cc(F)c(F)cc12)N(C)C(=O)c1ccc2ccn(C)c2c1] | >25 |
| | N-(1-(6,7-Difluoro-1-oxo-1,2-dihydroisoquinolin-4-yl)ethyl)-N,1-dimethyl- | |
| | 1H-indole-6-carboxamide | |
| | enantiomer I | |
| 142 | 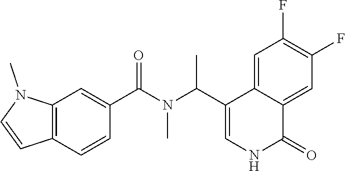 [SMILES: CC(c1c[nH]c(=O)c2cc(F)c(F)cc12)N(C)C(=O)c1ccc2ccn(C)c2c1] | 0.39 |
| | N-(1-(6,7-Difluoro-1-oxo-1,2-dihydroisoquinolin-4-yl)ethyl)-N,1-dimethyl- | |
| | 1H-indole-6-carboxamide | |
| | enantiomer II | |
| 143 | 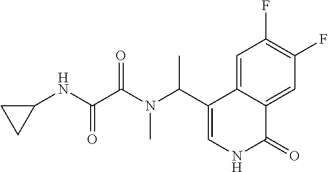 [SMILES: CC(c1c[nH]c(=O)c2cc(F)c(F)cc12)N(C)C(=O)C(=O)NC1CC1] | 25.00 |
| | N1-Cyclopropyl-N2-(1-(6,7-difluoro-1-oxo-1,2-dihydroisoquinolin-4- | |
| | yl)ethyl)-N2-methyloxalamide | |
| 144 | 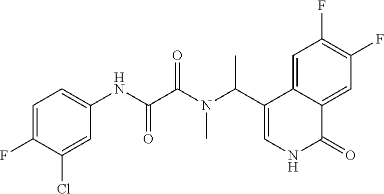 [SMILES: CC(c1c[nH]c(=O)c2cc(F)c(F)cc12)N(C)C(=O)C(=O)Nc1ccc(F)c(Cl)c1] | 1.44 |
| | N1-(3-Chloro-4-fluorophenyl)-N2-(1-(6,7-difluoro-1-oxo-1,2- | |
| | dihydroisoquinolin-4-yl)ethyl)-N2-methyloxalamide | |
| 145 | 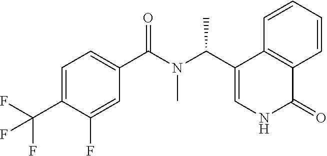 [SMILES: C[C@H](c1c[nH]c(=O)c2ccccc12)N(C)C(=O)c1ccc(C(F)(F)F)c(F)c1] | 0.11 |
| | (R)-3-fluoro-N-methyl-N-(1-(1-oxo-1,2-dihydroisoquinolin-4-yl)ethyl)-4- | |
| | (trifluoromethyl)benzamide | |
| 146 | 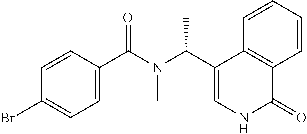 [SMILES: C[C@H](c1c[nH]c(=O)c2ccccc12)N(C)C(=O)c1ccc(Br)cc1] | 0.15 |
| | (R)-4-bromo-N-methyl-N-(1-(1-oxo-1,2-dihydroisoquinolin-4- | |
| | yl)ethyl)benzamide | |
| 147 | 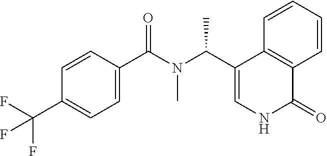 [SMILES: C[C@H](c1c[nH]c(=O)c2ccccc12)N(C)C(=O)c1ccc(C(F)(F)F)cc1] | 0.16 |
| | (R)-N-methyl-N-(1-(1-oxo-1,2-dihydroisoquinolin-4-yl)ethyl)-4- | |
| | (trifluoromethyl)benzamide | |
| 148 | 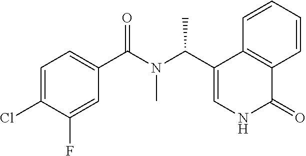 [SMILES: C[C@H](c1c[nH]c(=O)c2ccccc12)N(C)C(=O)c1ccc(Cl)c(F)c1] | 0.27 |
| | (R)-4-chloro-3-fluoro-N-methyl-N-(1-(1-oxo-1,2-dihydroisoquinolin-4- | |
| | yl)ethyl)benzamide | |
| 149 | 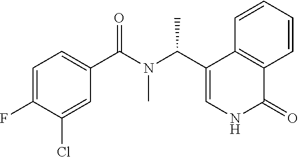 [SMILES: C[C@H](c1c[nH]c(=O)c2ccccc12)N(C)C(=O)c1ccc(F)c(Cl)c1] | 0.68 |
| | (R)-3-chloro-4-fluoro-N-methyl-N-(1-(1-oxo-1,2-dihydroisoquinolin-4- | |
| | yl)ethyl)benzamide | |
| 150 | 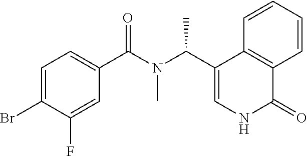 [SMILES: C[C@H](c1c[nH]c(=O)c2ccccc12)N(C)C(=O)c1ccc(Br)c(F)c1] | 0.51 |
| | (R)-4-bromo-3-fluoro-N-methyl-N-(1-(1-oxo-1,2-dihydroisoquinolin-4- | |
| | yl)ethyl)benzamide | |
| 151 | 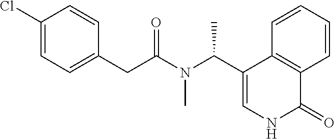 [SMILES: C[C@H](c1c[nH]c(=O)c2ccccc12)N(C)C(=O)Cc1ccc(Cl)cc1] | 3.54 |
| | (R)-2-(4-chlorophenyl)-N-methyl-N-(1-(1-oxo-1,2-dihydroisoquinolin-4- | |
| | yl)ethyl)acetamide | |
| 152 | 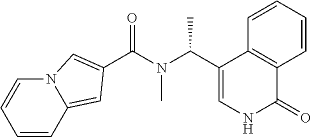 [SMILES: C[C@H](c1c[nH]c(=O)c2ccccc12)N(C)C(=O)c1cc2ccccn2c1] | 0.47 |
| | (R)-N-methyl-N-(1-(1-oxo-1,2-dihydroisoquinolin-4-yl)ethyl)indolizine-2- | |
| | carboxamide | |
| 153 | 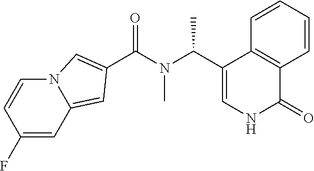 [SMILES: C[C@H](c1c[nH]c(=O)c2ccccc12)N(C)C(=O)c1cc2cc(F)ccn2c1] | 0.35 |
| | (R)-7-fluoro-N-methyl-N-(1-(1-oxo-1,2-dihydroisoquinolin-4- | |
| | yl)ethyl)indolizine-2-carboxamide | |
| 154 | 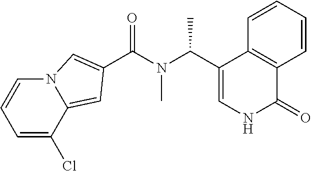 [SMILES: C[C@H](c1c[nH]c(=O)c2ccccc12)N(C)C(=O)c1cc2c(Cl)cccn2c1] | 0.14 |
| | (R)-8-chloro-N-methyl-N-(1-(1-oxo-1,2-dihydroisoquinolin-4- | |
| | yl)ethyl)indolizine-2-carboxamide | |
| 155 | 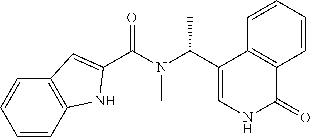 [SMILES: C[C@H](c1c[nH]c(=O)c2ccccc12)N(C)C(=O)c1cc2ccccc2[nH]1] | 0.17 |
| | (R)-N-methyl-N-(1-(1-oxo-1,2-dihydroisoquinolin-4-yl)ethyl)-1H-indole-2- | |
| | carboxamide | |
| 156 | 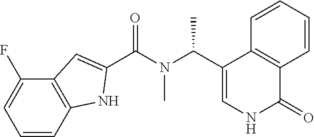 [SMILES: C[C@H](c1c[nH]c(=O)c2ccccc12)N(C)C(=O)c1cc2c(F)cccc2[nH]1] | 0.12 |
| | (R)-4-fluoro-N-methyl-N-(1-(1-oxo-1,2-dihydroisoquinolin-4-yl)ethyl)-1H- | |
| | indole-2-carboxamide | |
| 157 | 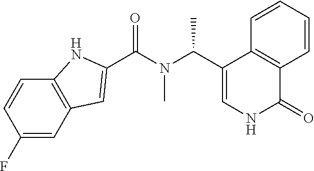 [SMILES: C[C@H](c1c[nH]c(=O)c2ccccc12)N(C)C(=O)c1cc2cc(F)ccc2[nH]1] | 0.15 |
| | (R)-5-fluoro-N-methyl-N-(1-(1-oxo-1,2-dihydroisoquinolin-4-yl)ethyl)-1H- | |
| | indole-2-carboxamide | |
| 158 | 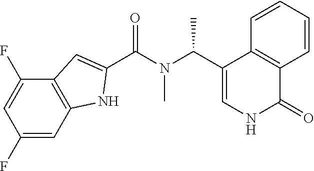 [SMILES: C[C@H](c1c[nH]c(=O)c2ccccc12)N(C)C(=O)c1cc2c(F)cc(F)cc2[nH]1] | 0.13 |
| | (R)-4,6-difluoro-N-methyl-N-(1-(1-oxo-1,2-dihydroisoquinolin-4-yl)ethyl)- | |
| | 1H-indole-2-carboxamide | |
| 159 | 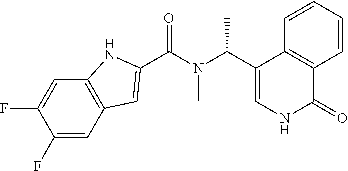 [SMILES: C[C@H](c1c[nH]c(=O)c2ccccc12)N(C)C(=O)c1cc2cc(F)c(F)cc2[nH]1] | 0.10 |
| | (R)-5,6-difluoro-N-methyl-N-(1-(1-oxo-1,2-dihydroisoquinolin-4-yl)ethyl)- | |
| | 1H-indole-2-carboxamide | |
| 160 | 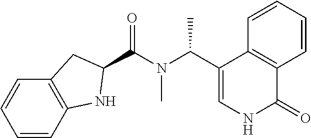 [SMILES: C[C@H](c1c[nH]c(=O)c2ccccc12)N(C)C(=O)[C@@H]1Cc2ccccc2N1] | 0.39 |
| | (S)-N-methyl-N-((R)-1-(1-oxo-1,2-dihydroisoquinolin-4-yl)ethyl)indoline-2- | |
| | carboxamide | |
| 161 | 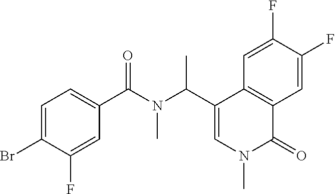 [SMILES: CC(c1cn(C)c(=O)c2cc(F)c(F)cc12)N(C)C(=O)c1ccc(Br)c(F)c1] | 3.09 |
| | 4-bromo-N-(1-(6,7-difluoro-2-methyl-1-oxo-1,2-dihydroisoquinolin-4- | |
| | yl)ethyl)-3-fluoro-N-methylbenzamide | |
| | enantiomer I | |
| 162 | 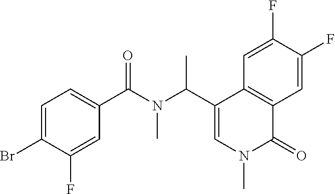 [SMILES: CC(c1cn(C)c(=O)c2cc(F)c(F)cc12)N(C)C(=O)c1ccc(Br)c(F)c1] | 0.03 |
| | 4-bromo-N-(1-(6,7-difluoro-2-methyl-1-oxo-1,2-dihydroisoquinolin-4- | |
| | yl)ethyl)-3-fluoro-N-methylbenzamide | |
| | enantiomer II | |
| 163 | 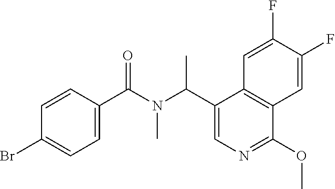 [SMILES: COc1ncc(C(C)N(C)C(=O)c2ccc(Br)cc2)c2cc(F)c(F)cc12] | 0.44 |
| | 4-bromo-N-(1-(6,7-difluoro-1-methoxyisoquinolin-4-yl)ethyl)-N- | |
| | methylbenzamide | |
| | enantiomer I | |
| 164 | 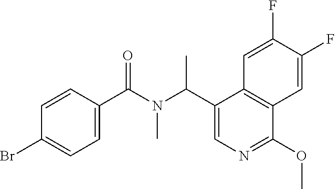 [SMILES: COc1ncc(C(C)N(C)C(=O)c2ccc(Br)cc2)c2cc(F)c(F)cc12] | 17.19 |
| | 4-bromo-N-(1-(6,7-difluoro-1-methoxyisoquinolin-4-yl)ethyl)-N- | |
| | methylbenzamide | |
| | enantiomer II | |
| 165 | 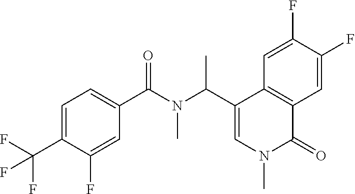 [SMILES: CC(c1cn(C)c(=O)c2cc(F)c(F)cc12)N(C)C(=O)c1ccc(C(F)(F)F)c(F)c1] | 2.34 |
| | N-(1-(6,7-difluoro-2-methyl-1-oxo-1,2-dihydroisoquinolin-4-yl)ethyl)-3- | |
| | fluoro-N-methyl-4-(trifluoromethyl)benzamide | |
| | enantiomer I | |
| 166 | 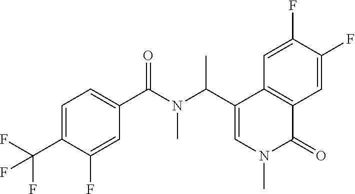 [SMILES: CC(c1cn(C)c(=O)c2cc(F)c(F)cc12)N(C)C(=O)c1ccc(C(F)(F)F)c(F)c1] | 3.32 |
| | N-(1-(6,7-difluoro-2-methyl-1-oxo-1,2-dihydroisoquinolin-4-yl)ethyl)-3- | |
| | fluoro-N-methyl-4-(trifluoromethyl)benzamide | |
| | enantiomer II | |
| 167 | 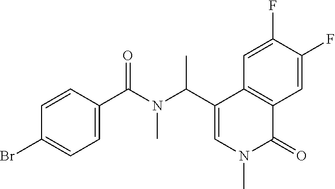 [SMILES: CC(c1cn(C)c(=O)c2cc(F)c(F)cc12)N(C)C(=O)c1ccc(Br)cc1] | 2.32 |
| | 4-bromo-N-(1-(6,7-difluoro-2-methyl-1-oxo-1,2-dihydroisoquinolin-4- | |
| | yl)ethyl)-N-methylbenzamide | |
| | enantiomer I | |
| 168 | 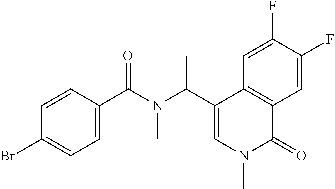 [SMILES: CC(c1cn(C)c(=O)c2cc(F)c(F)cc12)N(C)C(=O)c1ccc(Br)cc1] | 0.03 |
| | 4-bromo-N-(1-(6,7-difluoro-2-methyl-1-oxo-1,2-dihydroisoquinolin-4- | |
| | y1)ethyl)-N-methylbenzamide | |
| | enantiomer II | |
| 169 | 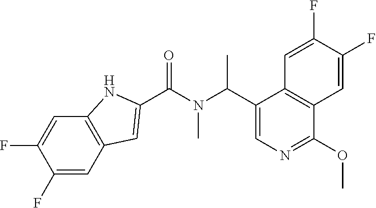 [SMILES: COc1ncc(C(C)N(C)C(=O)c2cc3cc(F)c(F)cc3[nH]2)c2cc(F)c(F)cc12] | 0.13 |
| | N-(1-(6,7-difluoro-1-methoxyisoquinolin-4-yl)ethyl)-5,6-difluoro-N-methyl- | |
| | 1H-indole-2-carboxamide | |
| | enantiomer I | |
| 170 | 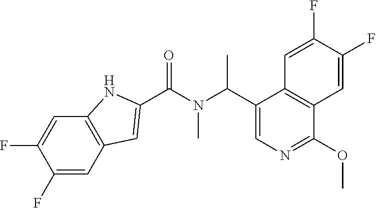 [SMILES: COc1ncc(C(C)N(C)C(=O)c2cc3cc(F)c(F)cc3[nH]2)c2cc(F)c(F)cc12] | 5.04 |
| | N-(1-(6,7-difluoro-1-methoxyisoquinolin-4-yl)ethyl)-5,6-difluoro-N-methyl- | |
| | 1H-indole-2-carboxamide | |
| | enantiomer II | |
| 171 | 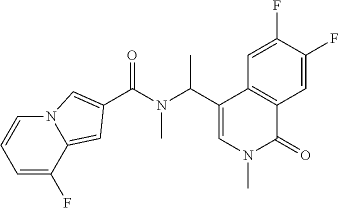 [SMILES: CC(c1cn(C)c(=O)c2cc(F)c(F)cc12)N(C)C(=O)c1cc2c(F)cccn2c1] | 16.43 |
| | N-(1-(6,7-difluoro-2-methyl-1-oxo-1,2-dihydroisoquinolin-4-yl)ethyl)-8- | |
| | fluoro-N-methylindolizine-2-carboxamide | |
| | enantiomer I | |
| 172 | 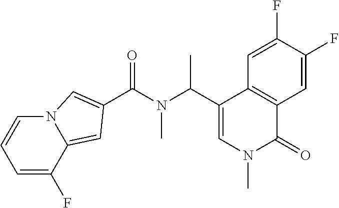 [SMILES: CC(c1cn(C)c(=O)c2cc(F)c(F)cc12)N(C)C(=O)c1cc2c(F)cccn2c1] | 0.09 |
| | N-(1-(6,7-difluoro-2-methyl-1-oxo-1,2-dihydroisoquinolin-4-yl)ethyl)-8- | |
| | fluoro-N-methylindolizine-2-carboxamide | |
| 173 | 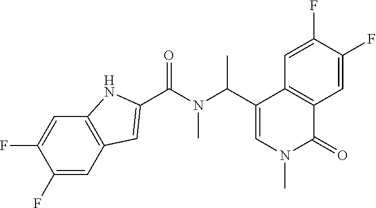 [SMILES: CC(c1cn(C)c(=O)c2cc(F)c(F)cc12)N(C)C(=O)c1cc2cc(F)c(F)cc2[nH]1] | 2.12 |
| | N-(1-(6,7-difluoro-2-methyl-1-oxo-1,2-dihydroisoquinolin-4-yl)ethyl)-5,6- | |
| | difluoro-N-methyl-1H-indole-2-carboxamide | |
| | enantiomer I | |
| 174 | 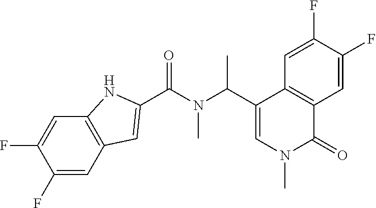 [SMILES: CC(c1cn(C)c(=O)c2cc(F)c(F)cc12)N(C)C(=O)c1cc2cc(F)c(F)cc2[nH]1] | 0.05 |
| | N-(1-(6,7-difluoro-2-methyl-1-oxo-1,2-dihydroisoquinolin-4-yl)ethyl)-5,6- | |
| | difluoro-N-methyl-1H-indole-2-carboxamide | |
| | enantiomer I | |
| 175 | 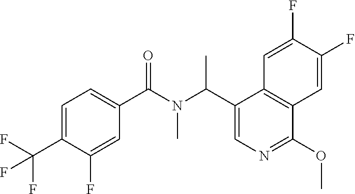 [SMILES: COc1ncc(C(C)N(C)C(=O)c2ccc(C(F)(F)F)c(F)c2)c2cc(F)c(F)cc12] | 24.28 |
| | N-(1-(6,7-difluoro-1-methoxyisoquinolin-4-yl)ethyl)-3-fluoro-N-methyl-4- | |
| | (trifluoromethyl)benzamide | |
| | enantiomer I | |
| 176 | 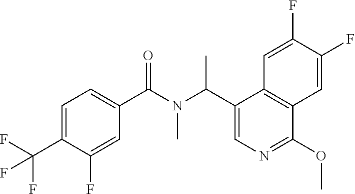 [SMILES: COc1ncc(C(C)N(C)C(=O)c2ccc(C(F)(F)F)c(F)c2)c2cc(F)c(F)cc12] | 0.56 |
| | N-(1-(6,7-difluoro-1-methoxyisoquinolin-4-yl)ethyl)-3-fluoro-N-methyl-4- | |
| | (trifluoromethyl)benzamide | |
| | enantiomer II | |
| 177 | 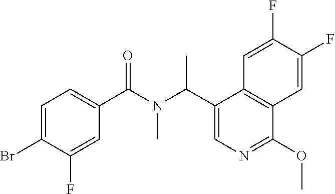 [SMILES: COc1ncc(C(C)N(C)C(=O)c2ccc(Br)c(F)c2)c2cc(F)c(F)cc12] | 20.20 |
| | 4-bromo-N-(1-(6,7-difluoro-1-methoxyisoquinolin-4-yl)ethyl)-3-fluoro-N- | |
| | methylbenzamide | |
| | enantiomer I | |
| 178 | 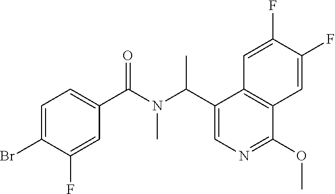 [SMILES: COc1ncc(C(C)N(C)C(=O)c2ccc(Br)c(F)c2)c2cc(F)c(F)cc12] | 0.20 |
| | 4-bromo-N-(1-(6,7-difluoro-1-methoxyisoquinolin-4-yl)ethyl)-3-fluoro-N- | |
| | methylbenzamide | |
| | enantiomer II | |
| 179 | 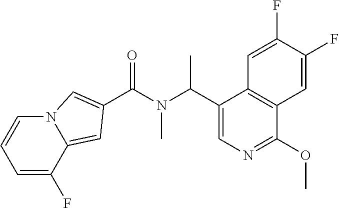 [SMILES: COc1ncc(C(C)N(C)C(=O)c2cc3c(F)cccn3c2)c2cc(F)c(F)cc12] | 9.38 |
| | N-(1-(6,7-difluoro-1-methoxyisoquinolin-4-yl)ethyl)-8-fluoro-N- | |
| | methylindolizine-2-carboxamide | |
| | enantiomer I | |
| 180 | 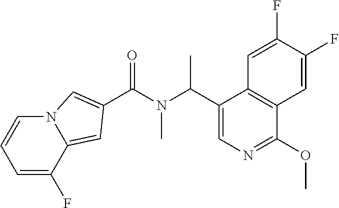 [SMILES: COc1ncc(C(C)N(C)C(=O)c2cc3c(F)cccn3c2)c2cc(F)c(F)cc12] | 0.24 |
| | N-(1-(6,7-difluoro-1-methoxyisoquinolin-4-yl)ethyl)-8-fluoro-N- | |
| | methylindolizine-2-carboxamide | |
| | enantiomer II | |
| 181 | 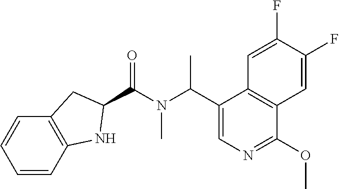 [SMILES: COc1ncc(C(C)N(C)C(=O)[C@@H]2Cc3ccccc3N2)c2cc(F)c(F)cc12] | 0.29 |
| | (2S)-N-(1-(6,7-difluoro-1-methoxyisoquinolin-4-yl)ethyl)-N-methylindoline- | |
| | 2-carboxamide | |
| | diastereomer I | |
| 182 | 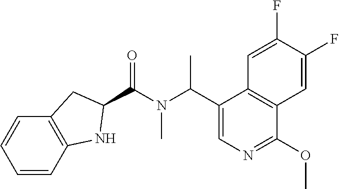 [SMILES: COc1ncc(C(C)N(C)C(=O)[C@@H]2Cc3ccccc3N2)c2cc(F)c(F)cc12] | 24.72 |
| | (2S)-N-(1-(6,7-difluoro-1-methoxyisoquinolin-4-yl)ethyl)-N-methylindoline- | |
| | 2-carboxamide | |
| | diastereomer II | |
| 183 | 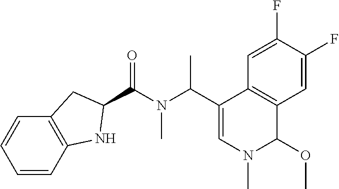 [SMILES: COC1c2cc(F)c(F)cc2C(C(C)N(C)C(=O)[C@@H]2Cc3ccccc3N2)=CN1C] | 0.07 |
| | (2S)-N-(1-(6,7-difluoro-2-methyl-1-oxo-1,2-dihydroisoquinolin-4-yl)ethyl)- | |
| | N-methylindoline-2-carboxamide | |
| | diastereomer I | |
| 184 | 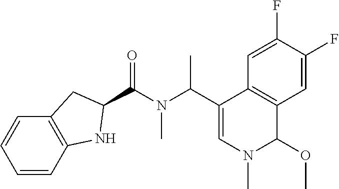 [SMILES: COC1c2cc(F)c(F)cc2C(C(C)N(C)C(=O)[C@@H]2Cc3ccccc3N2)=CN1C] | 4.20 |
| | (2S)-N-(1-(6,7-difluoro-2-methyl-1-oxo-1,2-dihydroisoquinolin-4-yl)ethyl)- | |
| | N-methylindoline-2-carboxamide | |
| | diastereomer II | |
| 185 | 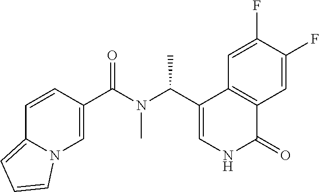 [SMILES: C[C@H](c1c[nH]c(=O)c2cc(F)c(F)cc12)N(C)C(=O)c1ccc2cccn2c1] | 0.04 |
| | (R)-N-(1-(6,7-difluoro-1-oxo-1,2-dihydroisoquinolin-4-yl)ethyl)-N- | |
| | methylindolizine-6-carboxamide | |
| 186 | 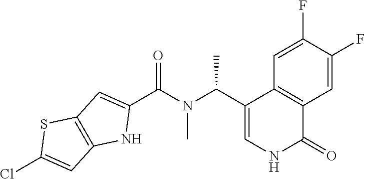 [SMILES: C[C@H](c1c[nH]c(=O)c2cc(F)c(F)cc12)N(C)C(=O)c1cc2sc(Cl)cc2[nH]1] | 0.03 |
| | (R)-2-chloro-N-(1-(6,7-difluoro-1-oxo-1,2-dihydroisoquinolin-4-yl)ethyl)-N- | |
| | methyl-4H-thieno[3,2-b]pyrrole-5-carboxamide | |
| 187 | 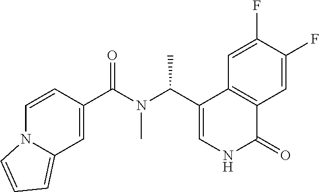 [SMILES: C[C@H](c1c[nH]c(=O)c2cc(F)c(F)cc12)N(C)C(=O)c1ccn2cccc2c1] | 0.18 |
| | (R)-N-(1-(6,7-difluoro-1-oxo-1,2-dihydroisoquinolin-4-yl)ethyl)-N- | |
| | methylindolizine-7-carboxamide | |

### Enumerated Embodiments

The following exemplary embodiments are provided, the numbering of which is not to be construed as designating levels of importance:

Embodiment 1 provides a compound of formula (I), or a salt, solvate, prodrug, stereoisomer, tautomer, or isotopically labeled derivative thereof, or any mixtures thereof:

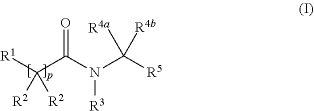

wherein:
R¹ is selected from the group consisting of

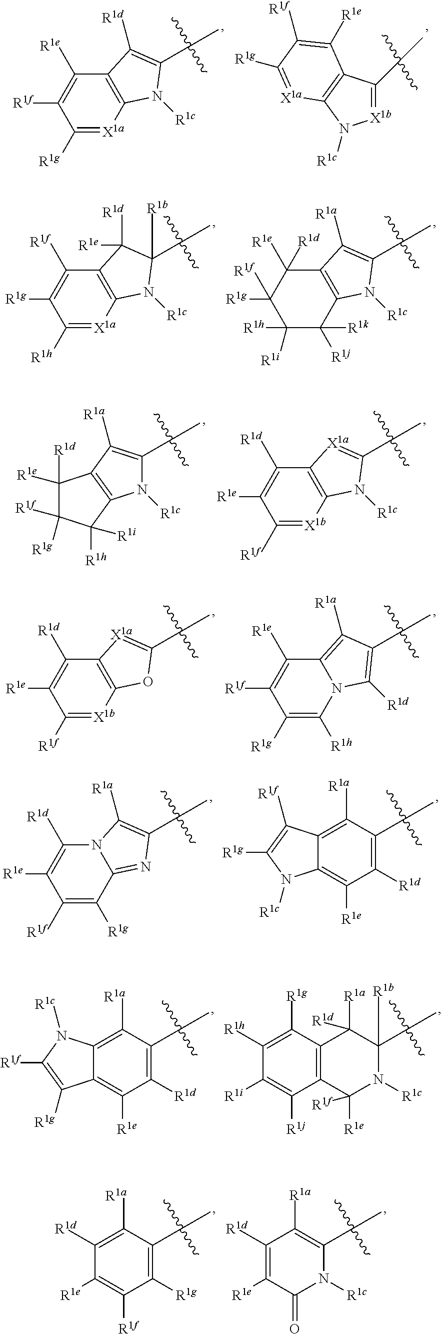

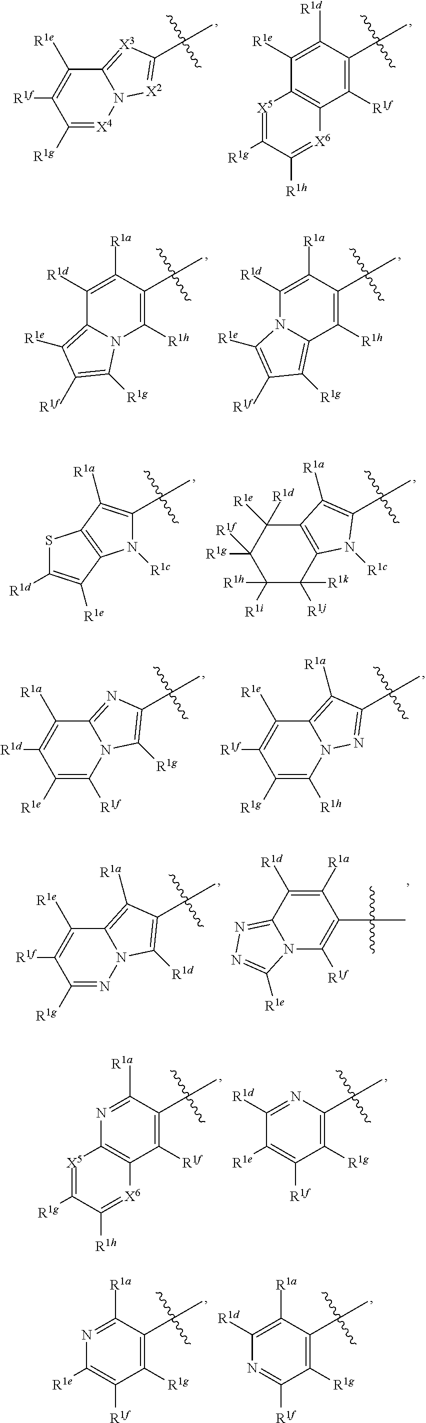

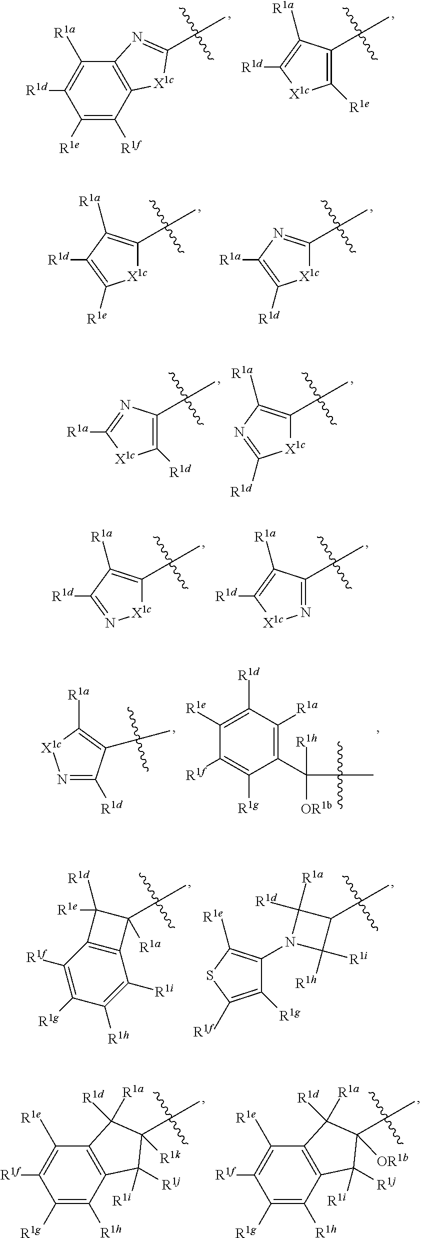

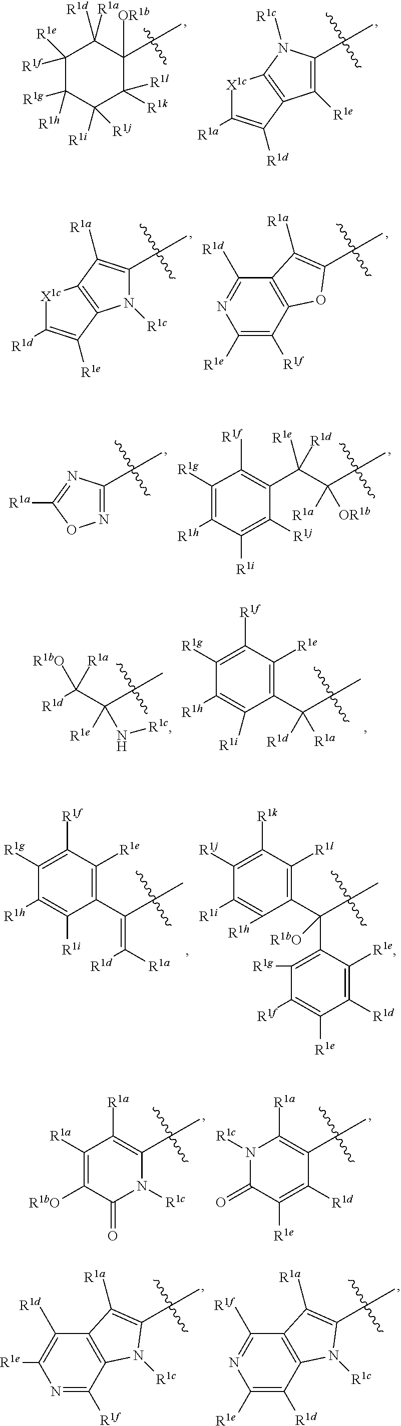

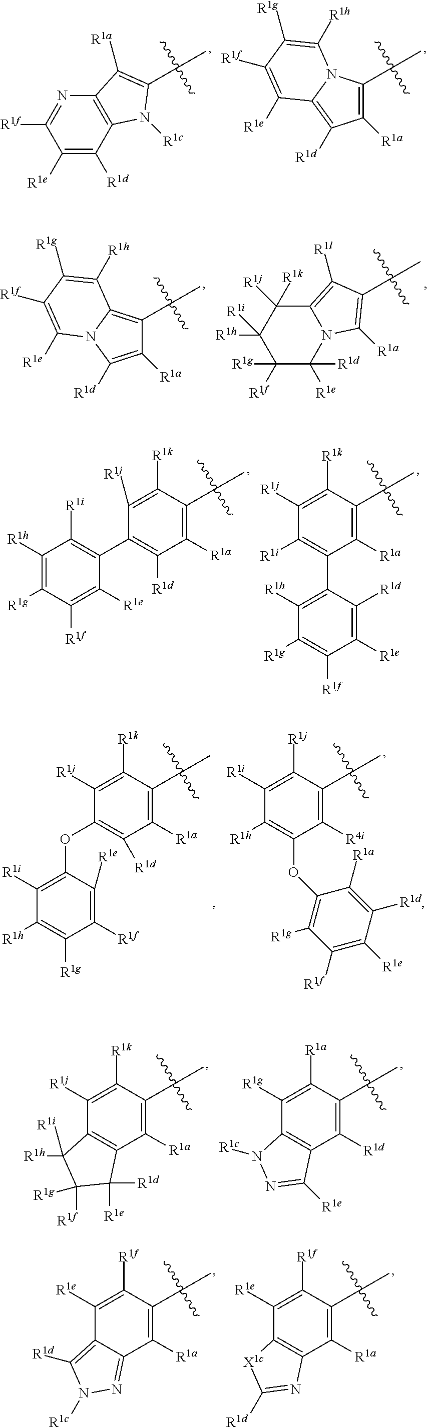

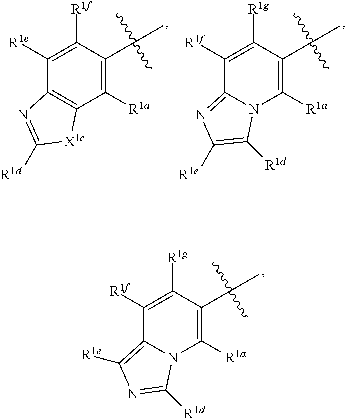

optionally substituted C₃-C₈ cycloalkyl, —NH(optionally substituted C₃-C₈ cycloalkyl), and —NH(optionally substituted phenyl);
each occurrence of R^{1a}, R^{1d}, R^{1e}, R^{1f}, R^{1g}, R^{1h}, R¹ⁱ, R^{1j}, R^{1k} and R^{1l} is independently selected from the group consisting of H, halogen, —CN, optionally substituted C₁-C₆ alkyl, optionally substituted C₃-C₈ cycloalkyl, optionally substituted C₁-C₆ alkoxy, optionally substituted C₃-C₈ cycloalkoxy, heterocyclyl, heteroaryl, —S(optionally substituted C₁-C₆ alkyl), —SO(optionally substituted C₁-C₆ alkyl), —SO₂(optionally substituted C₁-C₆ alkoxy), —C(═O)OH, —C(═O)O(optionally substituted C₁-C₆ alkyl), —C(═O)O(optionally substituted C₃-C₈ cycloalkyl), —O(optionally substituted C₁-C₆ alkyl), —O(optionally substituted C₃-C₈ cycloalkyl), —NH₂, —NH(optionally substituted C₁-C₆ alkyl), —NH(optionally substituted C₃-C₈ cycloalkyl), —N(optionally substituted C₁-C₆ alkyl)(optionally substituted C₁-C₆ alkyl), —N(optionally substituted C₃-C₈ cycloalkyl)(optionally substituted C₃-C₈ cycloalkyl), —N(optionally substituted C₁-C₆ alkyl)(optionally substituted C₃-C₈ cycloalkyl), —C(═O)NH₂, —C(═O)NH(optionally substituted C₁-C₆ alkyl), —C(═O)NH(optionally substituted C₃-C₈ cycloalkyl), —C(═O)N(optionally substituted C₁-C₆ alkyl)(optionally substituted C₁-C₆ alkyl), —C(═O)N(optionally substituted C₃-C₈ cycloalkyl)(optionally substituted C₃-C₈ cycloalkyl), and —C(═O)N(optionally substituted C₁-C₆ alkyl)(optionally substituted C₃-C₈ cycloalkyl;
each occurrence of R^{1b} is independently H, optionally substituted C₁-C₆ alkyl, or optionally substituted C₃-C₈ cycloalkyl;
each occurrence of R^{1c} is independently H, optionally substituted C₁-C₆ alkyl, or optionally substituted C₃-C₈ cycloalkyl;
each occurrence of X^{1a} is independently N or C(R^{1a});
each occurrence of X^{1b} is independently N or C(R^{1d});
each occurrence of X^{1c} is independently N(R^{1b}), 0, or S;
one of the following applies:
   (i) X² is N, X³ is C(R^{1a}), and X⁴ is C(R^{1d});
   (ii) X³ is N, X² is C(R^{1a}), and X⁴ is C(R^{1d}); or
   (iii) X⁴ is N, X² is C(R^{1a}), and X³ is C(R^{1d});
one of the following applies:
   (i) X⁵ is N and X⁶ is C(R^{1a}); or
   (ii) X⁶ is N and X⁵ is C(R^{1a});
each occurrence of R² is independently H, optionally substituted C₁-C₆ alkyl, or optionally substituted C₃-C₈ cycloalkyl, or the two R² groups combine to form ═O;
R³ is selected from the group consisting of H, C₁-C₆ alkyl, and C₃-C₈ cycloalkyl, wherein the alkyl or cycloalkyl is optionally substituted with at least one substituent selected from the group consisting of C₁-C₆ alkyl, C₃-C₈ cycloalkyl, halogen, cyano, —OH, C₁-C₆ alkoxy, C₃-C₈ cycloalkoxy, C₁-C₆ haloalkoxy, C₃-C₈ halocycloalkoxy, optionally substituted phenyl, optionally substituted heteroaryl, optionally substituted heterocyclyl, —C(═O)OR⁶, —OC(═O)R⁶, —SR⁶, —S(═O)R⁶, —S(═O)₂R⁶, —S(═O)₂NR⁶R⁶, —N(R⁶)S(═O)₂R⁶, —N(R⁶)C(═O)R⁶, —C(═O)NR⁶R⁶, and —NR⁶R⁶.
R^{4a} is selected from the group consisting of H, C₁-C₆ alkyl, C₃-C₈ cycloalkyl, and phenyl, wherein the alkyl, cycloalkyl, or phenyl is optionally substituted with at least one substituent selected from the group consisting of C₁-C₆ alkyl, C₃-C₈ cycloalkyl, halogen, cyano, —OH, C₁-C₆ alkoxy, C₃-C₅ cycloalkoxy, C₁-C₆ haloalkoxy, C₃-C₅ halocycloalkoxy, —NR⁶R⁶, and optionally substituted phenyl;
R^{4b} selected from the group consisting of H and optionally substituted C₁-C₆ alkyl;
R⁵ is selected from the group consisting of

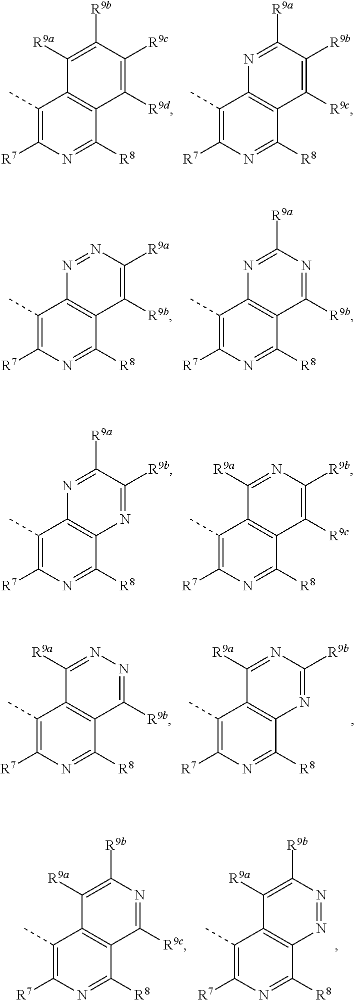

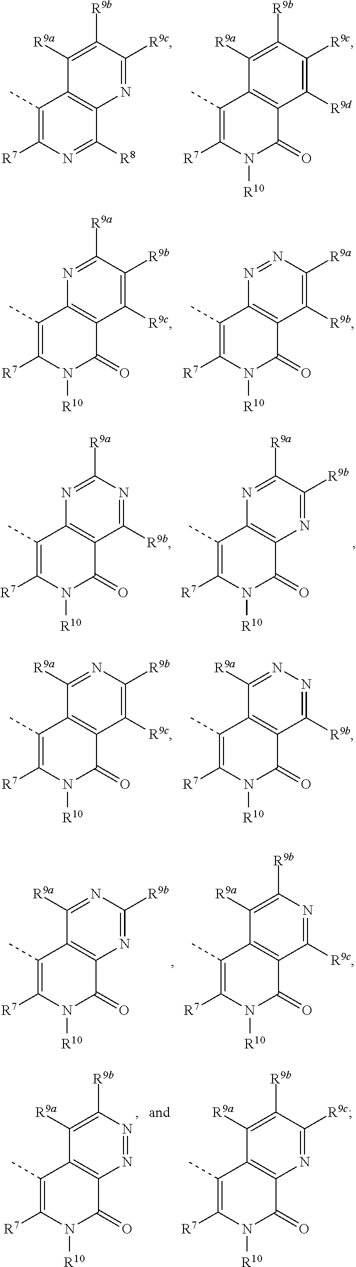

each occurrence of R⁶ is independently selected from the group consisting of H, optionally substituted C₁-C₆ alkyl, optionally substituted C₃-C₈ cycloalkyl, optionally substituted phenyl, and optionally substituted hetereoaryl;
each occurrence of R⁷ is independently selected from the group consisting of H, halogen, optionally substituted C₁-C₆ alkyl, optionally substituted C₃-C₈ cycloalkyl, optionally substituted C₁-C₆ alkoxy, and optionally substituted C₃-C₈ cycloalkoxy;
each occurrence of R⁷ is independently selected from the group consisting of H, halogen, —CN, optionally substituted C₁-C₆ alkyl, optionally substituted C₃-C₈ cycloalkyl, optionally substituted C₁-C₆ alkoxy, optionally substituted C₃-C₈ cycloalkoxy, heterocyclyl, heteroaryl, —S(optionally substituted C₁-C₆ alkyl), —SO(optionally substituted C₁-C₆ alkyl), —SO₂(optionally substituted C₁-C₆ alkyl), —C(═O)OH, —C(═O)O(optionally substituted C₁-C₆ alkyl), —C(═O)O(optionally substituted C₃-C₈ cycloalkyl), —O(optionally substituted C₁-C₆ alkyl), —O(optionally substituted C₃-C₈ cycloalkyl), —NH₂, —NH(optionally substituted C₁-C₆ alkyl), —NH(optionally substituted C₃-C₈ cycloalkyl), —N(optionally substituted C₁-C₆ alkyl)(optionally substituted C₁-C₆ alkyl), —N(optionally substituted C₃-C₈ cycloalkyl)(optionally substituted C₃-C₈ cycloalkyl), —N(optionally substituted C₁-C₆ alkyl)(optionally substituted C₃-C₈ cycloalkyl), —C(═O)NH₂, —C(═O)NH(optionally substituted C₁-C₆ alkyl), —C(═O)NH(optionally substituted C₃-C₈ cycloalkyl), —C(═O)N(optionally substituted C₁-C₆ alkyl)(optionally substituted C₁-C₆ alkyl), —C(═O)N(optionally substituted C₃-C₈ cycloalkyl)(optionally substituted C₃-C₈ cycloalkyl), and —C(═O)N(optionally substituted C₁-C₆ alkyl)(optionally substituted C₃-C₈ cycloalkyl;
each occurrence of R^{9a}, R^{9b}, R^{9c}, and R^{9d} is independently selected from the group consisting of H, halogen, optionally substituted C₁-C₆ alkyl, optionally substituted C₃-C₈ cycloalkyl, optionally substituted C₁-C₆ alkoxy, and optionally substituted C₃-C₈ cycloalkoxy;
each occurrence of R¹⁰ is independently selected from the group consisting of H, optionally substituted C₁-C₆ alkyl, and optionally substituted C₃-C₈ cycloalkyl; and
each occurrence of p is 0 or 1,
   wherein if p is 0, then R¹ is not —NH(optionally substituted C₃-C₈ cycloalkyl) or —NH(optionally substituted phenyl).

Embodiment 2 provides the compound of Embodiment 1, wherein each occurrence of aryl or heteroaryl is independently optionally substituted with at least one substituent selected from the group consisting of C₁-C₆ alkyl, C₃-C₈ cycloalkyl, phenyl, C₁-C₆ hydroxyalkyl, (C₁-C₆ alkoxy)-C₁-C₆ alkyl, C₁-C₆ haloalkyl, C₁-C₆ haloalkoxy, halogen, —CN, —OR^{b}, —N(R^{b})(R^{b}), —NO₂, —C(═O)N(R^{b})(R^{b}), —C(═O)OR^{b}, —OC(═O)R^{b}, —SR^{b}, —S(═O)R^{b}, —S(═O)₂R^{b}, N(R^{b})S(═O)₂R^{b}, —S(═O)₂N(R^{b})(R^{b}), acyl, and C₁-C₆ alkoxycarbonyl, wherein each occurrence of R^{b} is independently H, C₁-C₆ alkyl, or C₃-C₈ cycloalkyl, wherein in R^{b} the alkyl or cycloalkyl is optionally substituted with at least one selected from the group consisting of halogen, —OH, C₁-C₆ alkoxy, and heteroaryl; or substituents on two adjacent carbon atoms combine to form —O(CH₂)₁₋₃O—.

Embodiment 3 provides the compound of any of Embodiments 1-2, wherein each occurrence of alkyl, alkenyl, alkynyl, or cycloalkyl is independently optionally substituted with at least one substituent selected from the group consisting of C₁-C₆ alkyl, C₃-C₈ cycloalkyl, halo, cyano (—CN), —OR^{a}, optionally substituted phenyl, optionally substituted heteroaryl, optionally substituted heterocyclyl, —C(═O)OR^{a}, —OC(═O)R^{a}, —SR^{a}, —S(═O)R^{a}, —S(═O)₂R^{a}, —S(═O)₂NR^{a}R^{a}, —N(R^{a})S(═O)₂R^{a}, —N(R^{a})C(═O)R^{a}, —C(═O)NR^{a}R^{a}, and —N(R^{a})(R^{a}), wherein each occurrence of R^{a} is independently H, optionally substituted C₁-C₆ alkyl, optionally substituted C₃-C₈ cycloalkyl, optionally substituted aryl, or optionally substituted heteroaryl, or two R^{a} groups combine with the N to which they are bound to form a heterocycle.

Embodiment 4 provides the compound of any of Embodiments 1-3, which is:

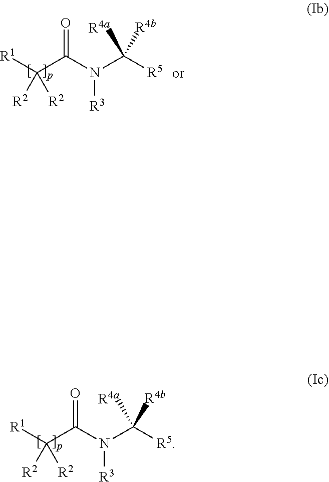

Embodiment 5 provides the compound of any of Embodiments 1-4, which is:

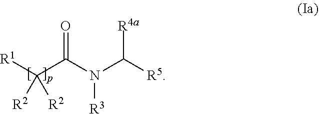

Embodiment 6 provides the compound of any of Embodiments 1-5, which is:

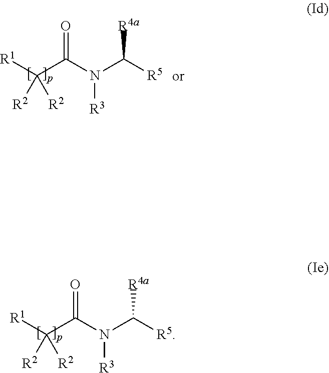

Embodiment 7 provides the compound of any of Embodiments 1-6, which is:

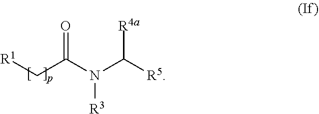

Embodiment 8 provides the compound of any of Embodiments 1-7, which:

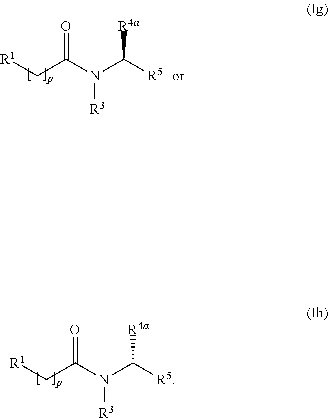

Embodiment 9 provides the compound of any of Embodiments 1-8, wherein R¹ is selected from the group consisting of:

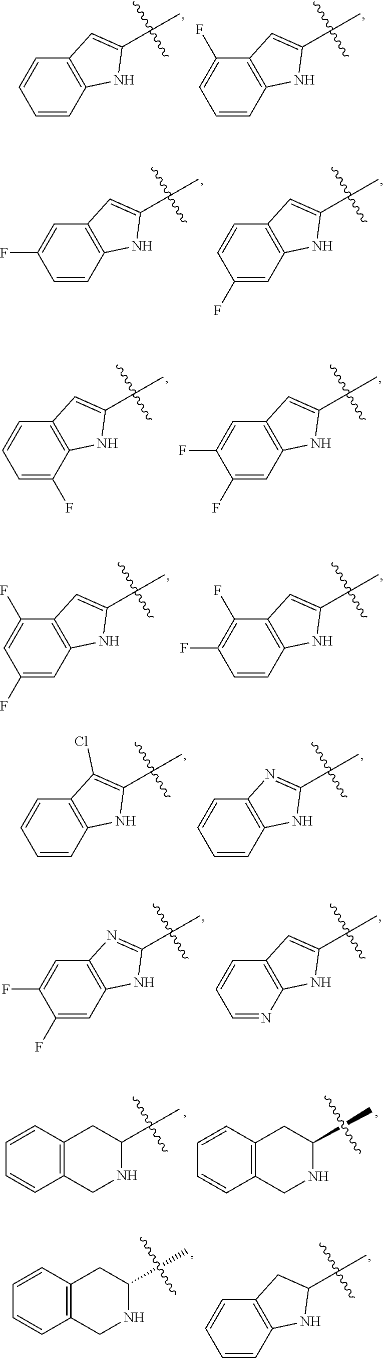

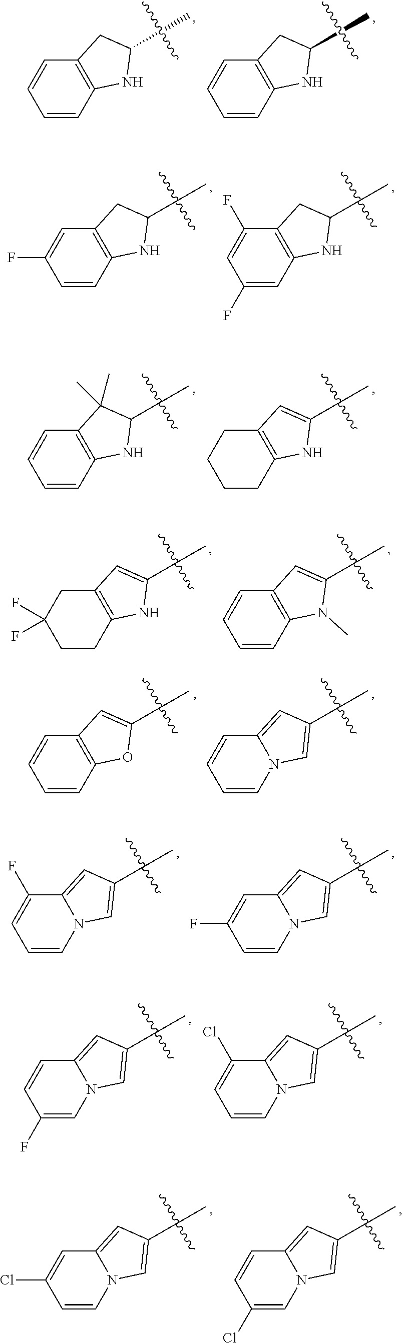

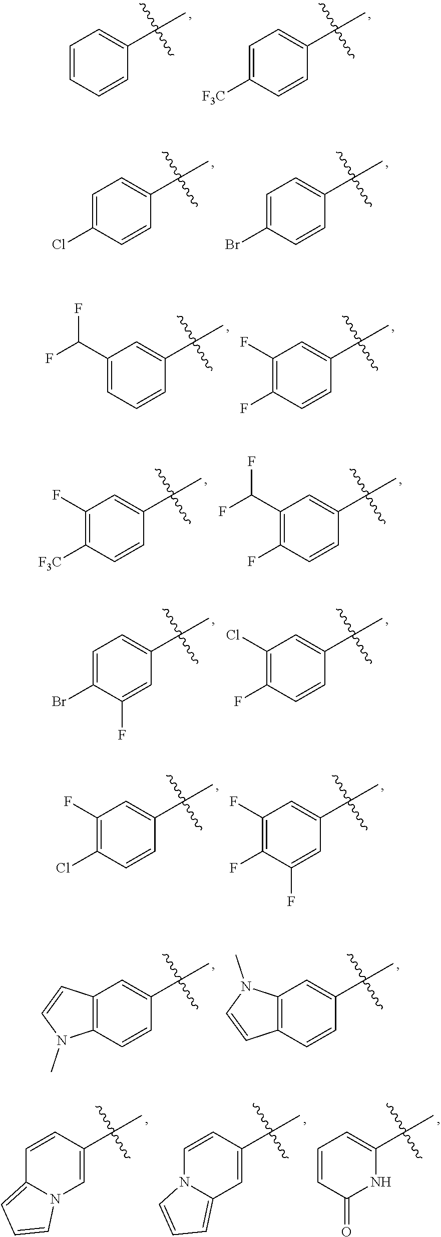

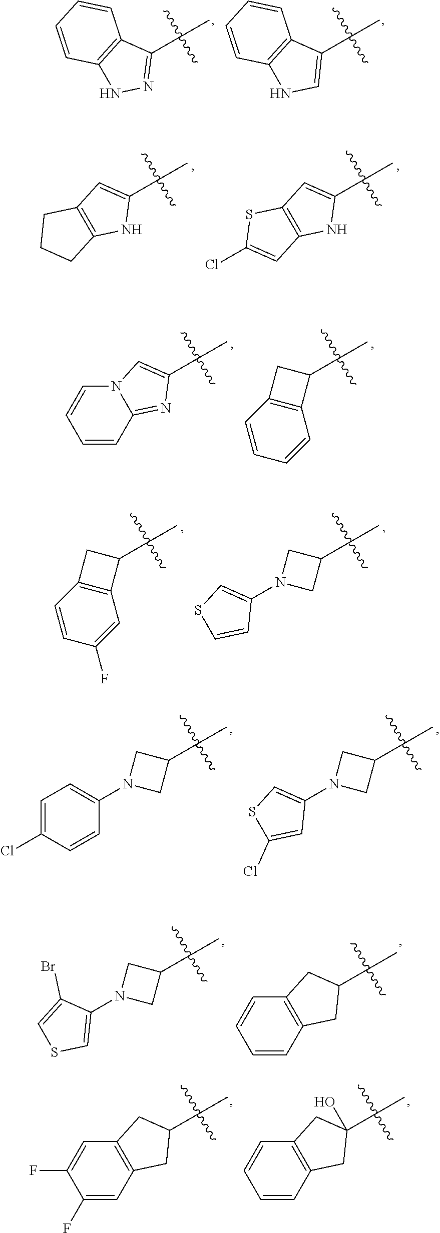

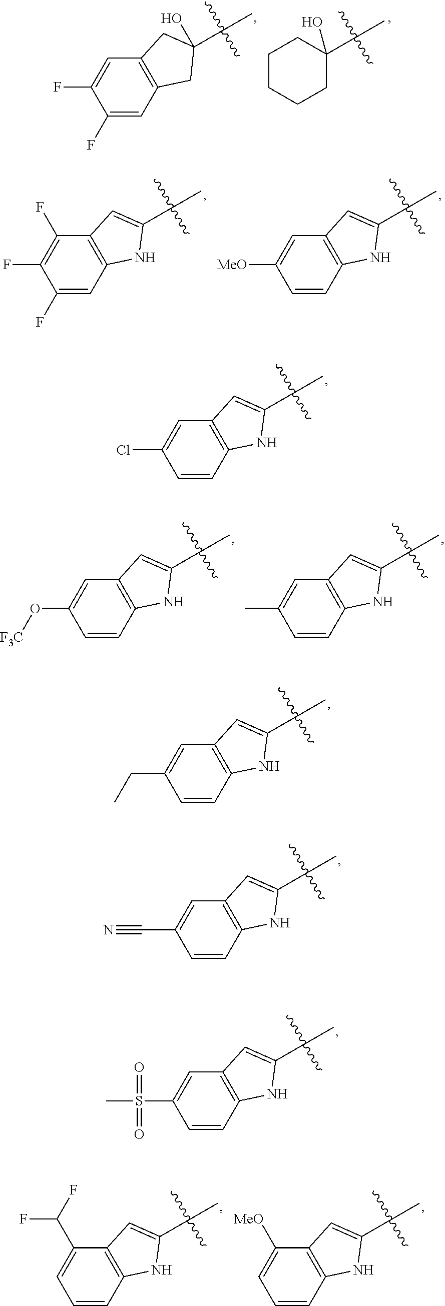

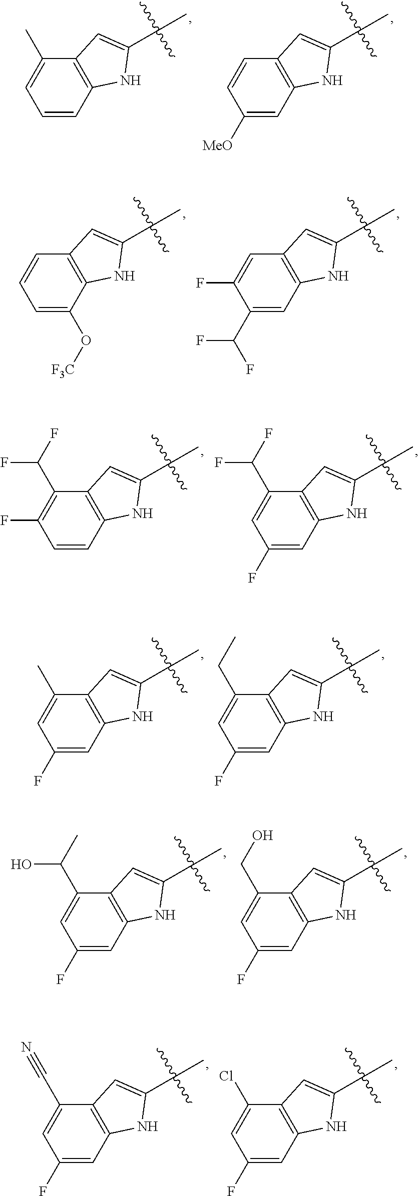

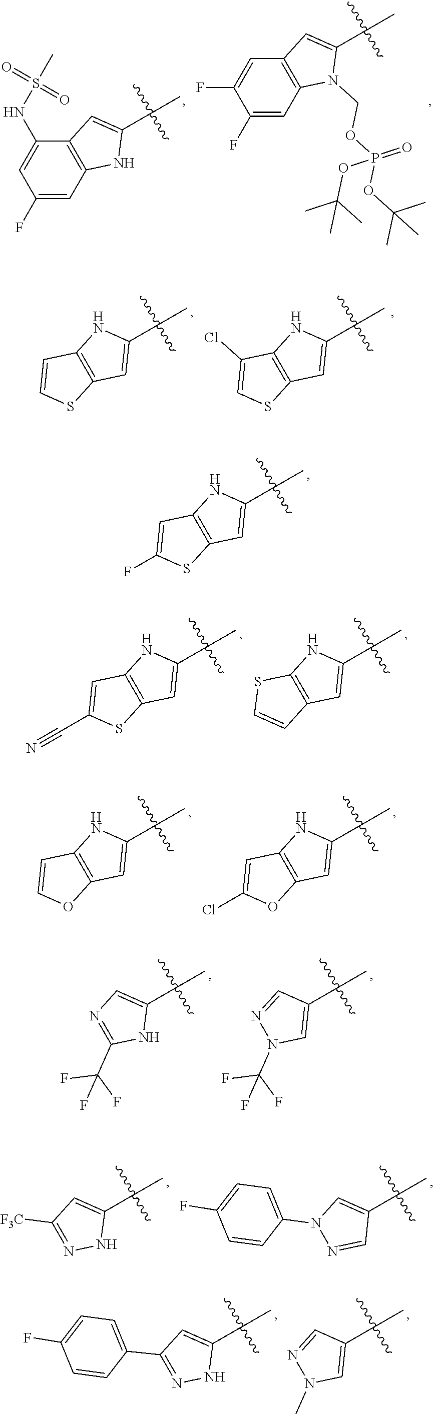

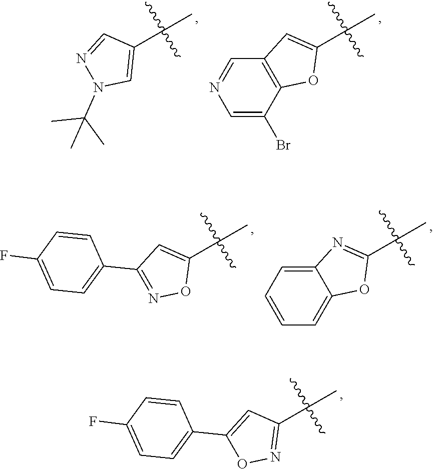

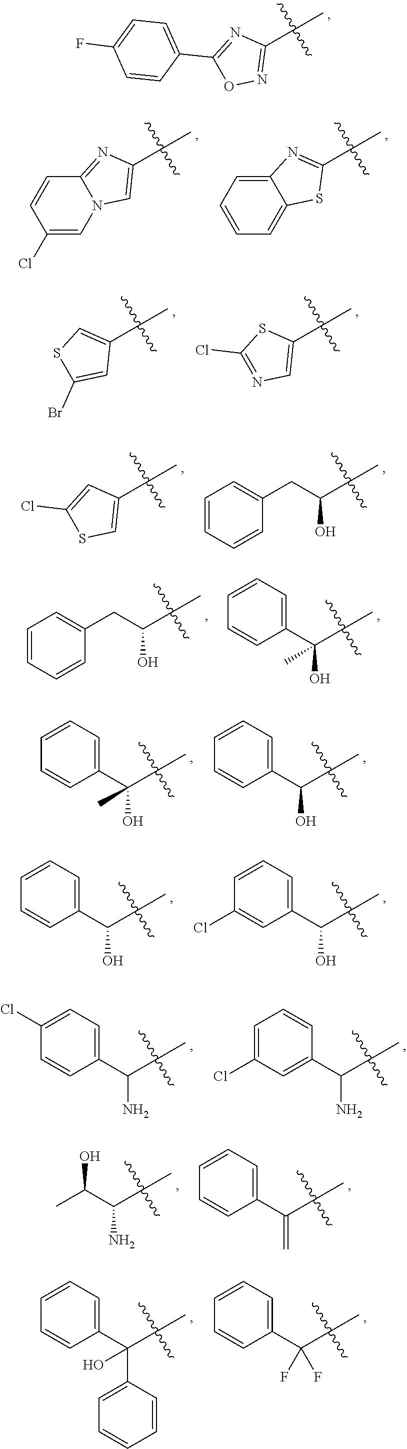

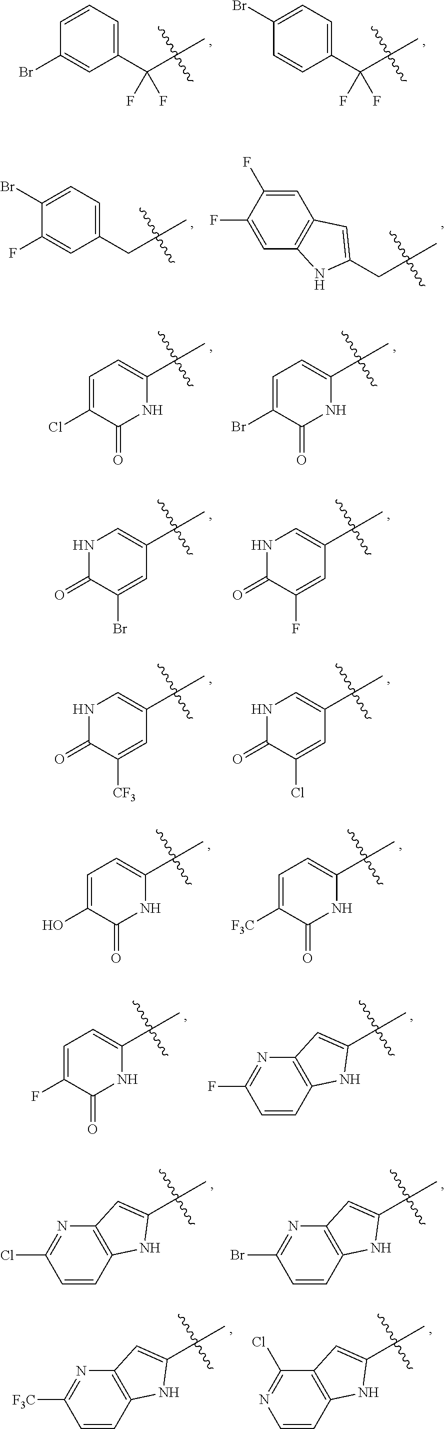

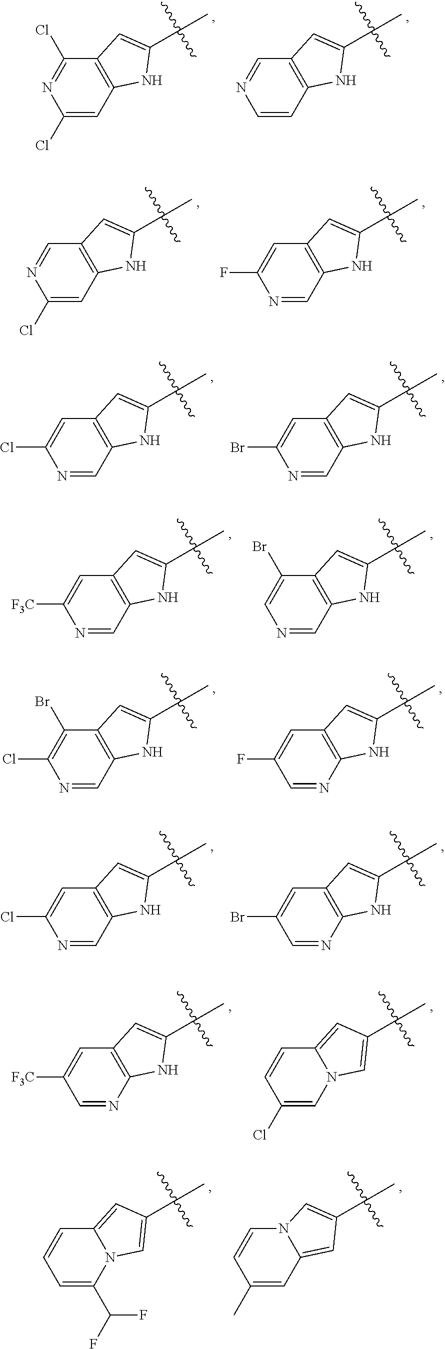

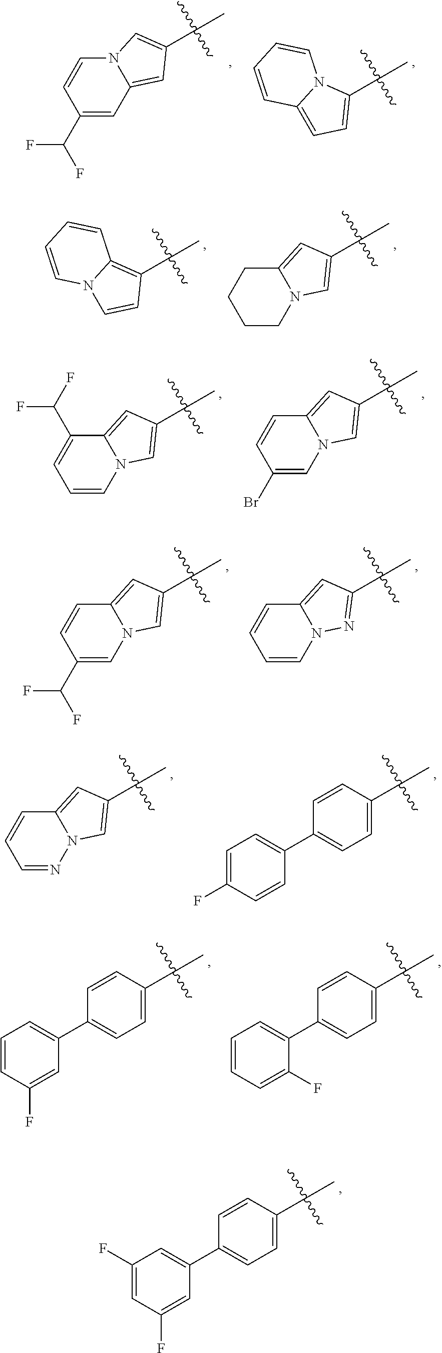

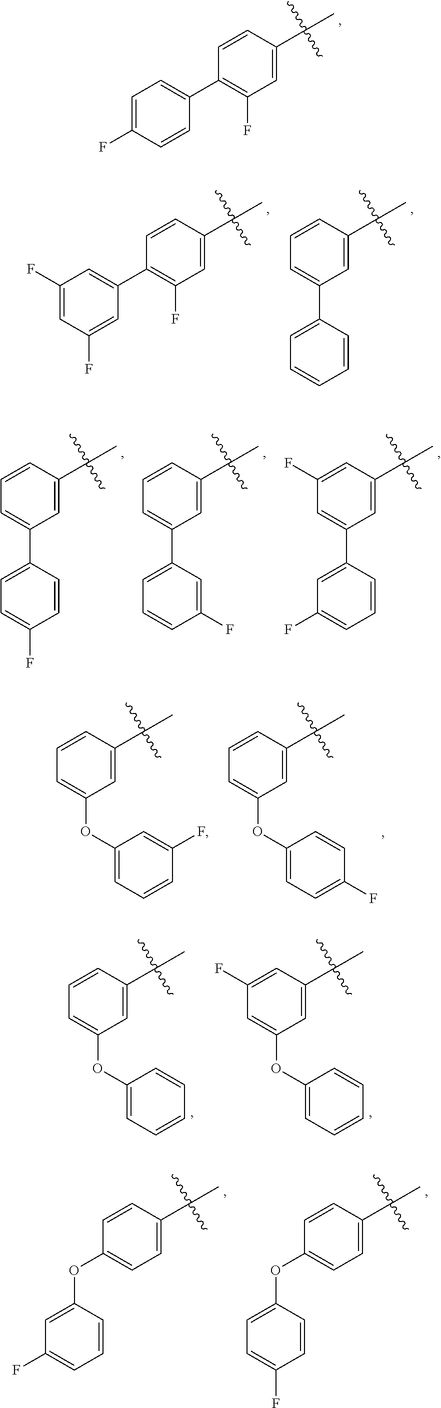

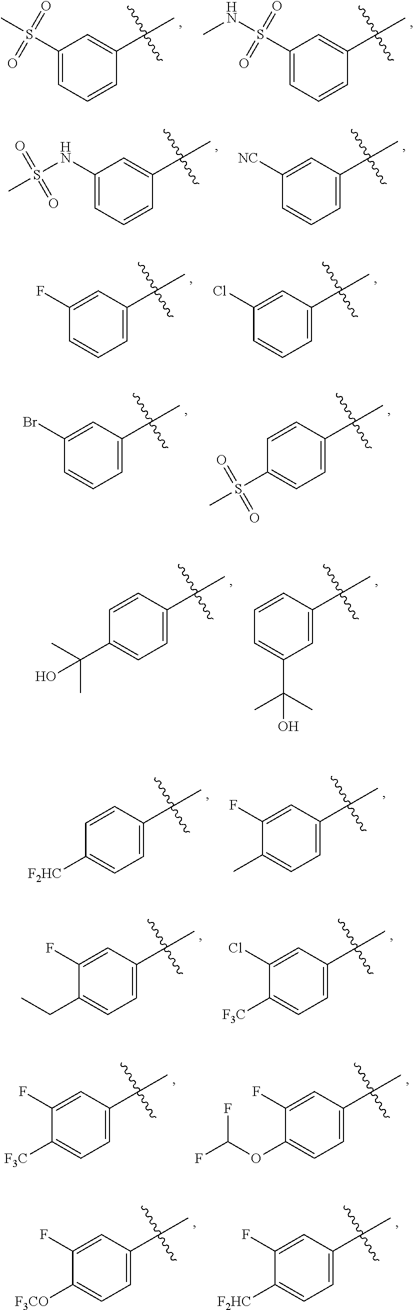

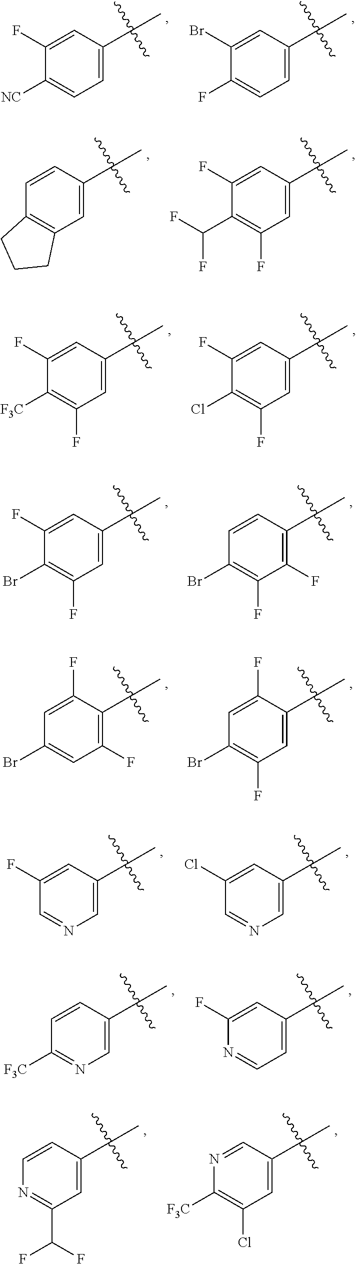

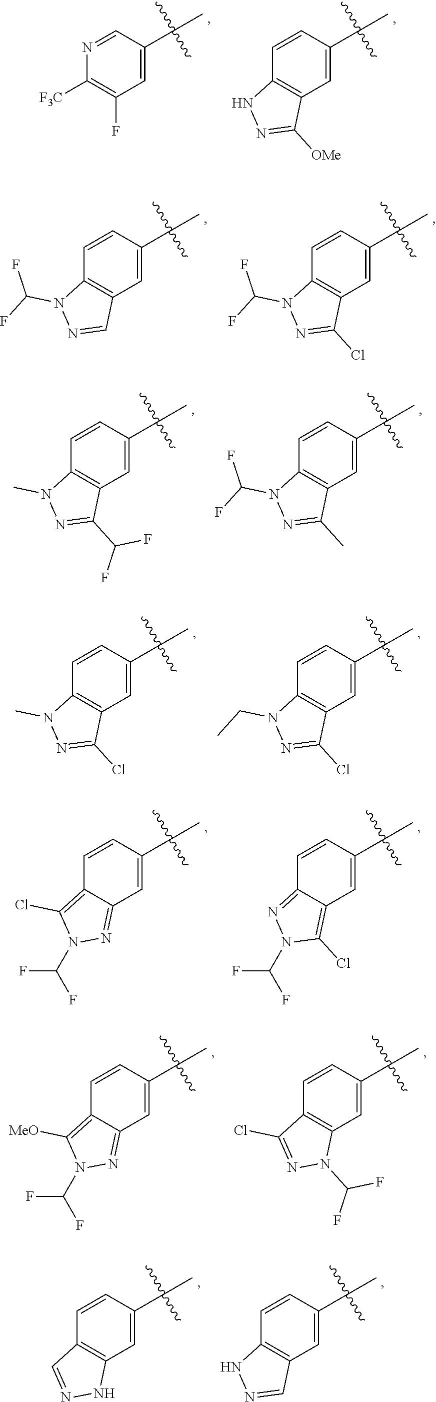

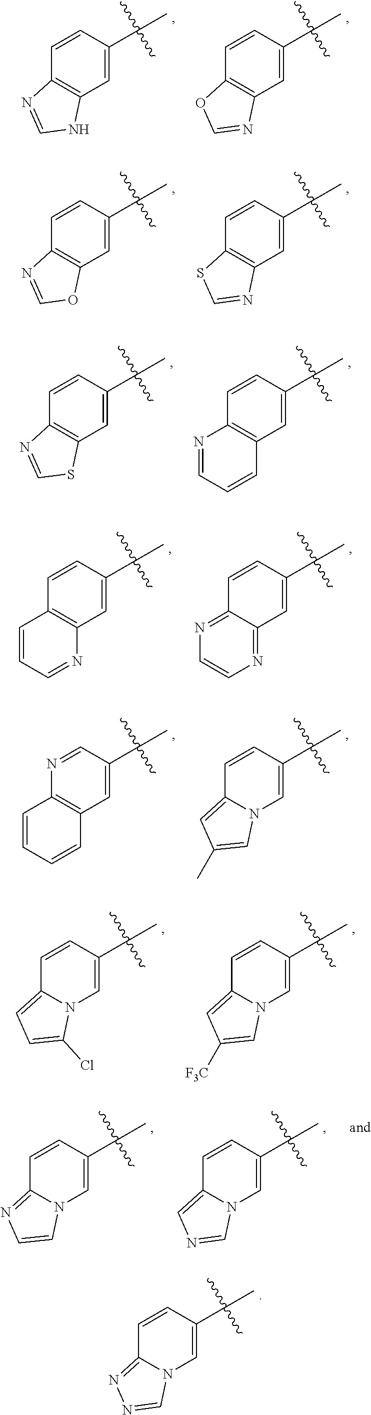

Embodiment 10 provides the compound of any of Embodiments 1-9, wherein each occurrence of R² is independently H or methyl.

Embodiment 11 provides the compound of any of Embodiments 1-10, wherein R³ is selected from the group consisting of H, methyl, ethyl, isopropyl, n-propyl, cyclopropyl, n-butyl, isobutyl, sec-butyl, tert-butyl, cyclobutyl, isopropylmethyl, —(CH₂)₂₋₆OH, —(CH₂)₂₋₆O(C₁-C₆ alkyl), optionally substituted benzyl, and optionally substituted phenyl.

Embodiment 12 provides the compound of any of Embodiments 1-11, wherein each of R^{4a} and R^{4b} is independently selected from the group consisting of H and methyl.

Embodiment 13 provides the compound of any of Embodiments 1-12, wherein R⁵ is

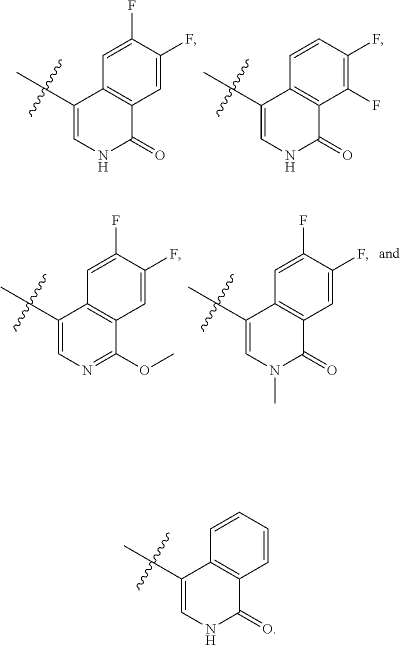 [SMILES: *c1c[nH]c(=O)c2cc(F)c(F)cc12 ; *c1c[nH]c(=O)c2c(F)c(F)ccc12 ; *c1cnc(OC)c2cc(F)c(F)cc12 ; *c1cn(C)c(=O)c2cc(F)c(F)cc12 ; *c1c[nH]c(=O)c2ccccc12]

Embodiment 14 provides the compound of any of Embodiments 1-13, wherein p is 0.

Embodiment 15 provides the compound of any of Embodiments 1-14, which is at least one selected from the group consisting of:
N-(1-(6,7-difluoro-1-oxo-1,2-dihydroisoquinolin-4-yl)ethyl)-N-methyl-1H-indole-2-carboxamide;
N-(1-(6,7-difluoro-1-oxo-1,2-dihydroisoquinolin-4-yl)ethyl)-N-isobutyl-1H-indole-2-carboxamide;
N-(1-(6,7-difluoro-1-oxo-1,2-dihydroisoquinolin-4-yl)ethyl)-N-ethyl-1H-indole-2-carboxamide;
N-(1-(6,7-difluoro-1-oxo-1,2-dihydroisoquinolin-4-yl)ethyl)-4-fluoro-N-methyl-1H-indole-2-carboxamide;
N-(1-(6,7-difluoro-1-oxo-1,2-dihydroisoquinolin-4-yl)ethyl)-5-fluoro-N-methyl-1H-indole-2-carboxamide;
N-(1-(6,7-difluoro-1-oxo-1,2-dihydroisoquinolin-4-yl)ethyl)-6-fluoro-N-methyl-1H-indole-2-carboxamide;
N-(1-(6,7-difluoro-1-oxo-1,2-dihydroisoquinolin-4-yl)ethyl)-7-fluoro-N-methyl-1H-indole-2-carboxamide;
N-(1-(6,7-difluoro-1-oxo-1,2-dihydroisoquinolin-4-yl)ethyl)-5,6-difluoro-N-methyl-1H-indole-2-carboxamide;
N-(1-(6,7-difluoro-1-oxo-1,2-dihydroisoquinolin-4-yl)ethyl)-4,6-difluoro-N-methyl-1H-indole-2-carboxamide;
N-(1-(6,7-difluoro-1-oxo-1,2-dihydroisoquinolin-4-yl)ethyl)-N-methyl-1,2,3,4-tetrahydroisoquinoline-3-carboxamide;
N-(1-(6,7-difluoro-1-oxo-1,2-dihydroisoquinolin-4-yl)ethyl)-N-methylindoline-2-carboxamide;
N-(1-(6,7-difluoro-1-oxo-1,2-dihydroisoquinolin-4-yl)ethyl)-N-methyl-1H-benzo[d]imidazole-2-carboxamide;
N-(1-(6,7-difluoro-1-oxo-1,2-dihydroisoquinolin-4-yl)ethyl)-N-methylbenzofuran-2-carboxamide;
N-(1-(6,7-difluoro-1-oxo-1,2-dihydroisoquinolin-4-yl)ethyl)-N-(3-hydroxypropyl)-1H-indole-2-carboxamide;
N-(1-(6,7-difluoro-1-oxo-1,2-dihydroisoquinolin-4-yl)ethyl)-4,5-difluoro-N-methyl-1H-indole-2-carboxamide;
N-(1-(6,7-difluoro-1-oxo-1,2-dihydroisoquinolin-4-yl)ethyl)-N-methylindoline-2-carboxamide;
N-(1-(6,7-difluoro-1-oxo-1,2-dihydroisoquinolin-4-yl)ethyl)-N-methyl-1,2,3,4-tetrahydroisoquinoline-3-carboxamide;
N-(1-(6,7-difluoro-1-oxo-1,2-dihydroisoquinolin-4-yl)ethyl)-N-methyl-4,5,6,7-tetrahydro-1H-indole-2-carboxamide;
N-(1-(6,7-difluoro-1-oxo-1,2-dihydroisoquinolin-4-yl)ethyl)-N,1-dimethyl-1H-indole-2-carboxamide;
N-(1-(6,7-difluoro-1-oxo-1,2-dihydroisoquinolin-4-yl)ethyl)-N-methylimidazo[1,2-a]pyridine-2-carboxamide;
N-(1-(6,7-difluoro-1-oxo-1,2-dihydroisoquinolin-4-yl)ethyl)-N-methyl-1,4,5,6-tetrahydrocyclopenta[b]pyrrole-2-carboxamide;
N-(1-(6,7-difluoro-1-oxo-1,2-dihydroisoquinolin-4-yl)ethyl)-5,6-difluoro-N-methyl-1H-benzo[d]imidazole-2-carboxamide;
N-(1-(6,7-difluoro-1-oxo-1,2-dihydroisoquinolin-4-yl)ethyl)-N-methyl-1H-pyrrolo[2,3-b]pyridine-2-carboxamide;
N-(1-(6,7-difluoro-1-oxo-1,2-dihydroisoquinolin-4-yl)ethyl)-N-methyl-1H-indole-3-carboxamide;
N-(1-(6,7-difluoro-1-oxo-1,2-dihydroisoquinolin-4-yl)ethyl)-N-methyl-1H-indazole-3-carboxamide;
N-(1-(7,8-difluoro-1-oxo-1,2-dihydroisoquinolin-4-yl)ethyl)-N-methyl-1,4,5,6-tetrahydrocyclopenta[b]pyrrole-2-carboxamide;
N-(1-(6,7-difluoro-1-oxo-1,2-dihydroisoquinolin-4-yl)ethyl)-2-(1H-indol-2-yl)-N-methylacetamide;
3-chloro-N-(1-(6,7-difluoro-1-oxo-1,2-dihydroisoquinolin-4-yl)ethyl)-N-methyl-1H-indole-2-carboxamide;
N-(1-(6,7-difluoro-1-oxo-1,2-dihydroisoquinolin-4-yl)ethyl)-5-fluoro-N-methylindoline-2-carboxamide;
N-(1-(7,8-difluoro-1-oxo-1,2-dihydroisoquinolin-4-yl)ethyl)-4,6-difluoro-N-methylindoline-2-carboxamide;
N-(1-(6,7-difluoro-1-oxo-1,2-dihydroisoquinolin-4-yl)ethyl)-N-methylindolizine-2-carboxamide;
N-(1-(7,8-difluoro-1-oxo-1,2-dihydroisoquinolin-4-yl)ethyl)-N-methylindolizine-2-carboxamide;
N-(1-(7,8-difluoro-1-oxo-1,2-dihydroisoquinolin-4-yl)ethyl)-5-fluoro-N-methylindoline-2-carboxamide;
8-chloro-N-(1-(6,7-difluoro-1-oxo-1,2-dihydroisoquinolin-4-yl)ethyl)-N-methylindolizine-2-carboxamide;
N-(1-(6,7-difluoro-1-oxo-1,2-dihydroisoquinolin-4-yl)ethyl)-8-fluoro-N-methylindolizine-2-carboxamide;
N-(1-(7,8-difluoro-1-oxo-1,2-dihydroisoquinolin-4-yl)ethyl)-8-fluoro-N-methylindolizine-2-carboxamide;
N-(1-(6,7-difluoro-1-oxo-1,2-dihydroisoquinolin-4-yl)ethyl)-N-(3-hydroxypropyl)-2-(1H-indol-2-yl)acetamide;
N-(1-(7,8-difluoro-1-oxo-1,2-dihydroisoquinolin-4-yl)ethyl)-N-methyl-6-oxo-1,6-dihydropyridine-2-carboxamide;
N-(1-(6,7-difluoro-1-oxo-1,2-dihydroisoquinolin-4-yl)ethyl)-5,5-difluoro-N-methyl-4,5,6,7-tetrahydro-1H-indole-2-carboxamide;
N-(1-(7,8-difluoro-1-oxo-1,2-dihydroisoquinolin-4-yl)ethyl)-5,5-difluoro-N-methyl-4,5,6,7-tetrahydro-1H-indole-2-carboxamide;
8-chloro-N-(1-(7,8-difluoro-1-oxo-1,2-dihydroisoquinolin-4-yl)ethyl)-N-methylindolizine-2-carboxamide;
3-chloro-N-(1-(6,7-difluoro-1-oxo-1,2-dihydroisoquinolin-4-yl)ethyl)-N-(3-hydroxypropyl)-1H-indole-2-carboxamide;
7-chloro-N-(1-(6,7-difluoro-1-oxo-1,2-dihydroisoquinolin-4-yl)ethyl)-N-methylindolizine-2-carboxamide;
N-(1-(6,7-difluoro-1-oxo-1,2-dihydroisoquinolin-4-yl)ethyl)-4,6-difluoro-N-methylindoline-2-carboxamide;
N-(1-(6,7-difluoro-1-oxo-1,2-dihydroisoquinolin-4-yl)ethyl)-6-fluoro-N-methylindolizine-2-carboxamide;
N-(1-(7,8-difluoro-1-oxo-1,2-dihydroisoquinolin-4-yl)ethyl)-N,3,3-trimethylindoline-2-carboxamide;
N-(1-(6,7-difluoro-1-oxo-1,2-dihydroisoquinolin-4-yl)ethyl)-7-fluoro-N-methylindolizine-2-carboxamide;
6-chloro-N-(1-(6,7-difluoro-1-oxo-1,2-dihydroisoquinolin-4-yl)ethyl)-N-methylindolizine-2-carboxamide;
N-(1-(6,7-difluoro-1-oxo-1,2-dihydroisoquinolin-4-yl)ethyl)-N,3,3-trimethylindoline-2-carboxamide;
N-(1-(6,7-Difluoro-1-oxo-1,2-dihydroisoquinolin-4-yl)ethyl)-N-isobutylindolizine-2-carboxamide;
N-(1-(6,7-difluoro-1-oxo-1,2-dihydroisoquinolin-4-yl)ethyl)-3-fluoro-N-isobutyl-4-(trifluoromethyl)benzamide;
N-(1-(6,7-Difluoro-1-oxo-1,2-dihydroisoquinolin-4-yl)ethyl)-3-fluoro-N-methyl-4-(trifluoromethyl) benzamide;
4-Chloro-N-(1-(6,7-difluoro-1-oxo-1,2-dihydroisoquinolin-4-yl)ethyl)-3-fluoro-N-methylbenzamide;
N-(1-(6,7-Difluoro-1-oxo-1,2-dihydroisoquinolin-4-yl)ethyl)-3,4,5-trifluoro-N-methylbenzamide;
N-(1-(6,7-Difluoro-1-oxo-1,2-dihydroisoquinolin-4-yl)ethyl)-3-(difluoromethyl)-N-methylbenzamide;
N-(1-(6,7-Difluoro-1-oxo-1,2-dihydroisoquinolin-4-yl)ethyl)-N-methylbenzamide
N-(1-(6,7-Difluoro-1-oxo-1,2-dihydroisoquinolin-4-yl)ethyl)-N-methyl-4-(trifluoromethyl) benzamide;
4-Chloro-N-(1-(6,7-difluoro-1-oxo-1,2-dihydroisoquinolin-4-yl)ethyl)-N-methylbenzamide;
N-(1-(6,7-Difluoro-1-oxo-1,2-dihydroisoquinolin-4-yl)ethyl)-3-(difluoromethyl)-4-fluoro-N-methylbenzamide;
3-Chloro-N-(1-(6,7-difluoro-1-oxo-1,2-dihydroisoquinolin-4-yl)ethyl)-4-fluoro-N-methylbenzamide;
N-(1-(6,7-Difluoro-1-oxo-1,2-dihydroisoquinolin-4-yl)ethyl)-3,4-difluoro-N-methylbenzamide;
N-(1-(6,7-Difluoro-1-oxo-1,2-dihydroisoquinolin-4-yl)ethyl)-N,1-dimethyl-1H-indole-5-carboxamide;
N-(1-(6,7-Difluoro-1-oxo-1,2-dihydroisoquinolin-4-yl)ethyl)-N,1-dimethyl-1H-indole-6-carboxamide;
N1-Cyclopropyl-N2-(1-(6,7-difluoro-1-oxo-1,2-dihydroisoquinolin-4-yl)ethyl)-N2-methyloxalamide;
N1-(3-Chloro-4-fluorophenyl)-N2-(1-(6,7-difluoro-1-oxo-1,2-dihydroisoquinolin-4-yl)ethyl)-N2-methyloxalamide;
N-(1-(7,8-difluoro-1-oxo-1,2-dihydroisoquinolin-4-yl)ethyl)-3-fluoro-N-isobutyl-4-(trifluoromethyl)benzamide;
3-fluoro-N-methyl-N-(1-(1-oxo-1,2-dihydroisoquinolin-4-yl)ethyl)-4-(trifluoromethyl)benzamide;
4-bromo-N-methyl-N-(1-(1-oxo-1,2-dihydroisoquinolin-4-yl)ethyl)benzamide;
N-methyl-N-(1-(1-oxo-1,2-dihydroisoquinolin-4-yl)ethyl)-4-(trifluoromethyl)benzamide;
4-chloro-3-fluoro-N-methyl-N-(1-(1-oxo-1,2-dihydroisoquinolin-4-yl)ethyl)benzamide;
3-chloro-4-fluoro-N-methyl-N-(1-(1-oxo-1,2-dihydroisoquinolin-4-yl)ethyl)benzamide;
4-bromo-3-fluoro-N-methyl-N-(1-(1-oxo-1,2-dihydroisoquinolin-4-yl)ethyl)benzamide;
2-(4-chlorophenyl)-N-methyl-N-(1-(1-oxo-1,2-dihydroisoquinolin-4-yl)ethyl)acetamide;
N-methyl-N-(1-(1-oxo-1,2-dihydroisoquinolin-4-yl)ethyl)indolizine-2-carboxamide;
7-fluoro-N-methyl-N-(1-(1-oxo-1,2-dihydroisoquinolin-4-yl)ethyl)indolizine-2-carboxamide;
8-chloro-N-methyl-N-(1-(1-oxo-1,2-dihydroisoquinolin-4-yl)ethyl)indolizine-2-carboxamide;
N-methyl-N-(1-(1-oxo-1,2-dihydroisoquinolin-4-yl)ethyl)-1H-indole-2-carboxamide;
4-fluoro-N-methyl-N-(1-(1-oxo-1,2-dihydroisoquinolin-4-yl)ethyl)-1H-indole-2-carboxamide;
5-fluoro-N-methyl-N-(1-(1-oxo-1,2-dihydroisoquinolin-4-yl)ethyl)-1H-indole-2-carboxamide;
4,6-difluoro-N-methyl-N-(1-(1-oxo-1,2-dihydroisoquinolin-4-yl)ethyl)-1H-indole-2-carboxamide;
5,6-difluoro-N-methyl-N-(1-(1-oxo-1,2-dihydroisoquinolin-4-yl)ethyl)-1H-indole-2-carboxamide;
N-methyl-N-((R)-1-(1-oxo-1,2-dihydroisoquinolin-4-yl)ethyl)indoline-2-carboxamide;
4-bromo-N-(1-(6,7-difluoro-2-methyl-1-oxo-1,2-dihydroisoquinolin-4-yl)ethyl)-3-fluoro-N-methylbenzamide;
4-bromo-N-(1-(6,7-difluoro-1-methoxyisoquinolin-4-yl)ethyl)-N-methylbenzamide;
N-(1-(6,7-difluoro-2-methyl-1-oxo-1,2-dihydroisoquinolin-4-yl)ethyl)-3-fluoro-N-methyl-4-(trifluoromethyl)benzamide;
4-bromo-N-(1-(6,7-difluoro-2-methyl-1-oxo-1,2-dihydroisoquinolin-4-yl)ethyl)-N-methylbenzamide;
N-(1-(6,7-difluoro-1-methoxyisoquinolin-4-yl)ethyl)-5,6-difluoro-N-methyl-1H-indole-2-carboxamide;
N-(1-(6,7-difluoro-2-methyl-1-oxo-1,2-dihydroisoquinolin-4-yl)ethyl)-8-fluoro-N-methylindolizine-2-carboxamide;
N-(1-(6,7-difluoro-2-methyl-1-oxo-1,2-dihydroisoquinolin-4-yl)ethyl)-5,6-difluoro-N-methyl-1H-indole-2-carboxamide;
N-(1-(6,7-difluoro-1-methoxyisoquinolin-4-yl)ethyl)-3-fluoro-N-methyl-4-(trifluoromethyl)benzamide;
4-bromo-N-(1-(6,7-difluoro-1-methoxyisoquinolin-4-yl)ethyl)-3-fluoro-N-methylbenzamide;
N-(1-(6,7-difluoro-1-methoxyisoquinolin-4-yl)ethyl)-8-fluoro-N-methylindolizine-2-carboxamide;
N-(1-(6,7-difluoro-1-methoxyisoquinolin-4-yl)ethyl)-N-methylindoline-2-carboxamide;
N-(1-(6,7-difluoro-2-methyl-1-oxo-1,2-dihydroisoquinolin-4-yl)ethyl)-N-methylindoline-2-carboxamide;
N-(1-(6,7-difluoro-1-oxo-1,2-dihydroisoquinolin-4-yl)ethyl)-N-methylindolizine-6-carboxamide;
2-chloro-N-(1-(6,7-difluoro-1-oxo-1,2-dihydroisoquinolin-4-yl)ethyl)-N-methyl-4H-thieno[3,2-b]pyrrole-5-carboxamide;
N-(1-(6,7-difluoro-1-oxo-1,2-dihydroisoquinolin-4-yl)ethyl)-N-methylindolizine-7-carboxamide;

or a salt, solvate, prodrug, isotopically labeled derivative, stereoisomer, or tautomer thereof, or any mixtures thereof.

Embodiment 16 provides the compound of any of Embodiments 1-15, which is at least one selected from the group consisting of:
(R)-N-(1-(6,7-difluoro-1-oxo-1,2-dihydroisoquinolin-4-yl)ethyl)-N-methyl-1H-indole-2-carboxamide;
(S)-N-(1-(6,7-difluoro-1-oxo-1,2-dihydroisoquinolin-4-yl)ethyl)-N-methyl-1H-indole-2-carboxamide;
(R)-N-(1-(6,7-difluoro-1-oxo-1,2-dihydroisoquinolin-4-yl)ethyl)-N-isobutyl-1H-indole-2-carboxamide;
(S)-N-(1-(6,7-difluoro-1-oxo-1,2-dihydroisoquinolin-4-yl)ethyl)-N-isobutyl-1H-indole-2-carboxamide;
(R)-N-(1-(6,7-difluoro-1-oxo-1,2-dihydroisoquinolin-4-yl)ethyl)-N-ethyl-1H-indole-2-carboxamide;
(S)-N-(1-(6,7-difluoro-1-oxo-1,2-dihydroisoquinolin-4-yl)ethyl)-N-ethyl-1H-indole-2-carboxamide;
(R)-N-(1-(6,7-difluoro-1-oxo-1,2-dihydroisoquinolin-4-yl)ethyl)-4-fluoro-N-methyl-1H-indole-2-carboxamide;
(S)-N-(1-(6,7-difluoro-1-oxo-1,2-dihydroisoquinolin-4-yl)ethyl)-4-fluoro-N-methyl-1H-indole-2-carboxamide;
(R)-N-(1-(6,7-difluoro-1-oxo-1,2-dihydroisoquinolin-4-yl)ethyl)-5-fluoro-N-methyl-1H-indole-2-carboxamide;
(S)-N-(1-(6,7-difluoro-1-oxo-1,2-dihydroisoquinolin-4-yl)ethyl)-5-fluoro-N-methyl-1H-indole-2-carboxamide;
(R)-N-(1-(6,7-difluoro-1-oxo-1,2-dihydroisoquinolin-4-yl)ethyl)-6-fluoro-N-methyl-1H-indole-2-carboxamide;
(S)-N-(1-(6,7-difluoro-1-oxo-1,2-dihydroisoquinolin-4-yl)ethyl)-6-fluoro-N-methyl-1H-indole-2-carboxamide;
(R)-N-(1-(6,7-difluoro-1-oxo-1,2-dihydroisoquinolin-4-yl)ethyl)-7-fluoro-N-methyl-1H-indole-2-carboxamide;
(S)-N-(1-(6,7-difluoro-1-oxo-1,2-dihydroisoquinolin-4-yl)ethyl)-7-fluoro-N-methyl-1H-indole-2-carboxamide;
(R)-N-(1-(6,7-difluoro-1-oxo-1,2-dihydroisoquinolin-4-yl)ethyl)-5,6-difluoro-N-methyl-1H-indole-2-carboxamide;
(S)-N-(1-(6,7-difluoro-1-oxo-1,2-dihydroisoquinolin-4-yl)ethyl)-5,6-difluoro-N-methyl-1H-indole-2-carboxamide;
(R)-N-(1-(6,7-difluoro-1-oxo-1,2-dihydroisoquinolin-4-yl)ethyl)-4,6-difluoro-N-methyl-1H-indole-2-carboxamide;
(S)-N-(1-(6,7-difluoro-1-oxo-1,2-dihydroisoquinolin-4-yl)ethyl)-4,6-difluoro-N-methyl-1H-indole-2-carboxamide;
(3R)-N-((1R)-(6,7-difluoro-1-oxo-1,2-dihydroisoquinolin-4-yl)ethyl)-N-methyl-1,2,3,4-tetrahydroisoquinoline-3-carboxamide;
(3R)-N-((1S)-(6,7-difluoro-1-oxo-1,2-dihydroisoquinolin-4-yl)ethyl)-N-methyl-1,2,3,4-tetrahydroisoquinoline-3-carboxamide;
(3 S)-N-((1R)-(6,7-difluoro-1-oxo-1,2-dihydroisoquinolin-4-yl)ethyl)-N-methyl-1,2,3,4-tetrahydroisoquinoline-3-carboxamide;
(3S)-N-((1S)-(6,7-difluoro-1-oxo-1,2-dihydroisoquinolin-4-yl)ethyl)-N-methyl-1,2,3,4-tetrahydroisoquinoline-3-carboxamide;
(2R)-N-((1R)-(6,7-difluoro-1-oxo-1,2-dihydroisoquinolin-4-yl)ethyl)-N-methylindoline-2-carboxamide;
(2R)-N-((1S)-(6,7-difluoro-1-oxo-1,2-dihydroisoquinolin-4-yl)ethyl)-N-methylindoline-2-carboxamide;
(2S)-N-((1R)-(6,7-difluoro-1-oxo-1,2-dihydroisoquinolin-4-yl)ethyl)-N-methylindoline-2-carboxamide;
(2S)-N-((1S)-(6,7-difluoro-1-oxo-1,2-dihydroisoquinolin-4-yl)ethyl)-N-methylindoline-2-carboxamide;
(R)-N-(1-(6,7-difluoro-1-oxo-1,2-dihydroisoquinolin-4-yl)ethyl)-N-methyl-1H-benzo[d]imidazole-2-carboxamide;
(S)-N-(1-(6,7-difluoro-1-oxo-1,2-dihydroisoquinolin-4-yl)ethyl)-N-methyl-1H-benzo[d]imidazole-2-carboxamide;
(R)-N-(1-(6,7-difluoro-1-oxo-1,2-dihydroisoquinolin-4-yl)ethyl)-N-methylbenzofuran-2-carboxamide;
(S)-N-(1-(6,7-difluoro-1-oxo-1,2-dihydroisoquinolin-4-yl)ethyl)-N-methylbenzofuran-2-carboxamide;
(R)-N-(1-(6,7-difluoro-1-oxo-1,2-dihydroisoquinolin-4-yl)ethyl)-N-(3-hydroxypropyl)-1H-indole-2-carboxamide;
(S)-N-(1-(6,7-difluoro-1-oxo-1,2-dihydroisoquinolin-4-yl)ethyl)-N-(3-hydroxypropyl)-1H-indole-2-carboxamide;
(R)-N-(1-(6,7-difluoro-1-oxo-1,2-dihydroisoquinolin-4-yl)ethyl)-4,5-difluoro-N-methyl-1H-indole-2-carboxamide;
(S)-N-(1-(6,7-difluoro-1-oxo-1,2-dihydroisoquinolin-4-yl)ethyl)-4,5-difluoro-N-methyl-1H-indole-2-carboxamide;
(2R)-N-((1R)-(6,7-difluoro-1-oxo-1,2-dihydroisoquinolin-4-yl)ethyl)-N-methylindoline-2-carboxamide;
(2R)-N-((1S)-(6,7-difluoro-1-oxo-1,2-dihydroisoquinolin-4-yl)ethyl)-N-methylindoline-2-carboxamide;
(2S)-N-((1R)-(6,7-difluoro-1-oxo-1,2-dihydroisoquinolin-4-yl)ethyl)-N-methylindoline-2-carboxamide;
(2S)-N-((1S)-(6,7-difluoro-1-oxo-1,2-dihydroisoquinolin-4-yl)ethyl)-N-methylindoline-2-carboxamide;
(3R)-N-((1R)-(6,7-difluoro-1-oxo-1,2-dihydroisoquinolin-4-yl)ethyl)-N-methyl-1,2,3,4-tetrahydroisoquinoline-3-carboxamide;
(3R)-N-((1S)-(6,7-difluoro-1-oxo-1,2-dihydroisoquinolin-4-yl)ethyl)-N-methyl-1,2,3,4-tetrahydroisoquinoline-3-carboxamide;
(3 S)-N-((1R)-(6,7-difluoro-1-oxo-1,2-dihydroisoquinolin-4-yl)ethyl)-N-methyl-1,2,3,4-tetrahydroisoquinoline-3-carboxamide;
(3 S)-N-((1S)-(6,7-difluoro-1-oxo-1,2-dihydroisoquinolin-4-yl)ethyl)-N-methyl-1,2,3,4-tetrahydroisoquinoline-3-carboxamide;
(R)-N-(1-(6,7-difluoro-1-oxo-1,2-dihydroisoquinolin-4-yl)ethyl)-N-methyl-4,5,6,7-tetrahydro-1H-indole-2-carboxamide;
(S)-N-(1-(6,7-difluoro-1-oxo-1,2-dihydroisoquinolin-4-yl)ethyl)-N-methyl-4,5,6,7-tetrahydro-1H-indole-2-carboxamide;
(R)-N-(1-(6,7-difluoro-1-oxo-1,2-dihydroisoquinolin-4-yl)ethyl)-N,1-dimethyl-1H-indole-2-carboxamide;
(S)-N-(1-(6,7-difluoro-1-oxo-1,2-dihydroisoquinolin-4-yl)ethyl)-N,1-dimethyl-1H-indole-2-carboxamide;
(R)-N-(1-(6,7-difluoro-1-oxo-1,2-dihydroisoquinolin-4-yl)ethyl)-N-methylimidazo[1,2-a]pyridine-2-carboxamide;
(S)-N-(1-(6,7-difluoro-1-oxo-1,2-dihydroisoquinolin-4-yl)ethyl)-N-methylimidazo[1,2-a]pyridine-2-carboxamide;
(R)-N-(1-(6,7-difluoro-1-oxo-1,2-dihydroisoquinolin-4-yl)ethyl)-N-methyl-1,4,5,6-tetrahydrocyclopenta[b]pyrrole-2-carboxamide;
(S)-N-(1-(6,7-difluoro-1-oxo-1,2-dihydroisoquinolin-4-yl)ethyl)-N-methyl-1,4,5,6-tetrahydrocyclopenta[b]pyrrole-2-carboxamide;
(R)-N-(1-(6,7-difluoro-1-oxo-1,2-dihydroisoquinolin-4-yl)ethyl)-5,6-difluoro-N-methyl-1H-benzo[d]imidazole-2-carboxamide;
(S)-N-(1-(6,7-difluoro-1-oxo-1,2-dihydroisoquinolin-4-yl)ethyl)-5,6-difluoro-N-methyl-1H-benzo[d]imidazole-2-carboxamide;
(R)-N-(1-(6,7-difluoro-1-oxo-1,2-dihydroisoquinolin-4-yl)ethyl)-N-methyl-1H-pyrrolo[2,3-b]pyridine-2-carboxamide;
(S)-N-(1-(6,7-difluoro-1-oxo-1,2-dihydroisoquinolin-4-yl)ethyl)-N-methyl-1H-pyrrolo[2,3-b]pyridine-2-carboxamide;
(R)-N-(1-(6,7-difluoro-1-oxo-1,2-dihydroisoquinolin-4-yl)ethyl)-N-methyl-1H-indole-3-carboxamide;
(S)-N-(1-(6,7-difluoro-1-oxo-1,2-dihydroisoquinolin-4-yl)ethyl)-N-methyl-1H-indole-3-carboxamide;
(R)-N-(1-(6,7-difluoro-1-oxo-1,2-dihydroisoquinolin-4-yl)ethyl)-N-methyl-1H-indazole-3-carboxamide;
(S)-N-(1-(6,7-difluoro-1-oxo-1,2-dihydroisoquinolin-4-yl)ethyl)-N-methyl-1H-indazole-3-carboxamide;
(R)-N-(1-(7,8-difluoro-1-oxo-1,2-dihydroisoquinolin-4-yl)ethyl)-N-methyl-1,4,5,6-tetrahydrocyclopenta[b]pyrrole-2-carboxamide;
(S)-N-(1-(7,8-difluoro-1-oxo-1,2-dihydroisoquinolin-4-yl)ethyl)-N-methyl-1,4,5,6-tetrahydrocyclopenta[b]pyrrole-2-carboxamide;
(R)-N-(1-(6,7-difluoro-1-oxo-1,2-dihydroisoquinolin-4-yl)ethyl)-2-(1H-indol-2-yl)-N-methylacetamide;
(S)-N-(1-(6,7-difluoro-1-oxo-1,2-dihydroisoquinolin-4-yl)ethyl)-2-(1H-indol-2-yl)-N-methylacetamide;
(R)-3-chloro-N-(1-(6,7-difluoro-1-oxo-1,2-dihydroisoquinolin-4-yl)ethyl)-N-methyl-1H-indole-2-carboxamide;
(S)-3-chloro-N-(1-(6,7-difluoro-1-oxo-1,2-dihydroisoquinolin-4-yl)ethyl)-N-methyl-1H-indole-2-carboxamide;
N-((1R)-(6,7-difluoro-1-oxo-1,2-dihydroisoquinolin-4-yl)ethyl)-5-fluoro-N-methylindoline-(2R)-carboxamide;
N-((1R)-(6,7-difluoro-1-oxo-1,2-dihydroisoquinolin-4-yl)ethyl)-5-fluoro-N-methylindoline-(2S)-carboxamide;
N-((1S)-(6,7-difluoro-1-oxo-1,2-dihydroisoquinolin-4-yl)ethyl)-5-fluoro-N-methylindoline-(2R)-carboxamide;
N-((1S)-(6,7-difluoro-1-oxo-1,2-dihydroisoquinolin-4-yl)ethyl)-5-fluoro-N-methylindoline-(2S)-carboxamide;
N-((1R)-(7,8-difluoro-1-oxo-1,2-dihydroisoquinolin-4-yl)ethyl)-4,6-difluoro-N-methylindoline-(2R)-carboxamide;
N-((1R)-(7,8-difluoro-1-oxo-1,2-dihydroisoquinolin-4-yl)ethyl)-4,6-difluoro-N-methylindoline-(2S)-carboxamide;
N-((1S)-(7,8-difluoro-1-oxo-1,2-dihydroisoquinolin-4-yl)ethyl)-4,6-difluoro-N-methylindoline-(2R)-carboxamide;
N-((1S)-(7,8-difluoro-1-oxo-1,2-dihydroisoquinolin-4-yl)ethyl)-4,6-difluoro-N-methylindoline-(2S)-carboxamide;
(R)-N-(1-(6,7-difluoro-1-oxo-1,2-dihydroisoquinolin-4-yl)ethyl)-N-methylindolizine-2-carboxamide;
(S)-N-(1-(6,7-difluoro-1-oxo-1,2-dihydroisoquinolin-4-yl)ethyl)-N-methylindolizine-2-carboxamide;
(R)-N-(1-(7,8-difluoro-1-oxo-1,2-dihydroisoquinolin-4-yl)ethyl)-N-methylindolizine-2-carboxamide;
(S)-N-(1-(7,8-difluoro-1-oxo-1,2-dihydroisoquinolin-4-yl)ethyl)-N-methylindolizine-2-carboxamide;
N-((1R)-(7,8-difluoro-1-oxo-1,2-dihydroisoquinolin-4-yl)ethyl)-5-fluoro-N-methylindoline-(2R)-carboxamide;
N-((1R)-(7,8-difluoro-1-oxo-1,2-dihydroisoquinolin-4-yl)ethyl)-5-fluoro-N-methylindoline-(2S)-carboxamide;
N-((1S)-(7,8-difluoro-1-oxo-1,2-dihydroisoquinolin-4-yl)ethyl)-5-fluoro-N-methylindoline-(2R)-carboxamide;
N-((1S)-(7,8-difluoro-1-oxo-1,2-dihydroisoquinolin-4-yl)ethyl)-5-fluoro-N-methylindoline-(2S)-carboxamide;
(R)-8-chloro-N-(1-(6,7-difluoro-1-oxo-1,2-dihydroisoquinolin-4-yl)ethyl)-N-methylindolizine-2-carboxamide;
(S)-8-chloro-N-(1-(6,7-difluoro-1-oxo-1,2-dihydroisoquinolin-4-yl)ethyl)-N-methylindolizine-2-carboxamide;
(R)-N-(1-(6,7-difluoro-1-oxo-1,2-dihydroisoquinolin-4-yl)ethyl)-8-fluoro-N-methylindolizine-2-carboxamide;
(S)-N-(1-(6,7-difluoro-1-oxo-1,2-dihydroisoquinolin-4-yl)ethyl)-8-fluoro-N-methylindolizine-2-carboxamide;
(R)-N-(1-(7,8-difluoro-1-oxo-1,2-dihydroisoquinolin-4-yl)ethyl)-8-fluoro-N-methylindolizine-2-carboxamide;
(S)-N-(1-(7,8-difluoro-1-oxo-1,2-dihydroisoquinolin-4-yl)ethyl)-8-fluoro-N-methylindolizine-2-carboxamide;
(R)-N-(1-(6,7-difluoro-1-oxo-1,2-dihydroisoquinolin-4-yl)ethyl)-N-(3-hydroxypropyl)-2-(1H-indol-2-yl)acetamide;
(S)-N-(1-(6,7-difluoro-1-oxo-1,2-dihydroisoquinolin-4-yl)ethyl)-N-(3-hydroxypropyl)-2-(1H-indol-2-yl)acetamide;
(R)-N-(1-(7,8-difluoro-1-oxo-1,2-dihydroisoquinolin-4-yl)ethyl)-N-methyl-6-oxo-1,6-dihydropyridine-2-carboxamide;
(S)-N-(1-(7,8-difluoro-1-oxo-1,2-dihydroisoquinolin-4-yl)ethyl)-N-methyl-6-oxo-1,6-dihydropyridine-2-carboxamide;
(R)-N-(1-(6,7-difluoro-1-oxo-1,2-dihydroisoquinolin-4-yl)ethyl)-5,5-difluoro-N-methyl-4,5,6,7-tetrahydro-1H-indole-2-carboxamide;
(S)-N-(1-(6,7-difluoro-1-oxo-1,2-dihydroisoquinolin-4-yl)ethyl)-5,5-difluoro-N-methyl-4,5,6,7-tetrahydro-1H-indole-2-carboxamide;
(R)-N-(1-(7,8-difluoro-1-oxo-1,2-dihydroisoquinolin-4-yl)ethyl)-5,5-difluoro-N-methyl-4,5,6,7-tetrahydro-1H-indole-2-carboxamide;
(S)-N-(1-(7,8-difluoro-1-oxo-1,2-dihydroisoquinolin-4-yl)ethyl)-5,5-difluoro-N-methyl-4,5,6,7-tetrahydro-1H-indole-2-carboxamide;
(R)-8-chloro-N-(1-(7,8-difluoro-1-oxo-1,2-dihydroisoquinolin-4-yl)ethyl)-N-methylindolizine-2-carboxamide;
(S)-8-chloro-N-(1-(7,8-difluoro-1-oxo-1,2-dihydroisoquinolin-4-yl)ethyl)-N-methylindolizine-2-carboxamide;
(R)-3-chloro-N-(1-(6,7-difluoro-1-oxo-1,2-dihydroisoquinolin-4-yl)ethyl)-N-(3-hydroxypropyl)-1H-indole-2-carboxamide;
(S)-3-chloro-N-(1-(6,7-difluoro-1-oxo-1,2-dihydroisoquinolin-4-yl)ethyl)-N-(3-hydroxypropyl)-1H-indole-2-carboxamide;
(R)-7-chloro-N-(1-(6,7-difluoro-1-oxo-1,2-dihydroisoquinolin-4-yl)ethyl)-N-methylindolizine-2-carboxamide;
(S)-7-chloro-N-(1-(6,7-difluoro-1-oxo-1,2-dihydroisoquinolin-4-yl)ethyl)-N-methylindolizine-2-carboxamide;
N-((1R)-(6,7-difluoro-1-oxo-1,2-dihydroisoquinolin-4-yl)ethyl)-4,6-difluoro-N-methylindoline-(2R)-carboxamide;
N-((1R)-(6,7-difluoro-1-oxo-1,2-dihydroisoquinolin-4-yl)ethyl)-4,6-difluoro-N-methylindoline-(2S)-carboxamide;
N-((1S)-(6,7-difluoro-1-oxo-1,2-dihydroisoquinolin-4-yl)ethyl)-4,6-difluoro-N-methylindoline-(2R)-carboxamide;
N-((1S)-(6,7-difluoro-1-oxo-1,2-dihydroisoquinolin-4-yl)ethyl)-4,6-difluoro-N-methylindoline-(2S)-carboxamide;
(R)-N-(1-(6,7-difluoro-1-oxo-1,2-dihydroisoquinolin-4-yl)ethyl)-6-fluoro-N-methylindolizine-2-carboxamide;
(S)-N-(1-(6,7-difluoro-1-oxo-1,2-dihydroisoquinolin-4-yl)ethyl)-6-fluoro-N-methylindolizine-2-carboxamide;
N-((1R)-(7,8-difluoro-1-oxo-1,2-dihydroisoquinolin-4-yl)ethyl)-N,3,3-trimethylindoline-(2R)-carboxamide;
N-((1R)-(7,8-difluoro-1-oxo-1,2-dihydroisoquinolin-4-yl)ethyl)-N,3,3-trimethylindoline-(2S)-carboxamide;
N-((1S)-(7,8-difluoro-1-oxo-1,2-dihydroisoquinolin-4-yl)ethyl)-N,3,3-trimethylindoline-(2R)-carboxamide;
N-((1S)-(7,8-difluoro-1-oxo-1,2-dihydroisoquinolin-4-yl)ethyl)-N,3,3-trimethylindoline-(2S)-carboxamide;
(R)-N-(1-(6,7-difluoro-1-oxo-1,2-dihydroisoquinolin-4-yl)ethyl)-7-fluoro-N-methylindolizine-2-carboxamide;
(S)-N-(1-(6,7-difluoro-1-oxo-1,2-dihydroisoquinolin-4-yl)ethyl)-7-fluoro-N-methylindolizine-2-carboxamide;
(R)-6-chloro-N-(1-(6,7-difluoro-1-oxo-1,2-dihydroisoquinolin-4-yl)ethyl)-N-methylindolizine-2-carboxamide;
(S)-6-chloro-N-(1-(6,7-difluoro-1-oxo-1,2-dihydroisoquinolin-4-yl)ethyl)-N-methylindolizine-2-carboxamide;
N-((1R)-(6,7-difluoro-1-oxo-1,2-dihydroisoquinolin-4-yl)ethyl)-N,3,3-trimethylindoline-(2R)-carboxamide;
N-((1R)-(6,7-difluoro-1-oxo-1,2-dihydroisoquinolin-4-yl)ethyl)-N,3,3-trimethylindoline-(2S)-carboxamide;
N-((1S)-(6,7-difluoro-1-oxo-1,2-dihydroisoquinolin-4-yl)ethyl)-N,3,3-trimethylindoline-(2R)-carboxamide;
N-((1S)-(6,7-difluoro-1-oxo-1,2-dihydroisoquinolin-4-yl)ethyl)-N,3,3-trimethylindoline-(2S)-carboxamide;
(R)-N-(1-(6,7-Difluoro-1-oxo-1,2-dihydroisoquinolin-4-yl)ethyl)-N-isobutylindolizine-2-carboxamide;
(S)-N-(1-(6,7-Difluoro-1-oxo-1,2-dihydroisoquinolin-4-yl)ethyl)-N-isobutylindolizine-2-carboxamide;
(R)-N-(1-(6,7-difluoro-1-oxo-1,2-dihydroisoquinolin-4-yl)ethyl)-3-fluoro-N-isobutyl-4-(trifluoromethyl)benzamide;
(S)-N-(1-(6,7-difluoro-1-oxo-1,2-dihydroisoquinolin-4-yl)ethyl)-3-fluoro-N-isobutyl-4-(trifluoromethyl)benzamide;
(R)-N-(1-(6,7-Difluoro-1-oxo-1,2-dihydroisoquinolin-4-yl)ethyl)-3-fluoro-N-methyl-4-(trifluoromethyl) benzamide;
(S)-N-(1-(6,7-Difluoro-1-oxo-1,2-dihydroisoquinolin-4-yl)ethyl)-3-fluoro-N-methyl-4-(trifluoromethyl) benzamide;
(R)-4-Chloro-N-(1-(6,7-difluoro-1-oxo-1,2-dihydroisoquinolin-4-yl)ethyl)-3-fluoro-N-methylbenzamide;
(S)-4-Chloro-N-(1-(6,7-difluoro-1-oxo-1,2-dihydroisoquinolin-4-yl)ethyl)-3-fluoro-N-methylbenzamide;
(R)-N-(1-(6,7-Difluoro-1-oxo-1,2-dihydroisoquinolin-4-yl)ethyl)-3,4,5-trifluoro-N-methylbenzamide;
(S)-N-(1-(6,7-Difluoro-1-oxo-1,2-dihydroisoquinolin-4-yl)ethyl)-3,4,5-trifluoro-N-methylbenzamide;
(R)-N-(1-(6,7-Difluoro-1-oxo-1,2-dihydroisoquinolin-4-yl)ethyl)-3-(difluoromethyl)-N-methylbenzamide;
(S)-N-(1-(6,7-Difluoro-1-oxo-1,2-dihydroisoquinolin-4-yl)ethyl)-3-(difluoromethyl)-N-methylbenzamide;
(R)-N-(1-(6,7-Difluoro-1-oxo-1,2-dihydroisoquinolin-4-yl)ethyl)-N-methylbenzamide;
(S)-N-(1-(6,7-Difluoro-1-oxo-1,2-dihydroisoquinolin-4-yl)ethyl)-N-methylbenzamide;
(R)-N-(1-(6,7-Difluoro-1-oxo-1,2-dihydroisoquinolin-4-yl)ethyl)-N-methyl-4-(trifluoromethyl) benzamide;
(S)-N-(1-(6,7-Difluoro-1-oxo-1,2-dihydroisoquinolin-4-yl)ethyl)-N-methyl-4-(trifluoromethyl) benzamide;
(R)-4-Chloro-N-(1-(6,7-difluoro-1-oxo-1,2-dihydroisoquinolin-4-yl)ethyl)-N-methylbenzamide;
(S)-4-Chloro-N-(1-(6,7-difluoro-1-oxo-1,2-dihydroisoquinolin-4-yl)ethyl)-N-methylbenzamide;
(R)-N-(1-(6,7-Difluoro-1-oxo-1,2-dihydroisoquinolin-4-yl)ethyl)-3-(difluoromethyl)-4-fluoro-N-methylbenzamide;
(S)-N-(1-(6,7-Difluoro-1-oxo-1,2-dihydroisoquinolin-4-yl)ethyl)-3-(difluoromethyl)-4-fluoro-N-methylbenzamide;
(R)-3-Chloro-N-(1-(6,7-difluoro-1-oxo-1,2-dihydroisoquinolin-4-yl)ethyl)-4-fluoro-N-methylbenzamide;
(S)-3-Chloro-N-(1-(6,7-difluoro-1-oxo-1,2-dihydroisoquinolin-4-yl)ethyl)-4-fluoro-N-methylbenzamide;
(R)-N-(1-(6,7-Difluoro-1-oxo-1,2-dihydroisoquinolin-4-yl)ethyl)-3,4-difluoro-N-methylbenzamide;
(S)-N-(1-(6,7-Difluoro-1-oxo-1,2-dihydroisoquinolin-4-yl)ethyl)-3,4-difluoro-N-methylbenzamide;
(R)-N-(1-(6,7-Difluoro-1-oxo-1,2-dihydroisoquinolin-4-yl)ethyl)-N,1-dimethyl-1H-indole-5-carboxamide;
(S)-N-(1-(6,7-Difluoro-1-oxo-1,2-dihydroisoquinolin-4-yl)ethyl)-N,1-dimethyl-1H-indole-5-carboxamide;
(R)-N-(1-(6,7-Difluoro-1-oxo-1,2-dihydroisoquinolin-4-yl)ethyl)-N,1-dimethyl-1H-indole-6-carboxamide;
(S)-N-(1-(6,7-Difluoro-1-oxo-1,2-dihydroisoquinolin-4-yl)ethyl)-N,1-dimethyl-1H-indole-6-carboxamide;
(R)-N1-Cyclopropyl-N2-(1-(6,7-difluoro-1-oxo-1,2-dihydroisoquinolin-4-yl)ethyl)-N2-methyloxalamide;
(S)-N1-Cyclopropyl-N2-(1-(6,7-difluoro-1-oxo-1,2-dihydroisoquinolin-4-yl)ethyl)-N2-methyloxalamide;
(R)-N1-(3-Chloro-4-fluorophenyl)-N2-(1-(6,7-difluoro-1-oxo-1,2-dihydroisoquinolin-4-yl)ethyl)-N2-methyloxalamide;
(S)-N1-(3-Chloro-4-fluorophenyl)-N2-(1-(6,7-difluoro-1-oxo-1,2-dihydroisoquinolin-4-yl)ethyl)-N2-methyloxalamide;
(R)-N-(1-(7,8-difluoro-1-oxo-1,2-dihydroisoquinolin-4-yl)ethyl)-3-fluoro-N-isobutyl-4-(trifluoromethyl)benzamide;
(S)-N-(1-(7,8-difluoro-1-oxo-1,2-dihydroisoquinolin-4-yl)ethyl)-3-fluoro-N-isobutyl-4-(trifluoromethyl)benzamide;
(R)-3-fluoro-N-methyl-N-(1-(1-oxo-1,2-dihydroisoquinolin-4-yl)ethyl)-4-(trifluoromethyl)benzamide;
(S)-3-fluoro-N-methyl-N-(1-(1-oxo-1,2-dihydroisoquinolin-4-yl)ethyl)-4-(trifluoromethyl)benzamide;
(R)-4-bromo-N-methyl-N-(1-(1-oxo-1,2-dihydroisoquinolin-4-yl)ethyl)benzamide;
(S)-4-bromo-N-methyl-N-(1-(1-oxo-1,2-dihydroisoquinolin-4-yl)ethyl)benzamide;
(R)-N-methyl-N-(1-(1-oxo-1,2-dihydroisoquinolin-4-yl)ethyl)-4-(trifluoromethyl)benzamide;
(S)-N-methyl-N-(1-(1-oxo-1,2-dihydroisoquinolin-4-yl)ethyl)-4-(trifluoromethyl)benzamide;
(R)-4-chloro-3-fluoro-N-methyl-N-(1-(1-oxo-1,2-dihydroisoquinolin-4-yl)ethyl)benzamide;
(S)-4-chloro-3-fluoro-N-methyl-N-(1-(1-oxo-1,2-dihydroisoquinolin-4-yl)ethyl)benzamide;
(R)-3-chloro-4-fluoro-N-methyl-N-(1-(1-oxo-1,2-dihydroisoquinolin-4-yl)ethyl)benzamide;
(S)-3-chloro-4-fluoro-N-methyl-N-(1-(1-oxo-1,2-dihydroisoquinolin-4-yl)ethyl)benzamide;
(R)-4-bromo-3-fluoro-N-methyl-N-(1-(1-oxo-1,2-dihydroisoquinolin-4-yl)ethyl)benzamide;
(S)-4-bromo-3-fluoro-N-methyl-N-(1-(1-oxo-1,2-dihydroisoquinolin-4-yl)ethyl)benzamide;
(R)-2-(4-chlorophenyl)-N-methyl-N-(1-(1-oxo-1,2-dihydroisoquinolin-4-yl)ethyl)acetamide;
(S)-2-(4-chlorophenyl)-N-methyl-N-(1-(1-oxo-1,2-dihydroisoquinolin-4-yl)ethyl)acetamide;
(R)-N-methyl-N-(1-(1-oxo-1,2-dihydroisoquinolin-4-yl)ethyl)indolizine-2-carboxamide;
(S)-N-methyl-N-(1-(1-oxo-1,2-dihydroisoquinolin-4-yl)ethyl)indolizine-2-carboxamide;
(R)-7-fluoro-N-methyl-N-(1-(1-oxo-1,2-dihydroisoquinolin-4-yl)ethyl)indolizine-2-carboxamide;
(S)-7-fluoro-N-methyl-N-(1-(1-oxo-1,2-dihydroisoquinolin-4-yl)ethyl)indolizine-2-carboxamide;
(R)-8-chloro-N-methyl-N-(1-(1-oxo-1,2-dihydroisoquinolin-4-yl)ethyl)indolizine-2-carboxamide;
(S)-8-chloro-N-methyl-N-(1-(1-oxo-1,2-dihydroisoquinolin-4-yl)ethyl)indolizine-2-carboxamide;
(R)-N-methyl-N-(1-(1-oxo-1,2-dihydroisoquinolin-4-yl)ethyl)-1H-indole-2-carboxamide;
(S)-N-methyl-N-(1-(1-oxo-1,2-dihydroisoquinolin-4-yl)ethyl)-1H-indole-2-carboxamide;
(R)-4-fluoro-N-methyl-N-(1-(1-oxo-1,2-dihydroisoquinolin-4-yl)ethyl)-1H-indole-2-carboxamide;
(S)-4-fluoro-N-methyl-N-(1-(1-oxo-1,2-dihydroisoquinolin-4-yl)ethyl)-1H-indole-2-carboxamide;
(R)-5-fluoro-N-methyl-N-(1-(1-oxo-1,2-dihydroisoquinolin-4-yl)ethyl)-1H-indole-2-carboxamide;
(S)-5-fluoro-N-methyl-N-(1-(1-oxo-1,2-dihydroisoquinolin-4-yl)ethyl)-1H-indole-2-carboxamide;
(R)-4,6-difluoro-N-methyl-N-(1-(1-oxo-1,2-dihydroisoquinolin-4-yl)ethyl)-1H-indole-2-carboxamide;
(S)-4,6-difluoro-N-methyl-N-(1-(1-oxo-1,2-dihydroisoquinolin-4-yl)ethyl)-1H-indole-2-carboxamide;
(R)-5,6-difluoro-N-methyl-N-(1-(1-oxo-1,2-dihydroisoquinolin-4-yl)ethyl)-1H-indole-2-carboxamide;
(S)-5,6-difluoro-N-methyl-N-(1-(1-oxo-1,2-dihydroisoquinolin-4-yl)ethyl)-1H-indole-2-carboxamide;
(R)-N-methyl-N-((R)-1-(1-oxo-1,2-dihydroisoquinolin-4-yl)ethyl)indoline-2-carboxamide;
(S)-N-methyl-N-((R)-1-(1-oxo-1,2-dihydroisoquinolin-4-yl)ethyl)indoline-2-carboxamide;
(R)-4-bromo-N-(1-(6,7-difluoro-2-methyl-1-oxo-1,2-dihydroisoquinolin-4-yl)ethyl)-3-fluoro-N-methylbenzamide;
(S)-4-bromo-N-(1-(6,7-difluoro-2-methyl-1-oxo-1,2-dihydroisoquinolin-4-yl)ethyl)-3-fluoro-N-methylbenzamide;
(R)-4-bromo-N-(1-(6,7-difluoro-1-methoxyisoquinolin-4-yl)ethyl)-N-methylbenzamide;
(S)-4-bromo-N-(1-(6,7-difluoro-1-methoxyisoquinolin-4-yl)ethyl)-N-methylbenzamide;
(R)-N-(1-(6,7-difluoro-2-methyl-1-oxo-1,2-dihydroisoquinolin-4-yl)ethyl)-3-fluoro-N-methyl-4-(trifluoromethyl)benzamide;
(S)-N-(1-(6,7-difluoro-2-methyl-1-oxo-1,2-dihydroisoquinolin-4-yl)ethyl)-3-fluoro-N-methyl-4-(trifluoromethyl)benzamide;
(R)-4-bromo-N-(1-(6,7-difluoro-2-methyl-1-oxo-1,2-dihydroisoquinolin-4-yl)ethyl)-N-methylbenzamide;
(S)-4-bromo-N-(1-(6,7-difluoro-2-methyl-1-oxo-1,2-dihydroisoquinolin-4-yl)ethyl)-N-methylbenzamide;
(R)-N-(1-(6,7-difluoro-1-methoxyisoquinolin-4-yl)ethyl)-5,6-difluoro-N-methyl-1H-indole-2-carboxamide;
(S)-N-(1-(6,7-difluoro-1-methoxyisoquinolin-4-yl)ethyl)-5,6-difluoro-N-methyl-1H-indole-2-carboxamide;
(R)-N-(1-(6,7-difluoro-2-methyl-1-oxo-1,2-dihydroisoquinolin-4-yl)ethyl)-8-fluoro-N-methylindolizine-2-carboxamide;
(S)-N-(1-(6,7-difluoro-2-methyl-1-oxo-1,2-dihydroisoquinolin-4-yl)ethyl)-8-fluoro-N-methylindolizine-2-carboxamide;
(R)-N-(1-(6,7-difluoro-2-methyl-1-oxo-1,2-dihydroisoquinolin-4-yl)ethyl)-5,6-difluoro-N-methyl-1H-indole-2-carboxamide;
(S)-N-(1-(6,7-difluoro-2-methyl-1-oxo-1,2-dihydroisoquinolin-4-yl)ethyl)-5,6-difluoro-N-methyl-1H-indole-2-carboxamide;
(R)-N-(1-(6,7-difluoro-1-methoxyisoquinolin-4-yl)ethyl)-3-fluoro-N-methyl-4-(trifluoromethyl)benzamide;
(S)-N-(1-(6,7-difluoro-1-methoxyisoquinolin-4-yl)ethyl)-3-fluoro-N-methyl-4-(trifluoromethyl)benzamide;
(R)-4-bromo-N-(1-(6,7-difluoro-1-methoxyisoquinolin-4-yl)ethyl)-3-fluoro-N-methylbenzamide;
(S)-4-bromo-N-(1-(6,7-difluoro-1-methoxyisoquinolin-4-yl)ethyl)-3-fluoro-N-methylbenzamide;
(R)-N-(1-(6,7-difluoro-1-methoxyisoquinolin-4-yl)ethyl)-8-fluoro-N-methylindolizine-2-carboxamide;
(S)-N-(1-(6,7-difluoro-1-methoxyisoquinolin-4-yl)ethyl)-8-fluoro-N-methylindolizine-2-carboxamide;
(2S)-N-(1-(6,7-difluoro-1-methoxyisoquinolin-4-yl)ethyl)-N-methylindoline-2-carboxamide;
(S)-N-((S)-1-(6,7-difluoro-1-methoxyisoquinolin-4-yl)ethyl)-N-methylindoline-2-carboxamide;
(S)-N-((R)-1-(6,7-difluoro-1-methoxyisoquinolin-4-yl)ethyl)-N-methylindoline-2-carboxamide;
(2R)-N-(1-(6,7-difluoro-1-methoxyisoquinolin-4-yl)ethyl)-N-methylindoline-2-carboxamide;
(R)-N-((S)-1-(6,7-difluoro-1-methoxyisoquinolin-4-yl)ethyl)-N-methylindoline-2-carboxamide;
(R)-N-((R)-1-(6,7-difluoro-1-methoxyisoquinolin-4-yl)ethyl)-N-methylindoline-2-carboxamide;
(2S)-N-(1-(6,7-difluoro-2-methyl-1-oxo-1,2-dihydroisoquinolin-4-yl)ethyl)-N-methylindoline-2-carboxamide;
(S)-N-((S)-1-(6,7-difluoro-2-methyl-1-oxo-1,2-dihydroisoquinolin-4-yl)ethyl)-N-methylindoline-2-carboxamide;
(S)-N-((R)-1-(6,7-difluoro-2-methyl-1-oxo-1,2-dihydroisoquinolin-4-yl)ethyl)-N-methylindoline-2-carboxamide;
(2R)-N-(1-(6,7-difluoro-2-methyl-1-oxo-1,2-dihydroisoquinolin-4-yl)ethyl)-N-methylindoline-2-carboxamide;
(R)-N-((S)-1-(6,7-difluoro-2-methyl-1-oxo-1,2-dihydroisoquinolin-4-yl)ethyl)-N-methylindoline-2-carboxamide;
(R)-N-((R)-1-(6,7-difluoro-2-methyl-1-oxo-1,2-dihydroisoquinolin-4-yl)ethyl)-N-methylindoline-2-carboxamide;
(R)-N-(1-(6,7-difluoro-1-oxo-1,2-dihydroisoquinolin-4-yl)ethyl)-N-methylindolizine-6-carboxamide;
(S)-N-(1-(6,7-difluoro-1-oxo-1,2-dihydroisoquinolin-4-yl)ethyl)-N-methylindolizine-6-carboxamide;
(R)-2-chloro-N-(1-(6,7-difluoro-1-oxo-1,2-dihydroisoquinolin-4-yl)ethyl)-N-methyl-4H-thieno[3,2-b]pyrrole-5-carboxamide;
(S)-2-chloro-N-(1-(6,7-difluoro-1-oxo-1,2-dihydroisoquinolin-4-yl)ethyl)-N-methyl-4H-thieno[3,2-b]pyrrole-5-carboxamide;
(R)-N-(1-(6,7-difluoro-1-oxo-1,2-dihydroisoquinolin-4-yl)ethyl)-N-methylindolizine-7-carboxamide;
(S)-N-(1-(6,7-difluoro-1-oxo-1,2-dihydroisoquinolin-4-yl)ethyl)-N-methylindolizine-7-carboxamide;
   or a salt, solvate, prodrug, isotopically labeled derivative, stereoisomer, or tautomer thereof, or any mixtures thereof.

Embodiment 17 provides a pharmaceutical composition comprising at least one compound of any of Embodiments 1-16 and a pharmaceutically acceptable carrier.

Embodiment 18 provides the pharmaceutical composition of Embodiment 17, further comprising at least one additional agent useful for treating, ameliorating, and/or preventing hepatitis infection.

Embodiment 19 provides the pharmaceutical composition of Embodiment 18, wherein the at least one additional agent comprises at least one selected from the group consisting of reverse transcriptase inhibitor; capsid inhibitor; cccDNA formation inhibitor; RNA destabilizer; oligomeric nucleotide targeted against the HBV genome; immunostimulator; GalNAc-siRNA conjugate targeted against an HBV gene transcript; and therapeutic vaccine.

Embodiment 20 provides the pharmaceutical composition of Embodiment 19, wherein the immunostimulator is a checkpoint inhibitor.

Embodiment 21 provides the pharmaceutical composition of Embodiment 20, wherein the checkpoint inhibitor is a PD-L1 inhibitor.

Embodiment 22 provides a method of treating, ameliorating, and/or preventing hepatitis B virus (HBV) infection in a subject, the method comprising administering to the subject in need thereof a therapeutically effective amount of at least one compound of any of Embodiments 1-16 and/or at least one pharmaceutical composition of any Embodiments 17-21.

Embodiment 23 provides the method of Embodiment 22, wherein the subject is further infected with hepatitis D virus (HDV).

Embodiment 24 provides the method of any of Embodiments 22-23, wherein the at least one compound and/or composition is administered to the subject in a pharmaceutically acceptable composition.

Embodiment 25 provides the method of any of Embodiments 22-24, wherein the subject is further administered at least one additional agent useful for treating, ameliorating, and/or preventing the hepatitis B virus infection.

Embodiment 26 provides the method of Embodiment 25, wherein the at least one additional agent comprises at least one selected from the group consisting of reverse transcriptase inhibitor; capsid inhibitor; cccDNA formation inhibitor; RNA destabilizer; oligomeric nucleotide targeted against the HBV genome; immunostimulator; GalNAc-siRNA conjugate targeted against an HBV gene transcript; and therapeutic vaccine.

Embodiment 27 provides the method of Embodiment 26, wherein the immunostimulator is a checkpoint inhibitor.

Embodiment 28 provides the method of Embodiment 27, wherein the checkpoint inhibitor is a PD-L1 inhibitor.

Embodiment 29 provides the method of any of Embodiments 25-28, wherein the subject is co-administered the at least one compound and/or composition and the at least one additional agent.

Embodiment 30 provides the method of any of Embodiments 25-29, wherein the at least one compound and/or composition and the at least one additional agent are coformulated.

Embodiment 31 provides a method of inhibiting expression and/or function of a viral capsid protein directly or indirectly in a heptatis B virus-infected subject, the method comprising administering to the subject in need thereof a therapeutically effective amount of at least one compound of any of Embodiments 1-16 and/or at least one pharmaceutical composition of any of Embodiments 17-21.

Embodiment 32 provides the method of Embodiment 31, wherein the subject is further infected with hepatitis D virus (HDV).

Embodiment 33 provides the method of any of Embodiments 31-32, wherein the at least one compound and/or composition is administered to the subject in a pharmaceutically acceptable composition.

Embodiment 34 provides the method of any of Embodiments 31-33, wherein the subject is further administered at least one additional agent useful for treating, ameliorating, and/or preventing the hepatitis B viral infection.

Embodiment 35 provides the method of Embodiment 34, wherein the at least one additional agent comprises at least one selected from the group consisting of reverse transcriptase inhibitor; capsid inhibitor; cccDNA formation inhibitor; RNA destabilizer; oligomeric nucleotide targeted against the HBV genome; immunostimulator; GalNAc-siRNA conjugate targeted against an HBV gene transcript; and therapeutic vaccine.

Embodiment 36 provides the method of Embodiment 35, wherein the immunostimulator is a checkpoint inhibitor.

Embodiment 37 provides the method of Embodiment 36, wherein the checkpoint inhibitor is a PD-L1 inhibitor.

Embodiment 38 provides the method of any of Embodiments 34-37, wherein the subject is co-administered the at least one compound and/or composition and the at least one additional agent.

Embodiment 39 provides the method of any of Embodiments 34-38, wherein the at least one compound and/or composition and the at least one additional agent are coformulated.

Embodiment 40 provides the method of any of Embodiments 22-39, wherein the subject is a mammal.

Embodiment 41 provides the method of Embodiment 40, wherein the mammal is a human.

The disclosures of each and every patent, patent application, and publication cited herein are hereby incorporated herein by reference in their entirety. While this disclosure has been disclosed with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

1. A compound of formula (I), or a salt, solvate, prodrug, stereoisomer, tautomer, or isotopically labeled derivative thereof, or any mixtures thereof: 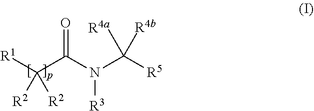
wherein:
R¹ is selected from the group consisting of 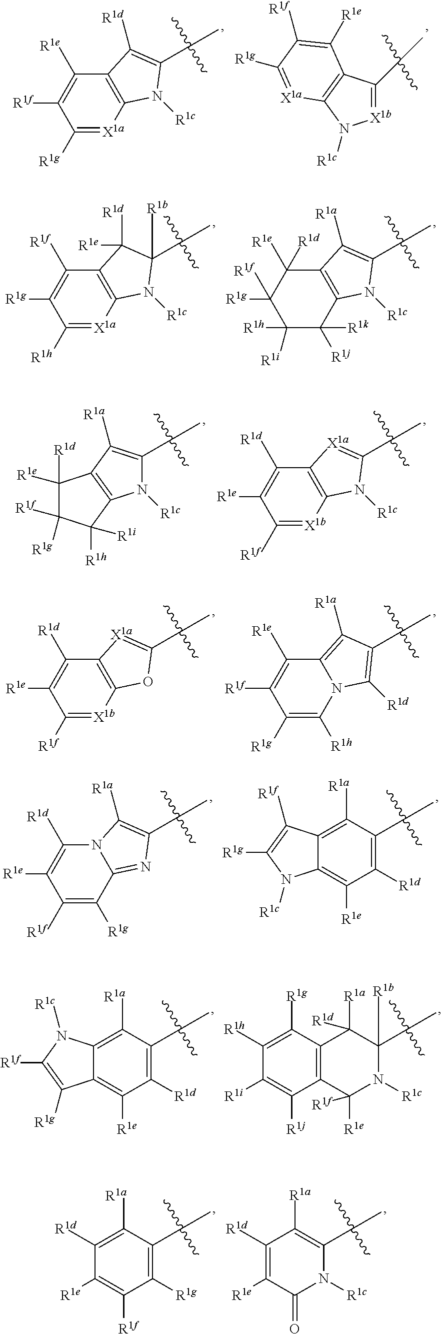 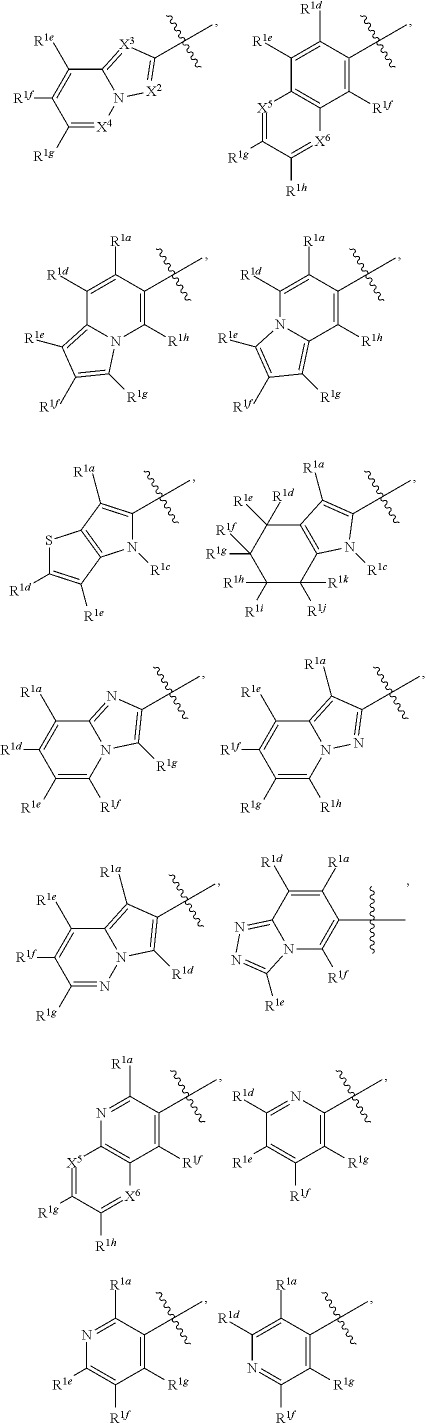 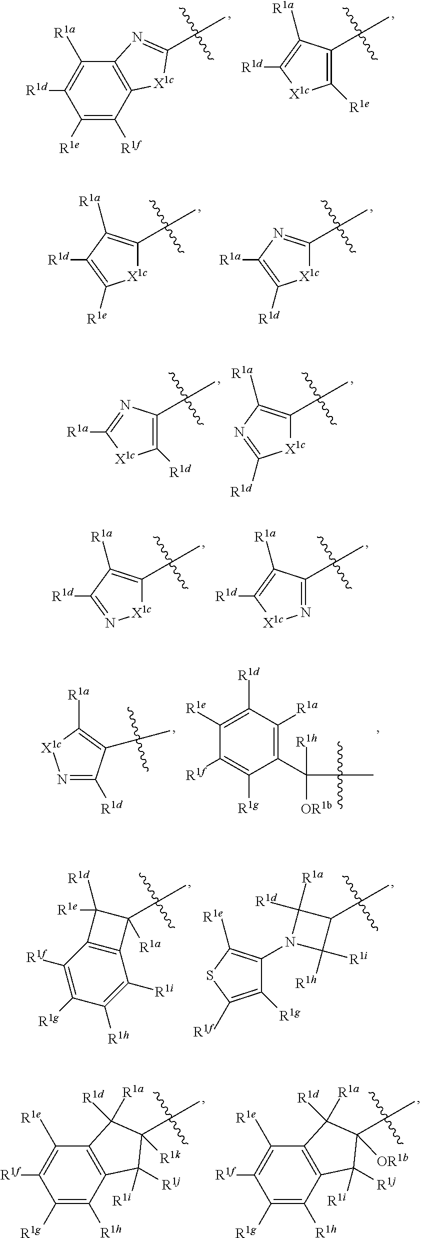 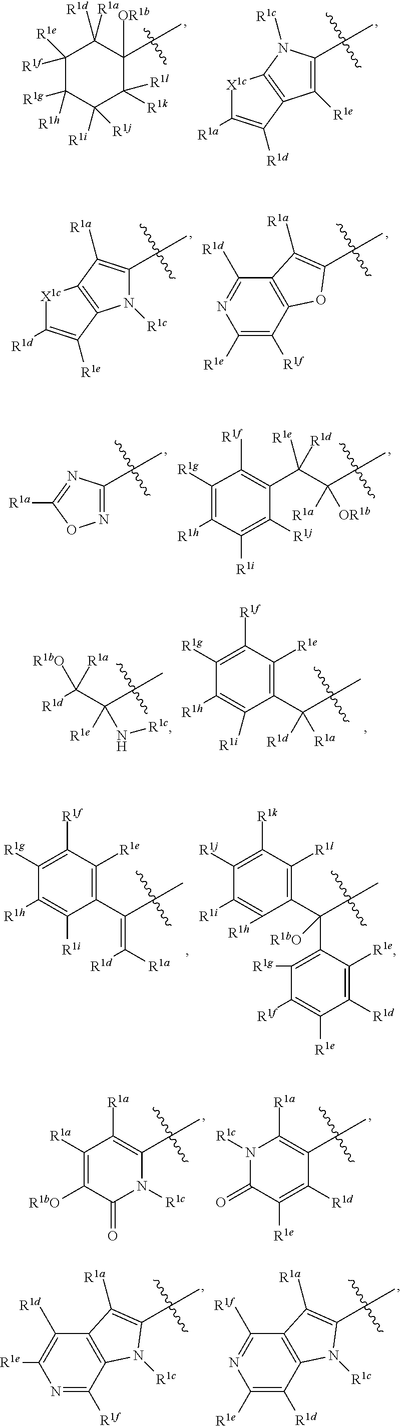 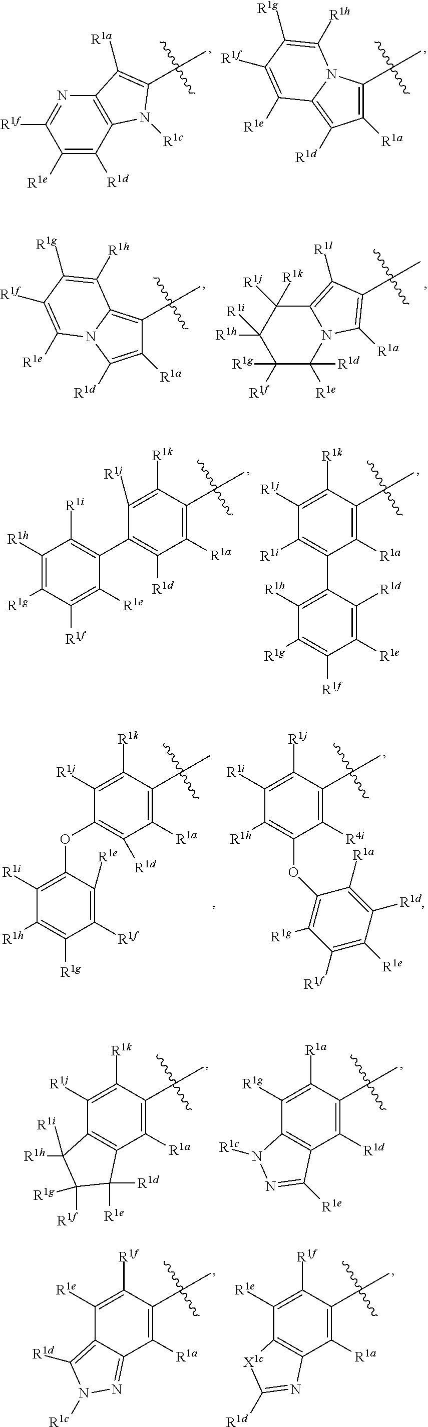 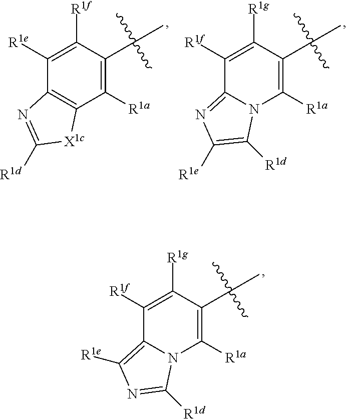
optionally substituted C₃-C₈ cycloalkyl, —NH(optionally substituted C₃-C₈ cycloalkyl), and —NH(optionally substituted phenyl);
each occurrence of R^{1a}, R^{1d}, R^{1e}, R^{1f}, R^{1g}, R^{1h}, R¹ⁱ, R^{1j}, R^{1k} and R^{1l} is independently selected from the group consisting of H, halogen, —CN, optionally substituted C₁-C₃ alkyl, optionally substituted C₃-C₈ cycloalkyl, optionally substituted C₁-C₆ alkoxy, optionally substituted C₃-C₈ cycloalkoxy, heterocyclyl, heteroaryl, —S(optionally substituted C₁-C₆ alkyl), —SO(optionally substituted C₁-C₆ alkyl), —SO₂(optionally substituted C₁-C₆ alkoxy), —C(═O)OH, —C(═O)O(optionally substituted C₁-C₆ alkyl), —C(═O)O(optionally substituted C₃-C₈ cycloalkyl), —O(optionally substituted C₁-C₆ alkyl), —O(optionally substituted C₃-C₈ cycloalkyl), —NH₂, —NH(optionally substituted C₁-C₆ alkyl), —NH(optionally substituted C₃-C₈ cycloalkyl), —N(optionally substituted C₁-C₆ alkyl)(optionally substituted C₁-C₆ alkyl), —N(optionally substituted C₃-C₈ cycloalkyl)(optionally substituted C₃-C₈ cycloalkyl), —N(optionally substituted C₁-C₆ alkyl)(optionally substituted C₃-C₈ cycloalkyl), —C(═O)NH₂, —C(═O)NH(optionally substituted C₁-C₆ alkyl), —C(═O)NH(optionally substituted C₃-C₈ cycloalkyl), —C(═O)N(optionally substituted C₁-C₆ alkyl)(optionally substituted C₁-C₆ alkyl), —C(═O)N(optionally substituted C₃-C₈ cycloalkyl)(optionally substituted C₃-C₈ cycloalkyl), and —C(═O)N(optionally substituted C₁-C₆ alkyl)(optionally substituted C₃-C₈ cycloalkyl;
each occurrence of R^{1b} and R^{1c} is independently H, optionally substituted C₁-C₆ alkyl, or optionally substituted C₃-C₈ cycloalkyl;
each occurrence of X^{1a} is independently N or C(R^{1a});
each occurrence of X^{1b} is independently N or C(R^{1d});
each occurrence of X^{1c} is independently N(R^{1b}), O, or S;
one of the following applies:
(i) X² is N, X³ is C(R^{1a}), and X⁴ is C(R^{1d});
(ii) X³ is N, X² is C(R^{1a}), and X⁴ is C(R^{1d}); or
(iii) X⁴ is N, X² is C(R^{1a}), and X³ is C(R^{1d});
one of the following applies:
(i) X⁵ is N and X⁶ is C(R^{1a}); or
(ii) X⁶ is N and X⁵ is C(R^{1a});
each occurrence of R² is independently H, optionally substituted C₁-C₆ alkyl, or optionally substituted C₃-C₈ cycloalkyl, or the two R² groups combine to form ═O;
R³ is selected from the group consisting of H, C₁-C₆ alkyl, and C₃-C₈ cycloalkyl, wherein the alkyl or cycloalkyl is optionally substituted with at least one substituent selected from the group consisting of C₁-C₆ alkyl, C₃-C₈ cycloalkyl, halogen, cyano, —OH, C₁-C₆ alkoxy, C₃-C₈ cycloalkoxy, C₁-C₆ haloalkoxy, C₃-C₈ halocycloalkoxy, optionally substituted phenyl, optionally substituted heteroaryl, optionally substituted heterocyclyl, —C(═O)OR⁶, —OC(═O)R⁶, —SR⁶, —S(═O)R⁶, —S(═O)₂R⁶, —S(═O)₂NR⁶R⁶, —N(R⁶)S(═O)₂R⁶, —N(R⁶)C(═O)R⁶, —C(═O)NR⁶R⁶, and —NR⁶R⁶;
R^{4a} is selected from the group consisting of H, C₁-C₆ alkyl, C₃-C₈ cycloalkyl, and phenyl, wherein the alkyl, cycloalkyl, or phenyl is optionally substituted with at least one substituent selected from the group consisting of C₁-C₆ alkyl, C₃-C₈ cycloalkyl, halogen, cyano, —OH, C₁-C₆ alkoxy, C₃-C₈ cycloalkoxy, C₁-C₆ haloalkoxy, C₃-C₈ halocycloalkoxy, —NR⁶R⁶, and optionally substituted phenyl;
R^{4b} is selected from the group consisting of H and optionally substituted C₁-C₆ alkyl;
R⁵ is selected from the group consisting of: 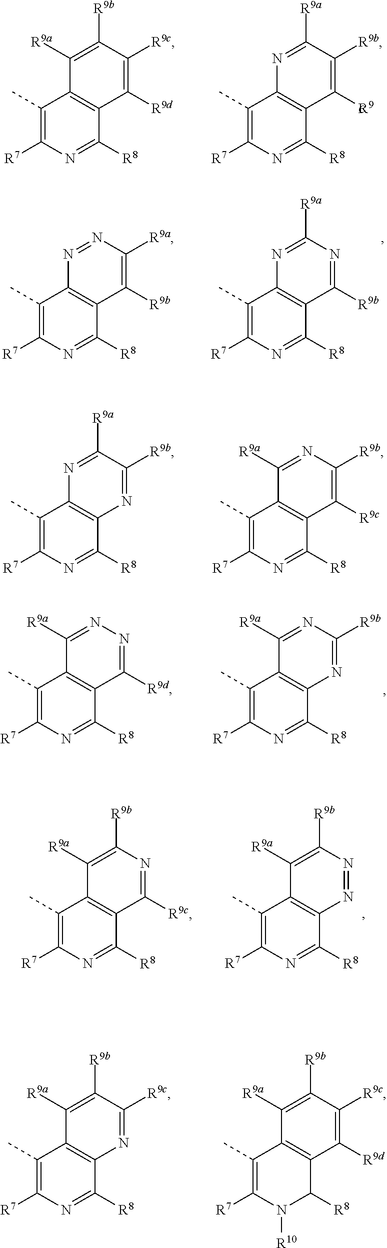 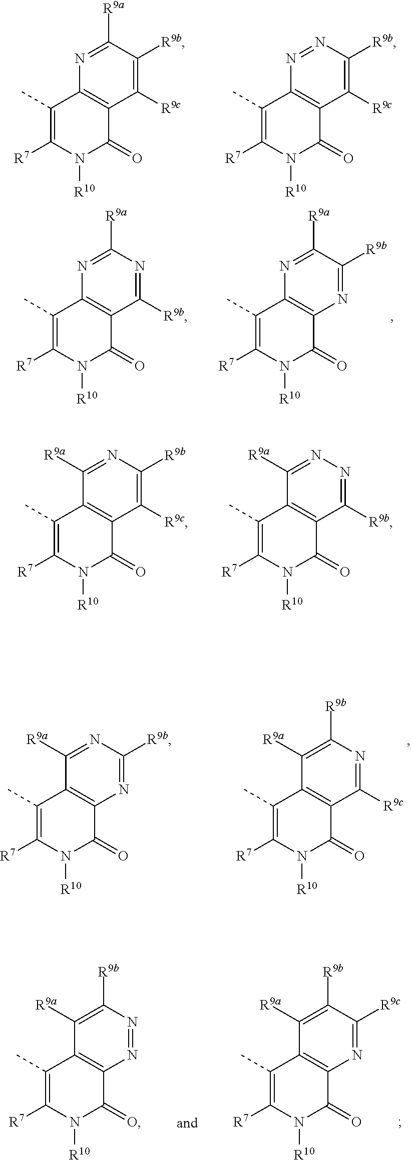
each occurrence of R⁶ is independently selected from the group consisting of H, optionally substituted C₁-C₆ alkyl, optionally substituted C₃-C₈ cycloalkyl, optionally substituted phenyl, and optionally substituted hetereoaryl;
each occurrence of R⁷ is independently selected from the group consisting of H, halogen, optionally substituted C₁-C₆ alkyl, optionally substituted C₃-C₈ cycloalkyl, optionally substituted C₁-C₆ alkoxy, and optionally substituted C₃-C₈ cycloalkoxy;
each occurrence of R⁸ is independently selected from the group consisting of H, halogen, —CN, optionally substituted C₁-C₆ alkyl, optionally substituted C₃-C₈ cycloalkyl, optionally substituted C₁-C₆ alkoxy, optionally substituted C₃-C₈ cycloalkoxy, heterocyclyl, heteroaryl, —S(optionally substituted C₁-C₆ alkyl), —SO(optionally substituted C₁-C₆ alkyl), —SO₂(optionally substituted C₁-C₆ alkyl), —C(═O)OH, —C(═O)O(optionally substituted C₁-C₆ alkyl), —C(═O)O(optionally substituted C₃-C₈ cycloalkyl), —O(optionally substituted C₁-C₆ alkyl), —O(optionally substituted C₃-C₈ cycloalkyl), —NH₂, —NH(optionally substituted C₁-C₆ alkyl), —NH(optionally substituted C₃-C₈ cycloalkyl), —N(optionally substituted C₁-C₆ alkyl)(optionally substituted C₁-C₆ alkyl), —N(optionally substituted C₃-C₈ cycloalkyl)(optionally substituted C₃-C₈ cycloalkyl), —N(optionally substituted C₁-C₆ alkyl)(optionally substituted C₃-C₈ cycloalkyl), —C(═O)NH₂, —C(═O)NH(optionally substituted C₁-C₆ alkyl), —C(═O)NH(optionally substituted C₃-C₈ cycloalkyl), —C(═O)N(optionally substituted C₁-C₆ alkyl)(optionally substituted C₁-C₆ alkyl), —C(═O)N(optionally substituted C₃-C₈ cycloalkyl)(optionally substituted C₃-C₈ cycloalkyl), and —C(═O)N(optionally substituted C₁-C₆ alkyl)(optionally substituted C₃-C₈ cycloalkyl;
each occurrence of R^{9a}, R^{9b}, R^{9c}, and R^{9d} is independently selected from the group consisting of H, halogen, optionally substituted C₁-C₆ alkyl, optionally substituted C₃-C₈ cycloalkyl, optionally substituted C₁-C₆ alkoxy, and optionally substituted C₃-C₈ cycloalkoxy;
each occurrence of R¹⁰ is independently selected from the group consisting of H, optionally substituted C₁-C₆ alkyl, and optionally substituted C₃-C₈ cycloalkyl; and
each occurrence of p is 0 or 1,
wherein if p is 0, then R¹ is not —NH(optionally substituted C₃-C₈ cycloalkyl) or —NH(optionally substituted phenyl).

2. The compound of claim 1, wherein each occurrence of aryl or heteroaryl is independently optionally substituted with at least one substituent selected from the group consisting of C₁-C₆ alkyl, C₃-C₈ cycloalkyl, phenyl, C₁-C₆ hydroxyalkyl, (C₁-C₆ alkoxy)-C₁-C₆ alkyl, C₁-C₆ haloalkyl, C₁-C₆ haloalkoxy, halogen, —CN, —OR^{b}, —N(R^{b})(R^{b}), —NO₂, —C(═O)N(R^{b})(R^{b}), —C(═O)OR^{b}, —OC(═O)R^{b}, —SR^{b}, —S(═O)R^{b}, —S(═O)₂R^{b}, N(R^{b})S(═O)₂R^{b}, —S(═O)₂N(R^{b})(R^{b}), acyl, and C₁-C₆ alkoxycarbonyl, wherein each occurrence of R^{b} is independently H, C₁-C₆ alkyl, or C₃-C₈ cycloalkyl, wherein in R^{b} the alkyl or cycloalkyl is optionally substituted with at least one selected from the group consisting of halogen, —OH, C₁-C₆ alkoxy, and heteroaryl; or substituents on two adjacent carbon atoms combine to form —O(CH₂)₁₋₃O—.

3. The compound of claim 1, wherein each occurrence of alkyl, alkenyl, alkynyl, or cycloalkyl is independently optionally substituted with at least one substituent selected from the group consisting of C₁-C₆ alkyl, C₃-C₈ cycloalkyl, halo, cyano (—CN), —OR^{a}, optionally substituted phenyl, optionally substituted heteroaryl, optionally substituted heterocyclyl, —C(═O)OR^{a}, —OC(═O)R^{a}, —SR^{a}, —S(═O)R^{a}, —S(═O)₂R^{a}, —S(═O)₂NR^{a}R^{a}, —N(R^{a})S(═O)₂R^{a}, —N(R^{a})C(═O)R^{a}, —C(═O)NR^{a}R^{a}, and —N(R^{a})(R^{a}), wherein each occurrence of R^{a} is independently H, optionally substituted C₁-C₆ alkyl, optionally substituted C₃-C₈ cycloalkyl, optionally substituted aryl, or optionally substituted heteroaryl, or two R^{a} groups combine with the N to which they are bound to form a heterocycle.

4. The compound of claim 1, which is at least one selected from the group consisting of:

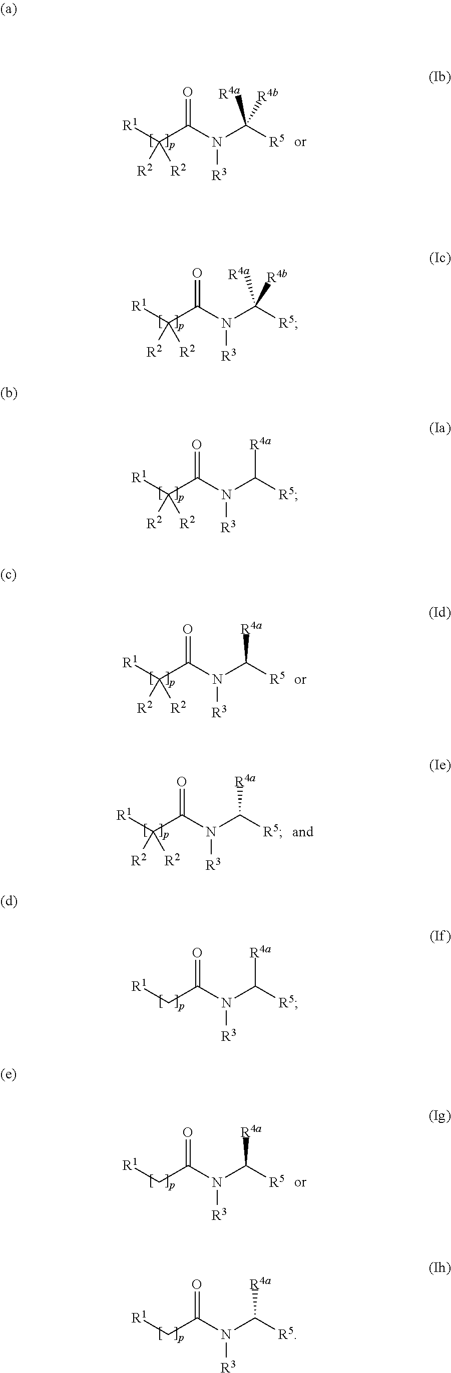

5-8. (canceled)

9. The compound of claim 1, wherein R¹ is selected from the group consisting of:

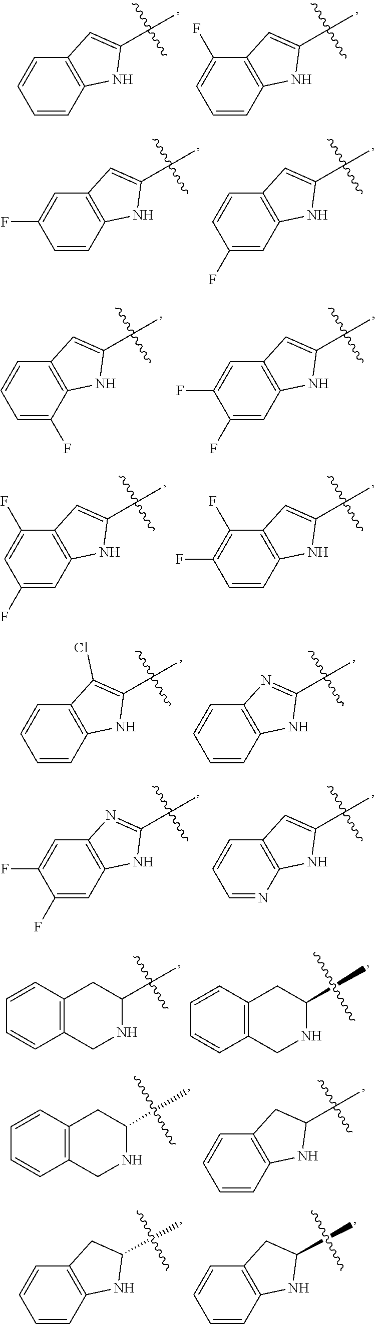

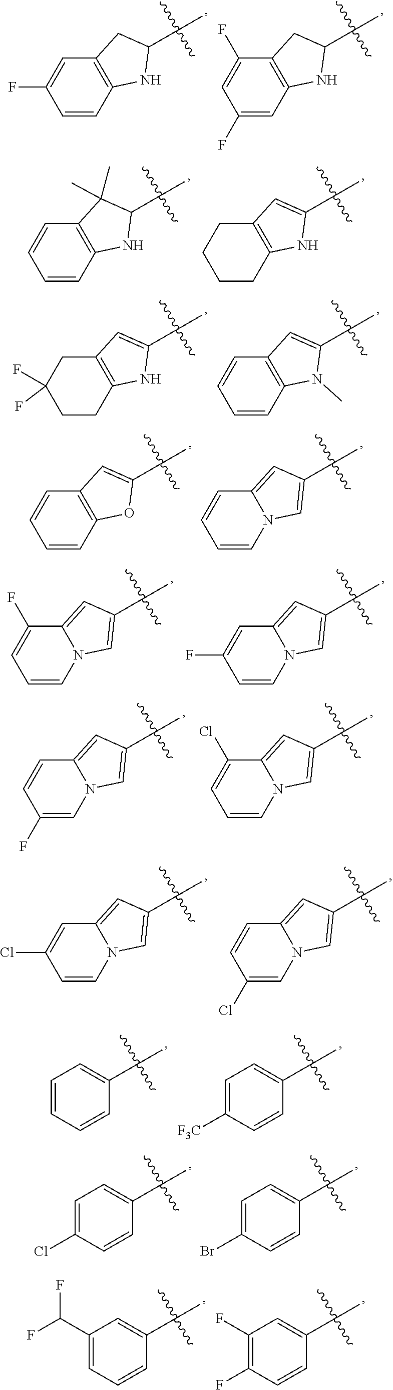

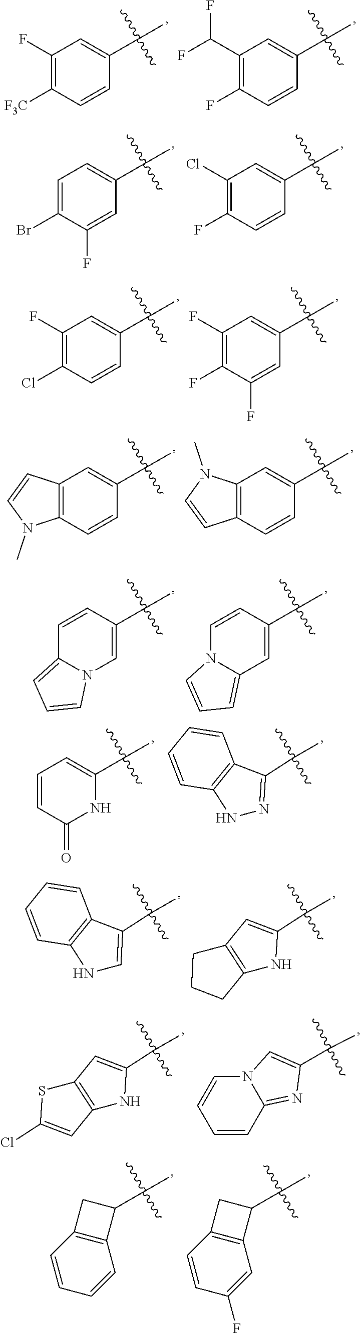

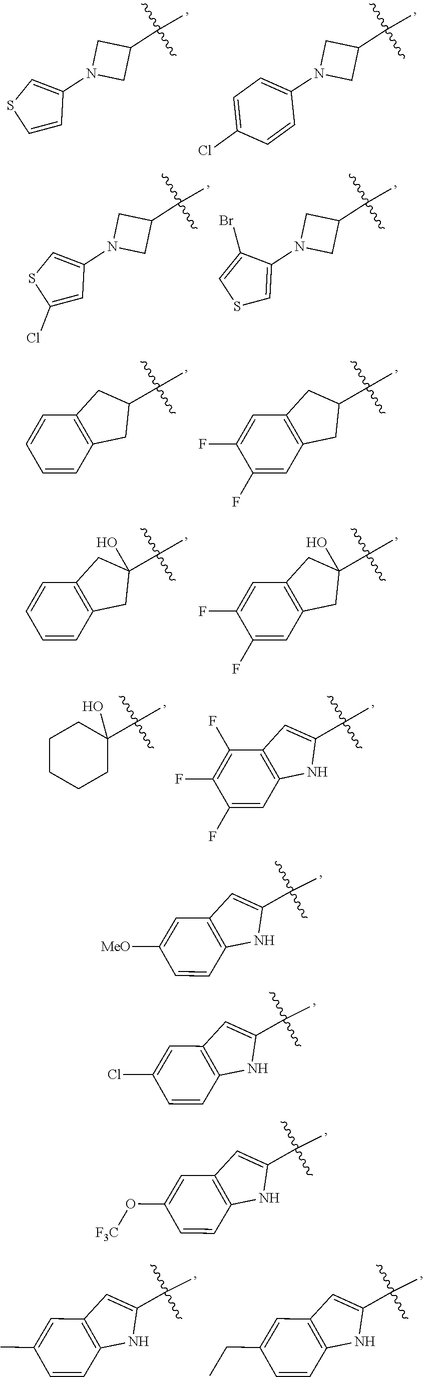

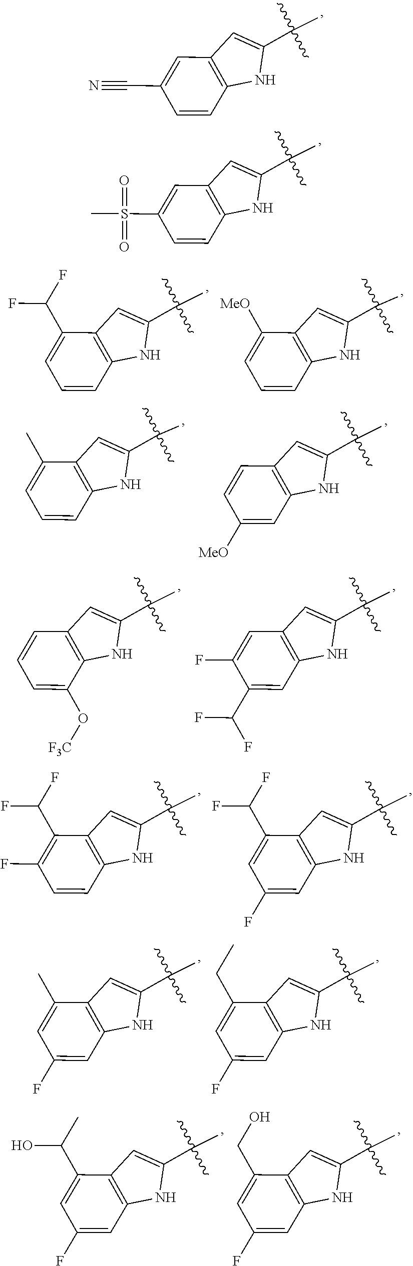

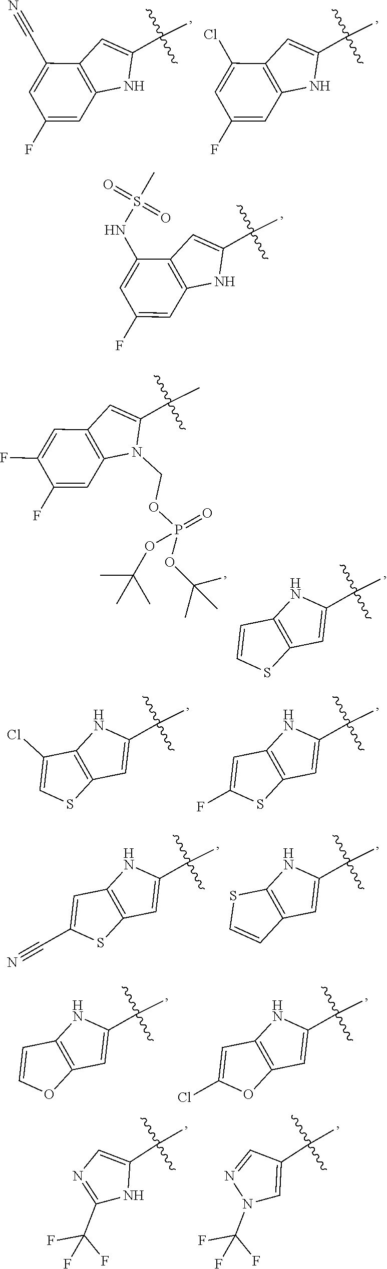

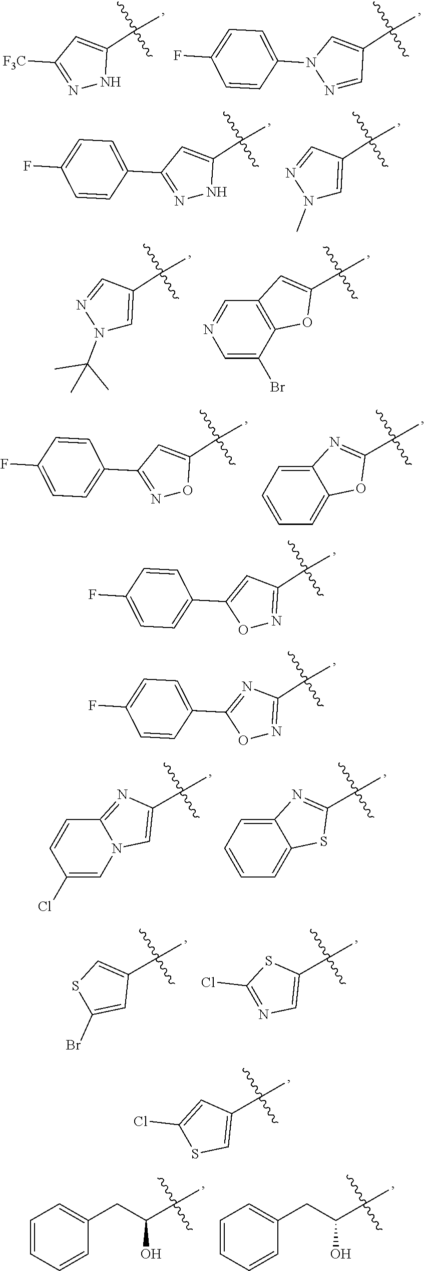

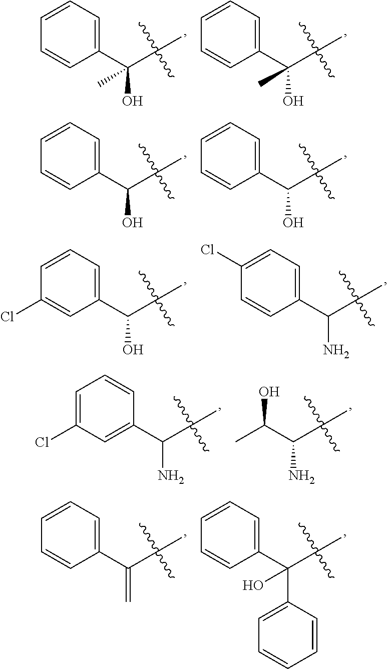

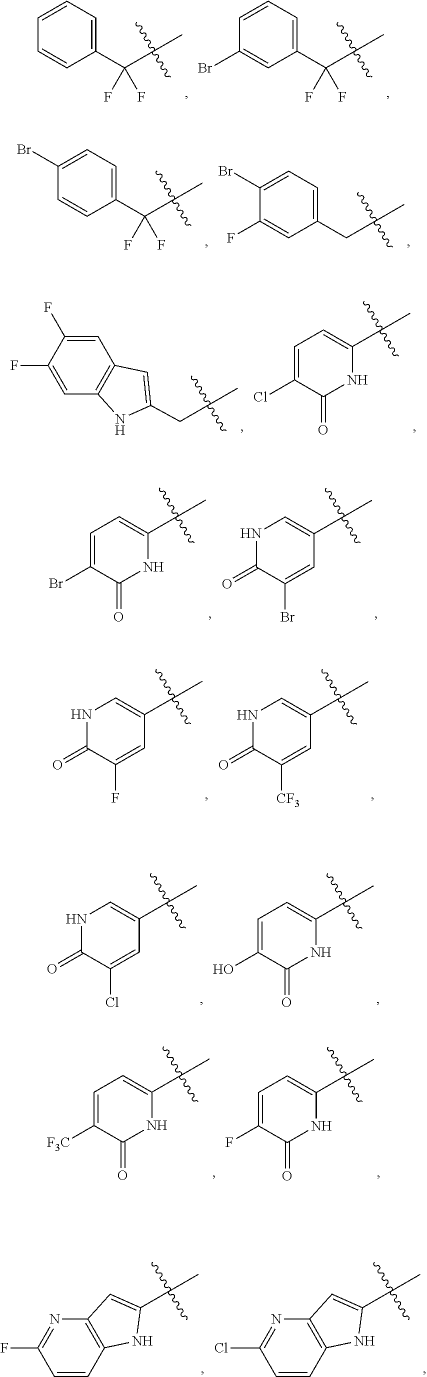

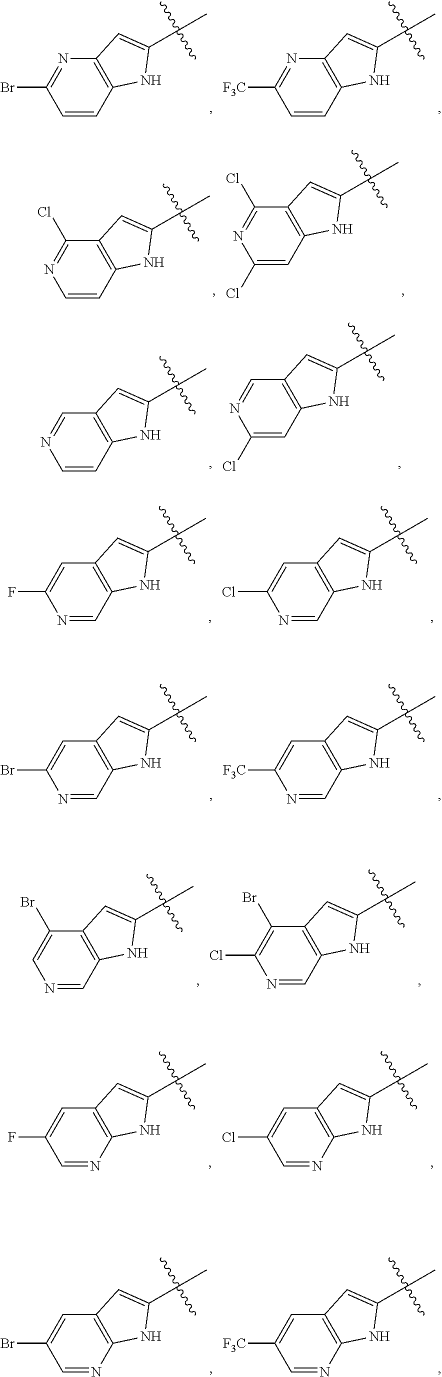

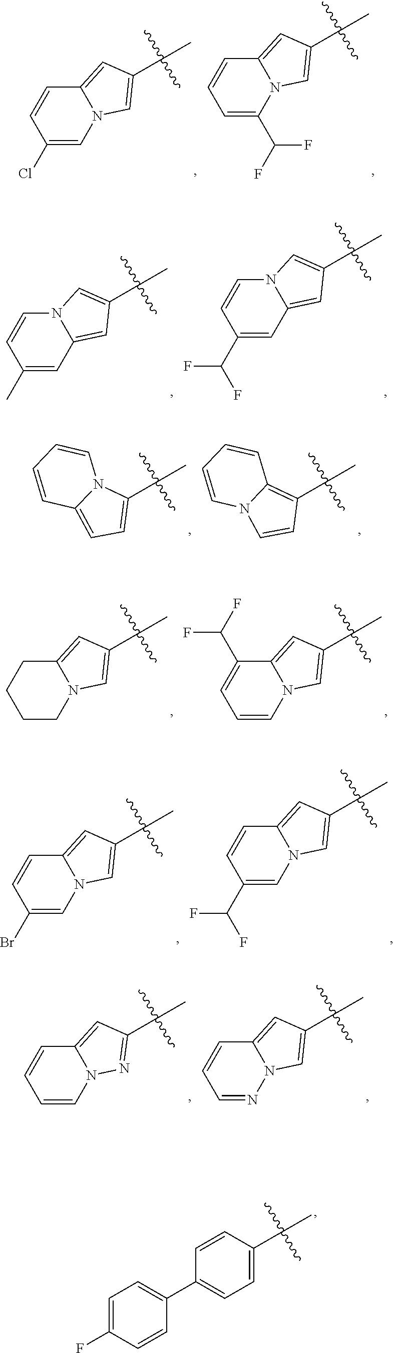

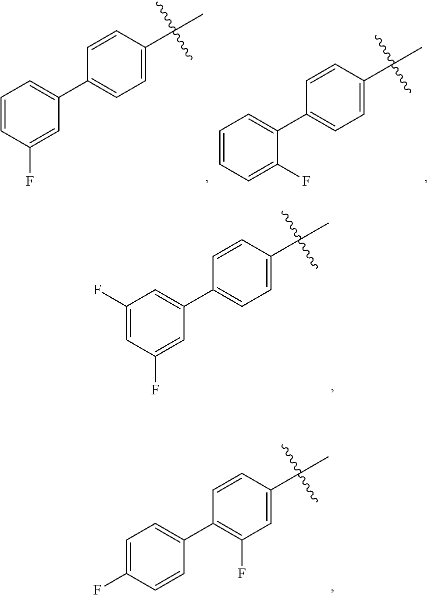 [SMILES: *c1ccc(-c2cccc(F)c2)cc1 ; *c1ccc(-c2ccccc2F)cc1 ; *c1ccc(-c2cc(F)cc(F)c2)cc1 ; *c1ccc(-c2ccc(F)cc2)c(F)c1]

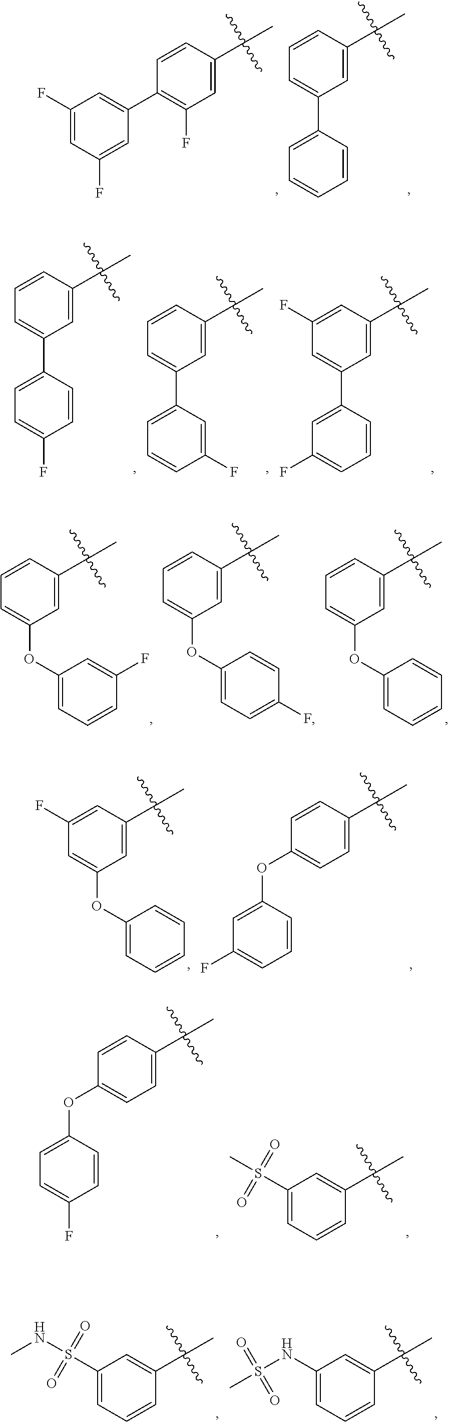

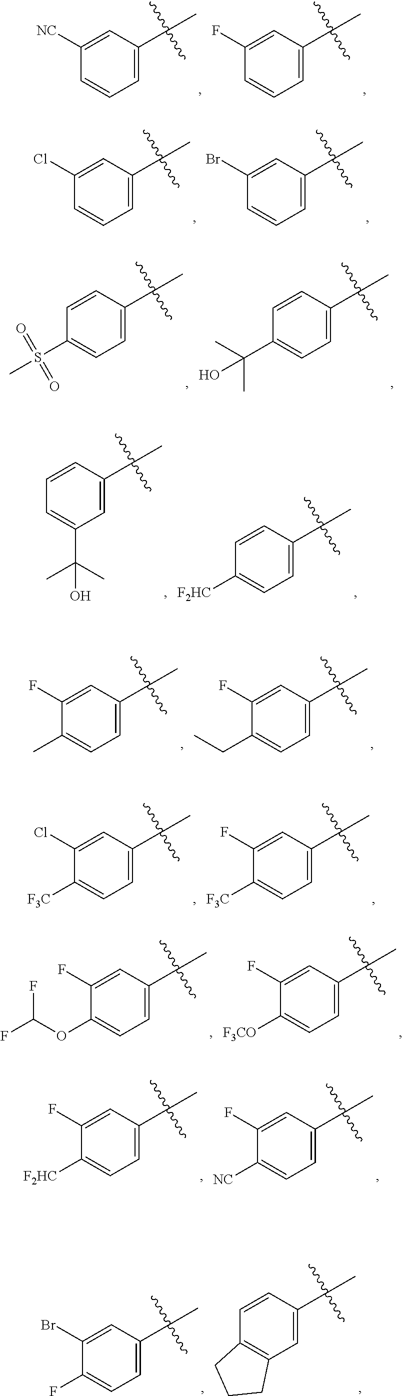

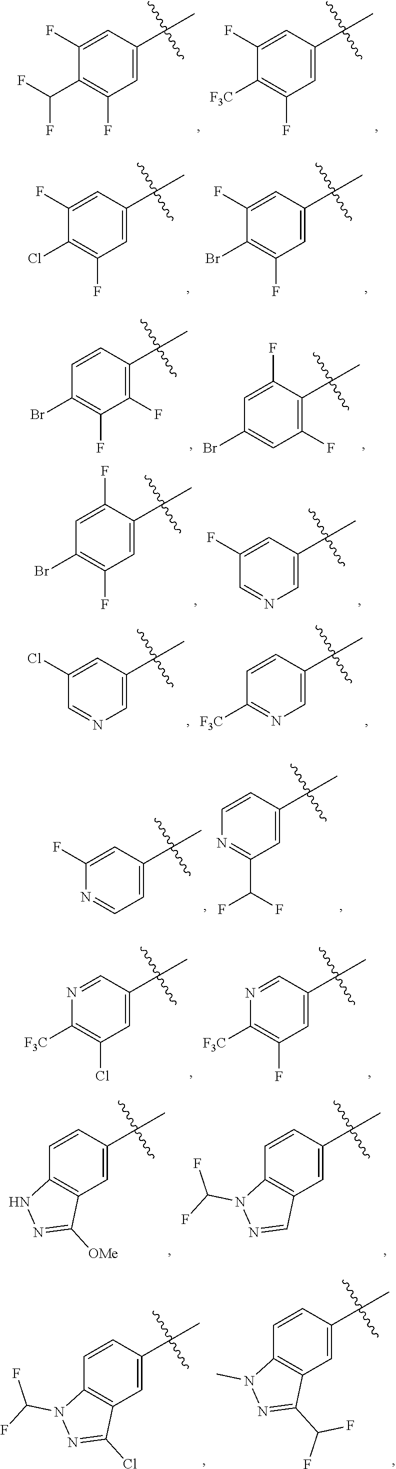

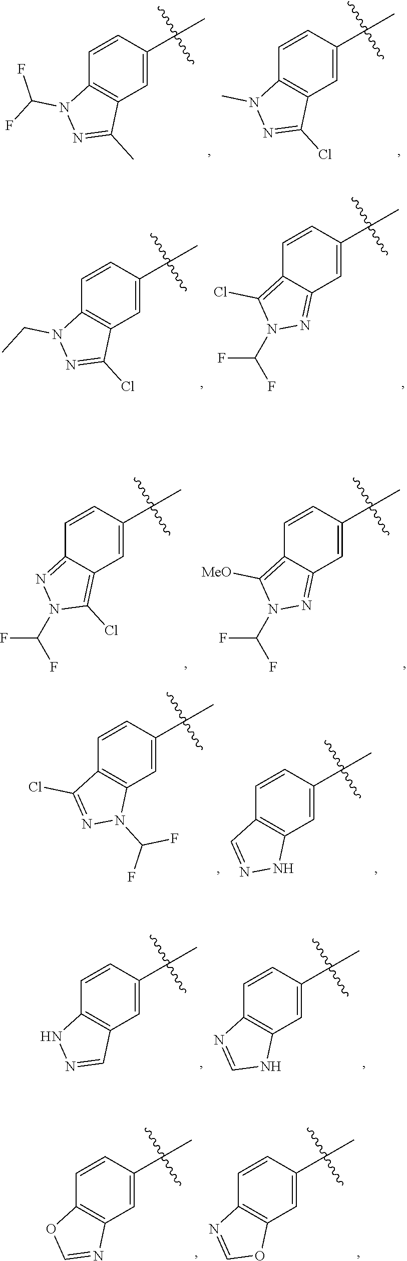

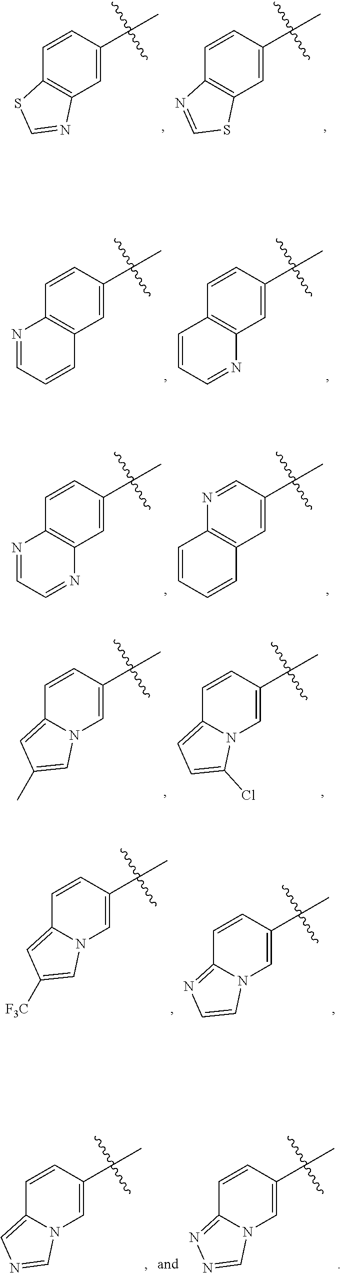

10. The compound of claim 1, wherein each occurrence of R² is independently H or methyl.

11. The compound of claim 1, wherein R³ is selected from the group consisting of H, methyl, ethyl, isopropyl, n-propyl, cyclopropyl, n-butyl, isobutyl, sec-butyl, tert-butyl, cyclobutyl, isopropylmethyl, —(CH₂)₂₋₆OH, —(CH₂)₂₋₆O(C₁-C₆ alkyl), optionally substituted benzyl, and optionally substituted phenyl.

12. The compound of claim 1, wherein each of R^{4a} and R^{4b} is independently selected from the group consisting of H and methyl.

13. The compound of claim 1, wherein R⁵ is selected from the group consisting of:

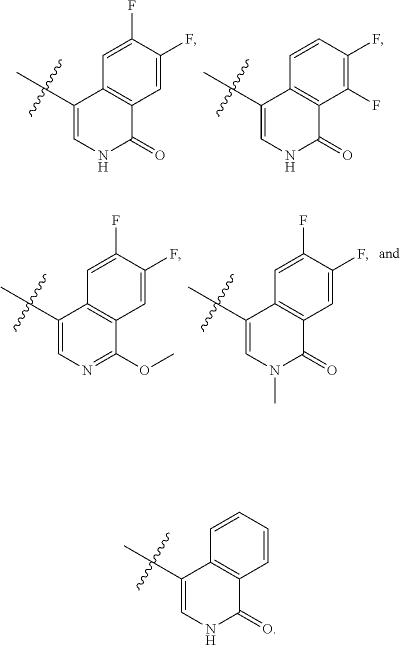 [SMILES: *c1c[nH]c(=O)c2cc(F)c(F)cc12 ; *c1c[nH]c(=O)c2c(F)c(F)ccc12 ; *c1cnc(OC)c2cc(F)c(F)cc12 ; *c1cn(C)c(=O)c2cc(F)c(F)cc12 ; *c1c[nH]c(=O)c2ccccc12]

14. The compound of claim 1, wherein p is 0.

15. The compound of claim 1, which is at least one selected from the group consisting of:
N-(1-(6,7-difluoro-1-oxo-1,2-dihydroisoquinolin-4-yl)ethyl)-N-methyl-1H-indole-2-carboxamide;
N-(1-(6,7-difluoro-1-oxo-1,2-dihydroisoquinolin-4-yl)ethyl)-N-isobutyl-1H-indole-2-carboxamide;
N-(1-(6,7-difluoro-1-oxo-1,2-dihydroisoquinolin-4-yl)ethyl)-N-ethyl-1H-indole-2-carboxamide;
N-(1-(6,7-difluoro-1-oxo-1,2-dihydroisoquinolin-4-yl)ethyl)-4-fluoro-N-methyl-1H-indole-2-carboxamide;
N-(1-(6,7-difluoro-1-oxo-1,2-dihydroisoquinolin-4-yl)ethyl)-5-fluoro-N-methyl-1H-indole-2-carboxamide;
N-(1-(6,7-difluoro-1-oxo-1,2-dihydroisoquinolin-4-yl)ethyl)-6-fluoro-N-methyl-1H-indole-2-carboxamide;
N-(1-(6,7-difluoro-1-oxo-1,2-dihydroisoquinolin-4-yl)ethyl)-7-fluoro-N-methyl-1H-indole-2-carboxamide;
N-(1-(6,7-difluoro-1-oxo-1,2-dihydroisoquinolin-4-yl)ethyl)-5,6-difluoro-N-methyl-1H-indole-2-carboxamide;
N-(1-(6,7-difluoro-1-oxo-1,2-dihydroisoquinolin-4-yl)ethyl)-4,6-difluoro-N-methyl-1H-indole-2-carboxamide;
N-(1-(6,7-difluoro-1-oxo-1,2-dihydroisoquinolin-4-yl)ethyl)-N-methyl-1,2,3,4-tetrahydroisoquinoline-3-carboxamide;
N-(1-(6,7-difluoro-1-oxo-1,2-dihydroisoquinolin-4-yl)ethyl)-N-methylindoline-2-carboxamide;
N-(1-(6,7-difluoro-1-oxo-1,2-dihydroisoquinolin-4-yl)ethyl)-N-methyl-1H-benzo[d]imidazole-2-carboxamide;
N-(1-(6,7-difluoro-1-oxo-1,2-dihydroisoquinolin-4-yl)ethyl)-N-methylbenzofuran-2-carboxamide;
N-(1-(6,7-difluoro-1-oxo-1,2-dihydroisoquinolin-4-yl)ethyl)-N-(3-hydroxypropyl)-1H-indole-2-carboxamide;
N-(1-(6,7-difluoro-1-oxo-1,2-dihydroisoquinolin-4-yl)ethyl)-4,5-difluoro-N-methyl-1H-indole-2-carboxamide;
N-(1-(6,7-difluoro-1-oxo-1,2-dihydroisoquinolin-4-yl)ethyl)-N-methylindoline-2-carboxamide;
N-(1-(6,7-difluoro-1-oxo-1,2-dihydroisoquinolin-4-yl)ethyl)-N-methyl-1,2,3,4-tetrahydroisoquinoline-3-carboxamide;
N-(1-(6,7-difluoro-1-oxo-1,2-dihydroisoquinolin-4-yl)ethyl)-N-methyl-4,5,6,7-tetrahydro-1H-indole-2-carboxamide;
N-(1-(6,7-difluoro-1-oxo-1,2-dihydroisoquinolin-4-yl)ethyl)-N,1-dimethyl-1H-indole-2-carboxamide;
N-(1-(6,7-difluoro-1-oxo-1,2-dihydroisoquinolin-4-yl)ethyl)-N-methylimidazo[1,2-a]pyridine-2-carboxamide;
N-(1-(6,7-difluoro-1-oxo-1,2-dihydroisoquinolin-4-yl)ethyl)-N-methyl-1,4,5,6-tetrahydrocyclopenta[b]pyrrole-2-carboxamide;
N-(1-(6,7-difluoro-1-oxo-1,2-dihydroisoquinolin-4-yl)ethyl)-5,6-difluoro-N-methyl-1H-benzo[d]imidazole-2-carboxamide;
N-(1-(6,7-difluoro-1-oxo-1,2-dihydroisoquinolin-4-yl)ethyl)-N-methyl-1H-pyrrolo[2,3-b]pyridine-2-carboxamide;
N-(1-(6,7-difluoro-1-oxo-1,2-dihydroisoquinolin-4-yl)ethyl)-N-methyl-1H-indole-3-carboxamide;
N-(1-(6,7-difluoro-1-oxo-1,2-dihydroisoquinolin-4-yl)ethyl)-N-methyl-1H-indazole-3-carboxamide;
N-(1-(7,8-difluoro-1-oxo-1,2-dihydroisoquinolin-4-yl)ethyl)-N-methyl-1,4,5,6-tetrahydrocyclopenta[b]pyrrole-2-carboxamide;
N-(1-(6,7-difluoro-1-oxo-1,2-dihydroisoquinolin-4-yl)ethyl)-2-(1H-indol-2-yl)-N-methylacetamide;
3-chloro-N-(1-(6,7-difluoro-1-oxo-1,2-dihydroisoquinolin-4-yl)ethyl)-N-methyl-1H-indole-2-carboxamide;
N-(1-(6,7-difluoro-1-oxo-1,2-dihydroisoquinolin-4-yl)ethyl)-5-fluoro-N-methylindoline-2-carboxamide;
N-(1-(7,8-difluoro-1-oxo-1,2-dihydroisoquinolin-4-yl)ethyl)-4,6-difluoro-N-methylindoline-2-carboxamide;
N-(1-(6,7-difluoro-1-oxo-1,2-dihydroisoquinolin-4-yl)ethyl)-N-methylindolizine-2-carboxamide;
N-(1-(7,8-difluoro-1-oxo-1,2-dihydroisoquinolin-4-yl)ethyl)-N-methylindolizine-2-carboxamide;
N-(1-(7,8-difluoro-1-oxo-1,2-dihydroisoquinolin-4-yl)ethyl)-5-fluoro-N-methylindoline-2-carboxamide;
8-chloro-N-(1-(6,7-difluoro-1-oxo-1,2-dihydroisoquinolin-4-yl)ethyl)-N-methylindolizine-2-carboxamide;
N-(1-(6,7-difluoro-1-oxo-1,2-dihydroisoquinolin-4-yl)ethyl)-8-fluoro-N-methylindolizine-2-carboxamide;
N-(1-(7,8-difluoro-1-oxo-1,2-dihydroisoquinolin-4-yl)ethyl)-8-fluoro-N-methylindolizine-2-carboxamide;
N-(1-(6,7-difluoro-1-oxo-1,2-dihydroisoquinolin-4-yl)ethyl)-N-(3-hydroxypropyl)-2-(1H-indol-2-yl)acetamide;
N-(1-(7,8-difluoro-1-oxo-1,2-dihydroisoquinolin-4-yl)ethyl)-N-methyl-6-oxo-1,6-dihydropyridine-2-carboxamide;
N-(1-(6,7-difluoro-1-oxo-1,2-dihydroisoquinolin-4-yl)ethyl)-5,5-difluoro-N-methyl-4,5,6,7-tetrahydro-1H-indole-2-carboxamide;
N-(1-(7,8-difluoro-1-oxo-1,2-dihydroisoquinolin-4-yl)ethyl)-5,5-difluoro-N-methyl-4,5,6,7-tetrahydro-1H-indole-2-carboxamide;
8-chloro-N-(1-(7,8-difluoro-1-oxo-1,2-dihydroisoquinolin-4-yl)ethyl)-N-methylindolizine-2-carboxamide;
3-chloro-N-(1-(6,7-difluoro-1-oxo-1,2-dihydroisoquinolin-4-yl)ethyl)-N-(3-hydroxypropyl)-1H-indole-2-carboxamide;
7-chloro-N-(1-(6,7-difluoro-1-oxo-1,2-dihydroisoquinolin-4-yl)ethyl)-N-methylindolizine-2-carboxamide;
N-(1-(6,7-difluoro-1-oxo-1,2-dihydroisoquinolin-4-yl)ethyl)-4,6-difluoro-N-methylindoline-2-carboxamide;
N-(1-(6,7-difluoro-1-oxo-1,2-dihydroisoquinolin-4-yl)ethyl)-6-fluoro-N-methylindolizine-2-carboxamide;
N-(1-(7,8-difluoro-1-oxo-1,2-dihydroisoquinolin-4-yl)ethyl)-N,3,3-trimethylindoline-2-carboxamide;
N-(1-(6,7-difluoro-1-oxo-1,2-dihydroisoquinolin-4-yl)ethyl)-7-fluoro-N-methylindolizine-2-carboxamide;
6-chloro-N-(1-(6,7-difluoro-1-oxo-1,2-dihydroisoquinolin-4-yl)ethyl)-N-methylindolizine-2-carboxamide;
N-(1-(6,7-difluoro-1-oxo-1,2-dihydroisoquinolin-4-yl)ethyl)-N,3,3-trimethylindoline-2-carboxamide;
N-(1-(6,7-Difluoro-1-oxo-1,2-dihydroisoquinolin-4-yl)ethyl)-N-isobutylindolizine-2-carboxamide;
N-(1-(6,7-difluoro-1-oxo-1,2-dihydroisoquinolin-4-yl)ethyl)-3-fluoro-N-isobutyl-4-(trifluoromethyl)benzamide;
N-(1-(6,7-Difluoro-1-oxo-1,2-dihydroisoquinolin-4-yl)ethyl)-3-fluoro-N-methyl-4-(trifluoromethyl) benzamide;
4-Chloro-N-(1-(6,7-difluoro-1-oxo-1,2-dihydroisoquinolin-4-yl)ethyl)-3-fluoro-N-methylbenzamide;
N-(1-(6,7-Difluoro-1-oxo-1,2-dihydroisoquinolin-4-yl)ethyl)-3,4,5-trifluoro-N-methylbenzamide;
N-(1-(6,7-Difluoro-1-oxo-1,2-dihydroisoquinolin-4-yl)ethyl)-3-(difluoromethyl)-N-methylbenzamide;
N-(1-(6,7-Difluoro-1-oxo-1,2-dihydroisoquinolin-4-yl)ethyl)-N-methylbenzamide
N-(1-(6,7-Difluoro-1-oxo-1,2-dihydroisoquinolin-4-yl)ethyl)-N-methyl-4-(trifluoromethyl) benzamide;
4-Chloro-N-(1-(6,7-difluoro-1-oxo-1,2-dihydroisoquinolin-4-yl)ethyl)-N-methylbenzamide;
N-(1-(6,7-Difluoro-1-oxo-1,2-dihydroisoquinolin-4-yl)ethyl)-3-(difluoromethyl)-4-fluoro-N-methylbenzamide;
3-Chloro-N-(1-(6,7-difluoro-1-oxo-1,2-dihydroisoquinolin-4-yl)ethyl)-4-fluoro-N-methylbenzamide;
N-(1-(6,7-Difluoro-1-oxo-1,2-dihydroisoquinolin-4-yl)ethyl)-3,4-difluoro-N-methylbenzamide;
N-(1-(6,7-Difluoro-1-oxo-1,2-dihydroisoquinolin-4-yl)ethyl)-N,1-dimethyl-1H-indole-5-carboxamide;
N-(1-(6,7-Difluoro-1-oxo-1,2-dihydroisoquinolin-4-yl)ethyl)-N,1-dimethyl-1H-indole-6-carboxamide;
N1-Cyclopropyl-N2-(1-(6,7-difluoro-1-oxo-1,2-dihydroisoquinolin-4-yl)ethyl)-N2-methyloxalamide;
N1-(3-Chloro-4-fluorophenyl)-N2-(1-(6,7-difluoro-1-oxo-1,2-dihydroisoquinolin-4-yl)ethyl)-N2-methyloxalamide;
N-(1-(7,8-difluoro-1-oxo-1,2-dihydroisoquinolin-4-yl)ethyl)-3-fluoro-N-isobutyl-4-(trifluoromethyl)benzamide;
3-fluoro-N-methyl-N-(1-(1-oxo-1,2-dihydroisoquinolin-4-yl)ethyl)-4-(trifluoromethyl)benzamide;
4-bromo-N-methyl-N-(1-(1-oxo-1,2-dihydroisoquinolin-4-yl)ethyl)benzamide;
N-methyl-N-(1-(1-oxo-1,2-dihydroisoquinolin-4-yl)ethyl)-4-(trifluoromethyl)benzamide;
4-chloro-3-fluoro-N-methyl-N-(1-(1-oxo-1,2-dihydroisoquinolin-4-yl)ethyl)benzamide;
3-chloro-4-fluoro-N-methyl-N-(1-(1-oxo-1,2-dihydroisoquinolin-4-yl)ethyl)benzamide;
4-bromo-3-fluoro-N-methyl-N-(1-(1-oxo-1,2-dihydroisoquinolin-4-yl)ethyl)benzamide;
2-(4-chlorophenyl)-N-methyl-N-(1-(1-oxo-1,2-dihydroisoquinolin-4-yl)ethyl)acetamide;
N-methyl-N-(1-(1-oxo-1,2-dihydroisoquinolin-4-yl)ethyl)indolizine-2-carboxamide;
7-fluoro-N-methyl-N-(1-(1-oxo-1,2-dihydroisoquinolin-4-yl)ethyl)indolizine-2-carboxamide;
8-chloro-N-methyl-N-(1-(1-oxo-1,2-dihydroisoquinolin-4-yl)ethyl)indolizine-2-carboxamide;
N-methyl-N-(1-(1-oxo-1,2-dihydroisoquinolin-4-yl)ethyl)-1H-indole-2-carboxamide;
4-fluoro-N-methyl-N-(1-(1-oxo-1,2-dihydroisoquinolin-4-yl)ethyl)-1H-indole-2-carboxamide;
5-fluoro-N-methyl-N-(1-(1-oxo-1,2-dihydroisoquinolin-4-yl)ethyl)-1H-indole-2-carboxamide;
4,6-difluoro-N-methyl-N-(1-(1-oxo-1,2-dihydroisoquinolin-4-yl)ethyl)-1H-indole-2-carboxamide;
5,6-difluoro-N-methyl-N-(1-(1-oxo-1,2-dihydroisoquinolin-4-yl)ethyl)-1H-indole-2-carboxamide;
N-methyl-N-((R)-1-(1-oxo-1,2-dihydroisoquinolin-4-yl)ethyl)indoline-2-carboxamide;
4-bromo-N-(1-(6,7-difluoro-2-methyl-1-oxo-1,2-dihydroisoquinolin-4-yl)ethyl)-3-fluoro-N-methylbenzamide;
4-bromo-N-(1-(6,7-difluoro-1-methoxyisoquinolin-4-yl)ethyl)-N-methylbenzamide;
N-(1-(6,7-difluoro-2-methyl-1-oxo-1,2-dihydroisoquinolin-4-yl)ethyl)-3-fluoro-N-methyl-4-(trifluoromethyl)benzamide;
4-bromo-N-(1-(6,7-difluoro-2-methyl-1-oxo-1,2-dihydroisoquinolin-4-yl)ethyl)-N-methylbenzamide;
N-(1-(6,7-difluoro-1-methoxyisoquinolin-4-yl)ethyl)-5,6-difluoro-N-methyl-1H-indole-2-carboxamide;
N-(1-(6,7-difluoro-2-methyl-1-oxo-1,2-dihydroisoquinolin-4-yl)ethyl)-8-fluoro-N-methylindolizine-2-carboxamide;
N-(1-(6,7-difluoro-2-methyl-1-oxo-1,2-dihydroisoquinolin-4-yl)ethyl)-5,6-difluoro-N-methyl-1H-indole-2-carboxamide;
N-(1-(6,7-difluoro-1-methoxyisoquinolin-4-yl)ethyl)-3-fluoro-N-methyl-4-(trifluoromethyl)benzamide;
4-bromo-N-(1-(6,7-difluoro-1-methoxyisoquinolin-4-yl)ethyl)-3-fluoro-N-methylbenzamide;
N-(1-(6,7-difluoro-1-methoxyisoquinolin-4-yl)ethyl)-8-fluoro-N-methylindolizine-2-carboxamide;
N-(1-(6,7-difluoro-1-methoxyisoquinolin-4-yl)ethyl)-N-methylindoline-2-carboxamide;
N-(1-(6,7-difluoro-2-methyl-1-oxo-1,2-dihydroisoquinolin-4-yl)ethyl)-N-methylindoline-2-carboxamide;
N-(1-(6,7-difluoro-1-oxo-1,2-dihydroisoquinolin-4-yl)ethyl)-N-methylindolizine-6-carboxamide;
2-chloro-N-(1-(6,7-difluoro-1-oxo-1,2-dihydroisoquinolin-4-yl)ethyl)-N-methyl-4H-thieno[3,2-b]pyrrole-5-carboxamide; and
N-(1-(6,7-difluoro-1-oxo-1,2-dihydroisoquinolin-4-yl)ethyl)-N-methylindolizine-7-carboxamide;
or a salt, solvate, prodrug, isotopically labeled derivative, stereoisomer, or tautomer thereof, or any mixtures thereof.

16. The compound of claim 1, which is at least one selected from the group consisting of:
(R)-N-(1-(6,7-difluoro-1-oxo-1,2-dihydroisoquinolin-4-yl)ethyl)-N-methyl-1H-indole-2-carboxamide;
(S)-N-(1-(6,7-difluoro-1-oxo-1,2-dihydroisoquinolin-4-yl)ethyl)-N-methyl-1H-indole-2-carboxamide;
(R)-N-(1-(6,7-difluoro-1-oxo-1,2-dihydroisoquinolin-4-yl)ethyl)-N-isobutyl-1H-indole-2-carboxamide;
(S)-N-(1-(6,7-difluoro-1-oxo-1,2-dihydroisoquinolin-4-yl)ethyl)-N-isobutyl-1H-indole-2-carboxamide;
(R)-N-(1-(6,7-difluoro-1-oxo-1,2-dihydroisoquinolin-4-yl)ethyl)-N-ethyl-1H-indole-2-carboxamide;
(S)-N-(1-(6,7-difluoro-1-oxo-1,2-dihydroisoquinolin-4-yl)ethyl)-N-ethyl-1H-indole-2-carboxamide;
(R)-N-(1-(6,7-difluoro-1-oxo-1,2-dihydroisoquinolin-4-yl)ethyl)-4-fluoro-N-methyl-1H-indole-2-carboxamide;
(S)-N-(1-(6,7-difluoro-1-oxo-1,2-dihydroisoquinolin-4-yl)ethyl)-4-fluoro-N-methyl-1H-indole-2-carboxamide;
(R)-N-(1-(6,7-difluoro-1-oxo-1,2-dihydroisoquinolin-4-yl)ethyl)-5-fluoro-N-methyl-1H-indole-2-carboxamide;
(S)-N-(1-(6,7-difluoro-1-oxo-1,2-dihydroisoquinolin-4-yl)ethyl)-5-fluoro-N-methyl-1H-indole-2-carboxamide;
(R)-N-(1-(6,7-difluoro-1-oxo-1,2-dihydroisoquinolin-4-yl)ethyl)-6-fluoro-N-methyl-1H-indole-2-carboxamide;
(S)-N-(1-(6,7-difluoro-1-oxo-1,2-dihydroisoquinolin-4-yl)ethyl)-6-fluoro-N-methyl-1H-indole-2-carboxamide;
(R)-N-(1-(6,7-difluoro-1-oxo-1,2-dihydroisoquinolin-4-yl)ethyl)-7-fluoro-N-methyl-1H-indole-2-carboxamide;
(S)-N-(1-(6,7-difluoro-1-oxo-1,2-dihydroisoquinolin-4-yl)ethyl)-7-fluoro-N-methyl-1H-indole-2-carboxamide;
(R)-N-(1-(6,7-difluoro-1-oxo-1,2-dihydroisoquinolin-4-yl)ethyl)-5,6-difluoro-N-methyl-1H-indole-2-carboxamide;
(S)-N-(1-(6,7-difluoro-1-oxo-1,2-dihydroisoquinolin-4-yl)ethyl)-5,6-difluoro-N-methyl-1H-indole-2-carboxamide;
(R)-N-(1-(6,7-difluoro-1-oxo-1,2-dihydroisoquinolin-4-yl)ethyl)-4,6-difluoro-N-methyl-1H-indole-2-carboxamide;
(S)-N-(1-(6,7-difluoro-1-oxo-1,2-dihydroisoquinolin-4-yl)ethyl)-4,6-difluoro-N-methyl-1H-indole-2-carboxamide;
(3R)-N-((1R)-(6,7-difluoro-1-oxo-1,2-dihydroisoquinolin-4-yl)ethyl)-N-methyl-1,2,3,4-tetrahydroisoquinoline-3-carboxamide;
(3R)-N-((1S)-(6,7-difluoro-1-oxo-1,2-dihydroisoquinolin-4-yl)ethyl)-N-methyl-1,2,3,4-tetrahydroisoquinoline-3-carboxamide;
(3S)-N-((1R)-(6,7-difluoro-1-oxo-1,2-dihydroisoquinolin-4-yl)ethyl)-N-methyl-1,2,3,4-tetrahydroisoquinoline-3-carboxamide;
(3S)-N-((1S)-(6,7-difluoro-1-oxo-1,2-dihydroisoquinolin-4-yl)ethyl)-N-methyl-1,2,3,4-tetrahydroisoquinoline-3-carboxamide;
(2R)-N-((1R)-(6,7-difluoro-1-oxo-1,2-dihydroisoquinolin-4-yl)ethyl)-N-methylindoline-2-carboxamide;
(2R)-N-((1S)-(6,7-difluoro-1-oxo-1,2-dihydroisoquinolin-4-yl)ethyl)-N-methylindoline-2-carboxamide;
(2S)-N-((1R)-(6,7-difluoro-1-oxo-1,2-dihydroisoquinolin-4-yl)ethyl)-N-methylindoline-2-carboxamide;
(2S)-N-((1S)-(6,7-difluoro-1-oxo-1,2-dihydroisoquinolin-4-yl)ethyl)-N-methylindoline-2-carboxamide;
(R)-N-(1-(6,7-difluoro-1-oxo-1,2-dihydroisoquinolin-4-yl)ethyl)-N-methyl-1H-benzo[d]imidazole-2-carboxamide;
(S)-N-(1-(6,7-difluoro-1-oxo-1,2-dihydroisoquinolin-4-yl)ethyl)-N-methyl-1H-benzo[d]imidazole-2-carboxamide;
(R)-N-(1-(6,7-difluoro-1-oxo-1,2-dihydroisoquinolin-4-yl)ethyl)-N-methylbenzofuran-2-carboxamide;
(S)-N-(1-(6,7-difluoro-1-oxo-1,2-dihydroisoquinolin-4-yl)ethyl)-N-methylbenzofuran-2-carboxamide;
(R)-N-(1-(6,7-difluoro-1-oxo-1,2-dihydroisoquinolin-4-yl)ethyl)-N-(3-hydroxypropyl)-1H-indole-2-carboxamide;
(S)-N-(1-(6,7-difluoro-1-oxo-1,2-dihydroisoquinolin-4-yl)ethyl)-N-(3-hydroxypropyl)-1H-indole-2-carboxamide;
(R)-N-(1-(6,7-difluoro-1-oxo-1,2-dihydroisoquinolin-4-yl)ethyl)-4,5-difluoro-N-methyl-1H-indole-2-carboxamide;
(S)-N-(1-(6,7-difluoro-1-oxo-1,2-dihydroisoquinolin-4-yl)ethyl)-4,5-difluoro-N-methyl-1H-indole-2-carboxamide;
(2R)-N-((1R)-(6,7-difluoro-1-oxo-1,2-dihydroisoquinolin-4-yl)ethyl)-N-methylindoline-2-carboxamide;
(2R)-N-((1S)-(6,7-difluoro-1-oxo-1,2-dihydroisoquinolin-4-yl)ethyl)-N-methylindoline-2-carboxamide;
(2S)-N-((1R)-(6,7-difluoro-1-oxo-1,2-dihydroisoquinolin-4-yl)ethyl)-N-methylindoline-2-carboxamide;
(2S)-N-((1S)-(6,7-difluoro-1-oxo-1,2-dihydroisoquinolin-4-yl)ethyl)-N-methylindoline-2-carboxamide;
(3R)-N-((1R)-(6,7-difluoro-1-oxo-1,2-dihydroisoquinolin-4-yl)ethyl)-N-methyl-1,2,3,4-tetrahydroisoquinoline-3-carboxamide;
(3R)-N-((1S)-(6,7-difluoro-1-oxo-1,2-dihydroisoquinolin-4-yl)ethyl)-N-methyl-1,2,3,4-tetrahydroisoquinoline-3-carboxamide;
(3S)-N-((1R)-(6,7-difluoro-1-oxo-1,2-dihydroisoquinolin-4-yl)ethyl)-N-methyl-1,2,3,4-tetrahydroisoquinoline-3-carboxamide;
(3S)-N-((1S)-(6,7-difluoro-1-oxo-1,2-dihydroisoquinolin-4-yl)ethyl)-N-methyl-1,2,3,4-tetrahydroisoquinoline-3-carboxamide;
(R)-N-(1-(6,7-difluoro-1-oxo-1,2-dihydroisoquinolin-4-yl)ethyl)-N-methyl-4,5,6,7-tetrahydro-1H-indole-2-carboxamide;
(S)-N-(1-(6,7-difluoro-1-oxo-1,2-dihydroisoquinolin-4-yl)ethyl)-N-methyl-4,5,6,7-tetrahydro-1H-indole-2-carboxamide;
(R)-N-(1-(6,7-difluoro-1-oxo-1,2-dihydroisoquinolin-4-yl)ethyl)-N,1-dimethyl-1H-indole-2-carboxamide;
(S)-N-(1-(6,7-difluoro-1-oxo-1,2-dihydroisoquinolin-4-yl)ethyl)-N,1-dimethyl-1H-indole-2-carboxamide;
(R)-N-(1-(6,7-difluoro-1-oxo-1,2-dihydroisoquinolin-4-yl)ethyl)-N-methylimidazo[1,2-a]pyridine-2-carboxamide;
(S)-N-(1-(6,7-difluoro-1-oxo-1,2-dihydroisoquinolin-4-yl)ethyl)-N-methylimidazo[1,2-a]pyridine-2-carboxamide;
(R)-N-(1-(6,7-difluoro-1-oxo-1,2-dihydroisoquinolin-4-yl)ethyl)-N-methyl-1,4,5,6-tetrahydrocyclopenta[b]pyrrole-2-carboxamide;
(S)-N-(1-(6,7-difluoro-1-oxo-1,2-dihydroisoquinolin-4-yl)ethyl)-N-methyl-1,4,5,6-tetrahydrocyclopenta[b]pyrrole-2-carboxamide;
(R)-N-(1-(6,7-difluoro-1-oxo-1,2-dihydroisoquinolin-4-yl)ethyl)-5,6-difluoro-N-methyl-1H-benzo[d]imidazole-2-carboxamide;
(S)-N-(1-(6,7-difluoro-1-oxo-1,2-dihydroisoquinolin-4-yl)ethyl)-5,6-difluoro-N-methyl-1H-benzo[d]imidazole-2-carboxamide;
(R)-N-(1-(6,7-difluoro-1-oxo-1,2-dihydroisoquinolin-4-yl)ethyl)-N-methyl-1H-pyrrolo[2,3-b]pyridine-2-carboxamide;
(S)-N-(1-(6,7-difluoro-1-oxo-1,2-dihydroisoquinolin-4-yl)ethyl)-N-methyl-1H-pyrrolo[2,3-b]pyridine-2-carboxamide;
(R)-N-(1-(6,7-difluoro-1-oxo-1,2-dihydroisoquinolin-4-yl)ethyl)-N-methyl-1H-indole-3-carboxamide;
(S)-N-(1-(6,7-difluoro-1-oxo-1,2-dihydroisoquinolin-4-yl)ethyl)-N-methyl-1H-indole-3-carboxamide;
(R)-N-(1-(6,7-difluoro-1-oxo-1,2-dihydroisoquinolin-4-yl)ethyl)-N-methyl-1H-indazole-3-carboxamide;
(S)-N-(1-(6,7-difluoro-1-oxo-1,2-dihydroisoquinolin-4-yl)ethyl)-N-methyl-1H-indazole-3-carboxamide;
(R)-N-(1-(7,8-difluoro-1-oxo-1,2-dihydroisoquinolin-4-yl)ethyl)-N-methyl-1,4,5,6-tetrahydrocyclopenta[b]pyrrole-2-carboxamide;
(S)-N-(1-(7,8-difluoro-1-oxo-1,2-dihydroisoquinolin-4-yl)ethyl)-N-methyl-1,4,5,6-tetrahydrocyclopenta[b]pyrrole-2-carboxamide;
(R)-N-(1-(6,7-difluoro-1-oxo-1,2-dihydroisoquinolin-4-yl)ethyl)-2-(1H-indol-2-yl)-N-methylacetamide;
(S)-N-(1-(6,7-difluoro-1-oxo-1,2-dihydroisoquinolin-4-yl)ethyl)-2-(1H-indol-2-yl)-N-methylacetamide;
(R)-3-chloro-N-(1-(6,7-difluoro-1-oxo-1,2-dihydroisoquinolin-4-yl)ethyl)-N-methyl-1H-indole-2-carboxamide;
(S)-3-chloro-N-(1-(6,7-difluoro-1-oxo-1,2-dihydroisoquinolin-4-yl)ethyl)-N-methyl-1H-indole-2-carboxamide;
N-((1R)-(6,7-difluoro-1-oxo-1,2-dihydroisoquinolin-4-yl)ethyl)-5-fluoro-N-methylindoline-(2R)-carboxamide;
N-((1R)-(6,7-difluoro-1-oxo-1,2-dihydroisoquinolin-4-yl)ethyl)-5-fluoro-N-methylindoline-(2S)-carboxamide;
N-((1S)-(6,7-difluoro-1-oxo-1,2-dihydroisoquinolin-4-yl)ethyl)-5-fluoro-N-methylindoline-(2R)-carboxamide;
N-((1S)-(6,7-difluoro-1-oxo-1,2-dihydroisoquinolin-4-yl)ethyl)-5-fluoro-N-methylindoline-(2S)-carboxamide;
N-((1R)-(7,8-difluoro-1-oxo-1,2-dihydroisoquinolin-4-yl)ethyl)-4,6-difluoro-N-methylindoline-(2R)-carboxamide;
N-((1R)-(7,8-difluoro-1-oxo-1,2-dihydroisoquinolin-4-yl)ethyl)-4,6-difluoro-N-methylindoline-(2S)-carboxamide;
N-((1S)-(7,8-difluoro-1-oxo-1,2-dihydroisoquinolin-4-yl)ethyl)-4,6-difluoro-N-methylindoline-(2R)-carboxamide;
N-((1S)-(7,8-difluoro-1-oxo-1,2-dihydroisoquinolin-4-yl)ethyl)-4,6-difluoro-N-methylindoline-(2S)-carboxamide;
(R)-N-(1-(6,7-difluoro-1-oxo-1,2-dihydroisoquinolin-4-yl)ethyl)-N-methylindolizine-2-carboxamide;
(S)-N-(1-(6,7-difluoro-1-oxo-1,2-dihydroisoquinolin-4-yl)ethyl)-N-methylindolizine-2-carboxamide;
(R)-N-(1-(7,8-difluoro-1-oxo-1,2-dihydroisoquinolin-4-yl)ethyl)-N-methylindolizine-2-carboxamide;
(S)-N-(1-(7,8-difluoro-1-oxo-1,2-dihydroisoquinolin-4-yl)ethyl)-N-methylindolizine-2-carboxamide;
N-((1R)-(7,8-difluoro-1-oxo-1,2-dihydroisoquinolin-4-yl)ethyl)-5-fluoro-N-methylindoline-(2R)-carboxamide;
N-((1R)-(7,8-difluoro-1-oxo-1,2-dihydroisoquinolin-4-yl)ethyl)-5-fluoro-N-methylindoline-(2S)-carboxamide;
N-((1S)-(7,8-difluoro-1-oxo-1,2-dihydroisoquinolin-4-yl)ethyl)-5-fluoro-N-methylindoline-(2R)-carboxamide;
N-((1S)-(7,8-difluoro-1-oxo-1,2-dihydroisoquinolin-4-yl)ethyl)-5-fluoro-N-methylindoline-(2S)-carboxamide;
(R)-8-chloro-N-(1-(6,7-difluoro-1-oxo-1,2-dihydroisoquinolin-4-yl)ethyl)-N-methylindolizine-2-carboxamide;
(S)-8-chloro-N-(1-(6,7-difluoro-1-oxo-1,2-dihydroisoquinolin-4-yl)ethyl)-N-methylindolizine-2-carboxamide;
(R)-N-(1-(6,7-difluoro-1-oxo-1,2-dihydroisoquinolin-4-yl)ethyl)-8-fluoro-N-methylindolizine-2-carboxamide;
(S)-N-(1-(6,7-difluoro-1-oxo-1,2-dihydroisoquinolin-4-yl)ethyl)-8-fluoro-N-methylindolizine-2-carboxamide;
(R)-N-(1-(7,8-difluoro-1-oxo-1,2-dihydroisoquinolin-4-yl)ethyl)-8-fluoro-N-methylindolizine-2-carboxamide;
(S)-N-(1-(7,8-difluoro-1-oxo-1,2-dihydroisoquinolin-4-yl)ethyl)-8-fluoro-N-methylindolizine-2-carboxamide;
(R)-N-(1-(6,7-difluoro-1-oxo-1,2-dihydroisoquinolin-4-yl)ethyl)-N-(3-hydroxypropyl)-2-(1H-indol-2-yl)acetamide;
(S)-N-(1-(6,7-difluoro-1-oxo-1,2-dihydroisoquinolin-4-yl)ethyl)-N-(3-hydroxypropyl)-2-(1H-indol-2-yl)acetamide;
(R)-N-(1-(7,8-difluoro-1-oxo-1,2-dihydroisoquinolin-4-yl)ethyl)-N-methyl-6-oxo-1,6-dihydropyridine-2-carboxamide;
(S)-N-(1-(7,8-difluoro-1-oxo-1,2-dihydroisoquinolin-4-yl)ethyl)-N-methyl-6-oxo-1,6-dihydropyridine-2-carboxamide;
(R)-N-(1-(6,7-difluoro-1-oxo-1,2-dihydroisoquinolin-4-yl)ethyl)-5,5-difluoro-N-methyl-4,5,6,7-tetrahydro-1H-indole-2-carboxamide;
(S)-N-(1-(6,7-difluoro-1-oxo-1,2-dihydroisoquinolin-4-yl)ethyl)-5,5-difluoro-N-methyl-4,5,6,7-tetrahydro-1H-indole-2-carboxamide;
(R)-N-(1-(7,8-difluoro-1-oxo-1,2-dihydroisoquinolin-4-yl)ethyl)-5,5-difluoro-N-methyl-4,5,6,7-tetrahydro-1H-indole-2-carboxamide;
(S)-N-(1-(7,8-difluoro-1-oxo-1,2-dihydroisoquinolin-4-yl)ethyl)-5,5-difluoro-N-methyl-4,5,6,7-tetrahydro-1H-indole-2-carboxamide;
(R)-8-chloro-N-(1-(7,8-difluoro-1-oxo-1,2-dihydroisoquinolin-4-yl)ethyl)-N-methylindolizine-2-carboxamide;
(S)-8-chloro-N-(1-(7,8-difluoro-1-oxo-1,2-dihydroisoquinolin-4-yl)ethyl)-N-methylindolizine-2-carboxamide;
(R)-3-chloro-N-(1-(6,7-difluoro-1-oxo-1,2-dihydroisoquinolin-4-yl)ethyl)-N-(3-hydroxypropyl)-1H-indole-2-carboxamide;
(S)-3-chloro-N-(1-(6,7-difluoro-1-oxo-1,2-dihydroisoquinolin-4-yl)ethyl)-N-(3-hydroxypropyl)-1H-indole-2-carboxamide;
(R)-7-chloro-N-(1-(6,7-difluoro-1-oxo-1,2-dihydroisoquinolin-4-yl)ethyl)-N-methylindolizine-2-carboxamide;
(S)-7-chloro-N-(1-(6,7-difluoro-1-oxo-1,2-dihydroisoquinolin-4-yl)ethyl)-N-methylindolizine-2-carboxamide;
N-((1R)-(6,7-difluoro-1-oxo-1,2-dihydroisoquinolin-4-yl)ethyl)-4,6-difluoro-N-methylindoline-(2R)-carboxamide;
N-((1R)-(6,7-difluoro-1-oxo-1,2-dihydroisoquinolin-4-yl)ethyl)-4,6-difluoro-N-methylindoline-(2S)-carboxamide;
N-((1S)-(6,7-difluoro-1-oxo-1,2-dihydroisoquinolin-4-yl)ethyl)-4,6-difluoro-N-methylindoline-(2R)-carboxamide;
N-((1S)-(6,7-difluoro-1-oxo-1,2-dihydroisoquinolin-4-yl)ethyl)-4,6-difluoro-N-methylindoline-(2S)-carboxamide;
(R)-N-(1-(6,7-difluoro-1-oxo-1,2-dihydroisoquinolin-4-yl)ethyl)-6-fluoro-N-methylindolizine-2-carboxamide;
(S)-N-(1-(6,7-difluoro-1-oxo-1,2-dihydroisoquinolin-4-yl)ethyl)-6-fluoro-N-methylindolizine-2-carboxamide;
N-((1R)-(7,8-difluoro-1-oxo-1,2-dihydroisoquinolin-4-yl)ethyl)-N,3,3-trimethylindoline-(2R)-carboxamide;
N-((1R)-(7,8-difluoro-1-oxo-1,2-dihydroisoquinolin-4-yl)ethyl)-N,3,3-trimethylindoline-(2S)-carboxamide;
N-((1S)-(7,8-difluoro-1-oxo-1,2-dihydroisoquinolin-4-yl)ethyl)-N,3,3-trimethylindoline-(2R)-carboxamide;
N-((1S)-(7,8-difluoro-1-oxo-1,2-dihydroisoquinolin-4-yl)ethyl)-N,3,3-trimethylindoline-(2S)-carboxamide;
(R)-N-(1-(6,7-difluoro-1-oxo-1,2-dihydroisoquinolin-4-yl)ethyl)-7-fluoro-N-methylindolizine-2-carboxamide;
(S)-N-(1-(6,7-difluoro-1-oxo-1,2-dihydroisoquinolin-4-yl)ethyl)-7-fluoro-N-methylindolizine-2-carboxamide;
(R)-6-chloro-N-(1-(6,7-difluoro-1-oxo-1,2-dihydroisoquinolin-4-yl)ethyl)-N-methylindolizine-2-carboxamide;
(S)-6-chloro-N-(1-(6,7-difluoro-1-oxo-1,2-dihydroisoquinolin-4-yl)ethyl)-N-methylindolizine-2-carboxamide;
N-((1R)-(6,7-difluoro-1-oxo-1,2-dihydroisoquinolin-4-yl)ethyl)-N,3,3-trimethylindoline-(2R)-carboxamide;
N-((1R)-(6,7-difluoro-1-oxo-1,2-dihydroisoquinolin-4-yl)ethyl)-N,3,3-trimethylindoline-(2S)-carboxamide;
N-((1S)-(6,7-difluoro-1-oxo-1,2-dihydroisoquinolin-4-yl)ethyl)-N,3,3-trimethylindoline-(2R)-carboxamide;
N-((1S)-(6,7-difluoro-1-oxo-1,2-dihydroisoquinolin-4-yl)ethyl)-N,3,3-trimethylindoline-(2S)-carboxamide;
(R)-N-(1-(6,7-Difluoro-1-oxo-1,2-dihydroisoquinolin-4-yl)ethyl)-N-isobutylindolizine-2-carboxamide;
(S)-N-(1-(6,7-Difluoro-1-oxo-1,2-dihydroisoquinolin-4-yl)ethyl)-N-isobutylindolizine-2-carboxamide;
(R)-N-(1-(6,7-difluoro-1-oxo-1,2-dihydroisoquinolin-4-yl)ethyl)-3-fluoro-N-isobutyl-4-(trifluoromethyl)benzamide;
(S)-N-(1-(6,7-difluoro-1-oxo-1,2-dihydroisoquinolin-4-yl)ethyl)-3-fluoro-N-isobutyl-4-(trifluoromethyl)benzamide;
(R)-N-(1-(6,7-Difluoro-1-oxo-1,2-dihydroisoquinolin-4-yl)ethyl)-3-fluoro-N-methyl-4-(trifluoromethyl) benzamide;
(S)-N-(1-(6,7-Difluoro-1-oxo-1,2-dihydroisoquinolin-4-yl)ethyl)-3-fluoro-N-methyl-4-(trifluoromethyl) benzamide;
(R)-4-Chloro-N-(1-(6,7-difluoro-1-oxo-1,2-dihydroisoquinolin-4-yl)ethyl)-3-fluoro-N-methylbenzamide;
(S)-4-Chloro-N-(1-(6,7-difluoro-1-oxo-1,2-dihydroisoquinolin-4-yl)ethyl)-3-fluoro-N-methylbenzamide;
(R)-N-(1-(6,7-Difluoro-1-oxo-1,2-dihydroisoquinolin-4-yl)ethyl)-3,4,5-trifluoro-N-methylbenzamide;
(S)-N-(1-(6,7-Difluoro-1-oxo-1,2-dihydroisoquinolin-4-yl)ethyl)-3,4,5-trifluoro-N-methylbenzamide;
(R)-N-(1-(6,7-Difluoro-1-oxo-1,2-dihydroisoquinolin-4-yl)ethyl)-3-(difluoromethyl)-N-methylbenzamide;
(S)-N-(1-(6,7-Difluoro-1-oxo-1,2-dihydroisoquinolin-4-yl)ethyl)-3-(difluoromethyl)-N-methylbenzamide;
(R)-N-(1-(6,7-Difluoro-1-oxo-1,2-dihydroisoquinolin-4-yl)ethyl)-N-methylbenzamide;
(S)-N-(1-(6,7-Difluoro-1-oxo-1,2-dihydroisoquinolin-4-yl)ethyl)-N-methylbenzamide;
(R)-N-(1-(6,7-Difluoro-1-oxo-1,2-dihydroisoquinolin-4-yl)ethyl)-N-methyl-4-(trifluoromethyl) benzamide;
(S)-N-(1-(6,7-Difluoro-1-oxo-1,2-dihydroisoquinolin-4-yl)ethyl)-N-methyl-4-(trifluoromethyl) benzamide;
(R)-4-Chloro-N-(1-(6,7-difluoro-1-oxo-1,2-dihydroisoquinolin-4-yl)ethyl)-N-methylbenzamide;
(S)-4-Chloro-N-(1-(6,7-difluoro-1-oxo-1,2-dihydroisoquinolin-4-yl)ethyl)-N-methylbenzamide;
(R)-N-(1-(6,7-Difluoro-1-oxo-1,2-dihydroisoquinolin-4-yl)ethyl)-3-(difluoromethyl)-4-fluoro-N-methylbenzamide;
(S)-N-(1-(6,7-Difluoro-1-oxo-1,2-dihydroisoquinolin-4-yl)ethyl)-3-(difluoromethyl)-4-fluoro-N-methylbenzamide;
(R)-3-Chloro-N-(1-(6,7-difluoro-1-oxo-1,2-dihydroisoquinolin-4-yl)ethyl)-4-fluoro-N-methylbenzamide;
(S)-3-Chloro-N-(1-(6,7-difluoro-1-oxo-1,2-dihydroisoquinolin-4-yl)ethyl)-4-fluoro-N-methylbenzamide;
(R)-N-(1-(6,7-Difluoro-1-oxo-1,2-dihydroisoquinolin-4-yl)ethyl)-3,4-difluoro-N-methylbenzamide;
(S)-N-(1-(6,7-Difluoro-1-oxo-1,2-dihydroisoquinolin-4-yl)ethyl)-3,4-difluoro-N-methylbenzamide;
(R)-N-(1-(6,7-Difluoro-1-oxo-1,2-dihydroisoquinolin-4-yl)ethyl)-N,1-dimethyl-1H-indole-5-carboxamide;
(S)-N-(1-(6,7-Difluoro-1-oxo-1,2-dihydroisoquinolin-4-yl)ethyl)-N,1-dimethyl-1H-indole-5-carboxamide;
(R)-N-(1-(6,7-Difluoro-1-oxo-1,2-dihydroisoquinolin-4-yl)ethyl)-N,1-dimethyl-1H-indole-6-carboxamide;
(S)-N-(1-(6,7-Difluoro-1-oxo-1,2-dihydroisoquinolin-4-yl)ethyl)-N,1-dimethyl-1H-indole-6-carboxamide;
(R)-N1-Cyclopropyl-N2-(1-(6,7-difluoro-1-oxo-1,2-dihydroisoquinolin-4-yl)ethyl)-N2-methyloxalamide;
(S)-N1-Cyclopropyl-N2-(1-(6,7-difluoro-1-oxo-1,2-dihydroisoquinolin-4-yl)ethyl)-N2-methyloxalamide;
(R)-N1-(3-Chloro-4-fluorophenyl)-N2-(1-(6,7-difluoro-1-oxo-1,2-dihydroisoquinolin-4-yl)ethyl)-N2-methyloxalamide;
(S)-N1-(3-Chloro-4-fluorophenyl)-N2-(1-(6,7-difluoro-1-oxo-1,2-dihydroisoquinolin-4-yl)ethyl)-N2-methyloxalamide;
(R)-N-(1-(7,8-difluoro-1-oxo-1,2-dihydroisoquinolin-4-yl)ethyl)-3-fluoro-N-isobutyl-4-(trifluoromethyl)benzamide;
(S)-N-(1-(7,8-difluoro-1-oxo-1,2-dihydroisoquinolin-4-yl)ethyl)-3-fluoro-N-isobutyl-4-(trifluoromethyl)benzamide;
(R)-3-fluoro-N-methyl-N-(1-(1-oxo-1,2-dihydroisoquinolin-4-yl)ethyl)-4-(trifluoromethyl)benzamide;
(S)-3-fluoro-N-methyl-N-(1-(1-oxo-1,2-dihydroisoquinolin-4-yl)ethyl)-4-(trifluoromethyl)benzamide;
(R)-4-bromo-N-methyl-N-(1-(1-oxo-1,2-dihydroisoquinolin-4-yl)ethyl)benzamide;
(S)-4-bromo-N-methyl-N-(1-(1-oxo-1,2-dihydroisoquinolin-4-yl)ethyl)benzamide;
(R)-N-methyl-N-(1-(1-oxo-1,2-dihydroisoquinolin-4-yl)ethyl)-4-(trifluoromethyl)benzamide;
(S)-N-methyl-N-(1-(1-oxo-1,2-dihydroisoquinolin-4-yl)ethyl)-4-(trifluoromethyl)benzamide;
(R)-4-chloro-3-fluoro-N-methyl-N-(1-(1-oxo-1,2-dihydroisoquinolin-4-yl)ethyl)benzamide;
(S)-4-chloro-3-fluoro-N-methyl-N-(1-(1-oxo-1,2-dihydroisoquinolin-4-yl)ethyl)benzamide;
(R)-3-chloro-4-fluoro-N-methyl-N-(1-(1-oxo-1,2-dihydroisoquinolin-4-yl)ethyl)benzamide;
(S)-3-chloro-4-fluoro-N-methyl-N-(1-(1-oxo-1,2-dihydroisoquinolin-4-yl)ethyl)benzamide;
(R)-4-bromo-3-fluoro-N-methyl-N-(1-(1-oxo-1,2-dihydroisoquinolin-4-yl)ethyl)benzamide;
(S)-4-bromo-3-fluoro-N-methyl-N-(1-(1-oxo-1,2-dihydroisoquinolin-4-yl)ethyl)benzamide;
(R)-2-(4-chlorophenyl)-N-methyl-N-(1-(1-oxo-1,2-dihydroisoquinolin-4-yl)ethyl)acetamide;
(S)-2-(4-chlorophenyl)-N-methyl-N-(1-(1-oxo-1,2-dihydroisoquinolin-4-yl)ethyl)acetamide;
(R)-N-methyl-N-(1-(1-oxo-1,2-dihydroisoquinolin-4-yl)ethyl)indolizine-2-carboxamide;
(S)-N-methyl-N-(1-(1-oxo-1,2-dihydroisoquinolin-4-yl)ethyl)indolizine-2-carboxamide;
(R)-7-fluoro-N-methyl-N-(1-(1-oxo-1,2-dihydroisoquinolin-4-yl)ethyl)indolizine-2-carboxamide;
(S)-7-fluoro-N-methyl-N-(1-(1-oxo-1,2-dihydroisoquinolin-4-yl)ethyl)indolizine-2-carboxamide;
(R)-8-chloro-N-methyl-N-(1-(1-oxo-1,2-dihydroisoquinolin-4-yl)ethyl)indolizine-2-carboxamide;
(S)-8-chloro-N-methyl-N-(1-(1-oxo-1,2-dihydroisoquinolin-4-yl)ethyl)indolizine-2-carboxamide;
(R)-N-methyl-N-(1-(1-oxo-1,2-dihydroisoquinolin-4-yl)ethyl)-1H-indole-2-carboxamide;
(S)-N-methyl-N-(1-(1-oxo-1,2-dihydroisoquinolin-4-yl)ethyl)-1H-indole-2-carboxamide;
(R)-4-fluoro-N-methyl-N-(1-(1-oxo-1,2-dihydroisoquinolin-4-yl)ethyl)-1H-indole-2-carboxamide;
(S)-4-fluoro-N-methyl-N-(1-(1-oxo-1,2-dihydroisoquinolin-4-yl)ethyl)-1H-indole-2-carboxamide;
(R)-5-fluoro-N-methyl-N-(1-(1-oxo-1,2-dihydroisoquinolin-4-yl)ethyl)-1H-indole-2-carboxamide;
(S)-5-fluoro-N-methyl-N-(1-(1-oxo-1,2-dihydroisoquinolin-4-yl)ethyl)-1H-indole-2-carboxamide;
(R)-4,6-difluoro-N-methyl-N-(1-(1-oxo-1,2-dihydroisoquinolin-4-yl)ethyl)-1H-indole-2-carboxamide;
(S)-4,6-difluoro-N-methyl-N-(1-(1-oxo-1,2-dihydroisoquinolin-4-yl)ethyl)-1H-indole-2-carboxamide;
(R)-5,6-difluoro-N-methyl-N-(1-(1-oxo-1,2-dihydroisoquinolin-4-yl)ethyl)-1H-indole-2-carboxamide;
(S)-5,6-difluoro-N-methyl-N-(1-(1-oxo-1,2-dihydroisoquinolin-4-yl)ethyl)-1H-indole-2-carboxamide;
(R)-N-methyl-N-((R)-1-(1-oxo-1,2-dihydroisoquinolin-4-yl)ethyl)indoline-2-carboxamide;
(S)-N-methyl-N-((R)-1-(1-oxo-1,2-dihydroisoquinolin-4-yl)ethyl)indoline-2-carboxamide;
(R)-4-bromo-N-(1-(6,7-difluoro-2-methyl-1-oxo-1,2-dihydroisoquinolin-4-yl)ethyl)-3-fluoro-N-methylbenzamide;
(S)-4-bromo-N-(1-(6,7-difluoro-2-methyl-1-oxo-1,2-dihydroisoquinolin-4-yl)ethyl)-3-fluoro-N-methylbenzamide;
(R)-4-bromo-N-(1-(6,7-difluoro-1-methoxyisoquinolin-4-yl)ethyl)-N-methylbenzamide;
(S)-4-bromo-N-(1-(6,7-difluoro-1-methoxyisoquinolin-4-yl)ethyl)-N-methylbenzamide;
(R)-N-(1-(6,7-difluoro-2-methyl-1-oxo-1,2-dihydroisoquinolin-4-yl)ethyl)-3-fluoro-N-methyl-4-(trifluoromethyl)benzamide;
(S)-N-(1-(6,7-difluoro-2-methyl-1-oxo-1,2-dihydroisoquinolin-4-yl)ethyl)-3-fluoro-N-methyl-4-(trifluoromethyl)benzamide;
(R)-4-bromo-N-(1-(6,7-difluoro-2-methyl-1-oxo-1,2-dihydroisoquinolin-4-yl)ethyl)-N-methylbenzamide;
(S)-4-bromo-N-(1-(6,7-difluoro-2-methyl-1-oxo-1,2-dihydroisoquinolin-4-yl)ethyl)-N-methylbenzamide;
(R)-N-(1-(6,7-difluoro-1-methoxyisoquinolin-4-yl)ethyl)-5,6-difluoro-N-methyl-1H-indole-2-carboxamide;
(S)-N-(1-(6,7-difluoro-1-methoxyisoquinolin-4-yl)ethyl)-5,6-difluoro-N-methyl-1H-indole-2-carboxamide;
(R)-N-(1-(6,7-difluoro-2-methyl-1-oxo-1,2-dihydroisoquinolin-4-yl)ethyl)-8-fluoro-N-methylindolizine-2-carboxamide;
(S)-N-(1-(6,7-difluoro-2-methyl-1-oxo-1,2-dihydroisoquinolin-4-yl)ethyl)-8-fluoro-N-methylindolizine-2-carboxamide;
(R)-N-(1-(6,7-difluoro-2-methyl-1-oxo-1,2-dihydroisoquinolin-4-yl)ethyl)-5,6-difluoro-N-methyl-1H-indole-2-carboxamide;
(S)-N-(1-(6,7-difluoro-2-methyl-1-oxo-1,2-dihydroisoquinolin-4-yl)ethyl)-5,6-difluoro-N-methyl-1H-indole-2-carboxamide;
(R)-N-(1-(6,7-difluoro-1-methoxyisoquinolin-4-yl)ethyl)-3-fluoro-N-methyl-4-(trifluoromethyl)benzamide;
(S)-N-(1-(6,7-difluoro-1-methoxyisoquinolin-4-yl)ethyl)-3-fluoro-N-methyl-4-(trifluoromethyl)benzamide;
(R)-4-bromo-N-(1-(6,7-difluoro-1-methoxyisoquinolin-4-yl)ethyl)-3-fluoro-N-methylbenzamide;
(S)-4-bromo-N-(1-(6,7-difluoro-1-methoxyisoquinolin-4-yl)ethyl)-3-fluoro-N-methylbenzamide;
(R)-N-(1-(6,7-difluoro-1-methoxyisoquinolin-4-yl)ethyl)-8-fluoro-N-methylindolizine-2-carboxamide;
(S)-N-(1-(6,7-difluoro-1-methoxyisoquinolin-4-yl)ethyl)-8-fluoro-N-methylindolizine-2-carboxamide;
(2S)-N-(1-(6,7-difluoro-1-methoxyisoquinolin-4-yl)ethyl)-N-methylindoline-2-carboxamide;
(S)-N-((S)-1-(6,7-difluoro-1-methoxyisoquinolin-4-yl)ethyl)-N-methylindoline-2-carboxamide;
(S)-N-((R)-1-(6,7-difluoro-1-methoxyisoquinolin-4-yl)ethyl)-N-methylindoline-2-carboxamide;
(2R)-N-(1-(6,7-difluoro-1-methoxyisoquinolin-4-yl)ethyl)-N-methylindoline-2-carboxamide;
(R)-N-((S)-1-(6,7-difluoro-1-methoxyisoquinolin-4-yl)ethyl)-N-methylindoline-2-carboxamide;
(R)-N-((R)-1-(6,7-difluoro-1-methoxyisoquinolin-4-yl)ethyl)-N-methylindoline-2-carboxamide;
(2S)-N-(1-(6,7-difluoro-2-methyl-1-oxo-1,2-dihydroisoquinolin-4-yl)ethyl)-N-methylindoline-2-carboxamide;
(S)-N-((S)-1-(6,7-difluoro-2-methyl-1-oxo-1,2-dihydroisoquinolin-4-yl)ethyl)-N-methylindoline-2-carboxamide;
(S)-N-((R)-1-(6,7-difluoro-2-methyl-1-oxo-1,2-dihydroisoquinolin-4-yl)ethyl)-N-methylindoline-2-carboxamide;
(2R)-N-(1-(6,7-difluoro-2-methyl-1-oxo-1,2-dihydroisoquinolin-4-yl)ethyl)-N-methylindoline-2-carboxamide;
(R)-N-((S)-1-(6,7-difluoro-2-methyl-1-oxo-1,2-dihydroisoquinolin-4-yl)ethyl)-N-methylindoline-2-carboxamide;
(R)-N-((R)-1-(6,7-difluoro-2-methyl-1-oxo-1,2-dihydroisoquinolin-4-yl)ethyl)-N-methylindoline-2-carboxamide;
(R)-N-(1-(6,7-difluoro-1-oxo-1,2-dihydroisoquinolin-4-yl)ethyl)-N-methylindolizine-6-carboxamide;
(S)-N-(1-(6,7-difluoro-1-oxo-1,2-dihydroisoquinolin-4-yl)ethyl)-N-methylindolizine-6-carboxamide;
(R)-2-chloro-N-(1-(6,7-difluoro-1-oxo-1,2-dihydroisoquinolin-4-yl)ethyl)-N-methyl-4H-thieno[3,2-b]pyrrole-5-carboxamide;
(S)-2-chloro-N-(1-(6,7-difluoro-1-oxo-1,2-dihydroisoquinolin-4-yl)ethyl)-N-methyl-4H-thieno[3,2-b]pyrrole-5-carboxamide;
(R)-N-(1-(6,7-difluoro-1-oxo-1,2-dihydroisoquinolin-4-yl)ethyl)-N-methylindolizine-7-carboxamide; and
(S)-N-(1-(6,7-difluoro-1-oxo-1,2-dihydroisoquinolin-4-yl)ethyl)-N-methylindolizine-7-carboxamide;
or a salt, solvate, prodrug, isotopically labeled derivative, stereoisomer, or tautomer thereof, or any mixtures thereof.

17. A pharmaceutical composition comprising at least one compound of claim 1 and a pharmaceutically acceptable carrier.

18. The pharmaceutical composition of claim 17, further comprising at least one additional agent useful for treating, ameliorating, or preventing hepatitis infection.

19. The pharmaceutical composition of claim 18, wherein the at least one additional agent comprises at least one selected from the group consisting of reverse transcriptase inhibitor; capsid inhibitor; cccDNA formation inhibitor; RNA destabilizer; oligomeric nucleotide targeted against the HBV genome; immunostimulator; GalNAc-siRNA conjugate targeted against an HBV gene transcript; and therapeutic vaccine.

20. The pharmaceutical composition of claim 19, wherein the immunostimulator is a checkpoint inhibitor, optionally wherein the checkpoint inhibitor is a PD-L1 inhibitor.

21. (canceled)

22. A method of treating, ameliorating, or preventing hepatitis B virus (HBV) infection in a subject, the method comprising administering to the subject in need thereof a therapeutically effective amount of at least one compound of claim 1, optionally wherein the composition is administered to the subject in a pharmaceutically acceptable composition.

23. The method of claim 22, wherein the subject is further infected with hepatitis D virus (HDV).

24. (canceled)

25. The method of claim 22, wherein the subject is further administered at least one additional agent useful for treating, ameliorating, or preventing the hepatitis B virus infection.

26. The method of claim 25, wherein the at least one additional agent comprises at least one selected from the group consisting of reverse transcriptase inhibitor; capsid inhibitor; cccDNA formation inhibitor; RNA destabilizer; oligomeric nucleotide targeted against the HBV genome; immunostimulator; GalNAc-siRNA conjugate targeted against an HBV gene transcript; and therapeutic vaccine.

27. The method of claim 26, wherein the immunostimulator is a checkpoint inhibitor, optionally wherein the checkpoint inhibitor is a PD-L1 inhibitor.

28. (canceled)

29. The method of claim 25, wherein the subject is co-administered the at least one compound and the at least one additional agent, optionally wherein the at least one compound and the at least one additional agent are coformulated.

30. (canceled)

31. A method of inhibiting expression or function of a viral capsid protein directly or indirectly in a hepatitis B virus-infected subject, the method comprising administering to the subject in need thereof a therapeutically effective amount of at least one compound of claim 1, optionally wherein the composition is administered to the subject in a pharmaceutically acceptable composition.

32. The method of claim 31, wherein the subject is further infected with hepatitis D virus (HDV).

33. (canceled)

34. The method of claim 31, wherein the subject is further administered at least one additional agent useful for treating, ameliorating, or preventing the hepatitis B viral infection.

35. The method of claim 34, wherein the at least one additional agent comprises at least one selected from the group consisting of reverse transcriptase inhibitor; capsid inhibitor; cccDNA formation inhibitor; RNA destabilizer; oligomeric nucleotide targeted against the HBV genome; immunostimulator; GalNAc-siRNA conjugate targeted against an HBV gene transcript; and therapeutic vaccine.

36. The method of claim 35, wherein the immunostimulator is a checkpoint inhibitor, optionally wherein the checkpoint inhibitor is a PD-L1 inhibitor.

37. (canceled)

38. The method of claim 34, wherein the subject is co-administered the at least one compound and the at least one additional agent, optionally wherein the at least one compound and the at least one additional agent are coformulated.

39. (canceled)

40. The method of claim 22, wherein the subject is a mammal, optionally wherein the mammal is a human.

41. (canceled)
